(19) United States
(12) Patent Application Publication
(10) Pub. No.: US 2023/0255110 A1
(43) Pub. Date: Aug. 10, 2023

(54) ORGANIC ELECTROLUMINESCENT COMPOUND AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: So-Young JUNG, Gyeonggi-do (KR); Su-Hyun LEE, Gyeonggi-do (KR); Sung-Wook CHO, Gyeonggi-do (KR)

(21) Appl. No.: 18/192,841

(22) Filed: Mar. 30, 2023

Related U.S. Application Data
(63) Continuation of application No. 16/640,078, filed on Feb. 19, 2020, now abandoned.

(30) Foreign Application Priority Data
Sep. 11, 2017 (KR) 10-2017-0116154
Jul. 12, 2018 (KR) 10-2018-0081133

(51) Int. Cl. H10K 85/60; C09K 11/06

(52) U.S. Cl. CPC H10K 85/6572; C09K 11/06; H10K 85/6574; H10K 85/6576; C09K 2211/1059; C09K 2211/1074

(57) ABSTRACT

The present disclosure relates to an organic electroluminescent compound, and an organic electroluminescent device comprising the same. By comprising the organic electroluminescent compound of the present disclosure, an organic electroluminescent device having improved driving voltage, luminous efficiency, lifespan characteristic, and/or power efficiency can be provided.

### CLAIM OF BENEFIT OF PRIOR APPLICATION

This application claims priority under 35 U.S.C. § 120 from U.S. patent application Ser. No. 16/640,078, filed Feb. 19, 2020, which is the National Stage Entry of PCT/KR2018/009029, filed Aug. 8, 2018, all of which are incorporated by reference herein in their entirety.

### TECHNICAL FIELD

The present disclosure relates to an organic electroluminescent compound and an organic electroluminescent device comprising the same.

### BACKGROUND ART

An electroluminescent device (EL device) is a self-light-emitting device which has advantages in that it provides a wider viewing angle, a greater contrast ratio, and a faster response time. The first organic EL device was developed by Eastman Kodak in 1987, by using small aromatic diamine molecules and aluminum complexes as materials for forming a light-emitting layer [Appl. Phys. Lett. 51, 913, 1987].

The most important factor in determining luminous efficiency in an organic electroluminescent device is light-emitting materials. Until now, fluorescent materials have been widely used as a light-emitting material. However, in view of electroluminescent mechanisms, since phosphorescent light-emitting materials theoretically enhance luminous efficiency by four (4) times compared to fluorescent light-emitting materials, development of phosphorescent light-emitting materials are widely being researched. To date, iridium(III) complexes have been widely known as phosphorescent light-emitting materials, including bis(2-(2′-benzothienyl)-pyridinato-N,C3′)iridium(acetylacetonate) ((acac)Ir(btp)₂), tris(2-phenylpyridine)iridium (Ir(ppy)₃) and bis(4,6-difluorophenylpyridinato-N,C2)picolinate iridium (Firpic) as red, green, and blue materials, respectively.

At present, 4,4′-N,N′-dicarbazol-biphenyl (CBP) is the most widely known phosphorescent host material. Recently, Pioneer (Japan), et al. developed a high performance organic electroluminescent device using bathocuproine (BCP) and aluminum(III)bis(2-methyl-8-quinolinate)(4-phenylphenolate) (BAlq) etc., which were used as hole blocking layer materials, as host materials.

Although these materials provide good light-emitting characteristics, they have the following disadvantages: (1) Due to their low glass transition temperature and poor thermal stability, their degradation may occur during a high-temperature deposition process in a vacuum, and the lifespan of the device decreases. (2) The power efficiency of an organic electroluminescent device is given by [(π/voltage)×current efficiency], and the power efficiency is inversely proportional to the voltage. Although an organic electroluminescent device comprising phosphorescent host materials provides higher current efficiency (cd/A) than one comprising fluorescent materials, a significantly high driving voltage is necessary. Thus, there is no merit in terms of power efficiency (Im/W). (3) Further, when these materials are used in an organic electroluminescent device, the operational lifespan of an organic electroluminescent device is short and luminous efficiency is still required to be improved.

Many materials or concepts for the organic layer of the organic electroluminescent device have been suggested in order to improve luminous efficiency, driving voltage, and/or lifespan. However, they were not satisfactory to put into practical use.

Korean Patent Appln. Laying-Open No. KR 2017-0035232 A discloses a compound comprising a carbazole and a nitrogen-containing heteroaryl as a light-emitting material of an organic electroluminescent device. However, the compound disclosed in said reference has a different structure from the compound of the present disclosure. Further, it is not sufficiently satisfactory in terms of lifespan characteristics of the device.

### DISCLOSURE OF THE INVENTION

### Problems to be Solved

The objective of the present disclosure is to i) provide an organic electroluminescent compound which can efficiently produce an organic electroluminescent device having improved driving voltage, luminous efficiency, lifespan characteristic, and/or power efficiency and ii) provide an organic electroluminescent device comprising the organic electroluminescent compound.

### Solution to Problems

The present inventors found that when using a carbazole derivative comprising a certain quinoxaline, quinazoline, etc., in an organic electroluminescent device, an excellent lifespan characteristic is obtained but exhibits high driving voltage, and thus by fusing a ring to the carbazole structure, the driving voltage can be lowered, and the power efficiency can be increased as well.

More specifically, the present inventors found that the above objective can be achieved by an organic electroluminescent compound represented by the following formula 1:

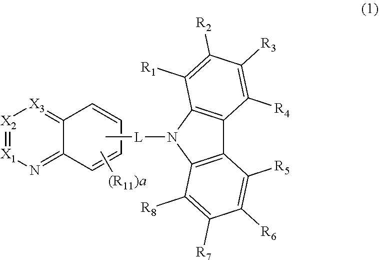

wherein
X₁ to X₃ each independently represent N or CR;
R and R₁₁ each independently represent hydrogen, deuterium, a halogen, a cyano, a substituted or unsubstituted (C1-C30)alkyl, a substituted or unsubstituted (C6-C30)aryl, or a substituted or unsubstituted (3- to 30-membered)heteroaryl;
L represents a single bond, a substituted or unsubstituted (C6-C30)arylene, or a substituted or unsubstituted (3- to 30-membered)heteroarylene; R₁ to R₈ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, or a substituted or unsubstituted (C1-C30)alkyl(C6-C30)arylamino; or may be linked to an adjacent substituent to form a ring;
at least one pair of R₁ to R₈ must be linked to each other to form a ring; and
a represents an integer of 1 to 3, where a is an integer of 2 or more, each R₁₁ may be the same or different.

### Effects of the Invention

By using the organic electroluminescent compound according to the present disclosure, an organic electroluminescent device having similar or lower driving voltage, high luminous efficiency, excellent lifespan characteristic, and/or high power efficiency can be produced.

### EMBODIMENTS OF THE INVENTION

Hereinafter, the present disclosure will be described in detail. However, the following description is intended to explain the disclosure, and it is not meant in any way to restrict the scope of the disclosure.

The term “organic electroluminescent compound” in the present disclosure means a compound that may be used in an organic electroluminescent device, and may be comprised in any layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material, an electron buffer material, a hole blocking material, an electron transport material, or an electron injection material.

The organic electroluminescent material of the present disclosure may comprise at least one compound represented by formula 1. The compound of formula 1 may be comprised in a light-emitting layer, but is not limited thereto. When comprised in the light-emitting layer, the compound of formula 1 can be comprised as a host.

Hereinafter, the compound represented by formula 1 will be described in detail.

In formula 1,

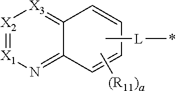

may be represented by any one of the following formulas:

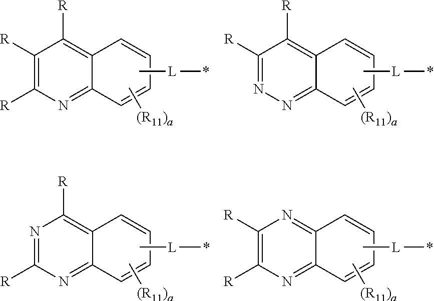

wherein
R, L, R₁₁, and a are as defined in formula 1.
* represents a connection with an adjacent substituent in a simple manner, and is the same hereinafter.

In addition, in formula 1,

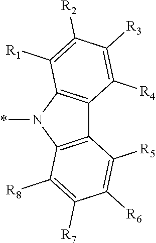

may be represented by any one of the following formulas:

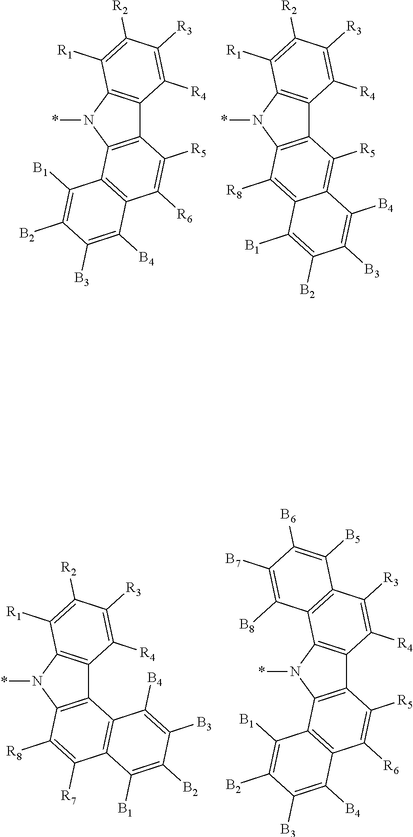

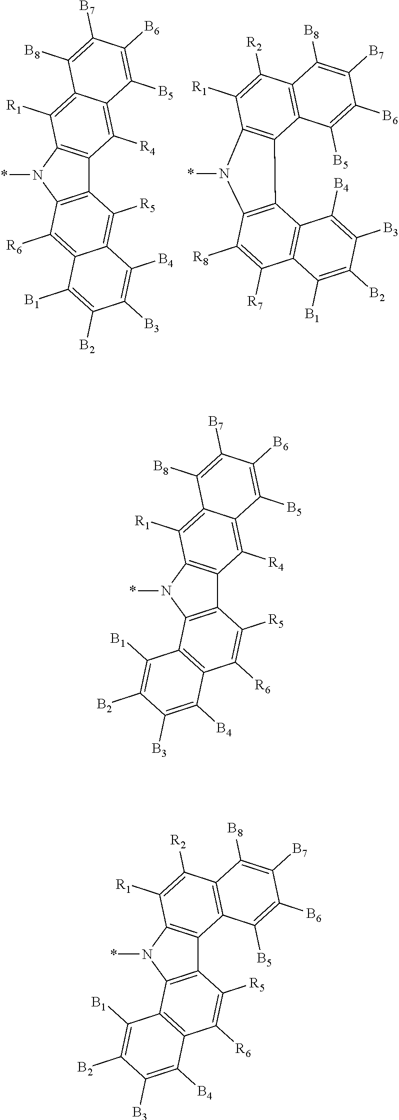

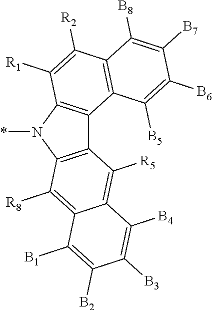

wherein
B₁ to B₈ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, or a substituted or unsubstituted (C1-C30)alkyl(C6-C30)arylamino; and
R₁ to R₈ are as defined in formula 1.

In addition, in formula 1,

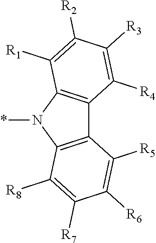

may be represented by any one of the following formulas:

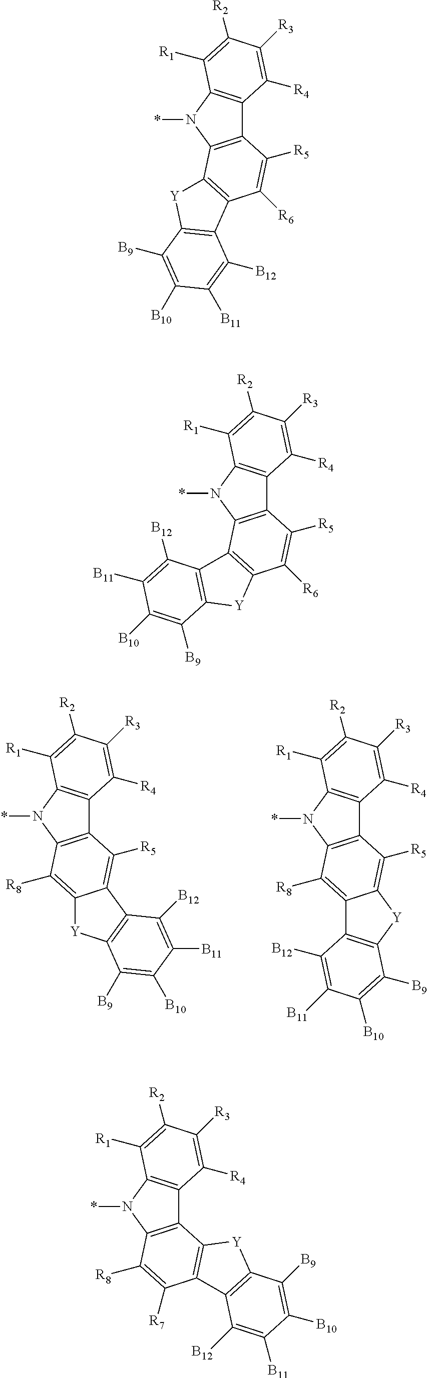

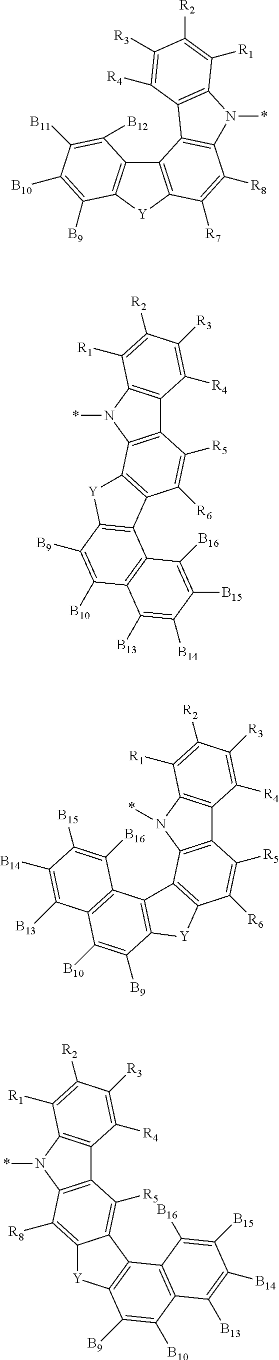

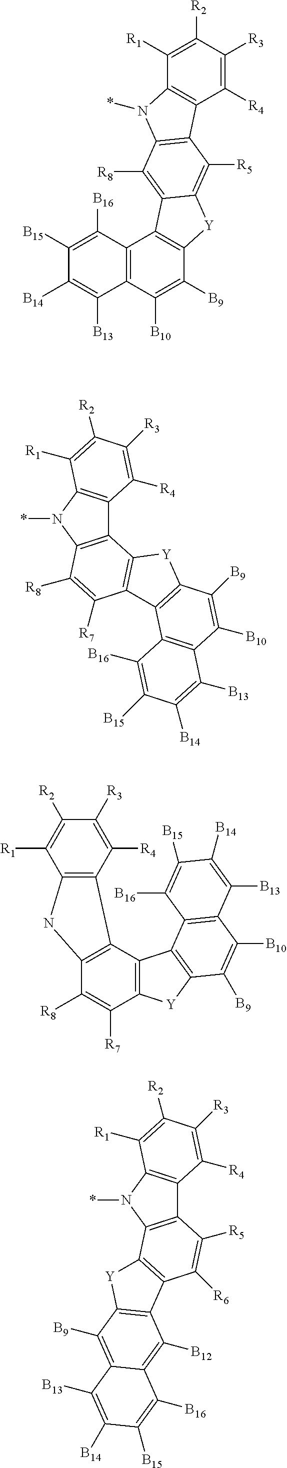

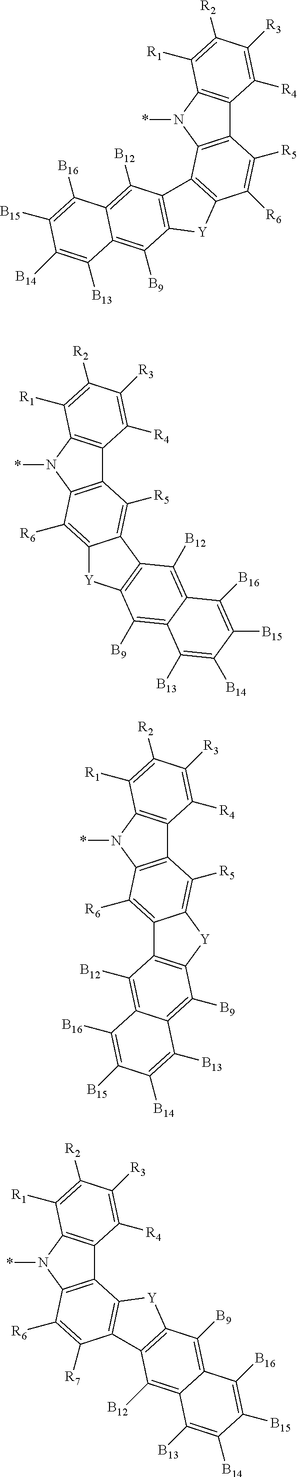

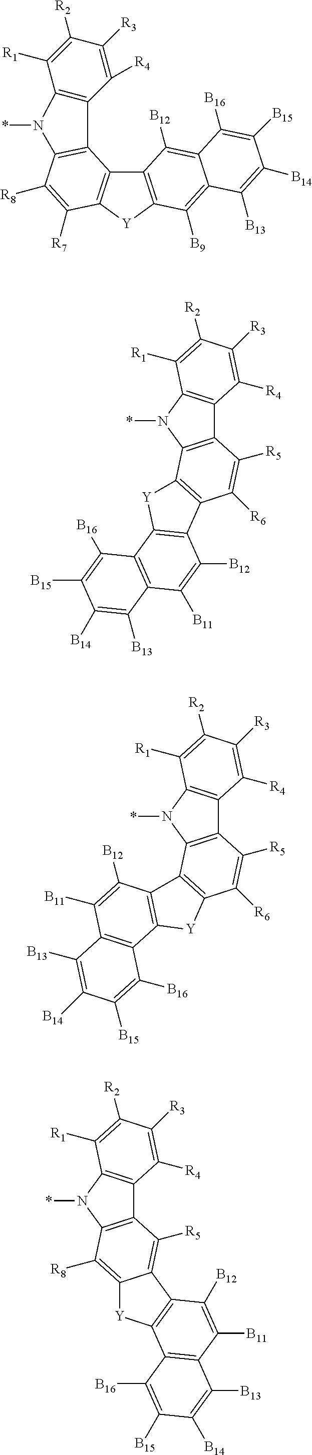

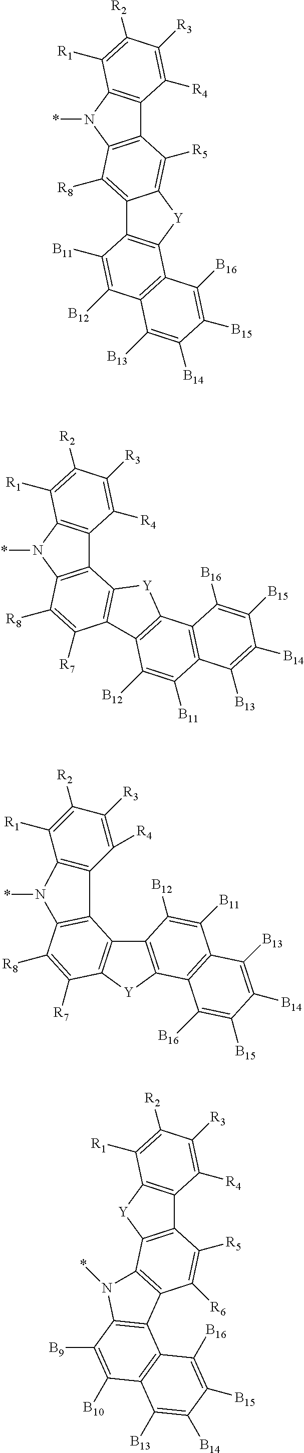

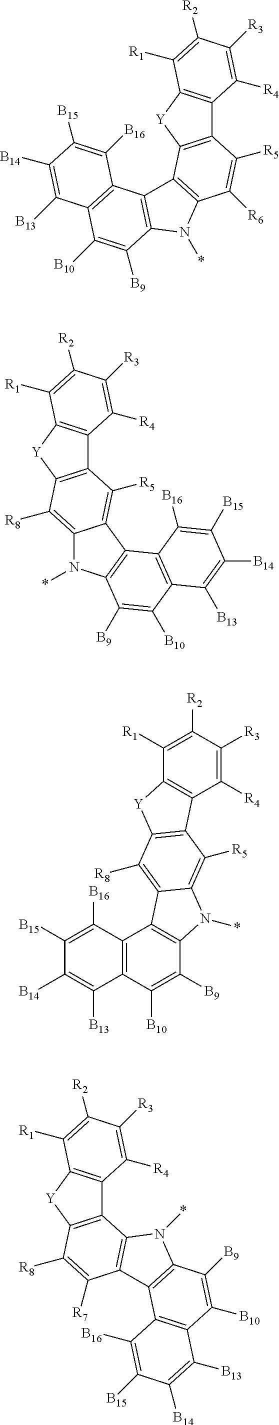

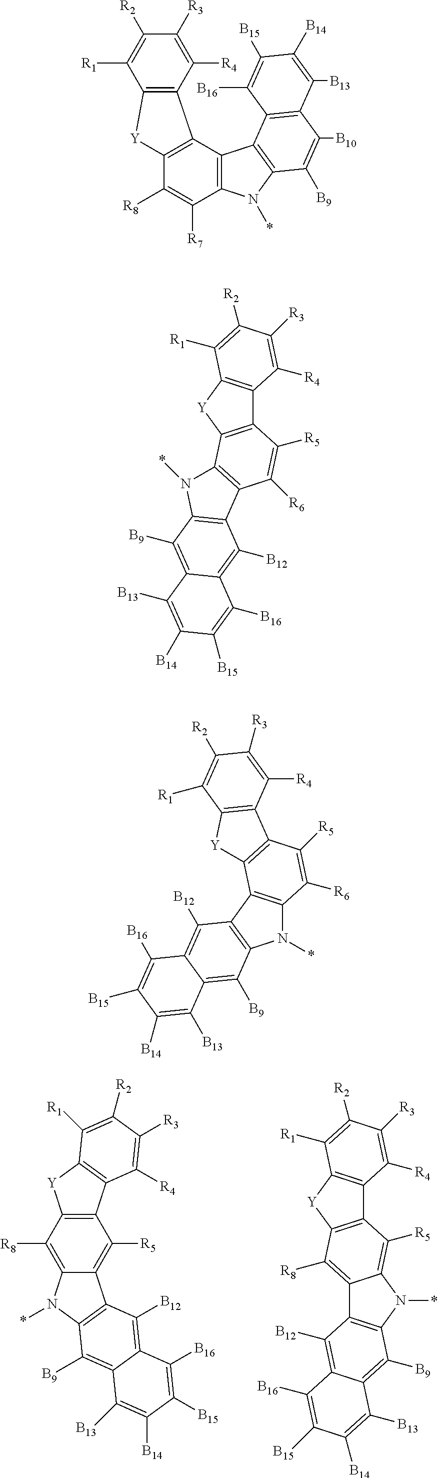

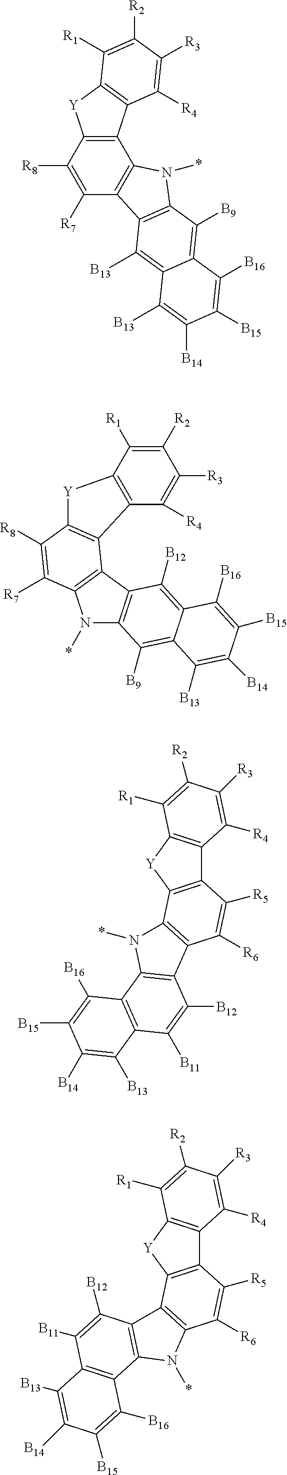

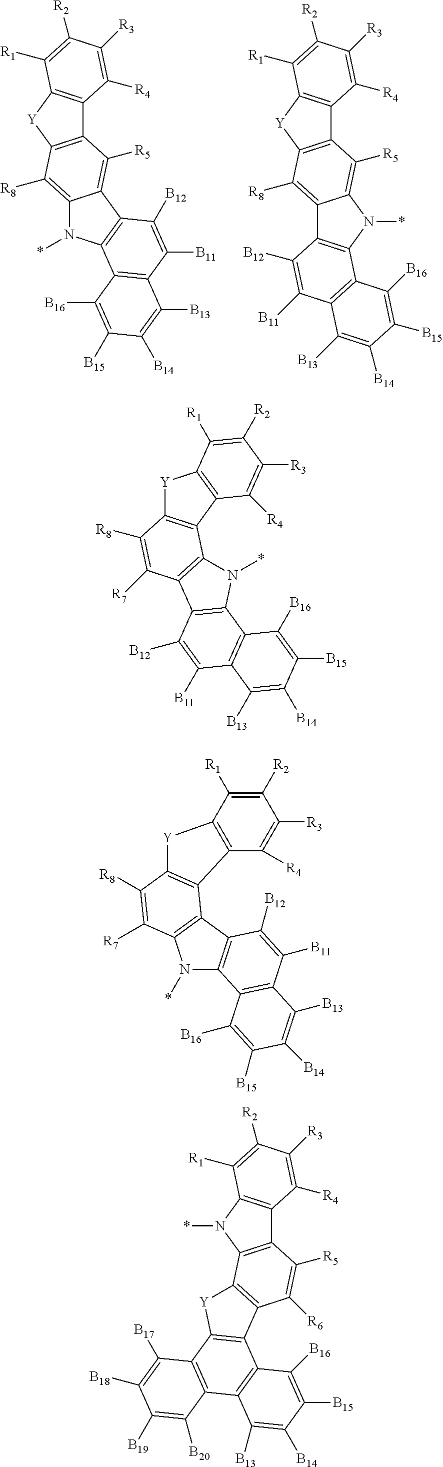

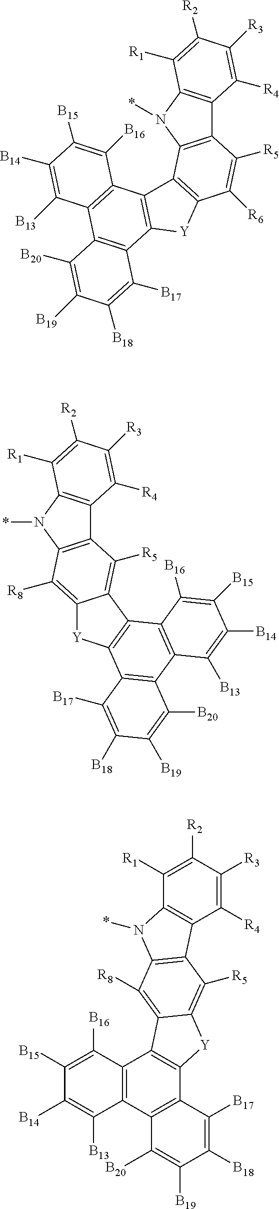

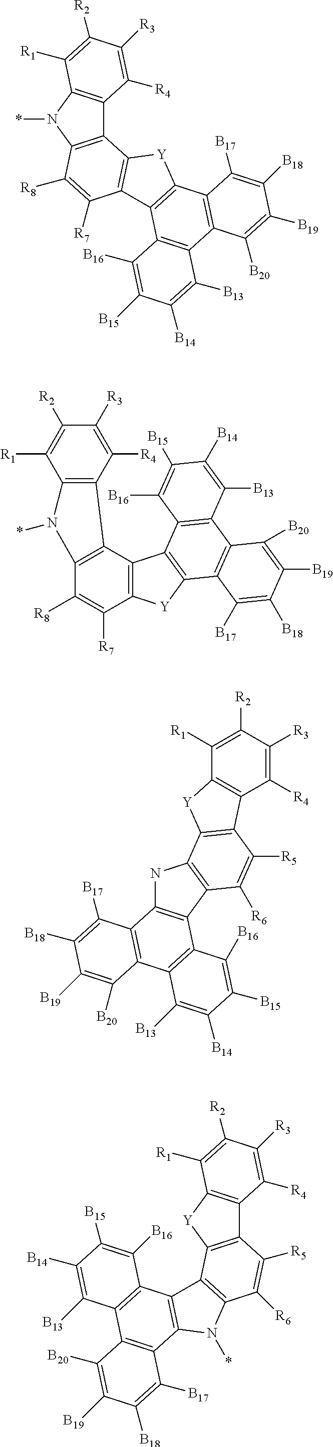

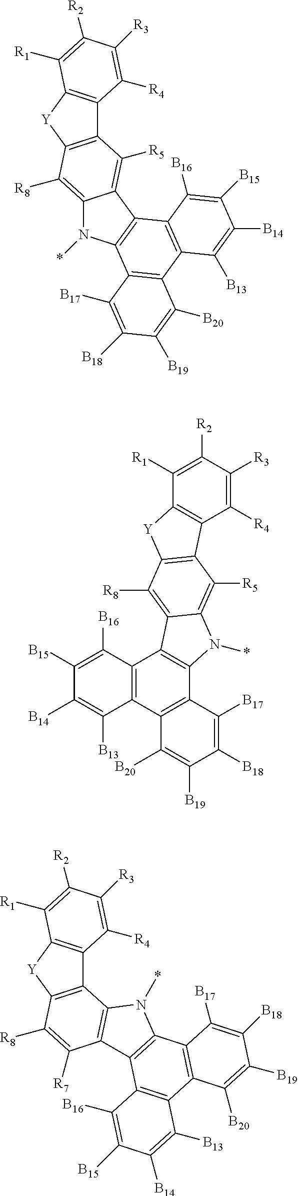

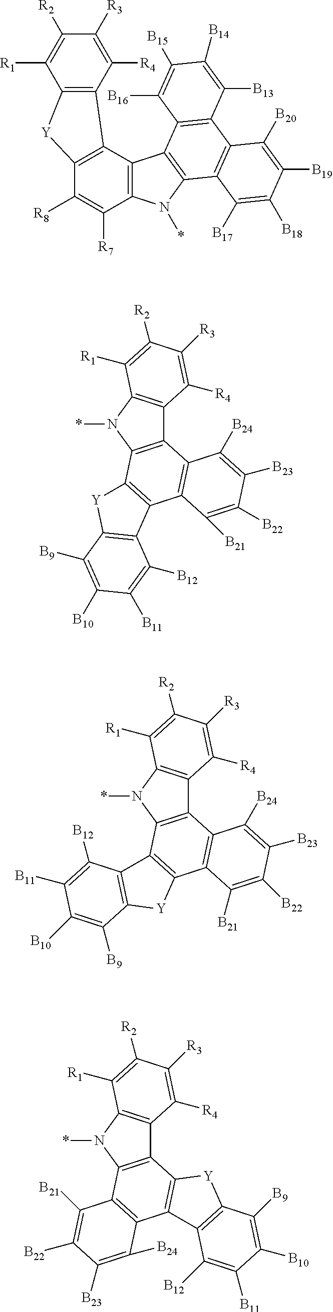

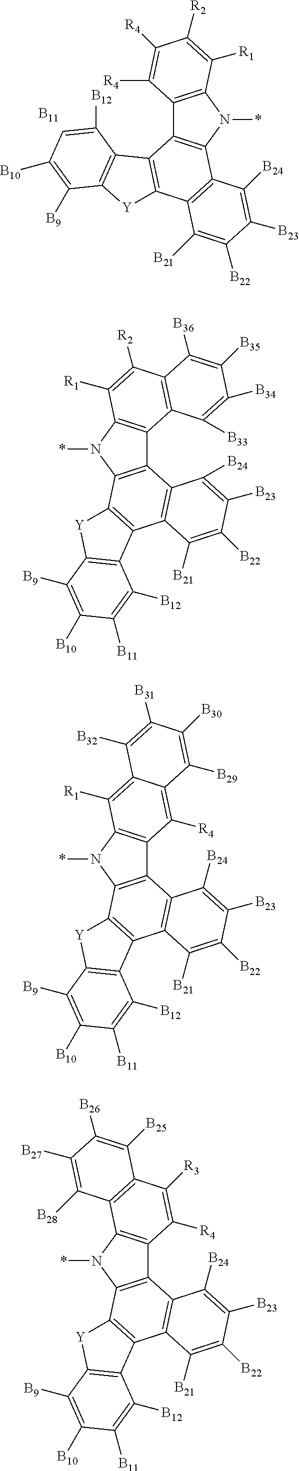

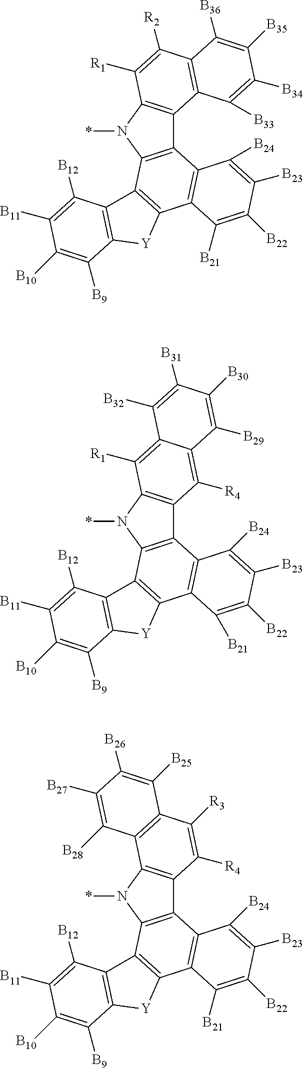

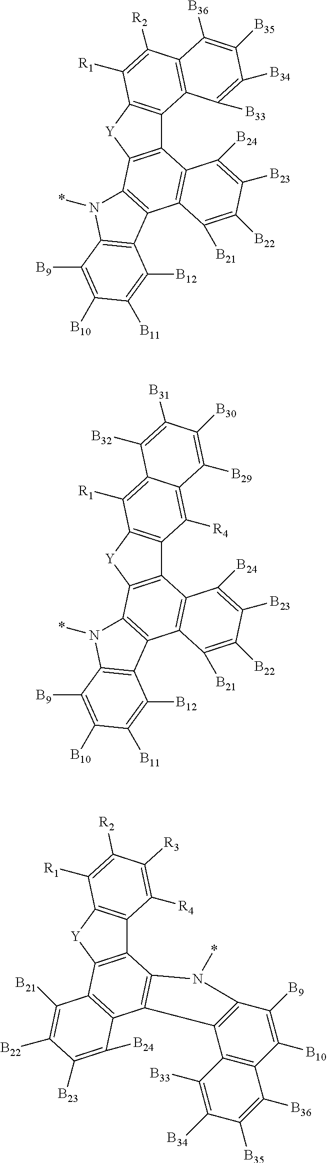

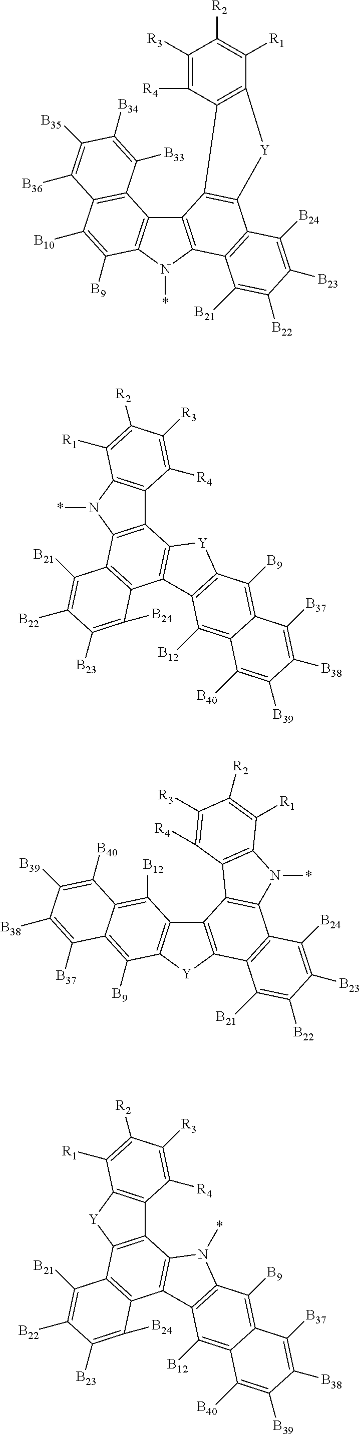

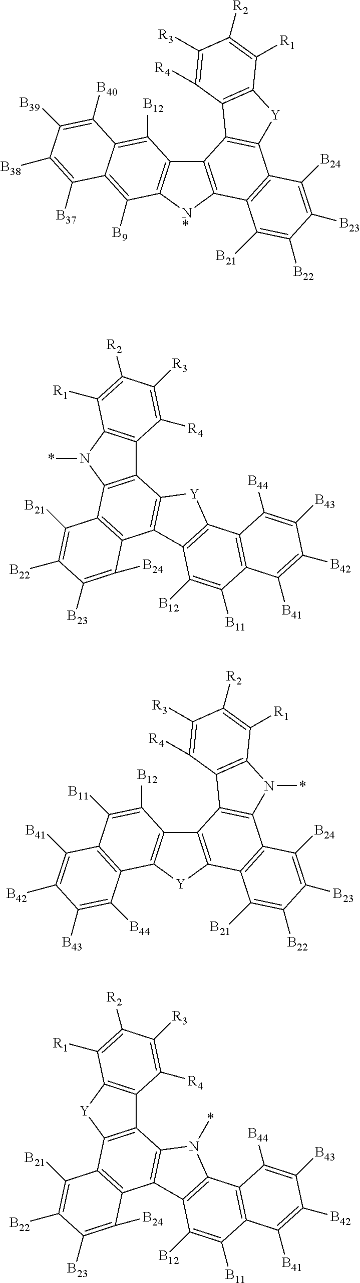

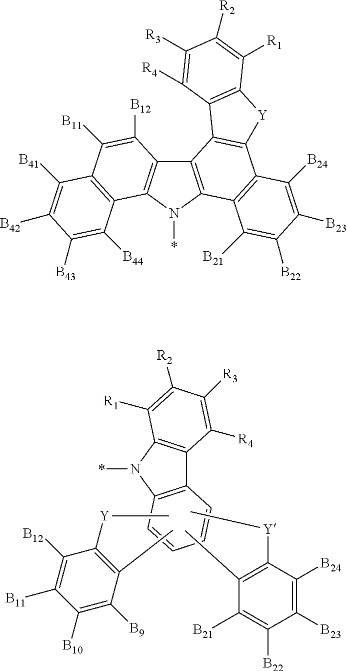

wherein
Y and Y′ each independently represent N—Ar₂, O, S, or CRₐR_{b};
Ar₂, Rₐ, R_{b}, and B₉ to B₄₄ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, or a substituted or unsubstituted (C1-C30)alkyl(C6-C30)arylamino; and
R₁ to R₈ are as defined in formula 1.

According to one embodiment of the present disclosure, B₁ to B₄₄ each independently represent hydrogen, or a substituted or unsubstituted (C6-C12)aryl, and according to another embodiment of the present disclosure, B₁ to B₄₄ each independently represent hydrogen or an unsubstituted (C6-C12)aryl. Specifically, B₁ to B₄₄ may each independently represent hydrogen or phenyl.

According to one embodiment of the present disclosure, Ar₂ each independently represent a substituted or unsubstituted (C6-C12)aryl, and according to another embodiment of the present disclosure, Ar₂ each independently represent an unsubstituted (C6-C12)aryl. Specifically, Ar₂ may represent phenyl.

According to one embodiment of the present disclosure, Rₐ and R_{b} each independently represent a substituted or unsubstituted (C1-C6)alkyl, or a substituted or unsubstituted (C6-C12)aryl, and according to another embodiment of the present disclosure, Rₐ and R_{b} each independently represent an unsubstituted (C1-C6)alkyl, or an unsubstituted (C6-C12)aryl. Specifically, Rₐ and R_{b} may each independently represent methyl or phenyl.

In formula 1, X₁ to X₃ each independently represent N or CR.

R and R₁₁ each independently represent hydrogen, deuterium, a halogen, a cyano, a substituted or unsubstituted (C1-C30)alkyl, a substituted or unsubstituted (C6-C30)aryl, or a substituted or unsubstituted (3- to 30-membered)heteroaryl.

According to one embodiment of the present disclosure, R represents hydrogen, or a substituted or unsubstituted (C6-C20)aryl, and according to another embodiment of the present disclosure, R represents hydrogen, or a (C6-C20)aryl unsubstituted or substituted with a (C1-C6)alkyl(s). Specifically, R may represent hydrogen, phenyl, biphenyl, dimethylfluorenyl, or dimethylbenzofluorenyl.

According to one embodiment of the present disclosure, R₁₁ represents hydrogen, or a substituted or unsubstituted (C6-C15)aryl, and according to another embodiment of the present disclosure, R₁₁ represents hydrogen, or an unsubstituted (C6-C15)aryl. Specifically, R₁₁ may represent hydrogen or phenyl.

L represents a single bond, a substituted or unsubstituted (C6-C30)arylene, or a substituted or unsubstituted (3- to 30-membered)heteroarylene. According to one embodiment of the present disclosure, L represents a single bond, a substituted or unsubstituted (C6-C15)arylene, or a substituted or unsubstituted (5- to 15-membered)heteroarylene, and according to another embodiment of the present disclosure, L represents a single bond, an unsubstituted (C6-C15)arylene, or an unsubstituted (5- to 15-membered)heteroarylene. Specifically, L may represent a single bond, phenylene, naphthylene, or pyridinylene.

R₁ to R₈ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, or a substituted or unsubstituted (C1-C30)alkyl(C6-C30)arylamino; or may be linked to an adjacent substituent to form a ring, with a proviso that at least one pair of R₁ to R₈ must be linked to each other to form a ring. According to one embodiment of the present disclosure, R₁ to R₈ each independently represent hydrogen, a substituted or unsubstituted (C6-C15)aryl, a substituted or unsubstituted (5- to 15-membered)heteroaryl, or a substituted or unsubstituted di(C6-C15)arylamino; or may be linked to an adjacent substituent to form a ring, and according to another embodiment of the present disclosure, R₁ to R₈ each independently represent hydrogen; an unsubstituted (C6-C15)aryl; a (5- to 15-membered)heteroaryl unsubstituted or substituted with a (C6-C12)aryl(s); or an unsubstituted di(C6-C15)arylamino; or may be linked to an adjacent substituent to form a ring. Specifically, R₁ to R₈ may each independently represent hydrogen, phenyl, biphenyl, dibenzofuranyl, dibenzothiophenyl, phenylcarbazolyl, diphenylamino, or phenylbiphenylamino, or may be linked to an adjacent substituent to form an unsubstituted benzene ring, benzofuran ring, benzothiophene ring, an indene ring substituted with a methyl(s), an indene ring substituted with a phenyl(s), an indole ring substituted with a phenyl(s), a benzoindole ring substituted with a phenyl(s), or a dibenzoindole ring substituted with a phenyl(s).

a represents an integer of 1 to 3, where a is an integer of 2 or more, each R₁₁ may be the same or different. According to one embodiment of the present disclosure, a represents 1 or 2.

According to one embodiment of the present disclosure, in formula 1 above, X₁ to X₃ each independently represent N or CR; R represents hydrogen, or a substituted or unsubstituted (C6-C20)aryl; R₁₁ represents hydrogen, or a substituted or unsubstituted (C6-C15)aryl; L represents a single bond, a substituted or unsubstituted (C6-C15)arylene, or a substituted or unsubstituted (5- to 15-membered)heteroarylene; R₁ to R₈ each independently represent hydrogen, a substituted or unsubstituted (C6-C15)aryl, a substituted or unsubstituted (5- to 15-membered)heteroaryl, or a substituted or unsubstituted di(C6-C15)arylamino; or may be linked to an adjacent substituent to form a ring, with a proviso that at least one pair of R₁ to R₈ must be linked to each other to form a ring; and a represents 1 or 2.

According to another embodiment of the present disclosure, in formula 1 above, X₁ to X₃ each independently represent N or CR; R represents hydrogen, or a (C6-C20)aryl unsubstituted or substituted with a (C1-C6)alkyl(s); R₁₁ represents hydrogen, or an unsubstituted (C6-C15)aryl; L represents a single bond, an unsubstituted (C6-C15)arylene, or an unsubstituted (5- to 15-membered)heteroarylene; R₁ to R₈ each independently represent hydrogen; an unsubstituted (C6-C15)aryl; a (5- to 15-membered)heteroaryl unsubstituted or substituted with a (C6-C12)aryl(s); or an unsubstituted di(C6-C15)arylamino; or may be linked to an adjacent substituent to form a ring, with a proviso that at least one pair of R₁ to R₈ must be linked to each other to form a ring; and a represents 1 or 2.

In formulas of the present disclosure, when a substituent is linked to an adjacent substituent to form a ring, the ring may be a substituted or unsubstituted (3- to 30-membered) mono- or polycyclic, alicyclic or aromatic ring, or the combination thereof, in which the formed ring may contain at least one heteroatom selected from nitrogen, oxygen, and sulfur.

In formulas of the present disclosure, the heteroaryl(ene) may each independently contain at least one heteroatom selected from B, N, O, S, Si, and P. In addition, the heteroatom may be substituted with at least one substituent selected from the group consisting of hydrogen, deuterium, a halogen, a cyano, a substituted or unsubstituted (C1-C30)alkyl, a substituted or unsubstituted (C6-C30)aryl, a substituted or unsubstituted (5- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, and a substituted or unsubstituted (C1-C30)alkyl(C6-C30)arylamino.

Herein, “(C1-C30)alkyl” is meant to be a linear or branched alkyl having 1 to 30 carbon atoms constituting the chain, in which the number of carbon atoms is preferably 1 to 20, more preferably 1 to 10, and includes methyl, ethyl, n-propyl, isopropyl, n-butyl, isobutyl, tert-butyl, etc. “(C2-C30)alkenyl” is meant to be a linear or branched alkenyl having 2 to 30 carbon atoms constituting the chain, in which the number of carbon atoms is preferably 2 to 20, more preferably 2 to 10, and includes vinyl, 1-propenyl, 2-propenyl, 1-butenyl, 2-butenyl, 3-butenyl, 2-methylbut-2-enyl, etc. “(C2-C30)alkynyl” is meant to be a linear or branched alkynyl having 2 to 30 carbon atoms constituting the chain, in which the number of carbon atoms is preferably 2 to 20, more preferably 2 to 10, and includes ethynyl, 1-propynyl, 2-propynyl, 1-butynyl, 2-butynyl, 3-butynyl, 1-methylpent-2-ynyl, etc. “(C3-C30)cycloalkyl” is meant to be a mono- or polycyclic hydrocarbon having 3 to 30 ring backbone carbon atoms, in which the number of carbon atoms is preferably 3 to 20, more preferably 3 to 7, and includes cyclopropyl, cyclobutyl, cyclopentyl, cyclohexyl, etc. “(3- to 7-membered)heterocycloalkyl” is meant to be a cycloalkyl having at least one heteroatom selected from the group consisting of B, N, O, S, Si, and P, preferably selected from the group consisting of O, S, and N, and 3 to 7 ring backbone atoms, preferably 5 to 7 ring backbone atoms, and includes tetrahydrofuran, pyrrolidine, thiolan, tetrahydropyran, etc. “(C6-C30)aryl(ene)” is meant to be a monocyclic or fused ring radical derived from an aromatic hydrocarbon having 6 to 30 ring backbone carbon atoms and may be partially saturated, in which the number of ring backbone carbon atoms is preferably 6 to 25, more preferably 6 to 18, may include a spiro structure, and includes phenyl, biphenyl, terphenyl, naphthyl, binaphthyl, phenylnaphthyl, naphthylphenyl, phenylterphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, etc. “(3- to 30-membered)heteroaryl(ene)” is meant to be an aryl group having at least one, preferably 1 to 4 heteroatoms selected from the group consisting of B, N, O, S, Si, and P, and 3 to 30 ring backbone atoms; may be a monocyclic ring, or a fused ring condensed with at least one benzene ring; may be partially saturated; may be one formed by linking at least one heteroaryl or aryl group to a heteroaryl group via a single bond(s); may include a spiro structure; and includes a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, benzimidazolyl, benzothiazolyl, benzoisothiazolyl, benzoisoxazolyl, benzoxazolyl, isoindolyl, indolyl, benzoindolyl, indazolyl, benzothiadiazolyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, dihydroacridinyl, etc. “Halogen” includes F, Cl, Br, and I.

Herein, “substituted” in the expression “substituted or unsubstituted” means that a hydrogen atom in a certain functional group is replaced with another atom or functional group, i.e., a substituent. The substituents of the substituted (C1-C30)alkyl, the substituted (C6-C30)aryl(ene), the substituted (3- to 30-membered)heteroaryl(ene), the substituted (C3-C30)cycloalkyl, the substituted (C1-C30)alkoxy, the substituted tri(C1-C30)alkylsilyl, the substituted di(C1-C30)alkyl(C6-C30)arylsilyl, the substituted (C1-C30)alkyldi(C6-C30)arylsilyl, the substituted tri(C6-C30)arylsilyl, the substituted mono- or di- (C1-C30)alkylamino, the substituted mono- or di- (C6-C30)arylamino, and the substituted (C1-C30)alkyl(C6-C30)arylamino in R, R₁ to R₈, R₁₁, L, Ar₂, Rₐ, R_{b}, and B₁ to B₄₄ each independently are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (5- to 30-membered)heteroaryl unsubstituted or substituted with a (C6-C30)aryl(s); a (C6-C30)aryl unsubstituted or substituted with a (5- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di- (C6-C30)arylamino unsubstituted or substituted with a (C1-C30)alkyl(s); a (C1-C30)alkyl(C6-C30)arylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl, and according to one embodiment of the present disclosure, the substituents may be each independently a (C1-C6)alkyl and/or a (C6-C12)aryl. Specifically, they may be methyl and/or phenyl.

The compound represented by formula 1 includes the following compounds, but is not limited thereto:

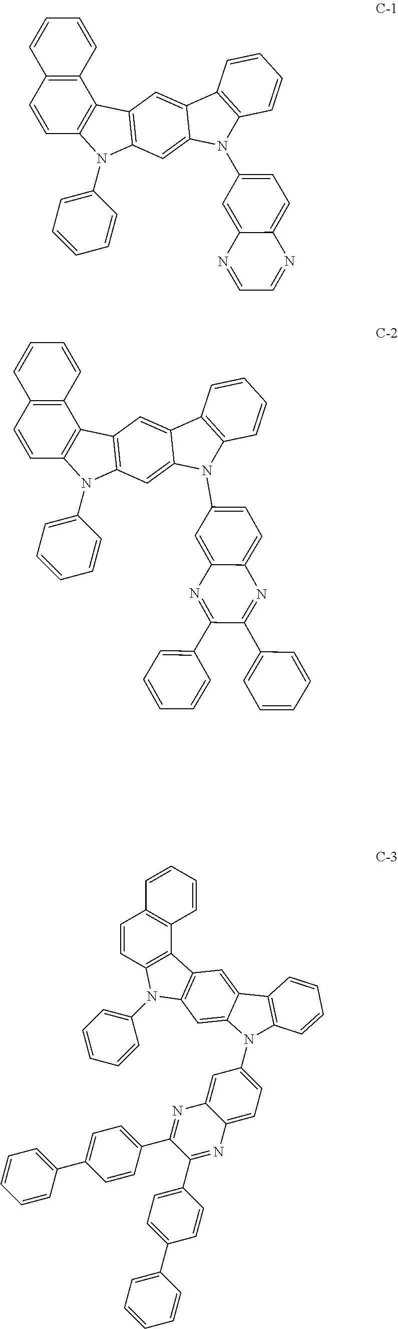 [SMILES: c1ccc(-n2c3cc4c(cc3c3c5ccccc5ccc32)c2ccccc2n4-c2ccc3nccnc3c2)cc1 ; c1ccc(-c2nc3ccc(-n4c5ccccc5c5cc6c7c8ccccc8ccc7n(-c7ccccc7)c6cc54)cc3nc2-c2ccccc2)cc1 ; c1ccc(-c2ccc(-c3nc4ccc(-n5c6ccccc6c6cc7c8c9ccccc9ccc8n(-c8ccccc8)c7cc65)cc4nc3-c3ccc(-c4ccccc4)cc3)cc2)cc1]

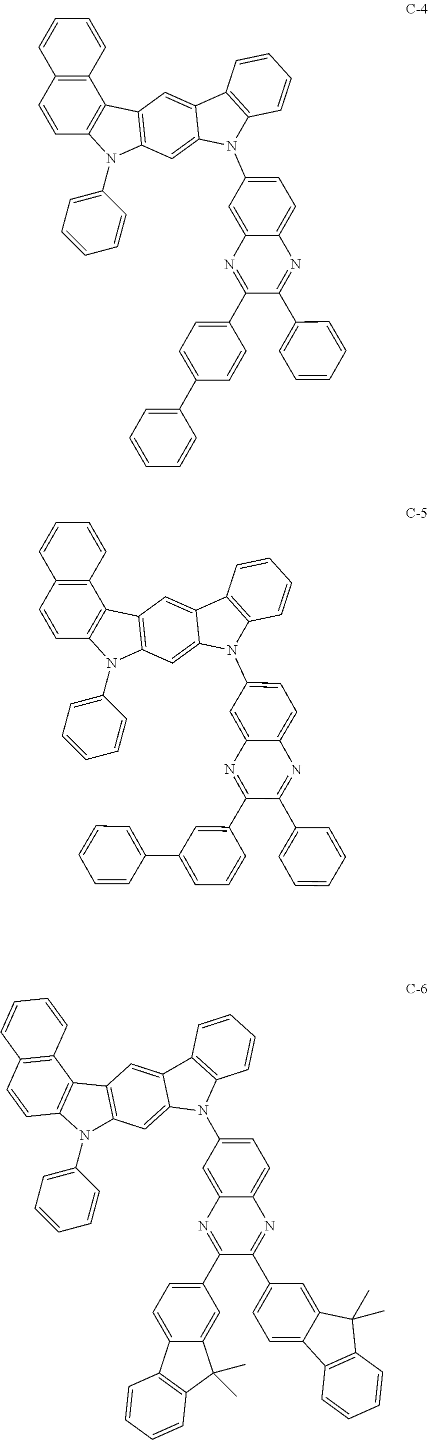 [SMILES: c1ccc(-c2ccc(-c3nc4cc(-n5c6ccccc6c6cc7c8c9ccccc9ccc8n(-c8ccccc8)c7cc65)ccc4nc3-c3ccccc3)cc2)cc1 ; c1ccc(-c2cccc(-c3nc4cc(-n5c6ccccc6c6cc7c8c9ccccc9ccc8n(-c8ccccc8)c7cc65)ccc4nc3-c3ccccc3)c2)cc1 ; CC1(C)c2ccccc2-c2ccc(-c3nc4ccc(-n5c6ccccc6c6cc7c8c9ccccc9ccc8n(-c8ccccc8)c7cc65)cc4nc3-c3ccc4c(c3)C(C)(C)c3ccccc3-4)cc21]

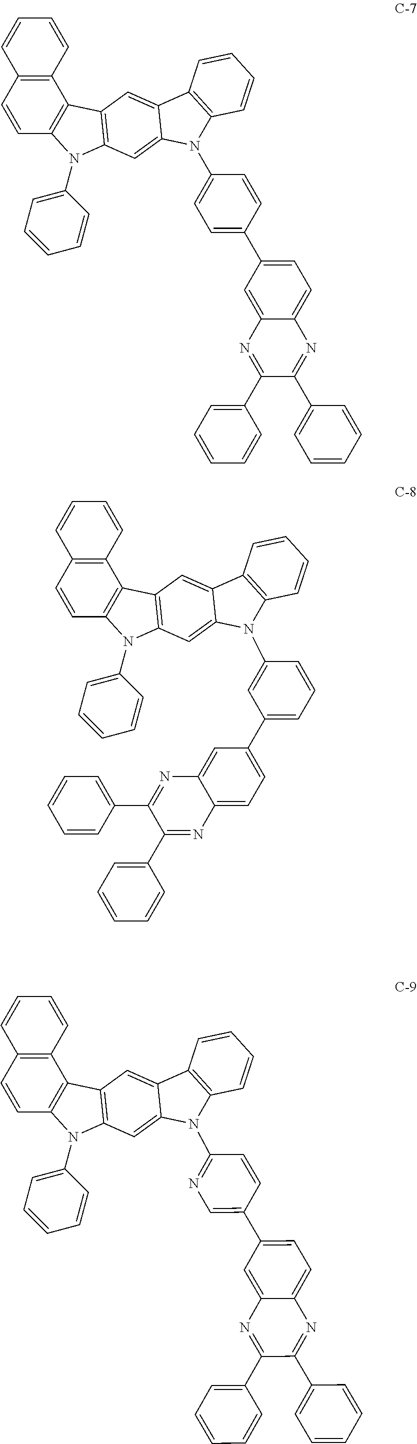 [SMILES: c1ccc(-c2nc3ccc(-c4ccc(-n5c6ccccc6c6cc7c8c9ccccc9ccc8n(-c8ccccc8)c7cc65)cc4)cc3nc2-c2ccccc2)cc1 ; c1ccc(-c2nc3ccc(-c4cccc(-n5c6ccccc6c6cc7c8c9ccccc9ccc8n(-c8ccccc8)c7cc65)c4)cc3nc2-c2ccccc2)cc1 ; c1ccc(-c2nc3ccc(-c4ccc(-n5c6ccccc6c6cc7c8c9ccccc9ccc8n(-c8ccccc8)c7cc65)nc4)cc3nc2-c2ccccc2)cc1]

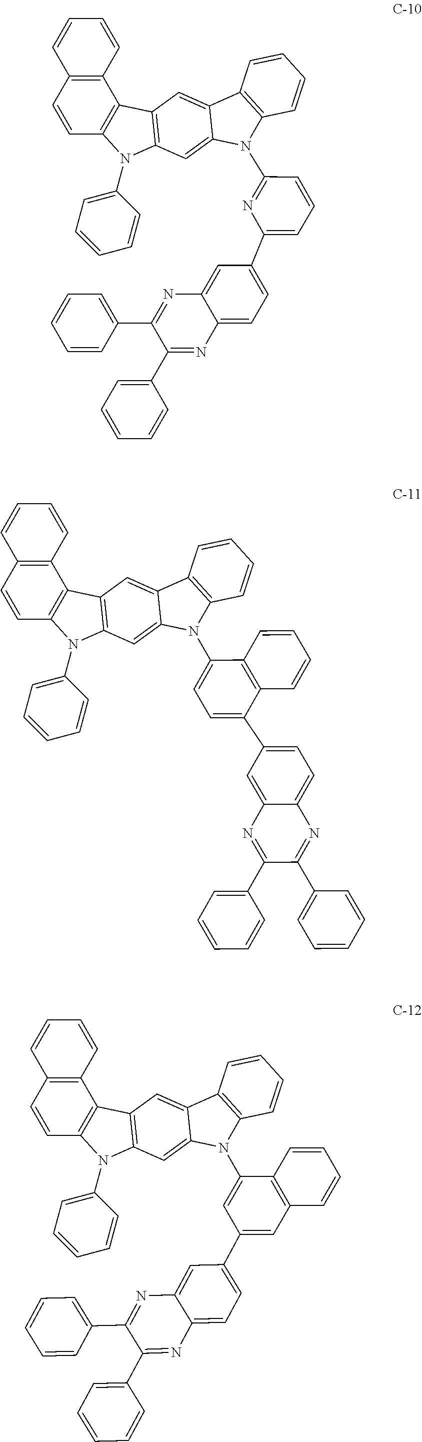 [SMILES: c1ccc(-c2nc3ccc(-c4cccc(-n5c6ccccc6c6cc7c8c9ccccc9ccc8n(-c8ccccc8)c7cc65)n4)cc3nc2-c2ccccc2)cc1 ; c1ccc(-c2nc3ccc(-c4ccc(-n5c6ccccc6c6cc7c8c9ccccc9ccc8n(-c8ccccc8)c7cc65)c5ccccc45)cc3nc2-c2ccccc2)cc1 ; c1ccc(-c2nc3ccc(-c4cc(-n5c6ccccc6c6cc7c8c9ccccc9ccc8n(-c8ccccc8)c7cc65)c5ccccc5c4)cc3nc2-c2ccccc2)cc1]

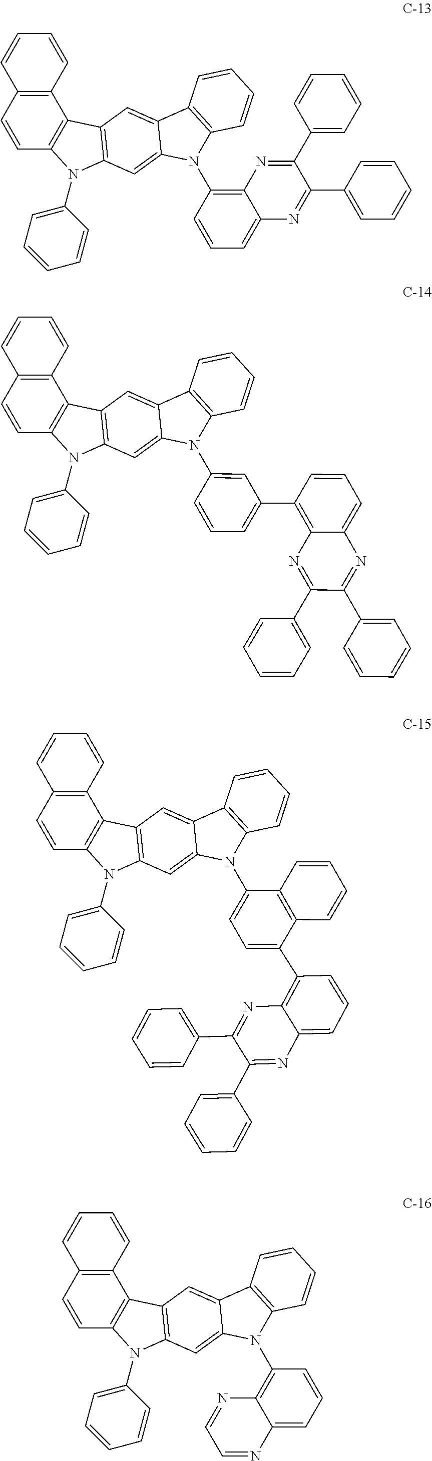 [SMILES: c1ccc(-c2nc3cccc(-n4c5ccccc5c5cc6c7c8ccccc8ccc7n(-c7ccccc7)c6cc54)c3nc2-c2ccccc2)cc1 ; c1ccc(-c2nc3cccc(-c4cccc(-n5c6ccccc6c6cc7c8c9ccccc9ccc8n(-c8ccccc8)c7cc65)c4)c3nc2-c2ccccc2)cc1 ; c1ccc(-c2nc3cccc(-c4ccc(-n5c6ccccc6c6cc7c8c9ccccc9ccc8n(-c8ccccc8)c7cc65)c5ccccc45)c3nc2-c2ccccc2)cc1 ; c1ccc(-n2c3cc4c(cc3c3c5ccccc5ccc32)c2ccccc2n4-c2cccc3nccnc23)cc1]

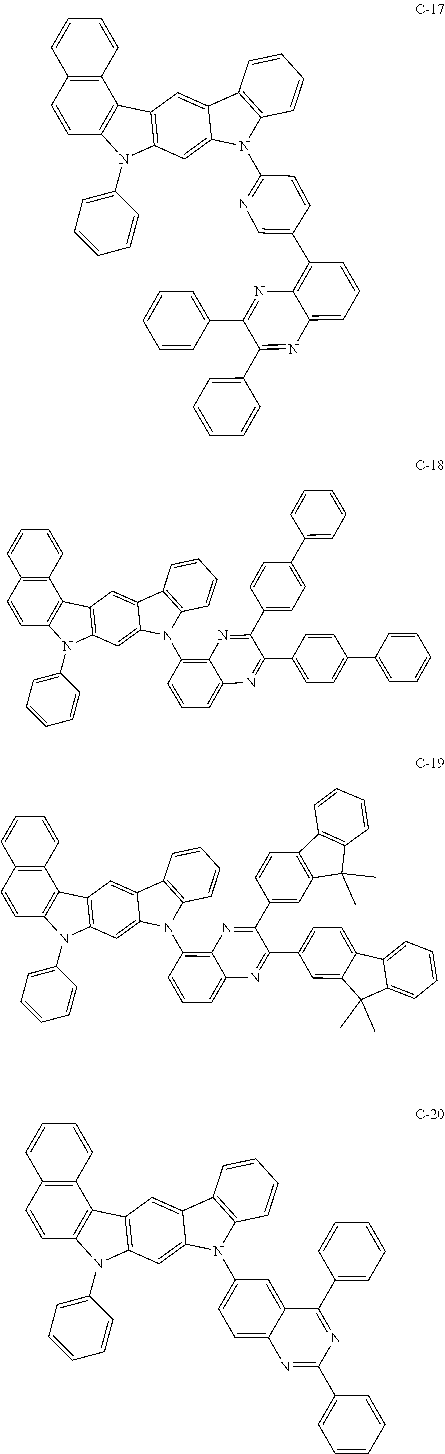 [SMILES: c1ccc(-c2nc3cccc(-c4ccc(-n5c6ccccc6c6cc7c8c9ccccc9ccc8n(-c8ccccc8)c7cc65)nc4)c3nc2-c2ccccc2)cc1 ; c1ccc(-c2ccc(-c3nc4cccc(-n5c6ccccc6c6cc7c8c9ccccc9ccc8n(-c8ccccc8)c7cc65)c4nc3-c3ccc(-c4ccccc4)cc3)cc2)cc1 ; CC1(C)c2ccccc2-c2ccc(-c3nc4cccc(-n5c6ccccc6c6cc7c8c9ccccc9ccc8n(-c8ccccc8)c7cc65)c4nc3-c3ccc4c(c3)C(C)(C)c3ccccc3-4)cc21 ; c1ccc(-c2nc(-c3ccccc3)c3cc(-n4c5ccccc5c5cc6c7c8ccccc8ccc7n(-c7ccccc7)c6cc54)ccc3n2)cc1]

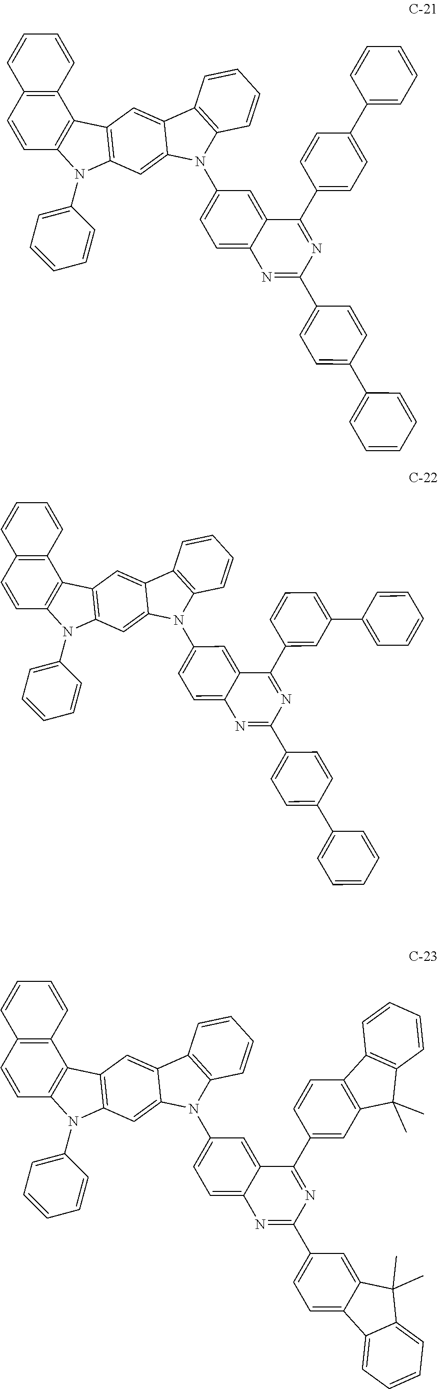 [SMILES: c1ccc(-c2ccc(-c3nc(-c4ccc(-c5ccccc5)cc4)c4cc(-n5c6ccccc6c6cc7c8c9ccccc9ccc8n(-c8ccccc8)c7cc65)ccc4n3)cc2)cc1 ; c1ccc(-c2ccc(-c3nc(-c4cccc(-c5ccccc5)c4)c4cc(-n5c6ccccc6c6cc7c8c9ccccc9ccc8n(-c8ccccc8)c7cc65)ccc4n3)cc2)cc1 ; CC1(C)c2ccccc2-c2ccc(-c3nc(-c4ccc5c(c4)C(C)(C)c4ccccc4-5)c4cc(-n5c6ccccc6c6cc7c8c9ccccc9ccc8n(-c8ccccc8)c7cc65)ccc4n3)cc21]

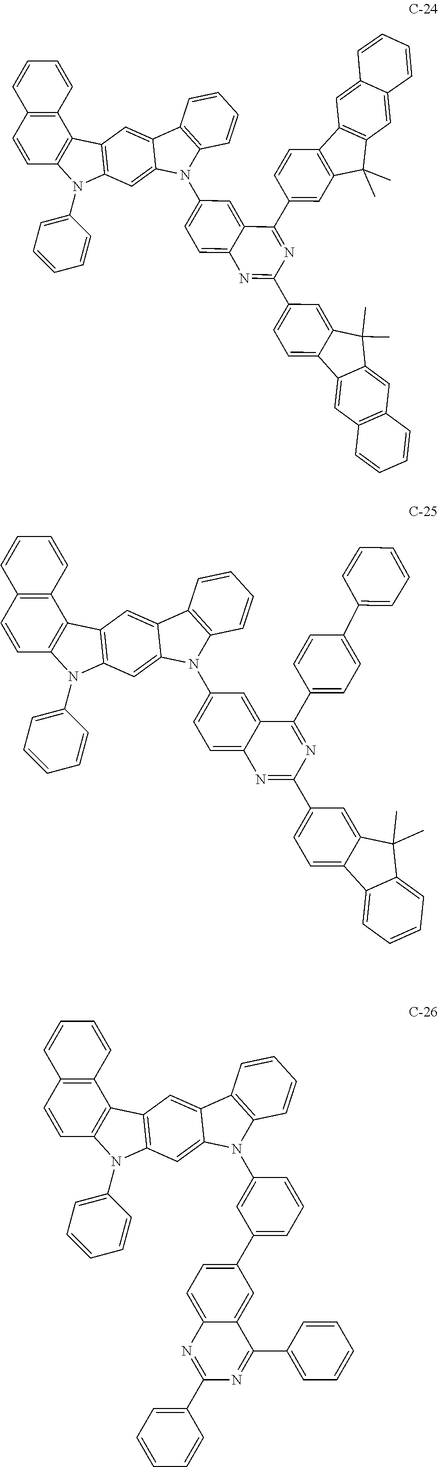 [SMILES: CC1(C)c2cc(-c3nc(-c4ccc5c(c4)C(C)(C)c4cc6ccccc6cc4-5)c4cc(-n5c6ccccc6c6cc7c8c9ccccc9ccc8n(-c8ccccc8)c7cc65)ccc4n3)ccc2-c2cc3ccccc3cc21 ; CC1(C)c2ccccc2-c2ccc(-c3nc(-c4ccc(-c5ccccc5)cc4)c4cc(-n5c6ccccc6c6cc7c8c9ccccc9ccc8n(-c8ccccc8)c7cc65)ccc4n3)cc21 ; c1ccc(-c2nc(-c3ccccc3)c3cc(-c4cccc(-n5c6ccccc6c6cc7c8c9ccccc9ccc8n(-c8ccccc8)c7cc65)c4)ccc3n2)cc1]

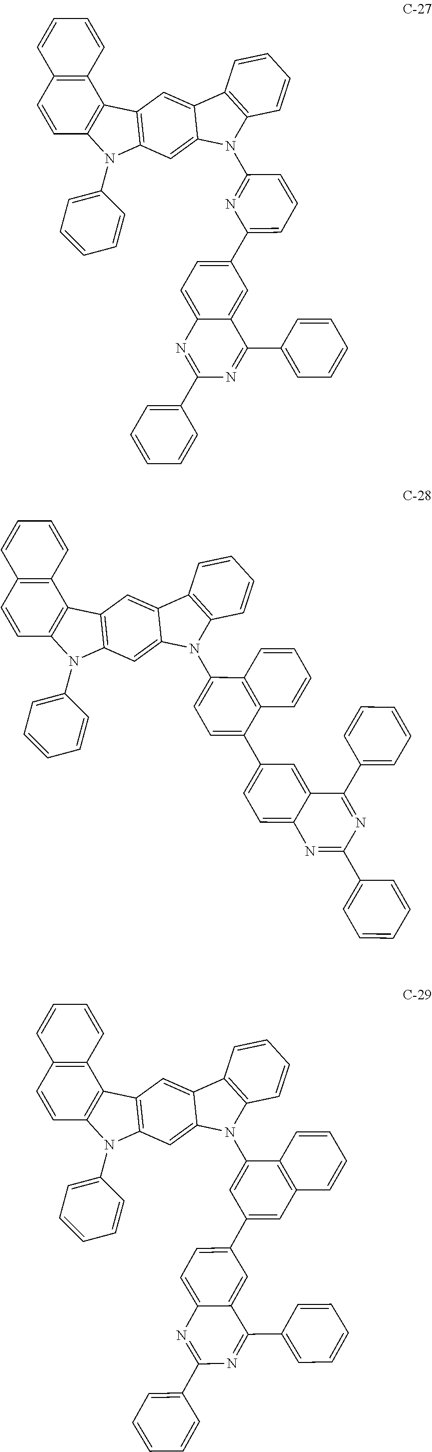 [SMILES: c1ccc(-c2nc(-c3ccccc3)c3cc(-c4cccc(-n5c6ccccc6c6cc7c8c9ccccc9ccc8n(-c8ccccc8)c7cc65)n4)ccc3n2)cc1 ; c1ccc(-c2nc(-c3ccccc3)c3cc(-c4ccc(-n5c6ccccc6c6cc7c8c9ccccc9ccc8n(-c8ccccc8)c7cc65)c5ccccc45)ccc3n2)cc1 ; c1ccc(-c2nc(-c3ccccc3)c3cc(-c4cc(-n5c6ccccc6c6cc7c8c9ccccc9ccc8n(-c8ccccc8)c7cc65)c5ccccc5c4)ccc3n2)cc1]

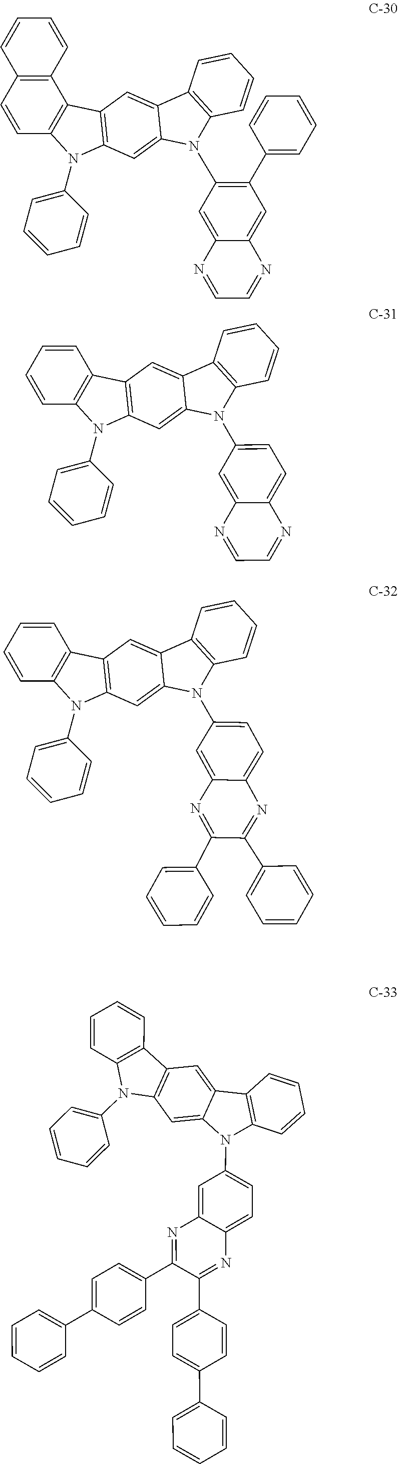

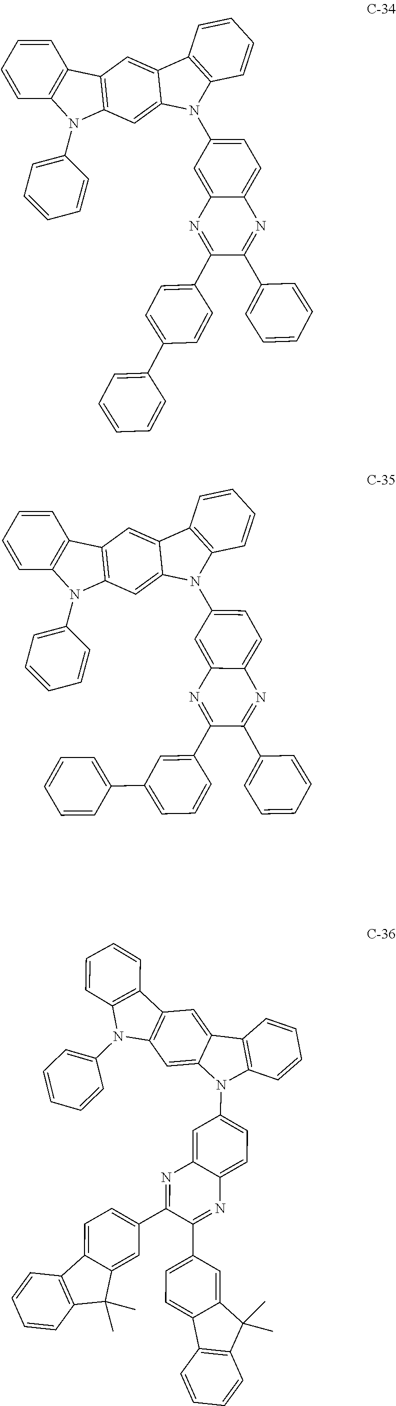 [SMILES: c1ccc(-c2ccc(-c3nc4cc(-n5c6ccccc6c6cc7c8ccccc8n(-c8ccccc8)c7cc65)ccc4nc3-c3ccccc3)cc2)cc1 ; c1ccc(-c2cccc(-c3nc4cc(-n5c6ccccc6c6cc7c8ccccc8n(-c8ccccc8)c7cc65)ccc4nc3-c3ccccc3)c2)cc1 ; CC1(C)c2ccccc2-c2ccc(-c3nc4ccc(-n5c6ccccc6c6cc7c8ccccc8n(-c8ccccc8)c7cc65)cc4nc3-c3ccc4c(c3)C(C)(C)c3ccccc3-4)cc21]

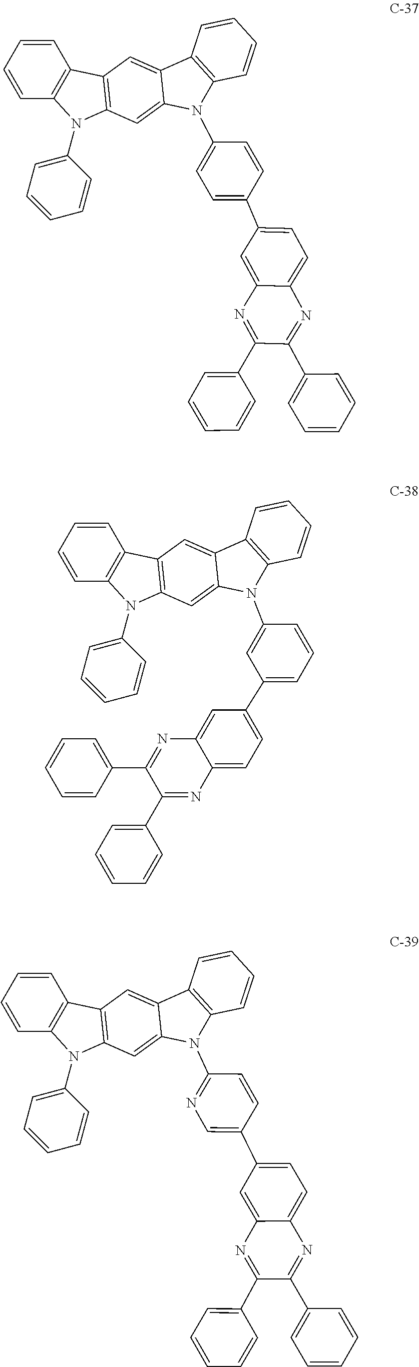 [SMILES: c1ccc(-c2nc3ccc(-c4ccc(-n5c6ccccc6c6cc7c8ccccc8n(-c8ccccc8)c7cc65)cc4)cc3nc2-c2ccccc2)cc1 ; c1ccc(-c2nc3ccc(-c4cccc(-n5c6ccccc6c6cc7c8ccccc8n(-c8ccccc8)c7cc65)c4)cc3nc2-c2ccccc2)cc1 ; c1ccc(-c2nc3ccc(-c4ccc(-n5c6ccccc6c6cc7c8ccccc8n(-c8ccccc8)c7cc65)nc4)cc3nc2-c2ccccc2)cc1]

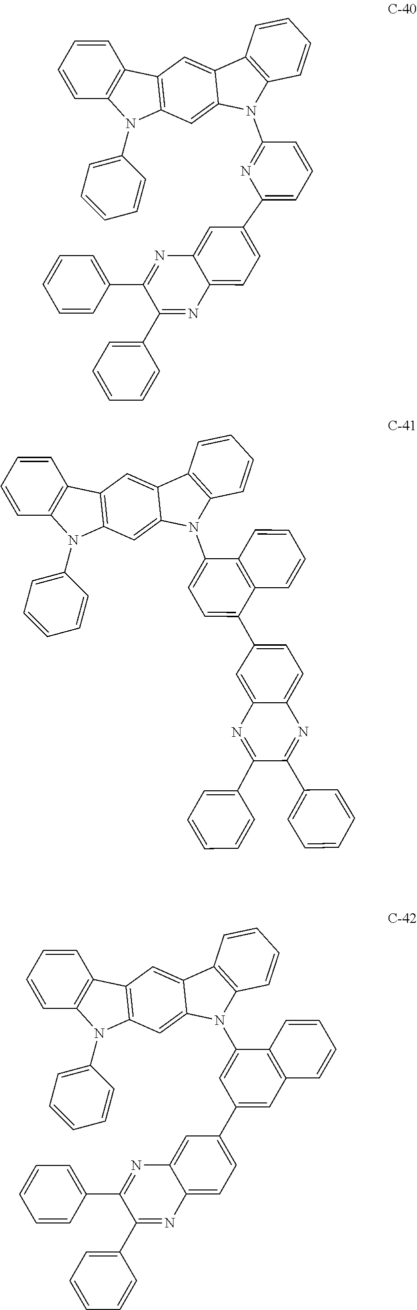 [SMILES: c1ccc(-c2nc3ccc(-c4cccc(-n5c6ccccc6c6cc7c8ccccc8n(-c8ccccc8)c7cc65)n4)cc3nc2-c2ccccc2)cc1 ; c1ccc(-c2nc3ccc(-c4ccc(-n5c6ccccc6c6cc7c8ccccc8n(-c8ccccc8)c7cc65)c5ccccc45)cc3nc2-c2ccccc2)cc1 ; c1ccc(-c2nc3ccc(-c4cc(-n5c6ccccc6c6cc7c8ccccc8n(-c8ccccc8)c7cc65)c5ccccc5c4)cc3nc2-c2ccccc2)cc1]

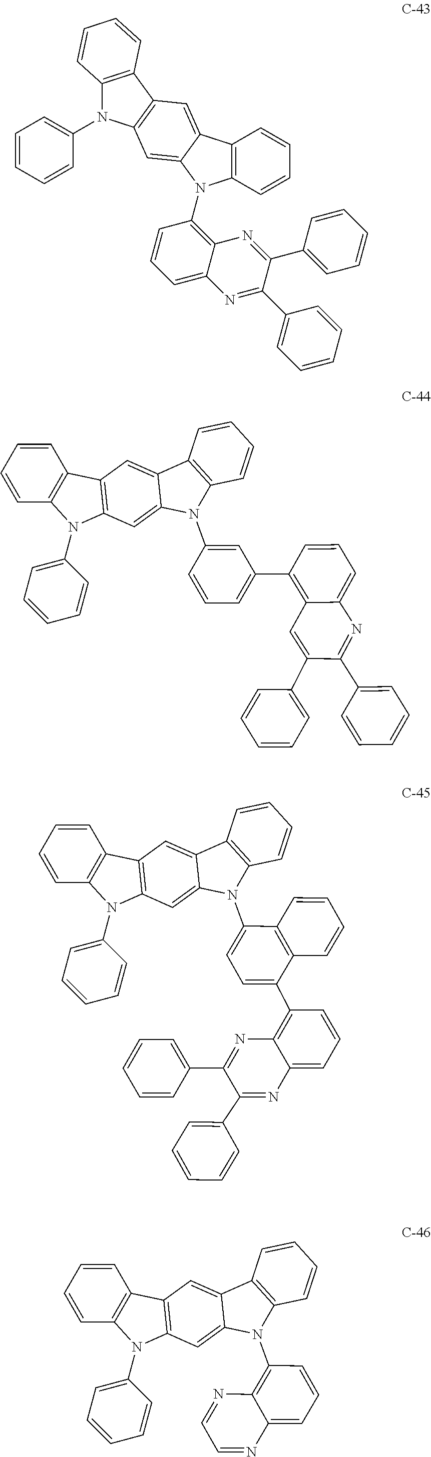 [SMILES: c1ccc(-c2nc3cccc(-n4c5ccccc5c5cc6c7ccccc7n(-c7ccccc7)c6cc54)c3nc2-c2ccccc2)cc1 ; c1ccc(-c2cc3c(-c4cccc(-n5c6ccccc6c6cc7c8ccccc8n(-c8ccccc8)c7cc65)c4)cccc3nc2-c2ccccc2)cc1 ; c1ccc(-c2nc3cccc(-c4ccc(-n5c6ccccc6c6cc7c8ccccc8n(-c8ccccc8)c7cc65)c5ccccc45)c3nc2-c2ccccc2)cc1 ; c1ccc(-n2c3ccccc3c3cc4c5ccccc5n(-c5cccc6nccnc56)c4cc32)cc1]

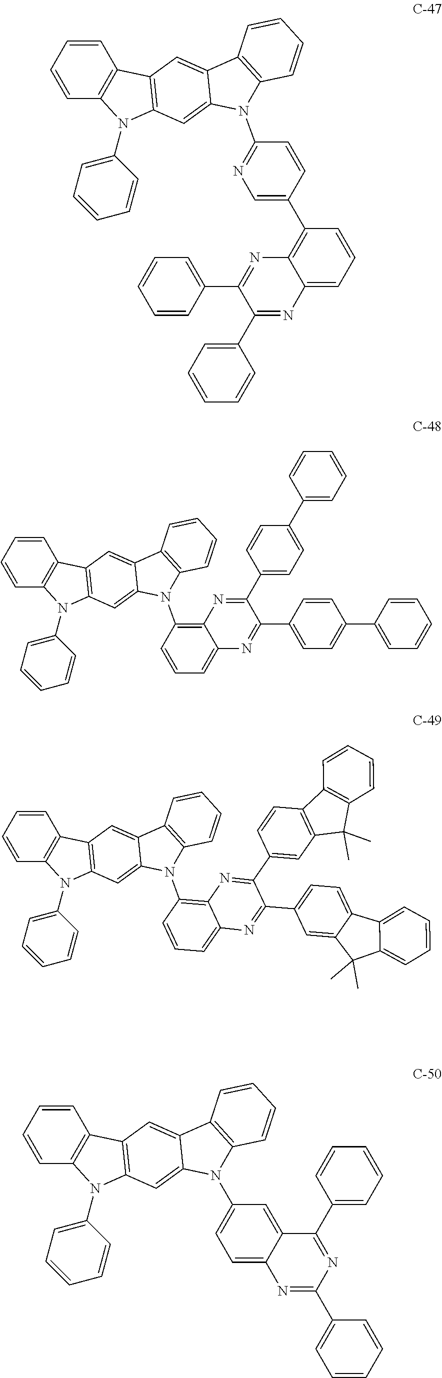 [SMILES: c1ccc(-c2nc3cccc(-c4ccc(-n5c6ccccc6c6cc7c8ccccc8n(-c8ccccc8)c7cc65)nc4)c3nc2-c2ccccc2)cc1 ; c1ccc(-c2ccc(-c3nc4cccc(-n5c6ccccc6c6cc7c8ccccc8n(-c8ccccc8)c7cc65)c4nc3-c3ccc(-c4ccccc4)cc3)cc2)cc1 ; CC1(C)c2ccccc2-c2ccc(-c3nc4cccc(-n5c6ccccc6c6cc7c8ccccc8n(-c8ccccc8)c7cc65)c4nc3-c3ccc4c(c3)C(C)(C)c3ccccc3-4)cc21 ; c1ccc(-c2nc(-c3ccccc3)c3cc(-n4c5ccccc5c5cc6c7ccccc7n(-c7ccccc7)c6cc54)ccc3n2)cc1]

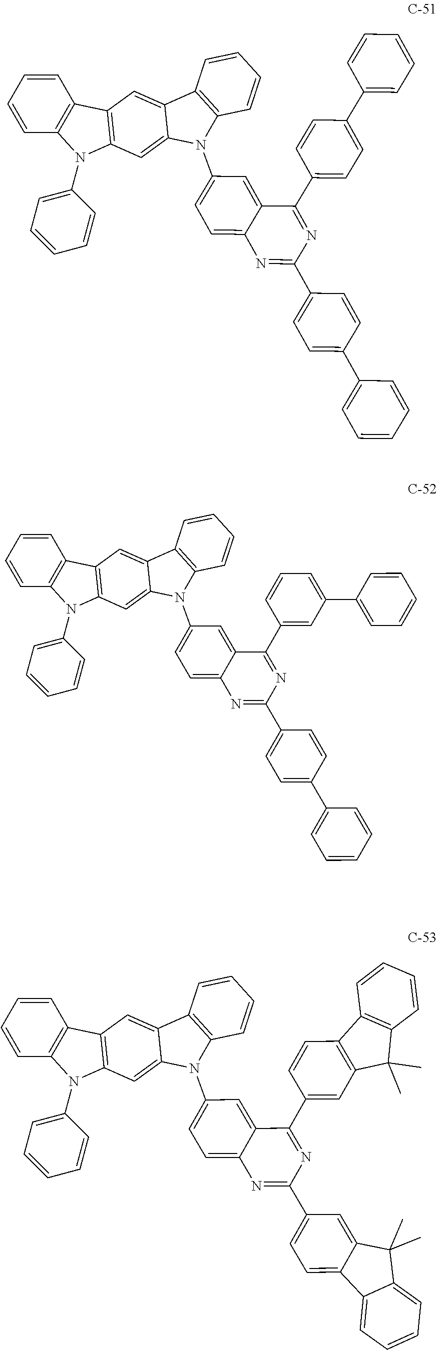 [SMILES: c1ccc(-c2ccc(-c3nc(-c4ccc(-c5ccccc5)cc4)c4cc(-n5c6ccccc6c6cc7c8ccccc8n(-c8ccccc8)c7cc65)ccc4n3)cc2)cc1 ; c1ccc(-c2ccc(-c3nc(-c4cccc(-c5ccccc5)c4)c4cc(-n5c6ccccc6c6cc7c8ccccc8n(-c8ccccc8)c7cc65)ccc4n3)cc2)cc1 ; CC1(C)c2ccccc2-c2ccc(-c3nc(-c4ccc5c(c4)C(C)(C)c4ccccc4-5)c4cc(-n5c6ccccc6c6cc7c8ccccc8n(-c8ccccc8)c7cc65)ccc4n3)cc21]

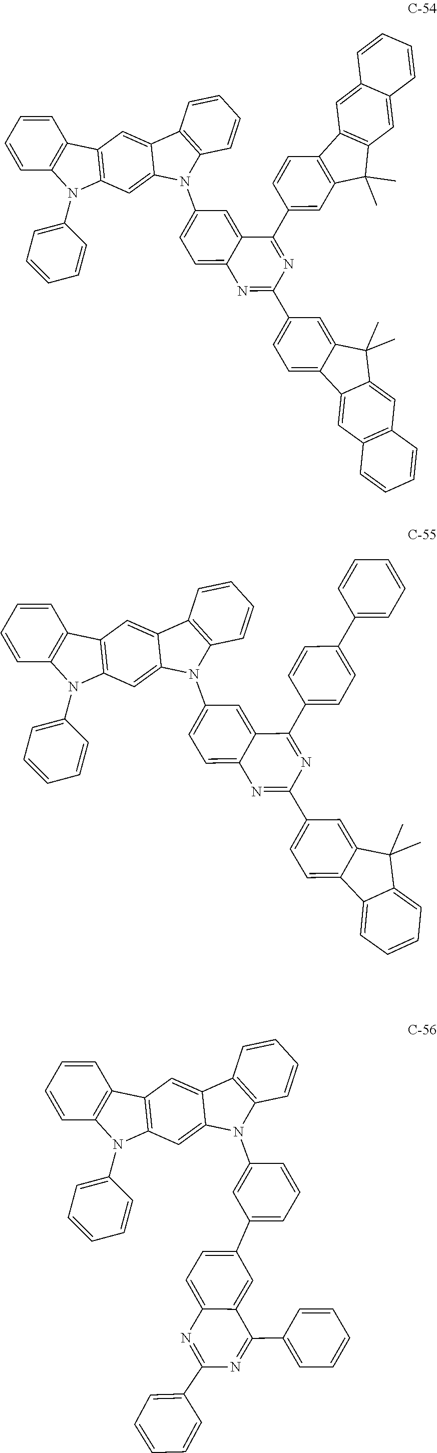 [SMILES: CC1(C)c2cc(-c3nc(-c4ccc5c(c4)C(C)(C)c4cc6ccccc6cc4-5)c4cc(-n5c6ccccc6c6cc7c8ccccc8n(-c8ccccc8)c7cc65)ccc4n3)ccc2-c2cc3ccccc3cc21 ; CC1(C)c2ccccc2-c2ccc(-c3nc(-c4ccc(-c5ccccc5)cc4)c4cc(-n5c6ccccc6c6cc7c8ccccc8n(-c8ccccc8)c7cc65)ccc4n3)cc21 ; c1ccc(-c2nc(-c3ccccc3)c3cc(-c4cccc(-n5c6ccccc6c6cc7c8ccccc8n(-c8ccccc8)c7cc65)c4)ccc3n2)cc1]

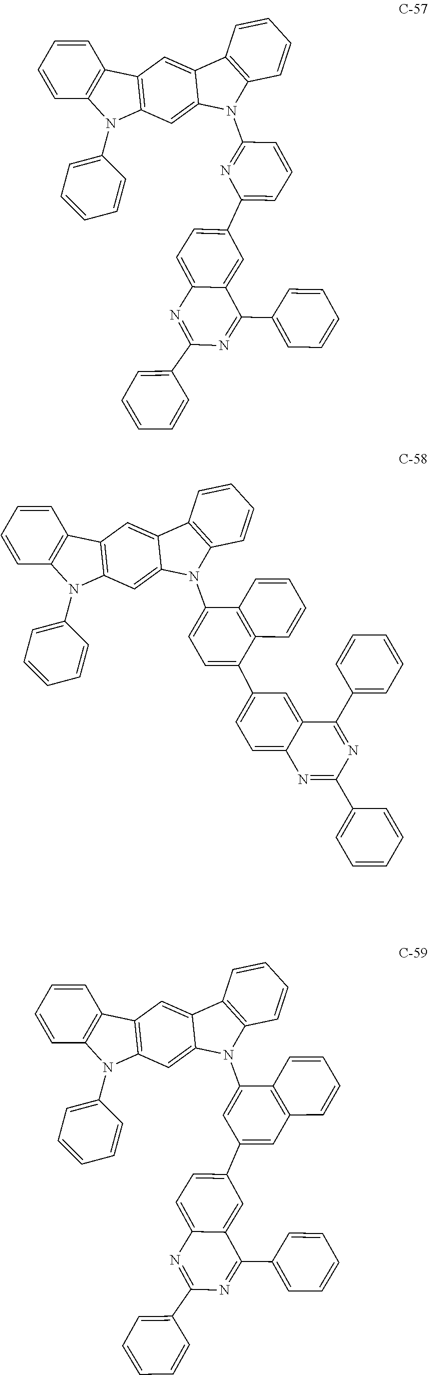 [SMILES: c1ccc(-c2nc(-c3ccccc3)c3cc(-c4cccc(-n5c6ccccc6c6cc7c8ccccc8n(-c8ccccc8)c7cc65)n4)ccc3n2)cc1 ; c1ccc(-c2nc(-c3ccccc3)c3cc(-c4ccc(-n5c6ccccc6c6cc7c8ccccc8n(-c8ccccc8)c7cc65)c5ccccc45)ccc3n2)cc1 ; c1ccc(-c2nc(-c3ccccc3)c3cc(-c4cc(-n5c6ccccc6c6cc7c8ccccc8n(-c8ccccc8)c7cc65)c5ccccc5c4)ccc3n2)cc1]

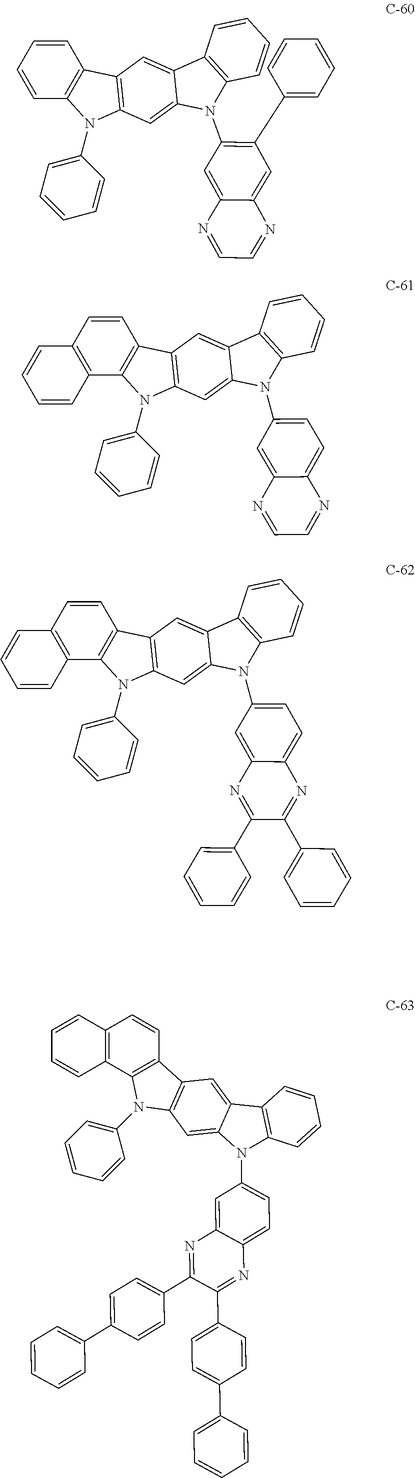

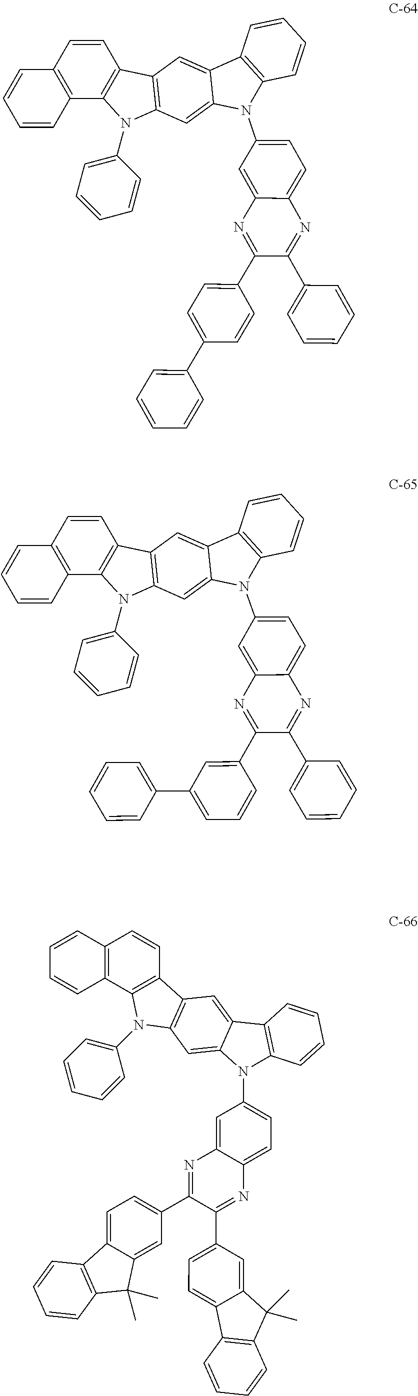 [SMILES: c1ccc(-c2ccc(-c3nc4cc(-n5c6ccccc6c6cc7c8ccc9ccccc9c8n(-c8ccccc8)c7cc65)ccc4nc3-c3ccccc3)cc2)cc1 ; c1ccc(-c2cccc(-c3nc4cc(-n5c6ccccc6c6cc7c8ccc9ccccc9c8n(-c8ccccc8)c7cc65)ccc4nc3-c3ccccc3)c2)cc1 ; CC1(C)c2ccccc2-c2ccc(-c3nc4ccc(-n5c6ccccc6c6cc7c8ccc9ccccc9c8n(-c8ccccc8)c7cc65)cc4nc3-c3ccc4c(c3)C(C)(C)c3ccccc3-4)cc21]

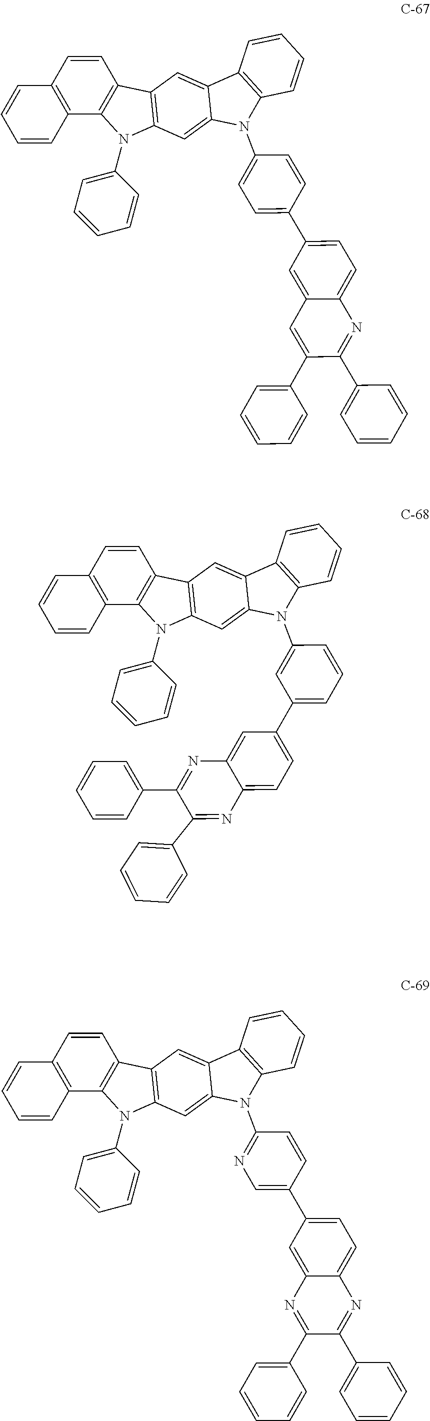 [SMILES: c1ccc(-c2cc3cc(-c4ccc(-n5c6ccccc6c6cc7c8ccc9ccccc9c8n(-c8ccccc8)c7cc65)cc4)ccc3nc2-c2ccccc2)cc1 ; c1ccc(-c2nc3ccc(-c4cccc(-n5c6ccccc6c6cc7c8ccc9ccccc9c8n(-c8ccccc8)c7cc65)c4)cc3nc2-c2ccccc2)cc1 ; c1ccc(-c2nc3ccc(-c4ccc(-n5c6ccccc6c6cc7c8ccc9ccccc9c8n(-c8ccccc8)c7cc65)nc4)cc3nc2-c2ccccc2)cc1]

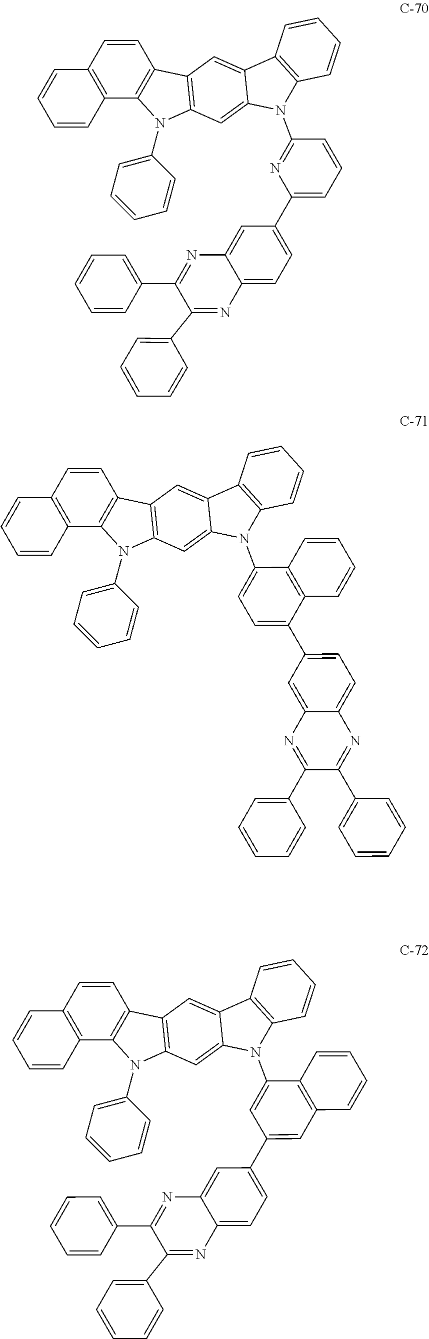 [SMILES: c1ccc(-c2nc3ccc(-c4cccc(-n5c6ccccc6c6cc7c8ccc9ccccc9c8n(-c8ccccc8)c7cc65)n4)cc3nc2-c2ccccc2)cc1 ; c1ccc(-c2nc3ccc(-c4ccc(-n5c6ccccc6c6cc7c8ccc9ccccc9c8n(-c8ccccc8)c7cc65)c5ccccc45)cc3nc2-c2ccccc2)cc1 ; c1ccc(-c2nc3ccc(-c4cc(-n5c6ccccc6c6cc7c8ccc9ccccc9c8n(-c8ccccc8)c7cc65)c5ccccc5c4)cc3nc2-c2ccccc2)cc1]

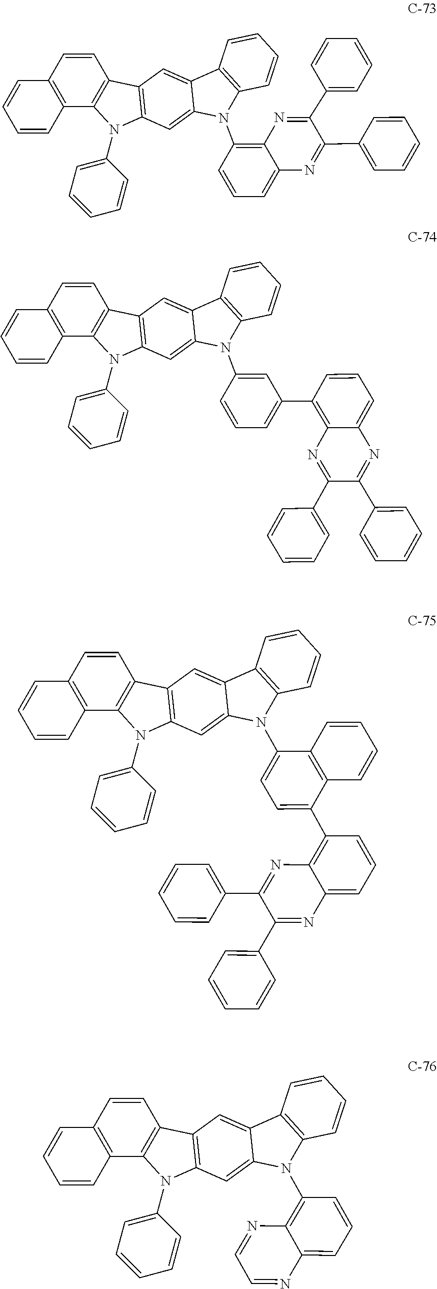 [SMILES: c1ccc(-c2nc3cccc(-n4c5ccccc5c5cc6c7ccc8ccccc8c7n(-c7ccccc7)c6cc54)c3nc2-c2ccccc2)cc1 ; c1ccc(-c2nc3cccc(-c4cccc(-n5c6ccccc6c6cc7c8ccc9ccccc9c8n(-c8ccccc8)c7cc65)c4)c3nc2-c2ccccc2)cc1 ; c1ccc(-c2nc3cccc(-c4ccc(-n5c6ccccc6c6cc7c8ccc9ccccc9c8n(-c8ccccc8)c7cc65)c5ccccc45)c3nc2-c2ccccc2)cc1 ; c1ccc(-n2c3cc4c(cc3c3ccc5ccccc5c32)c2ccccc2n4-c2cccc3nccnc23)cc1]

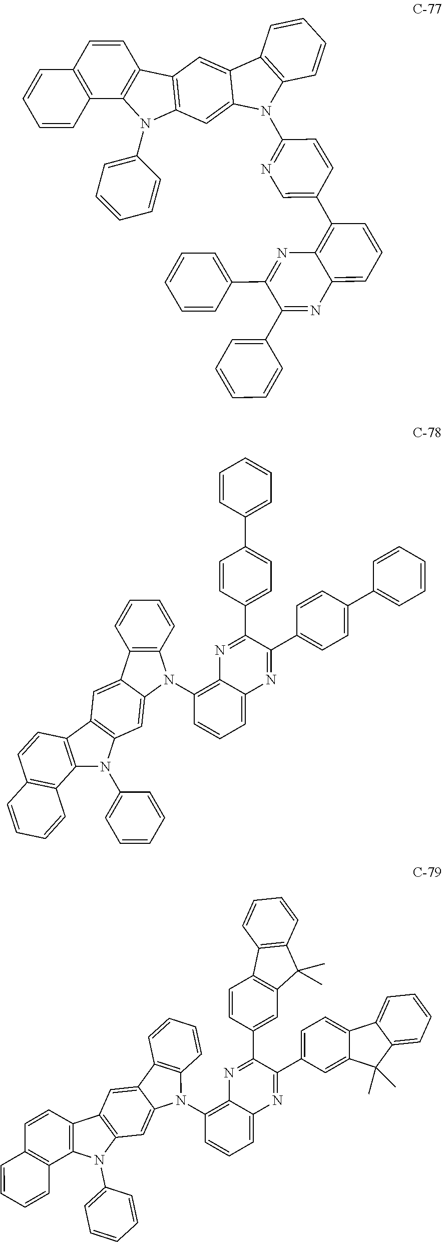 [SMILES: c1ccc(-c2nc3cccc(-c4ccc(-n5c6ccccc6c6cc7c8ccc9ccccc9c8n(-c8ccccc8)c7cc65)nc4)c3nc2-c2ccccc2)cc1 ; c1ccc(-c2ccc(-c3nc4cccc(-n5c6ccccc6c6cc7c8ccc9ccccc9c8n(-c8ccccc8)c7cc65)c4nc3-c3ccc(-c4ccccc4)cc3)cc2)cc1 ; CC1(C)c2ccccc2-c2ccc(-c3nc4cccc(-n5c6ccccc6c6cc7c8ccc9ccccc9c8n(-c8ccccc8)c7cc65)c4nc3-c3ccc4c(c3)C(C)(C)c3ccccc3-4)cc21]

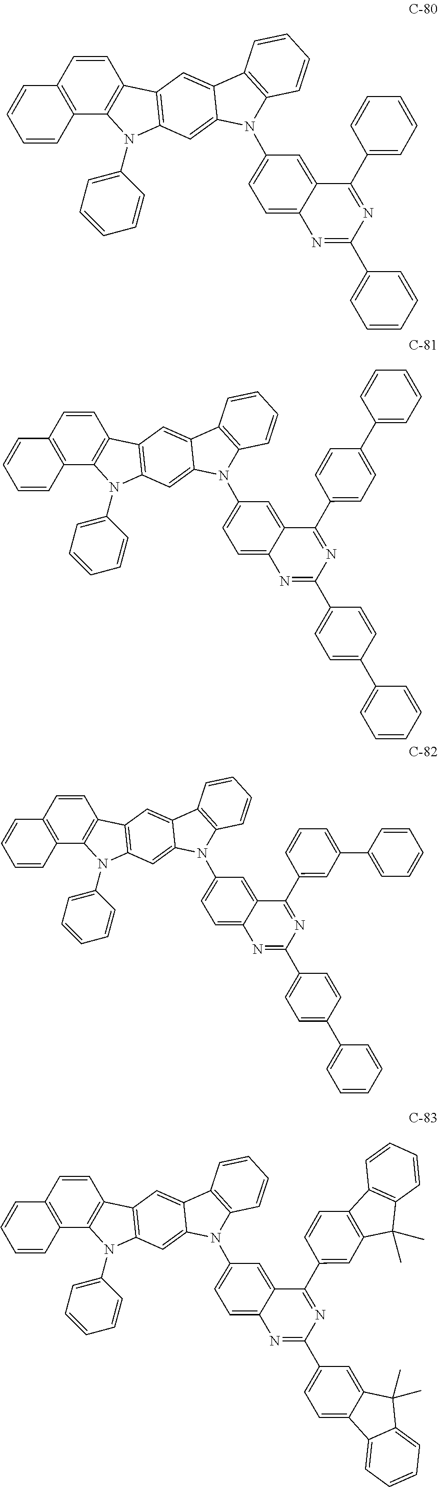

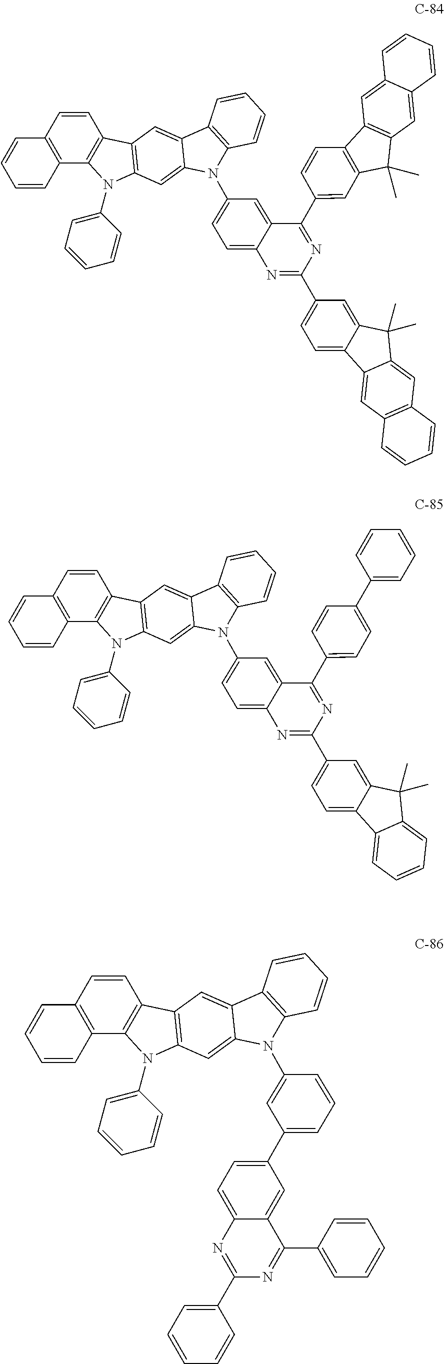 [SMILES: CC1(C)c2cc(-c3nc(-c4ccc5c(c4)C(C)(C)c4cc6ccccc6cc4-5)c4cc(-n5c6ccccc6c6cc7c8ccc9ccccc9c8n(-c8ccccc8)c7cc65)ccc4n3)ccc2-c2cc3ccccc3cc21 ; CC1(C)c2ccccc2-c2ccc(-c3nc(-c4ccc(-c5ccccc5)cc4)c4cc(-n5c6ccccc6c6cc7c8ccc9ccccc9c8n(-c8ccccc8)c7cc65)ccc4n3)cc21 ; c1ccc(-c2nc(-c3ccccc3)c3cc(-c4cccc(-n5c6ccccc6c6cc7c8ccc9ccccc9c8n(-c8ccccc8)c7cc65)c4)ccc3n2)cc1]

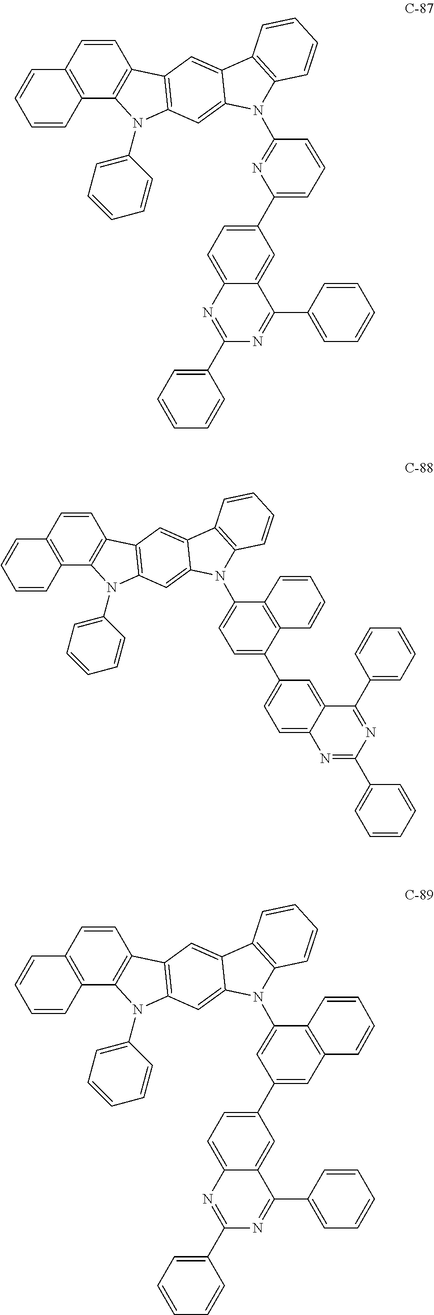 [SMILES: c1ccc(-c2nc(-c3ccccc3)c3cc(-c4cccc(-n5c6ccccc6c6cc7c8ccc9ccccc9c8n(-c8ccccc8)c7cc65)n4)ccc3n2)cc1 ; c1ccc(-c2nc(-c3ccccc3)c3cc(-c4ccc(-n5c6ccccc6c6cc7c8ccc9ccccc9c8n(-c8ccccc8)c7cc65)c5ccccc45)ccc3n2)cc1 ; c1ccc(-c2nc(-c3ccccc3)c3cc(-c4cc(-n5c6ccccc6c6cc7c8ccc9ccccc9c8n(-c8ccccc8)c7cc65)c5ccccc5c4)ccc3n2)cc1]

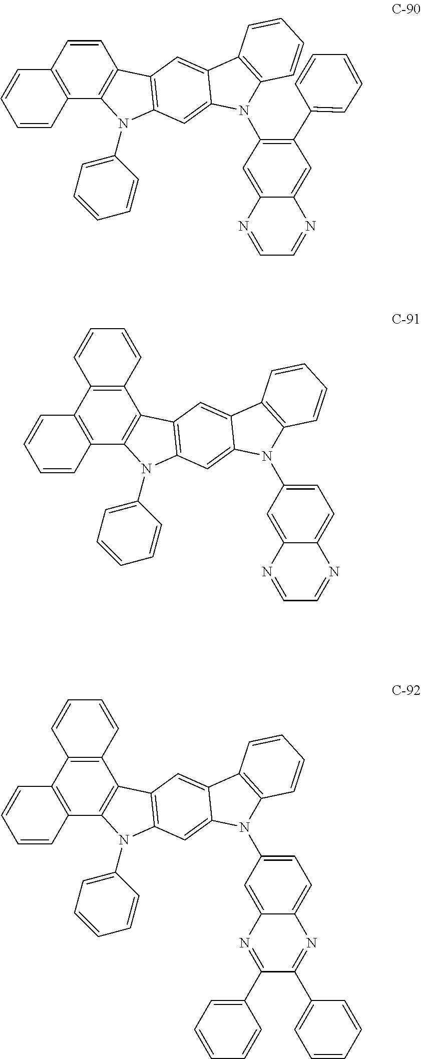 [SMILES: c1ccc(-c2cc3nccnc3cc2-n2c3ccccc3c3cc4c5ccc6ccccc6c5n(-c5ccccc5)c4cc32)cc1 ; c1ccc(-n2c3cc4c(cc3c3c5ccccc5c5ccccc5c32)c2ccccc2n4-c2ccc3nccnc3c2)cc1 ; c1ccc(-c2nc3ccc(-n4c5ccccc5c5cc6c7c8ccccc8c8ccccc8c7n(-c7ccccc7)c6cc54)cc3nc2-c2ccccc2)cc1]

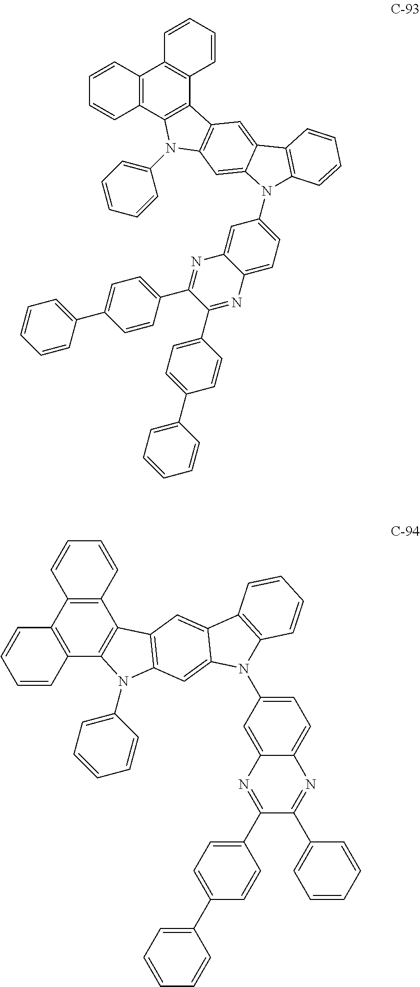 [SMILES: c1ccc(-c2ccc(-c3nc4ccc(-n5c6ccccc6c6cc7c8c9ccccc9c9ccccc9c8n(-c8ccccc8)c7cc65)cc4nc3-c3ccc(-c4ccccc4)cc3)cc2)cc1 ; c1ccc(-c2ccc(-c3nc4cc(-n5c6ccccc6c6cc7c8c9ccccc9c9ccccc9c8n(-c8ccccc8)c7cc65)ccc4nc3-c3ccccc3)cc2)cc1]

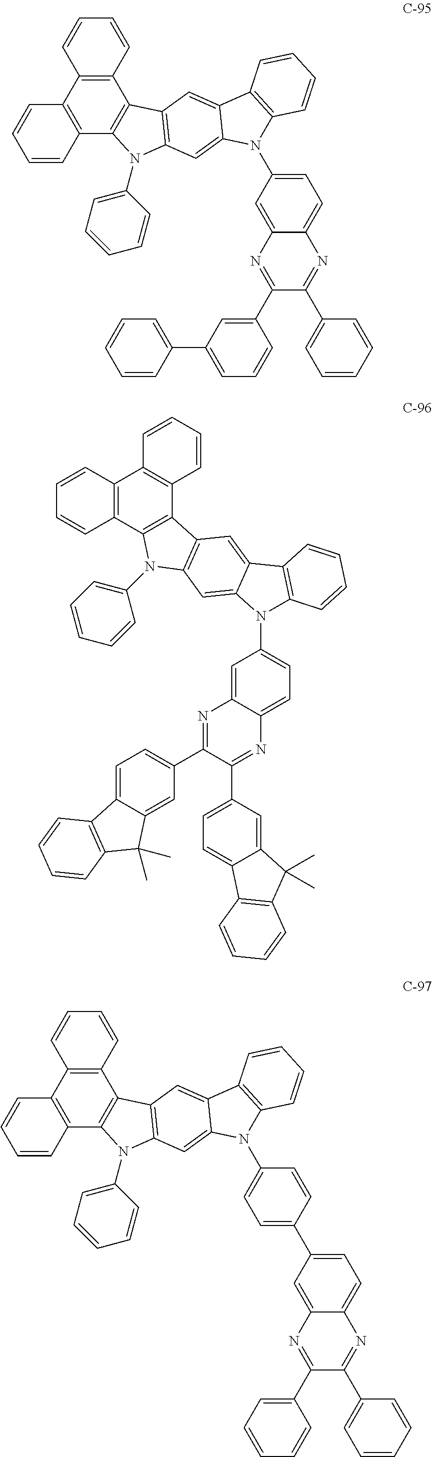

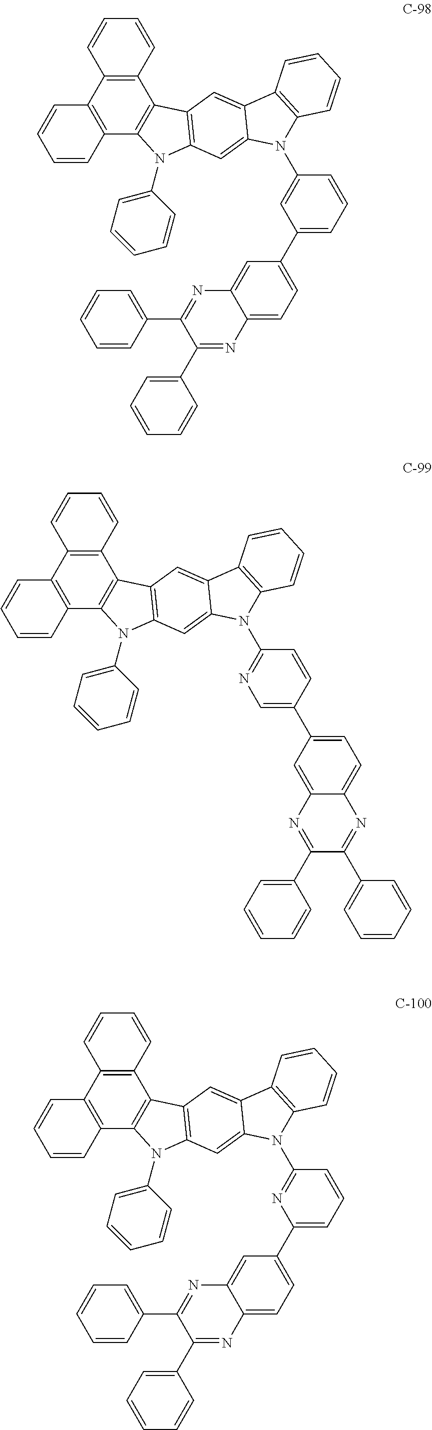 [SMILES: c1ccc(-c2nc3ccc(-c4cccc(-n5c6ccccc6c6cc7c8c9ccccc9c9ccccc9c8n(-c8ccccc8)c7cc65)c4)cc3nc2-c2ccccc2)cc1 ; c1ccc(-c2nc3ccc(-c4ccc(-n5c6ccccc6c6cc7c8c9ccccc9c9ccccc9c8n(-c8ccccc8)c7cc65)nc4)cc3nc2-c2ccccc2)cc1 ; c1ccc(-c2nc3ccc(-c4cccc(-n5c6ccccc6c6cc7c8c9ccccc9c9ccccc9c8n(-c8ccccc8)c7cc65)n4)cc3nc2-c2ccccc2)cc1]

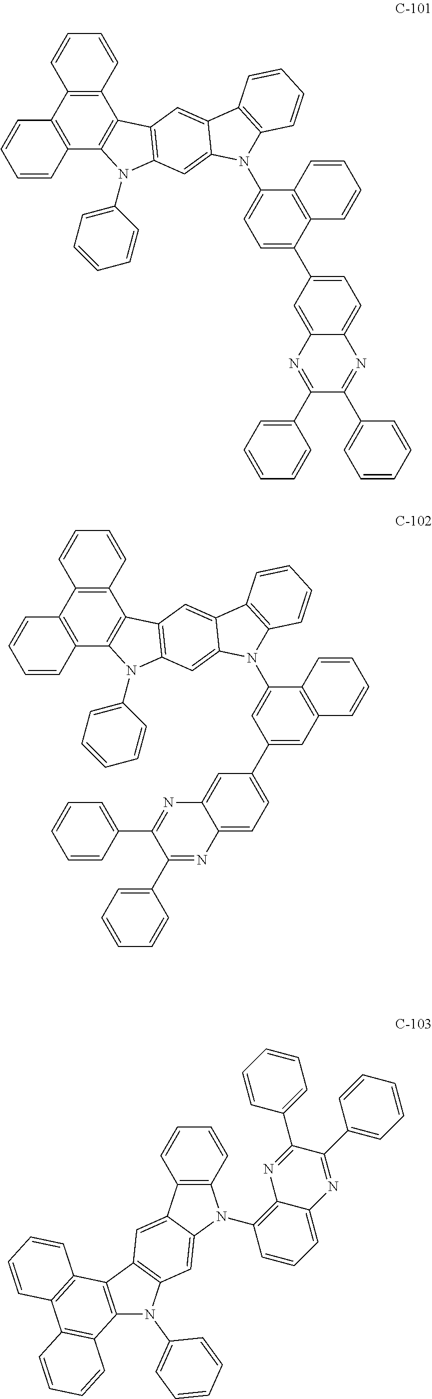 [SMILES: c1ccc(-c2nc3ccc(-c4ccc(-n5c6ccccc6c6cc7c8c9ccccc9c9ccccc9c8n(-c8ccccc8)c7cc65)c5ccccc45)cc3nc2-c2ccccc2)cc1 ; c1ccc(-c2nc3ccc(-c4cc(-n5c6ccccc6c6cc7c8c9ccccc9c9ccccc9c8n(-c8ccccc8)c7cc65)c5ccccc5c4)cc3nc2-c2ccccc2)cc1 ; c1ccc(-c2nc3cccc(-n4c5ccccc5c5cc6c7c8ccccc8c8ccccc8c7n(-c7ccccc7)c6cc54)c3nc2-c2ccccc2)cc1]

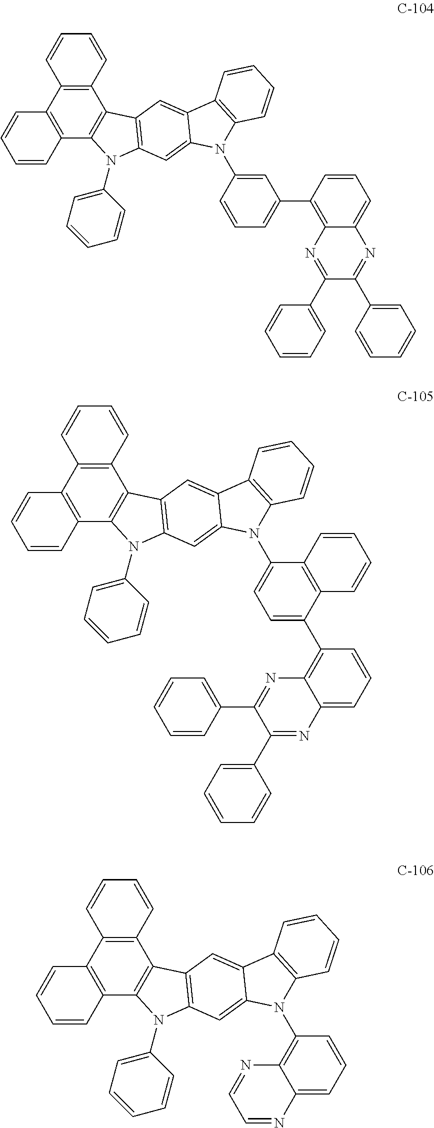 [SMILES: c1ccc(-c2nc3cccc(-c4cccc(-n5c6ccccc6c6cc7c8c9ccccc9c9ccccc9c8n(-c8ccccc8)c7cc65)c4)c3nc2-c2ccccc2)cc1 ; c1ccc(-c2nc3cccc(-c4ccc(-n5c6ccccc6c6cc7c8c9ccccc9c9ccccc9c8n(-c8ccccc8)c7cc65)c5ccccc45)c3nc2-c2ccccc2)cc1 ; c1ccc(-n2c3cc4c(cc3c3c5ccccc5c5ccccc5c32)c2ccccc2n4-c2cccc3nccnc23)cc1]

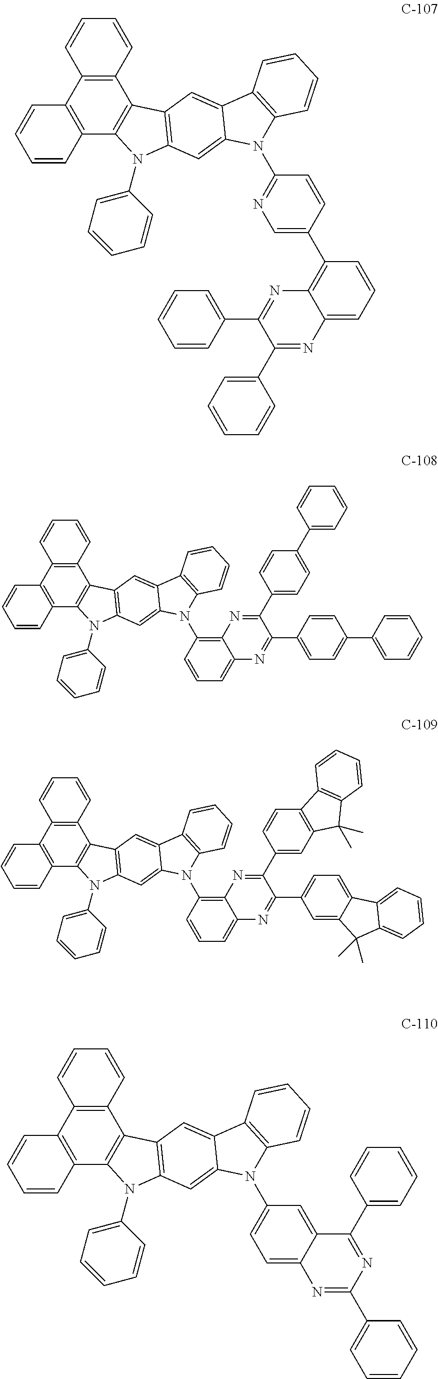 [SMILES: c1ccc(-c2nc3cccc(-c4ccc(-n5c6ccccc6c6cc7c8c9ccccc9c9ccccc9c8n(-c8ccccc8)c7cc65)nc4)c3nc2-c2ccccc2)cc1 ; c1ccc(-c2ccc(-c3nc4cccc(-n5c6ccccc6c6cc7c8c9ccccc9c9ccccc9c8n(-c8ccccc8)c7cc65)c4nc3-c3ccc(-c4ccccc4)cc3)cc2)cc1 ; CC1(C)c2ccccc2-c2ccc(-c3nc4cccc(-n5c6ccccc6c6cc7c8c9ccccc9c9ccccc9c8n(-c8ccccc8)c7cc65)c4nc3-c3ccc4c(c3)C(C)(C)c3ccccc3-4)cc21 ; c1ccc(-c2nc(-c3ccccc3)c3cc(-n4c5ccccc5c5cc6c7c8ccccc8c8ccccc8c7n(-c7ccccc7)c6cc54)ccc3n2)cc1]

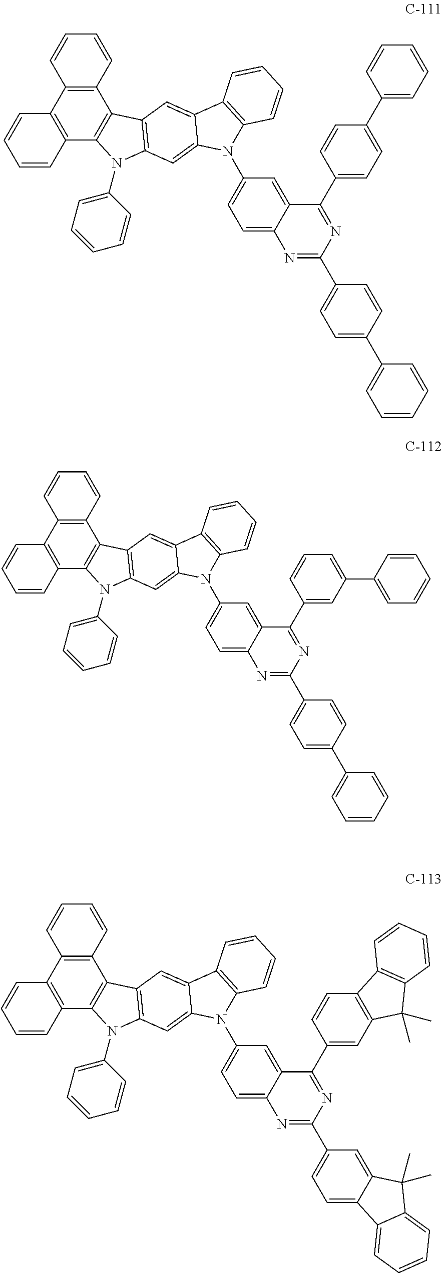 [SMILES: c1ccc(-c2ccc(-c3nc(-c4ccc(-c5ccccc5)cc4)c4cc(-n5c6ccccc6c6cc7c8c9ccccc9c9ccccc9c8n(-c8ccccc8)c7cc65)ccc4n3)cc2)cc1 ; c1ccc(-c2ccc(-c3nc(-c4cccc(-c5ccccc5)c4)c4cc(-n5c6ccccc6c6cc7c8c9ccccc9c9ccccc9c8n(-c8ccccc8)c7cc65)ccc4n3)cc2)cc1 ; CC1(C)c2ccccc2-c2ccc(-c3nc(-c4ccc5c(c4)C(C)(C)c4ccccc4-5)c4cc(-n5c6ccccc6c6cc7c8c9ccccc9c9ccccc9c8n(-c8ccccc8)c7cc65)ccc4n3)cc21]

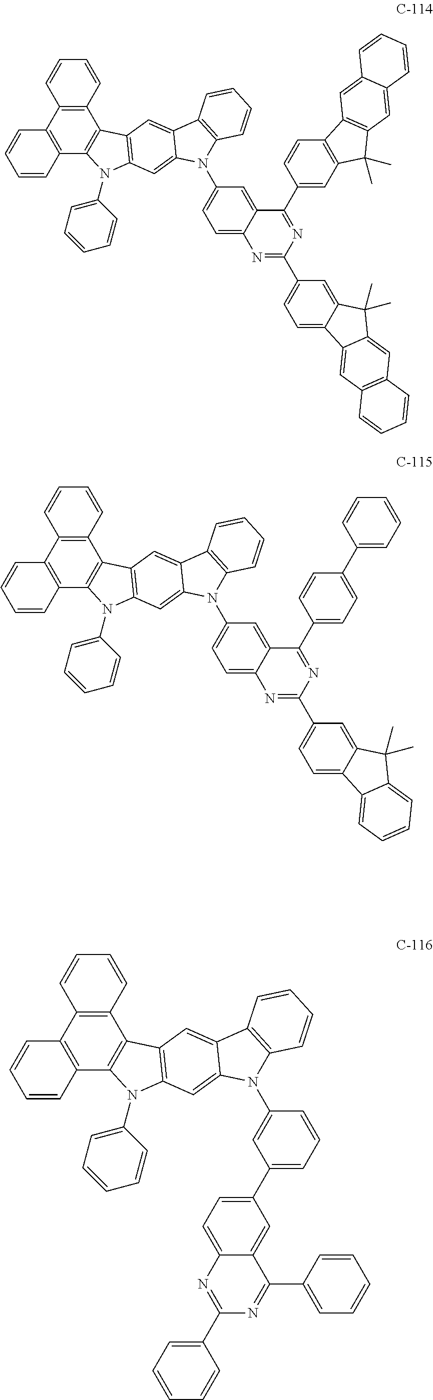 [SMILES: CC1(C)c2cc(-c3nc(-c4ccc5c(c4)C(C)(C)c4cc6ccccc6cc4-5)c4cc(-n5c6ccccc6c6cc7c8c9ccccc9c9ccccc9c8n(-c8ccccc8)c7cc65)ccc4n3)ccc2-c2cc3ccccc3cc21 ; CC1(C)c2ccccc2-c2ccc(-c3nc(-c4ccc(-c5ccccc5)cc4)c4cc(-n5c6ccccc6c6cc7c8c9ccccc9c9ccccc9c8n(-c8ccccc8)c7cc65)ccc4n3)cc21 ; c1ccc(-c2nc(-c3ccccc3)c3cc(-c4cccc(-n5c6ccccc6c6cc7c8c9ccccc9c9ccccc9c8n(-c8ccccc8)c7cc65)c4)ccc3n2)cc1]

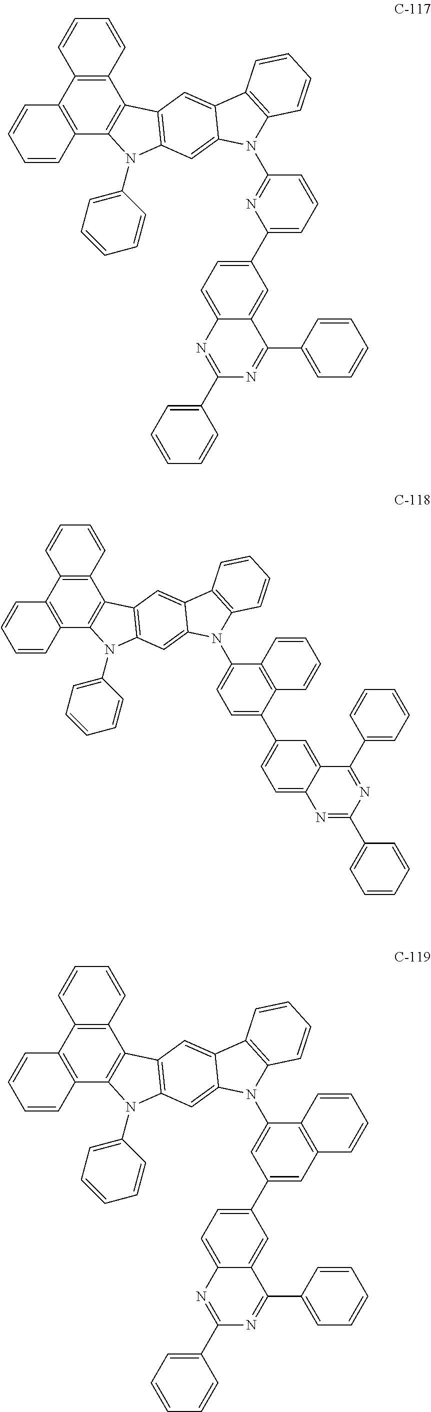 [SMILES: c1ccc(-c2nc(-c3ccccc3)c3cc(-c4cccc(-n5c6ccccc6c6cc7c8c9ccccc9c9ccccc9c8n(-c8ccccc8)c7cc65)n4)ccc3n2)cc1 ; c1ccc(-c2nc(-c3ccccc3)c3cc(-c4ccc(-n5c6ccccc6c6cc7c8c9ccccc9c9ccccc9c8n(-c8ccccc8)c7cc65)c5ccccc45)ccc3n2)cc1 ; c1ccc(-c2nc(-c3ccccc3)c3cc(-c4cc(-n5c6ccccc6c6cc7c8c9ccccc9c9ccccc9c8n(-c8ccccc8)c7cc65)c5ccccc5c4)ccc3n2)cc1]

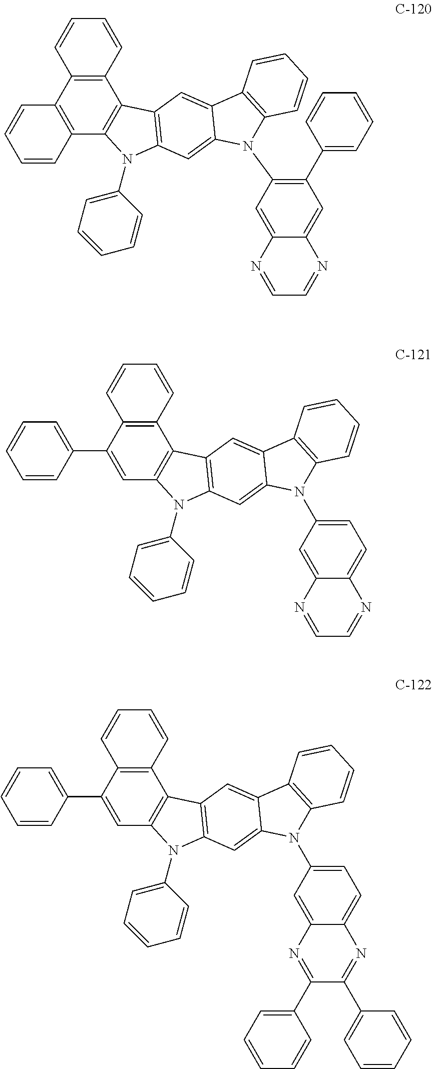 [SMILES: c1ccc(-c2cc3nccnc3cc2-n2c3ccccc3c3cc4c5c6ccccc6c6ccccc6c5n(-c5ccccc5)c4cc32)cc1 ; c1ccc(-c2cc3c(c4ccccc24)c2cc4c5ccccc5n(-c5ccc6nccnc6c5)c4cc2n3-c2ccccc2)cc1 ; c1ccc(-c2nc3ccc(-n4c5ccccc5c5cc6c7c8ccccc8c(-c8ccccc8)cc7n(-c7ccccc7)c6cc54)cc3nc2-c2ccccc2)cc1]

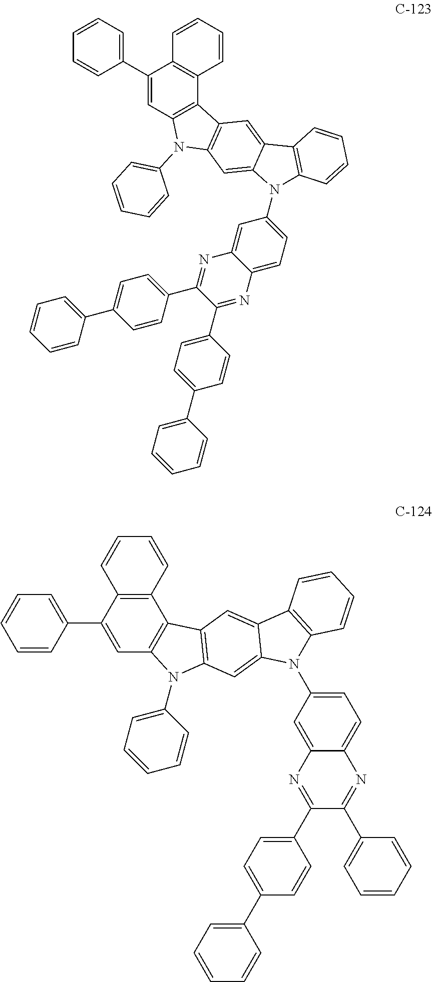 [SMILES: c1ccc(-c2ccc(-c3nc4ccc(-n5c6ccccc6c6cc7c8c9ccccc9c(-c9ccccc9)cc8n(-c8ccccc8)c7cc65)cc4nc3-c3ccc(-c4ccccc4)cc3)cc2)cc1 ; c1ccc(-c2ccc(-c3nc4cc(-n5c6ccccc6c6cc7c8c9ccccc9c(-c9ccccc9)cc8n(-c8ccccc8)c7cc65)ccc4nc3-c3ccccc3)cc2)cc1]

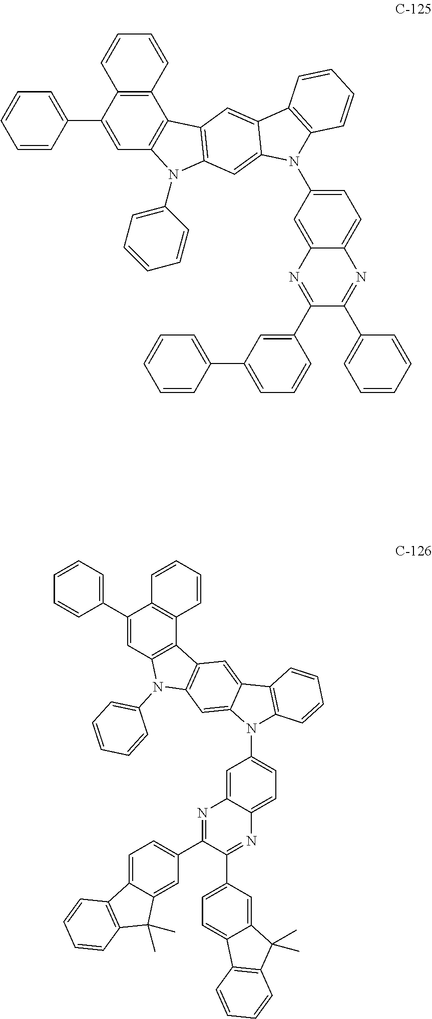 [SMILES: c1ccc(-c2cccc(-c3nc4cc(-n5c6ccccc6c6cc7c8c9ccccc9c(-c9ccccc9)cc8n(-c8ccccc8)c7cc65)ccc4nc3-c3ccccc3)c2)cc1 ; CC1(C)c2ccccc2-c2ccc(-c3nc4ccc(-n5c6ccccc6c6cc7c8c9ccccc9c(-c9ccccc9)cc8n(-c8ccccc8)c7cc65)cc4nc3-c3ccc4c(c3)C(C)(C)c3ccccc3-4)cc21]

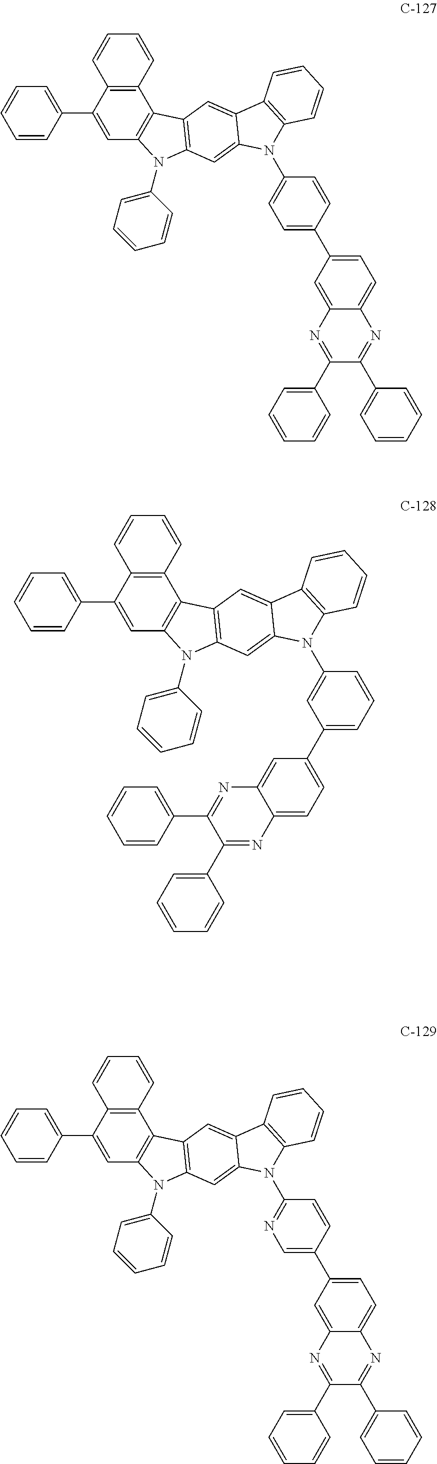 [SMILES: c1ccc(-c2nc3ccc(-c4ccc(-n5c6ccccc6c6cc7c8c9ccccc9c(-c9ccccc9)cc8n(-c8ccccc8)c7cc65)cc4)cc3nc2-c2ccccc2)cc1 ; c1ccc(-c2nc3ccc(-c4cccc(-n5c6ccccc6c6cc7c8c9ccccc9c(-c9ccccc9)cc8n(-c8ccccc8)c7cc65)c4)cc3nc2-c2ccccc2)cc1 ; c1ccc(-c2nc3ccc(-c4ccc(-n5c6ccccc6c6cc7c8c9ccccc9c(-c9ccccc9)cc8n(-c8ccccc8)c7cc65)nc4)cc3nc2-c2ccccc2)cc1]

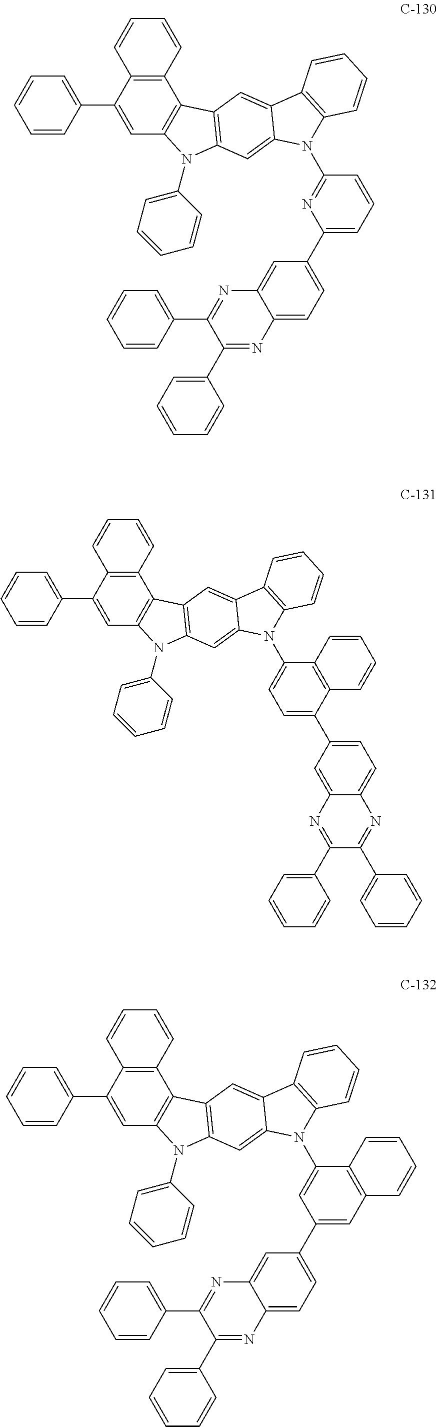 [SMILES: c1ccc(-c2nc3ccc(-c4cccc(-n5c6ccccc6c6cc7c8c9ccccc9c(-c9ccccc9)cc8n(-c8ccccc8)c7cc65)n4)cc3nc2-c2ccccc2)cc1 ; c1ccc(-c2nc3ccc(-c4ccc(-n5c6ccccc6c6cc7c8c9ccccc9c(-c9ccccc9)cc8n(-c8ccccc8)c7cc65)c5ccccc45)cc3nc2-c2ccccc2)cc1 ; c1ccc(-c2nc3ccc(-c4cc(-n5c6ccccc6c6cc7c8c9ccccc9c(-c9ccccc9)cc8n(-c8ccccc8)c7cc65)c5ccccc5c4)cc3nc2-c2ccccc2)cc1]

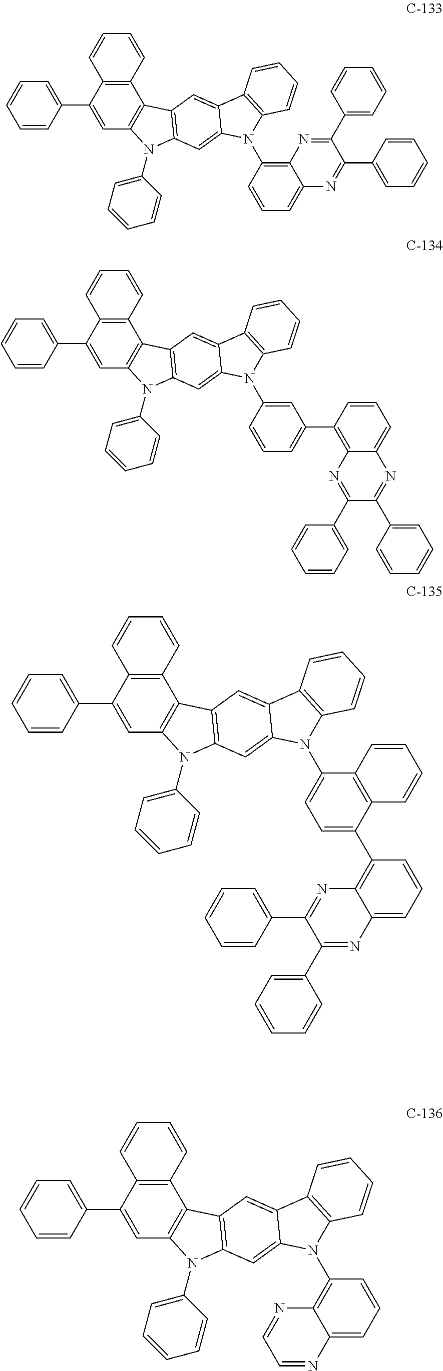 [SMILES: c1ccc(-c2nc3cccc(-n4c5ccccc5c5cc6c7c8ccccc8c(-c8ccccc8)cc7n(-c7ccccc7)c6cc54)c3nc2-c2ccccc2)cc1 ; c1ccc(-c2nc3cccc(-c4cccc(-n5c6ccccc6c6cc7c8c9ccccc9c(-c9ccccc9)cc8n(-c8ccccc8)c7cc65)c4)c3nc2-c2ccccc2)cc1 ; c1ccc(-c2nc3cccc(-c4ccc(-n5c6ccccc6c6cc7c8c9ccccc9c(-c9ccccc9)cc8n(-c8ccccc8)c7cc65)c5ccccc45)c3nc2-c2ccccc2)cc1 ; c1ccc(-c2cc3c(c4ccccc24)c2cc4c5ccccc5n(-c5cccc6nccnc56)c4cc2n3-c2ccccc2)cc1]

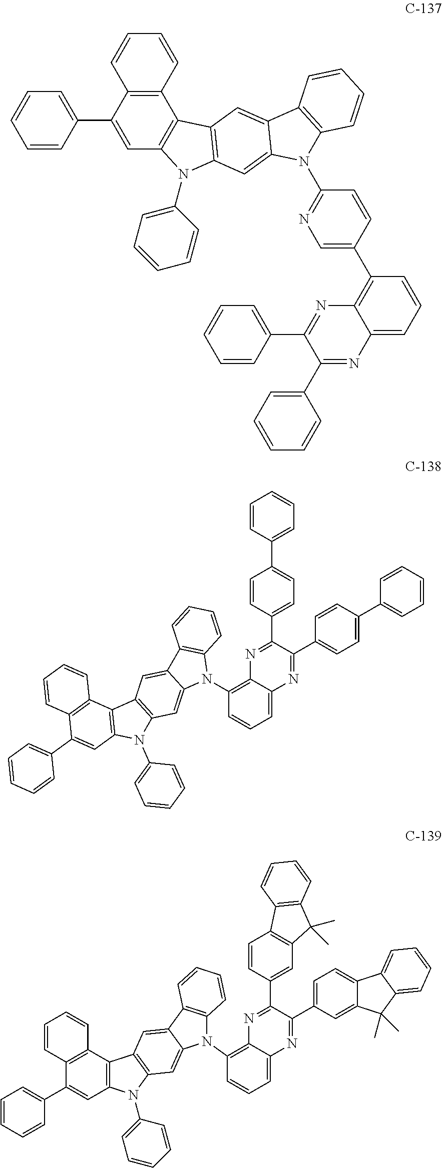 [SMILES: c1ccc(-c2nc3cccc(-c4ccc(-n5c6ccccc6c6cc7c8c9ccccc9c(-c9ccccc9)cc8n(-c8ccccc8)c7cc65)nc4)c3nc2-c2ccccc2)cc1 ; c1ccc(-c2ccc(-c3nc4cccc(-n5c6ccccc6c6cc7c8c9ccccc9c(-c9ccccc9)cc8n(-c8ccccc8)c7cc65)c4nc3-c3ccc(-c4ccccc4)cc3)cc2)cc1 ; CC1(C)c2ccccc2-c2ccc(-c3nc4cccc(-n5c6ccccc6c6cc7c8c9ccccc9c(-c9ccccc9)cc8n(-c8ccccc8)c7cc65)c4nc3-c3ccc4c(c3)C(C)(C)c3ccccc3-4)cc21]

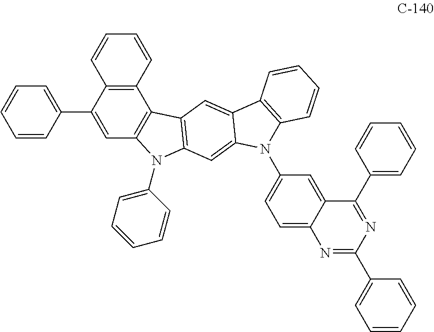 [SMILES: c1ccc(-c2nc(-c3ccccc3)c3cc(-n4c5ccccc5c5cc6c7c8ccccc8c(-c8ccccc8)cc7n(-c7ccccc7)c6cc54)ccc3n2)cc1]

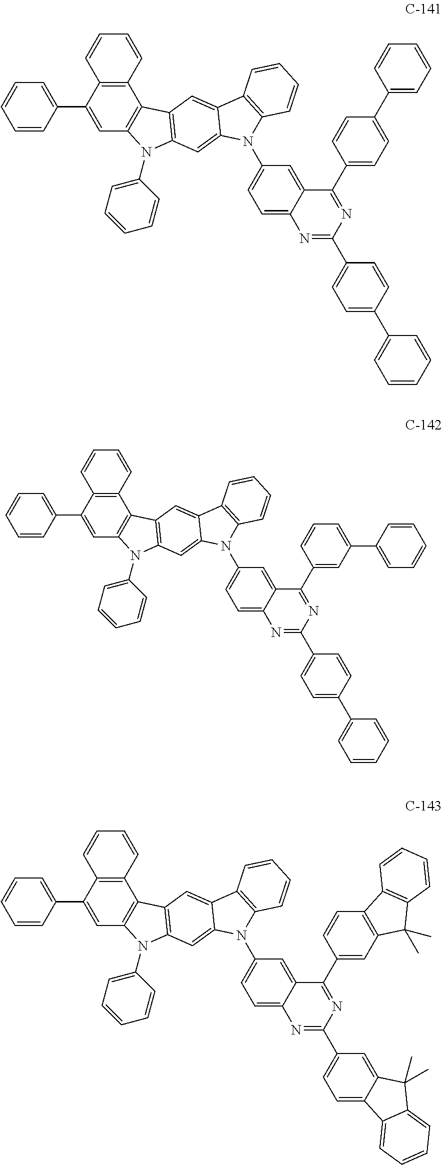 [SMILES: c1ccc(-c2ccc(-c3nc(-c4ccc(-c5ccccc5)cc4)c4cc(-n5c6ccccc6c6cc7c8c9ccccc9c(-c9ccccc9)cc8n(-c8ccccc8)c7cc65)ccc4n3)cc2)cc1 ; c1ccc(-c2ccc(-c3nc(-c4cccc(-c5ccccc5)c4)c4cc(-n5c6ccccc6c6cc7c8c9ccccc9c(-c9ccccc9)cc8n(-c8ccccc8)c7cc65)ccc4n3)cc2)cc1 ; CC1(C)c2ccccc2-c2ccc(-c3nc(-c4ccc5c(c4)C(C)(C)c4ccccc4-5)c4cc(-n5c6ccccc6c6cc7c8c9ccccc9c(-c9ccccc9)cc8n(-c8ccccc8)c7cc65)ccc4n3)cc21]

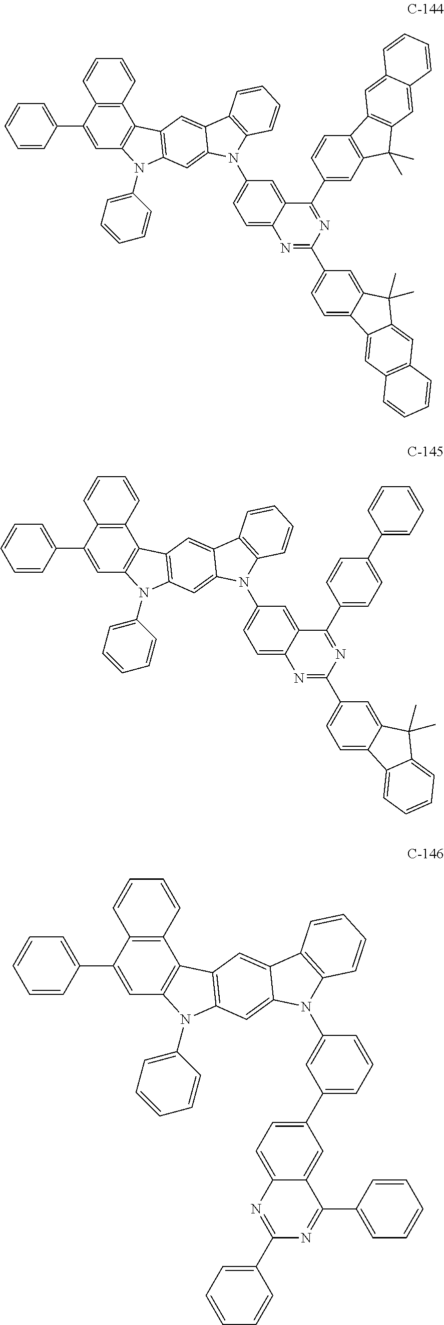 [SMILES: CC1(C)c2cc(-c3nc(-c4ccc5c(c4)C(C)(C)c4cc6ccccc6cc4-5)c4cc(-n5c6ccccc6c6cc7c8c9ccccc9c(-c9ccccc9)cc8n(-c8ccccc8)c7cc65)ccc4n3)ccc2-c2cc3ccccc3cc21 ; CC1(C)c2ccccc2-c2ccc(-c3nc(-c4ccc(-c5ccccc5)cc4)c4cc(-n5c6ccccc6c6cc7c8c9ccccc9c(-c9ccccc9)cc8n(-c8ccccc8)c7cc65)ccc4n3)cc21 ; c1ccc(-c2nc(-c3ccccc3)c3cc(-c4cccc(-n5c6ccccc6c6cc7c8c9ccccc9c(-c9ccccc9)cc8n(-c8ccccc8)c7cc65)c4)ccc3n2)cc1]

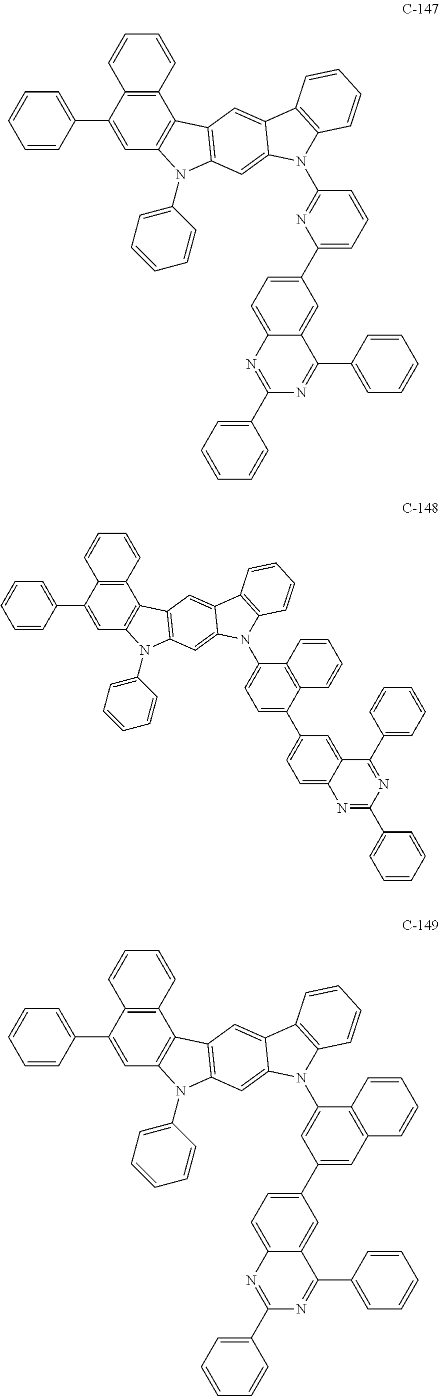 [SMILES: c1ccc(-c2nc(-c3ccccc3)c3cc(-c4cccc(-n5c6ccccc6c6cc7c8c9ccccc9c(-c9ccccc9)cc8n(-c8ccccc8)c7cc65)n4)ccc3n2)cc1 ; c1ccc(-c2nc(-c3ccccc3)c3cc(-c4ccc(-n5c6ccccc6c6cc7c8c9ccccc9c(-c9ccccc9)cc8n(-c8ccccc8)c7cc65)c5ccccc45)ccc3n2)cc1 ; c1ccc(-c2nc(-c3ccccc3)c3cc(-c4cc(-n5c6ccccc6c6cc7c8c9ccccc9c(-c9ccccc9)cc8n(-c8ccccc8)c7cc65)c5ccccc5c4)ccc3n2)cc1]

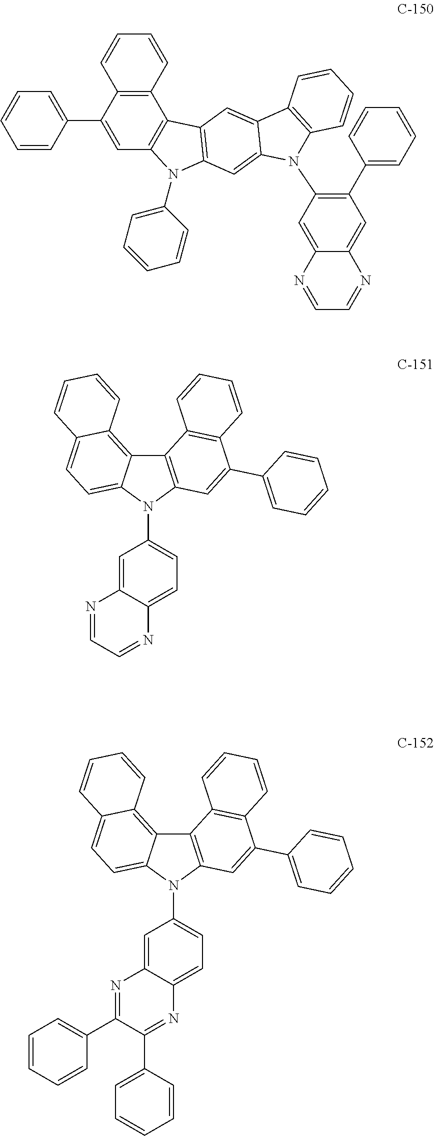 [SMILES: c1ccc(-c2cc3nccnc3cc2-n2c3ccccc3c3cc4c5c6ccccc6c(-c6ccccc6)cc5n(-c5ccccc5)c4cc32)cc1 ; c1ccc(-c2cc3c(c4ccccc24)c2c4ccccc4ccc2n3-c2ccc3nccnc3c2)cc1 ; c1ccc(-c2nc3ccc(-n4c5ccc6ccccc6c5c5c6ccccc6c(-c6ccccc6)cc54)cc3nc2-c2ccccc2)cc1]

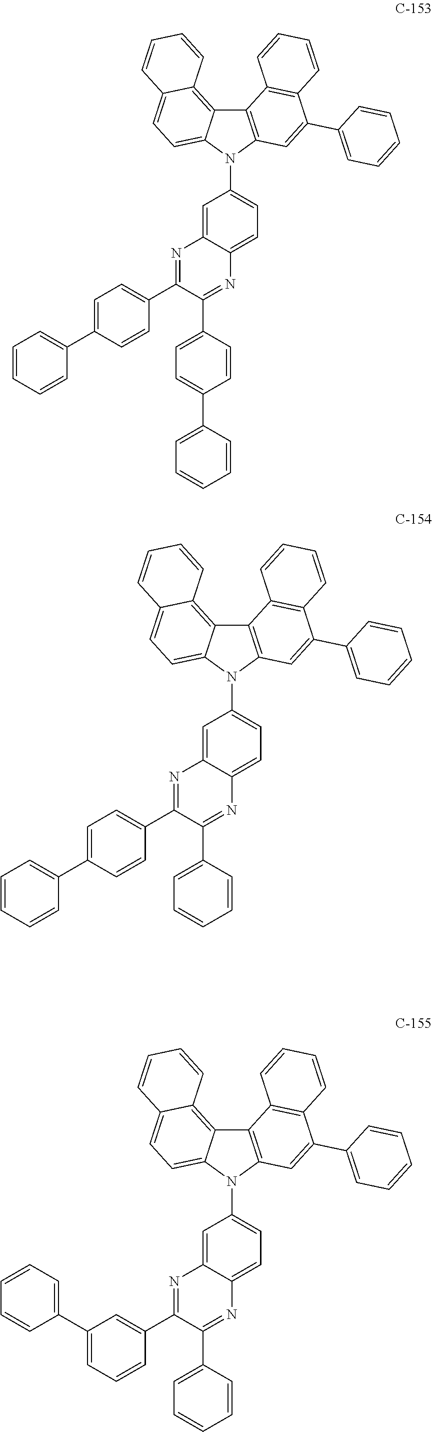 [SMILES: c1ccc(-c2ccc(-c3nc4ccc(-n5c6ccc7ccccc7c6c6c7ccccc7c(-c7ccccc7)cc65)cc4nc3-c3ccc(-c4ccccc4)cc3)cc2)cc1 ; c1ccc(-c2ccc(-c3nc4cc(-n5c6ccc7ccccc7c6c6c7ccccc7c(-c7ccccc7)cc65)ccc4nc3-c3ccccc3)cc2)cc1 ; c1ccc(-c2cccc(-c3nc4cc(-n5c6ccc7ccccc7c6c6c7ccccc7c(-c7ccccc7)cc65)ccc4nc3-c3ccccc3)c2)cc1]

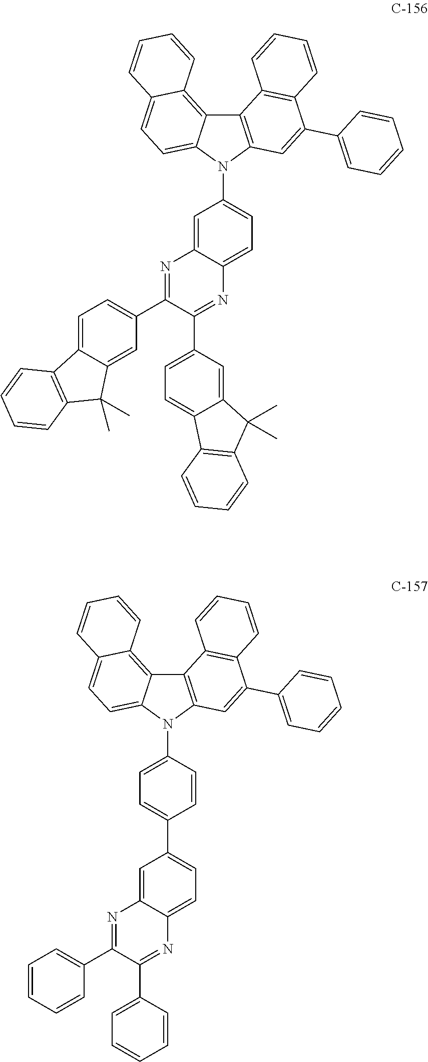 [SMILES: CC1(C)c2ccccc2-c2ccc(-c3nc4ccc(-n5c6ccc7ccccc7c6c6c7ccccc7c(-c7ccccc7)cc65)cc4nc3-c3ccc4c(c3)C(C)(C)c3ccccc3-4)cc21 ; c1ccc(-c2nc3ccc(-c4ccc(-n5c6ccc7ccccc7c6c6c7ccccc7c(-c7ccccc7)cc65)cc4)cc3nc2-c2ccccc2)cc1]

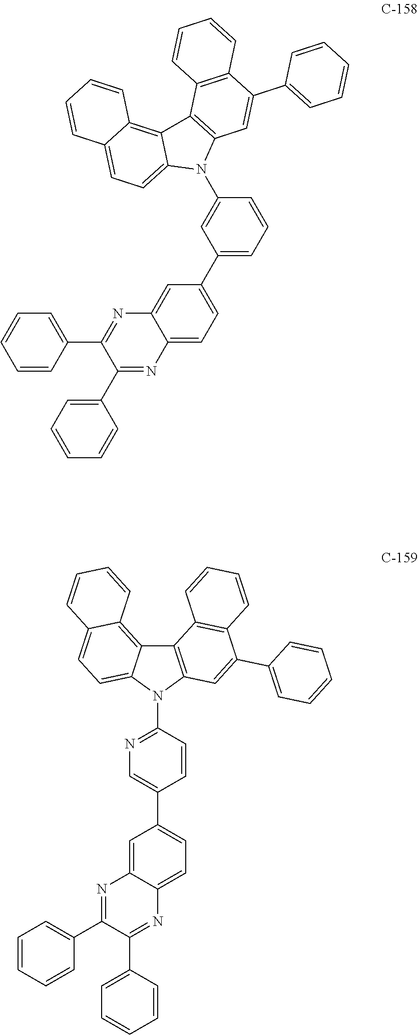 [SMILES: c1ccc(-c2nc3ccc(-c4cccc(-n5c6ccc7ccccc7c6c6c7ccccc7c(-c7ccccc7)cc65)c4)cc3nc2-c2ccccc2)cc1 ; c1ccc(-c2nc3ccc(-c4ccc(-n5c6ccc7ccccc7c6c6c7ccccc7c(-c7ccccc7)cc65)nc4)cc3nc2-c2ccccc2)cc1]

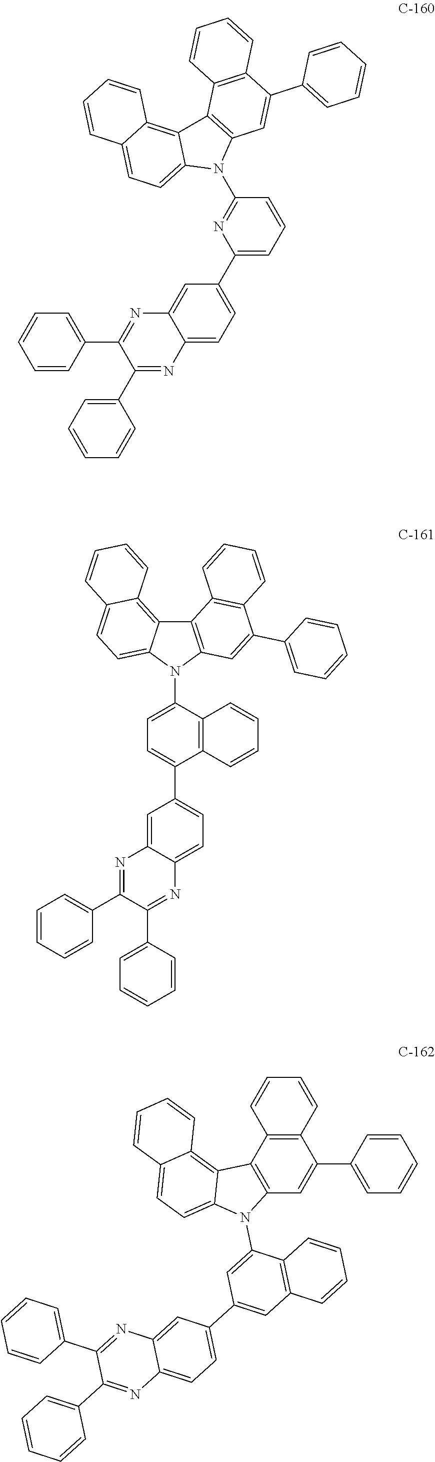 [SMILES: c1ccc(-c2nc3ccc(-c4cccc(-n5c6ccc7ccccc7c6c6c7ccccc7c(-c7ccccc7)cc65)n4)cc3nc2-c2ccccc2)cc1 ; c1ccc(-c2nc3ccc(-c4ccc(-n5c6ccc7ccccc7c6c6c7ccccc7c(-c7ccccc7)cc65)c5ccccc45)cc3nc2-c2ccccc2)cc1 ; c1ccc(-c2nc3ccc(-c4cc(-n5c6ccc7ccccc7c6c6c7ccccc7c(-c7ccccc7)cc65)c5ccccc5c4)cc3nc2-c2ccccc2)cc1]

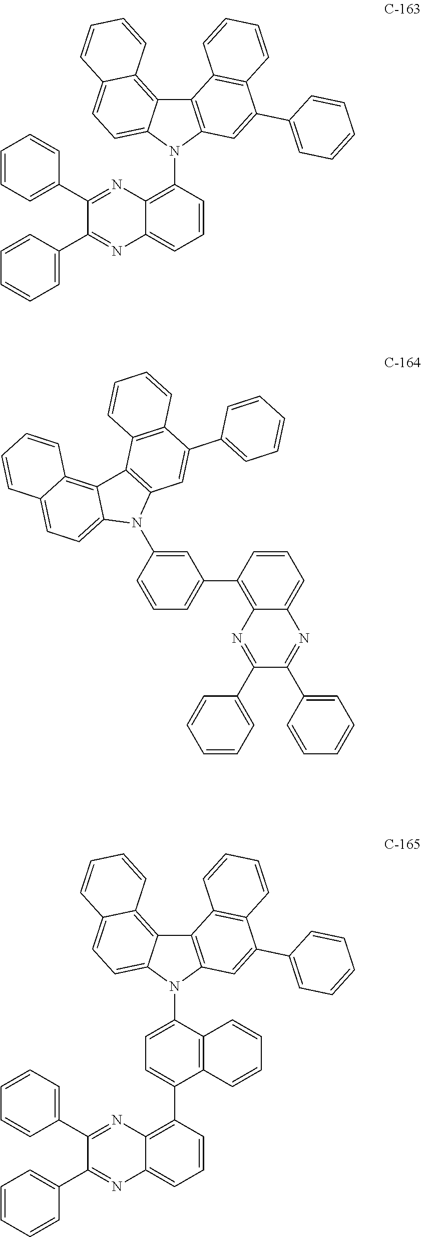 [SMILES: c1ccc(-c2nc3cccc(-n4c5ccc6ccccc6c5c5c6ccccc6c(-c6ccccc6)cc54)c3nc2-c2ccccc2)cc1 ; c1ccc(-c2nc3cccc(-c4cccc(-n5c6ccc7ccccc7c6c6c7ccccc7c(-c7ccccc7)cc65)c4)c3nc2-c2ccccc2)cc1 ; c1ccc(-c2nc3cccc(-c4ccc(-n5c6ccc7ccccc7c6c6c7ccccc7c(-c7ccccc7)cc65)c5ccccc45)c3nc2-c2ccccc2)cc1]

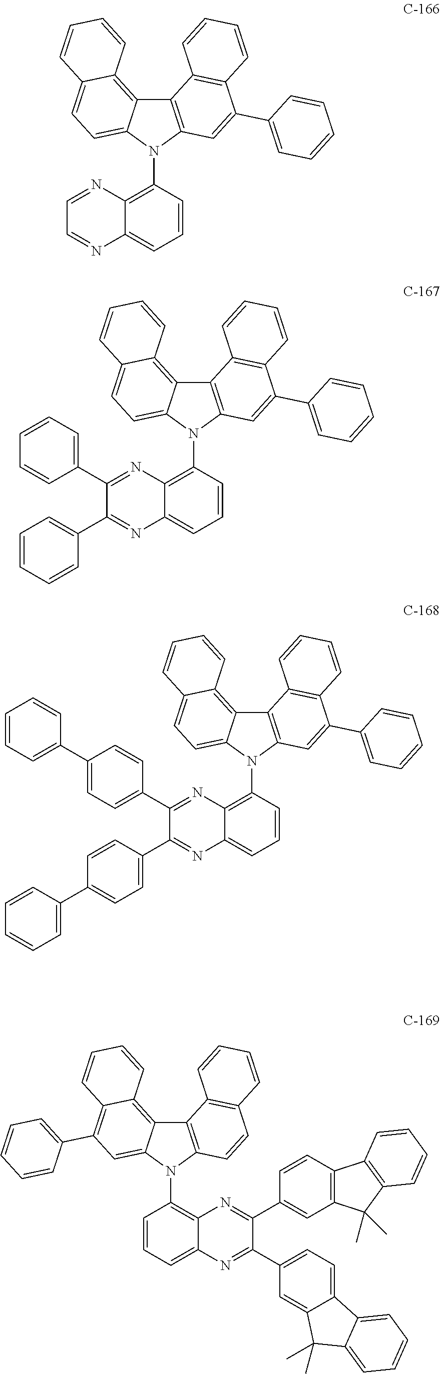 [SMILES: c1ccc(-c2cc3c(c4ccccc24)c2c4ccccc4ccc2n3-c2cccc3nccnc23)cc1 ; c1ccc(-c2nc3cccc(-n4c5ccc6ccccc6c5c5c6ccccc6c(-c6ccccc6)cc54)c3nc2-c2ccccc2)cc1 ; c1ccc(-c2ccc(-c3nc4cccc(-n5c6ccc7ccccc7c6c6c7ccccc7c(-c7ccccc7)cc65)c4nc3-c3ccc(-c4ccccc4)cc3)cc2)cc1 ; CC1(C)c2ccccc2-c2ccc(-c3nc4cccc(-n5c6ccc7ccccc7c6c6c7ccccc7c(-c7ccccc7)cc65)c4nc3-c3ccc4c(c3)C(C)(C)c3ccccc3-4)cc21]

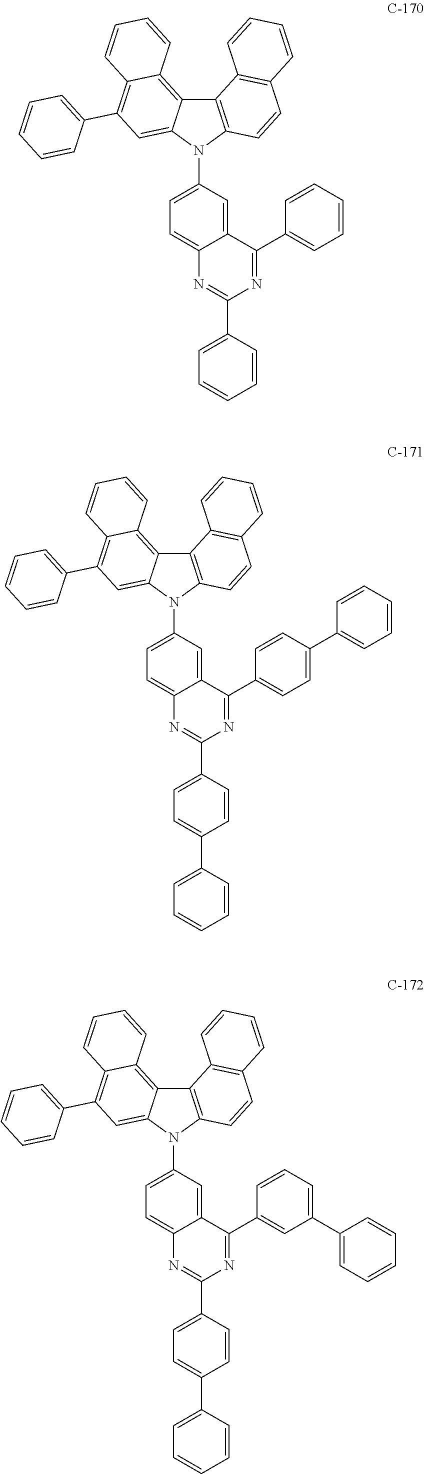 [SMILES: c1ccc(-c2nc(-c3ccccc3)c3cc(-n4c5ccc6ccccc6c5c5c6ccccc6c(-c6ccccc6)cc54)ccc3n2)cc1 ; c1ccc(-c2ccc(-c3nc(-c4ccc(-c5ccccc5)cc4)c4cc(-n5c6ccc7ccccc7c6c6c7ccccc7c(-c7ccccc7)cc65)ccc4n3)cc2)cc1 ; c1ccc(-c2ccc(-c3nc(-c4cccc(-c5ccccc5)c4)c4cc(-n5c6ccc7ccccc7c6c6c7ccccc7c(-c7ccccc7)cc65)ccc4n3)cc2)cc1]

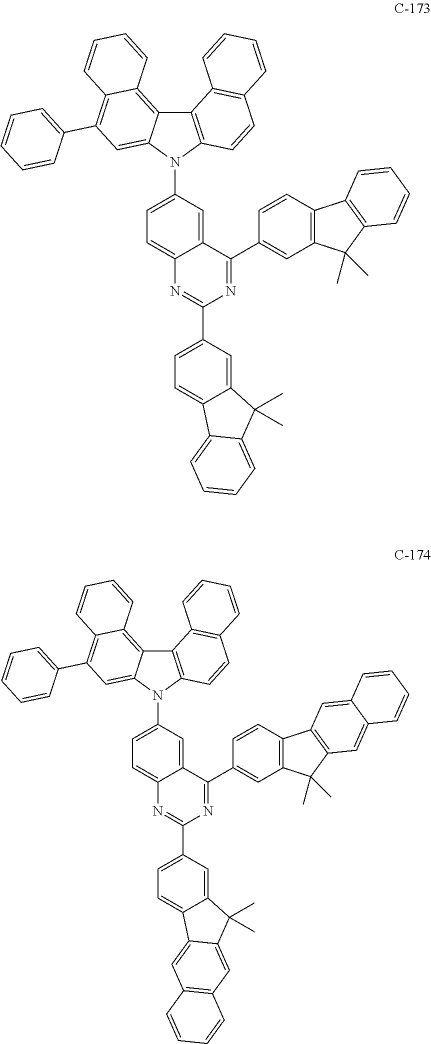 [SMILES: CC1(C)c2ccccc2-c2ccc(-c3nc(-c4ccc5c(c4)C(C)(C)c4ccccc4-5)c4cc(-n5c6ccc7ccccc7c6c6c7ccccc7c(-c7ccccc7)cc65)ccc4n3)cc21 ; CC1(C)c2cc(-c3nc(-c4ccc5c(c4)C(C)(C)c4cc6ccccc6cc4-5)c4cc(-n5c6ccc7ccccc7c6c6c7ccccc7c(-c7ccccc7)cc65)ccc4n3)ccc2-c2cc3ccccc3cc21]

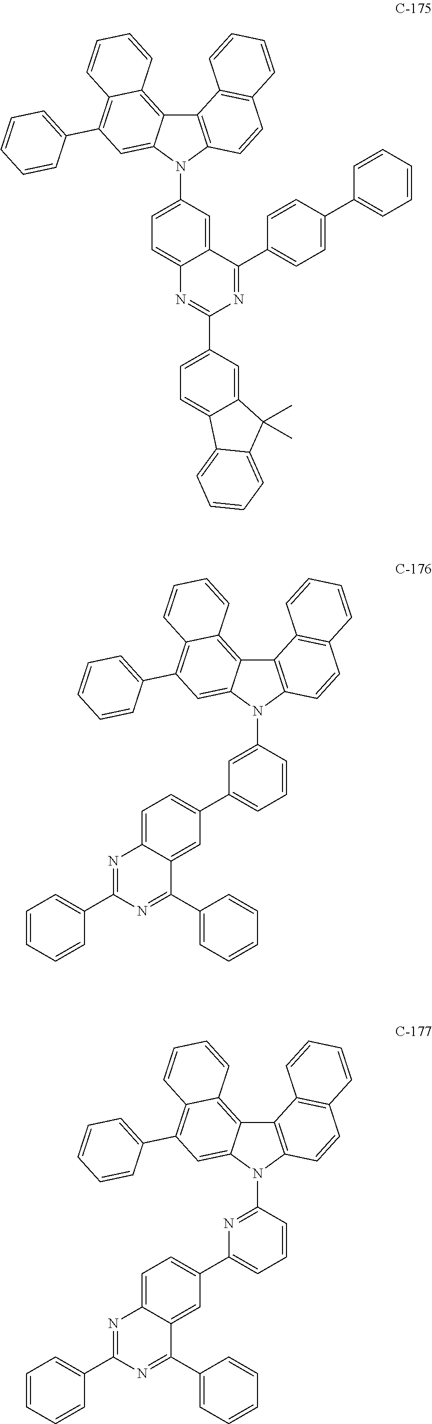 [SMILES: CC1(C)c2ccccc2-c2ccc(-c3nc(-c4ccc(-c5ccccc5)cc4)c4cc(-n5c6ccc7ccccc7c6c6c7ccccc7c(-c7ccccc7)cc65)ccc4n3)cc21 ; c1ccc(-c2nc(-c3ccccc3)c3cc(-c4cccc(-n5c6ccc7ccccc7c6c6c7ccccc7c(-c7ccccc7)cc65)c4)ccc3n2)cc1 ; c1ccc(-c2nc(-c3ccccc3)c3cc(-c4cccc(-n5c6ccc7ccccc7c6c6c7ccccc7c(-c7ccccc7)cc65)n4)ccc3n2)cc1]

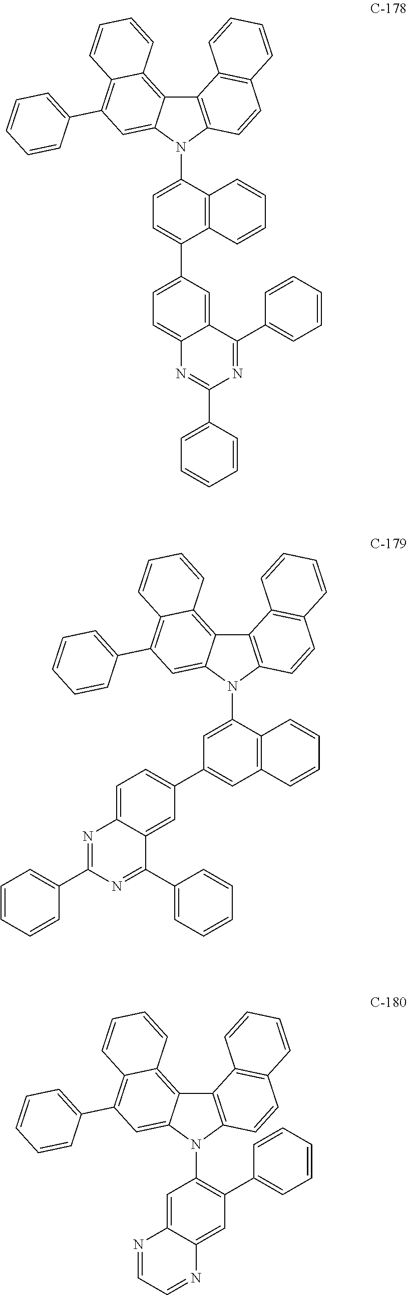 [SMILES: c1ccc(-c2nc(-c3ccccc3)c3cc(-c4ccc(-n5c6ccc7ccccc7c6c6c7ccccc7c(-c7ccccc7)cc65)c5ccccc45)ccc3n2)cc1 ; c1ccc(-c2nc(-c3ccccc3)c3cc(-c4cc(-n5c6ccc7ccccc7c6c6c7ccccc7c(-c7ccccc7)cc65)c5ccccc5c4)ccc3n2)cc1 ; c1ccc(-c2cc3nccnc3cc2-n2c3ccc4ccccc4c3c3c4ccccc4c(-c4ccccc4)cc32)cc1]

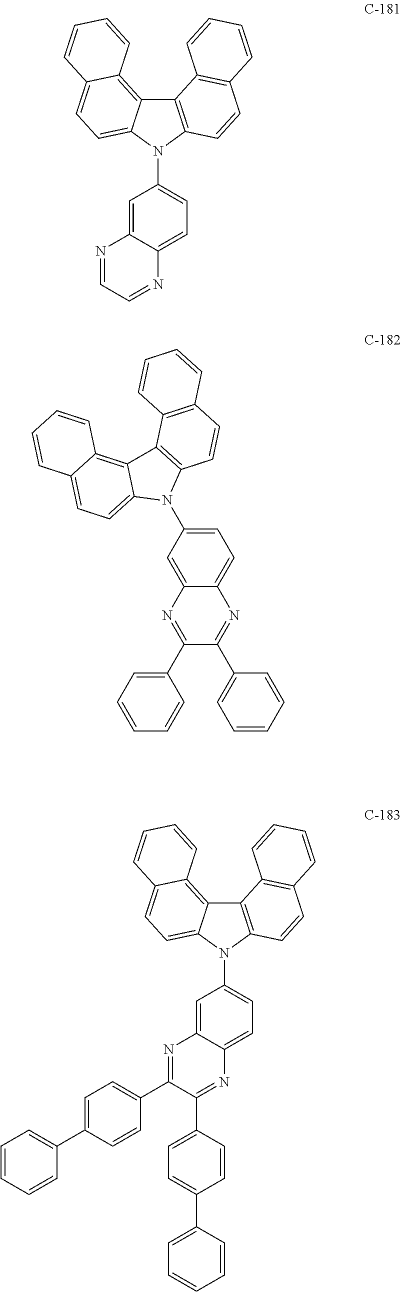 [SMILES: c1ccc2c(c1)ccc1c2c2c3ccccc3ccc2n1-c1ccc2nccnc2c1 ; c1ccc(-c2nc3ccc(-n4c5ccc6ccccc6c5c5c6ccccc6ccc54)cc3nc2-c2ccccc2)cc1 ; c1ccc(-c2ccc(-c3nc4ccc(-n5c6ccc7ccccc7c6c6c7ccccc7ccc65)cc4nc3-c3ccc(-c4ccccc4)cc3)cc2)cc1]

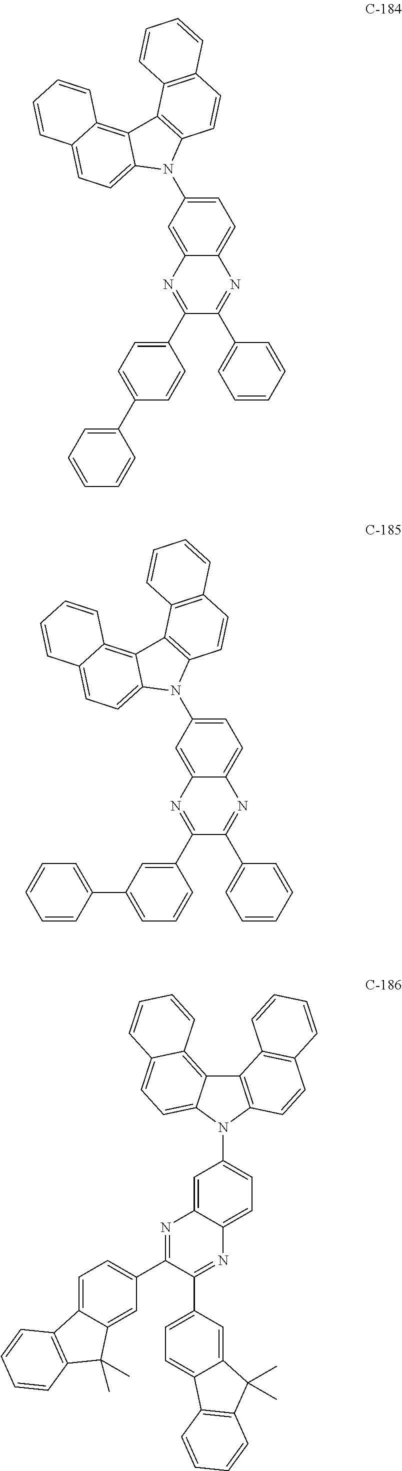 [SMILES: c1ccc(-c2ccc(-c3nc4cc(-n5c6ccc7ccccc7c6c6c7ccccc7ccc65)ccc4nc3-c3ccccc3)cc2)cc1 ; c1ccc(-c2cccc(-c3nc4cc(-n5c6ccc7ccccc7c6c6c7ccccc7ccc65)ccc4nc3-c3ccccc3)c2)cc1 ; CC1(C)c2ccccc2-c2ccc(-c3nc4ccc(-n5c6ccc7ccccc7c6c6c7ccccc7ccc65)cc4nc3-c3ccc4c(c3)C(C)(C)c3ccccc3-4)cc21]

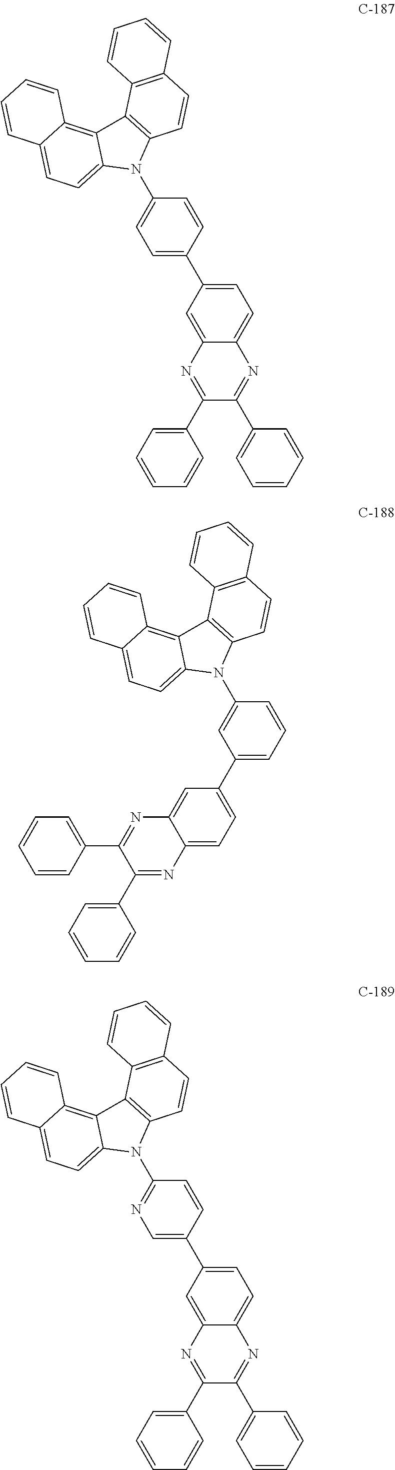

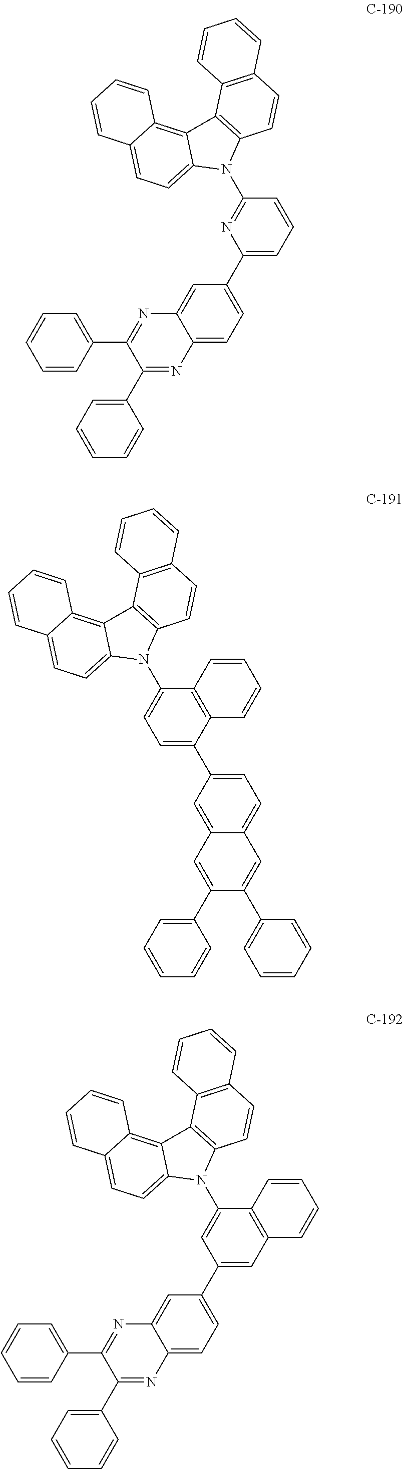 [SMILES: c1ccc(-c2nc3ccc(-c4cccc(-n5c6ccc7ccccc7c6c6c7ccccc7ccc65)n4)cc3nc2-c2ccccc2)cc1 ; c1ccc(-c2cc3ccc(-c4ccc(-n5c6ccc7ccccc7c6c6c7ccccc7ccc65)c5ccccc45)cc3cc2-c2ccccc2)cc1 ; c1ccc(-c2nc3ccc(-c4cc(-n5c6ccc7ccccc7c6c6c7ccccc7ccc65)c5ccccc5c4)cc3nc2-c2ccccc2)cc1]

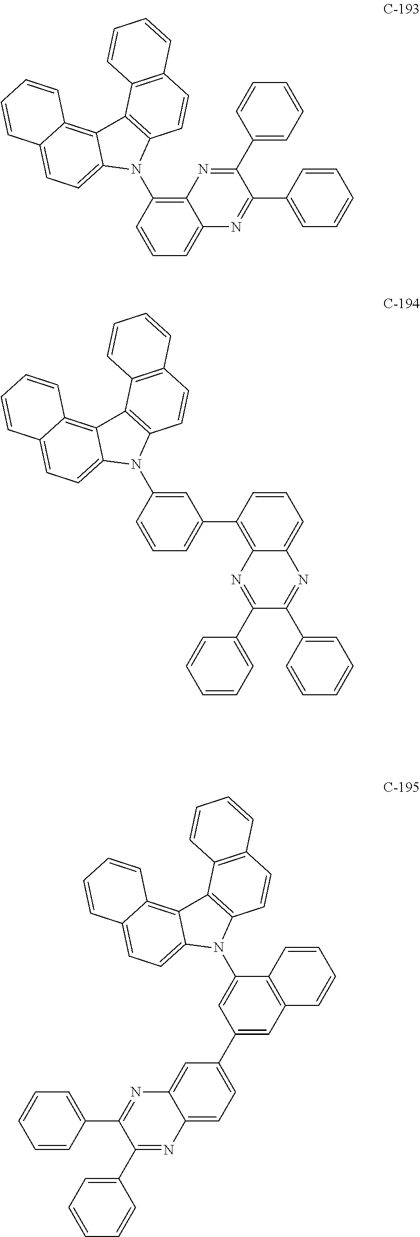 [SMILES: c1ccc(-c2nc3cccc(-n4c5ccc6ccccc6c5c5c6ccccc6ccc54)c3nc2-c2ccccc2)cc1 ; c1ccc(-c2nc3cccc(-c4cccc(-n5c6ccc7ccccc7c6c6c7ccccc7ccc65)c4)c3nc2-c2ccccc2)cc1 ; c1ccc(-c2nc3ccc(-c4cc(-n5c6ccc7ccccc7c6c6c7ccccc7ccc65)c5ccccc5c4)cc3nc2-c2ccccc2)cc1]

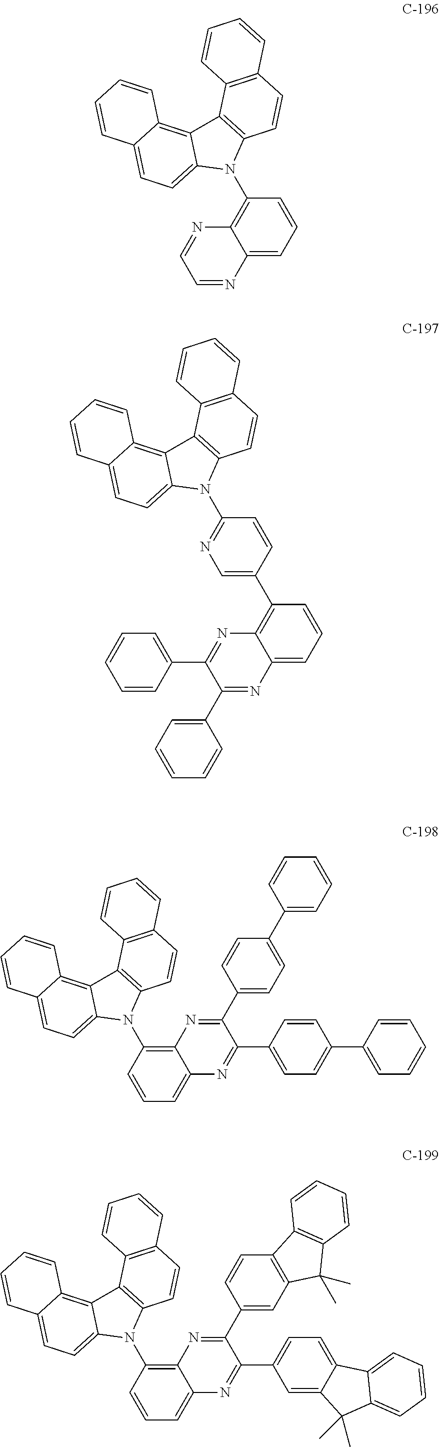 [SMILES: c1ccc2c(c1)ccc1c2c2c3ccccc3ccc2n1-c1cccc2nccnc12 ; c1ccc(-c2nc3cccc(-c4ccc(-n5c6ccc7ccccc7c6c6c7ccccc7ccc65)nc4)c3nc2-c2ccccc2)cc1 ; c1ccc(-c2ccc(-c3nc4cccc(-n5c6ccc7ccccc7c6c6c7ccccc7ccc65)c4nc3-c3ccc(-c4ccccc4)cc3)cc2)cc1 ; CC1(C)c2ccccc2-c2ccc(-c3nc4cccc(-n5c6ccc7ccccc7c6c6c7ccccc7ccc65)c4nc3-c3ccc4c(c3)C(C)(C)c3ccccc3-4)cc21]

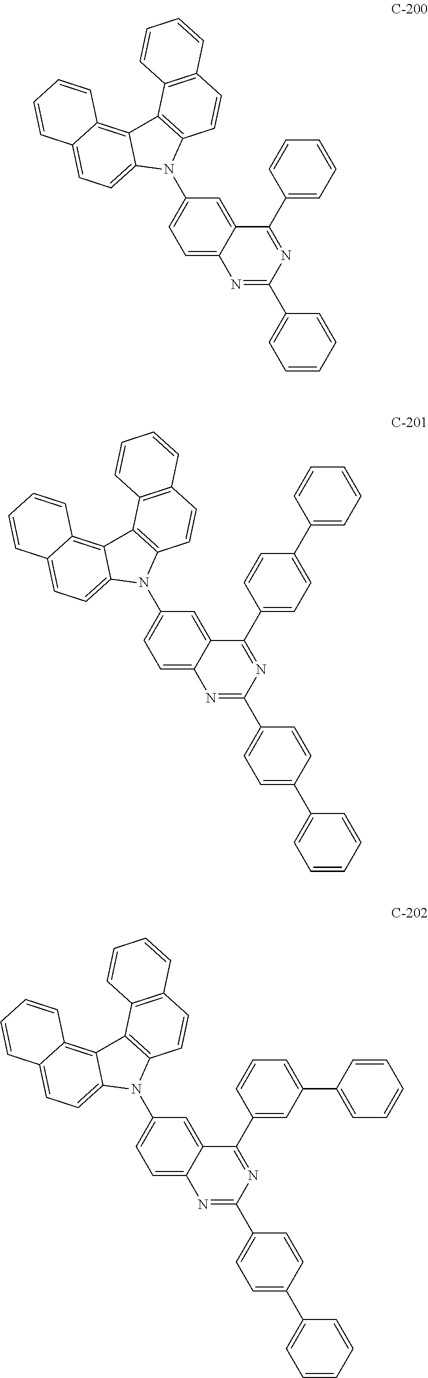 [SMILES: c1ccc(-c2nc(-c3ccccc3)c3cc(-n4c5ccc6ccccc6c5c5c6ccccc6ccc54)ccc3n2)cc1 ; c1ccc(-c2ccc(-c3nc(-c4ccc(-c5ccccc5)cc4)c4cc(-n5c6ccc7ccccc7c6c6c7ccccc7ccc65)ccc4n3)cc2)cc1 ; c1ccc(-c2ccc(-c3nc(-c4cccc(-c5ccccc5)c4)c4cc(-n5c6ccc7ccccc7c6c6c7ccccc7ccc65)ccc4n3)cc2)cc1]

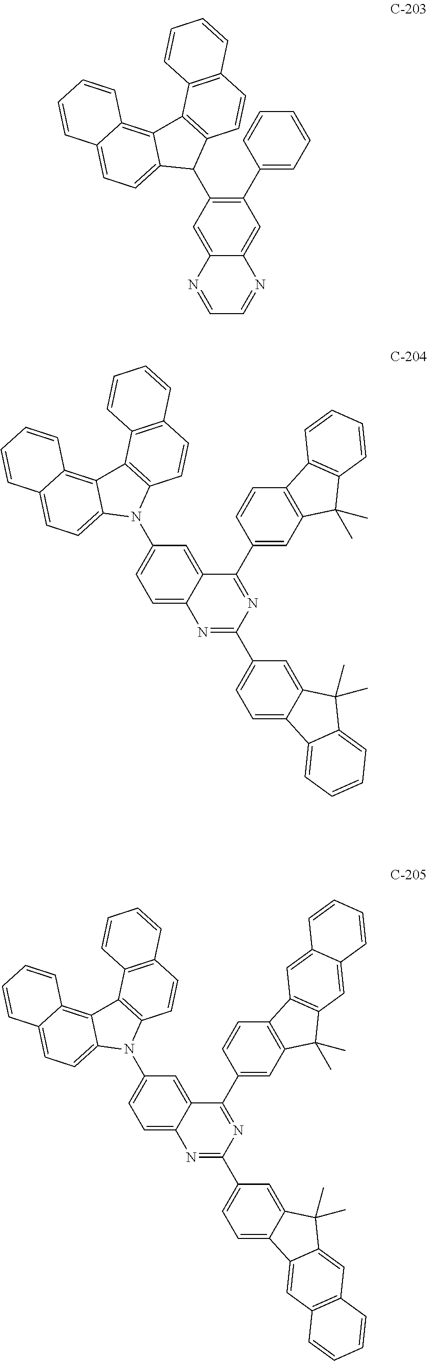 [SMILES: c1ccc(-c2cc3nccnc3cc2C2c3ccc4ccccc4c3-c3c2ccc2ccccc32)cc1 ; CC1(C)c2ccccc2-c2ccc(-c3nc(-c4ccc5c(c4)C(C)(C)c4ccccc4-5)c4cc(-n5c6ccc7ccccc7c6c6c7ccccc7ccc65)ccc4n3)cc21 ; CC1(C)c2cc(-c3nc(-c4ccc5c(c4)C(C)(C)c4cc6ccccc6cc4-5)c4cc(-n5c6ccc7ccccc7c6c6c7ccccc7ccc65)ccc4n3)ccc2-c2cc3ccccc3cc21]

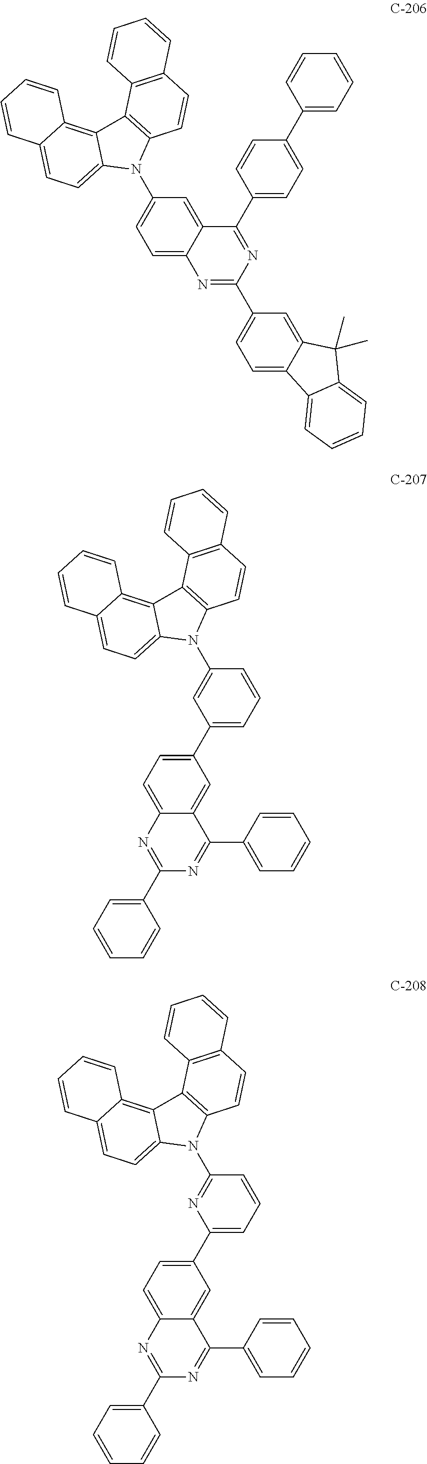 [SMILES: CC1(C)c2ccccc2-c2ccc(-c3nc(-c4ccc(-c5ccccc5)cc4)c4cc(-n5c6ccc7ccccc7c6c6c7ccccc7ccc65)ccc4n3)cc21 ; c1ccc(-c2nc(-c3ccccc3)c3cc(-c4cccc(-n5c6ccc7ccccc7c6c6c7ccccc7ccc65)c4)ccc3n2)cc1 ; c1ccc(-c2nc(-c3ccccc3)c3cc(-c4cccc(-n5c6ccc7ccccc7c6c6c7ccccc7ccc65)n4)ccc3n2)cc1]

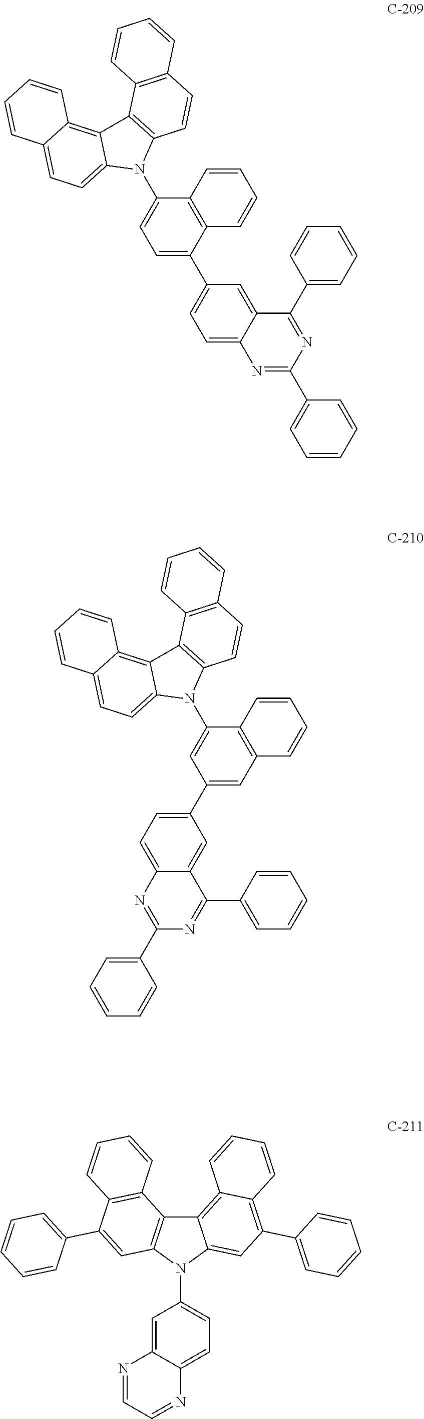 [SMILES: c1ccc(-c2nc(-c3ccccc3)c3cc(-c4ccc(-n5c6ccc7ccccc7c6c6c7ccccc7ccc65)c5ccccc45)ccc3n2)cc1 ; c1ccc(-c2nc(-c3ccccc3)c3cc(-c4cc(-n5c6ccc7ccccc7c6c6c7ccccc7ccc65)c5ccccc5c4)ccc3n2)cc1 ; c1ccc(-c2cc3c(c4ccccc24)c2c4ccccc4c(-c4ccccc4)cc2n3-c2ccc3nccnc3c2)cc1]

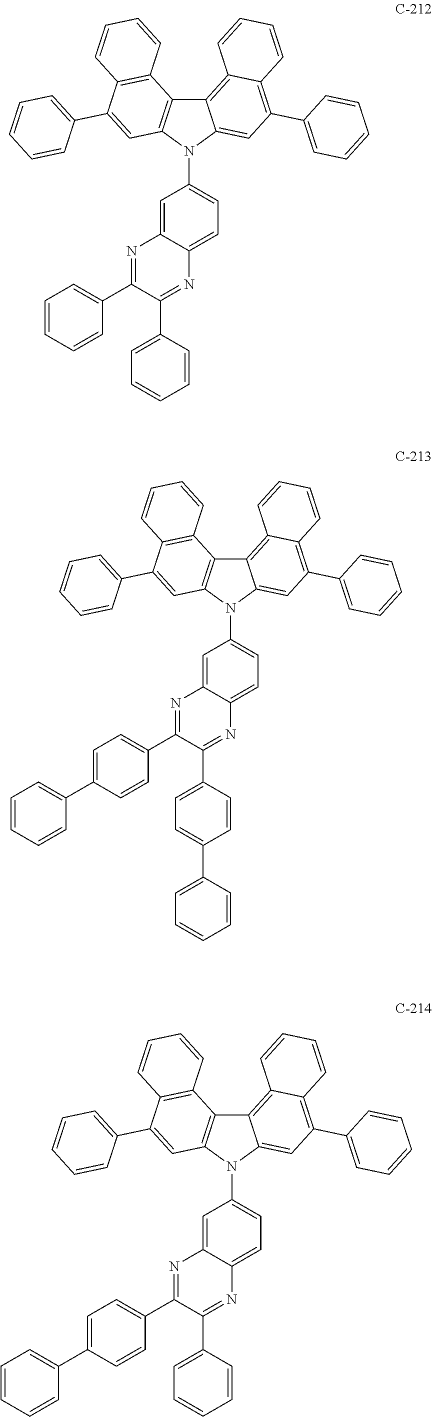 [SMILES: c1ccc(-c2nc3ccc(-n4c5cc(-c6ccccc6)c6ccccc6c5c5c6ccccc6c(-c6ccccc6)cc54)cc3nc2-c2ccccc2)cc1 ; c1ccc(-c2ccc(-c3nc4ccc(-n5c6cc(-c7ccccc7)c7ccccc7c6c6c7ccccc7c(-c7ccccc7)cc65)cc4nc3-c3ccc(-c4ccccc4)cc3)cc2)cc1 ; c1ccc(-c2ccc(-c3nc4cc(-n5c6cc(-c7ccccc7)c7ccccc7c6c6c7ccccc7c(-c7ccccc7)cc65)ccc4nc3-c3ccccc3)cc2)cc1]

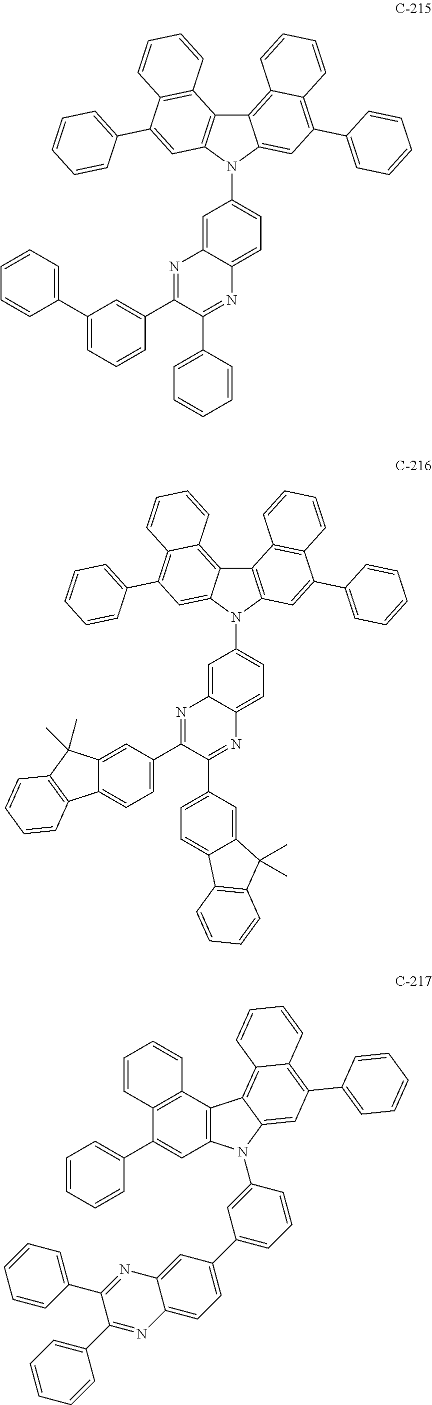 [SMILES: c1ccc(-c2cccc(-c3nc4cc(-n5c6cc(-c7ccccc7)c7ccccc7c6c6c7ccccc7c(-c7ccccc7)cc65)ccc4nc3-c3ccccc3)c2)cc1 ; CC1(C)c2ccccc2-c2ccc(-c3nc4ccc(-n5c6cc(-c7ccccc7)c7ccccc7c6c6c7ccccc7c(-c7ccccc7)cc65)cc4nc3-c3ccc4c(c3)C(C)(C)c3ccccc3-4)cc21 ; c1ccc(-c2nc3ccc(-c4cccc(-n5c6cc(-c7ccccc7)c7ccccc7c6c6c7ccccc7c(-c7ccccc7)cc65)c4)cc3nc2-c2ccccc2)cc1]

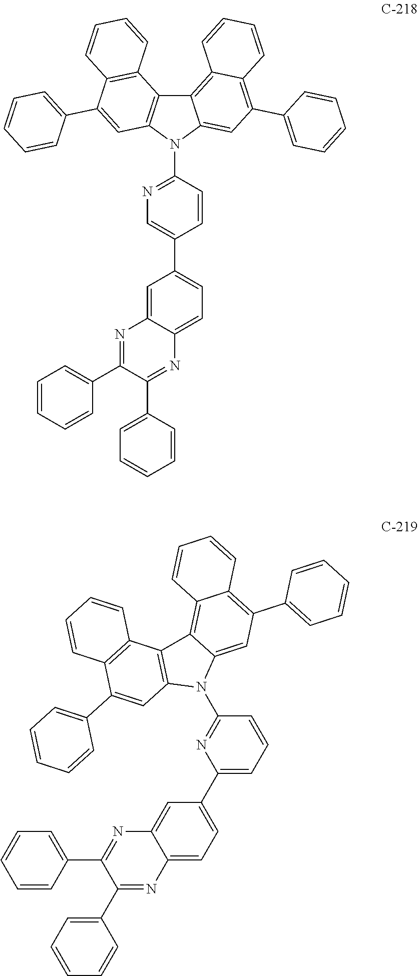 [SMILES: c1ccc(-c2nc3ccc(-c4ccc(-n5c6cc(-c7ccccc7)c7ccccc7c6c6c7ccccc7c(-c7ccccc7)cc65)nc4)cc3nc2-c2ccccc2)cc1 ; c1ccc(-c2nc3ccc(-c4cccc(-n5c6cc(-c7ccccc7)c7ccccc7c6c6c7ccccc7c(-c7ccccc7)cc65)n4)cc3nc2-c2ccccc2)cc1]

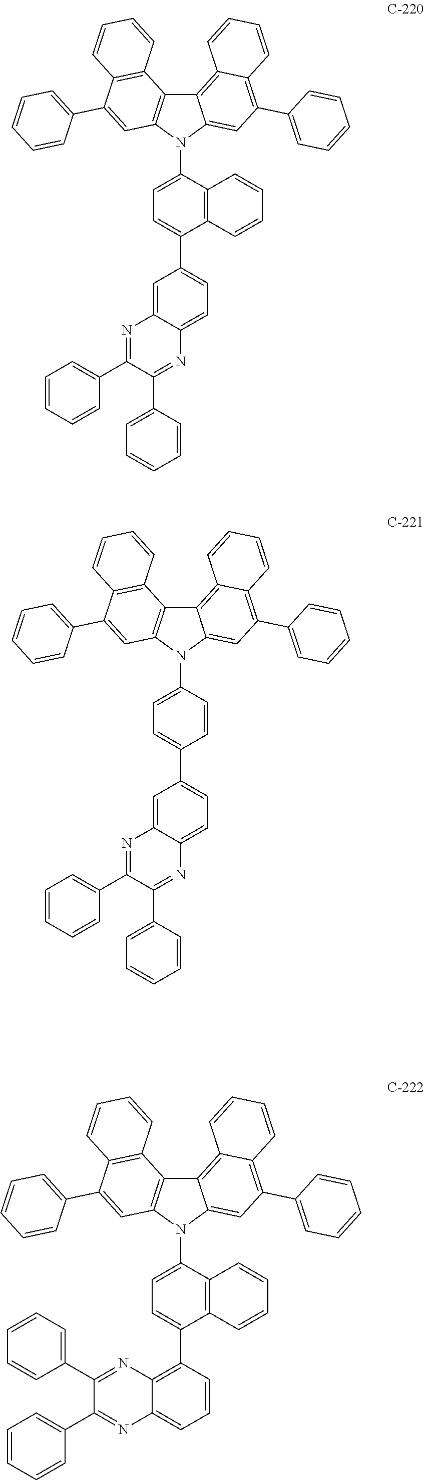 [SMILES: c1ccc(-c2nc3ccc(-c4ccc(-n5c6cc(-c7ccccc7)c7ccccc7c6c6c7ccccc7c(-c7ccccc7)cc65)c5ccccc45)cc3nc2-c2ccccc2)cc1 ; c1ccc(-c2nc3ccc(-c4ccc(-n5c6cc(-c7ccccc7)c7ccccc7c6c6c7ccccc7c(-c7ccccc7)cc65)cc4)cc3nc2-c2ccccc2)cc1 ; c1ccc(-c2nc3cccc(-c4ccc(-n5c6cc(-c7ccccc7)c7ccccc7c6c6c7ccccc7c(-c7ccccc7)cc65)c5ccccc45)c3nc2-c2ccccc2)cc1]

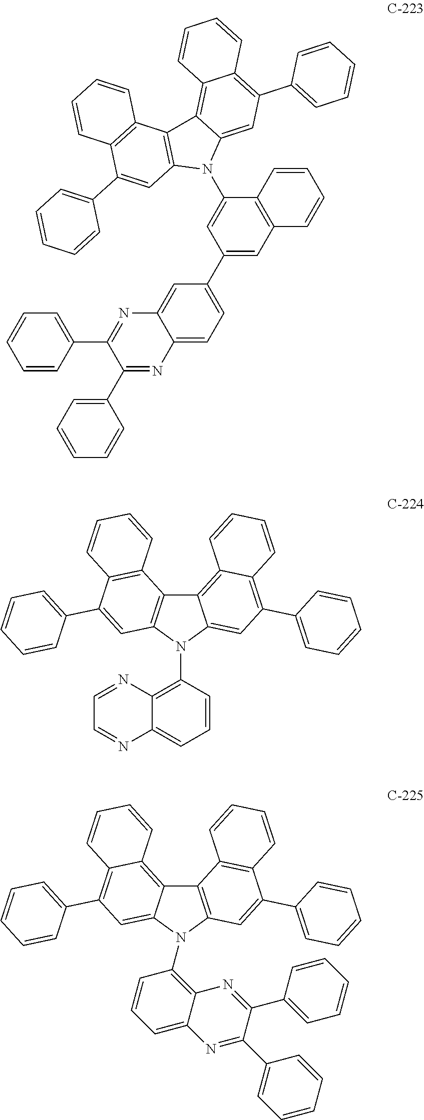 [SMILES: c1ccc(-c2nc3ccc(-c4cc(-n5c6cc(-c7ccccc7)c7ccccc7c6c6c7ccccc7c(-c7ccccc7)cc65)c5ccccc5c4)cc3nc2-c2ccccc2)cc1 ; c1ccc(-c2cc3c(c4ccccc24)c2c4ccccc4c(-c4ccccc4)cc2n3-c2cccc3nccnc23)cc1 ; c1ccc(-c2nc3cccc(-n4c5cc(-c6ccccc6)c6ccccc6c5c5c6ccccc6c(-c6ccccc6)cc54)c3nc2-c2ccccc2)cc1]

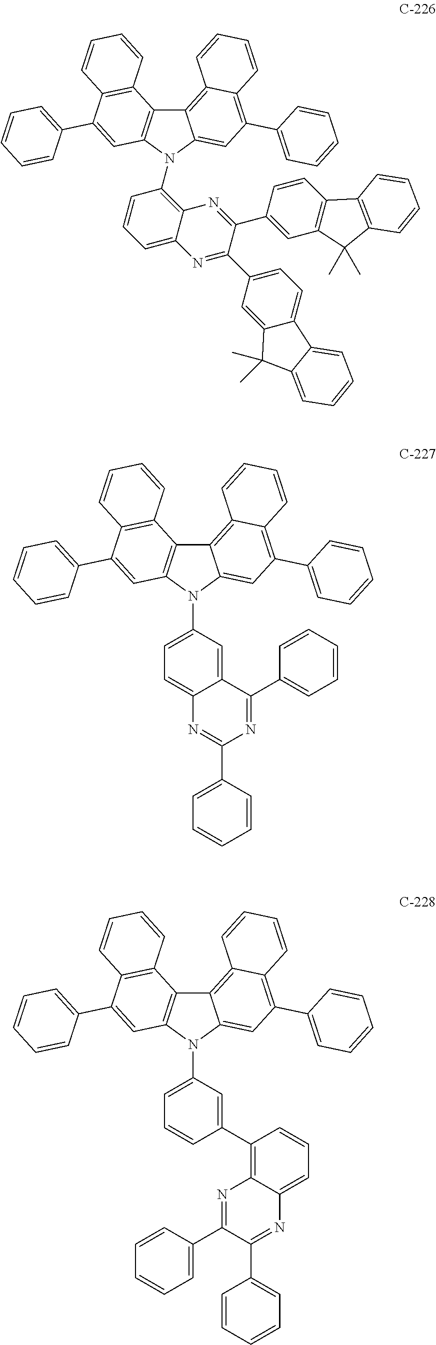 [SMILES: CC1(C)c2ccccc2-c2ccc(-c3nc4cccc(-n5c6cc(-c7ccccc7)c7ccccc7c6c6c7ccccc7c(-c7ccccc7)cc65)c4nc3-c3ccc4c(c3)C(C)(C)c3ccccc3-4)cc21 ; c1ccc(-c2nc(-c3ccccc3)c3cc(-n4c5cc(-c6ccccc6)c6ccccc6c5c5c6ccccc6c(-c6ccccc6)cc54)ccc3n2)cc1 ; c1ccc(-c2nc3cccc(-c4cccc(-n5c6cc(-c7ccccc7)c7ccccc7c6c6c7ccccc7c(-c7ccccc7)cc65)c4)c3nc2-c2ccccc2)cc1]

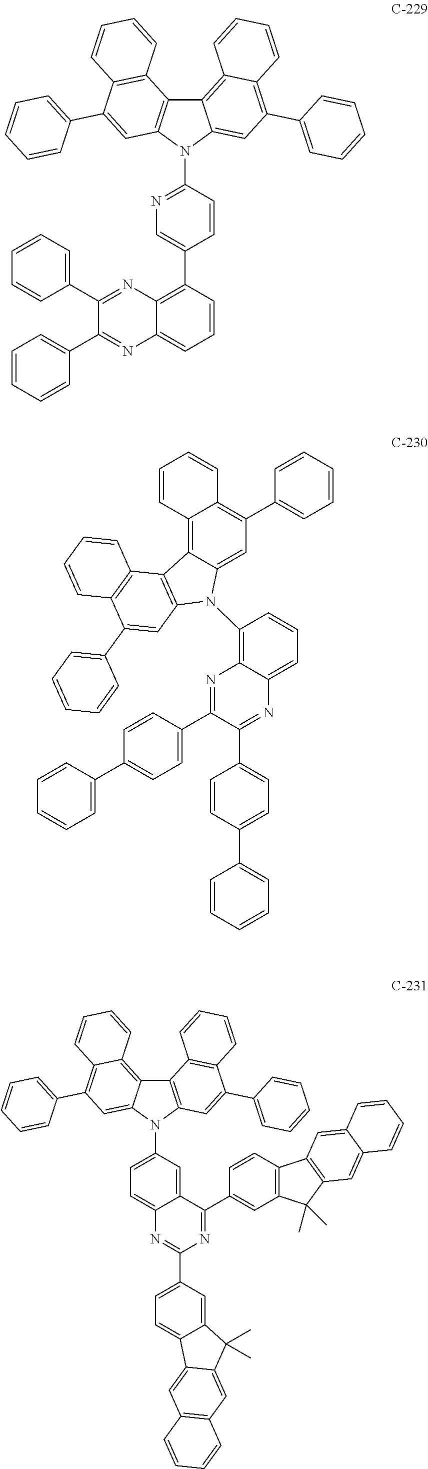 [SMILES: c1ccc(-c2nc3cccc(-c4ccc(-n5c6cc(-c7ccccc7)c7ccccc7c6c6c7ccccc7c(-c7ccccc7)cc65)nc4)c3nc2-c2ccccc2)cc1 ; c1ccc(-c2ccc(-c3nc4cccc(-n5c6cc(-c7ccccc7)c7ccccc7c6c6c7ccccc7c(-c7ccccc7)cc65)c4nc3-c3ccc(-c4ccccc4)cc3)cc2)cc1 ; CC1(C)c2cc(-c3nc(-c4ccc5c(c4)C(C)(C)c4cc6ccccc6cc4-5)c4cc(-n5c6cc(-c7ccccc7)c7ccccc7c6c6c7ccccc7c(-c7ccccc7)cc65)ccc4n3)ccc2-c2cc3ccccc3cc21]

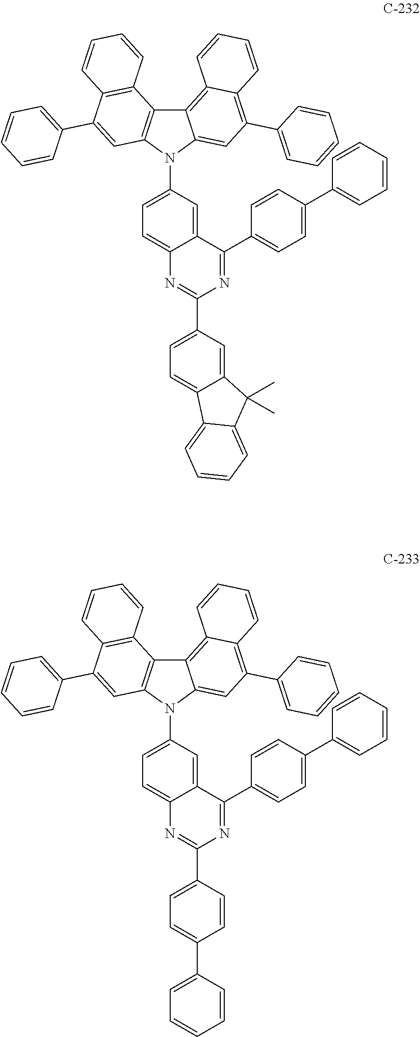 [SMILES: CC1(C)c2ccccc2-c2ccc(-c3nc(-c4ccc(-c5ccccc5)cc4)c4cc(-n5c6cc(-c7ccccc7)c7ccccc7c6c6c7ccccc7c(-c7ccccc7)cc65)ccc4n3)cc21 ; c1ccc(-c2ccc(-c3nc(-c4ccc(-c5ccccc5)cc4)c4cc(-n5c6cc(-c7ccccc7)c7ccccc7c6c6c7ccccc7c(-c7ccccc7)cc65)ccc4n3)cc2)cc1]

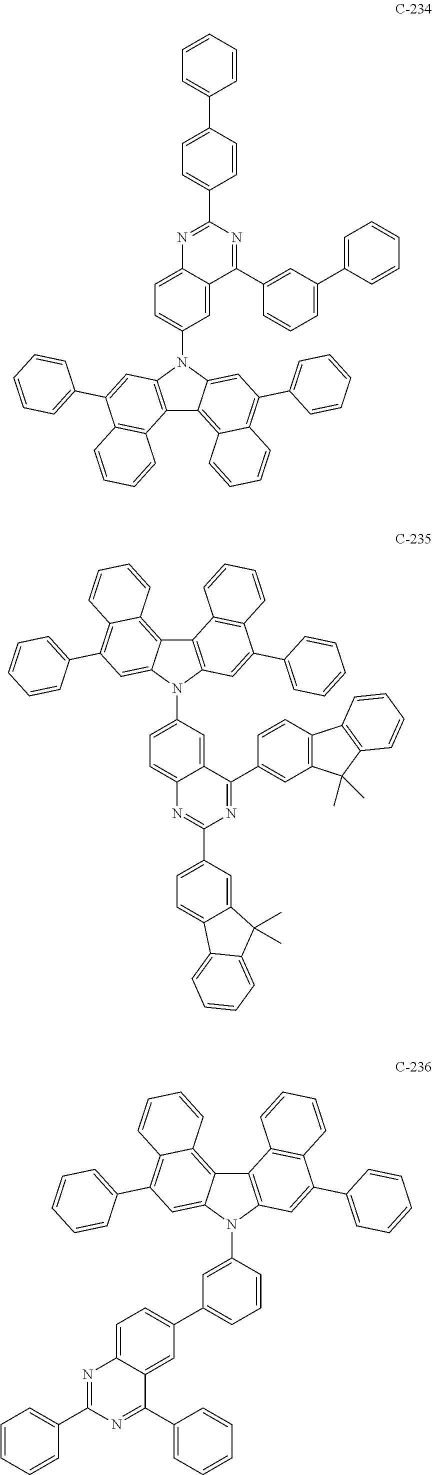 [SMILES: c1ccc(-c2ccc(-c3nc(-c4cccc(-c5ccccc5)c4)c4cc(-n5c6cc(-c7ccccc7)c7ccccc7c6c6c7ccccc7c(-c7ccccc7)cc65)ccc4n3)cc2)cc1 ; CC1(C)c2ccccc2-c2ccc(-c3nc(-c4ccc5c(c4)C(C)(C)c4ccccc4-5)c4cc(-n5c6cc(-c7ccccc7)c7ccccc7c6c6c7ccccc7c(-c7ccccc7)cc65)ccc4n3)cc21 ; c1ccc(-c2nc(-c3ccccc3)c3cc(-c4cccc(-n5c6cc(-c7ccccc7)c7ccccc7c6c6c7ccccc7c(-c7ccccc7)cc65)c4)ccc3n2)cc1]

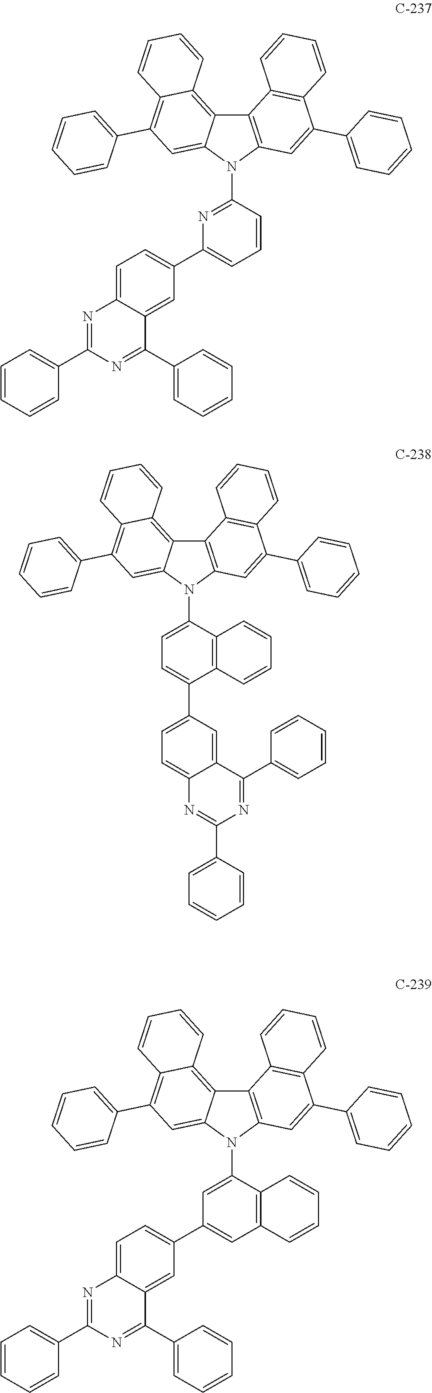 [SMILES: c1ccc(-c2nc(-c3ccccc3)c3cc(-c4cccc(-n5c6cc(-c7ccccc7)c7ccccc7c6c6c7ccccc7c(-c7ccccc7)cc65)n4)ccc3n2)cc1 ; c1ccc(-c2nc(-c3ccccc3)c3cc(-c4ccc(-n5c6cc(-c7ccccc7)c7ccccc7c6c6c7ccccc7c(-c7ccccc7)cc65)c5ccccc45)ccc3n2)cc1 ; c1ccc(-c2nc(-c3ccccc3)c3cc(-c4cc(-n5c6cc(-c7ccccc7)c7ccccc7c6c6c7ccccc7c(-c7ccccc7)cc65)c5ccccc5c4)ccc3n2)cc1]

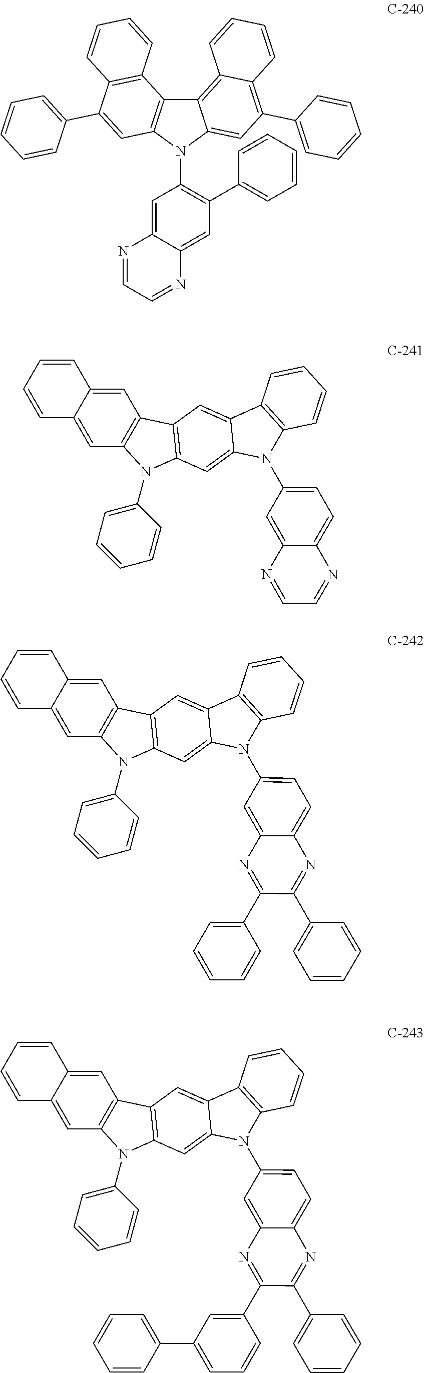 [SMILES: c1ccc(-c2cc3nccnc3cc2-n2c3cc(-c4ccccc4)c4ccccc4c3c3c4ccccc4c(-c4ccccc4)cc32)cc1 ; c1ccc(-n2c3cc4ccccc4cc3c3cc4c5ccccc5n(-c5ccc6nccnc6c5)c4cc32)cc1 ; c1ccc(-c2nc3ccc(-n4c5ccccc5c5cc6c7cc8ccccc8cc7n(-c7ccccc7)c6cc54)cc3nc2-c2ccccc2)cc1 ; c1ccc(-c2cccc(-c3nc4cc(-n5c6ccccc6c6cc7c8cc9ccccc9cc8n(-c8ccccc8)c7cc65)ccc4nc3-c3ccccc3)c2)cc1]

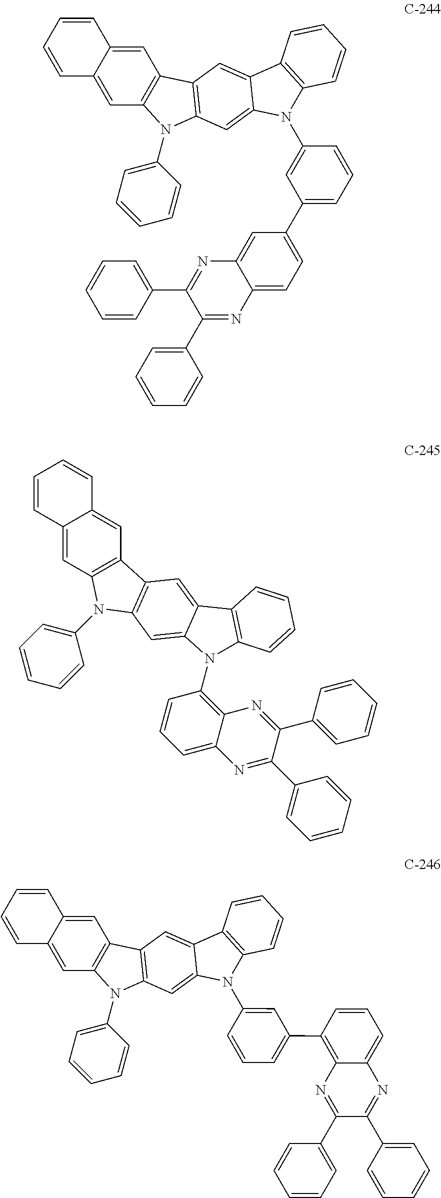 [SMILES: c1ccc(-c2nc3ccc(-c4cccc(-n5c6ccccc6c6cc7c8cc9ccccc9cc8n(-c8ccccc8)c7cc65)c4)cc3nc2-c2ccccc2)cc1 ; c1ccc(-c2nc3cccc(-n4c5ccccc5c5cc6c7cc8ccccc8cc7n(-c7ccccc7)c6cc54)c3nc2-c2ccccc2)cc1 ; c1ccc(-c2nc3cccc(-c4cccc(-n5c6ccccc6c6cc7c8cc9ccccc9cc8n(-c8ccccc8)c7cc65)c4)c3nc2-c2ccccc2)cc1]

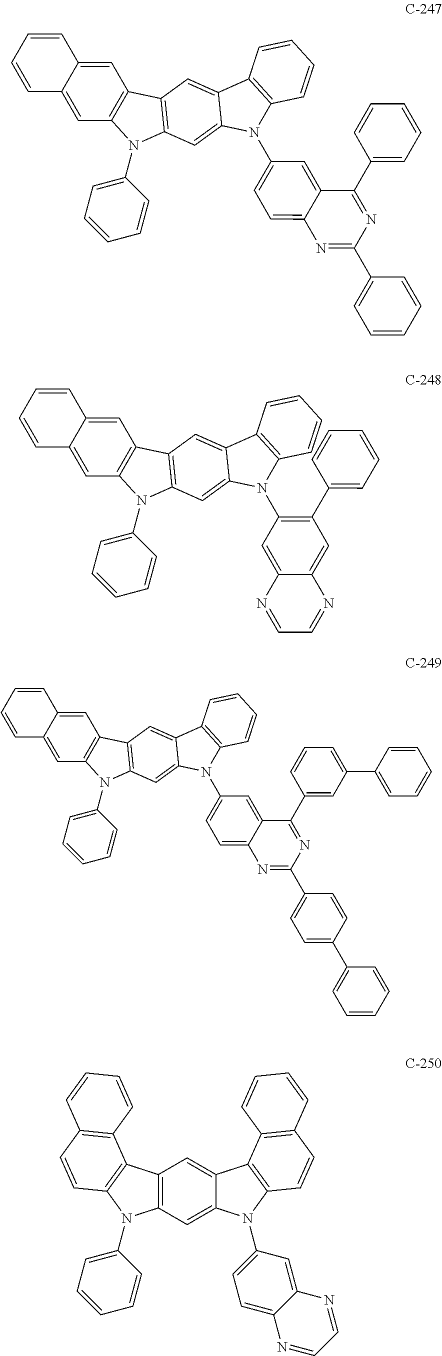 [SMILES: c1ccc(-c2nc(-c3ccccc3)c3cc(-n4c5ccccc5c5cc6c7cc8ccccc8cc7n(-c7ccccc7)c6cc54)ccc3n2)cc1 ; c1ccc(-c2cc3nccnc3cc2-n2c3ccccc3c3cc4c5cc6ccccc6cc5n(-c5ccccc5)c4cc32)cc1 ; c1ccc(-c2ccc(-c3nc(-c4cccc(-c5ccccc5)c4)c4cc(-n5c6ccccc6c6cc7c8cc9ccccc9cc8n(-c8ccccc8)c7cc65)ccc4n3)cc2)cc1 ; c1ccc(-n2c3cc4c(cc3c3c5ccccc5ccc32)c2c3ccccc3ccc2n4-c2ccc3nccnc3c2)cc1]

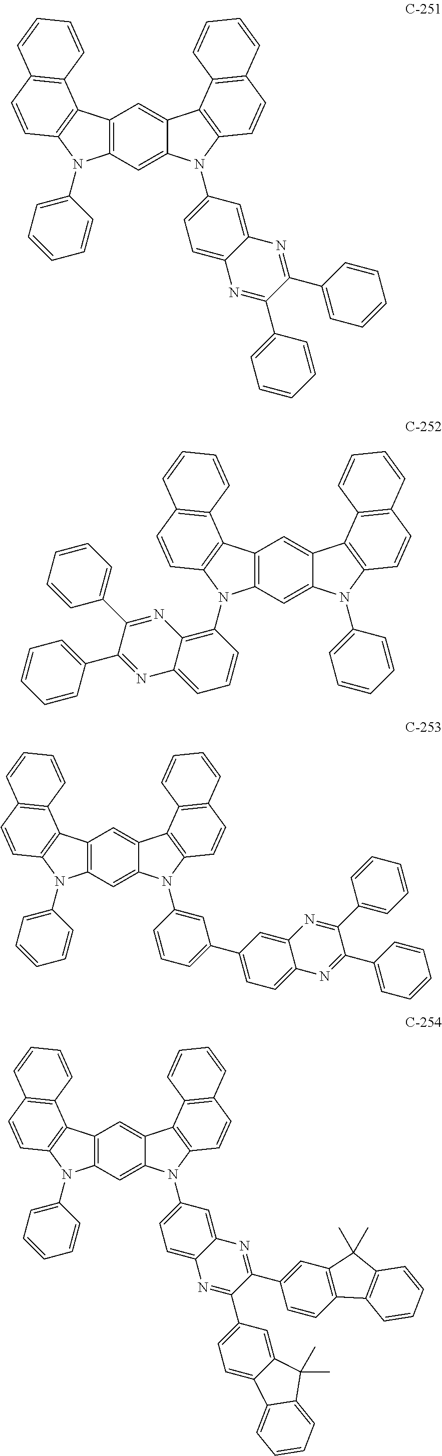 [SMILES: c1ccc(-c2nc3ccc(-n4c5cc6c(cc5c5c7ccccc7ccc54)c4c5ccccc5ccc4n6-c4ccccc4)cc3nc2-c2ccccc2)cc1 ; c1ccc(-c2nc3cccc(-n4c5cc6c(cc5c5c7ccccc7ccc54)c4c5ccccc5ccc4n6-c4ccccc4)c3nc2-c2ccccc2)cc1 ; c1ccc(-c2nc3ccc(-c4cccc(-n5c6cc7c(cc6c6c8ccccc8ccc65)c5c6ccccc6ccc5n7-c5ccccc5)c4)cc3nc2-c2ccccc2)cc1 ; CC1(C)c2ccccc2-c2ccc(-c3nc4ccc(-n5c6cc7c(cc6c6c8ccccc8ccc65)c5c6ccccc6ccc5n7-c5ccccc5)cc4nc3-c3ccc4c(c3)C(C)(C)c3ccccc3-4)cc21]

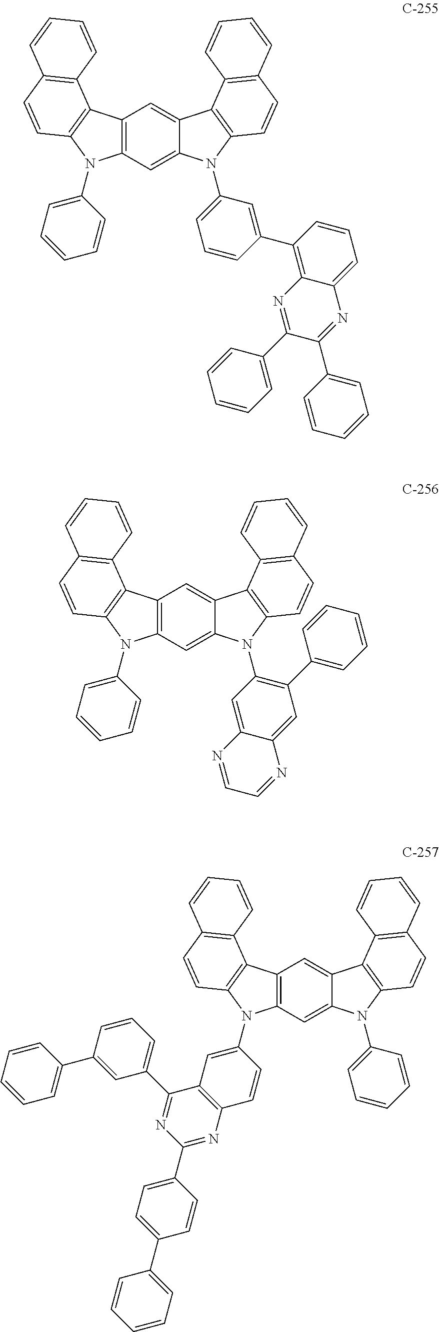 [SMILES: c1ccc(-c2nc3cccc(-c4cccc(-n5c6cc7c(cc6c6c8ccccc8ccc65)c5c6ccccc6ccc5n7-c5ccccc5)c4)c3nc2-c2ccccc2)cc1 ; c1ccc(-c2cc3nccnc3cc2-n2c3cc4c(cc3c3c5ccccc5ccc32)c2c3ccccc3ccc2n4-c2ccccc2)cc1 ; c1ccc(-c2ccc(-c3nc(-c4cccc(-c5ccccc5)c4)c4cc(-n5c6cc7c(cc6c6c8ccccc8ccc65)c5c6ccccc6ccc5n7-c5ccccc5)ccc4n3)cc2)cc1]

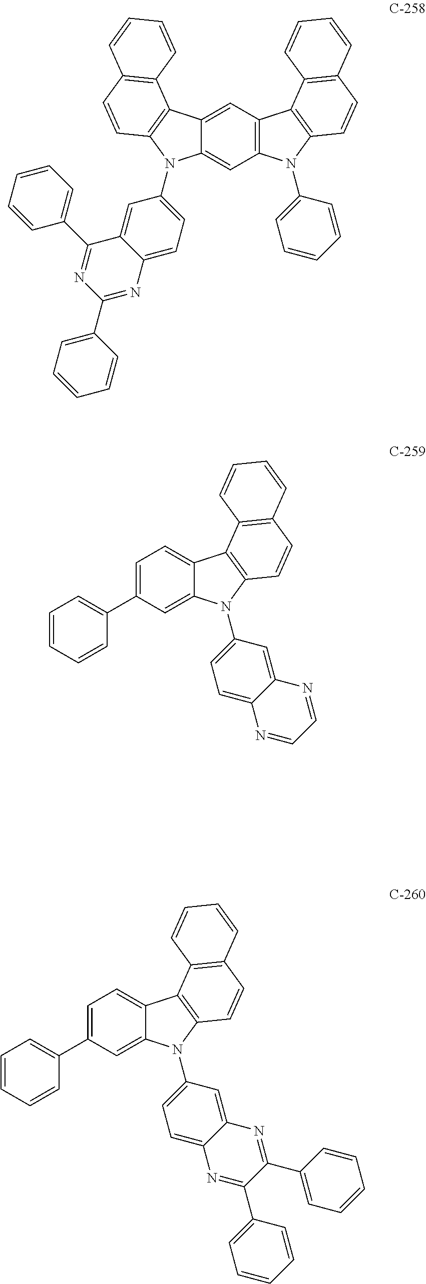 [SMILES: c1ccc(-c2nc(-c3ccccc3)c3cc(-n4c5cc6c(cc5c5c7ccccc7ccc54)c4c5ccccc5ccc4n6-c4ccccc4)ccc3n2)cc1 ; c1ccc(-c2ccc3c4c5ccccc5ccc4n(-c4ccc5nccnc5c4)c3c2)cc1 ; c1ccc(-c2ccc3c4c5ccccc5ccc4n(-c4ccc5nc(-c6ccccc6)c(-c6ccccc6)nc5c4)c3c2)cc1]

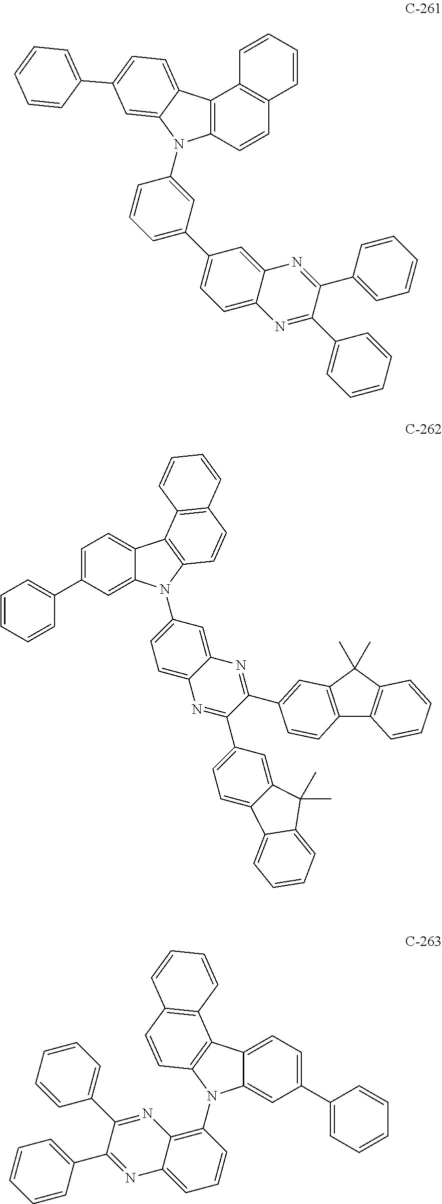 [SMILES: c1ccc(-c2ccc3c4c5ccccc5ccc4n(-c4cccc(-c5ccc6nc(-c7ccccc7)c(-c7ccccc7)nc6c5)c4)c3c2)cc1 ; CC1(C)c2ccccc2-c2ccc(-c3nc4ccc(-n5c6cc(-c7ccccc7)ccc6c6c7ccccc7ccc65)cc4nc3-c3ccc4c(c3)C(C)(C)c3ccccc3-4)cc21 ; c1ccc(-c2ccc3c4c5ccccc5ccc4n(-c4cccc5nc(-c6ccccc6)c(-c6ccccc6)nc45)c3c2)cc1]

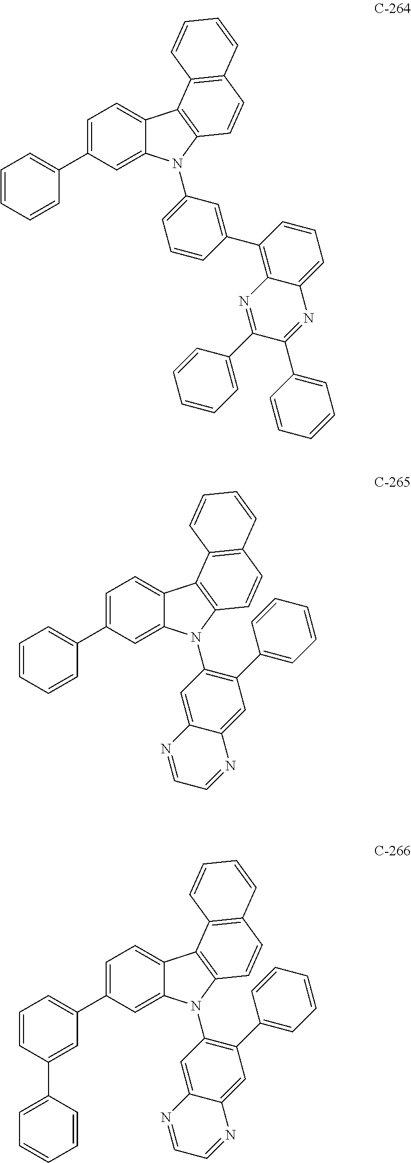 [SMILES: c1ccc(-c2ccc3c4c5ccccc5ccc4n(-c4cccc(-c5cccc6nc(-c7ccccc7)c(-c7ccccc7)nc56)c4)c3c2)cc1 ; c1ccc(-c2ccc3c4c5ccccc5ccc4n(-c4cc5nccnc5cc4-c4ccccc4)c3c2)cc1 ; c1ccc(-c2cccc(-c3ccc4c5c6ccccc6ccc5n(-c5cc6nccnc6cc5-c5ccccc5)c4c3)c2)cc1]

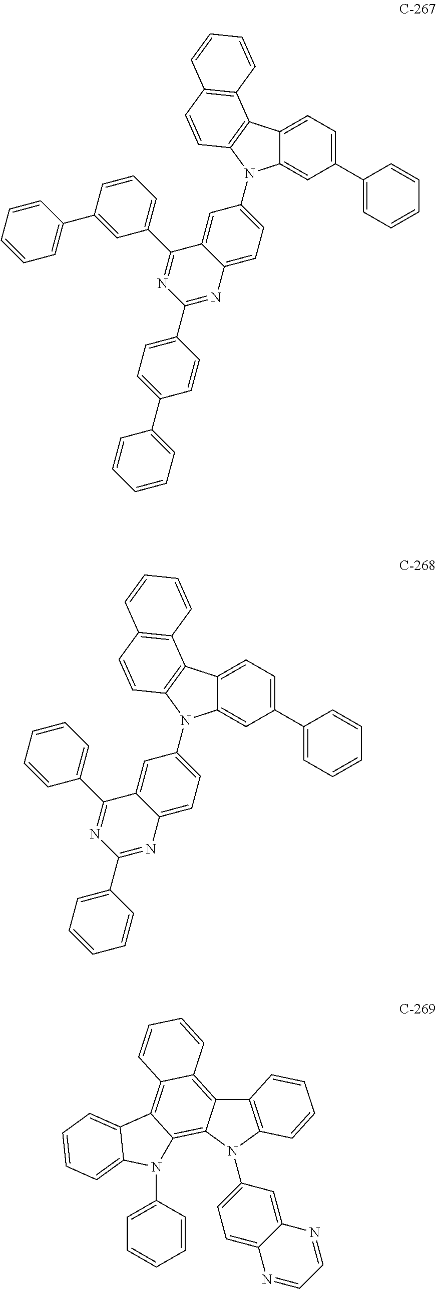 [SMILES: c1ccc(-c2ccc(-c3nc(-c4cccc(-c5ccccc5)c4)c4cc(-n5c6cc(-c7ccccc7)ccc6c6c7ccccc7ccc65)ccc4n3)cc2)cc1 ; c1ccc(-c2ccc3c4c5ccccc5ccc4n(-c4ccc5nc(-c6ccccc6)nc(-c6ccccc6)c5c4)c3c2)cc1 ; c1ccc(-n2c3ccccc3c3c4ccccc4c4c5ccccc5n(-c5ccc6nccnc6c5)c4c32)cc1]

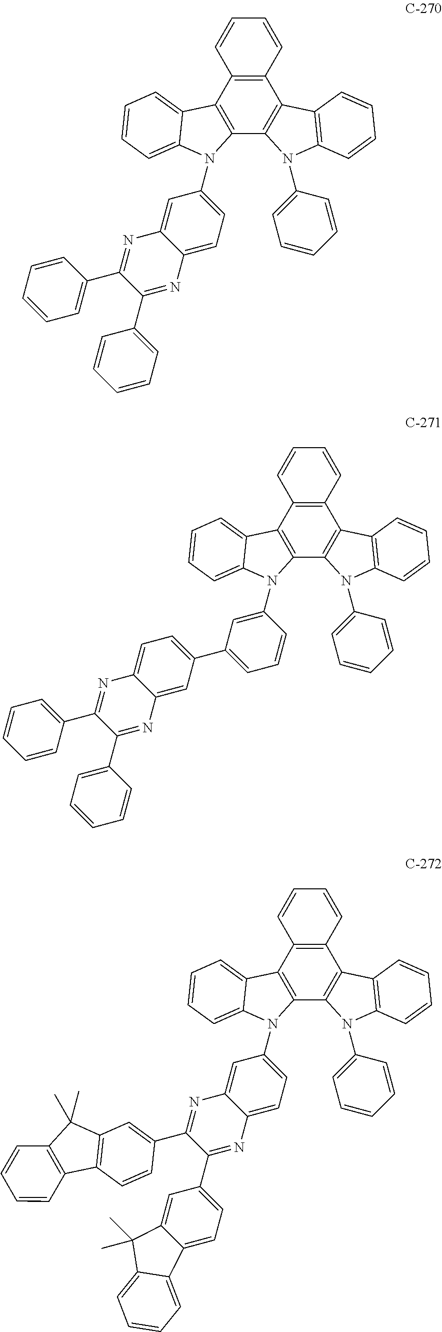 [SMILES: c1ccc(-c2nc3ccc(-n4c5ccccc5c5c6ccccc6c6c7ccccc7n(-c7ccccc7)c6c54)cc3nc2-c2ccccc2)cc1 ; c1ccc(-c2nc3ccc(-c4cccc(-n5c6ccccc6c6c7ccccc7c7c8ccccc8n(-c8ccccc8)c7c65)c4)cc3nc2-c2ccccc2)cc1 ; CC1(C)c2ccccc2-c2ccc(-c3nc4ccc(-n5c6ccccc6c6c7ccccc7c7c8ccccc8n(-c8ccccc8)c7c65)cc4nc3-c3ccc4c(c3)C(C)(C)c3ccccc3-4)cc21]

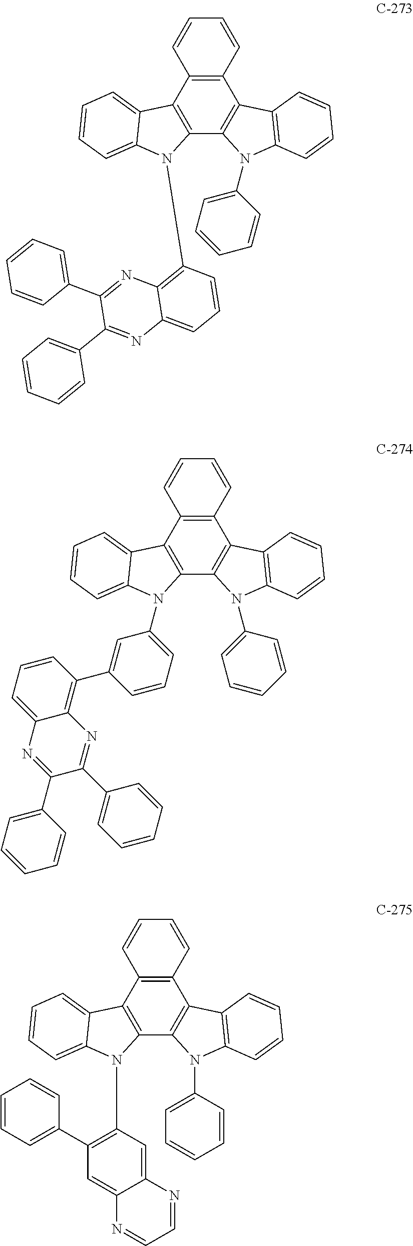 [SMILES: c1ccc(-c2nc3cccc(-n4c5ccccc5c5c6ccccc6c6c7ccccc7n(-c7ccccc7)c6c54)c3nc2-c2ccccc2)cc1 ; c1ccc(-c2nc3cccc(-c4cccc(-n5c6ccccc6c6c7ccccc7c7c8ccccc8n(-c8ccccc8)c7c65)c4)c3nc2-c2ccccc2)cc1 ; c1ccc(-c2cc3nccnc3cc2-n2c3ccccc3c3c4ccccc4c4c5ccccc5n(-c5ccccc5)c4c32)cc1]

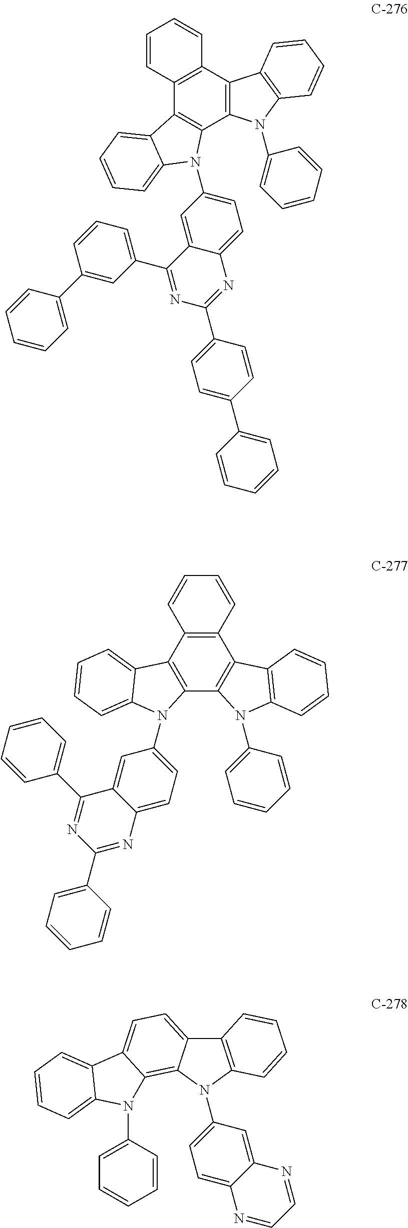 [SMILES: c1ccc(-c2ccc(-c3nc(-c4cccc(-c5ccccc5)c4)c4cc(-n5c6ccccc6c6c7ccccc7c7c8ccccc8n(-c8ccccc8)c7c65)ccc4n3)cc2)cc1 ; c1ccc(-c2nc(-c3ccccc3)c3cc(-n4c5ccccc5c5c6ccccc6c6c7ccccc7n(-c7ccccc7)c6c54)ccc3n2)cc1 ; c1ccc(-n2c3ccccc3c3ccc4c5ccccc5n(-c5ccc6nccnc6c5)c4c32)cc1]

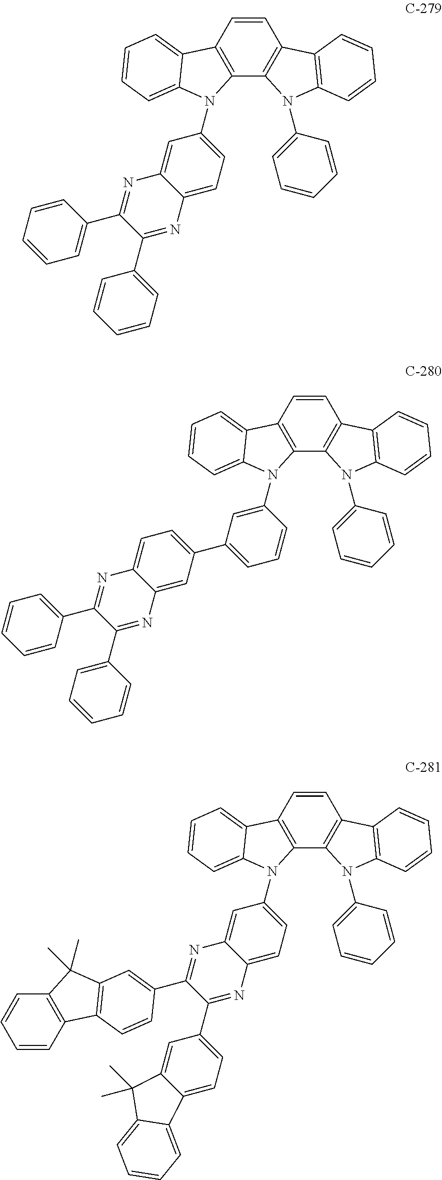 [SMILES: c1ccc(-c2nc3ccc(-n4c5ccccc5c5ccc6c7ccccc7n(-c7ccccc7)c6c54)cc3nc2-c2ccccc2)cc1 ; c1ccc(-c2nc3ccc(-c4cccc(-n5c6ccccc6c6ccc7c8ccccc8n(-c8ccccc8)c7c65)c4)cc3nc2-c2ccccc2)cc1 ; CC1(C)c2ccccc2-c2ccc(-c3nc4ccc(-n5c6ccccc6c6ccc7c8ccccc8n(-c8ccccc8)c7c65)cc4nc3-c3ccc4c(c3)C(C)(C)c3ccccc3-4)cc21]

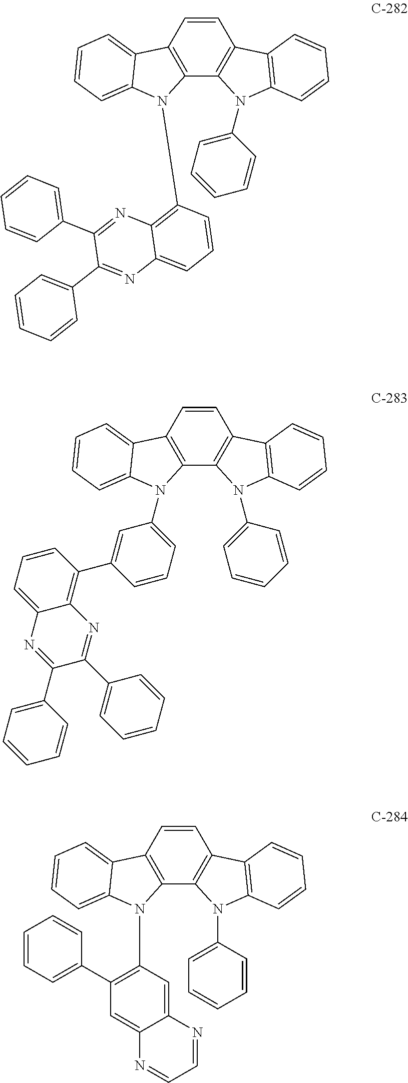 [SMILES: c1ccc(-c2nc3cccc(-n4c5ccccc5c5ccc6c7ccccc7n(-c7ccccc7)c6c54)c3nc2-c2ccccc2)cc1 ; c1ccc(-c2nc3cccc(-c4cccc(-n5c6ccccc6c6ccc7c8ccccc8n(-c8ccccc8)c7c65)c4)c3nc2-c2ccccc2)cc1 ; c1ccc(-c2cc3nccnc3cc2-n2c3ccccc3c3ccc4c5ccccc5n(-c5ccccc5)c4c32)cc1]

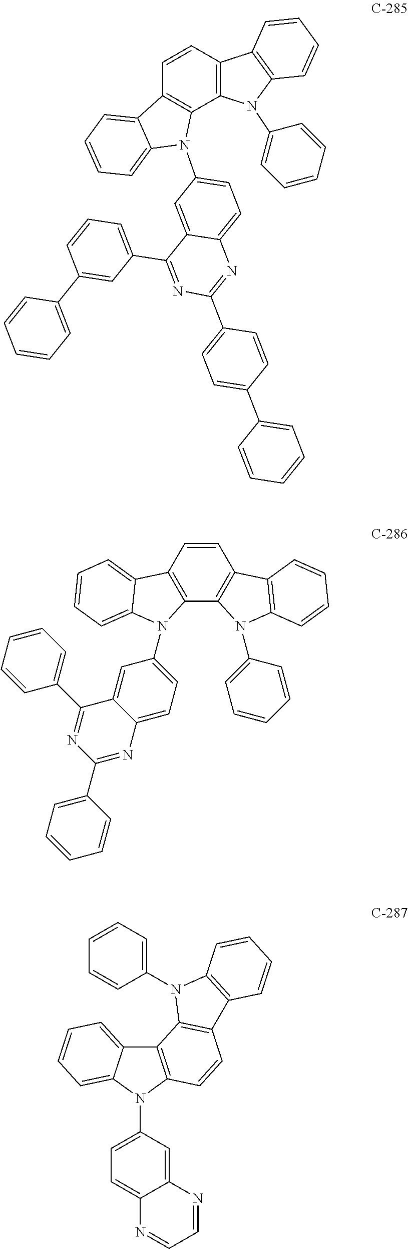 [SMILES: c1ccc(-c2ccc(-c3nc(-c4cccc(-c5ccccc5)c4)c4cc(-n5c6ccccc6c6ccc7c8ccccc8n(-c8ccccc8)c7c65)ccc4n3)cc2)cc1 ; c1ccc(-c2nc(-c3ccccc3)c3cc(-n4c5ccccc5c5ccc6c7ccccc7n(-c7ccccc7)c6c54)ccc3n2)cc1 ; c1ccc(-n2c3ccccc3c3ccc4c(c5ccccc5n4-c4ccc5nccnc5c4)c32)cc1]

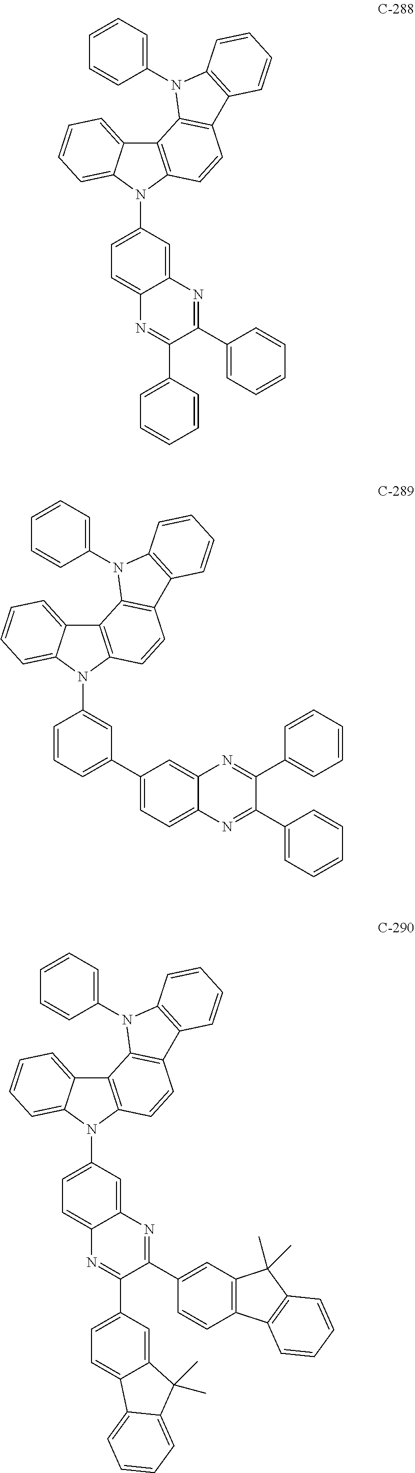 [SMILES: c1ccc(-c2nc3ccc(-n4c5ccccc5c5c4ccc4c6ccccc6n(-c6ccccc6)c45)cc3nc2-c2ccccc2)cc1 ; c1ccc(-c2nc3ccc(-c4cccc(-n5c6ccccc6c6c5ccc5c7ccccc7n(-c7ccccc7)c56)c4)cc3nc2-c2ccccc2)cc1 ; CC1(C)c2ccccc2-c2ccc(-c3nc4ccc(-n5c6ccccc6c6c5ccc5c7ccccc7n(-c7ccccc7)c56)cc4nc3-c3ccc4c(c3)C(C)(C)c3ccccc3-4)cc21]

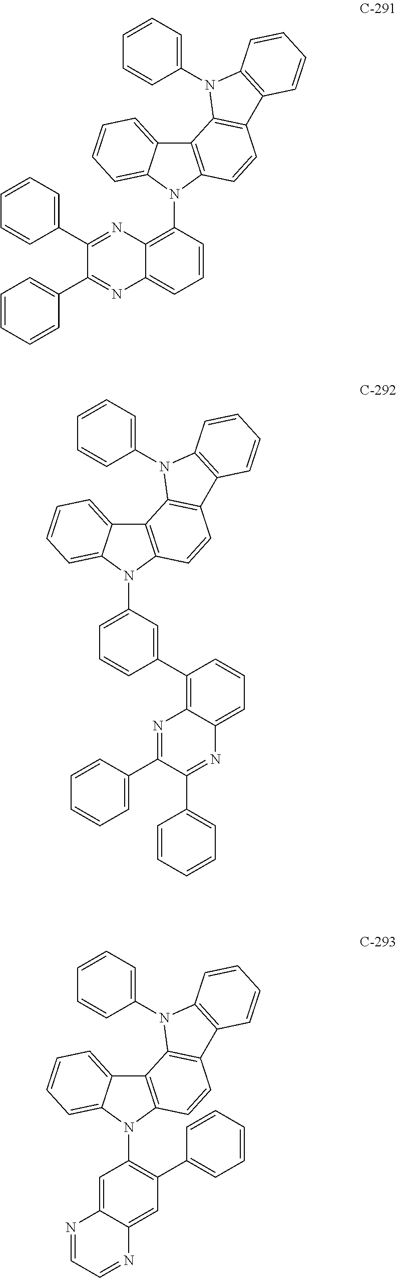 [SMILES: c1ccc(-c2nc3cccc(-n4c5ccccc5c5c4ccc4c6ccccc6n(-c6ccccc6)c45)c3nc2-c2ccccc2)cc1 ; c1ccc(-c2nc3cccc(-c4cccc(-n5c6ccccc6c6c5ccc5c7ccccc7n(-c7ccccc7)c56)c4)c3nc2-c2ccccc2)cc1 ; c1ccc(-c2cc3nccnc3cc2-n2c3ccccc3c3c2ccc2c4ccccc4n(-c4ccccc4)c23)cc1]

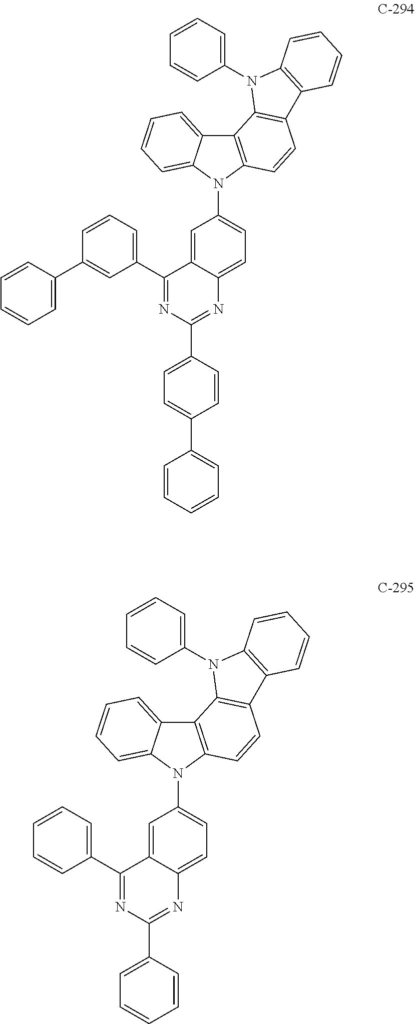 [SMILES: c1ccc(-c2ccc(-c3nc(-c4cccc(-c5ccccc5)c4)c4cc(-n5c6ccccc6c6c5ccc5c7ccccc7n(-c7ccccc7)c56)ccc4n3)cc2)cc1 ; c1ccc(-c2nc(-c3ccccc3)c3cc(-n4c5ccccc5c5c4ccc4c6ccccc6n(-c6ccccc6)c45)ccc3n2)cc1]

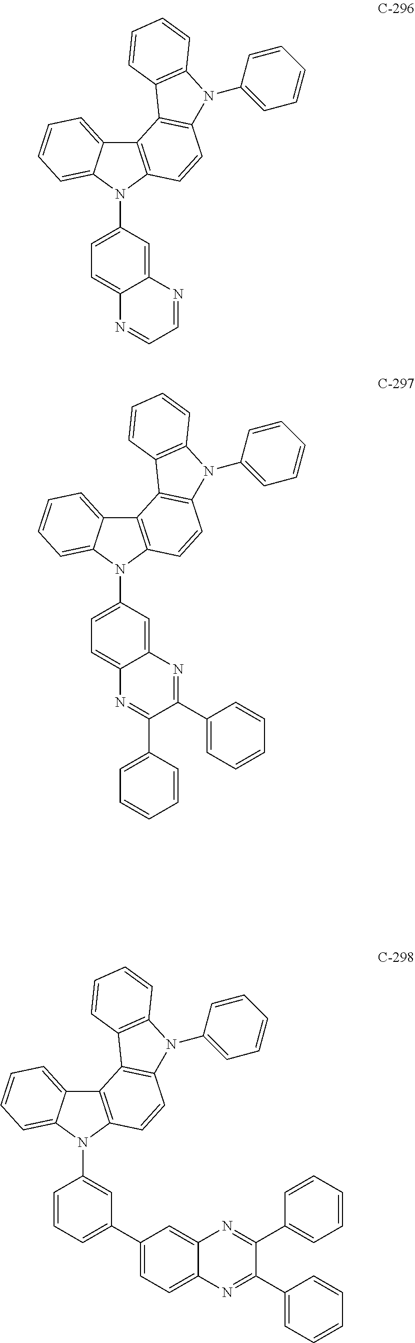 [SMILES: c1ccc(-n2c3ccccc3c3c4c5ccccc5n(-c5ccc6nccnc6c5)c4ccc32)cc1 ; c1ccc(-c2nc3ccc(-n4c5ccccc5c5c6c7ccccc7n(-c7ccccc7)c6ccc54)cc3nc2-c2ccccc2)cc1 ; c1ccc(-c2nc3ccc(-c4cccc(-n5c6ccccc6c6c7c8ccccc8n(-c8ccccc8)c7ccc65)c4)cc3nc2-c2ccccc2)cc1]

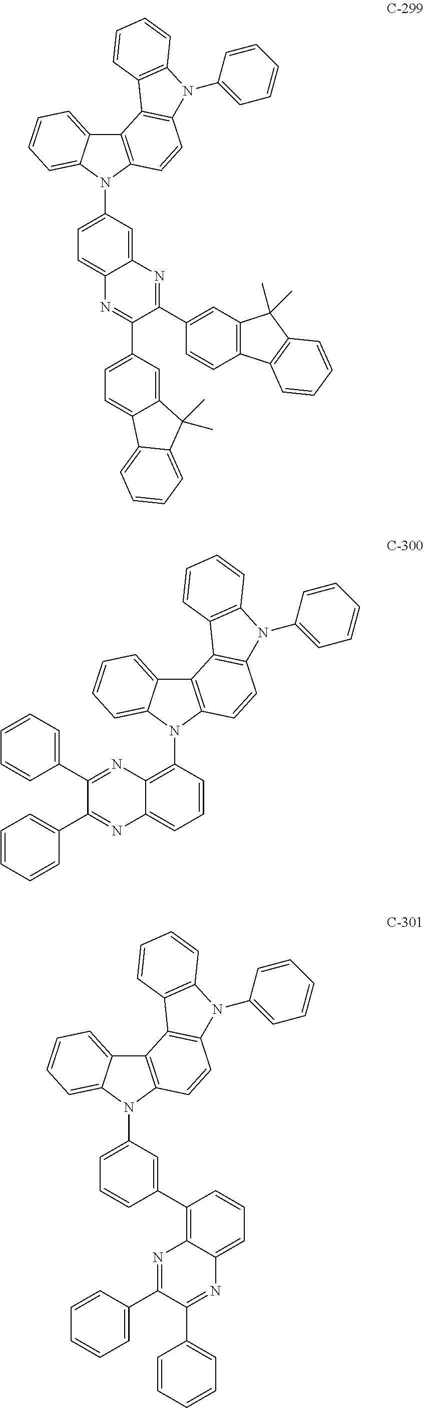 [SMILES: CC1(C)c2ccccc2-c2ccc(-c3nc4ccc(-n5c6ccccc6c6c7c8ccccc8n(-c8ccccc8)c7ccc65)cc4nc3-c3ccc4c(c3)C(C)(C)c3ccccc3-4)cc21 ; c1ccc(-c2nc3cccc(-n4c5ccccc5c5c6c7ccccc7n(-c7ccccc7)c6ccc54)c3nc2-c2ccccc2)cc1 ; c1ccc(-c2nc3cccc(-c4cccc(-n5c6ccccc6c6c7c8ccccc8n(-c8ccccc8)c7ccc65)c4)c3nc2-c2ccccc2)cc1]

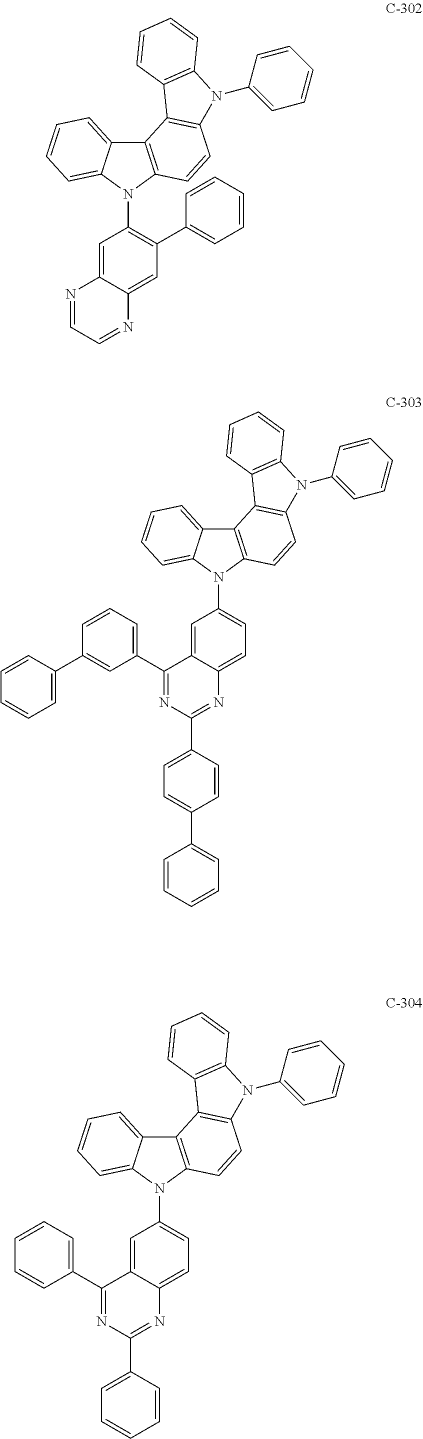 [SMILES: c1ccc(-c2cc3nccnc3cc2-n2c3ccccc3c3c4c5ccccc5n(-c5ccccc5)c4ccc32)cc1 ; c1ccc(-c2ccc(-c3nc(-c4cccc(-c5ccccc5)c4)c4cc(-n5c6ccccc6c6c7c8ccccc8n(-c8ccccc8)c7ccc65)ccc4n3)cc2)cc1 ; c1ccc(-c2nc(-c3ccccc3)c3cc(-n4c5ccccc5c5c6c7ccccc7n(-c7ccccc7)c6ccc54)ccc3n2)cc1]

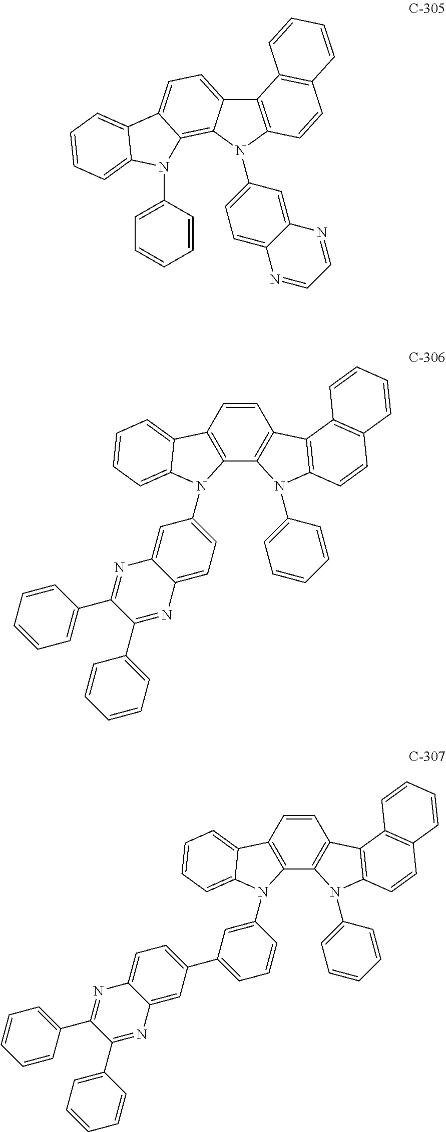 [SMILES: c1ccc(-n2c3ccccc3c3ccc4c5c6ccccc6ccc5n(-c5ccc6nccnc6c5)c4c32)cc1 ; c1ccc(-c2nc3ccc(-n4c5ccccc5c5ccc6c7c8ccccc8ccc7n(-c7ccccc7)c6c54)cc3nc2-c2ccccc2)cc1 ; c1ccc(-c2nc3ccc(-c4cccc(-n5c6ccccc6c6ccc7c8c9ccccc9ccc8n(-c8ccccc8)c7c65)c4)cc3nc2-c2ccccc2)cc1]

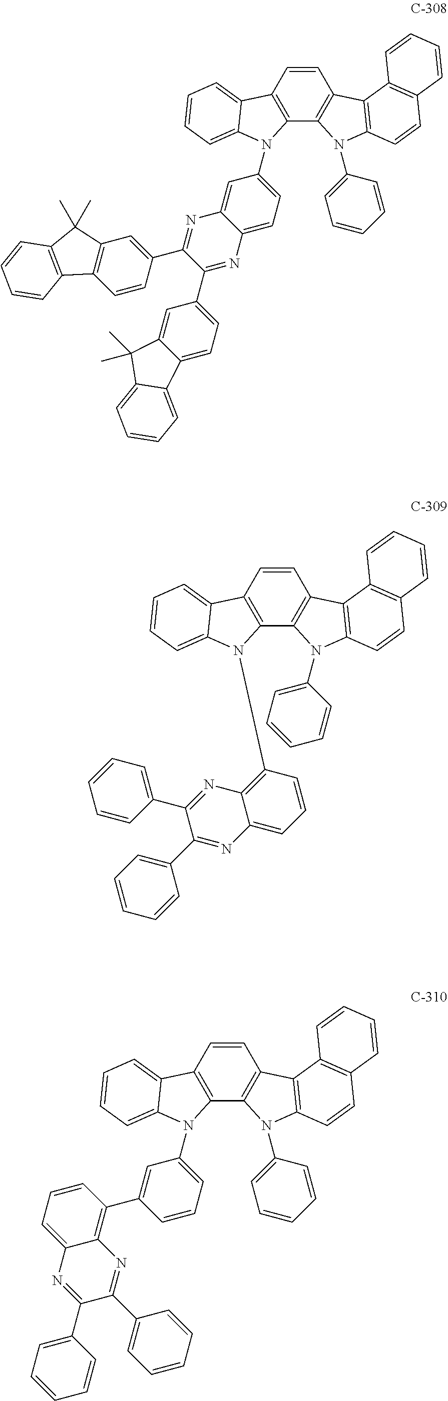

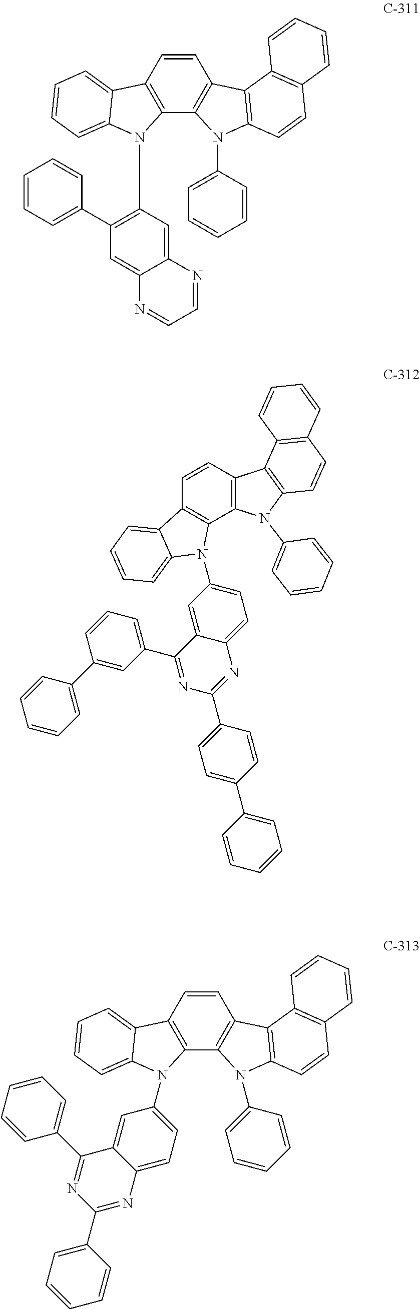 [SMILES: c1ccc(-c2cc3nccnc3cc2-n2c3ccccc3c3ccc4c5c6ccccc6ccc5n(-c5ccccc5)c4c32)cc1 ; c1ccc(-c2ccc(-c3nc(-c4cccc(-c5ccccc5)c4)c4cc(-n5c6ccccc6c6ccc7c8c9ccccc9ccc8n(-c8ccccc8)c7c65)ccc4n3)cc2)cc1 ; c1ccc(-c2nc(-c3ccccc3)c3cc(-n4c5ccccc5c5ccc6c7c8ccccc8ccc7n(-c7ccccc7)c6c54)ccc3n2)cc1]

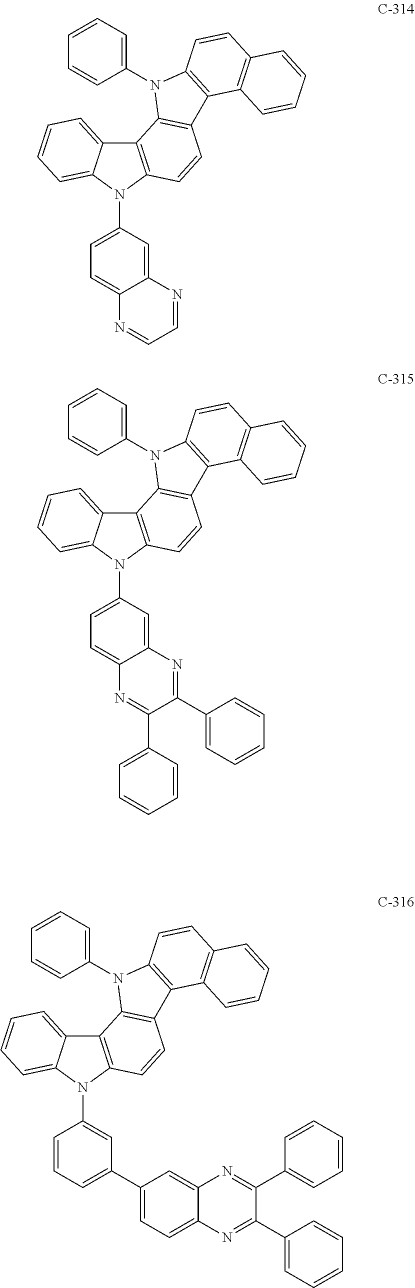 [SMILES: c1ccc(-n2c3ccc4ccccc4c3c3ccc4c(c5ccccc5n4-c4ccc5nccnc5c4)c32)cc1 ; c1ccc(-c2nc3ccc(-n4c5ccccc5c5c4ccc4c6c7ccccc7ccc6n(-c6ccccc6)c45)cc3nc2-c2ccccc2)cc1 ; c1ccc(-c2nc3ccc(-c4cccc(-n5c6ccccc6c6c5ccc5c7c8ccccc8ccc7n(-c7ccccc7)c56)c4)cc3nc2-c2ccccc2)cc1]

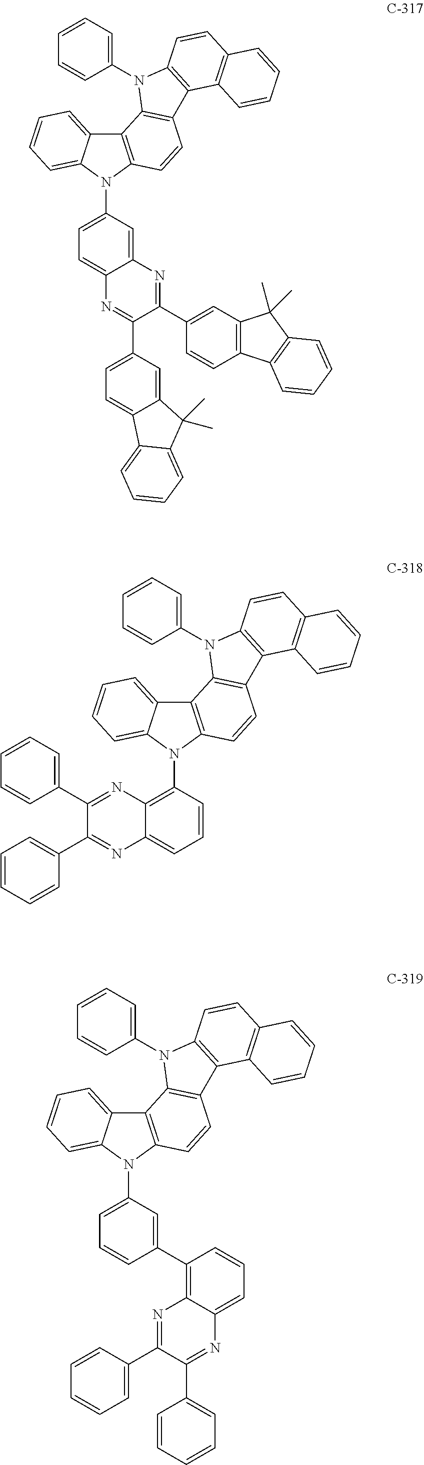 [SMILES: CC1(C)c2ccccc2-c2ccc(-c3nc4ccc(-n5c6ccccc6c6c5ccc5c7c8ccccc8ccc7n(-c7ccccc7)c56)cc4nc3-c3ccc4c(c3)C(C)(C)c3ccccc3-4)cc21 ; c1ccc(-c2nc3cccc(-n4c5ccccc5c5c4ccc4c6c7ccccc7ccc6n(-c6ccccc6)c45)c3nc2-c2ccccc2)cc1 ; c1ccc(-c2nc3cccc(-c4cccc(-n5c6ccccc6c6c5ccc5c7c8ccccc8ccc7n(-c7ccccc7)c56)c4)c3nc2-c2ccccc2)cc1]

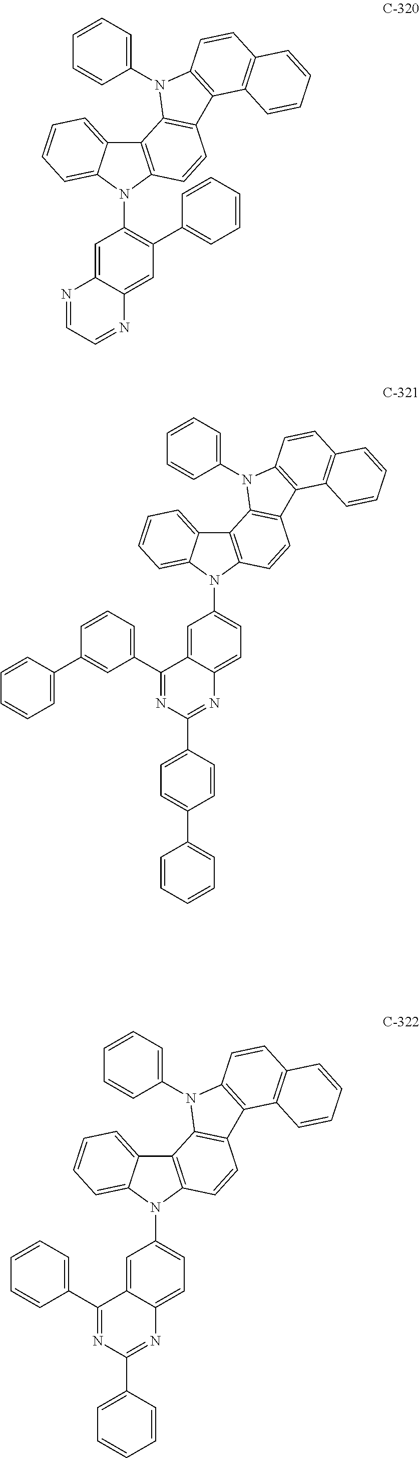 [SMILES: c1ccc(-c2cc3nccnc3cc2-n2c3ccccc3c3c2ccc2c4c5ccccc5ccc4n(-c4ccccc4)c23)cc1 ; c1ccc(-c2ccc(-c3nc(-c4cccc(-c5ccccc5)c4)c4cc(-n5c6ccccc6c6c5ccc5c7c8ccccc8ccc7n(-c7ccccc7)c56)ccc4n3)cc2)cc1 ; c1ccc(-c2nc(-c3ccccc3)c3cc(-n4c5ccccc5c5c4ccc4c6c7ccccc7ccc6n(-c6ccccc6)c45)ccc3n2)cc1]

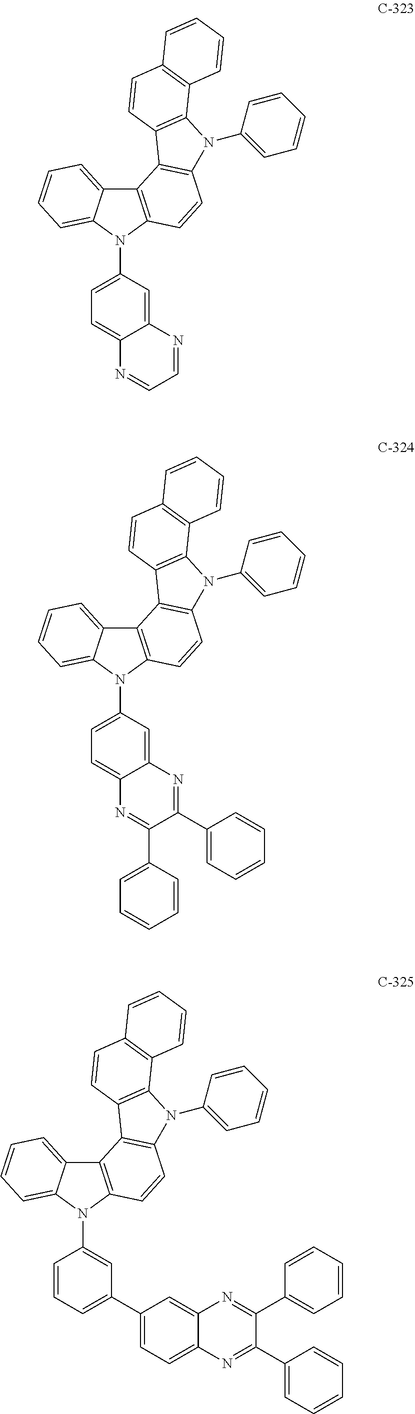 [SMILES: c1ccc(-n2c3ccc4c(c5ccccc5n4-c4ccc5nccnc5c4)c3c3ccc4ccccc4c32)cc1 ; c1ccc(-c2nc3ccc(-n4c5ccccc5c5c6c7ccc8ccccc8c7n(-c7ccccc7)c6ccc54)cc3nc2-c2ccccc2)cc1 ; c1ccc(-c2nc3ccc(-c4cccc(-n5c6ccccc6c6c7c8ccc9ccccc9c8n(-c8ccccc8)c7ccc65)c4)cc3nc2-c2ccccc2)cc1]

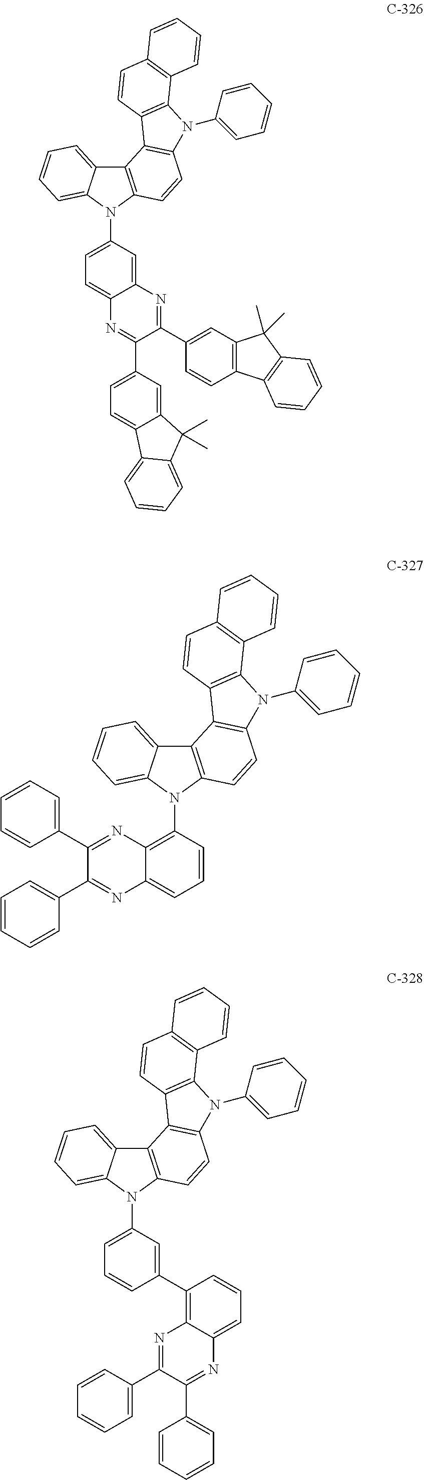 [SMILES: CC1(C)c2ccccc2-c2ccc(-c3nc4ccc(-n5c6ccccc6c6c7c8ccc9ccccc9c8n(-c8ccccc8)c7ccc65)cc4nc3-c3ccc4c(c3)C(C)(C)c3ccccc3-4)cc21 ; c1ccc(-c2nc3cccc(-n4c5ccccc5c5c6c7ccc8ccccc8c7n(-c7ccccc7)c6ccc54)c3nc2-c2ccccc2)cc1 ; c1ccc(-c2nc3cccc(-c4cccc(-n5c6ccccc6c6c7c8ccc9ccccc9c8n(-c8ccccc8)c7ccc65)c4)c3nc2-c2ccccc2)cc1]

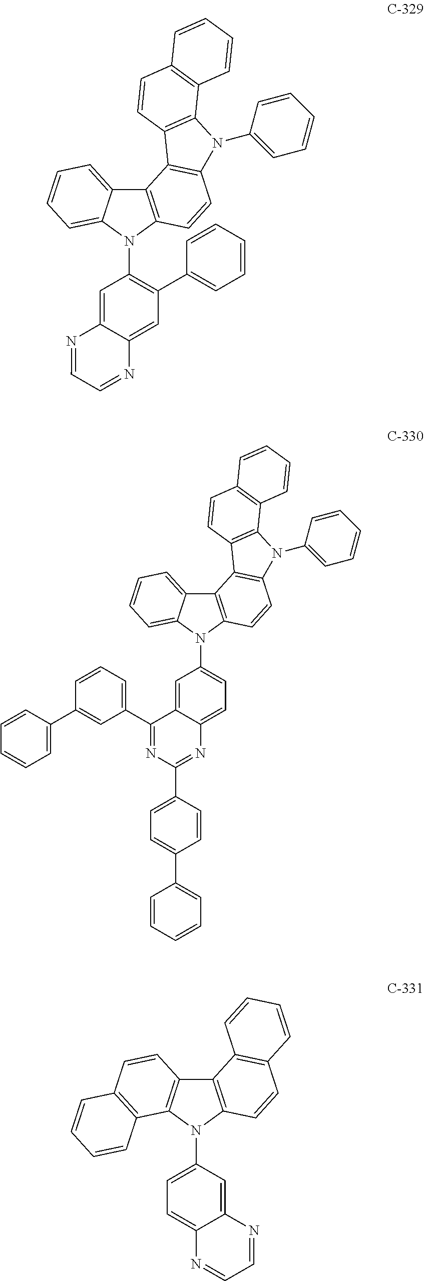 [SMILES: c1ccc(-c2cc3nccnc3cc2-n2c3ccccc3c3c4c5ccc6ccccc6c5n(-c5ccccc5)c4ccc32)cc1 ; c1ccc(-c2ccc(-c3nc(-c4cccc(-c5ccccc5)c4)c4cc(-n5c6ccccc6c6c7c8ccc9ccccc9c8n(-c8ccccc8)c7ccc65)ccc4n3)cc2)cc1 ; c1ccc2c(c1)ccc1c2c2ccc3ccccc3c2n1-c1ccc2nccnc2c1]

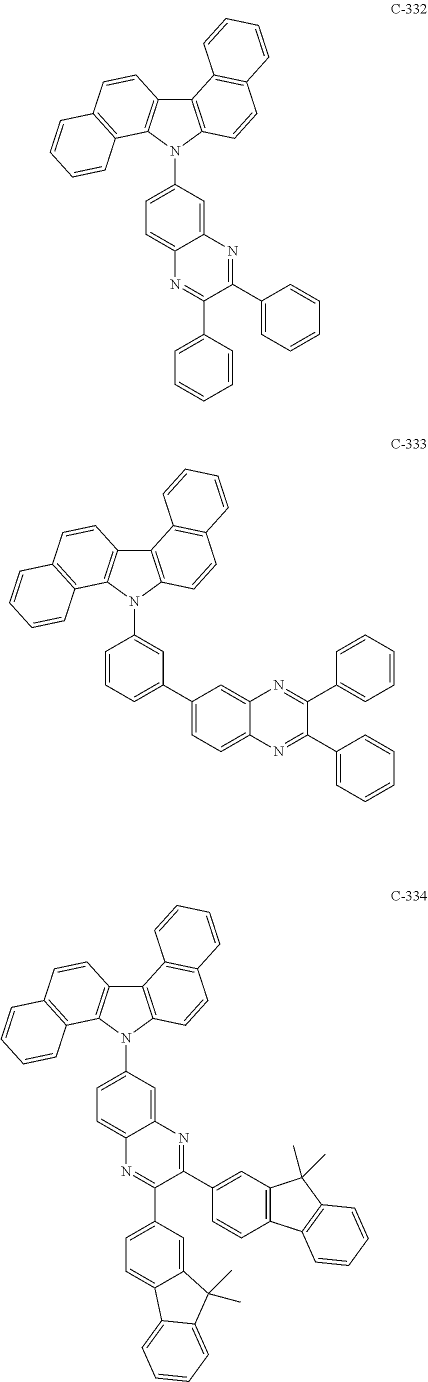 [SMILES: c1ccc(-c2nc3ccc(-n4c5ccc6ccccc6c5c5ccc6ccccc6c54)cc3nc2-c2ccccc2)cc1 ; c1ccc(-c2nc3ccc(-c4cccc(-n5c6ccc7ccccc7c6c6ccc7ccccc7c65)c4)cc3nc2-c2ccccc2)cc1 ; CC1(C)c2ccccc2-c2ccc(-c3nc4ccc(-n5c6ccc7ccccc7c6c6ccc7ccccc7c65)cc4nc3-c3ccc4c(c3)C(C)(C)c3ccccc3-4)cc21]

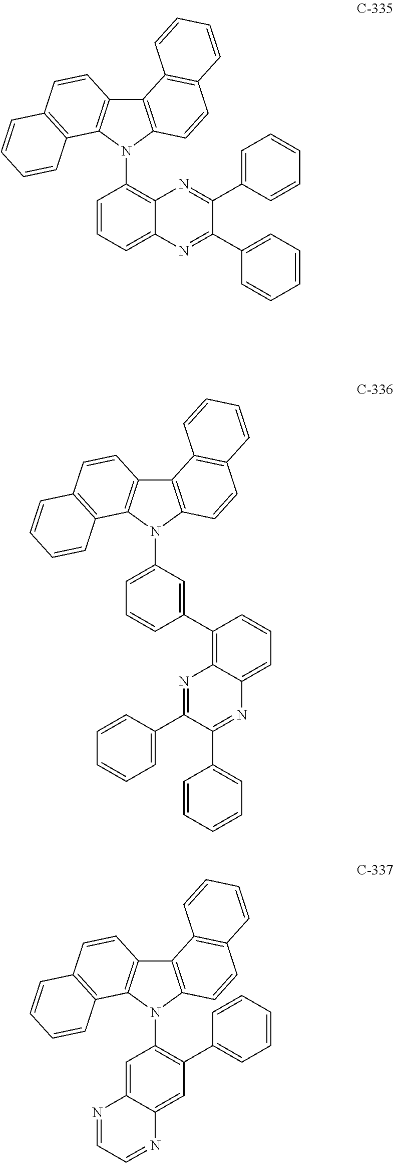 [SMILES: c1ccc(-c2nc3cccc(-n4c5ccc6ccccc6c5c5ccc6ccccc6c54)c3nc2-c2ccccc2)cc1 ; c1ccc(-c2nc3cccc(-c4cccc(-n5c6ccc7ccccc7c6c6ccc7ccccc7c65)c4)c3nc2-c2ccccc2)cc1 ; c1ccc(-c2cc3nccnc3cc2-n2c3ccc4ccccc4c3c3ccc4ccccc4c32)cc1]

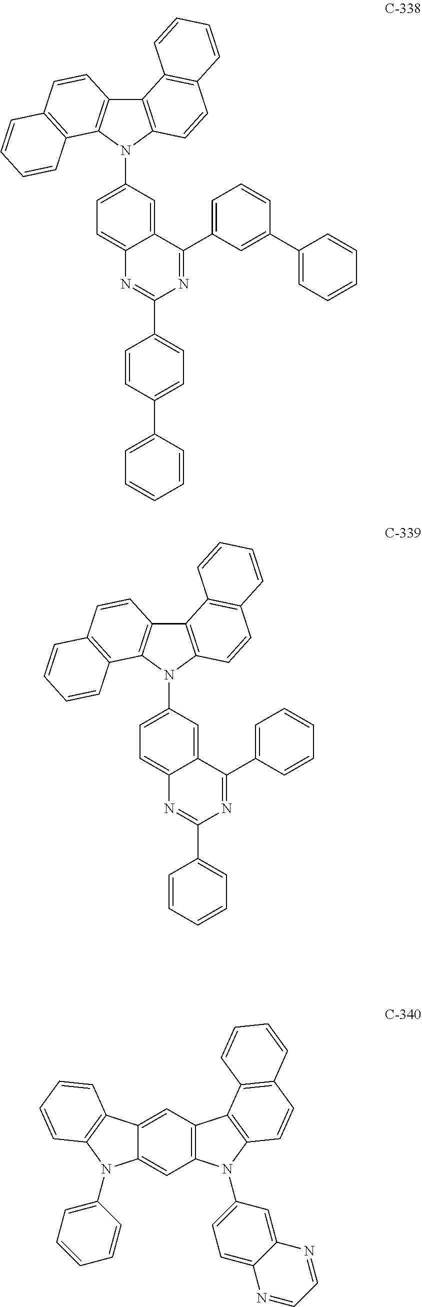 [SMILES: c1ccc(-c2ccc(-c3nc(-c4cccc(-c5ccccc5)c4)c4cc(-n5c6ccc7ccccc7c6c6ccc7ccccc7c65)ccc4n3)cc2)cc1 ; c1ccc(-c2nc(-c3ccccc3)c3cc(-n4c5ccc6ccccc6c5c5ccc6ccccc6c54)ccc3n2)cc1 ; c1ccc(-n2c3ccccc3c3cc4c5c6ccccc6ccc5n(-c5ccc6nccnc6c5)c4cc32)cc1]

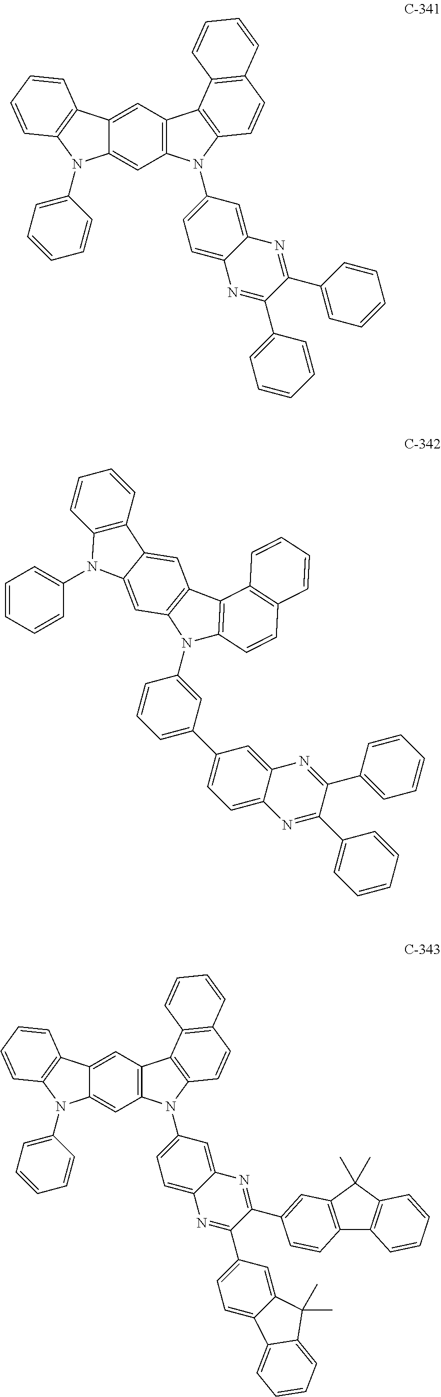 [SMILES: c1ccc(-c2nc3ccc(-n4c5cc6c(cc5c5c7ccccc7ccc54)c4ccccc4n6-c4ccccc4)cc3nc2-c2ccccc2)cc1 ; c1ccc(-c2nc3ccc(-c4cccc(-n5c6cc7c(cc6c6c8ccccc8ccc65)c5ccccc5n7-c5ccccc5)c4)cc3nc2-c2ccccc2)cc1 ; CC1(C)c2ccccc2-c2ccc(-c3nc4ccc(-n5c6cc7c(cc6c6c8ccccc8ccc65)c5ccccc5n7-c5ccccc5)cc4nc3-c3ccc4c(c3)C(C)(C)c3ccccc3-4)cc21]

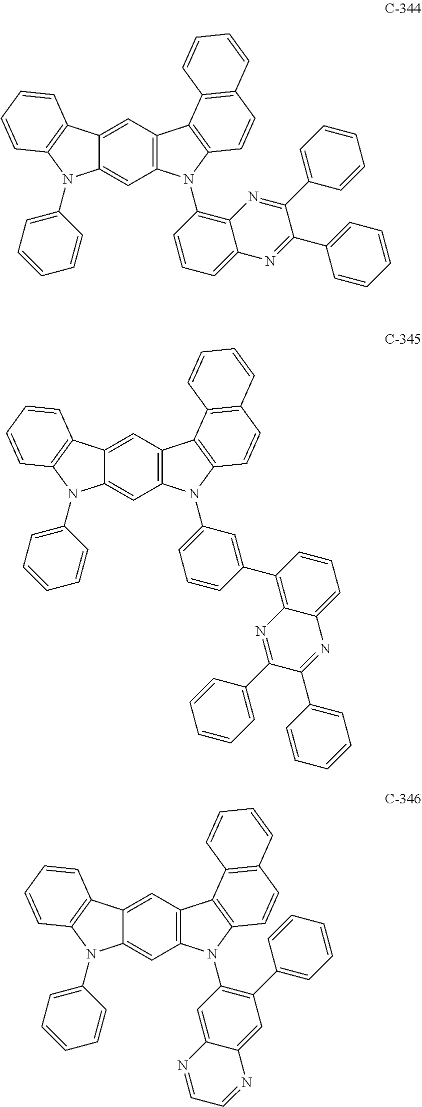 [SMILES: c1ccc(-c2nc3cccc(-n4c5cc6c(cc5c5c7ccccc7ccc54)c4ccccc4n6-c4ccccc4)c3nc2-c2ccccc2)cc1 ; c1ccc(-c2nc3cccc(-c4cccc(-n5c6cc7c(cc6c6c8ccccc8ccc65)c5ccccc5n7-c5ccccc5)c4)c3nc2-c2ccccc2)cc1 ; c1ccc(-c2cc3nccnc3cc2-n2c3cc4c(cc3c3c5ccccc5ccc32)c2ccccc2n4-c2ccccc2)cc1]

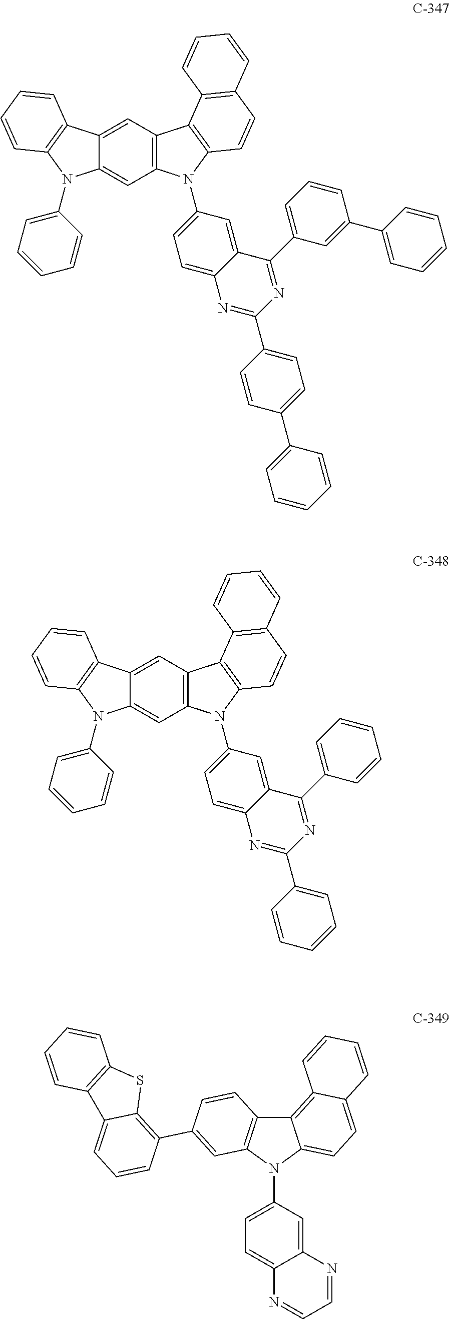 [SMILES: c1ccc(-c2ccc(-c3nc(-c4cccc(-c5ccccc5)c4)c4cc(-n5c6cc7c(cc6c6c8ccccc8ccc65)c5ccccc5n7-c5ccccc5)ccc4n3)cc2)cc1 ; c1ccc(-c2nc(-c3ccccc3)c3cc(-n4c5cc6c(cc5c5c7ccccc7ccc54)c4ccccc4n6-c4ccccc4)ccc3n2)cc1 ; c1ccc2c(c1)ccc1c2c2ccc(-c3cccc4c3sc3ccccc34)cc2n1-c1ccc2nccnc2c1]

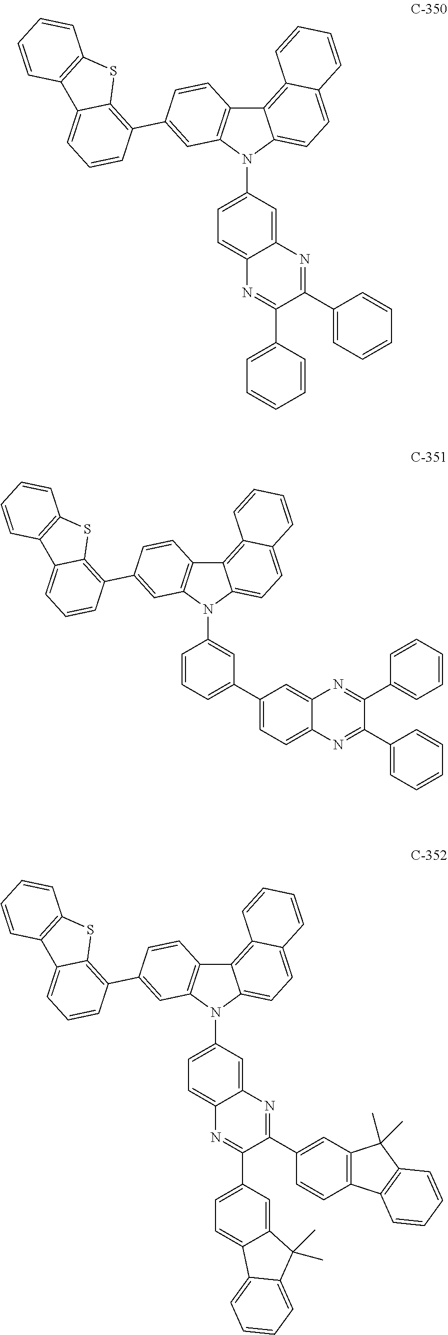 [SMILES: c1ccc(-c2nc3ccc(-n4c5cc(-c6cccc7c6sc6ccccc67)ccc5c5c6ccccc6ccc54)cc3nc2-c2ccccc2)cc1 ; c1ccc(-c2nc3ccc(-c4cccc(-n5c6cc(-c7cccc8c7sc7ccccc78)ccc6c6c7ccccc7ccc65)c4)cc3nc2-c2ccccc2)cc1 ; CC1(C)c2ccccc2-c2ccc(-c3nc4ccc(-n5c6cc(-c7cccc8c7sc7ccccc78)ccc6c6c7ccccc7ccc65)cc4nc3-c3ccc4c(c3)C(C)(C)c3ccccc3-4)cc21]

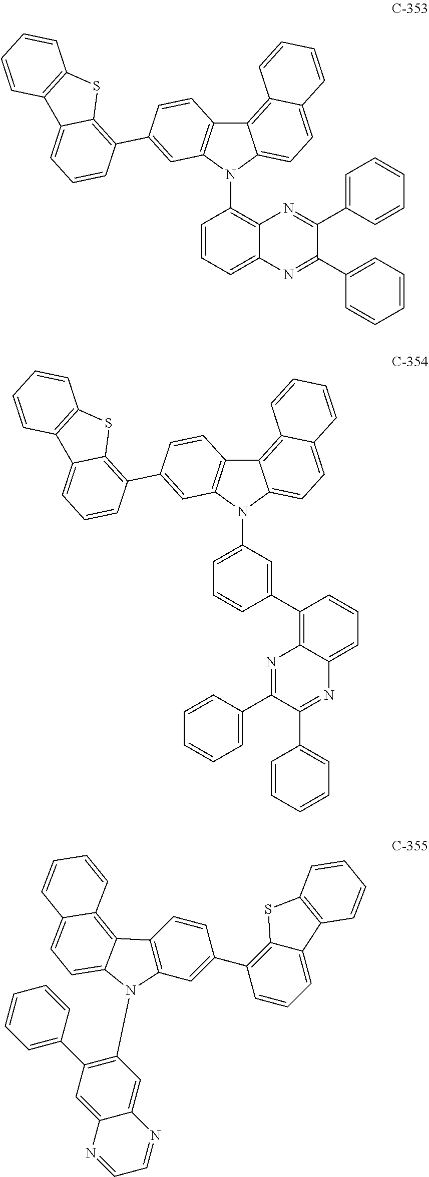 [SMILES: c1ccc(-c2nc3cccc(-n4c5cc(-c6cccc7c6sc6ccccc67)ccc5c5c6ccccc6ccc54)c3nc2-c2ccccc2)cc1 ; c1ccc(-c2nc3cccc(-c4cccc(-n5c6cc(-c7cccc8c7sc7ccccc78)ccc6c6c7ccccc7ccc65)c4)c3nc2-c2ccccc2)cc1 ; c1ccc(-c2cc3nccnc3cc2-n2c3cc(-c4cccc5c4sc4ccccc45)ccc3c3c4ccccc4ccc32)cc1]

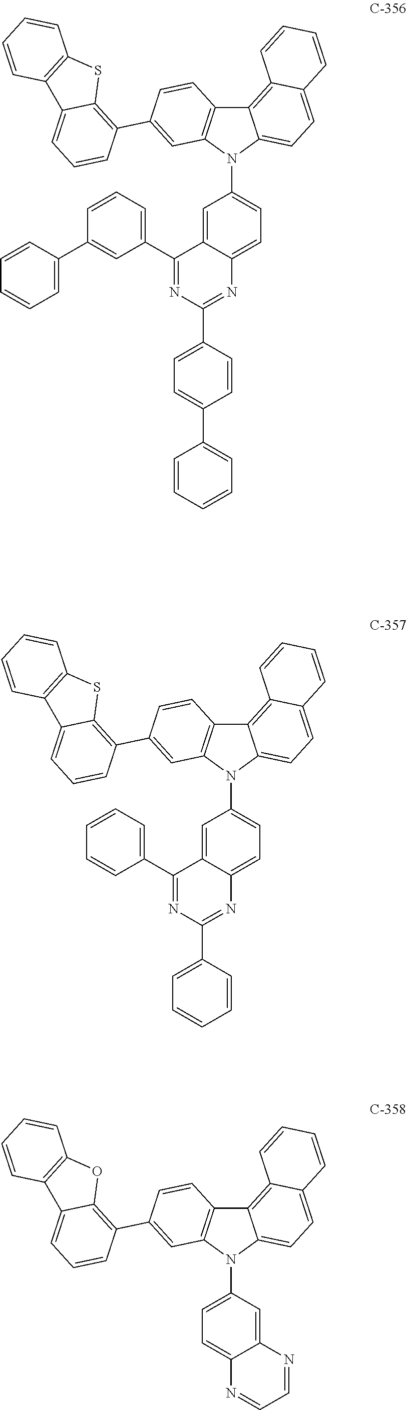 [SMILES: c1ccc(-c2ccc(-c3nc(-c4cccc(-c5ccccc5)c4)c4cc(-n5c6cc(-c7cccc8c7sc7ccccc78)ccc6c6c7ccccc7ccc65)ccc4n3)cc2)cc1 ; c1ccc(-c2nc(-c3ccccc3)c3cc(-n4c5cc(-c6cccc7c6sc6ccccc67)ccc5c5c6ccccc6ccc54)ccc3n2)cc1 ; c1ccc2c(c1)ccc1c2c2ccc(-c3cccc4c3oc3ccccc34)cc2n1-c1ccc2nccnc2c1]

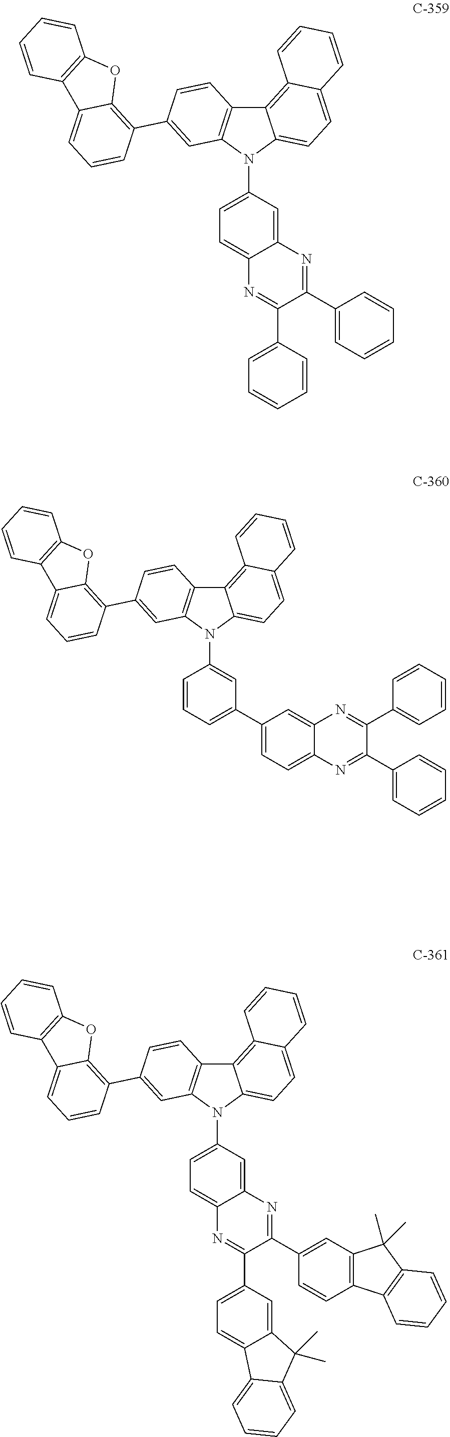 [SMILES: c1ccc(-c2nc3ccc(-n4c5cc(-c6cccc7c6oc6ccccc67)ccc5c5c6ccccc6ccc54)cc3nc2-c2ccccc2)cc1 ; c1ccc(-c2nc3ccc(-c4cccc(-n5c6cc(-c7cccc8c7oc7ccccc78)ccc6c6c7ccccc7ccc65)c4)cc3nc2-c2ccccc2)cc1 ; CC1(C)c2ccccc2-c2ccc(-c3nc4ccc(-n5c6cc(-c7cccc8c7oc7ccccc78)ccc6c6c7ccccc7ccc65)cc4nc3-c3ccc4c(c3)C(C)(C)c3ccccc3-4)cc21]

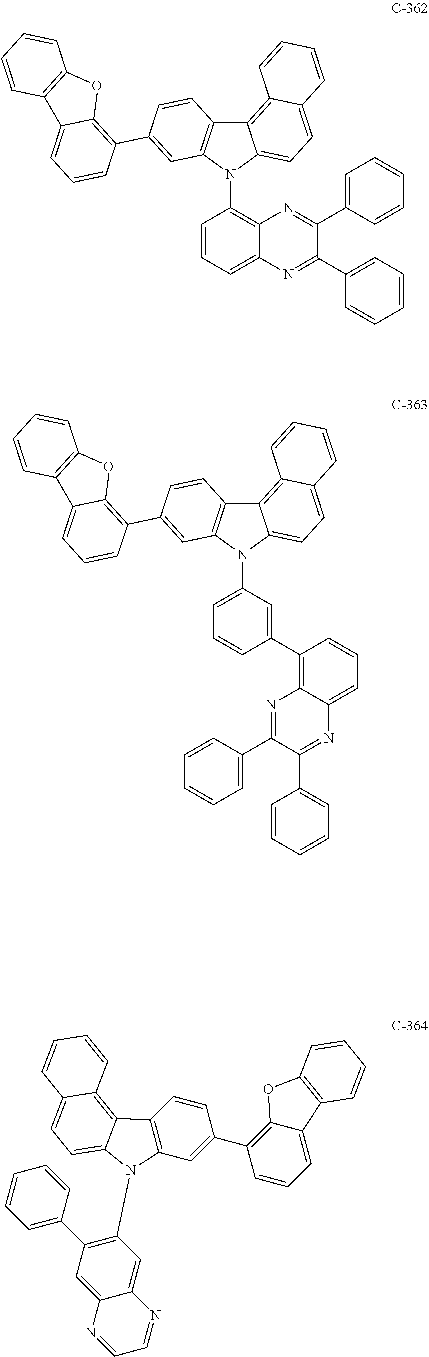 [SMILES: c1ccc(-c2nc3cccc(-n4c5cc(-c6cccc7c6oc6ccccc67)ccc5c5c6ccccc6ccc54)c3nc2-c2ccccc2)cc1 ; c1ccc(-c2nc3cccc(-c4cccc(-n5c6cc(-c7cccc8c7oc7ccccc78)ccc6c6c7ccccc7ccc65)c4)c3nc2-c2ccccc2)cc1 ; c1ccc(-c2cc3nccnc3cc2-n2c3cc(-c4cccc5c4oc4ccccc45)ccc3c3c4ccccc4ccc32)cc1]

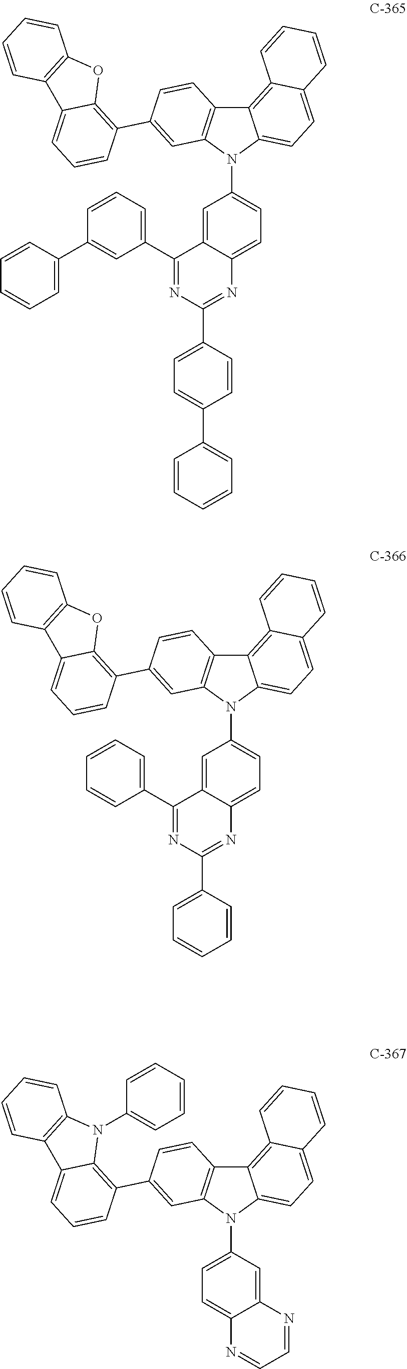 [SMILES: c1ccc(-c2ccc(-c3nc(-c4cccc(-c5ccccc5)c4)c4cc(-n5c6cc(-c7cccc8c7oc7ccccc78)ccc6c6c7ccccc7ccc65)ccc4n3)cc2)cc1 ; c1ccc(-c2nc(-c3ccccc3)c3cc(-n4c5cc(-c6cccc7c6oc6ccccc67)ccc5c5c6ccccc6ccc54)ccc3n2)cc1 ; c1ccc(-n2c3ccccc3c3cccc(-c4ccc5c6c7ccccc7ccc6n(-c6ccc7nccnc7c6)c5c4)c32)cc1]

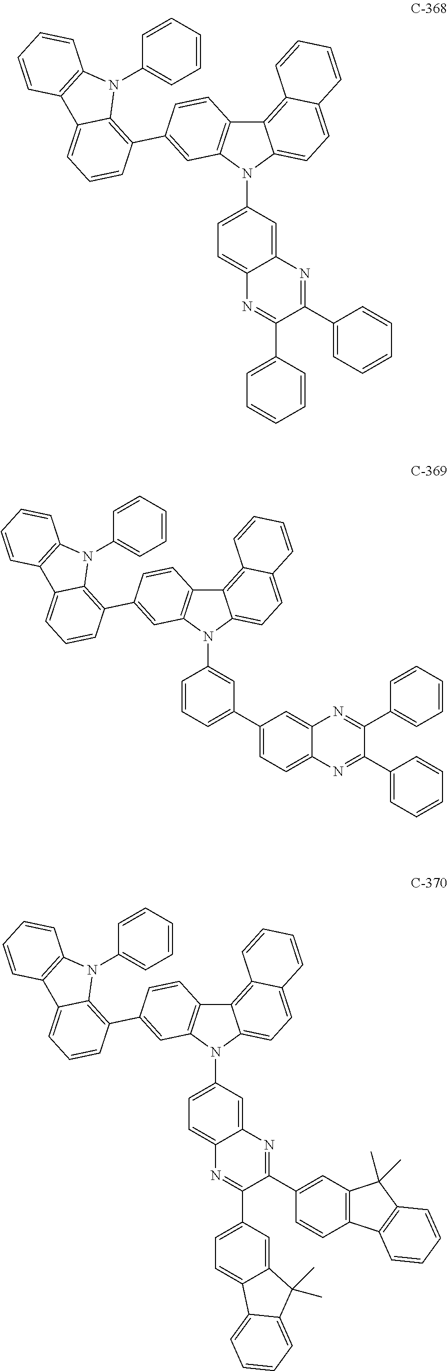 [SMILES: c1ccc(-c2nc3ccc(-n4c5cc(-c6cccc7c8ccccc8n(-c8ccccc8)c67)ccc5c5c6ccccc6ccc54)cc3nc2-c2ccccc2)cc1 ; c1ccc(-c2nc3ccc(-c4cccc(-n5c6cc(-c7cccc8c9ccccc9n(-c9ccccc9)c78)ccc6c6c7ccccc7ccc65)c4)cc3nc2-c2ccccc2)cc1 ; CC1(C)c2ccccc2-c2ccc(-c3nc4ccc(-n5c6cc(-c7cccc8c9ccccc9n(-c9ccccc9)c78)ccc6c6c7ccccc7ccc65)cc4nc3-c3ccc4c(c3)C(C)(C)c3ccccc3-4)cc21]

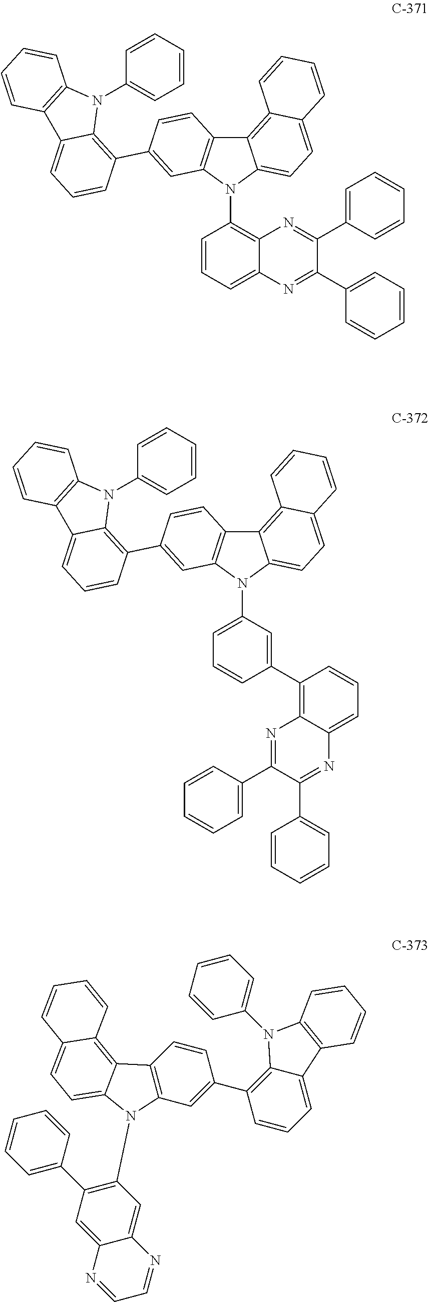 [SMILES: c1ccc(-c2nc3cccc(-n4c5cc(-c6cccc7c8ccccc8n(-c8ccccc8)c67)ccc5c5c6ccccc6ccc54)c3nc2-c2ccccc2)cc1 ; c1ccc(-c2nc3cccc(-c4cccc(-n5c6cc(-c7cccc8c9ccccc9n(-c9ccccc9)c78)ccc6c6c7ccccc7ccc65)c4)c3nc2-c2ccccc2)cc1 ; c1ccc(-c2cc3nccnc3cc2-n2c3cc(-c4cccc5c6ccccc6n(-c6ccccc6)c45)ccc3c3c4ccccc4ccc32)cc1]

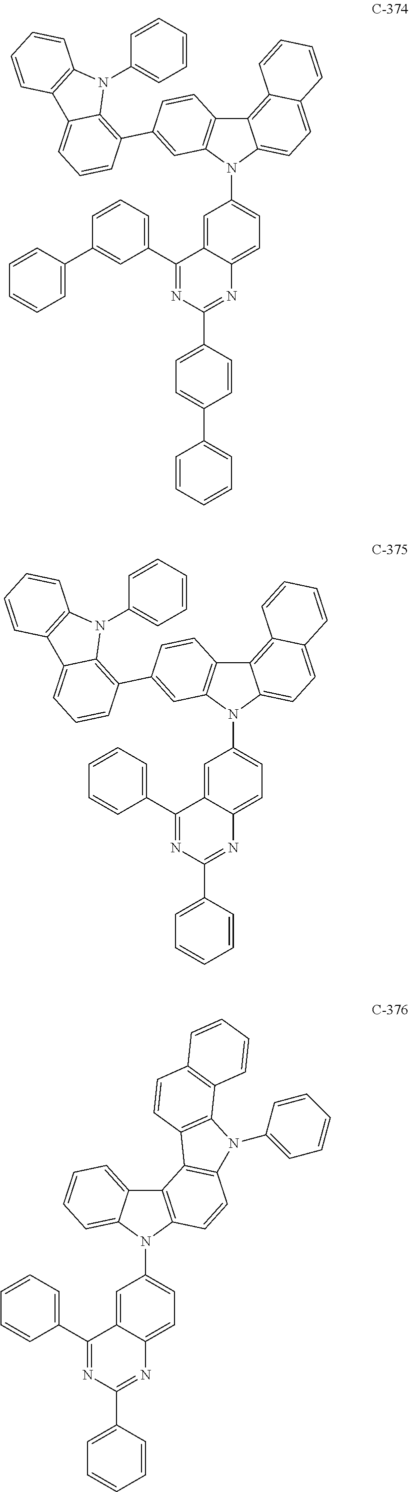 [SMILES: c1ccc(-c2ccc(-c3nc(-c4cccc(-c5ccccc5)c4)c4cc(-n5c6cc(-c7cccc8c9ccccc9n(-c9ccccc9)c78)ccc6c6c7ccccc7ccc65)ccc4n3)cc2)cc1 ; c1ccc(-c2nc(-c3ccccc3)c3cc(-n4c5cc(-c6cccc7c8ccccc8n(-c8ccccc8)c67)ccc5c5c6ccccc6ccc54)ccc3n2)cc1 ; c1ccc(-c2nc(-c3ccccc3)c3cc(-n4c5ccccc5c5c6c7ccc8ccccc8c7n(-c7ccccc7)c6ccc54)ccc3n2)cc1]

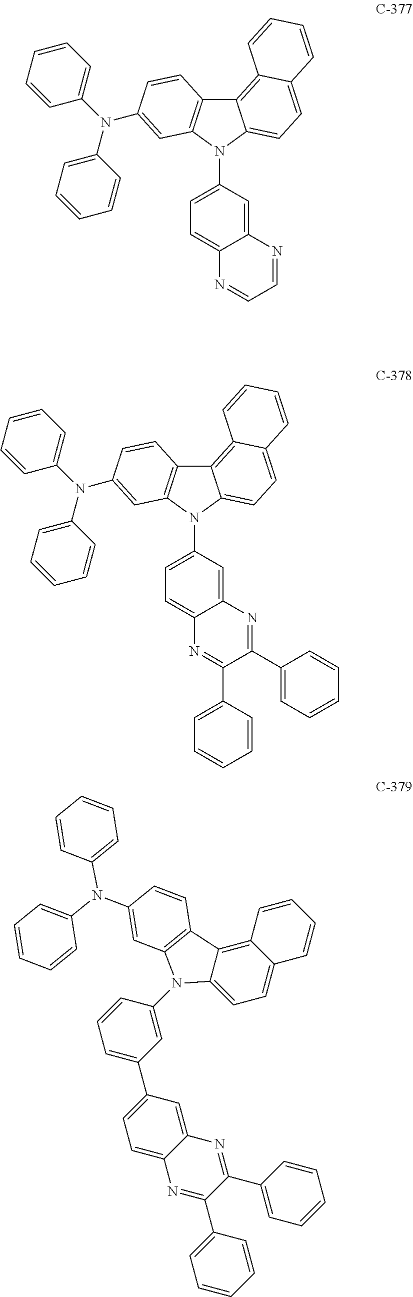 [SMILES: c1ccc(N(c2ccccc2)c2ccc3c4c5ccccc5ccc4n(-c4ccc5nccnc5c4)c3c2)cc1 ; c1ccc(-c2nc3ccc(-n4c5cc(N(c6ccccc6)c6ccccc6)ccc5c5c6ccccc6ccc54)cc3nc2-c2ccccc2)cc1 ; c1ccc(-c2nc3ccc(-c4cccc(-n5c6cc(N(c7ccccc7)c7ccccc7)ccc6c6c7ccccc7ccc65)c4)cc3nc2-c2ccccc2)cc1]

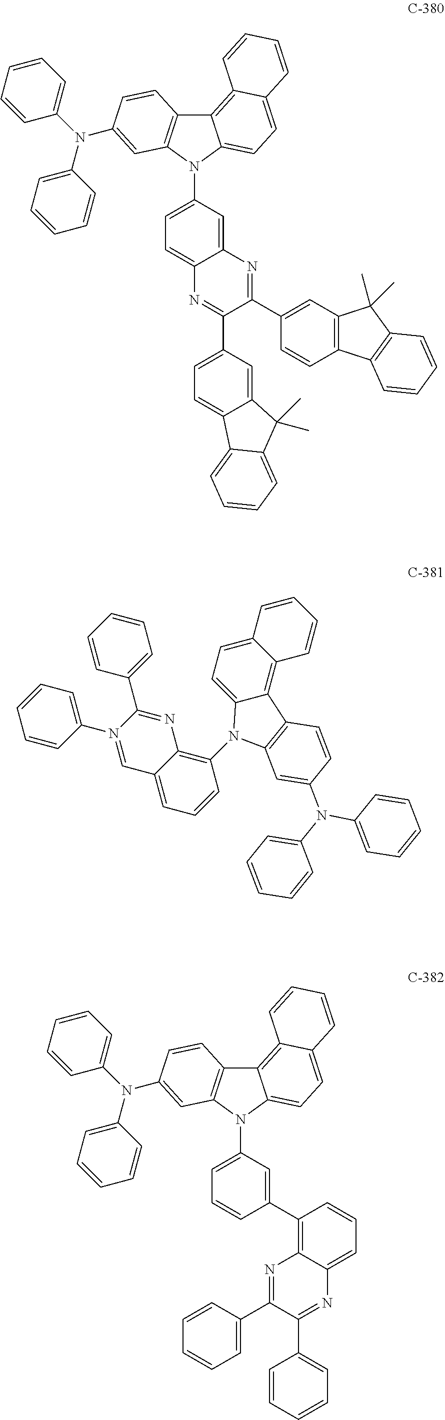

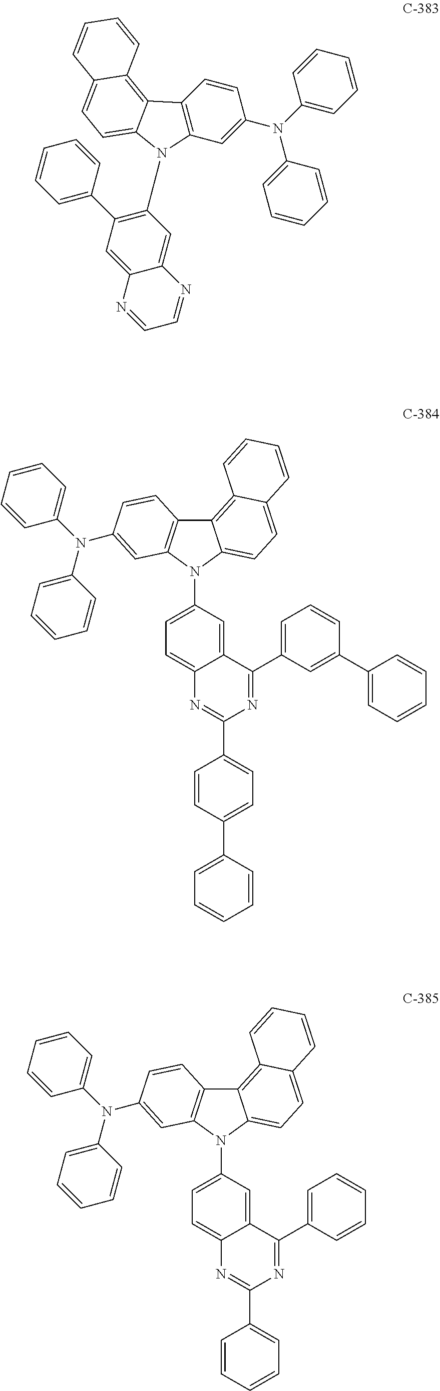 [SMILES: c1ccc(-c2cc3nccnc3cc2-n2c3cc(N(c4ccccc4)c4ccccc4)ccc3c3c4ccccc4ccc32)cc1 ; c1ccc(-c2ccc(-c3nc(-c4cccc(-c5ccccc5)c4)c4cc(-n5c6cc(N(c7ccccc7)c7ccccc7)ccc6c6c7ccccc7ccc65)ccc4n3)cc2)cc1 ; c1ccc(-c2nc(-c3ccccc3)c3cc(-n4c5cc(N(c6ccccc6)c6ccccc6)ccc5c5c6ccccc6ccc54)ccc3n2)cc1]

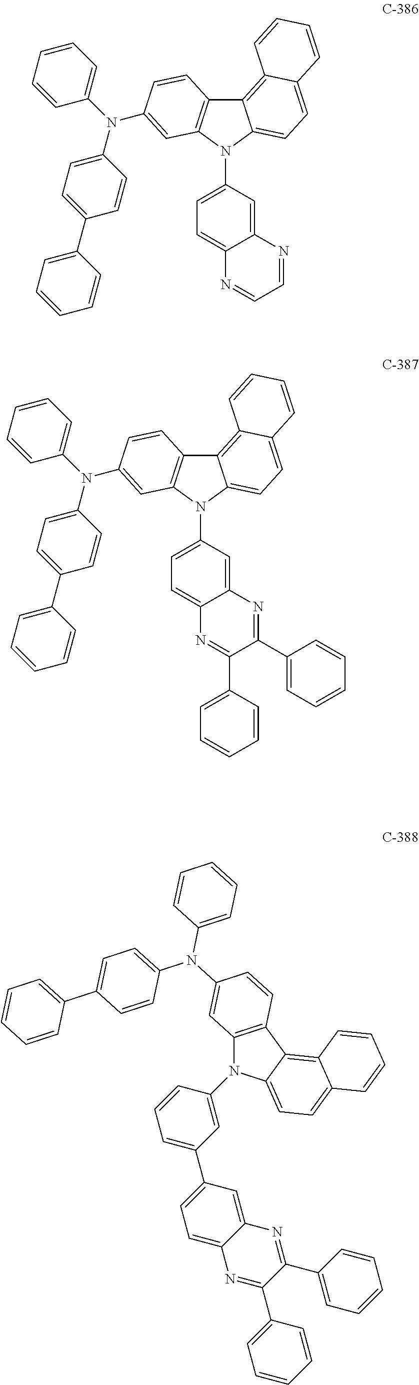 [SMILES: c1ccc(-c2ccc(N(c3ccccc3)c3ccc4c5c6ccccc6ccc5n(-c5ccc6nccnc6c5)c4c3)cc2)cc1 ; c1ccc(-c2ccc(N(c3ccccc3)c3ccc4c5c6ccccc6ccc5n(-c5ccc6nc(-c7ccccc7)c(-c7ccccc7)nc6c5)c4c3)cc2)cc1 ; c1ccc(-c2ccc(N(c3ccccc3)c3ccc4c5c6ccccc6ccc5n(-c5cccc(-c6ccc7nc(-c8ccccc8)c(-c8ccccc8)nc7c6)c5)c4c3)cc2)cc1]

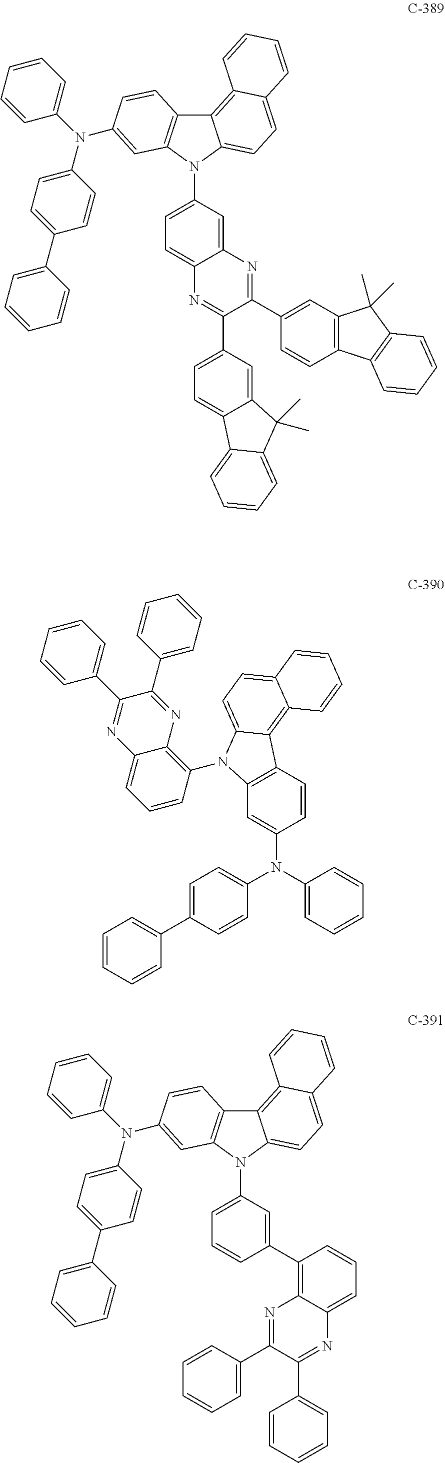 [SMILES: CC1(C)c2ccccc2-c2ccc(-c3nc4ccc(-n5c6cc(N(c7ccccc7)c7ccc(-c8ccccc8)cc7)ccc6c6c7ccccc7ccc65)cc4nc3-c3ccc4c(c3)C(C)(C)c3ccccc3-4)cc21 ; c1ccc(-c2ccc(N(c3ccccc3)c3ccc4c5c6ccccc6ccc5n(-c5cccc6nc(-c7ccccc7)c(-c7ccccc7)nc56)c4c3)cc2)cc1 ; c1ccc(-c2ccc(N(c3ccccc3)c3ccc4c5c6ccccc6ccc5n(-c5cccc(-c6cccc7nc(-c8ccccc8)c(-c8ccccc8)nc67)c5)c4c3)cc2)cc1]

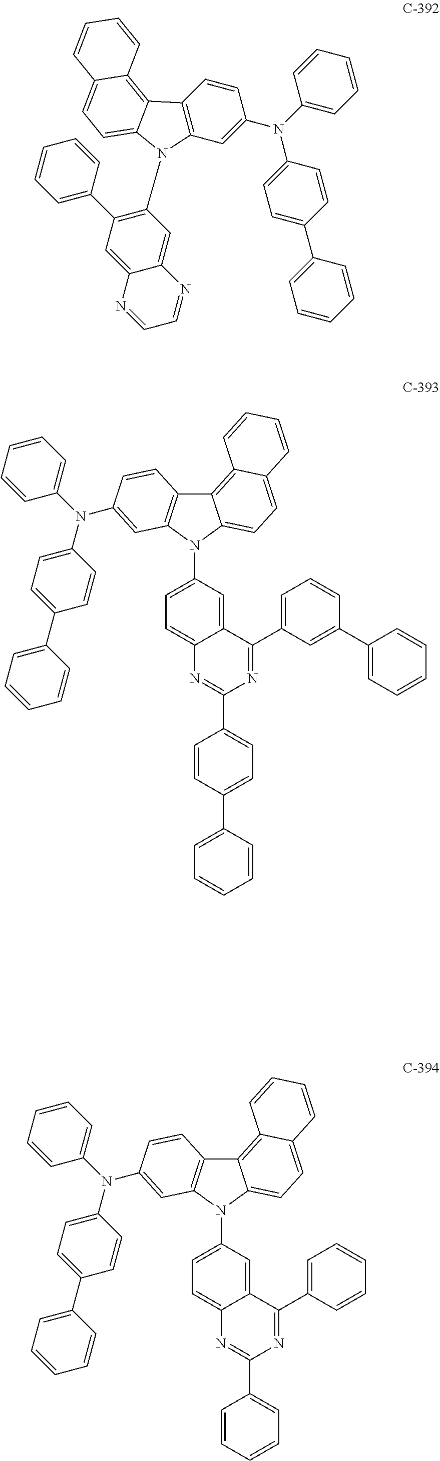 [SMILES: c1ccc(-c2ccc(N(c3ccccc3)c3ccc4c5c6ccccc6ccc5n(-c5cc6nccnc6cc5-c5ccccc5)c4c3)cc2)cc1 ; c1ccc(-c2ccc(-c3nc(-c4cccc(-c5ccccc5)c4)c4cc(-n5c6cc(N(c7ccccc7)c7ccc(-c8ccccc8)cc7)ccc6c6c7ccccc7ccc65)ccc4n3)cc2)cc1 ; c1ccc(-c2ccc(N(c3ccccc3)c3ccc4c5c6ccccc6ccc5n(-c5ccc6nc(-c7ccccc7)nc(-c7ccccc7)c6c5)c4c3)cc2)cc1]

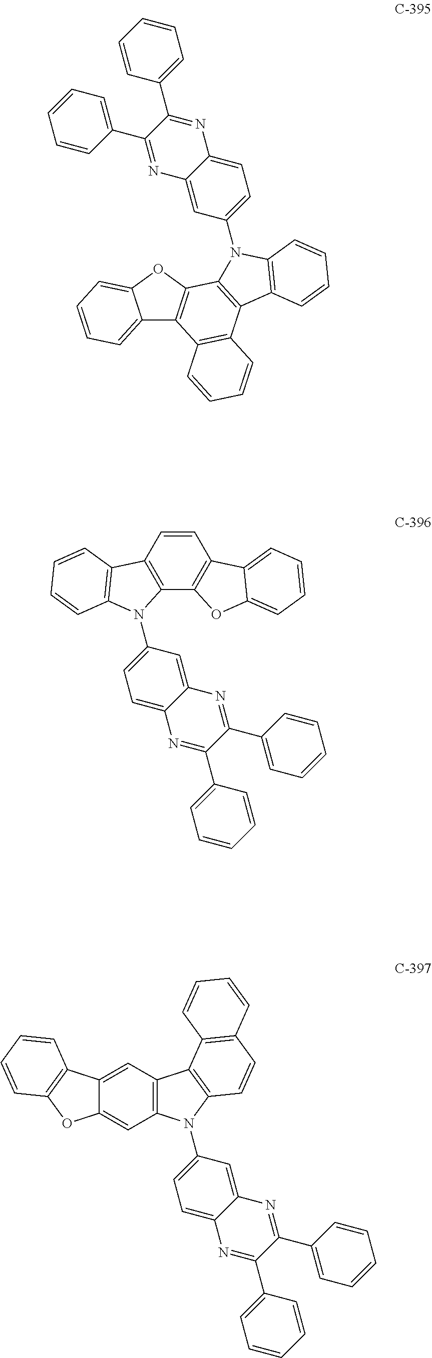 [SMILES: c1ccc(-c2nc3ccc(-n4c5ccccc5c5c6ccccc6c6c7ccccc7oc6c54)cc3nc2-c2ccccc2)cc1 ; c1ccc(-c2nc3ccc(-n4c5ccccc5c5ccc6c7ccccc7oc6c54)cc3nc2-c2ccccc2)cc1 ; c1ccc(-c2nc3ccc(-n4c5cc6oc7ccccc7c6cc5c5c6ccccc6ccc54)cc3nc2-c2ccccc2)cc1]

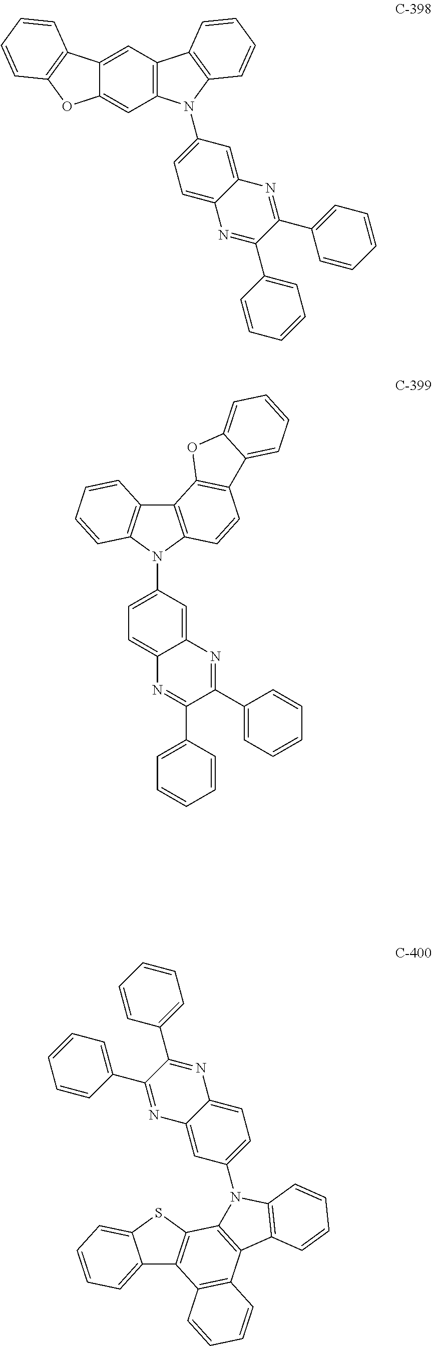 [SMILES: c1ccc(-c2nc3ccc(-n4c5ccccc5c5cc6c(cc54)oc4ccccc46)cc3nc2-c2ccccc2)cc1 ; c1ccc(-c2nc3ccc(-n4c5ccccc5c5c6oc7ccccc7c6ccc54)cc3nc2-c2ccccc2)cc1 ; c1ccc(-c2nc3ccc(-n4c5ccccc5c5c6ccccc6c6c7ccccc7sc6c54)cc3nc2-c2ccccc2)cc1]

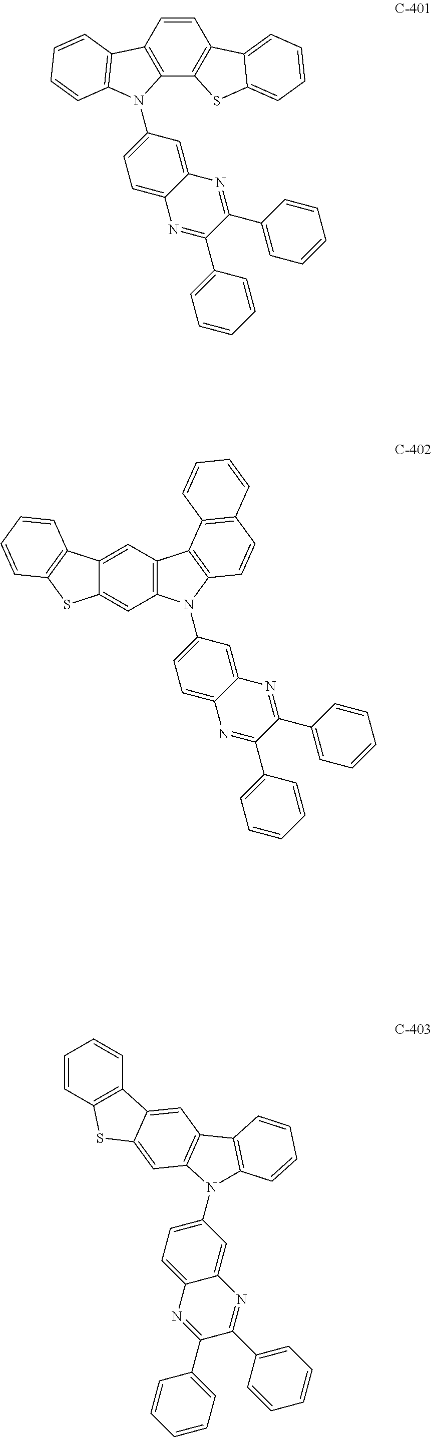 [SMILES: c1ccc(-c2nc3ccc(-n4c5ccccc5c5ccc6c7ccccc7sc6c54)cc3nc2-c2ccccc2)cc1 ; c1ccc(-c2nc3ccc(-n4c5cc6sc7ccccc7c6cc5c5c6ccccc6ccc54)cc3nc2-c2ccccc2)cc1 ; c1ccc(-c2nc3ccc(-n4c5ccccc5c5cc6c(cc54)sc4ccccc46)cc3nc2-c2ccccc2)cc1]

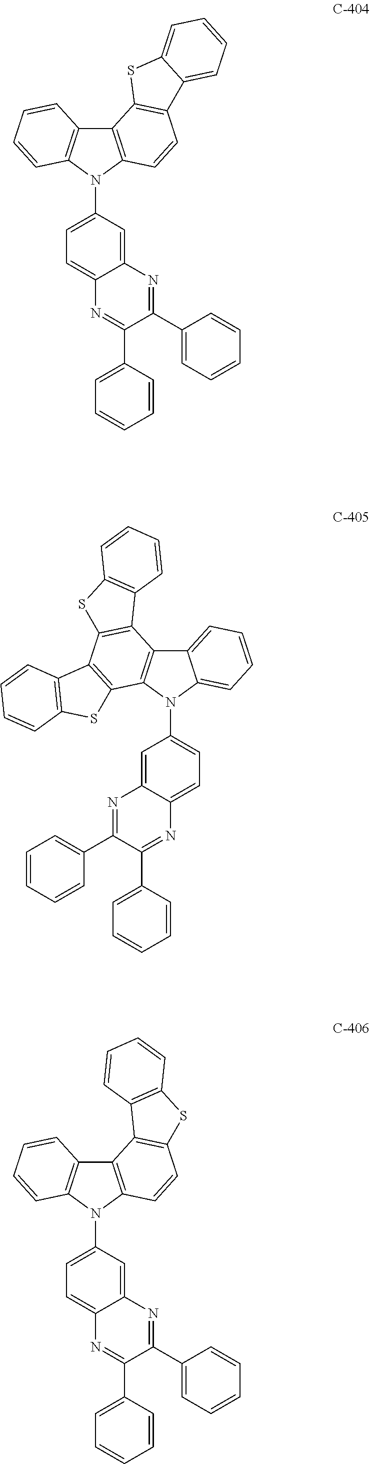 [SMILES: c1ccc(-c2nc3ccc(-n4c5ccccc5c5c6sc7ccccc7c6ccc54)cc3nc2-c2ccccc2)cc1 ; c1ccc(-c2nc3ccc(-n4c5ccccc5c5c6c7ccccc7sc6c6c7ccccc7sc6c54)cc3nc2-c2ccccc2)cc1 ; c1ccc(-c2nc3ccc(-n4c5ccccc5c5c6c(ccc54)sc4ccccc46)cc3nc2-c2ccccc2)cc1]

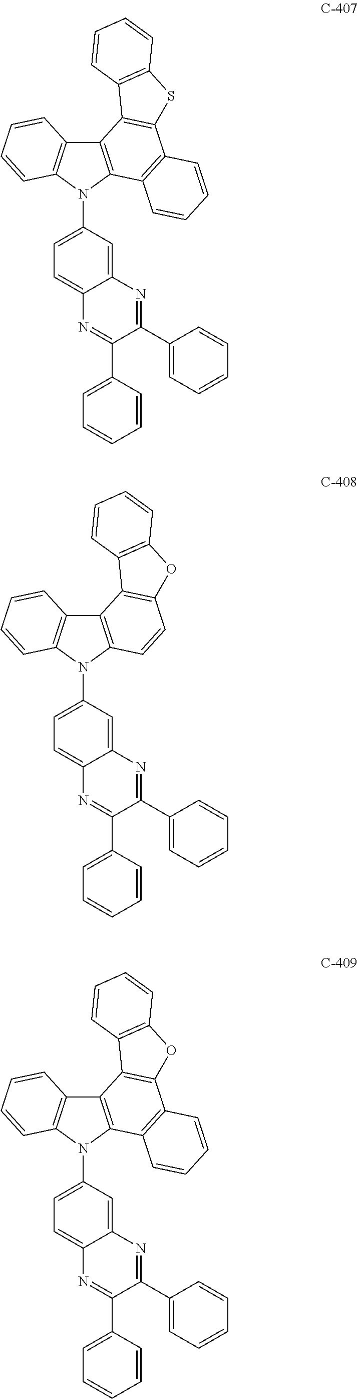 [SMILES: c1ccc(-c2nc3ccc(-n4c5ccccc5c5c6c7ccccc7sc6c6ccccc6c54)cc3nc2-c2ccccc2)cc1 ; c1ccc(-c2nc3ccc(-n4c5ccccc5c5c6c(ccc54)oc4ccccc46)cc3nc2-c2ccccc2)cc1 ; c1ccc(-c2nc3ccc(-n4c5ccccc5c5c6c7ccccc7oc6c6ccccc6c54)cc3nc2-c2ccccc2)cc1]

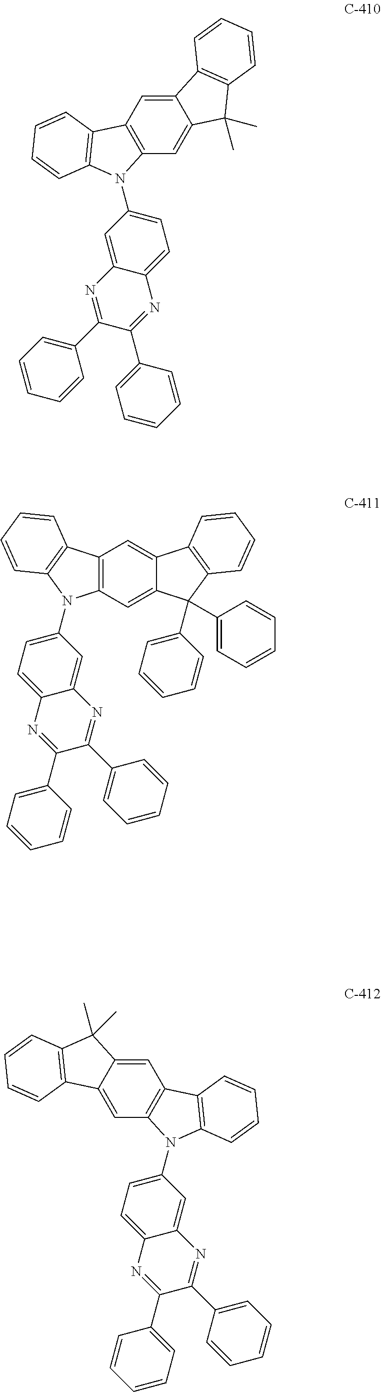

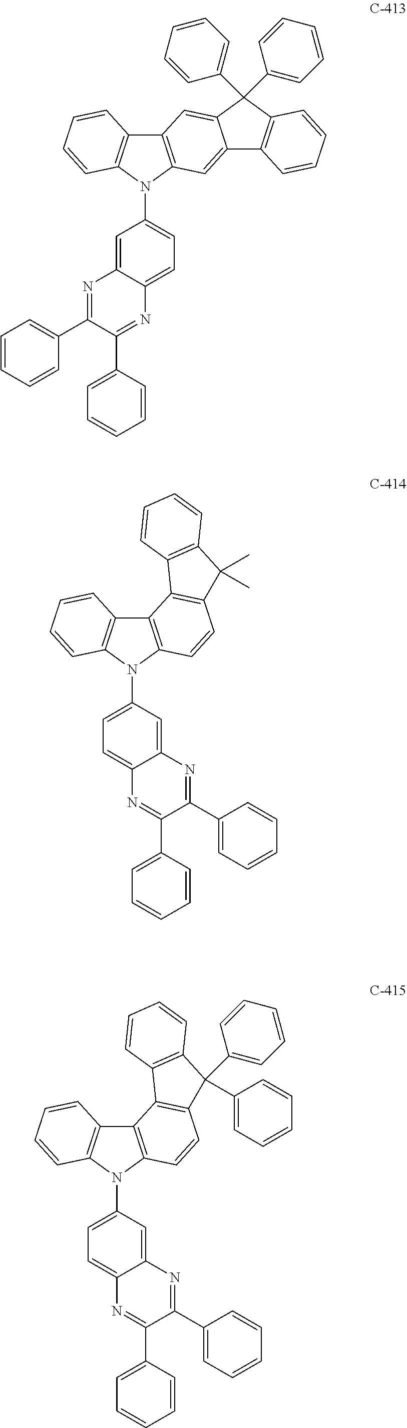 [SMILES: c1ccc(-c2nc3ccc(-n4c5ccccc5c5cc6c(cc54)-c4ccccc4C6(c4ccccc4)c4ccccc4)cc3nc2-c2ccccc2)cc1 ; CC1(C)c2ccccc2-c2c1ccc1c2c2ccccc2n1-c1ccc2nc(-c3ccccc3)c(-c3ccccc3)nc2c1 ; c1ccc(-c2nc3ccc(-n4c5ccccc5c5c6c(ccc54)C(c4ccccc4)(c4ccccc4)c4ccccc4-6)cc3nc2-c2ccccc2)cc1]

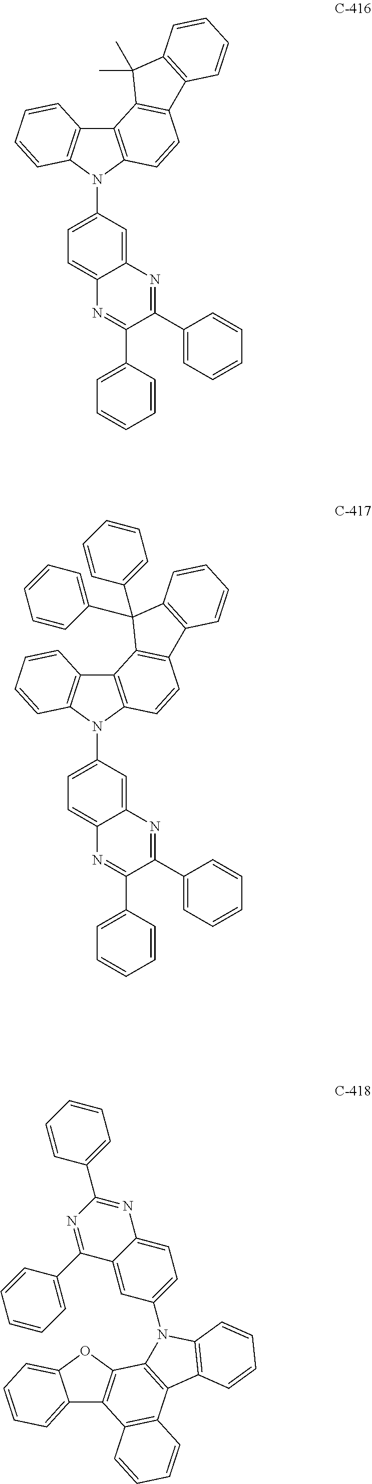 [SMILES: CC1(C)c2ccccc2-c2ccc3c(c21)c1ccccc1n3-c1ccc2nc(-c3ccccc3)c(-c3ccccc3)nc2c1 ; c1ccc(-c2nc3ccc(-n4c5ccccc5c5c6c(ccc54)-c4ccccc4C6(c4ccccc4)c4ccccc4)cc3nc2-c2ccccc2)cc1 ; c1ccc(-c2nc(-c3ccccc3)c3cc(-n4c5ccccc5c5c6ccccc6c6c7ccccc7oc6c54)ccc3n2)cc1]

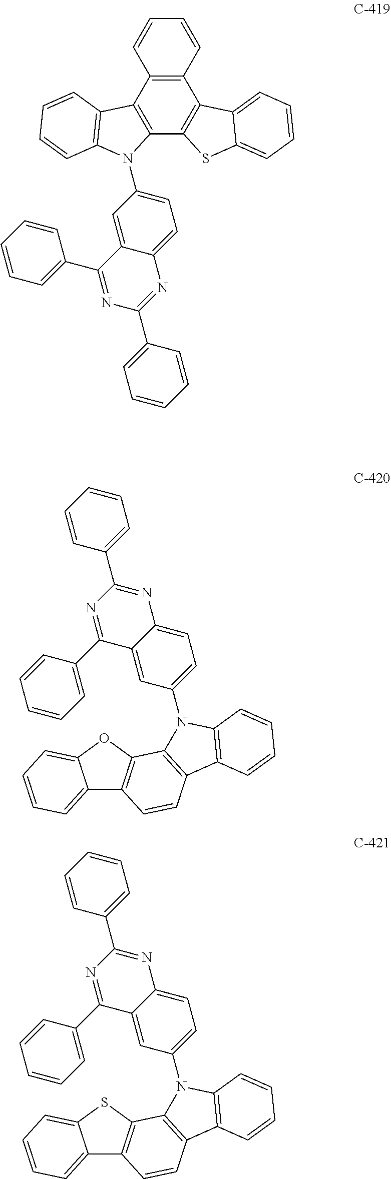

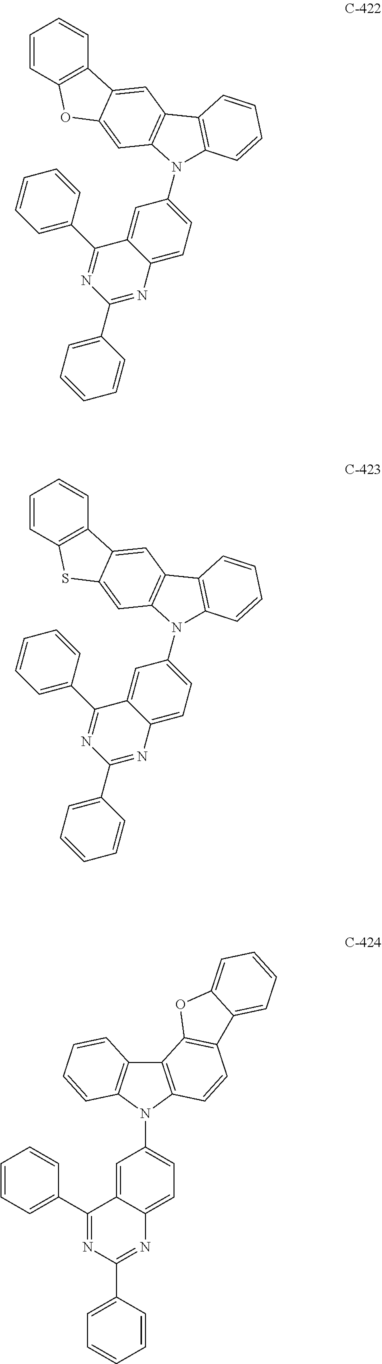 [SMILES: c1ccc(-c2nc(-c3ccccc3)c3cc(-n4c5ccccc5c5cc6c(cc54)oc4ccccc46)ccc3n2)cc1 ; c1ccc(-c2nc(-c3ccccc3)c3cc(-n4c5ccccc5c5cc6c(cc54)sc4ccccc46)ccc3n2)cc1 ; c1ccc(-c2nc(-c3ccccc3)c3cc(-n4c5ccccc5c5c6oc7ccccc7c6ccc54)ccc3n2)cc1]

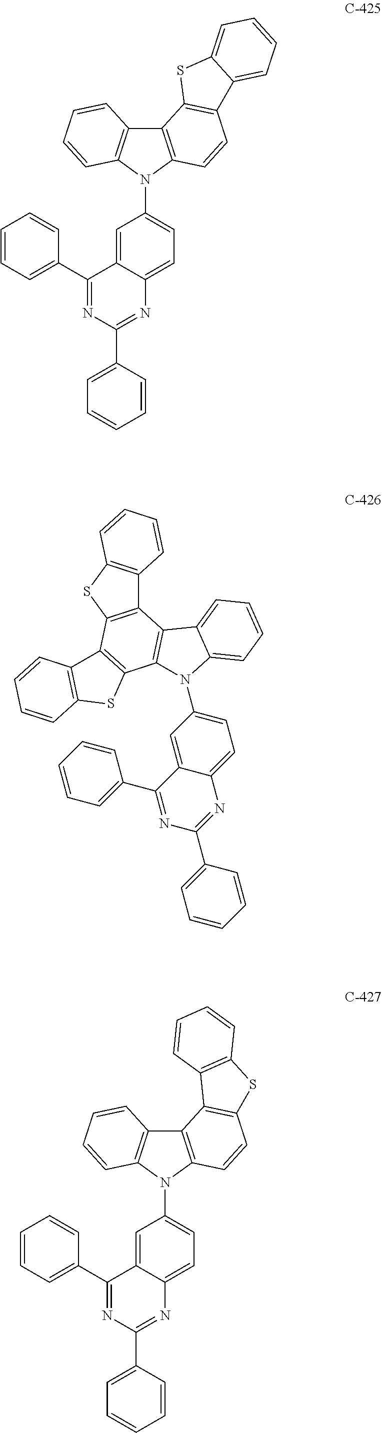 [SMILES: c1ccc(-c2nc(-c3ccccc3)c3cc(-n4c5ccccc5c5c6sc7ccccc7c6ccc54)ccc3n2)cc1 ; c1ccc(-c2nc(-c3ccccc3)c3cc(-n4c5ccccc5c5c6c7ccccc7sc6c6c7ccccc7sc6c54)ccc3n2)cc1 ; c1ccc(-c2nc(-c3ccccc3)c3cc(-n4c5ccccc5c5c6c(ccc54)sc4ccccc46)ccc3n2)cc1]

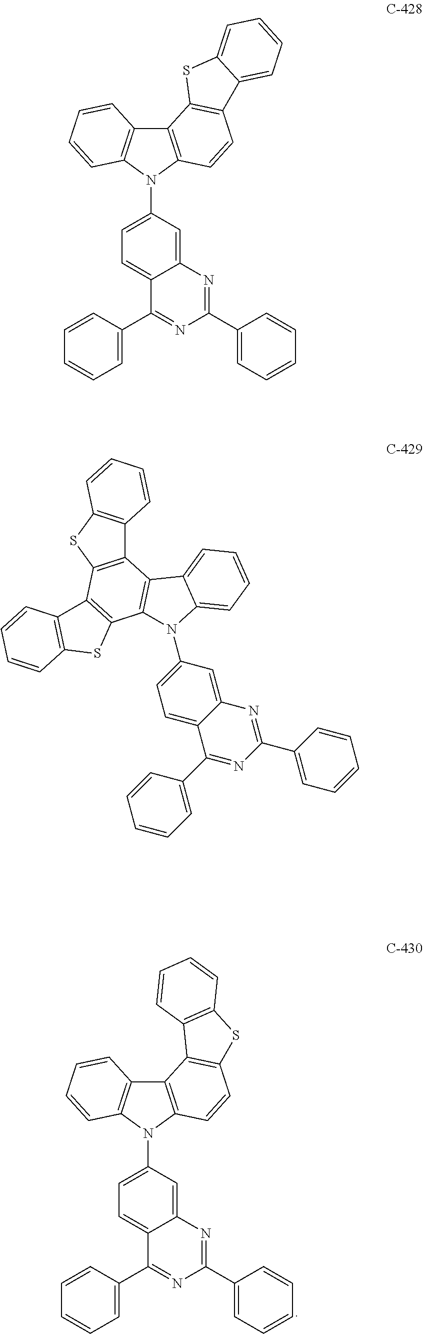 [SMILES: c1ccc(-c2nc(-c3ccccc3)c3ccc(-n4c5ccccc5c5c6sc7ccccc7c6ccc54)cc3n2)cc1 ; c1ccc(-c2nc(-c3ccccc3)c3ccc(-n4c5ccccc5c5c6c7ccccc7sc6c6c7ccccc7sc6c54)cc3n2)cc1 ; c1ccc(-c2nc(-c3ccccc3)c3ccc(-n4c5ccccc5c5c6c(ccc54)sc4ccccc46)cc3n2)cc1]

The compound of formula 1 according to the present disclosure can be prepared by a synthetic method known to a person skilled in the art. For example, it can be prepared according to the following reaction schemes.

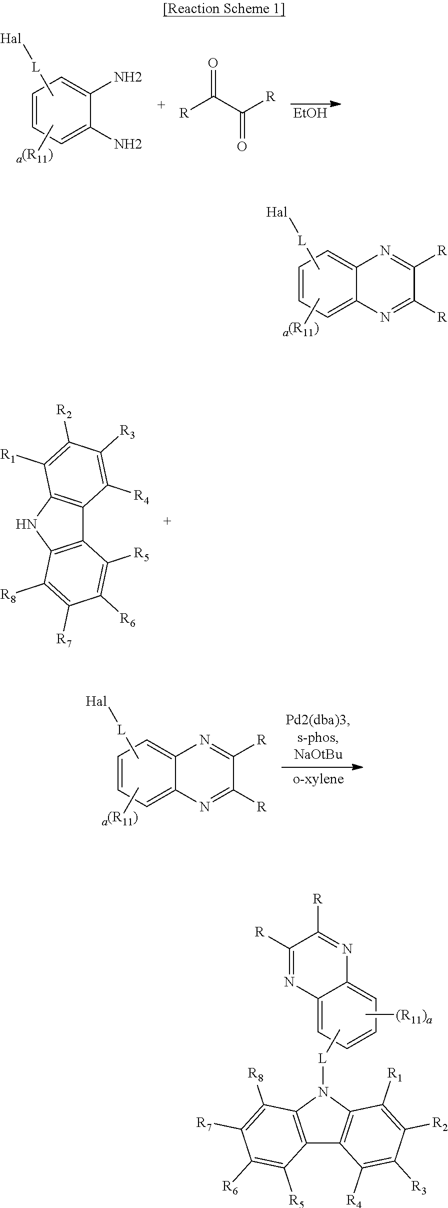

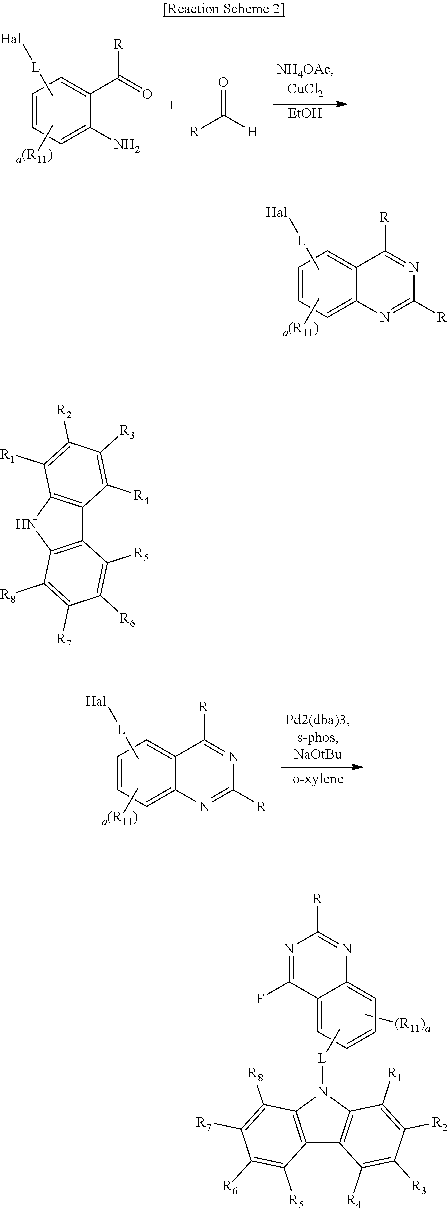

wherein R, R₁₁, L, R₁ to R₈, and a are as defined in formula 1, and Hal represents a halogen.

The present disclosure provides an organic electroluminescent material comprising the organic electroluminescent compound of formula 1, and an organic electroluminescent device comprising the material.

The above material can be comprised of the organic electroluminescent compound according to the present disclosure alone, or can further include conventional materials generally used in organic electroluminescent materials.

The organic electroluminescent compound of formula 1 of the present disclosure may be comprised in one or more layers of the light-emitting layer, the hole injection layer, the hole transport layer, the hole auxiliary layer, the light-emitting auxiliary layer, the electron transport layer, the electron buffer layer, the electron injection layer, the interlayer, the hole blocking layer, and the electron blocking layer; and preferably in one or more layers of the light-emitting layer and the electron buffer layer. Where used in the light-emitting layer, the organic electroluminescent compound of formula 1 of the present disclosure can be comprised as a host material. In addition, where used in the electron buffer layer, the organic electroluminescent compound of formula 1 of the present disclosure can be comprised as an electron buffer material. Preferably, the light-emitting layer can further comprise one or more dopants. If necessary, the organic electroluminescent compound of the present disclosure can be used as a co-host material. That is, the light-emitting layer can additionally comprise an organic electroluminescent compound other than the organic electroluminescent compound of formula 1 of the present disclosure (first host material) as a second host material. Herein, the weight ratio of the first host material to the second host material is in the range of 1:99 to 99:1.

The second host material can be any of the known hosts. The compound of the following formula 11 may be preferable.

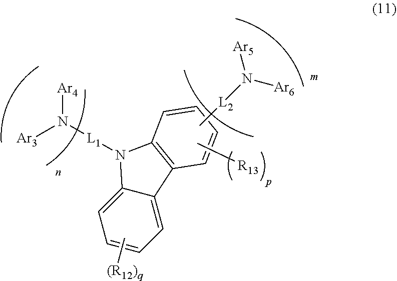

wherein
Ar₃ to Ar₆ each independently represent a substituted or unsubstituted (C6-C30)aryl;
L₁ represents a single bond, or a substituted or unsubstituted (C6-C30)aryl(ene);
L₂ represents a single bond, or a substituted or unsubstituted (C6-C30)arylene;
R₁₂ and R₁₃ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, or a substituted or unsubstituted (C1-C30)alkyl(C6-C30)arylamino; or may be linked to an adjacent substituent to form a ring;
m and n each independently represent an integer of 0 to 2, where at least one of m and n is 1 or more; and
p and q each independently represent an integer of 1 to 4, where p and q are an integer of 2 or more, each R₁ and each R₂ may be the same or different.

Formula 11 may be represented by the following formula 11-1 or 11-2.

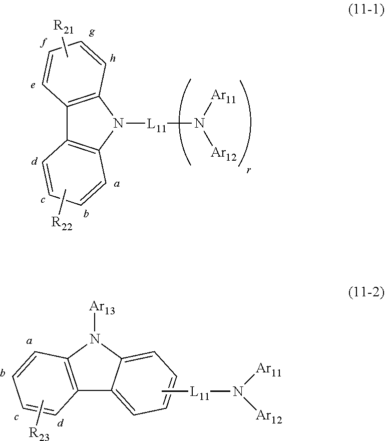

wherein
Ar₁₁ to Ar₁₃ each independently represent a substituted or unsubstituted (C6-C30)aryl;
L₁₁ represents a single bond, or a substituted or unsubstituted (C6-C30)arylene;
one or more positions of a and b, b and c, c and d, e and f, f and g, or g and h of formulas 11-1 and 11-2, and two * positions of the following formula 11-a, 11-b, or 11-c may be fused to each other to form a ring;

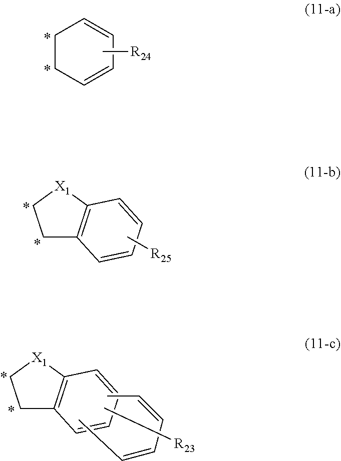

wherein
X₁ represents NR₃₁, O, S, or CR₃₂R₃₃;
R₃₁ to R₃₃ each independently represent a substituted or unsubstituted (C1-C30)alkyl, a substituted or unsubstituted (C6-C30)aryl, or a substituted or unsubstituted (3- to 30-membered)heteroaryl;
R₂₁ to R₂₆ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, or a substituted or unsubstituted (C1-C30)alkyl(C6-C30)arylamino; and
r represents 1 or 2.

Specifically, the examples of the second host material are as follows, but are not limited thereto.

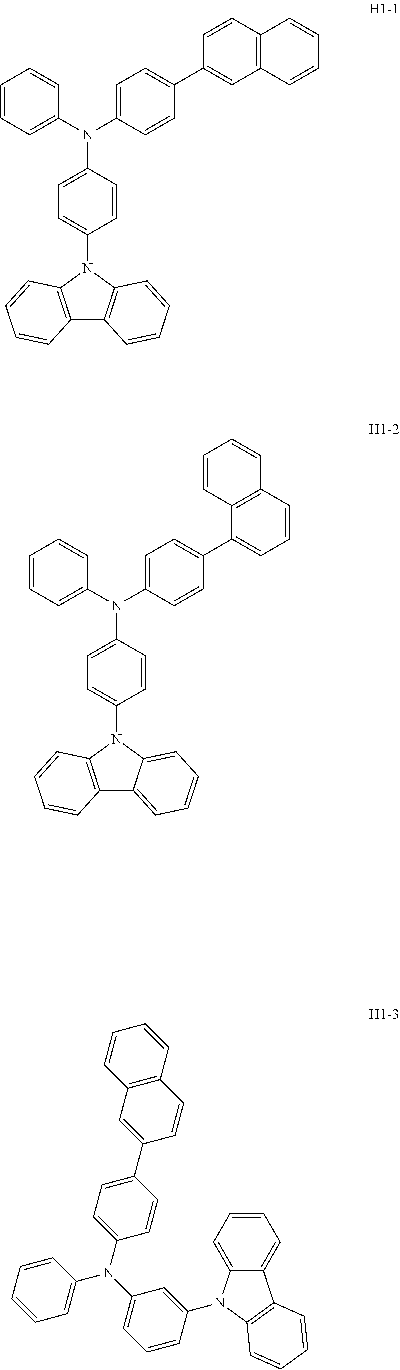 [SMILES: c1ccc(N(c2ccc(-c3ccc4ccccc4c3)cc2)c2ccc(-n3c4ccccc4c4ccccc43)cc2)cc1 ; c1ccc(N(c2ccc(-c3cccc4ccccc34)cc2)c2ccc(-n3c4ccccc4c4ccccc43)cc2)cc1 ; c1ccc(N(c2ccc(-c3ccc4ccccc4c3)cc2)c2cccc(-n3c4ccccc4c4ccccc43)c2)cc1]

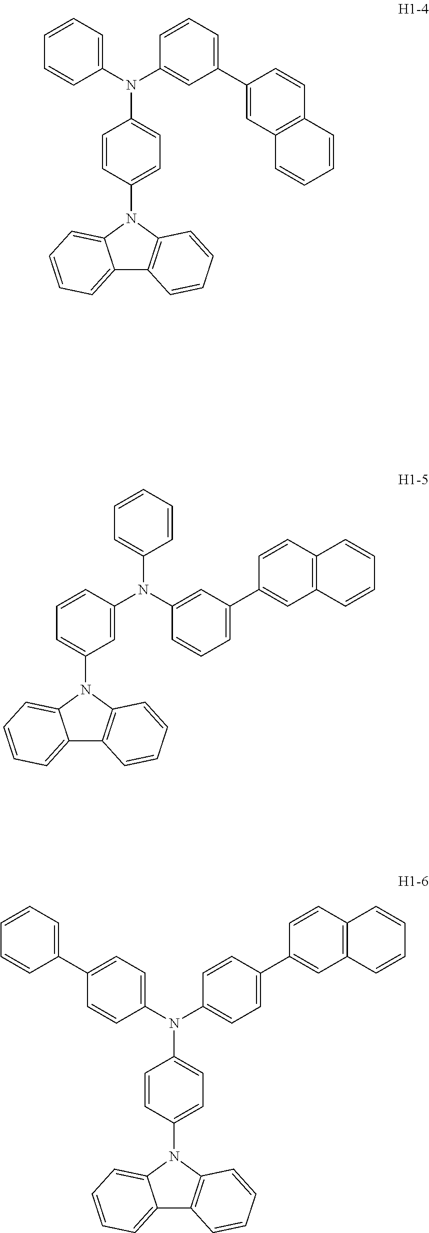 [SMILES: c1ccc(N(c2ccc(-n3c4ccccc4c4ccccc43)cc2)c2cccc(-c3ccc4ccccc4c3)c2)cc1 ; c1ccc(N(c2cccc(-c3ccc4ccccc4c3)c2)c2cccc(-n3c4ccccc4c4ccccc43)c2)cc1 ; c1ccc(-c2ccc(N(c3ccc(-c4ccc5ccccc5c4)cc3)c3ccc(-n4c5ccccc5c5ccccc54)cc3)cc2)cc1]

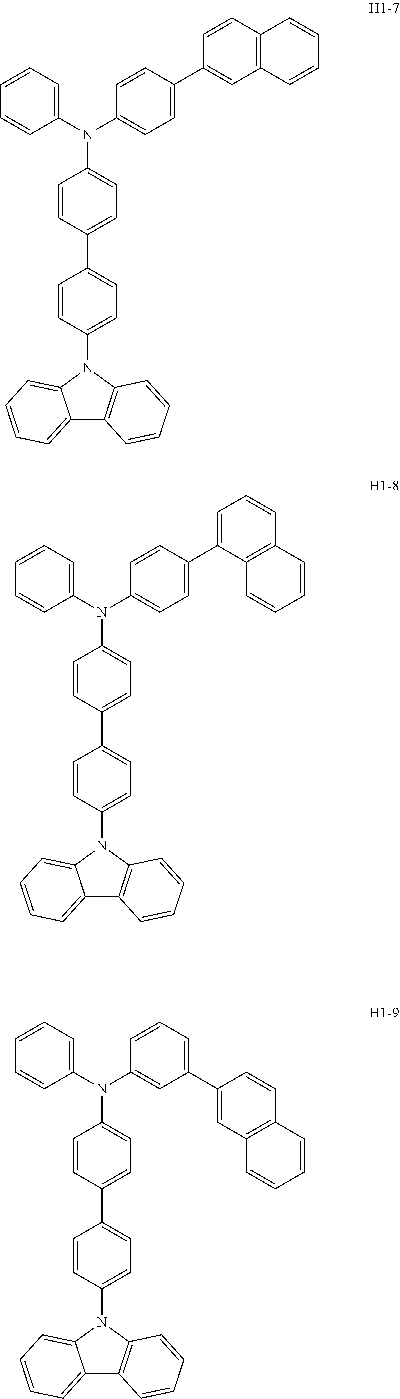 [SMILES: c1ccc(N(c2ccc(-c3ccc(-n4c5ccccc5c5ccccc54)cc3)cc2)c2ccc(-c3ccc4ccccc4c3)cc2)cc1 ; c1ccc(N(c2ccc(-c3ccc(-n4c5ccccc5c5ccccc54)cc3)cc2)c2ccc(-c3cccc4ccccc34)cc2)cc1 ; c1ccc(N(c2ccc(-c3ccc(-n4c5ccccc5c5ccccc54)cc3)cc2)c2cccc(-c3ccc4ccccc4c3)c2)cc1]

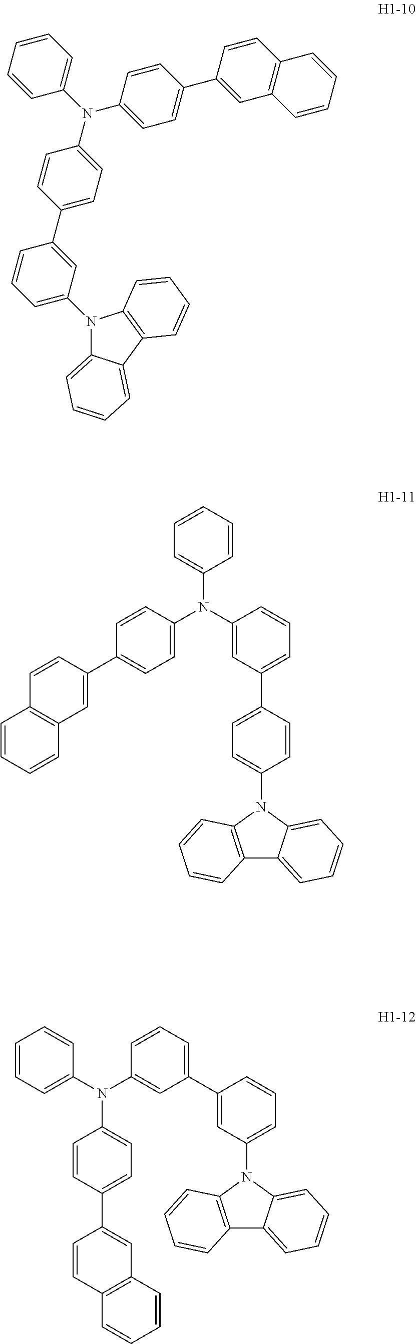 [SMILES: c1ccc(N(c2ccc(-c3cccc(-n4c5ccccc5c5ccccc54)c3)cc2)c2ccc(-c3ccc4ccccc4c3)cc2)cc1 ; c1ccc(N(c2ccc(-c3ccc4ccccc4c3)cc2)c2cccc(-c3ccc(-n4c5ccccc5c5ccccc54)cc3)c2)cc1 ; c1ccc(N(c2ccc(-c3ccc4ccccc4c3)cc2)c2cccc(-c3cccc(-n4c5ccccc5c5ccccc54)c3)c2)cc1]

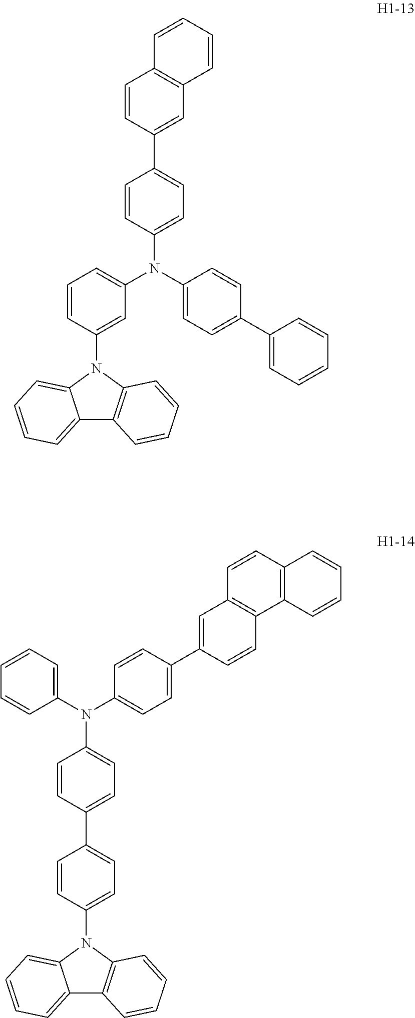 [SMILES: c1ccc(-c2ccc(N(c3ccc(-c4ccc5ccccc5c4)cc3)c3cccc(-n4c5ccccc5c5ccccc54)c3)cc2)cc1 ; c1ccc(N(c2ccc(-c3ccc(-n4c5ccccc5c5ccccc54)cc3)cc2)c2ccc(-c3ccc4c(ccc5ccccc54)c3)cc2)cc1]

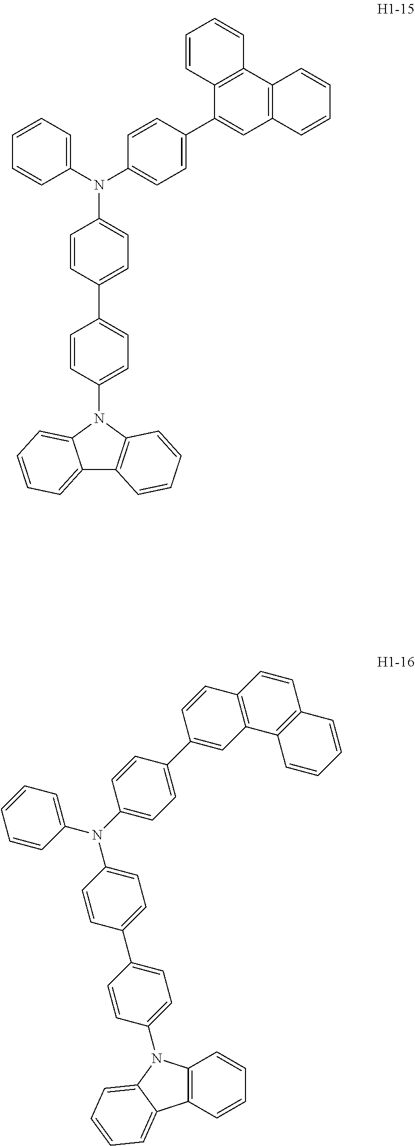 [SMILES: c1ccc(N(c2ccc(-c3ccc(-n4c5ccccc5c5ccccc54)cc3)cc2)c2ccc(-c3cc4ccccc4c4ccccc34)cc2)cc1 ; c1ccc(N(c2ccc(-c3ccc(-n4c5ccccc5c5ccccc54)cc3)cc2)c2ccc(-c3ccc4ccc5ccccc5c4c3)cc2)cc1]

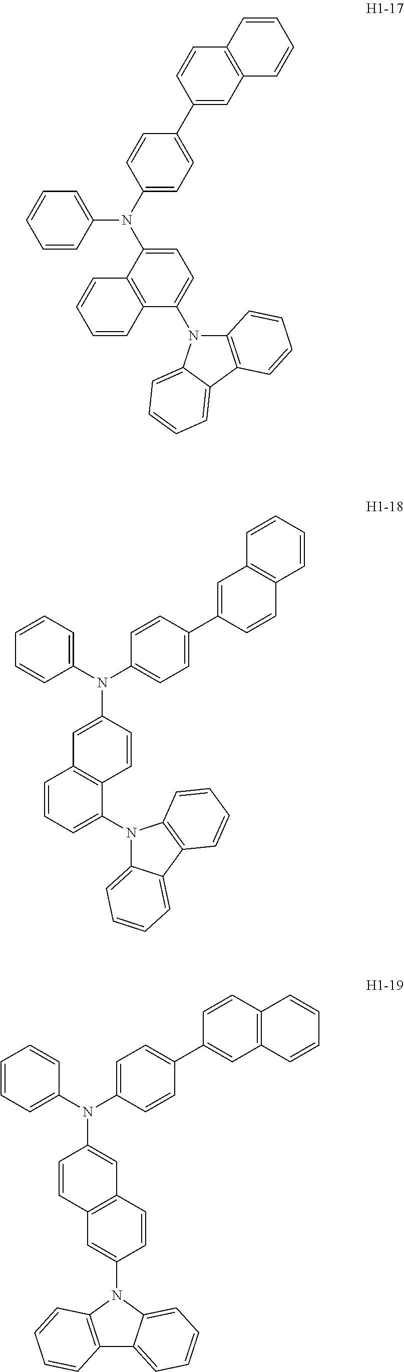 [SMILES: c1ccc(N(c2ccc(-c3ccc4ccccc4c3)cc2)c2ccc(-n3c4ccccc4c4ccccc43)c3ccccc23)cc1 ; c1ccc(N(c2ccc(-c3ccc4ccccc4c3)cc2)c2ccc3c(-n4c5ccccc5c5ccccc54)cccc3c2)cc1 ; c1ccc(N(c2ccc(-c3ccc4ccccc4c3)cc2)c2ccc3cc(-n4c5ccccc5c5ccccc54)ccc3c2)cc1]

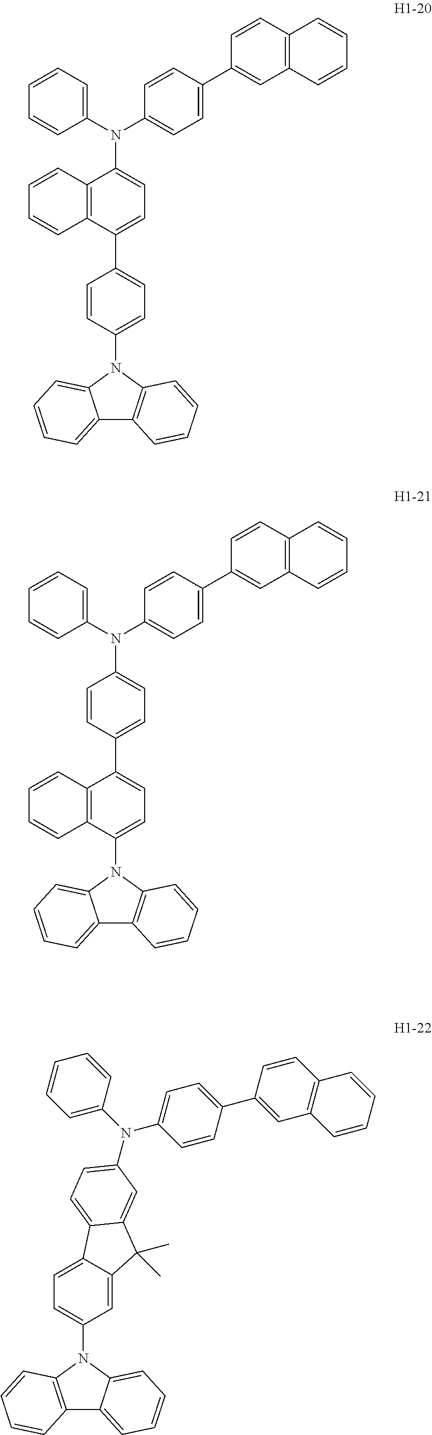 [SMILES: c1ccc(N(c2ccc(-c3ccc4ccccc4c3)cc2)c2ccc(-c3ccc(-n4c5ccccc5c5ccccc54)cc3)c3ccccc23)cc1 ; c1ccc(N(c2ccc(-c3ccc4ccccc4c3)cc2)c2ccc(-c3ccc(-n4c5ccccc5c5ccccc54)c4ccccc34)cc2)cc1 ; CC1(C)c2cc(N(c3ccccc3)c3ccc(-c4ccc5ccccc5c4)cc3)ccc2-c2ccc(-n3c4ccccc4c4ccccc43)cc21]

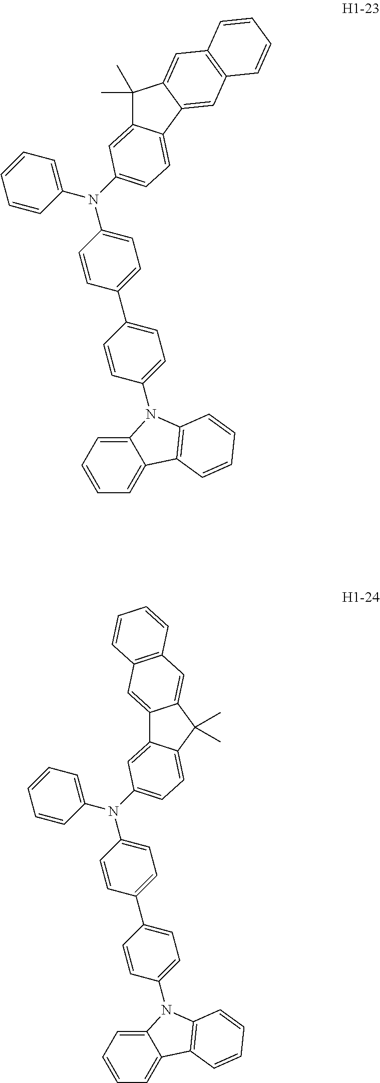 [SMILES: CC1(C)c2cc(N(c3ccccc3)c3ccc(-c4ccc(-n5c6ccccc6c6ccccc65)cc4)cc3)ccc2-c2cc3ccccc3cc21 ; CC1(C)c2ccc(N(c3ccccc3)c3ccc(-c4ccc(-n5c6ccccc6c6ccccc65)cc4)cc3)cc2-c2cc3ccccc3cc21]

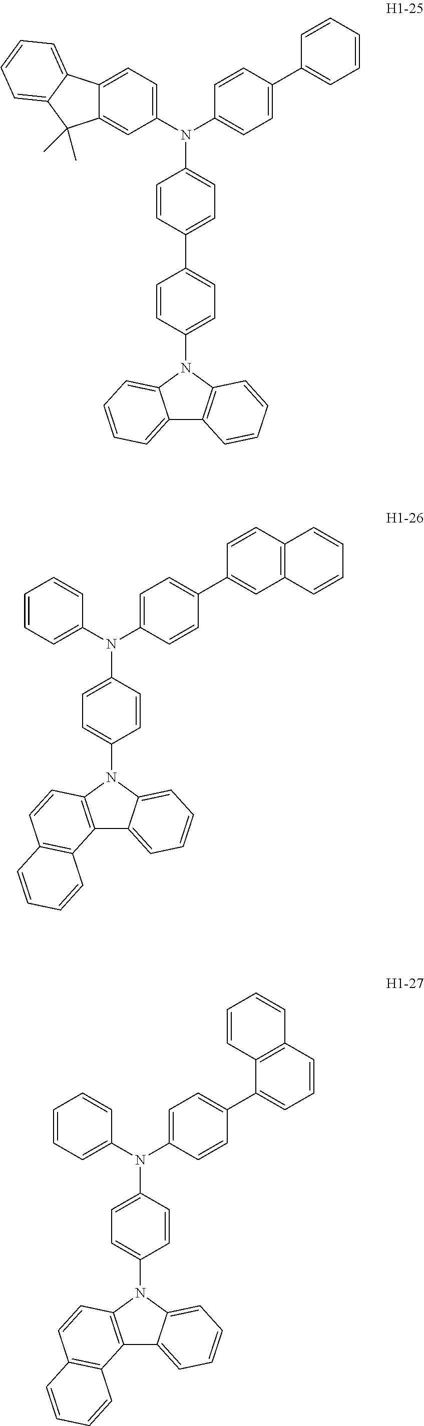 [SMILES: CC1(C)c2ccccc2-c2ccc(N(c3ccc(-c4ccccc4)cc3)c3ccc(-c4ccc(-n5c6ccccc6c6ccccc65)cc4)cc3)cc21 ; c1ccc(N(c2ccc(-c3ccc4ccccc4c3)cc2)c2ccc(-n3c4ccccc4c4c5ccccc5ccc43)cc2)cc1 ; c1ccc(N(c2ccc(-c3cccc4ccccc34)cc2)c2ccc(-n3c4ccccc4c4c5ccccc5ccc43)cc2)cc1]

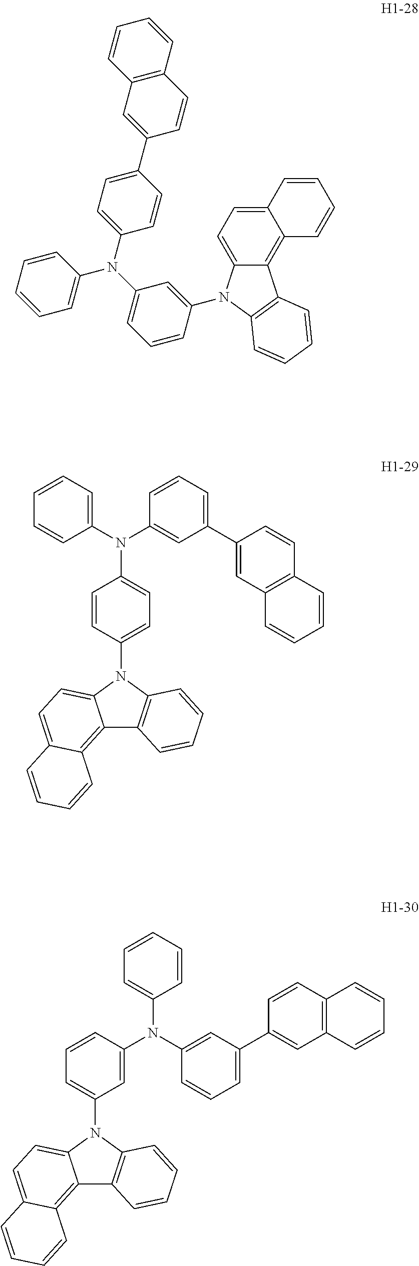 [SMILES: c1ccc(N(c2ccc(-c3ccc4ccccc4c3)cc2)c2cccc(-n3c4ccccc4c4c5ccccc5ccc43)c2)cc1 ; c1ccc(N(c2ccc(-n3c4ccccc4c4c5ccccc5ccc43)cc2)c2cccc(-c3ccc4ccccc4c3)c2)cc1 ; c1ccc(N(c2cccc(-c3ccc4ccccc4c3)c2)c2cccc(-n3c4ccccc4c4c5ccccc5ccc43)c2)cc1]

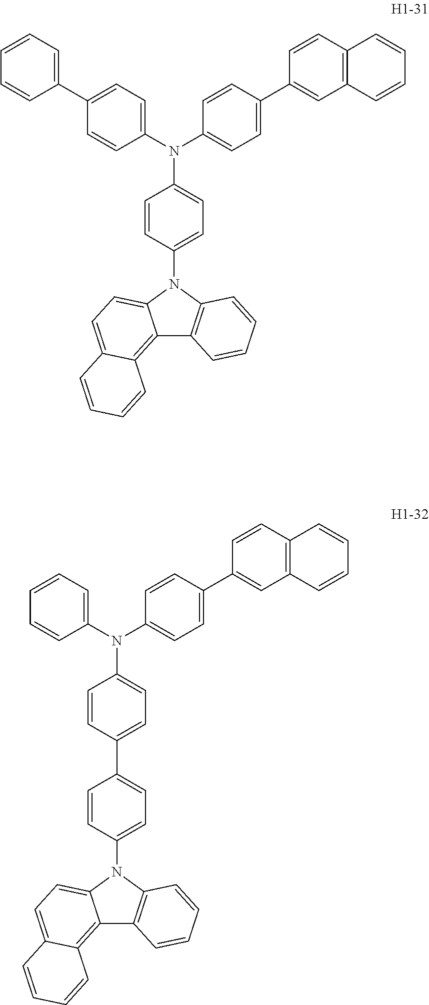 [SMILES: c1ccc(-c2ccc(N(c3ccc(-c4ccc5ccccc5c4)cc3)c3ccc(-n4c5ccccc5c5c6ccccc6ccc54)cc3)cc2)cc1 ; c1ccc(N(c2ccc(-c3ccc(-n4c5ccccc5c5c6ccccc6ccc54)cc3)cc2)c2ccc(-c3ccc4ccccc4c3)cc2)cc1]

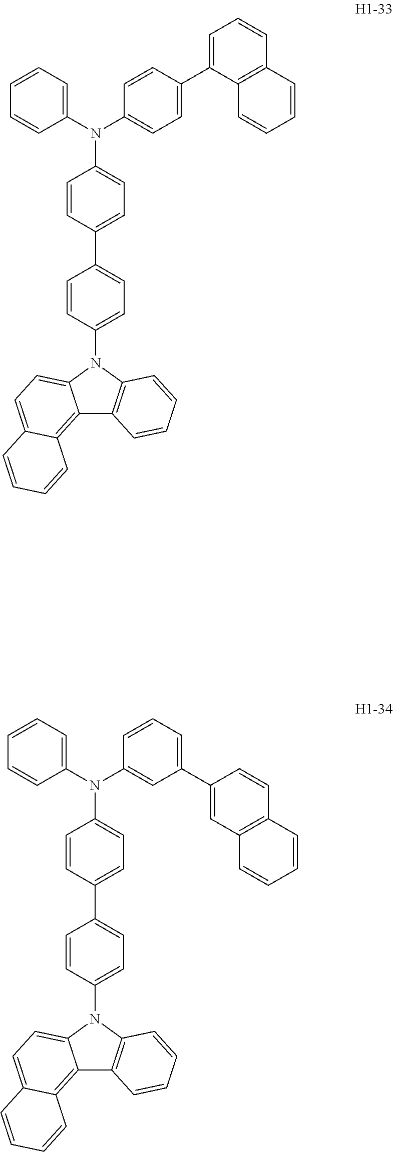 [SMILES: c1ccc(N(c2ccc(-c3ccc(-n4c5ccccc5c5c6ccccc6ccc54)cc3)cc2)c2ccc(-c3cccc4ccccc34)cc2)cc1 ; c1ccc(N(c2ccc(-c3ccc(-n4c5ccccc5c5c6ccccc6ccc54)cc3)cc2)c2cccc(-c3ccc4ccccc4c3)c2)cc1]

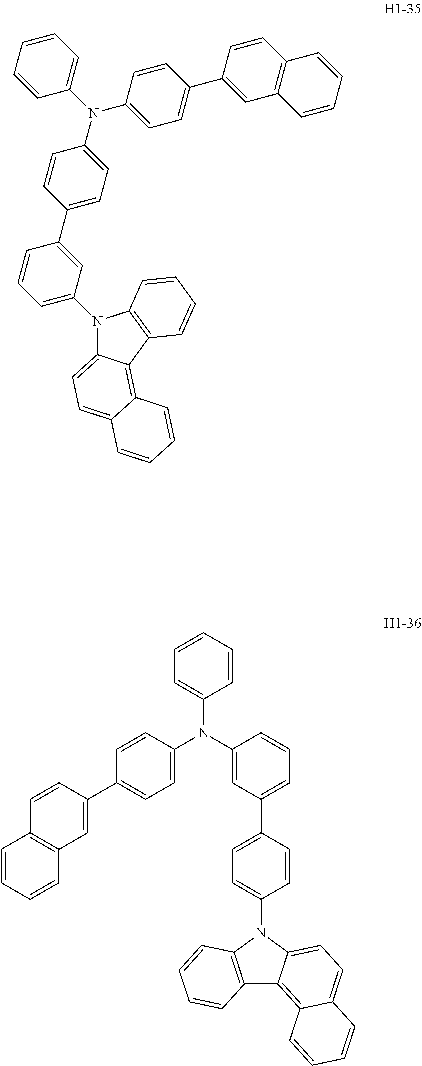 [SMILES: c1ccc(N(c2ccc(-c3cccc(-n4c5ccccc5c5c6ccccc6ccc54)c3)cc2)c2ccc(-c3ccc4ccccc4c3)cc2)cc1 ; c1ccc(N(c2ccc(-c3ccc4ccccc4c3)cc2)c2cccc(-c3ccc(-n4c5ccccc5c5c6ccccc6ccc54)cc3)c2)cc1]

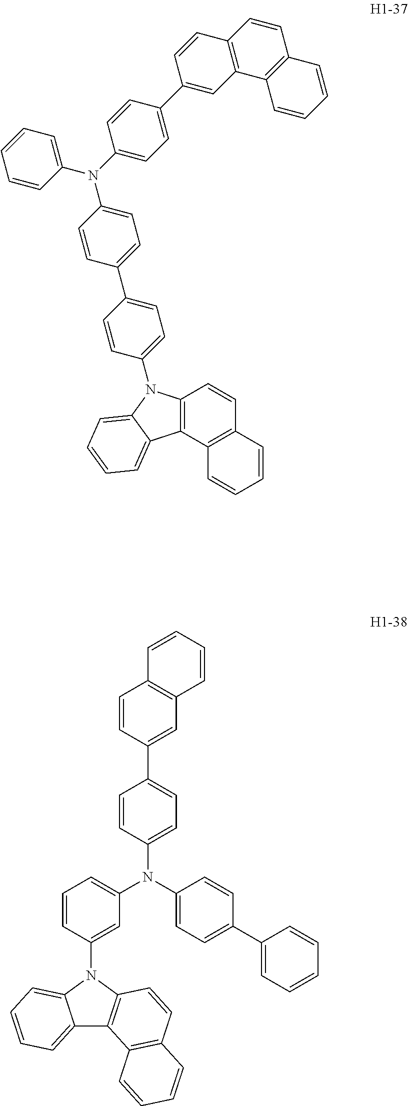 [SMILES: c1ccc(N(c2ccc(-c3ccc(-n4c5ccccc5c5c6ccccc6ccc54)cc3)cc2)c2ccc(-c3ccc4ccc5ccccc5c4c3)cc2)cc1 ; c1ccc(-c2ccc(N(c3ccc(-c4ccc5ccccc5c4)cc3)c3cccc(-n4c5ccccc5c5c6ccccc6ccc54)c3)cc2)cc1]

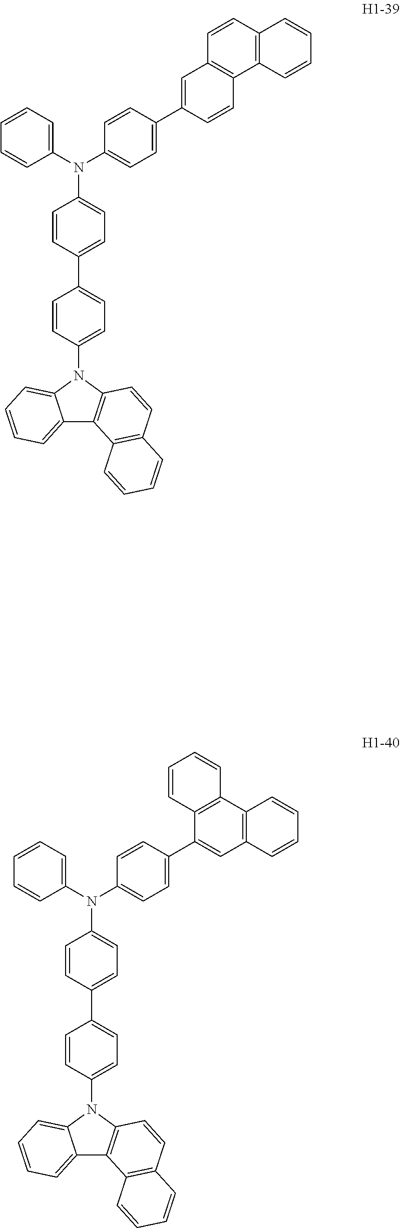 [SMILES: c1ccc(N(c2ccc(-c3ccc(-n4c5ccccc5c5c6ccccc6ccc54)cc3)cc2)c2ccc(-c3ccc4c(ccc5ccccc54)c3)cc2)cc1 ; c1ccc(N(c2ccc(-c3ccc(-n4c5ccccc5c5c6ccccc6ccc54)cc3)cc2)c2ccc(-c3cc4ccccc4c4ccccc34)cc2)cc1]

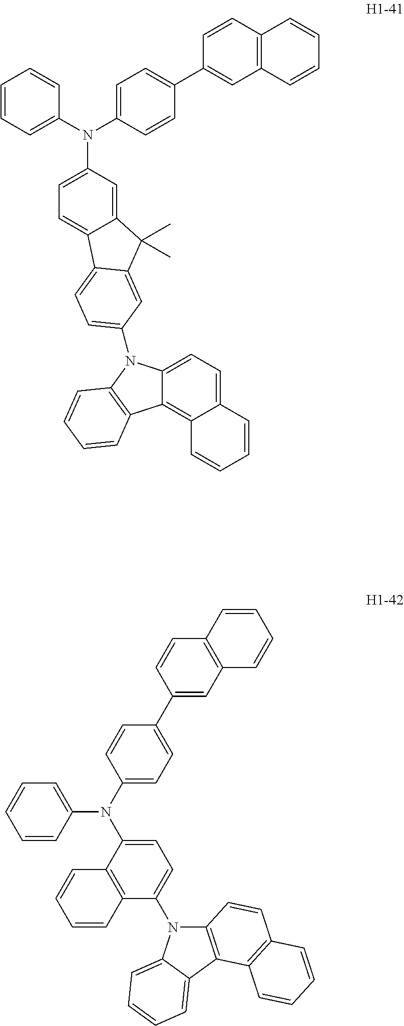 [SMILES: CC1(C)c2cc(N(c3ccccc3)c3ccc(-c4ccc5ccccc5c4)cc3)ccc2-c2ccc(-n3c4ccccc4c4c5ccccc5ccc43)cc21 ; c1ccc(N(c2ccc(-c3ccc4ccccc4c3)cc2)c2ccc(-n3c4ccccc4c4c5ccccc5ccc43)c3ccccc23)cc1]

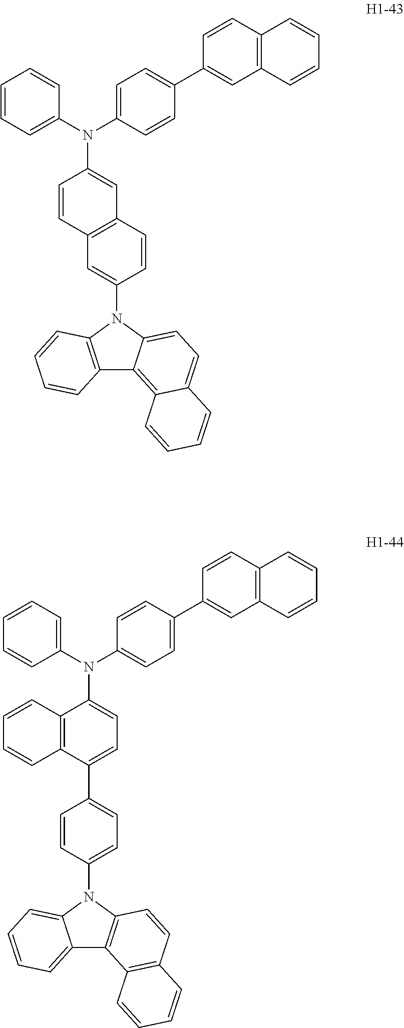 [SMILES: c1ccc(N(c2ccc(-c3ccc4ccccc4c3)cc2)c2ccc3cc(-n4c5ccccc5c5c6ccccc6ccc54)ccc3c2)cc1 ; c1ccc(N(c2ccc(-c3ccc4ccccc4c3)cc2)c2ccc(-c3ccc(-n4c5ccccc5c5c6ccccc6ccc54)cc3)c3ccccc23)cc1]

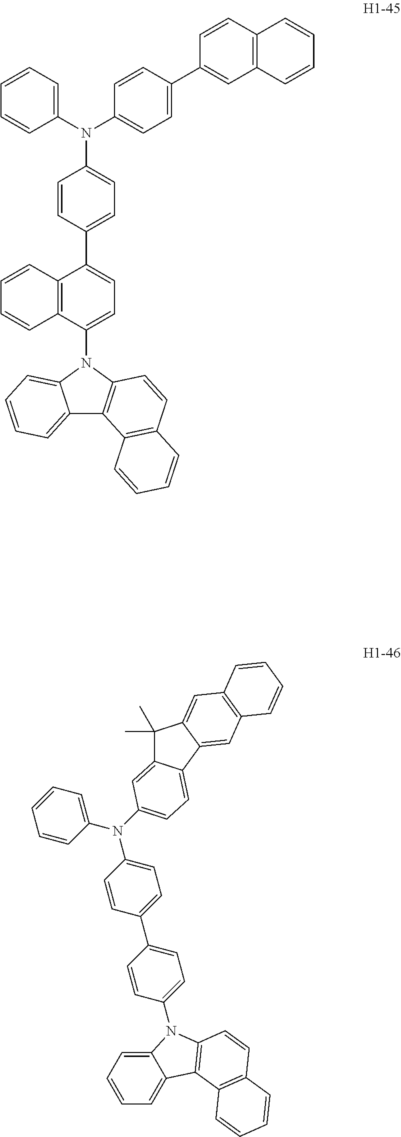 [SMILES: c1ccc(N(c2ccc(-c3ccc4ccccc4c3)cc2)c2ccc(-c3ccc(-n4c5ccccc5c5c6ccccc6ccc54)c4ccccc34)cc2)cc1 ; CC1(C)c2cc(N(c3ccccc3)c3ccc(-c4ccc(-n5c6ccccc6c6c7ccccc7ccc65)cc4)cc3)ccc2-c2cc3ccccc3cc21]

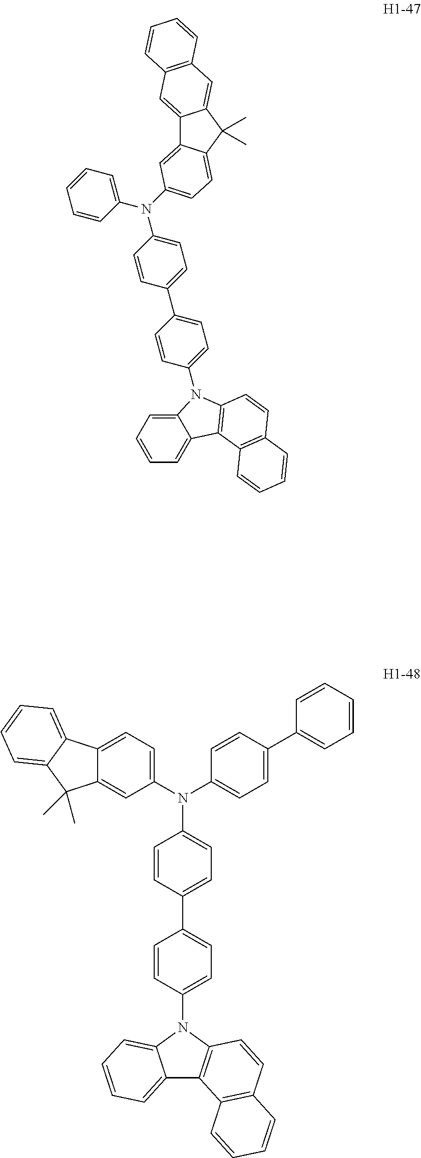 [SMILES: CC1(C)c2ccc(N(c3ccccc3)c3ccc(-c4ccc(-n5c6ccccc6c6c7ccccc7ccc65)cc4)cc3)cc2-c2cc3ccccc3cc21 ; CC1(C)c2ccccc2-c2ccc(N(c3ccc(-c4ccccc4)cc3)c3ccc(-c4ccc(-n5c6ccccc6c6c7ccccc7ccc65)cc4)cc3)cc21]

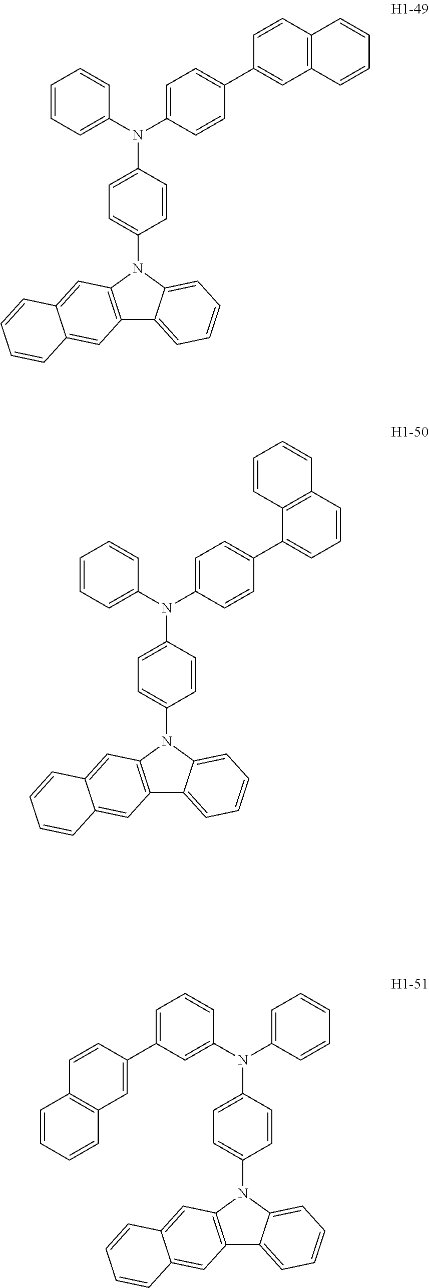 [SMILES: c1ccc(N(c2ccc(-c3ccc4ccccc4c3)cc2)c2ccc(-n3c4ccccc4c4cc5ccccc5cc43)cc2)cc1 ; c1ccc(N(c2ccc(-c3cccc4ccccc34)cc2)c2ccc(-n3c4ccccc4c4cc5ccccc5cc43)cc2)cc1 ; c1ccc(N(c2ccc(-n3c4ccccc4c4cc5ccccc5cc43)cc2)c2cccc(-c3ccc4ccccc4c3)c2)cc1]

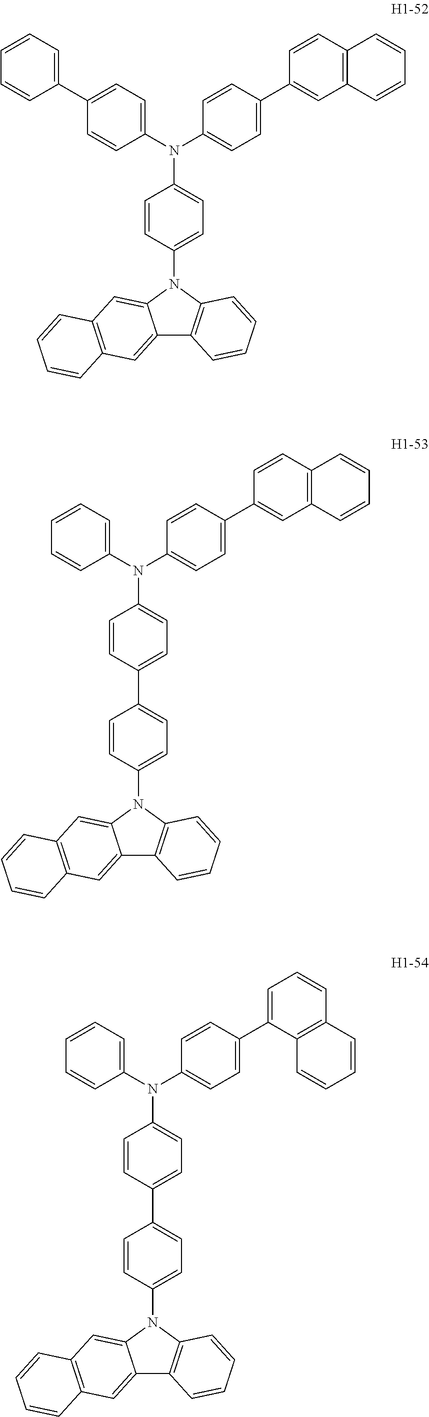 [SMILES: c1ccc(-c2ccc(N(c3ccc(-c4ccc5ccccc5c4)cc3)c3ccc(-n4c5ccccc5c5cc6ccccc6cc54)cc3)cc2)cc1 ; c1ccc(N(c2ccc(-c3ccc(-n4c5ccccc5c5cc6ccccc6cc54)cc3)cc2)c2ccc(-c3ccc4ccccc4c3)cc2)cc1 ; c1ccc(N(c2ccc(-c3ccc(-n4c5ccccc5c5cc6ccccc6cc54)cc3)cc2)c2ccc(-c3cccc4ccccc34)cc2)cc1]

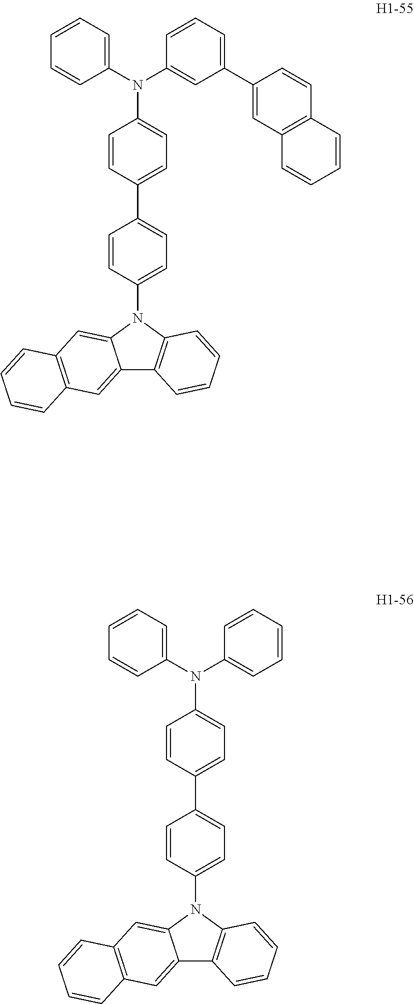 [SMILES: c1ccc(N(c2ccc(-c3ccc(-n4c5ccccc5c5cc6ccccc6cc54)cc3)cc2)c2cccc(-c3ccc4ccccc4c3)c2)cc1 ; c1ccc(N(c2ccccc2)c2ccc(-c3ccc(-n4c5ccccc5c5cc6ccccc6cc54)cc3)cc2)cc1]

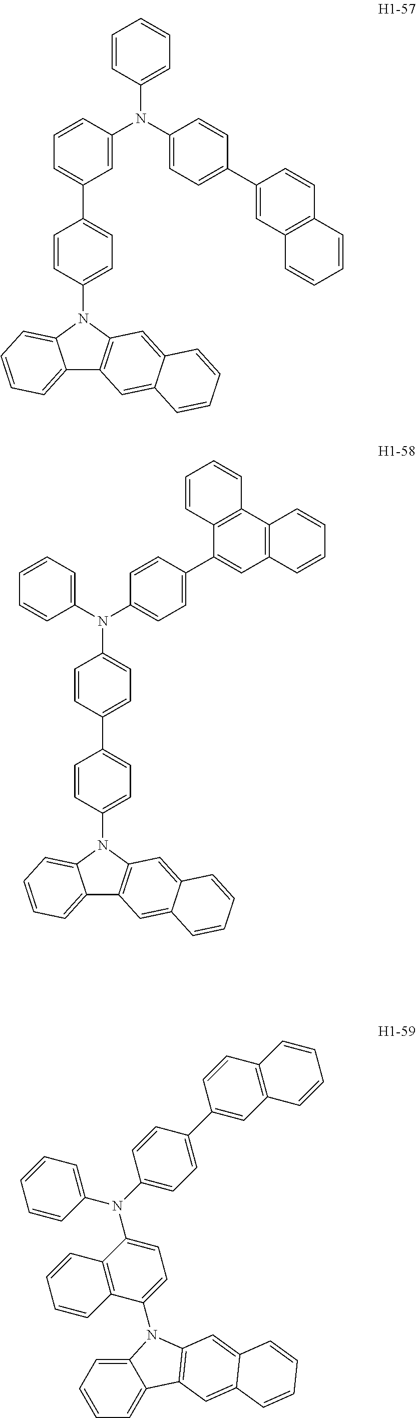 [SMILES: c1ccc(N(c2ccc(-c3ccc4ccccc4c3)cc2)c2cccc(-c3ccc(-n4c5ccccc5c5cc6ccccc6cc54)cc3)c2)cc1 ; c1ccc(N(c2ccc(-c3ccc(-n4c5ccccc5c5cc6ccccc6cc54)cc3)cc2)c2ccc(-c3cc4ccccc4c4ccccc34)cc2)cc1 ; c1ccc(N(c2ccc(-c3ccc4ccccc4c3)cc2)c2ccc(-n3c4ccccc4c4cc5ccccc5cc43)c3ccccc23)cc1]

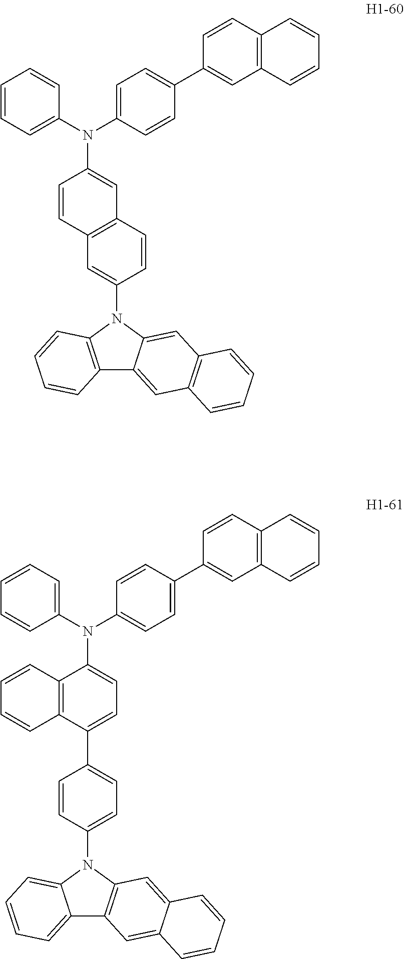 [SMILES: c1ccc(N(c2ccc(-c3ccc4ccccc4c3)cc2)c2ccc3cc(-n4c5ccccc5c5cc6ccccc6cc54)ccc3c2)cc1 ; c1ccc(N(c2ccc(-c3ccc4ccccc4c3)cc2)c2ccc(-c3ccc(-n4c5ccccc5c5cc6ccccc6cc54)cc3)c3ccccc23)cc1]

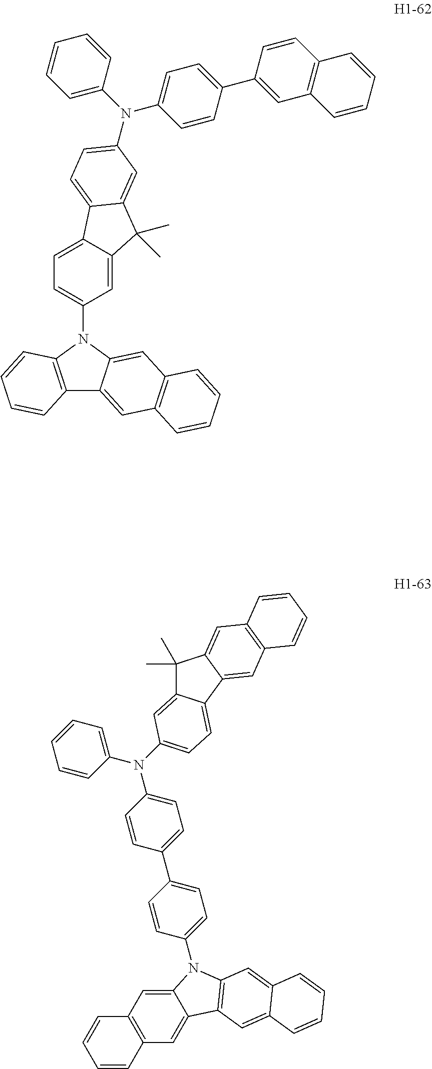 [SMILES: CC1(C)c2cc(N(c3ccccc3)c3ccc(-c4ccc5ccccc5c4)cc3)ccc2-c2ccc(-n3c4ccccc4c4cc5ccccc5cc43)cc21 ; CC1(C)c2cc(N(c3ccccc3)c3ccc(-c4ccc(-n5c6cc7ccccc7cc6c6cc7ccccc7cc65)cc4)cc3)ccc2-c2cc3ccccc3cc21]

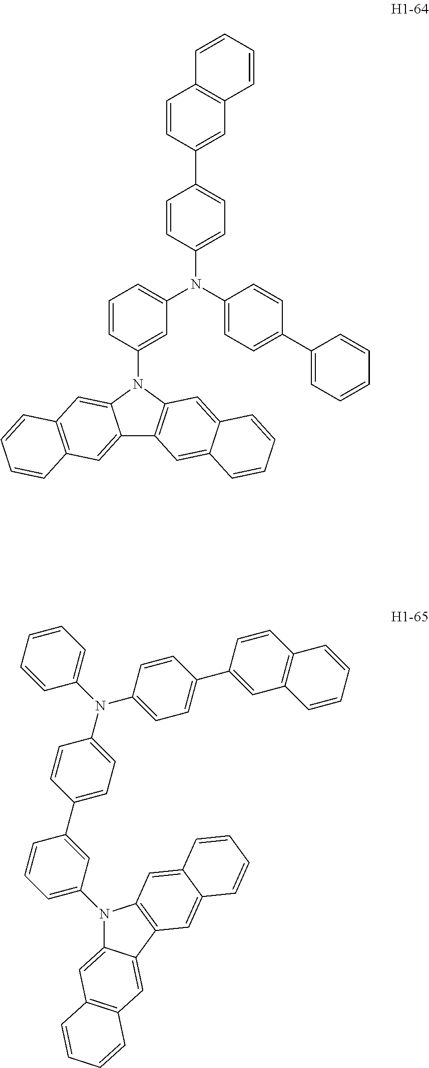 [SMILES: c1ccc(-c2ccc(N(c3ccc(-c4ccc5ccccc5c4)cc3)c3cccc(-n4c5cc6ccccc6cc5c5cc6ccccc6cc54)c3)cc2)cc1 ; c1ccc(N(c2ccc(-c3cccc(-n4c5cc6ccccc6cc5c5cc6ccccc6cc54)c3)cc2)c2ccc(-c3ccc4ccccc4c3)cc2)cc1]

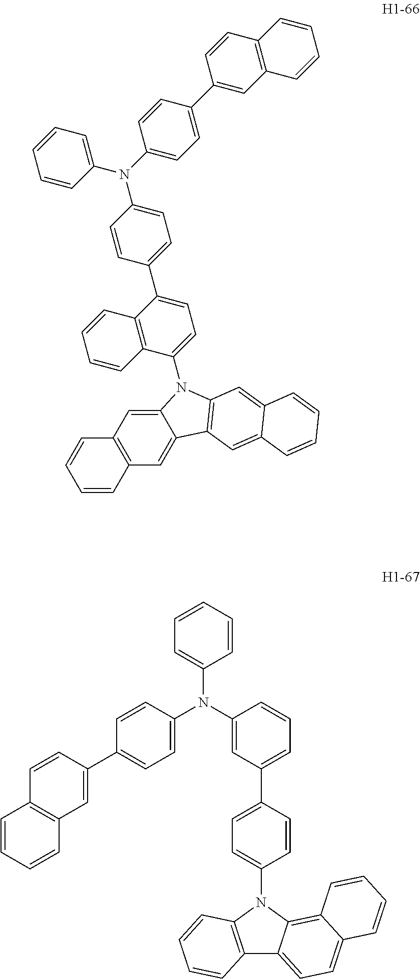 [SMILES: c1ccc(N(c2ccc(-c3ccc4ccccc4c3)cc2)c2ccc(-c3ccc(-n4c5cc6ccccc6cc5c5cc6ccccc6cc54)c4ccccc34)cc2)cc1 ; c1ccc(N(c2ccc(-c3ccc4ccccc4c3)cc2)c2cccc(-c3ccc(-n4c5ccccc5c5ccc6ccccc6c54)cc3)c2)cc1]

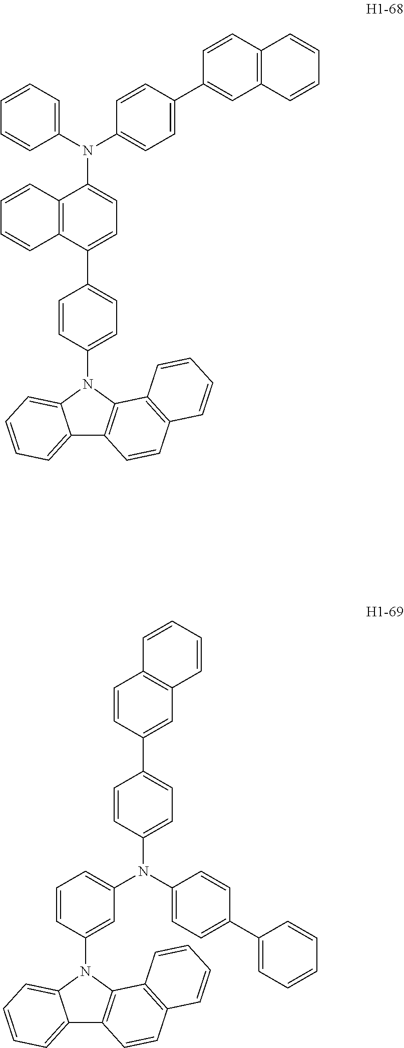 [SMILES: c1ccc(N(c2ccc(-c3ccc4ccccc4c3)cc2)c2ccc(-c3ccc(-n4c5ccccc5c5ccc6ccccc6c54)cc3)c3ccccc23)cc1 ; c1ccc(-c2ccc(N(c3ccc(-c4ccc5ccccc5c4)cc3)c3cccc(-n4c5ccccc5c5ccc6ccccc6c54)c3)cc2)cc1]

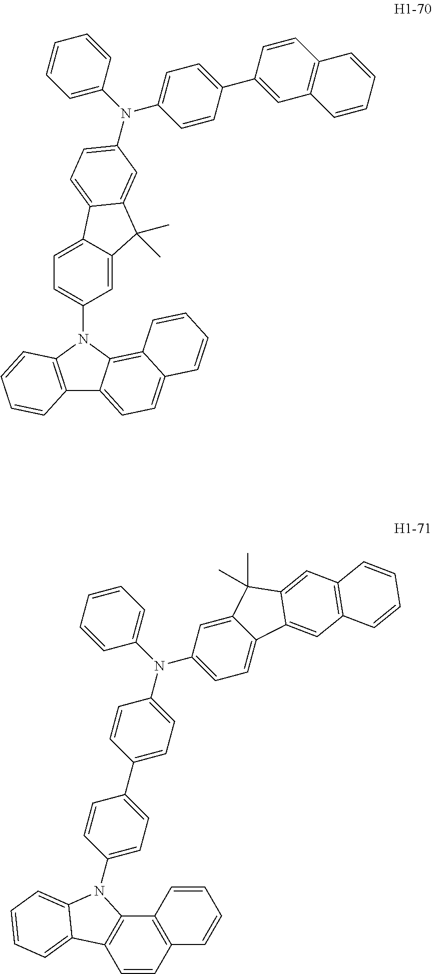 [SMILES: CC1(C)c2cc(N(c3ccccc3)c3ccc(-c4ccc5ccccc5c4)cc3)ccc2-c2ccc(-n3c4ccccc4c4ccc5ccccc5c43)cc21 ; CC1(C)c2cc(N(c3ccccc3)c3ccc(-c4ccc(-n5c6ccccc6c6ccc7ccccc7c65)cc4)cc3)ccc2-c2cc3ccccc3cc21]

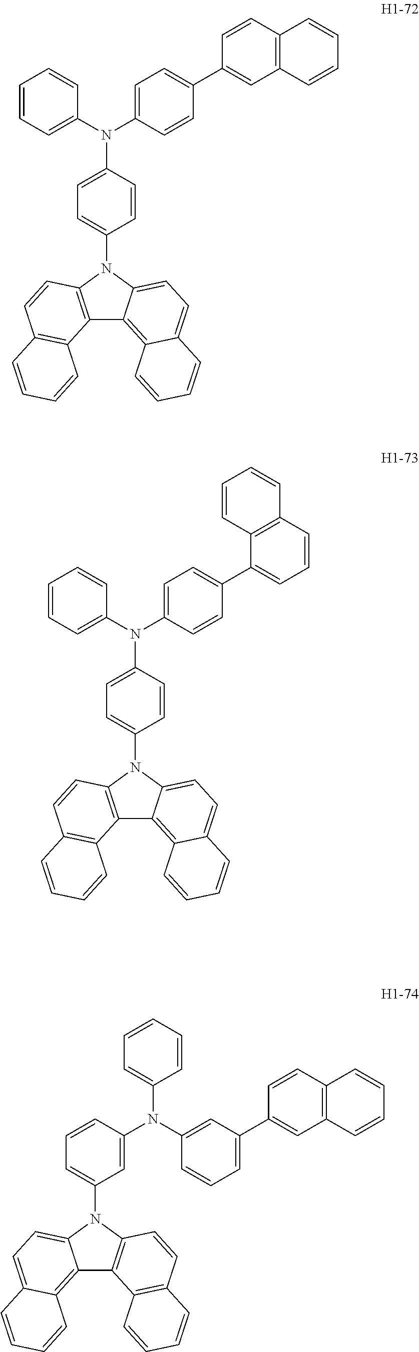 [SMILES: c1ccc(N(c2ccc(-c3ccc4ccccc4c3)cc2)c2ccc(-n3c4ccc5ccccc5c4c4c5ccccc5ccc43)cc2)cc1 ; c1ccc(N(c2ccc(-c3cccc4ccccc34)cc2)c2ccc(-n3c4ccc5ccccc5c4c4c5ccccc5ccc43)cc2)cc1 ; c1ccc(N(c2cccc(-c3ccc4ccccc4c3)c2)c2cccc(-n3c4ccc5ccccc5c4c4c5ccccc5ccc43)c2)cc1]

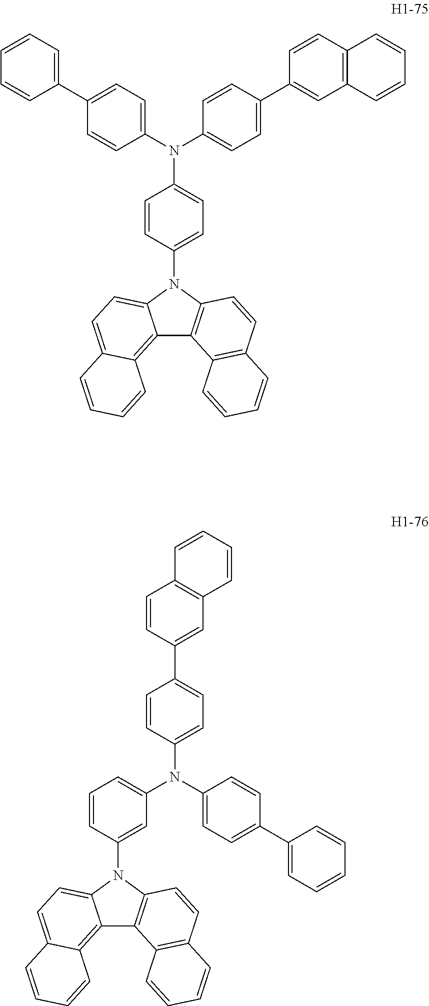 [SMILES: c1ccc(-c2ccc(N(c3ccc(-c4ccc5ccccc5c4)cc3)c3ccc(-n4c5ccc6ccccc6c5c5c6ccccc6ccc54)cc3)cc2)cc1 ; c1ccc(-c2ccc(N(c3ccc(-c4ccc5ccccc5c4)cc3)c3cccc(-n4c5ccc6ccccc6c5c5c6ccccc6ccc54)c3)cc2)cc1]

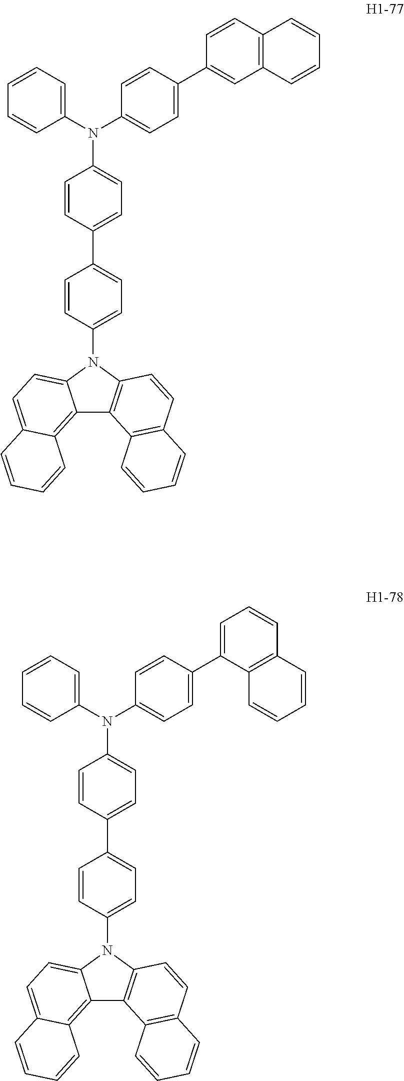 [SMILES: c1ccc(N(c2ccc(-c3ccc(-n4c5ccc6ccccc6c5c5c6ccccc6ccc54)cc3)cc2)c2ccc(-c3ccc4ccccc4c3)cc2)cc1 ; c1ccc(N(c2ccc(-c3ccc(-n4c5ccc6ccccc6c5c5c6ccccc6ccc54)cc3)cc2)c2ccc(-c3cccc4ccccc34)cc2)cc1]

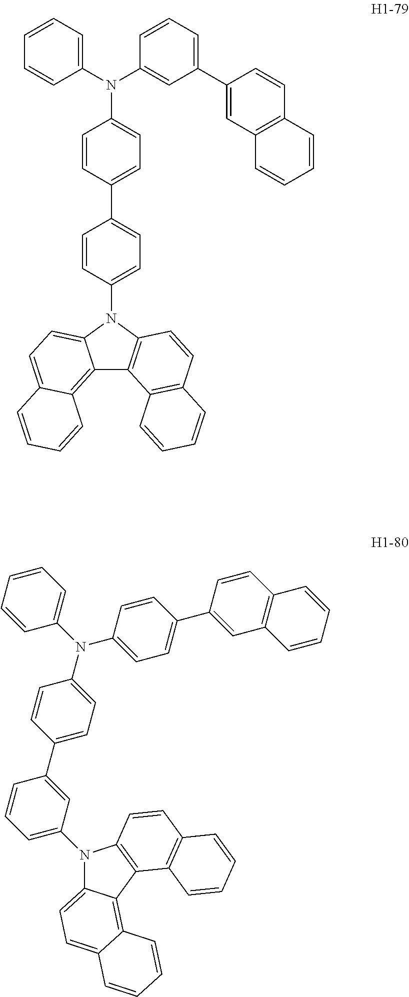 [SMILES: c1ccc(N(c2ccc(-c3ccc(-n4c5ccc6ccccc6c5c5c6ccccc6ccc54)cc3)cc2)c2cccc(-c3ccc4ccccc4c3)c2)cc1 ; c1ccc(N(c2ccc(-c3cccc(-n4c5ccc6ccccc6c5c5c6ccccc6ccc54)c3)cc2)c2ccc(-c3ccc4ccccc4c3)cc2)cc1]

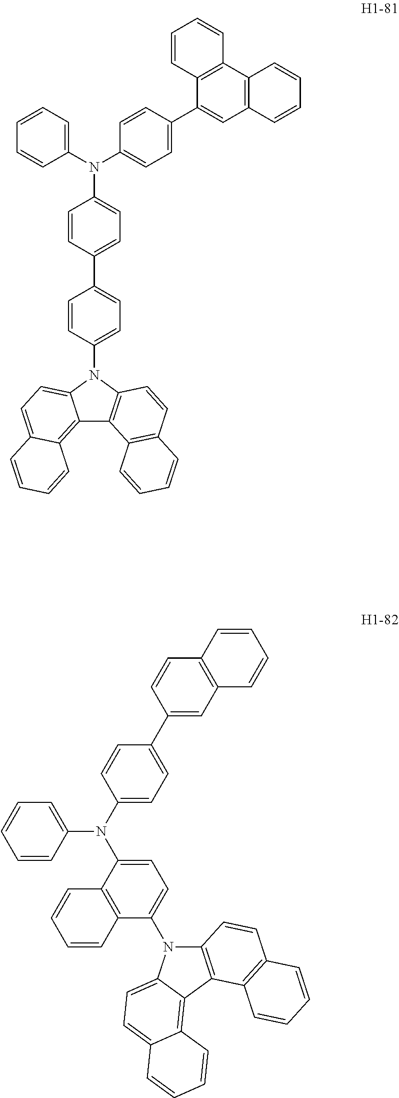 [SMILES: c1ccc(N(c2ccc(-c3ccc(-n4c5ccc6ccccc6c5c5c6ccccc6ccc54)cc3)cc2)c2ccc(-c3cc4ccccc4c4ccccc34)cc2)cc1 ; c1ccc(N(c2ccc(-c3ccc4ccccc4c3)cc2)c2ccc(-n3c4ccc5ccccc5c4c4c5ccccc5ccc43)c3ccccc23)cc1]

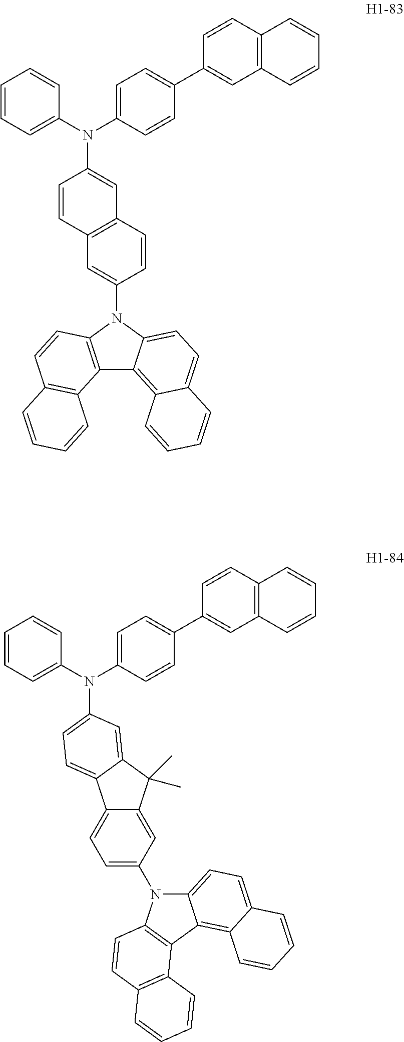 [SMILES: c1ccc(N(c2ccc(-c3ccc4ccccc4c3)cc2)c2ccc3cc(-n4c5ccc6ccccc6c5c5c6ccccc6ccc54)ccc3c2)cc1 ; CC1(C)c2cc(N(c3ccccc3)c3ccc(-c4ccc5ccccc5c4)cc3)ccc2-c2ccc(-n3c4ccc5ccccc5c4c4c5ccccc5ccc43)cc21]

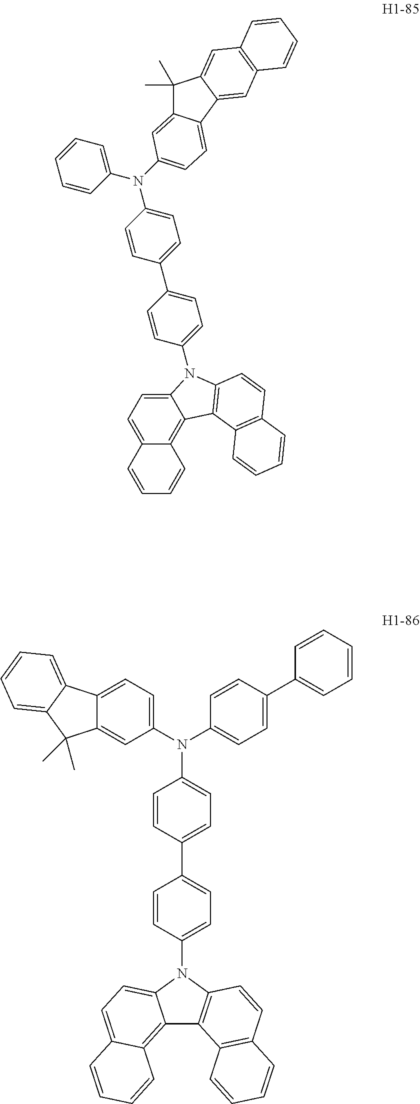 [SMILES: CC1(C)c2cc(N(c3ccccc3)c3ccc(-c4ccc(-n5c6ccc7ccccc7c6c6c7ccccc7ccc65)cc4)cc3)ccc2-c2cc3ccccc3cc21 ; CC1(C)c2ccccc2-c2ccc(N(c3ccc(-c4ccccc4)cc3)c3ccc(-c4ccc(-n5c6ccc7ccccc7c6c6c7ccccc7ccc65)cc4)cc3)cc21]

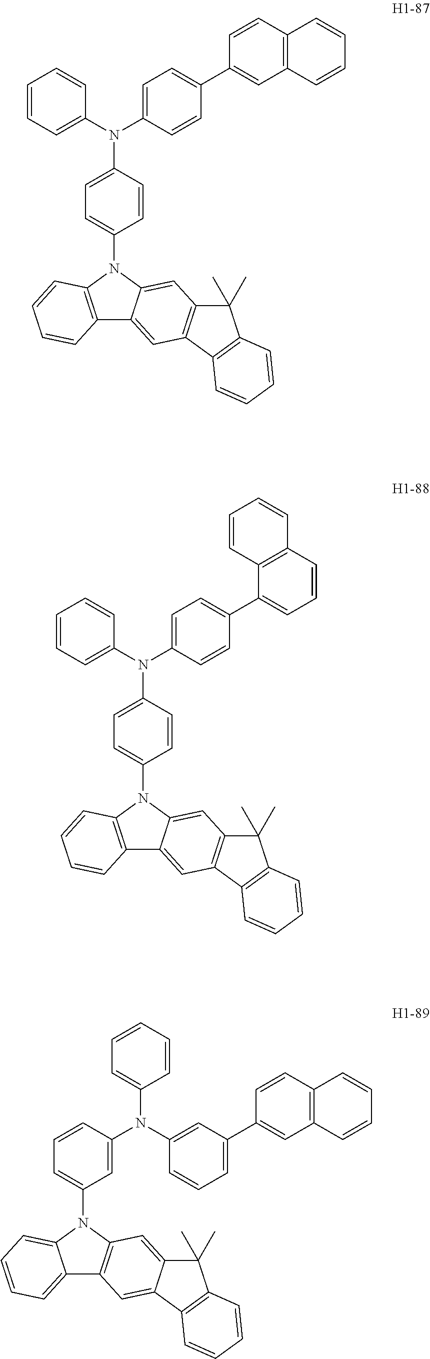 [SMILES: CC1(C)c2ccccc2-c2cc3c4ccccc4n(-c4ccc(N(c5ccccc5)c5ccc(-c6ccc7ccccc7c6)cc5)cc4)c3cc21 ; CC1(C)c2ccccc2-c2cc3c4ccccc4n(-c4ccc(N(c5ccccc5)c5ccc(-c6cccc7ccccc67)cc5)cc4)c3cc21 ; CC1(C)c2ccccc2-c2cc3c4ccccc4n(-c4cccc(N(c5ccccc5)c5cccc(-c6ccc7ccccc7c6)c5)c4)c3cc21]

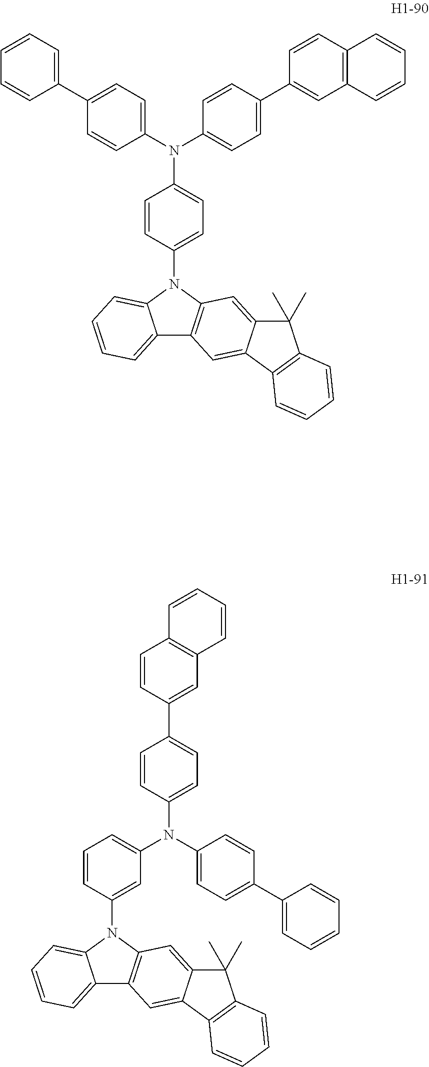 [SMILES: CC1(C)c2ccccc2-c2cc3c4ccccc4n(-c4ccc(N(c5ccc(-c6ccccc6)cc5)c5ccc(-c6ccc7ccccc7c6)cc5)cc4)c3cc21 ; CC1(C)c2ccccc2-c2cc3c4ccccc4n(-c4cccc(N(c5ccc(-c6ccccc6)cc5)c5ccc(-c6ccc7ccccc7c6)cc5)c4)c3cc21]

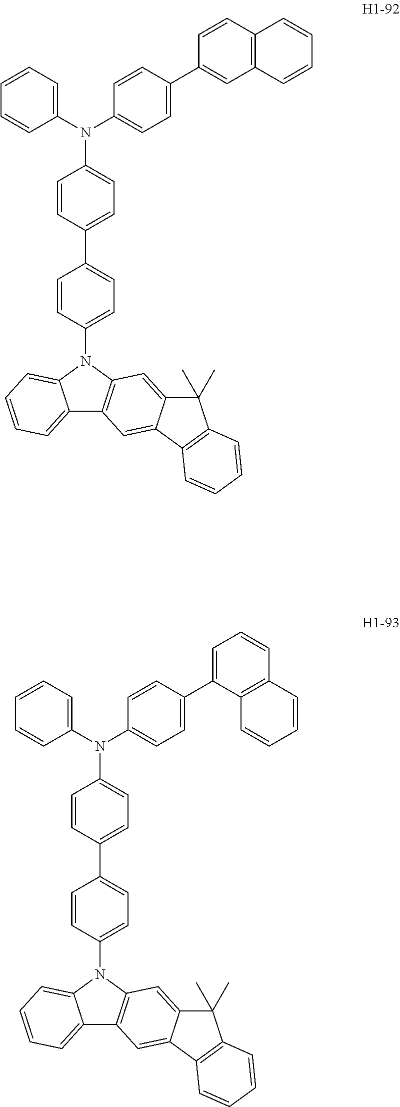 [SMILES: CC1(C)c2ccccc2-c2cc3c4ccccc4n(-c4ccc(-c5ccc(N(c6ccccc6)c6ccc(-c7ccc8ccccc8c7)cc6)cc5)cc4)c3cc21 ; CC1(C)c2ccccc2-c2cc3c4ccccc4n(-c4ccc(-c5ccc(N(c6ccccc6)c6ccc(-c7cccc8ccccc78)cc6)cc5)cc4)c3cc21]

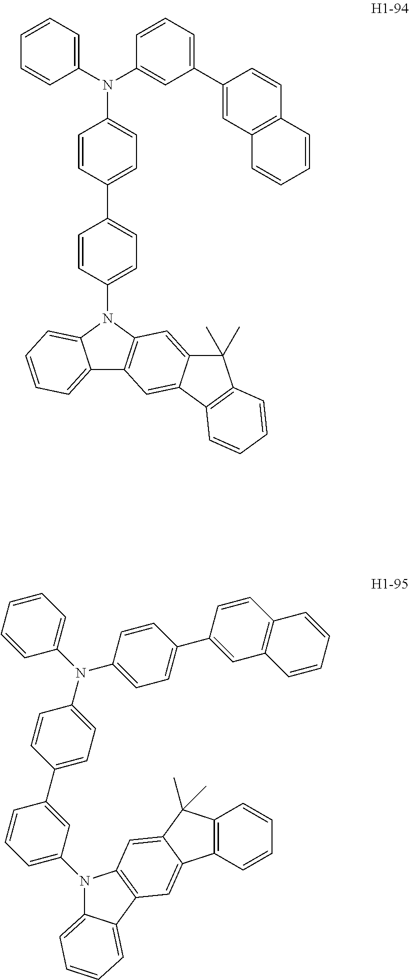 [SMILES: CC1(C)c2ccccc2-c2cc3c4ccccc4n(-c4ccc(-c5ccc(N(c6ccccc6)c6cccc(-c7ccc8ccccc8c7)c6)cc5)cc4)c3cc21 ; CC1(C)c2ccccc2-c2cc3c4ccccc4n(-c4cccc(-c5ccc(N(c6ccccc6)c6ccc(-c7ccc8ccccc8c7)cc6)cc5)c4)c3cc21]

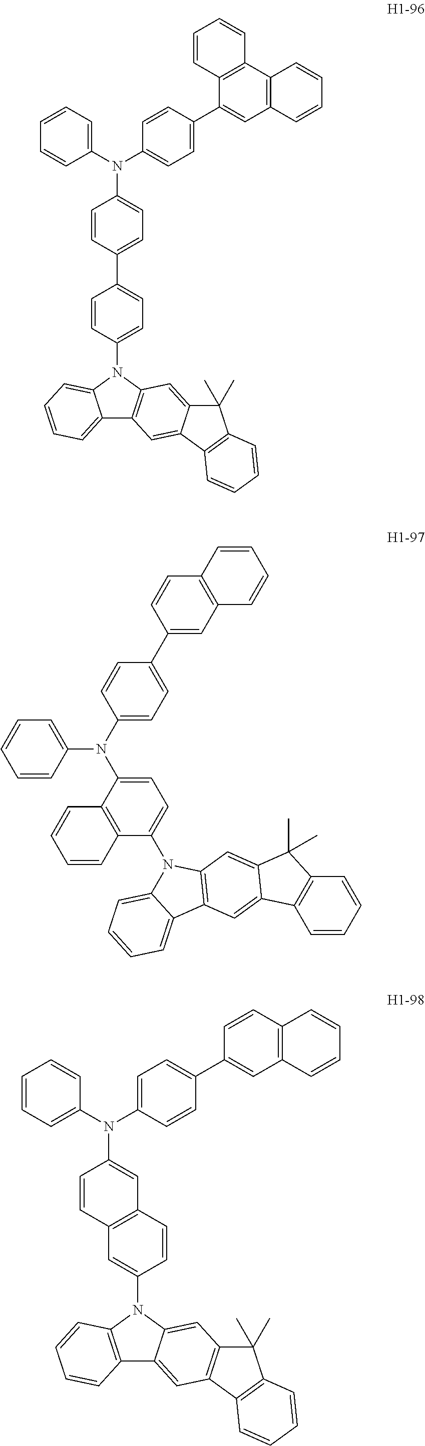 [SMILES: CC1(C)c2ccccc2-c2cc3c4ccccc4n(-c4ccc(-c5ccc(N(c6ccccc6)c6ccc(-c7cc8ccccc8c8ccccc78)cc6)cc5)cc4)c3cc21 ; CC1(C)c2ccccc2-c2cc3c4ccccc4n(-c4ccc(N(c5ccccc5)c5ccc(-c6ccc7ccccc7c6)cc5)c5ccccc45)c3cc21 ; CC1(C)c2ccccc2-c2cc3c4ccccc4n(-c4ccc5cc(N(c6ccccc6)c6ccc(-c7ccc8ccccc8c7)cc6)ccc5c4)c3cc21]

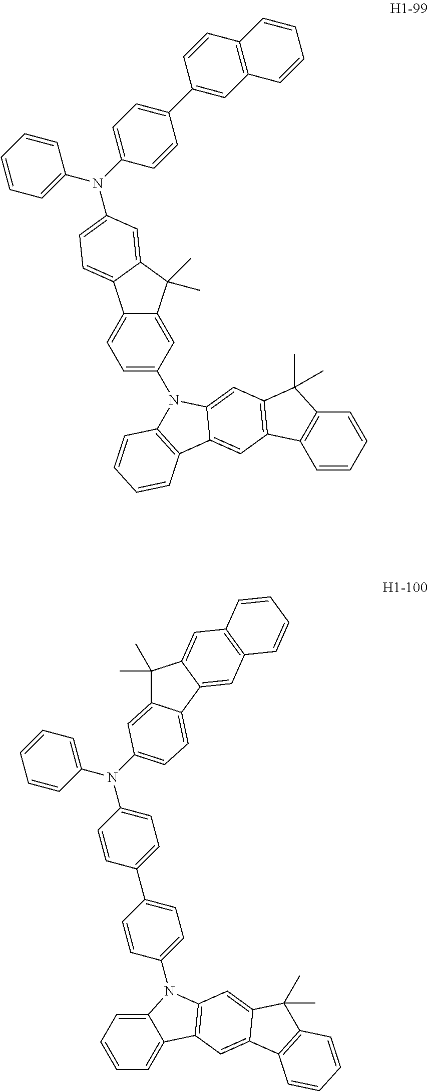 [SMILES: CC1(C)c2cc(N(c3ccccc3)c3ccc(-c4ccc5ccccc5c4)cc3)ccc2-c2ccc(-n3c4ccccc4c4cc5c(cc43)C(C)(C)c3ccccc3-5)cc21 ; CC1(C)c2cc(N(c3ccccc3)c3ccc(-c4ccc(-n5c6ccccc6c6cc7c(cc65)C(C)(C)c5ccccc5-7)cc4)cc3)ccc2-c2cc3ccccc3cc21]

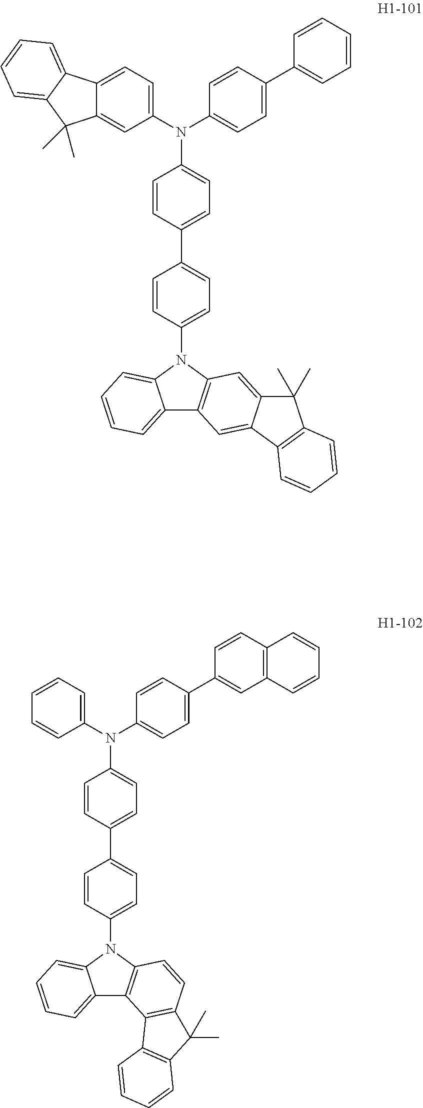 [SMILES: CC1(C)c2ccccc2-c2ccc(N(c3ccc(-c4ccccc4)cc3)c3ccc(-c4ccc(-n5c6ccccc6c6cc7c(cc65)C(C)(C)c5ccccc5-7)cc4)cc3)cc21 ; CC1(C)c2ccccc2-c2c1ccc1c2c2ccccc2n1-c1ccc(-c2ccc(N(c3ccccc3)c3ccc(-c4ccc5ccccc5c4)cc3)cc2)cc1]

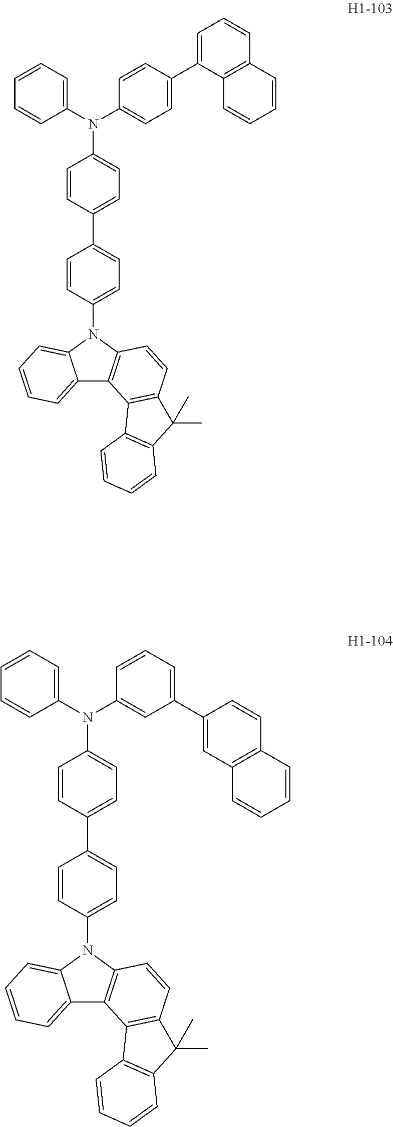 [SMILES: CC1(C)c2ccccc2-c2c1ccc1c2c2ccccc2n1-c1ccc(-c2ccc(N(c3ccccc3)c3ccc(-c4cccc5ccccc45)cc3)cc2)cc1 ; CC1(C)c2ccccc2-c2c1ccc1c2c2ccccc2n1-c1ccc(-c2ccc(N(c3ccccc3)c3cccc(-c4ccc5ccccc5c4)c3)cc2)cc1]

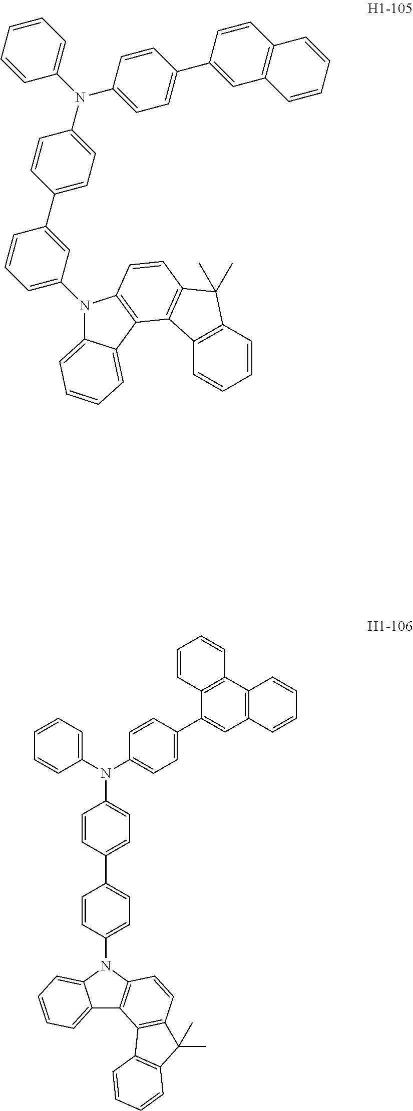 [SMILES: CC1(C)c2ccccc2-c2c1ccc1c2c2ccccc2n1-c1cccc(-c2ccc(N(c3ccccc3)c3ccc(-c4ccc5ccccc5c4)cc3)cc2)c1 ; CC1(C)c2ccccc2-c2c1ccc1c2c2ccccc2n1-c1ccc(-c2ccc(N(c3ccccc3)c3ccc(-c4cc5ccccc5c5ccccc45)cc3)cc2)cc1]

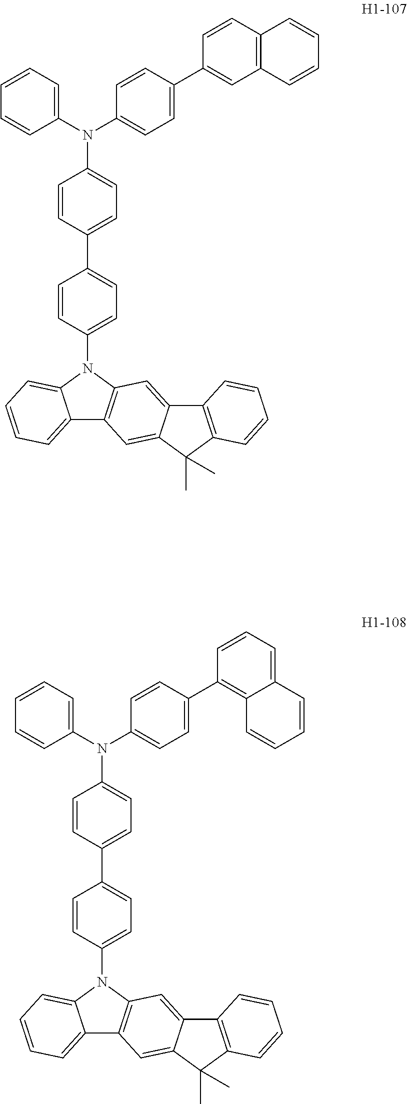 [SMILES: CC1(C)c2ccccc2-c2cc3c(cc21)c1ccccc1n3-c1ccc(-c2ccc(N(c3ccccc3)c3ccc(-c4ccc5ccccc5c4)cc3)cc2)cc1 ; CC1(C)c2ccccc2-c2cc3c(cc21)c1ccccc1n3-c1ccc(-c2ccc(N(c3ccccc3)c3ccc(-c4cccc5ccccc45)cc3)cc2)cc1]

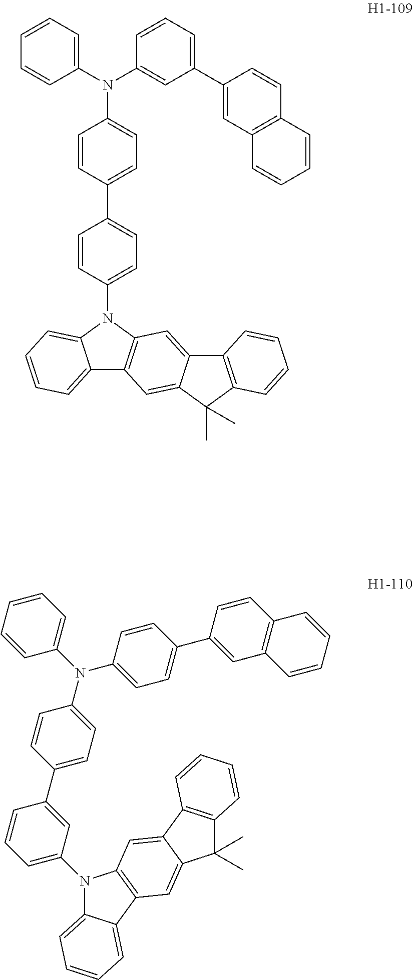 [SMILES: CC1(C)c2ccccc2-c2cc3c(cc21)c1ccccc1n3-c1ccc(-c2ccc(N(c3ccccc3)c3cccc(-c4ccc5ccccc5c4)c3)cc2)cc1 ; CC1(C)c2ccccc2-c2cc3c(cc21)c1ccccc1n3-c1cccc(-c2ccc(N(c3ccccc3)c3ccc(-c4ccc5ccccc5c4)cc3)cc2)c1]

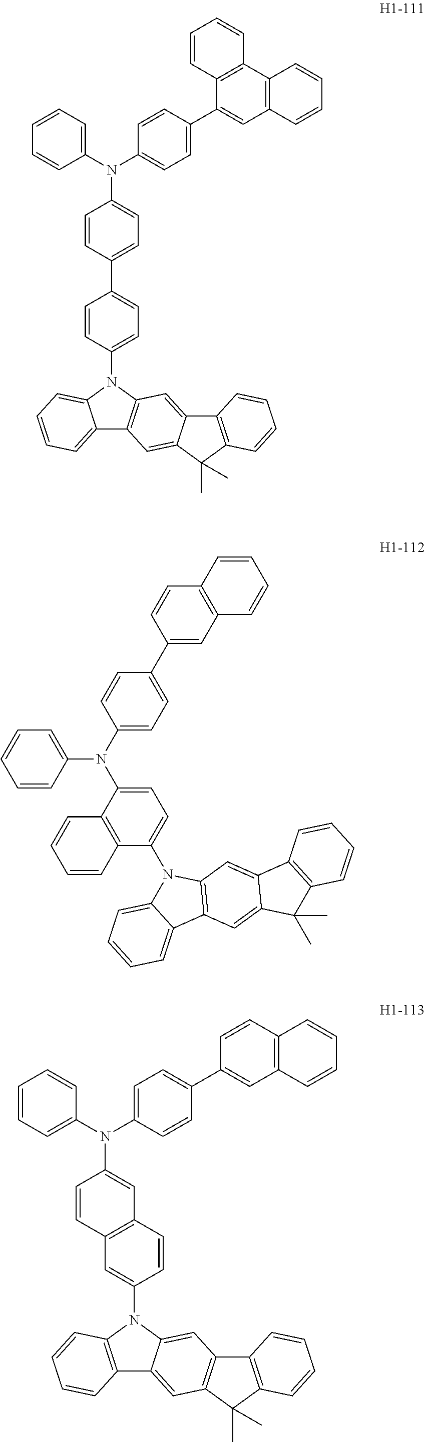 [SMILES: CC1(C)c2ccccc2-c2cc3c(cc21)c1ccccc1n3-c1ccc(-c2ccc(N(c3ccccc3)c3ccc(-c4cc5ccccc5c5ccccc45)cc3)cc2)cc1 ; CC1(C)c2ccccc2-c2cc3c(cc21)c1ccccc1n3-c1ccc(N(c2ccccc2)c2ccc(-c3ccc4ccccc4c3)cc2)c2ccccc12 ; CC1(C)c2ccccc2-c2cc3c(cc21)c1ccccc1n3-c1ccc2cc(N(c3ccccc3)c3ccc(-c4ccc5ccccc5c4)cc3)ccc2c1]

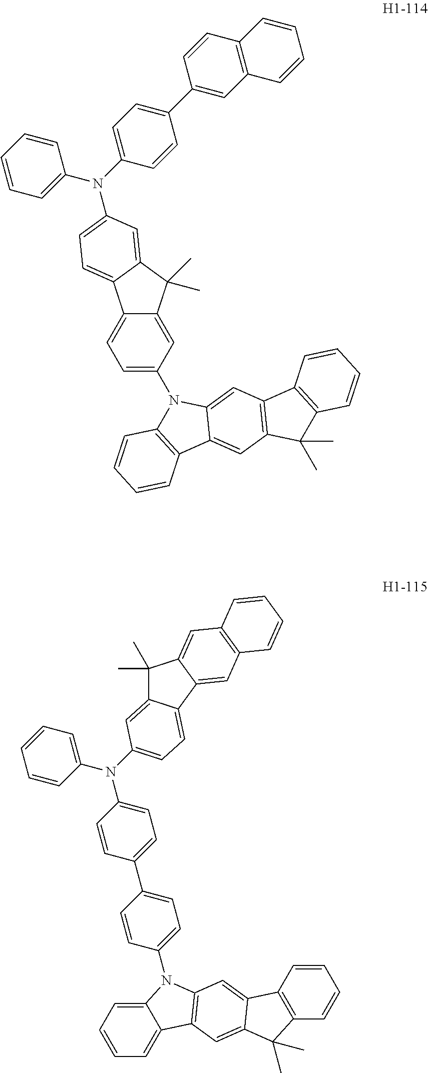 [SMILES: CC1(C)c2cc(N(c3ccccc3)c3ccc(-c4ccc5ccccc5c4)cc3)ccc2-c2ccc(-n3c4ccccc4c4cc5c(cc43)-c3ccccc3C5(C)C)cc21 ; CC1(C)c2cc(N(c3ccccc3)c3ccc(-c4ccc(-n5c6ccccc6c6cc7c(cc65)-c5ccccc5C7(C)C)cc4)cc3)ccc2-c2cc3ccccc3cc21]

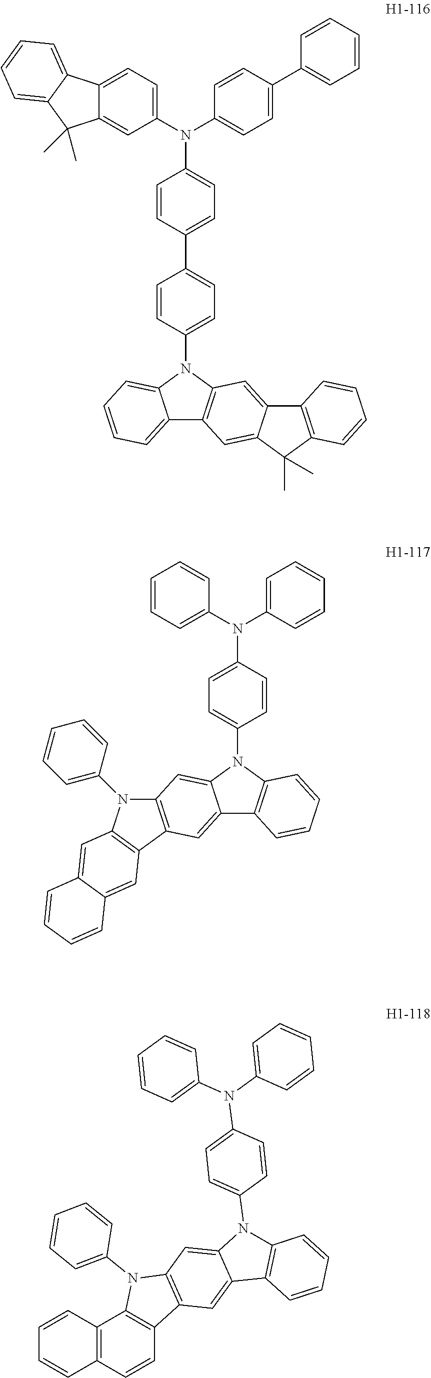 [SMILES: CC1(C)c2ccccc2-c2ccc(N(c3ccc(-c4ccccc4)cc3)c3ccc(-c4ccc(-n5c6ccccc6c6cc7c(cc65)-c5ccccc5C7(C)C)cc4)cc3)cc21 ; c1ccc(N(c2ccccc2)c2ccc(-n3c4ccccc4c4cc5c6cc7ccccc7cc6n(-c6ccccc6)c5cc43)cc2)cc1 ; c1ccc(N(c2ccccc2)c2ccc(-n3c4ccccc4c4cc5c6ccc7ccccc7c6n(-c6ccccc6)c5cc43)cc2)cc1]

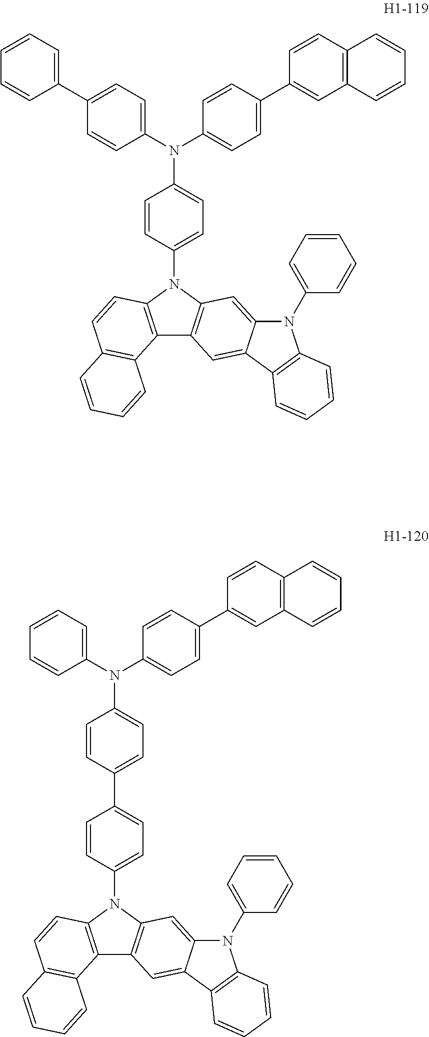 [SMILES: c1ccc(-c2ccc(N(c3ccc(-c4ccc5ccccc5c4)cc3)c3ccc(-n4c5cc6c(cc5c5c7ccccc7ccc54)c4ccccc4n6-c4ccccc4)cc3)cc2)cc1 ; c1ccc(N(c2ccc(-c3ccc(-n4c5cc6c(cc5c5c7ccccc7ccc54)c4ccccc4n6-c4ccccc4)cc3)cc2)c2ccc(-c3ccc4ccccc4c3)cc2)cc1]

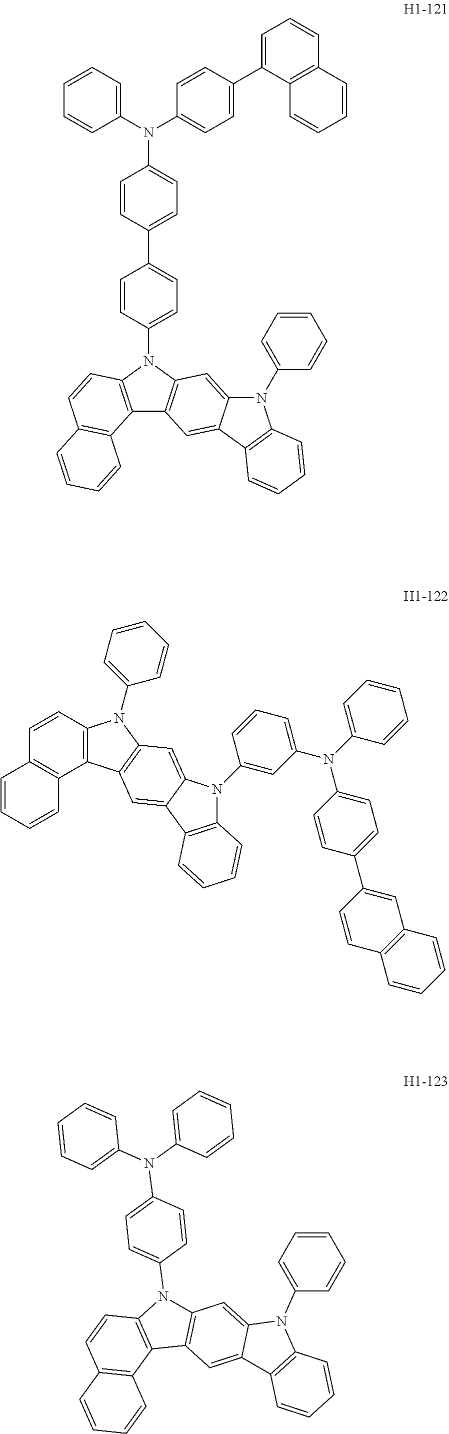 [SMILES: c1ccc(N(c2ccc(-c3ccc(-n4c5cc6c(cc5c5c7ccccc7ccc54)c4ccccc4n6-c4ccccc4)cc3)cc2)c2ccc(-c3cccc4ccccc34)cc2)cc1 ; c1ccc(N(c2ccc(-c3ccc4ccccc4c3)cc2)c2cccc(-n3c4ccccc4c4cc5c6c7ccccc7ccc6n(-c6ccccc6)c5cc43)c2)cc1 ; c1ccc(N(c2ccccc2)c2ccc(-n3c4cc5c(cc4c4c6ccccc6ccc43)c3ccccc3n5-c3ccccc3)cc2)cc1]

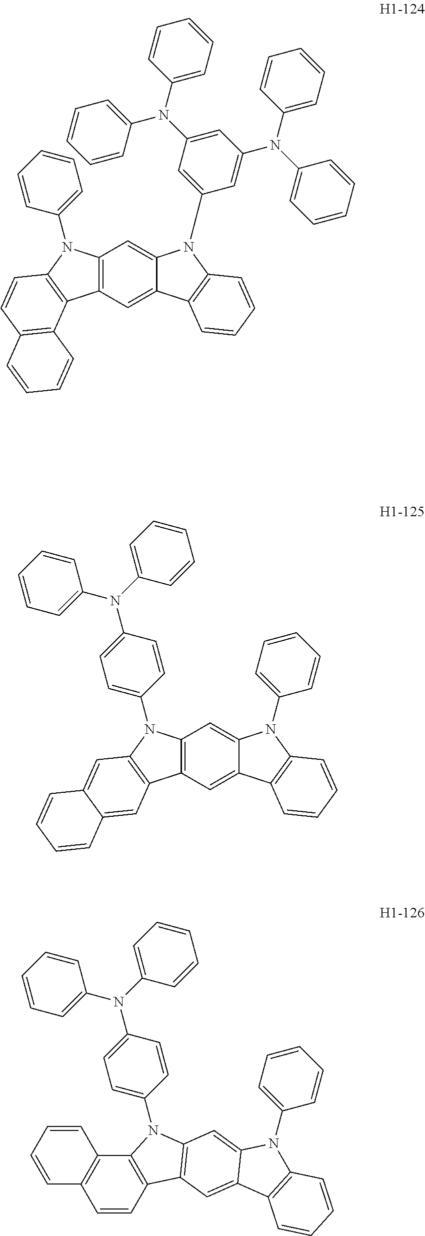 [SMILES: c1ccc(N(c2ccccc2)c2cc(N(c3ccccc3)c3ccccc3)cc(-n3c4ccccc4c4cc5c6c7ccccc7ccc6n(-c6ccccc6)c5cc43)c2)cc1 ; c1ccc(N(c2ccccc2)c2ccc(-n3c4cc5ccccc5cc4c4cc5c6ccccc6n(-c6ccccc6)c5cc43)cc2)cc1 ; c1ccc(N(c2ccccc2)c2ccc(-n3c4cc5c(cc4c4ccc6ccccc6c43)c3ccccc3n5-c3ccccc3)cc2)cc1]

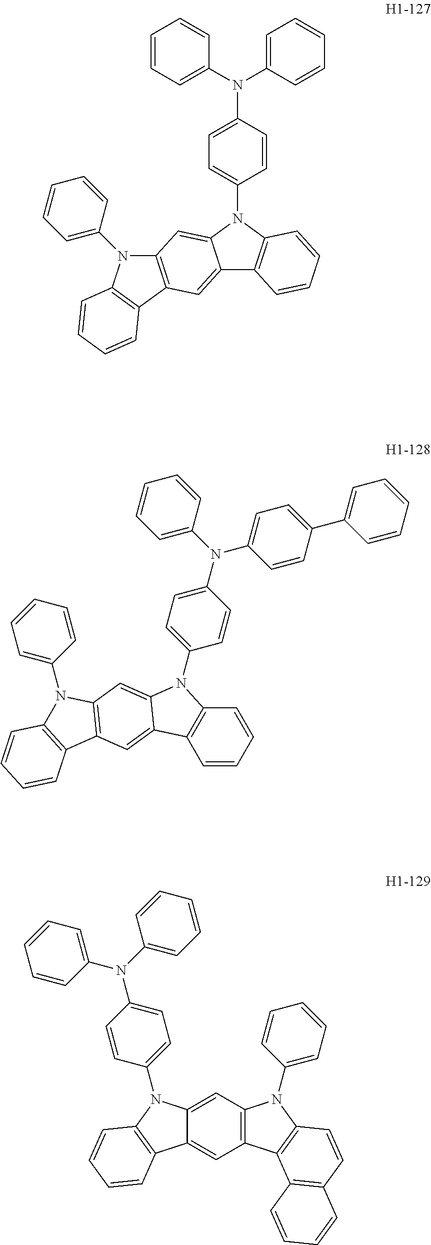 [SMILES: c1ccc(N(c2ccccc2)c2ccc(-n3c4ccccc4c4cc5c6ccccc6n(-c6ccccc6)c5cc43)cc2)cc1 ; c1ccc(-c2ccc(N(c3ccccc3)c3ccc(-n4c5ccccc5c5cc6c7ccccc7n(-c7ccccc7)c6cc54)cc3)cc2)cc1 ; c1ccc(N(c2ccccc2)c2ccc(-n3c4ccccc4c4cc5c6c7ccccc7ccc6n(-c6ccccc6)c5cc43)cc2)cc1]

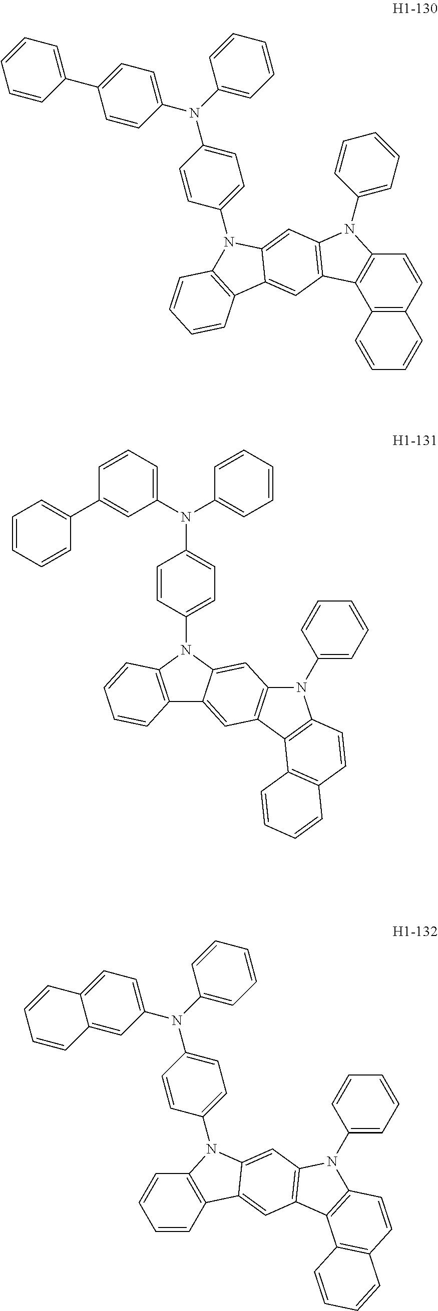 [SMILES: c1ccc(-c2ccc(N(c3ccccc3)c3ccc(-n4c5ccccc5c5cc6c7c8ccccc8ccc7n(-c7ccccc7)c6cc54)cc3)cc2)cc1 ; c1ccc(-c2cccc(N(c3ccccc3)c3ccc(-n4c5ccccc5c5cc6c7c8ccccc8ccc7n(-c7ccccc7)c6cc54)cc3)c2)cc1 ; c1ccc(N(c2ccc(-n3c4ccccc4c4cc5c6c7ccccc7ccc6n(-c6ccccc6)c5cc43)cc2)c2ccc3ccccc3c2)cc1]

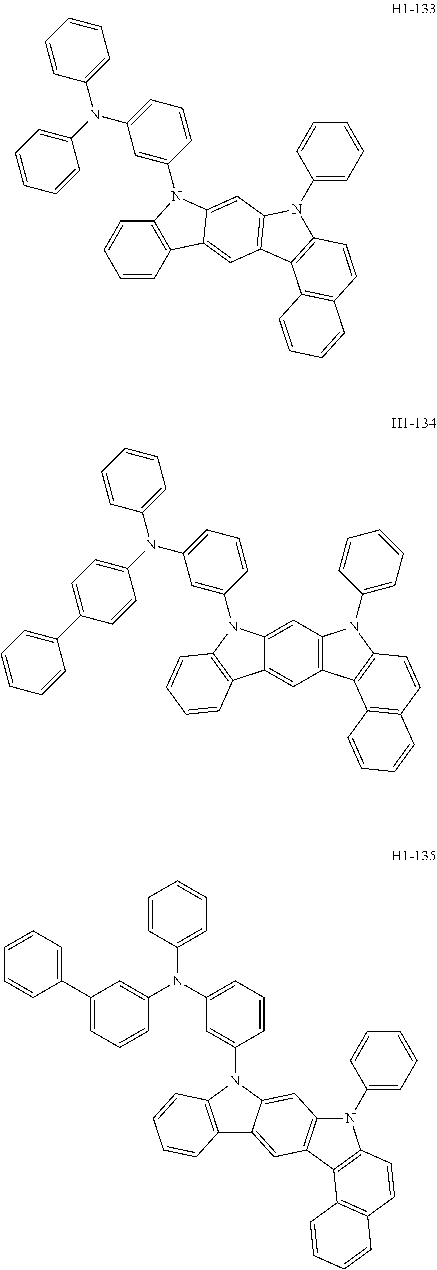 [SMILES: c1ccc(N(c2ccccc2)c2cccc(-n3c4ccccc4c4cc5c6c7ccccc7ccc6n(-c6ccccc6)c5cc43)c2)cc1 ; c1ccc(-c2ccc(N(c3ccccc3)c3cccc(-n4c5ccccc5c5cc6c7c8ccccc8ccc7n(-c7ccccc7)c6cc54)c3)cc2)cc1 ; c1ccc(-c2cccc(N(c3ccccc3)c3cccc(-n4c5ccccc5c5cc6c7c8ccccc8ccc7n(-c7ccccc7)c6cc54)c3)c2)cc1]

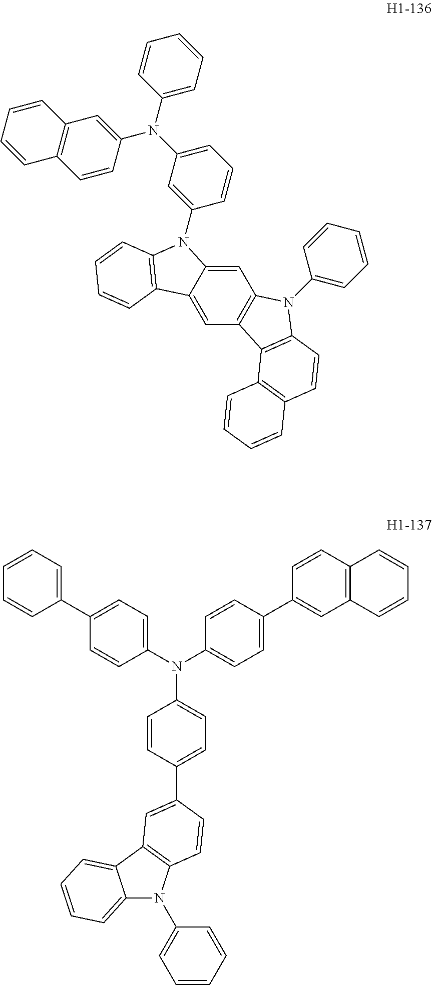 [SMILES: c1ccc(N(c2cccc(-n3c4ccccc4c4cc5c6c7ccccc7ccc6n(-c6ccccc6)c5cc43)c2)c2ccc3ccccc3c2)cc1 ; c1ccc(-c2ccc(N(c3ccc(-c4ccc5ccccc5c4)cc3)c3ccc(-c4ccc5c(c4)c4ccccc4n5-c4ccccc4)cc3)cc2)cc1]

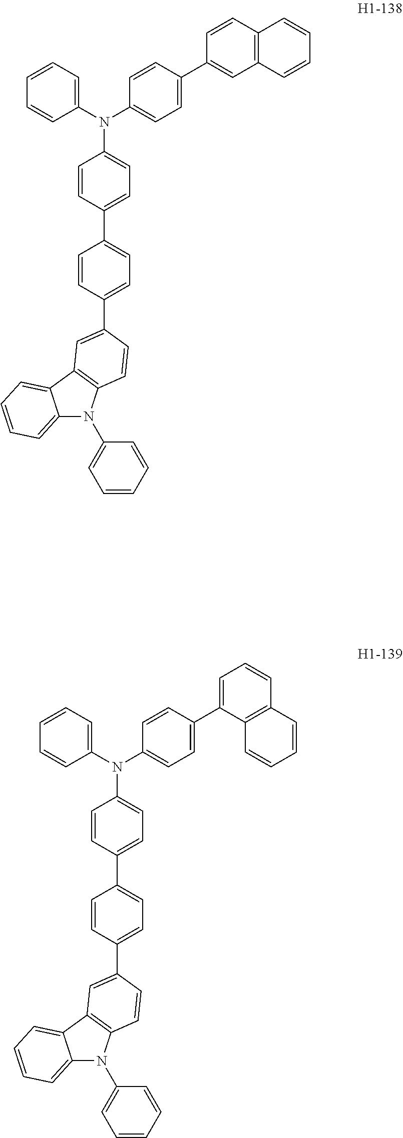 [SMILES: c1ccc(N(c2ccc(-c3ccc(-c4ccc5c(c4)c4ccccc4n5-c4ccccc4)cc3)cc2)c2ccc(-c3ccc4ccccc4c3)cc2)cc1 ; c1ccc(N(c2ccc(-c3ccc(-c4ccc5c(c4)c4ccccc4n5-c4ccccc4)cc3)cc2)c2ccc(-c3cccc4ccccc34)cc2)cc1]

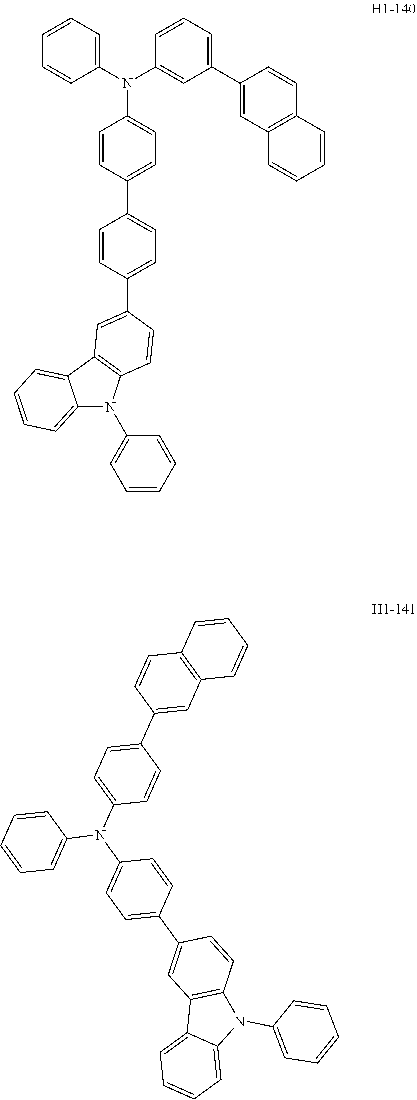 [SMILES: c1ccc(N(c2ccc(-c3ccc(-c4ccc5c(c4)c4ccccc4n5-c4ccccc4)cc3)cc2)c2cccc(-c3ccc4ccccc4c3)c2)cc1 ; c1ccc(N(c2ccc(-c3ccc4ccccc4c3)cc2)c2ccc(-c3ccc4c(c3)c3ccccc3n4-c3ccccc3)cc2)cc1]

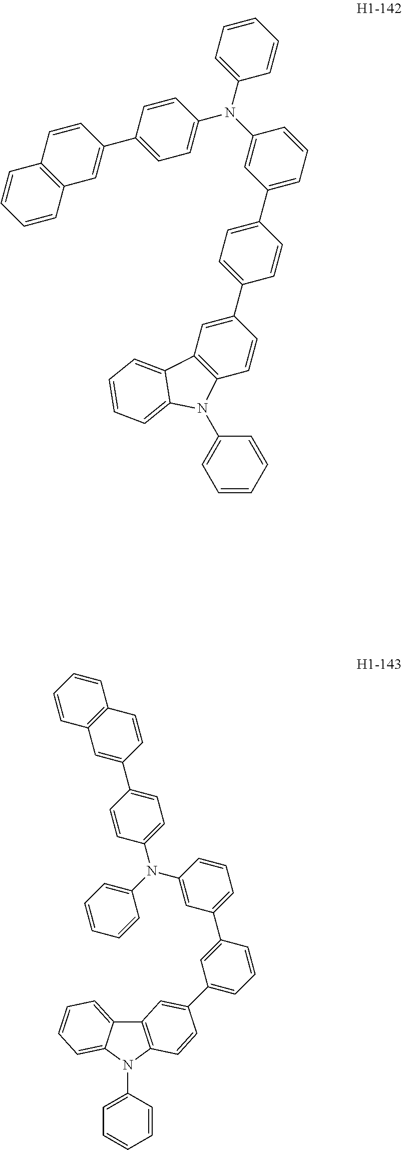 [SMILES: c1ccc(N(c2ccc(-c3ccc4ccccc4c3)cc2)c2cccc(-c3ccc(-c4ccc5c(c4)c4ccccc4n5-c4ccccc4)cc3)c2)cc1 ; c1ccc(N(c2ccc(-c3ccc4ccccc4c3)cc2)c2cccc(-c3cccc(-c4ccc5c(c4)c4ccccc4n5-c4ccccc4)c3)c2)cc1]

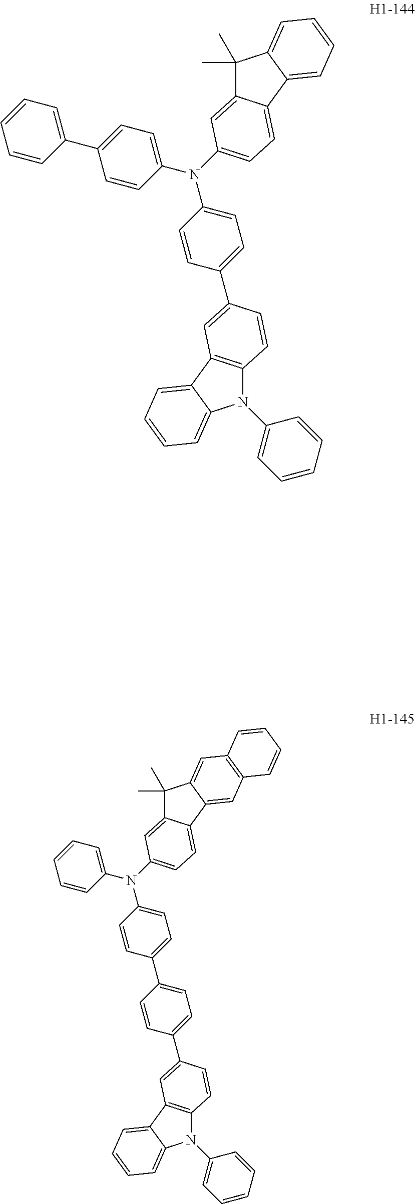 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2cc(N(c3ccccc3)c3ccc(-c4ccc(-c5ccc6c(c5)c5ccccc5n6-c5ccccc5)cc4)cc3)ccc2-c2cc3ccccc3cc21]

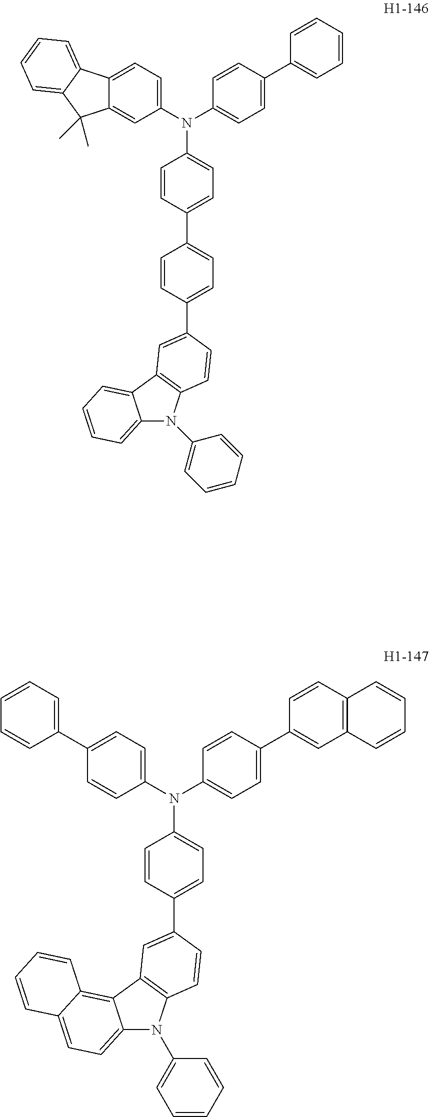 [SMILES: CC1(C)c2ccccc2-c2ccc(N(c3ccc(-c4ccccc4)cc3)c3ccc(-c4ccc(-c5ccc6c(c5)c5ccccc5n6-c5ccccc5)cc4)cc3)cc21 ; c1ccc(-c2ccc(N(c3ccc(-c4ccc5ccccc5c4)cc3)c3ccc(-c4ccc5c(c4)c4c6ccccc6ccc4n5-c4ccccc4)cc3)cc2)cc1]

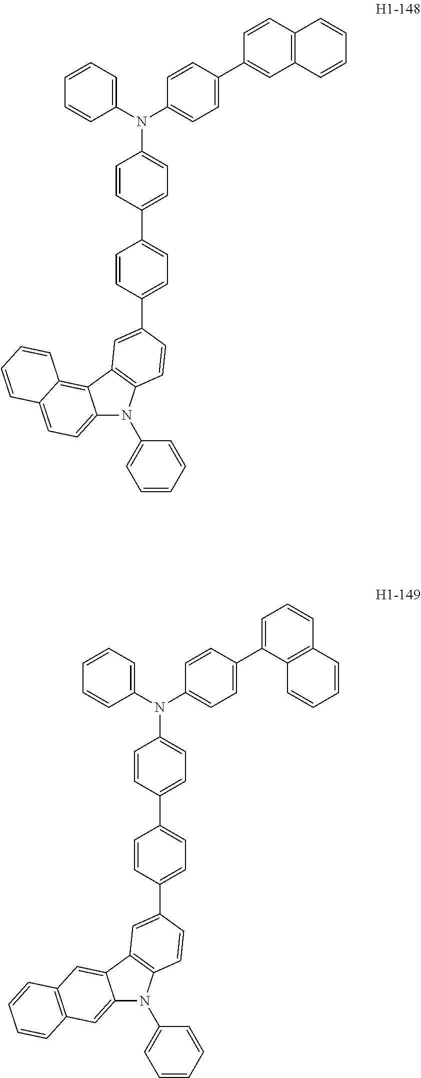 [SMILES: c1ccc(N(c2ccc(-c3ccc(-c4ccc5c(c4)c4c6ccccc6ccc4n5-c4ccccc4)cc3)cc2)c2ccc(-c3ccc4ccccc4c3)cc2)cc1 ; c1ccc(N(c2ccc(-c3ccc(-c4ccc5c(c4)c4cc6ccccc6cc4n5-c4ccccc4)cc3)cc2)c2ccc(-c3cccc4ccccc34)cc2)cc1]

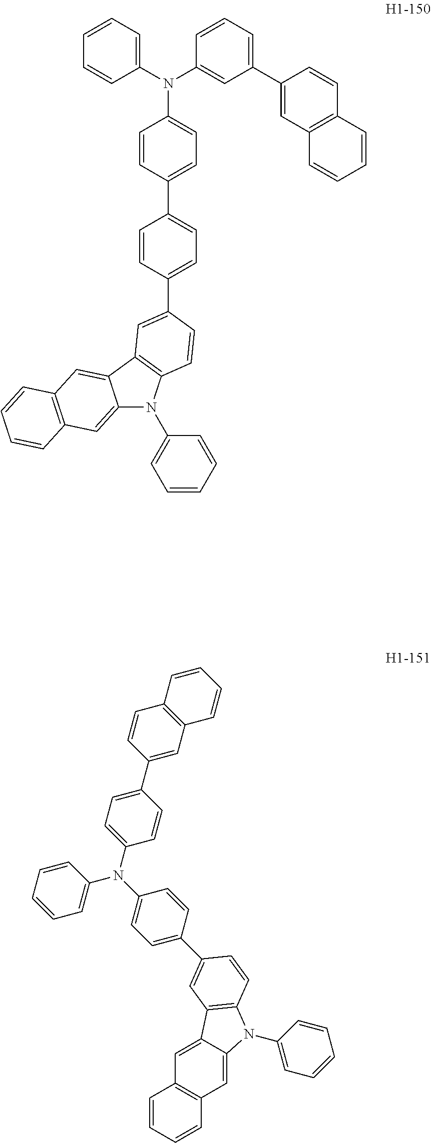 [SMILES: c1ccc(N(c2ccc(-c3ccc(-c4ccc5c(c4)c4cc6ccccc6cc4n5-c4ccccc4)cc3)cc2)c2cccc(-c3ccc4ccccc4c3)c2)cc1 ; c1ccc(N(c2ccc(-c3ccc4ccccc4c3)cc2)c2ccc(-c3ccc4c(c3)c3cc5ccccc5cc3n4-c3ccccc3)cc2)cc1]

As for the dopant comprised in the organic electroluminescent device according to the present disclosure, at least one phosphorescent or fluorescent dopant may be used, and at least one phosphorescent dopant is preferable. The phosphorescent dopant materials applied to the organic electroluminescent device according to the present disclosure are not particularly limited, but may be selected from metallated complex compounds of iridium (Ir), osmium (Os), copper (Cu), and platinum (Pt), may be preferably selected from ortho-metallated complex compounds of iridium (Ir), osmium (Os), copper (Cu), and platinum (Pt), and may be more preferably an ortho-metallated iridium complex compound.

The dopant comprised in the organic electroluminescent device of the present disclosure may be selected from the group consisting of the compounds represented by formulas 101 to 104 below, but is not limited thereto.

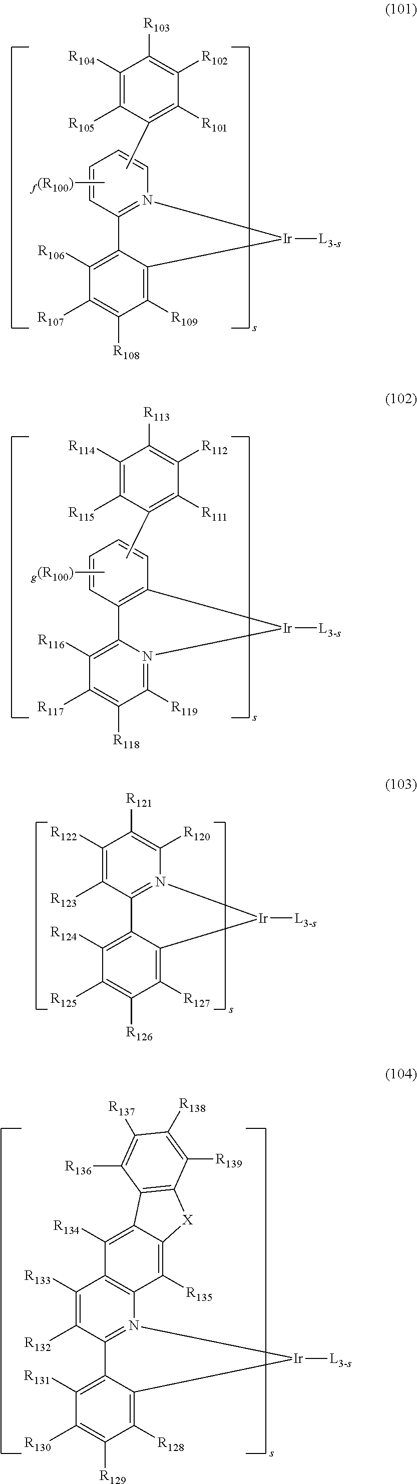

wherein L is selected from the following structures:

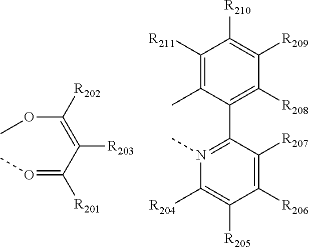

R₁₀₀, R₁₃₄, and R₁₃₅, each independently, represent hydrogen, deuterium, a substituted or unsubstituted (C1-C30)alkyl, or a substituted or unsubstituted (C3-C30)cycloalkyl;
R₁₀₁ to R₁₀₉ and R₁₁₁ to R₁₂₃, each independently, represent hydrogen, deuterium, a halogen, a (C1-C30)alkyl unsubstituted or substituted with deuterium or a halogen, a substituted or unsubstituted (C3-C30)cycloalkyl, a substituted or unsubstituted (C6-C30)aryl, a cyano, or a substituted or unsubstituted (C1-C30)alkoxy; R₁₀₆ to R₁₀₉ may be linked to an adjacent substituent to form a ring, e.g., a fluorene unsubstituted or substituted with an alkyl, a dibenzothiophene unsubstituted or substituted with an alkyl, or a dibenzofuran unsubstituted or substituted with an alkyl; R₁₂₀ to R₁₂₃ may be linked to an adjacent substituent to form a ring, e.g., a quinoline unsubstituted or substituted with at least one of an alkyl, an aryl, an aralkyl, and an alkylaryl;
R₁₂₄ to R₁₃₃ and R₁₃₆ to R₁₃₉, each independently, represent hydrogen, deuterium, a halogen, a substituted or unsubstituted (C1-C30)alkyl, or a substituted or unsubstituted (C6-C30)aryl; and R₁₂₄ to R₁₂₇ may be linked to an adjacent substituent to form a ring, e.g., a fluorene unsubstituted or substituted with an alkyl, a dibenzothiophene unsubstituted or substituted with an alkyl, or a dibenzofuran unsubstituted or substituted with an alkyl;
X represents CR₅₁R₅₂, O, or S;
R₅₁ and R₅₂, each independently, represent a substituted or unsubstituted (C1-C10)alkyl, or a substituted or unsubstituted (C6-C30)aryl;
R₂₀₁ to R₂₁₁, each independently, represent hydrogen, deuterium, a halogen, a (C1-C30)alkyl unsubstituted or substituted with deuterium or a halogen, a substituted or unsubstituted (C3-C30)cycloalkyl, or a (C6-C30)aryl unsubstituted or substituted with an alkyl or deuterium; and R₂₀₈ to R₂₁₁ may be linked to an adjacent substituent to form a ring, e.g., a fluorene unsubstituted or substituted with an alkyl, a dibenzothiophene unsubstituted or substituted with an alkyl, or a dibenzofuran unsubstituted or substituted with an alkyl;
f and g, each independently, represent an integer of 1 to 3; where f or g is an integer of 2 or more, each R₁₀₀ may be the same or different; and
s represents an integer of 1 to 3.

Specifically, the examples of the dopant compound are as follows, but not limited thereto.

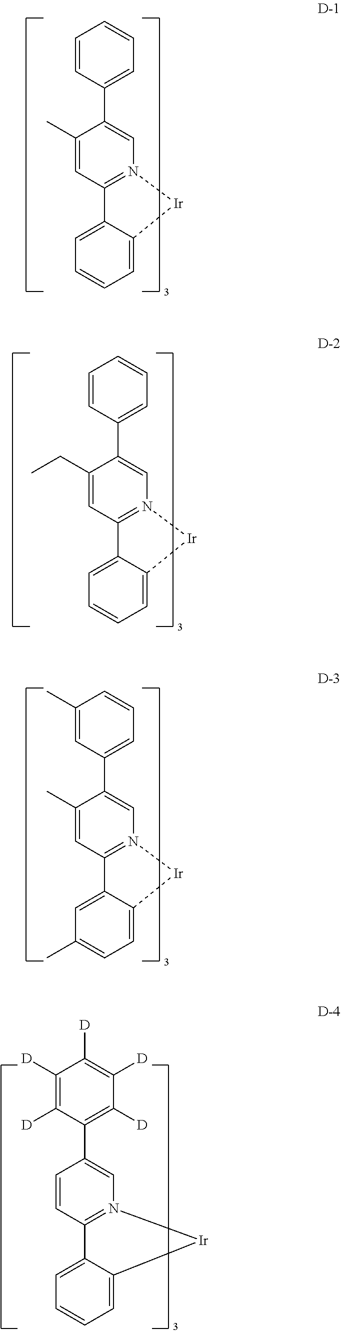

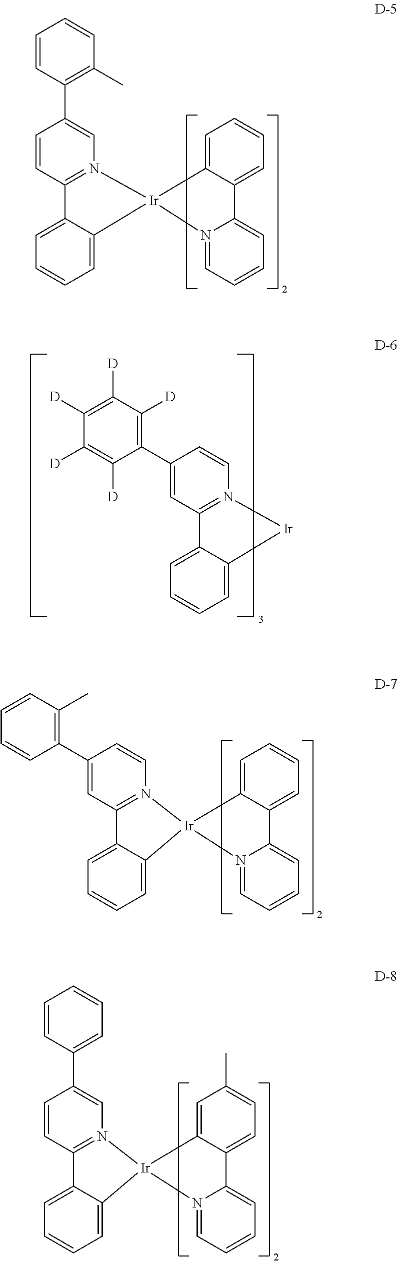

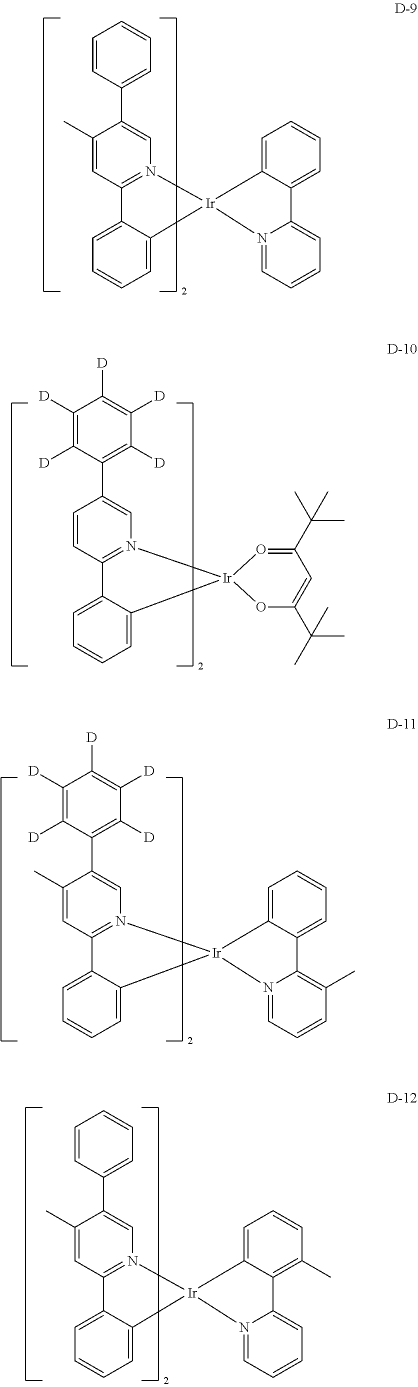

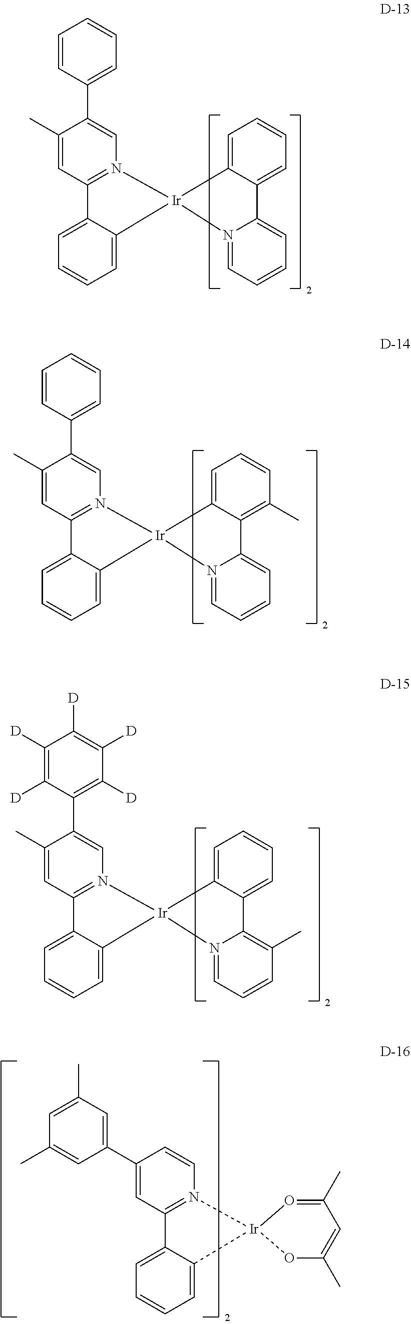

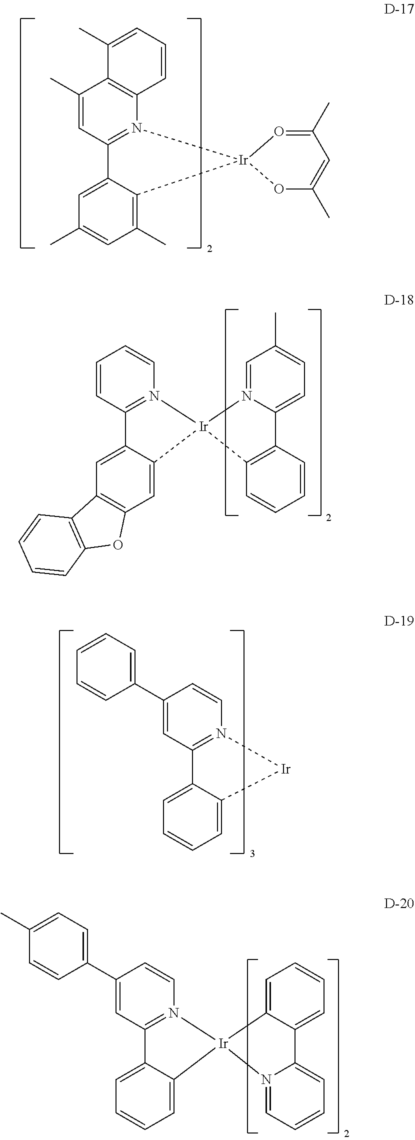

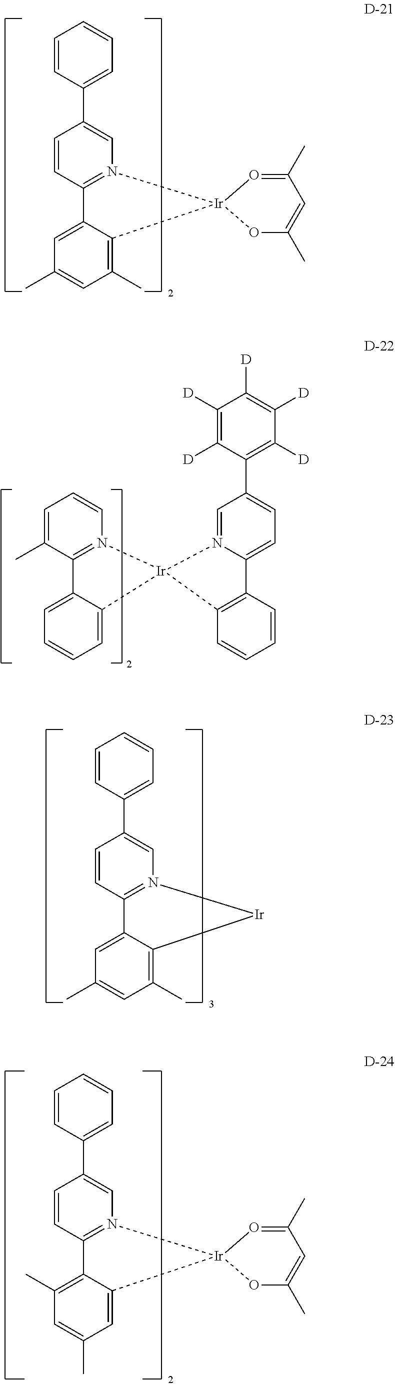

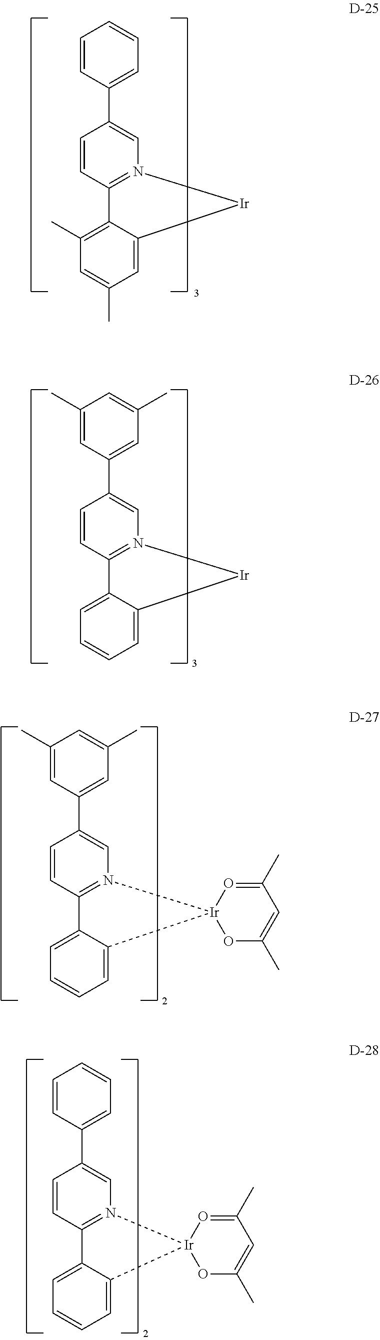

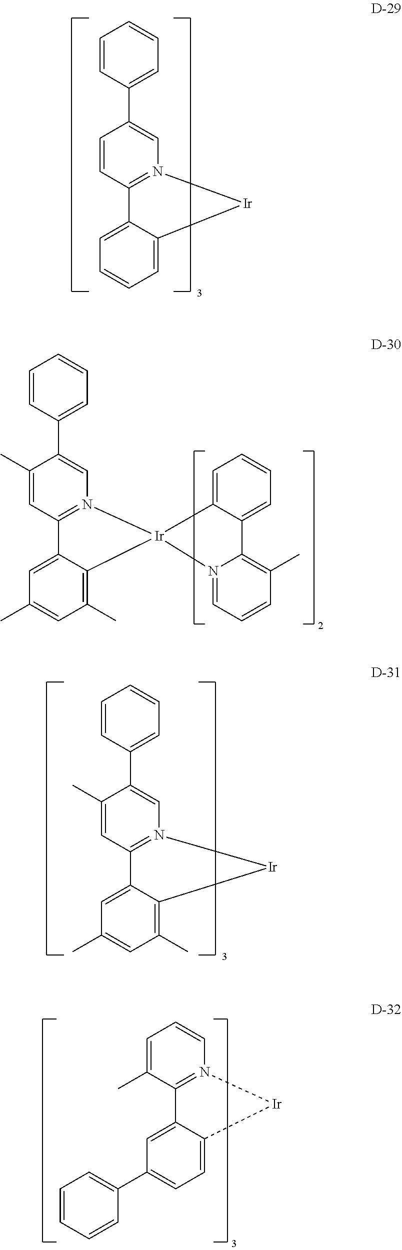

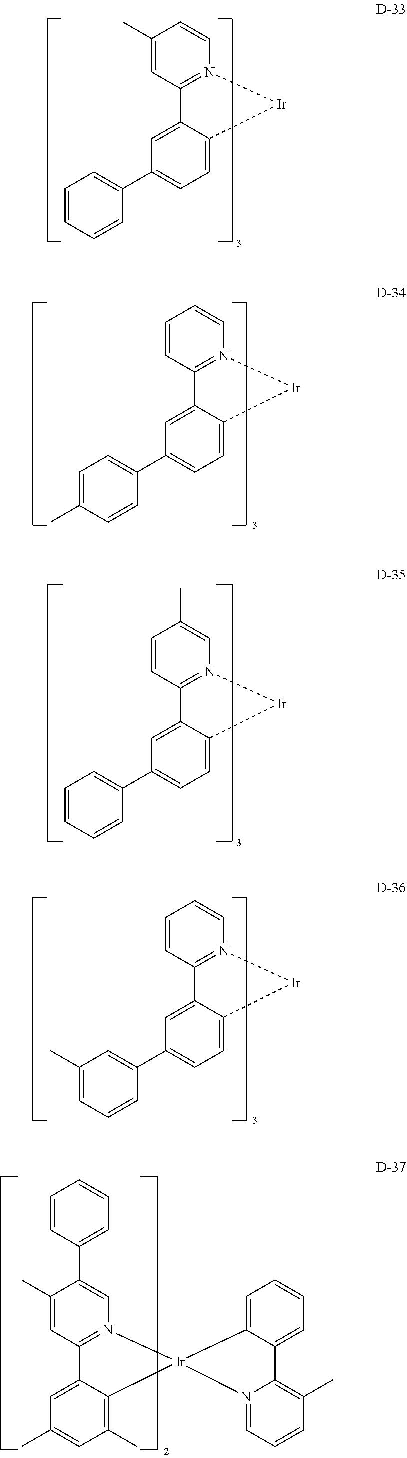

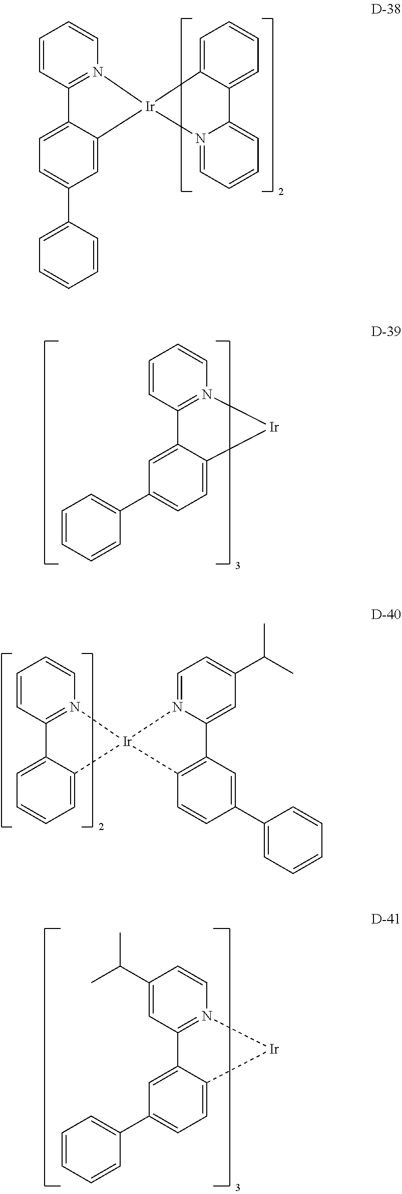

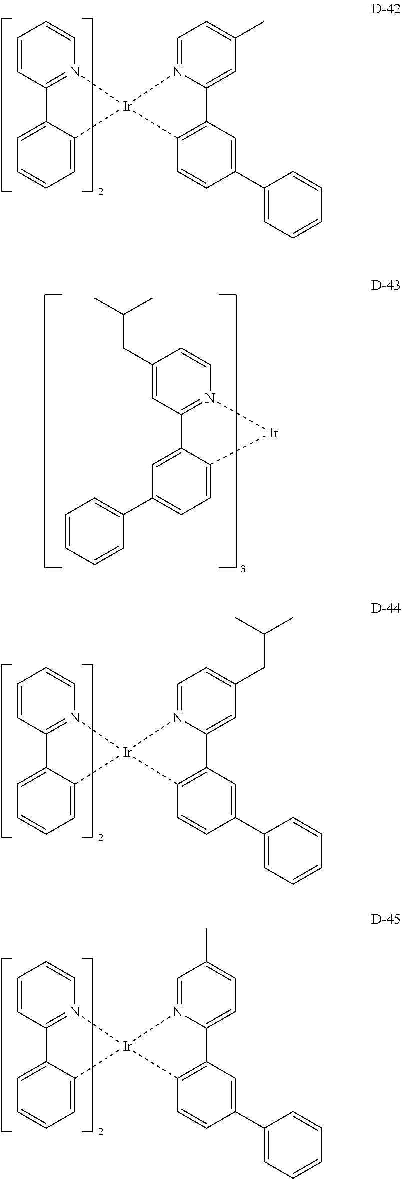

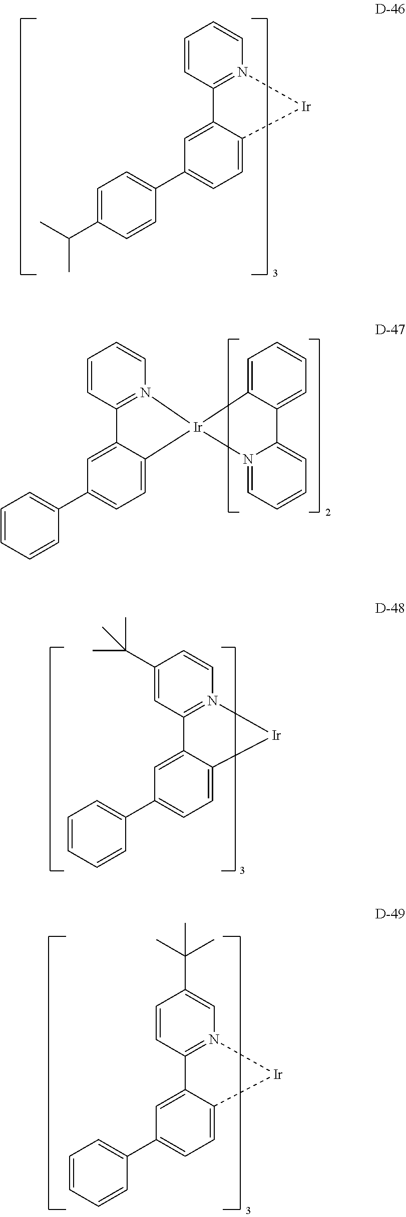

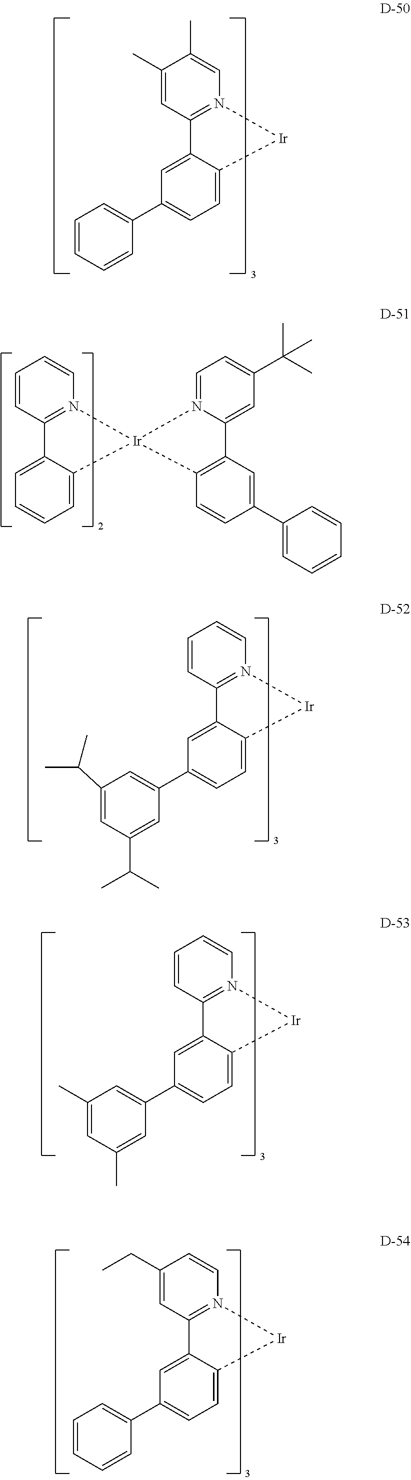

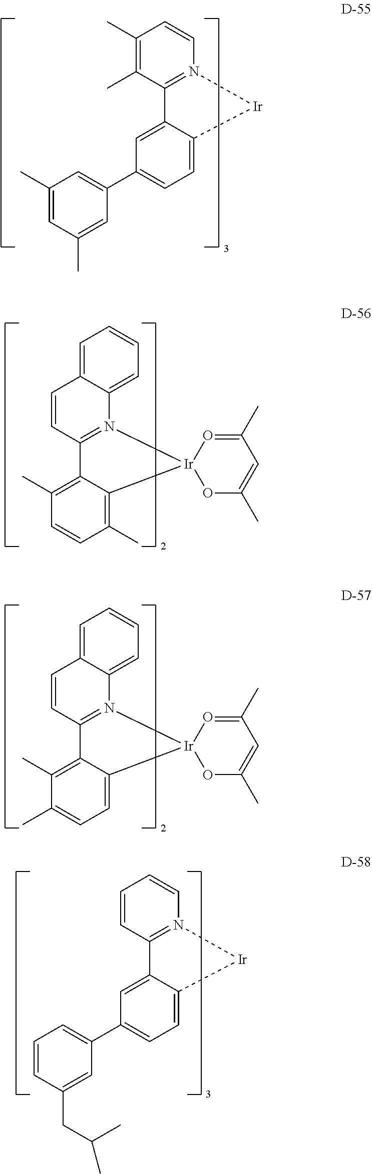

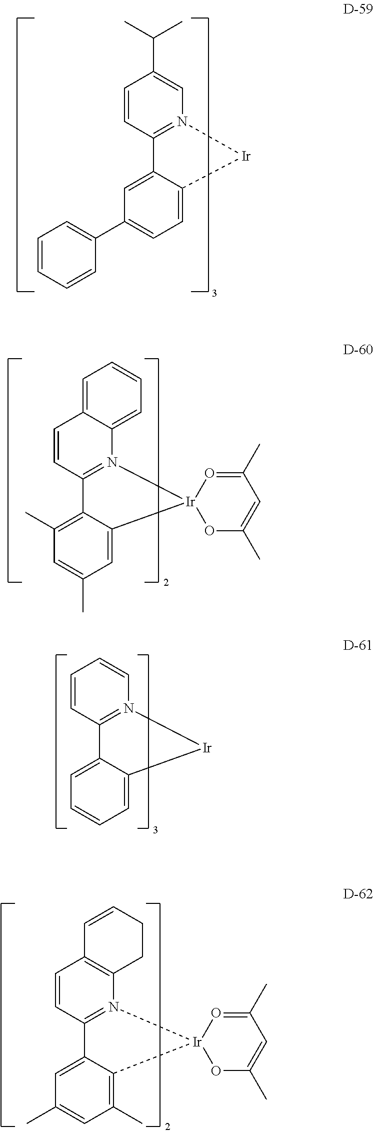

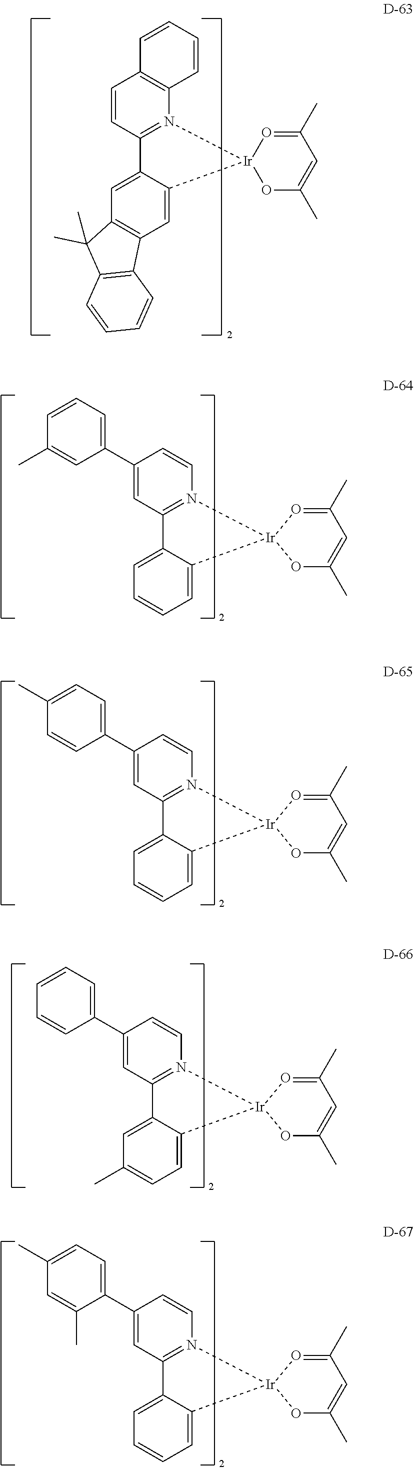

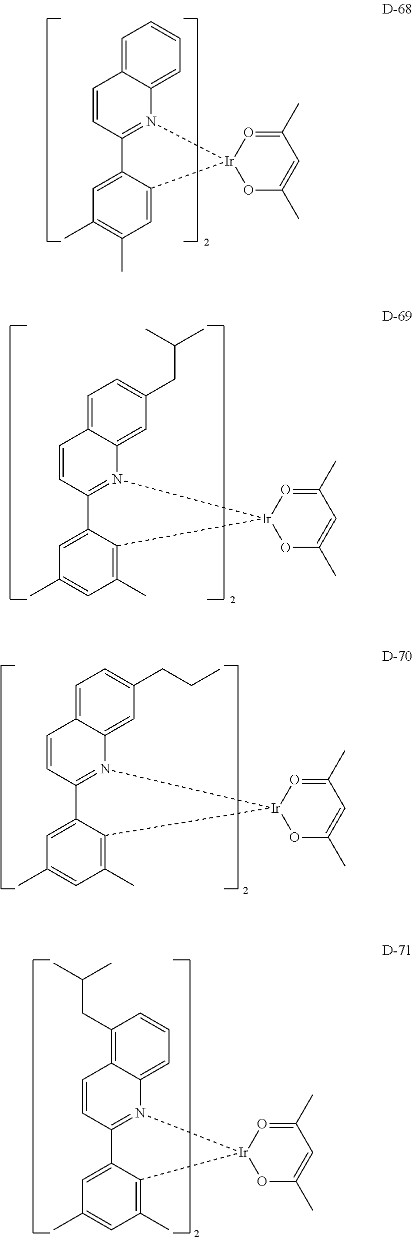

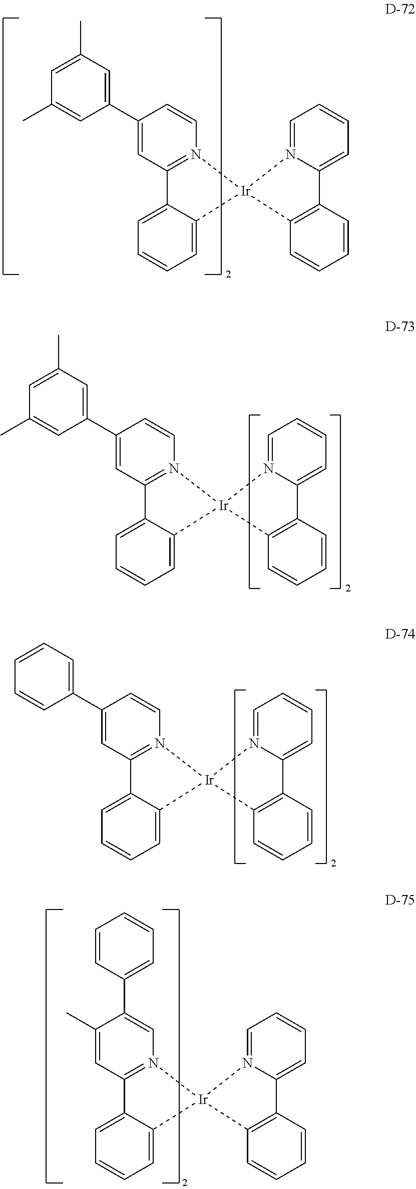

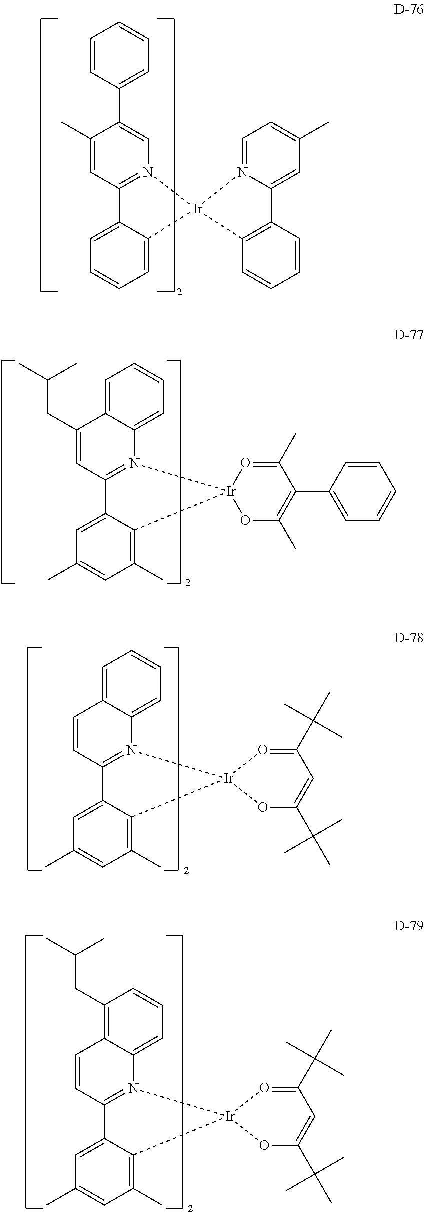

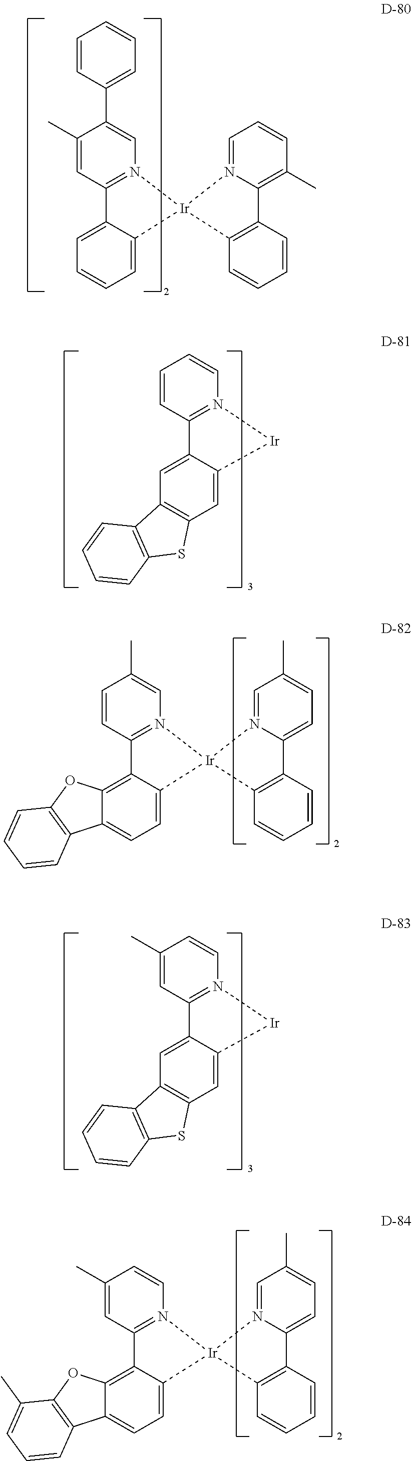

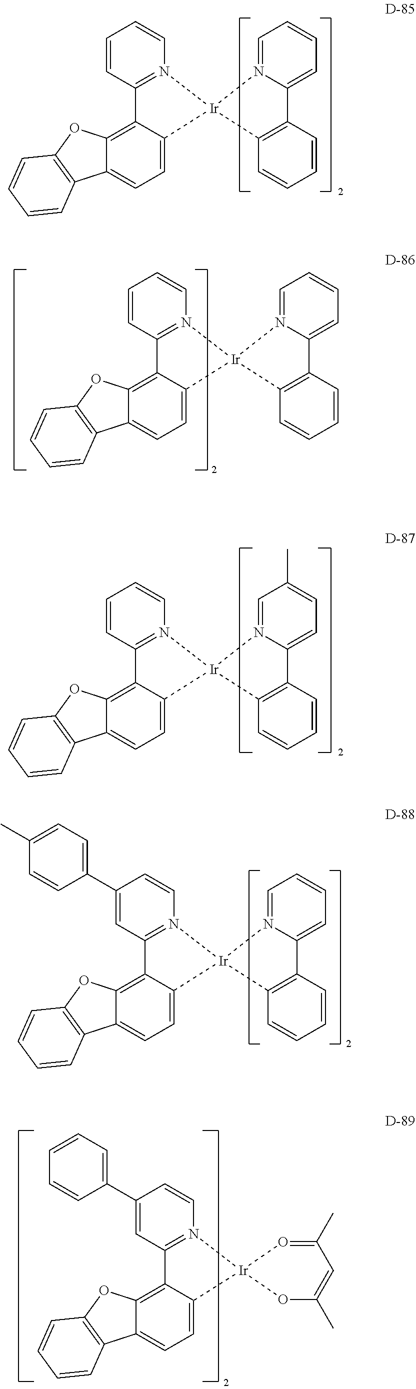

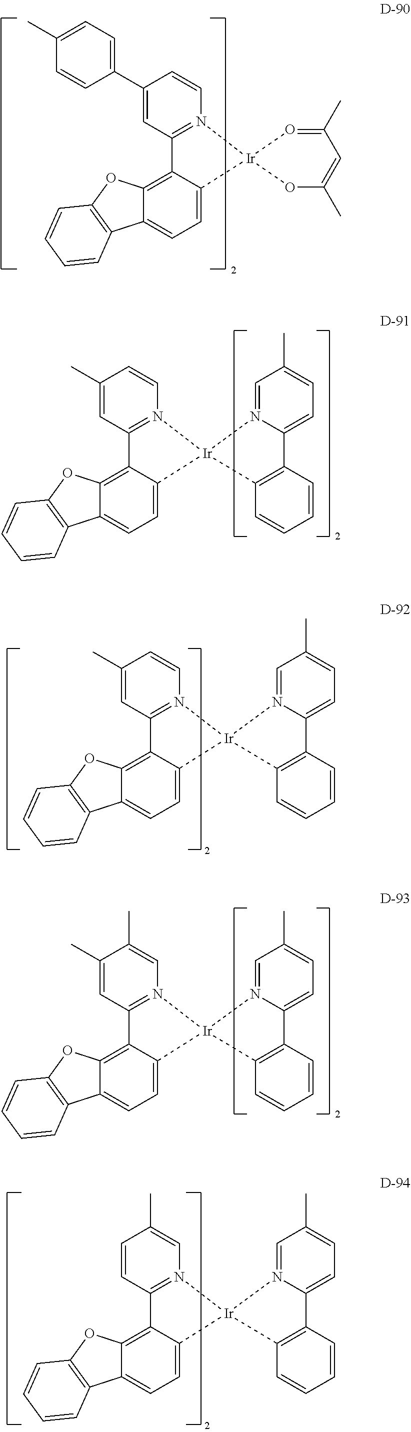

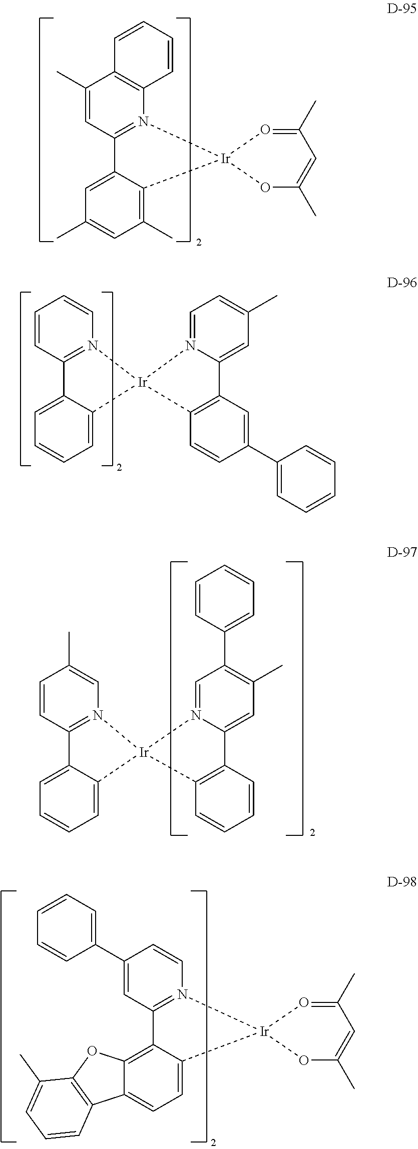

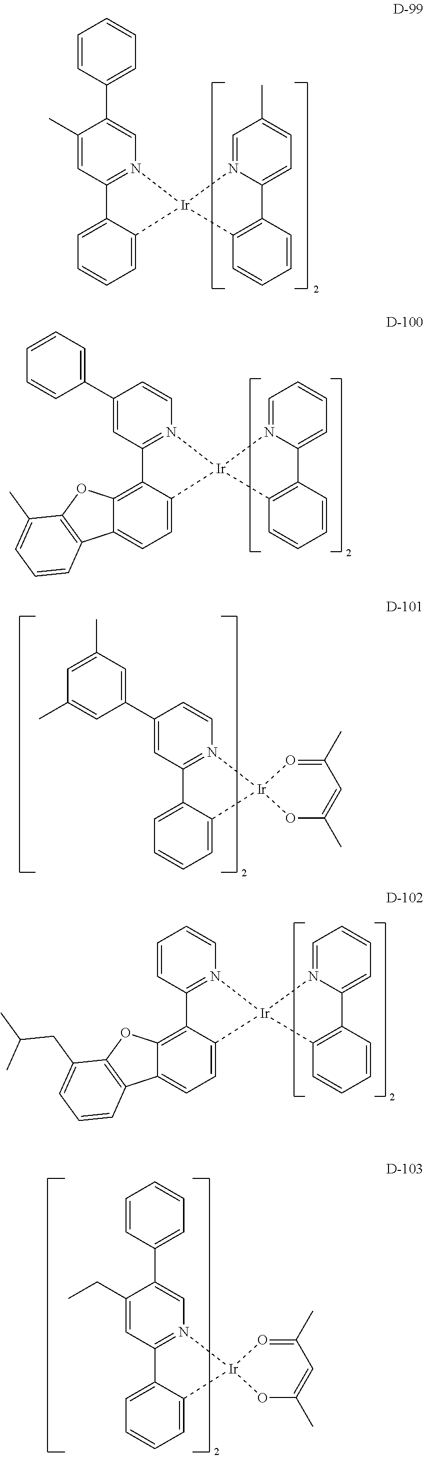

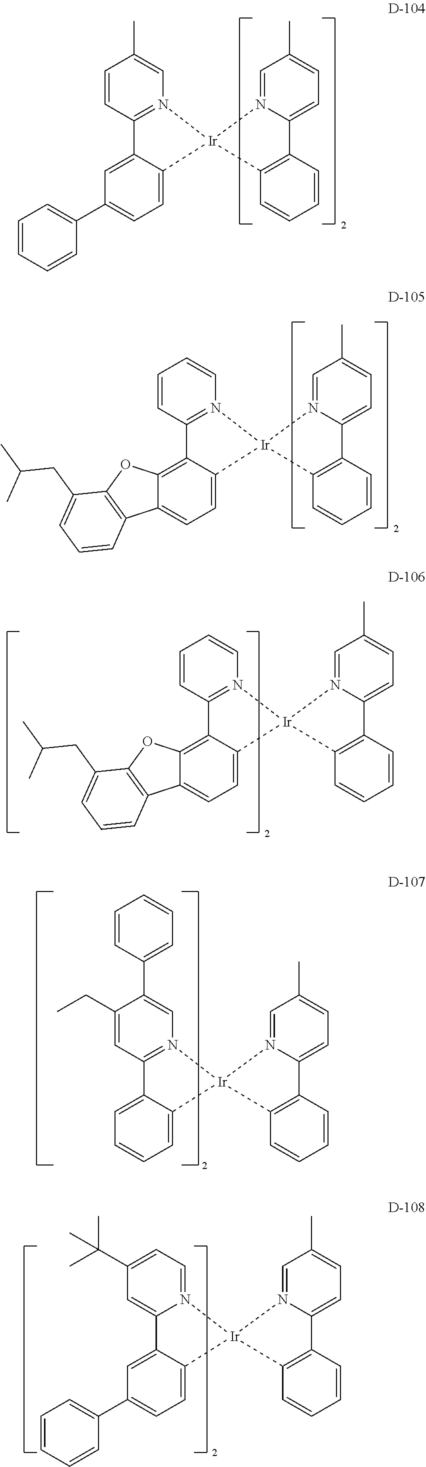

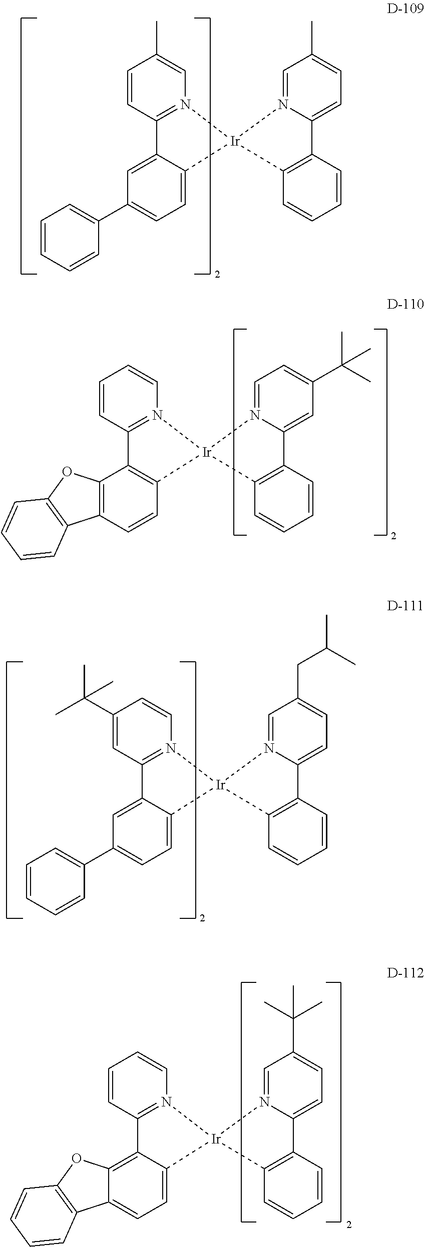

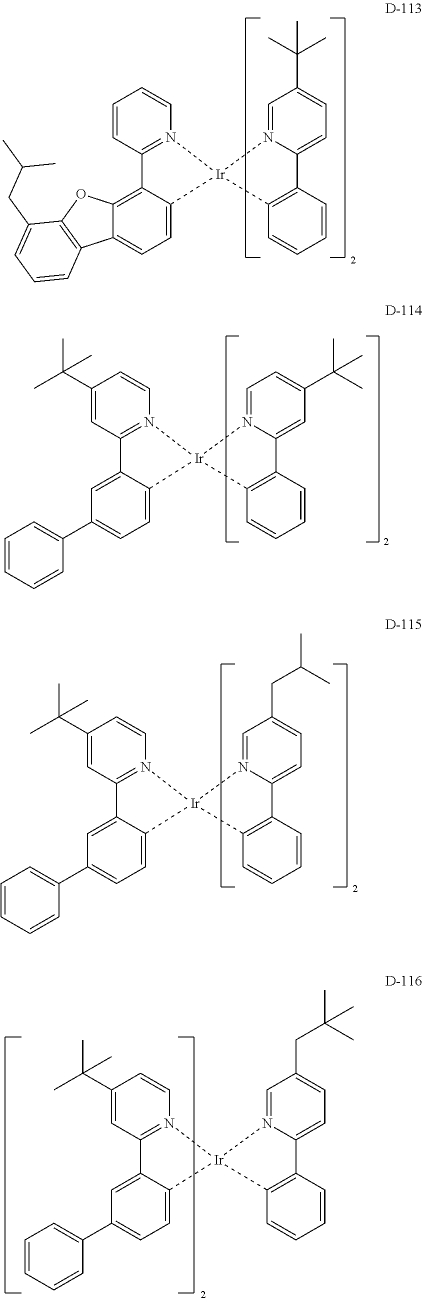

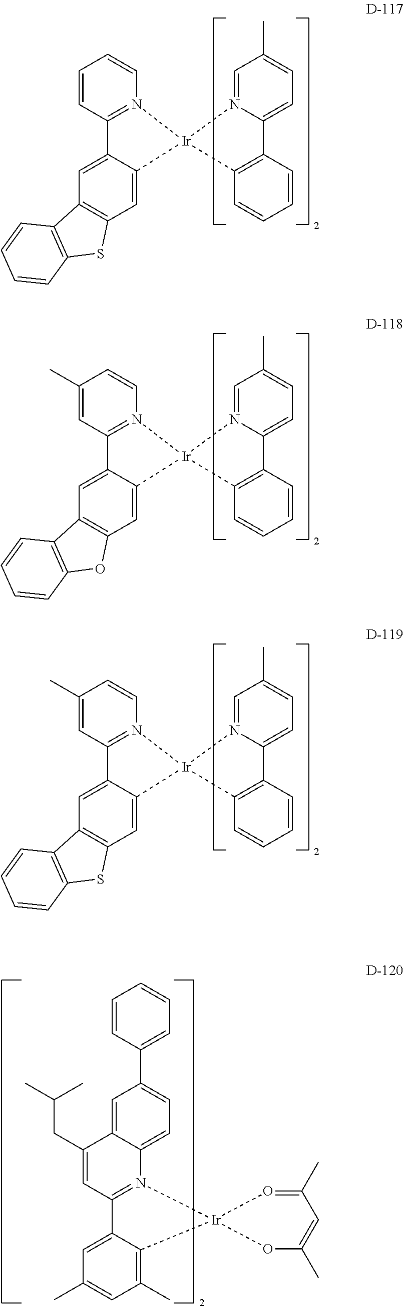

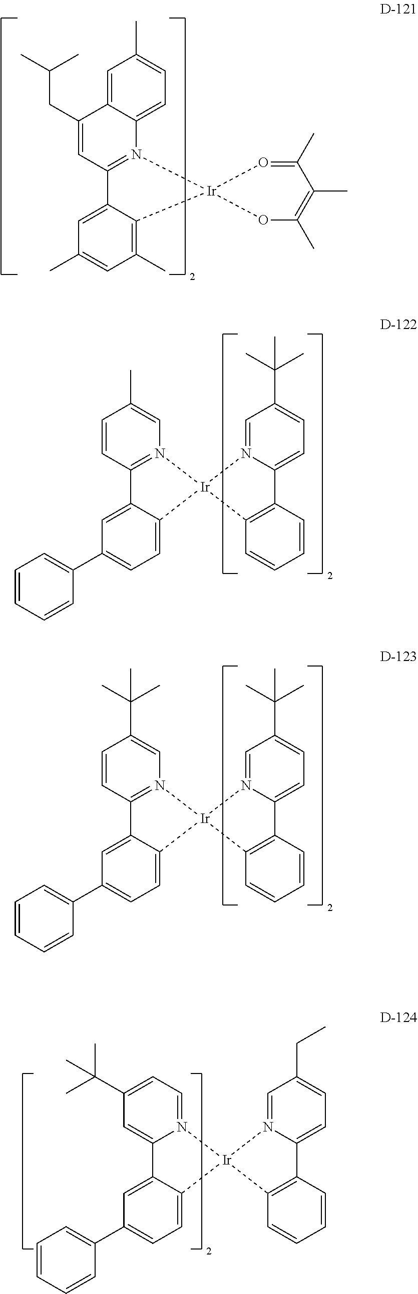

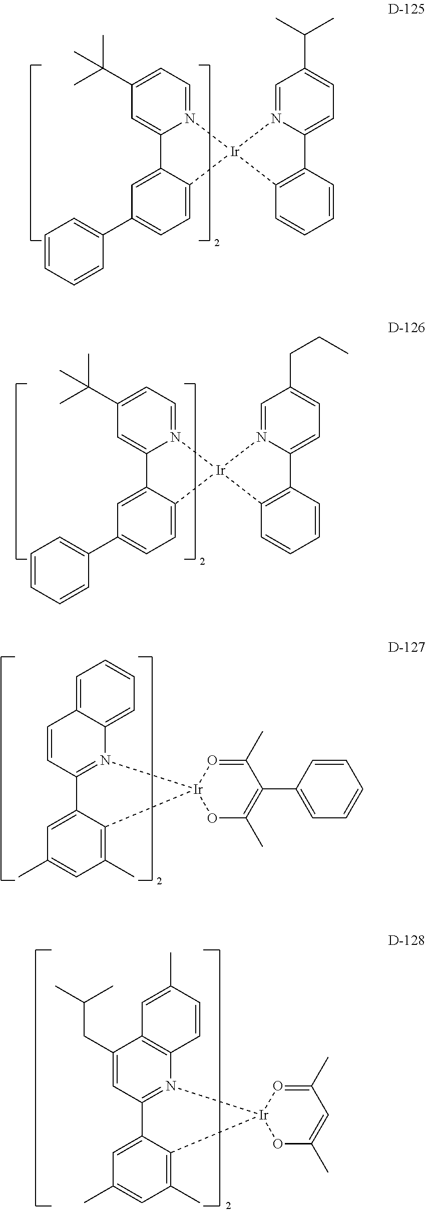

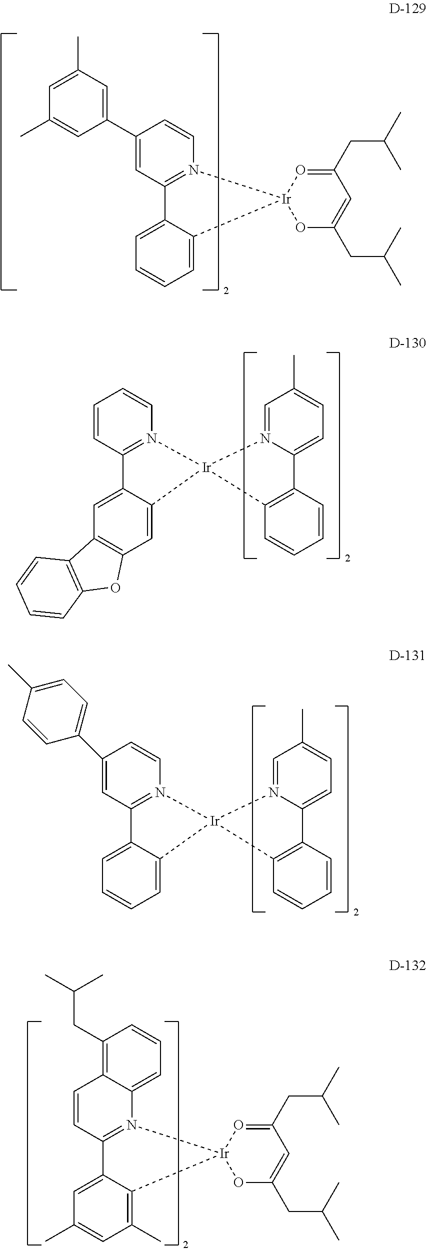

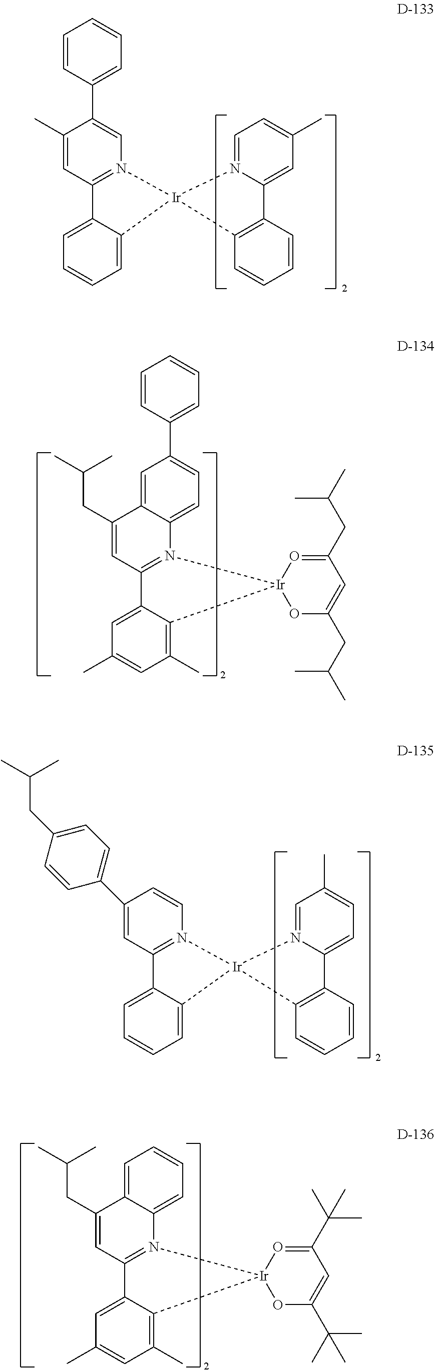

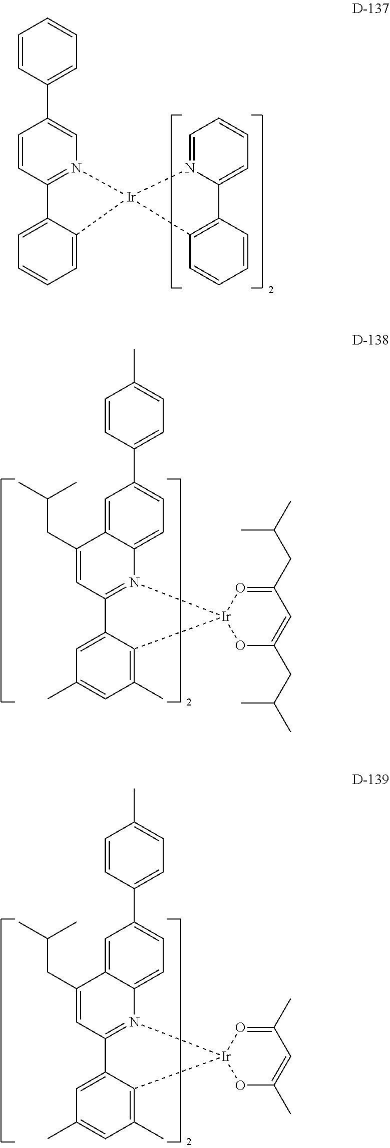

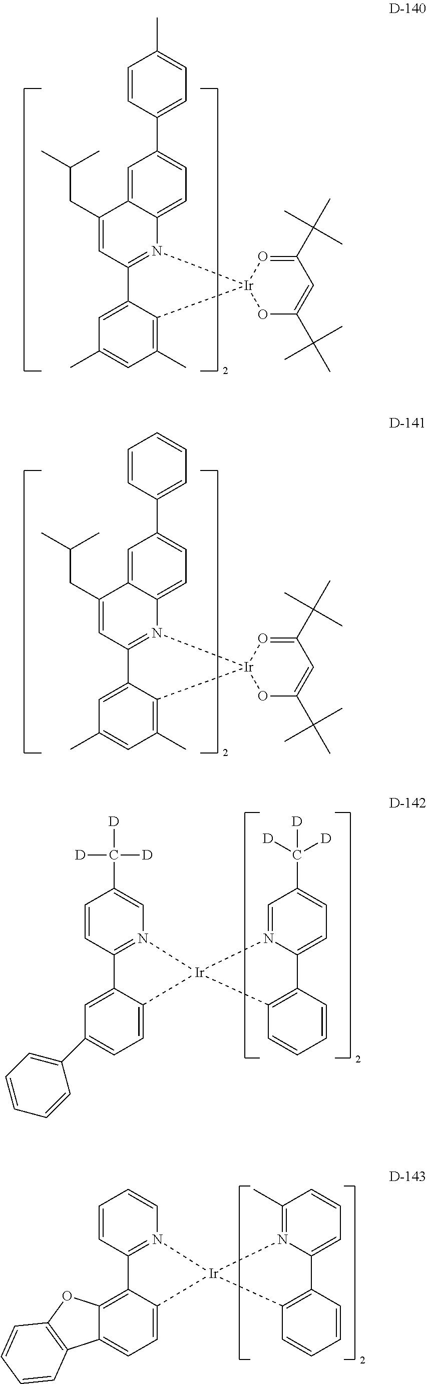

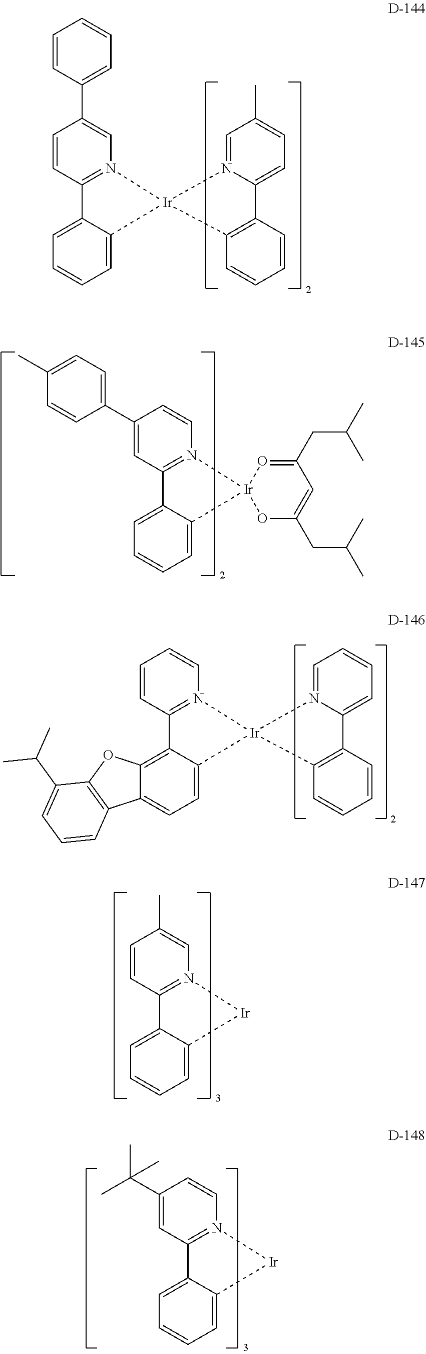

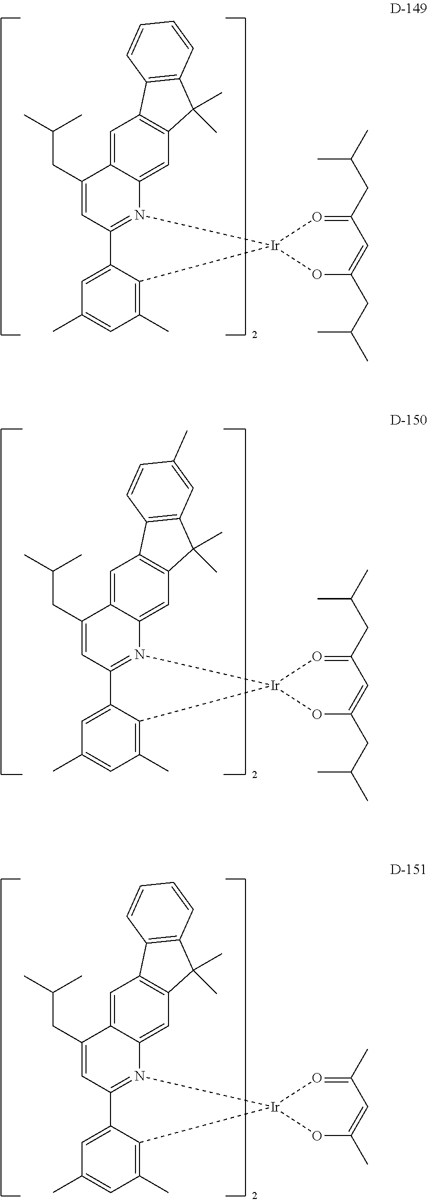

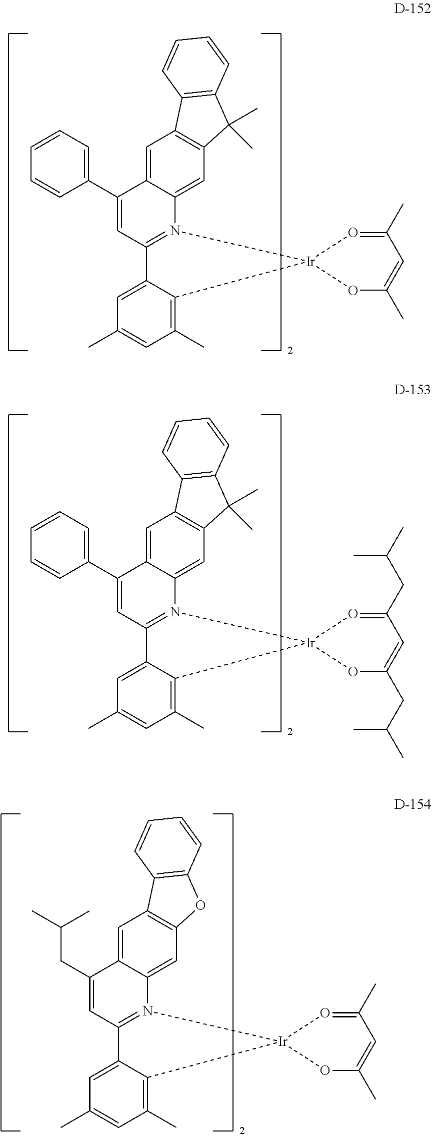

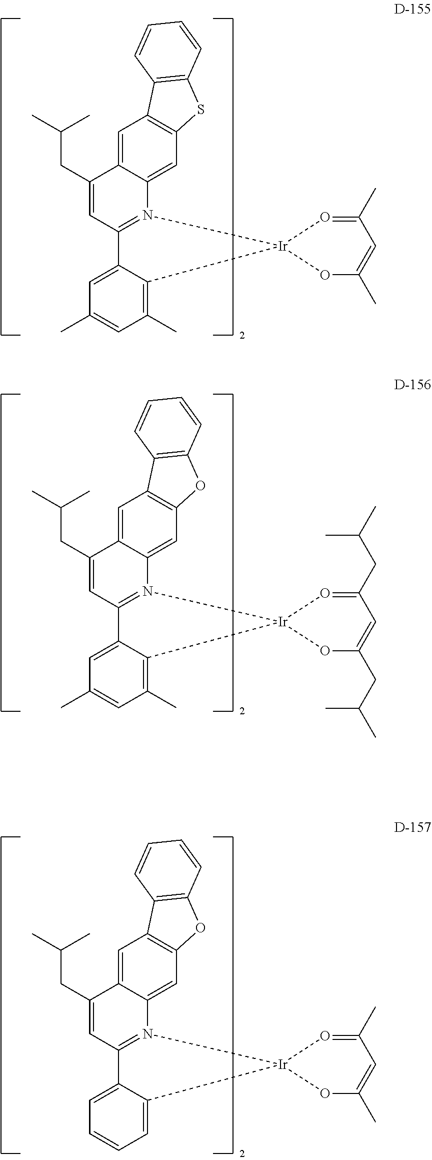

The organic electroluminescent device according to the present disclosure comprises a first electrode; a second electrode; and at least one organic layer between the first and second electrodes.

One of the first and second electrodes may be an anode, and the other may be a cathode. The organic layer may comprise a light-emitting layer, and may further comprise at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer.

The organic layer may further comprise at least one compound selected from the group consisting of arylamine-based compounds and styrylarylamine-based compounds.

In addition, in the organic electroluminescent device according to the present disclosure, the organic layer may further comprise at least one metal selected from the group consisting of metals of Group 1, metals of Group 2, transition metals of the 4^{th} period, transition metals of the 5^{th} period, lanthanides and organic metals of d-transition elements of the Periodic Table, or at least one complex compound comprising said metal.

In addition, the organic electroluminescent device according to the present disclosure may emit white light by further comprising at least one light-emitting layer which comprises a blue electroluminescent compound, a red electroluminescent compound or a green electroluminescent compound known in the field, besides the compound according to the present disclosure. Also, if necessary, a yellow or orange light-emitting layer can be further comprised in the device.

In the organic electroluminescent device according to the present disclosure, at least one layer (hereinafter, “a surface layer”) is preferably placed on an inner surface(s) of one or both electrode(s); selected from a chalcogenide layer, a metal halide layer, and a metal oxide layer. Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, said chalcogenide includes SiOₓ (1≤X≤2), AlOₓ (1≤X≤1.5), SiON, SiAlON, etc.; said metal halide includes LiF, MgF₂, CaF₂, a rare earth metal fluoride, etc.; and said metal oxide includes Cs₂O, Li₂O, MgO, SrO, BaO, CaO, etc.

A hole injection layer, a hole transport layer, an electron blocking layer, or a combination thereof can be used between the anode and the light-emitting layer. The hole injection layer may be multilayers in order to lower the hole injection barrier (or hole injection voltage) from the anode to the hole transport layer or the electron blocking layer, wherein each of the multilayers may use two compounds simultaneously. The hole transport layer or the electron blocking layer may also be multilayers.

An electron buffer layer, a hole blocking layer, an electron transport layer, an electron injection layer, or a combination thereof can be used between the light-emitting layer and the cathode. The electron buffer layer may be multilayers in order to control the injection of the electron and improve the interfacial properties between the light-emitting layer and the electron injection layer, wherein each of the multilayers may use two compounds simultaneously. The hole blocking layer or the electron transport layer may also be multilayers, wherein each of the multilayers may use a plurality of compounds.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or hole transport, or for preventing the overflow of electrons. When the light-emitting auxiliary layer is placed between the cathode and the light-emitting layer, it can be used for promoting the electron injection and/or electron transport, or for preventing the overflow of holes. Also, the hole auxiliary layer may be placed between the hole transport layer (or hole injection layer) and the light-emitting layer, and may be effective to promote or block the hole transport rate (or hole injection rate), thereby enabling the charge balance to be controlled. Further, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. When an organic electroluminescent device includes two or more hole transport layers, the hole transport layer, which is further included, may be used as a hole auxiliary layer or an electron blocking layer. The hole auxiliary layer and the electron blocking layer may have an effect of improving the efficiency and/or the lifespan of the organic electroluminescent device.

Preferably, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferably, the oxidative dopant includes various Lewis acids and acceptor compounds; and the reductive dopant includes alkali metals, alkali metal compounds, alkaline earth metals, rare-earth metals, and mixtures thereof. The reductive dopant layer may be employed as a charge-generating layer to prepare an organic EL device having two or more light-emitting layers which emits white light.

In order to form each layer constituting the organic electroluminescent device of the present disclosure, dry film-forming methods such as vacuum deposition, sputtering, plasma, ion plating methods, etc., or wet film-forming methods such as spin coating, dip coating, flow coating methods, etc., can be used. When forming the film of the first and second host compounds of the present disclosure, a co-evaporation or a mixed evaporation method is used.

When using a wet film-forming method, a thin film is formed by dissolving or dispersing the material constituting each layer in suitable solvents, such as ethanol, chloroform, tetrahydrofuran, dioxane, etc. The solvents are not particularly limited as long as the material constituting each layer is soluble or dispersible in the solvents, which do not cause any problems in forming a film.

By using the organic electroluminescent device of the present disclosure, a display device, for example, for smartphones, tablets, notebooks, PCs, TVs, or vehicles, or a lighting device, for example, an indoor or outdoor lighting device, can be produced.

Hereinafter, the preparation method of the compounds of the present disclosure, the physical properties of the compounds, and the luminous properties of the organic electroluminescent device comprising the compounds will be explained in detail with reference to the representative compounds of the present disclosure. However, the present disclosure is not limited to the Examples below.

### EXAMPLE 1: PREPARATION OF COMPOUND C-2

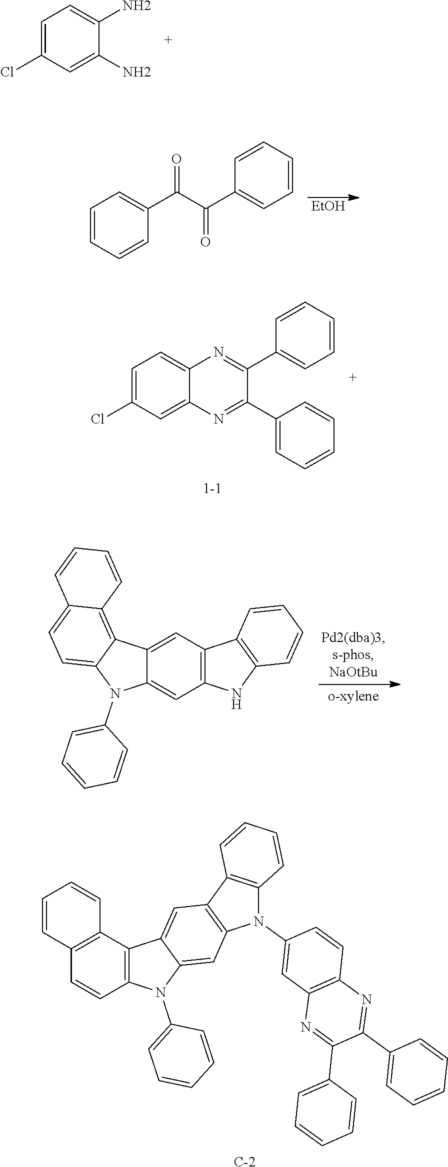

### Synthesis of Compound 1-1

17 g of 4-chlorobenzene-1,2-diamine (142 mmol) and 30 g of benzyl (119 mmol) were dissolved in 600 mL of ethanol in a flask, and the mixture was stirred at 110° C. for 4 hours. After completion of the reaction, the obtained solid was filtered, dried, and separated by column chromatography to obtain 20 g of compound 1-1 (yield: 53%).

### Synthesis of Compound C-2

6.95 g of compound 1-1 (21.0 mmol), 7 g of 7-phenyl-7,9-dihydrobenzo[g]indolo[2,3-b]carbazole (19.3 mmol), 833 mg of Pd₂(dba)₃ (0.915 mmol), 751 mg of 2-dichlorohexylphosphino-2′,6′-dimethoxybiphenyl (s-Phos) (1.83 mmol), and 5.27 g of NaOtBu (54.9 mmol) were dissolved in 100 mL of o-xylene in a flask, and the mixture was refluxed at 180° C. for 2 hours. After completion of the reaction, the reactant was filtered with celite, dried, and separated by column chromatography to obtain 8.2 g of compound C-2 (yield: 67.7%).

¹H NMR (600 MHz, DMSO, δ) 9.364 (s, 1H), 8.437-8.433 (d, J=24 Hz, 1H), 8.423-8.344 (m, 3H), 8.047-8.029 (m, 2H) 7.850-7.830 (m, 2H), 7.598-7.531 (m, 11H), 7.490 (s, 1H), 7.455-7.360 (m, 8H)

| Compound | MW | M.P. |
|---|---|---|
| C-2 | 662.80 | 303° C. |

### EXAMPLE 2: PREPARATION OF COMPOUND C-62

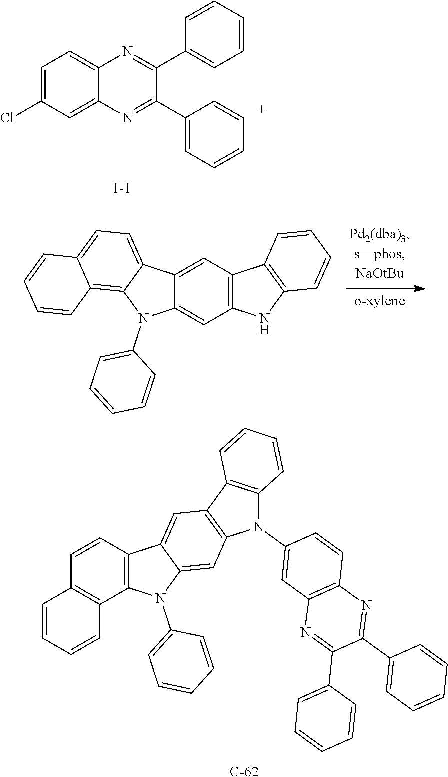

6.9 g of compound 1-1 (25.3 mmol), 7 g of 14-phenyl-12,14-dihydrobenzo[a]indolo[3,2-h]carbazole (21.1 mmol), 960 mg of Pd₂(dba)₃ (1.055 mmol), 866 mg of s-Phos (2.11 mmol), and 6 g of NaOtBu (63.3 mmol) were dissolved in 100 mL of o-xylene in a flask, and the mixture was refluxed at 180° C. for 2 hours. After completion of the reaction, the reactant was filtered with celite, dried, and separated by column chromatography to obtain 11 g of compound C-62 (yield: 85%).

¹H NMR (600 MHz, DMSO, δ) 8.895 (s, 1H), 8.375-8.361 (m, 2H), 8.303-8.285 (m, 2H), 7.971-7.949 (m, 2H) 7.771-7.757 (d, J=84 Hz, 1H), 7.572-7.504 (m, 10H), 7.390-7.305 (m, 10H), 7.135-7.134 (d, J=6 Hz, 2H)

| Compound | MW | M.P. |
|---|---|---|
| C-62 | 662.80 | 245° C. |

### EXAMPLE 3: PREPARATION OF COMPOUND C-32

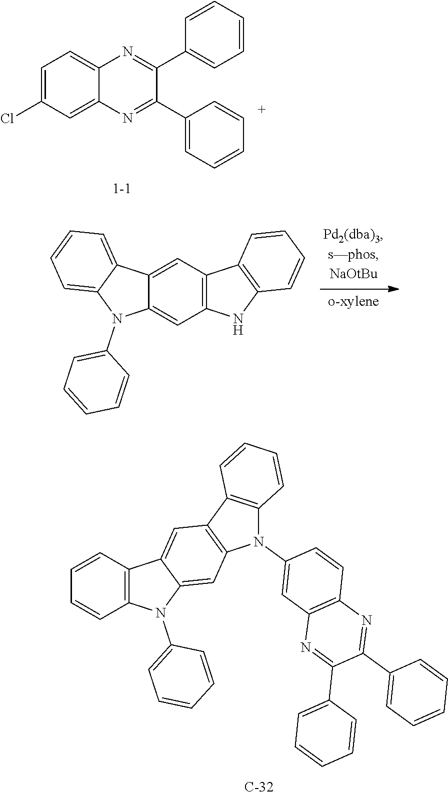

8.9 g of compound 1-1 (25.3 mmol), 7 g of 5-phenyl-5,7-dihydroindolo[2,3-b]carbazole (21.1 mmol), 960 mg of Pd₂(dba)₃ (1.055 mmol), 866 mg of s-Phos (2.11 mmol), and 6 g of NaOtBu (63.3 mmol) were dissolved in 100 mL of o-xylene in a flask, and the mixture was refluxed at 180° C. for 2 hours. After completion of the reaction, the reactant was filtered with celite, dried, and separated by column chromatography to obtain 11 g of compound C-32 (yield: 85%).

1H NMR (600 MHz, DMSO, δ) 8.865 (s, 1H), 8.403 (s, 1H), 8.345-8.330 (d, J=90 Hz, 1H), 8.288-8.263 (m, 2H) 8.010-7.992 (m, 1H), 7.577-7.522 (m, 9H), 7.490 (s, 1H), 7.423-7.352 (m, 13H)

| Compound | MW | M.P. |
|---|---|---|
| C-32 | 612.74 | 224° C. |

### EXAMPLE 4: PREPARATION OF COMPOUND C-182

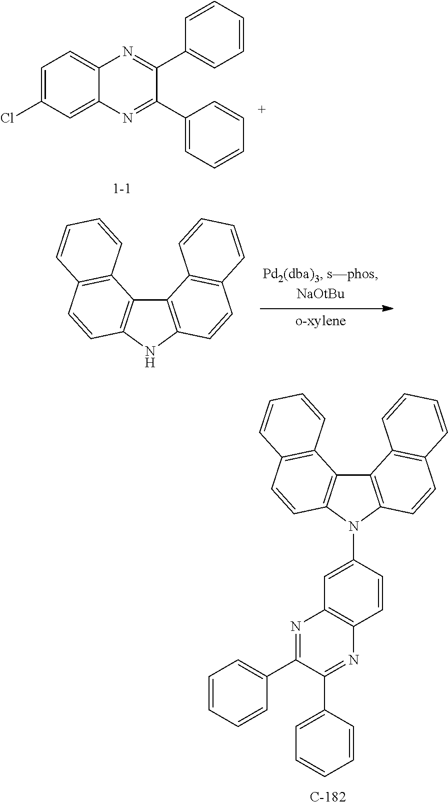 [SMILES: Clc1ccc2nc(-c3ccccc3)c(-c3ccccc3)nc2c1 ; c1ccc2c(c1)ccc1[nH]c3ccc4ccccc4c3c12 ; c1ccc(-c2nc3ccc(-n4c5ccc6ccccc6c5c5c6ccccc6ccc54)cc3nc2-c2ccccc2)cc1]

6 g of compound 1-1 (18.9 mmol), 4.2 g of 7H-dibenzo[c,g]carbazole (15.8 mmol), 718 mg of Pd₂(dba)₃ (0.789 mmol), 657 mg of s-Phos (1.6 mmol), and 4.5 g of NaOtBu (47.34 mmol) were dissolved in 100 mL of o-xylene in a flask, and the mixture was refluxed at 180° C. for 2 hours. After completion of the reaction, the reactant was filtered with celite, dried, and separated by column chromatography to obtain 4.5 g of compound C-182 (yield: 85%).

¹H NMR (600 MHz, DMSO, δ) 9.279-9.260 (d, 2H), 8.467-8.441 (m, 2H), 8.061-8.049 (d, 2H), 8.009-8.005 (s, J=24 Hz, 1H) 7.994-7.991 (d, 2H), 7.884-7.710 (m, 4H), 7.600-7.542 (m, 6H), 7.417-7.347 (m, 6H)

| Compound | MW | M.P. |
|---|---|---|
| C-182 | 547.66 | 278° C. |

### EXAMPLE 5: PREPARATION OF COMPOUND C-152

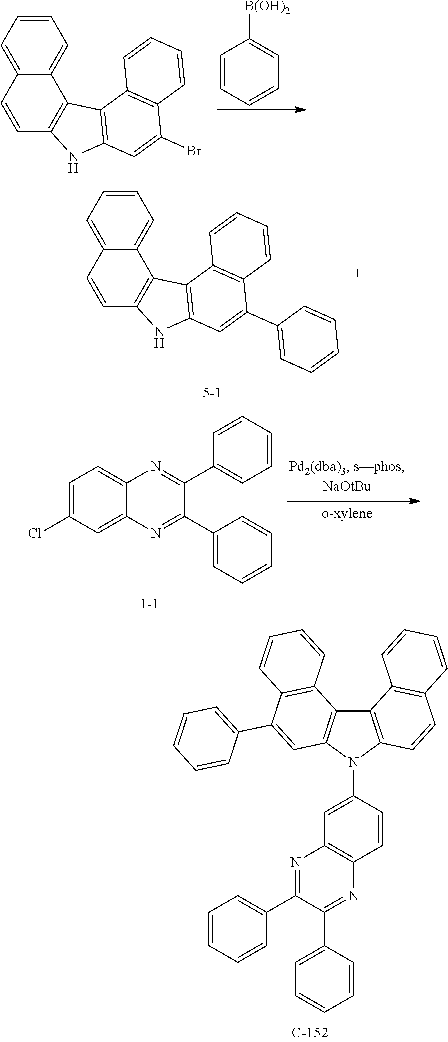

### Synthesis of Compound 5-1

20 g of 5-bromo-7H-dibenzo[c,g]carbazole (57.76 mmol), 8.4 g of phenylboronic acid (69.32 mmol), 3.3 g of Pd(PPh₃)₄ (2.88 mmol), 16 g of K₂CO₃ (115.5 mmol), 231 mL of toluene, 58 mL of ethanol, and 58 mL of purified water were introduced into a flask, and the mixture was stirred under reflux for a day. After completion of the reaction, the mixture was cooled to room temperature, and extracted with distilled water and ethyl acetate (EA). The obtained solid was dissolved in methylene chloride (MC) and separated by column chromatography to obtain 8 g of compound 5-1 (yield: 40%).

### Synthesis of Compound C-152

5.5 g of compound 1-1 (17.5 mmol), 5 g of compound 5-1 (14.6 mmol), 664 mg of Pd₂(dba)₃ (0.73 mmol), 600 mg of s-Phos (1.46 mmol), and 4.2 g of NaOtBu (43.6 mmol) were dissolved in 100 mL of o-xylene in a flask, and the mixture was refluxed at 180° C. for 2 hours. After completion of the reaction, the reactant was filtered with celite, dried, and separated by column chromatography to obtain 3.1 g of compound C-152 (yield: 34%).

¹H NMR (600 MHz, DMSO, δ) 9.311-9.274 (d, 2H), 8.451-8.405 (m, 2H), 8.050-8.038 (m, 2H), 7.991-7.973 (s, J=18 Hz, 1H)) 7.884-7.869 (s, J=90 Hz, 1H), 7.725-7.3321 (m, 4H)

| Compound | MW | M.P. |
|---|---|---|
| C-152 | 623.76 | 231° C. |

### EXAMPLE 6: PREPARATION OF COMPOUND C-13

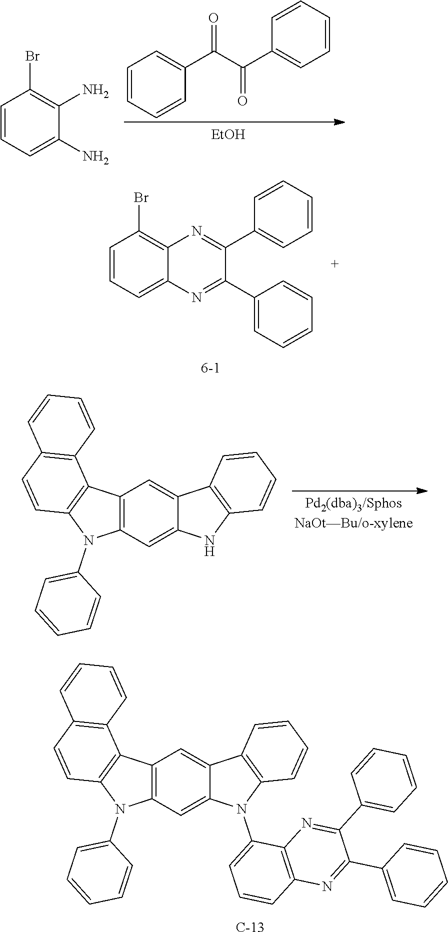

### Synthesis of Compound 6-1

50 g of 3-bromobenzene-1,2-diamine (267 mmol) and 67.5 g of benzyl (321 mmol) were dissolved in 1.3 L of ethanol in a flask, and the mixture was stirred under reflux for 2.5 hours. After completion of the reaction, the mixture was cooled to 0° C., and the obtained solid was filtered, dried, and separated with silica filter to obtain 72 g of compound 6-1 (yield: 75%).

### Synthesis of Compound C-13

8 g of compound 6-1 (22 mmol), 7 g of 7-phenyl-7,9-dihydrobenzo[g]indolo[2,3-b]carbazole (18 mmol), 0.83 g of Pd₂(dba)₃ (0.92 mmol), 0.75 g of s-Phos (1.8 mmol), and 5.3 g of NaOtBu (55 mmol) were dissolved in 100 mL of o-xylene in a flask, and the mixture was stirred under reflux for a day. After completion of the reaction, the reactant was cooled to room temperature, filtered with celite, and distilled under reduced pressure, and the resulting solid was separated by column chromatography to obtain 1.1 g of compound C-13 (yield: 9%).

¹H NMR (600 MHz, DMSO, δ) 9.66 (s, 1H), 9.26-9.25 (d, 1H), 8.68-8.67 (t, 1H), 8.34-8.32 (m, 1H) 8.23-8.22 (m, 1H), 8.13-8.09 (m, 2H), 7.95 (d, 1H), 7.88-7.86 (t, 1H), 7.59-7.56 (t, 3H), 7.51-7.38 (m, 10H), 7.25-7.24 (s, 1H), 7.17-7.14 (m, 1H), 7.07-7.02 (m, 4H), 7.00 (s, 1H)

| Compound | MW | M.P. |
|---|---|---|
| C-13 | 662.80 | 184° C. |

### EXAMPLE 7: PREPARATION OF COMPOUND C-193

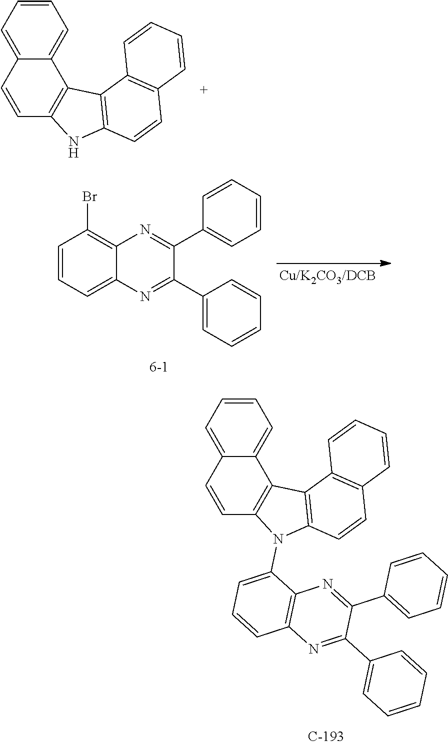 [SMILES: c1ccc2c(c1)ccc1[nH]c3ccc4ccccc4c3c12 ; Brc1cccc2nc(-c3ccccc3)c(-c3ccccc3)nc12 ; c1ccc(-c2nc3cccc(-n4c5ccc6ccccc6c5c5c6ccccc6ccc54)c3nc2-c2ccccc2)cc1]

5 g of 7H-dibenzo[c,g]carbazole (18 mmol), 8.1 g of compound 6-1 (22 mmol), 0.6 g of Cu (9 mmol), 5.1 g of K₂CO₃ (37 mmol), and 94 mL of dichlorobenzene (DCB) were introduced into a flask, and the mixture was stirred under reflux for a day. After completion of the reaction, the mixture was cooled to room temperature, and the obtained solid was filtered under reduced pressure. The obtained solid was dissolved in methylene chloride (MC) and separated by column chromatography to obtain 9.8 g of compound C-193 (yield: 95%).

¹H NMR (600 MHz, DMSO) 9.17-9.15 (d, 2H), 8.49-4.48 (d, 1H), 8.27-8.26 (d, 1H), 8.22-8.19 (t, 1H), 8.16-8.15 (d, 2H), 7.96-7.94 (d, 2H), 7.79-7.77 (t, 2H), 7.59-7.57 (t, 2H), 7.50-7.48 (m, 4H), 7.43-7.40 (m, 1H), 7.39-7.36 (m, 2H), 7.13-7.10 (m, 1H), 7.00-6.96 (m, 4H)

| Compound | MW | M.P. |
|---|---|---|
| C-193 | 547.65 | 278.3° C. |

### EXAMPLE 8: PREPARATION OF COMPOUND C-163

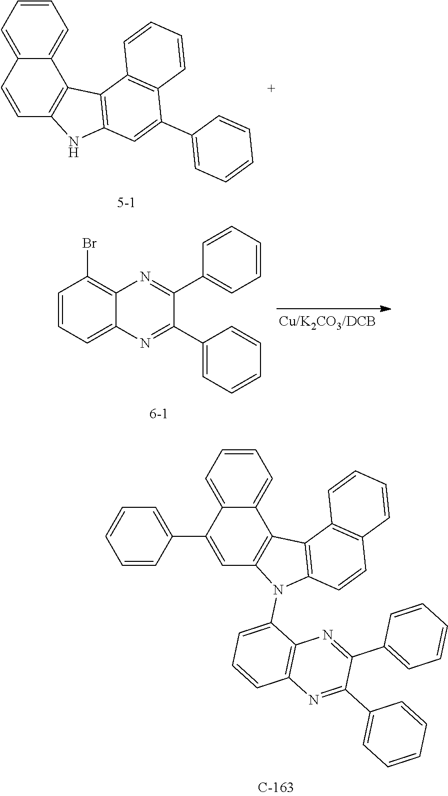

5 g of compound 5-1 (14.55 mmol), 6.3 g of compound 6-1 (17.47 mmol), 0.4 g of Cu (7.27 mmol), 4 g of K₂CO₃ (29.11 mmol), and 73 mL of DCB were introduced into a flask, and the mixture was stirred under reflux for a day. After completion of the reaction, the mixture was cooled to room temperature, and the obtained solid was filtered under reduced pressure. The obtained solid was dissolved in MC and separated by column chromatography to obtain 3.4 g of compound C-163 (yield: 38%).

¹H NMR (600 MHz, DMSO), 9.24-9.22 (d, 1H), 9.18-9.16 (d, 1H), 8.46-8.44 (d, 1H), 8.31-8.30 (d, 1H), 8.20-8.17 (t, 2H), 7.98-7.97 (d, 1H), 7.95-7.94 (d, 1H), 7.81-7.78 (m, 2H), 7.61-7.54 (t, 1H), 7.52-7.35 (m, 13H), 7.14-7.12 (m, 1H), 7.00-6.99 (d, 4H)

| Compound | MW | M.P. |
|---|---|---|
| C-163 | 623.7 | 255.8° C. |

### EXAMPLE 9: PREPARATION OF COMPOUND C-20

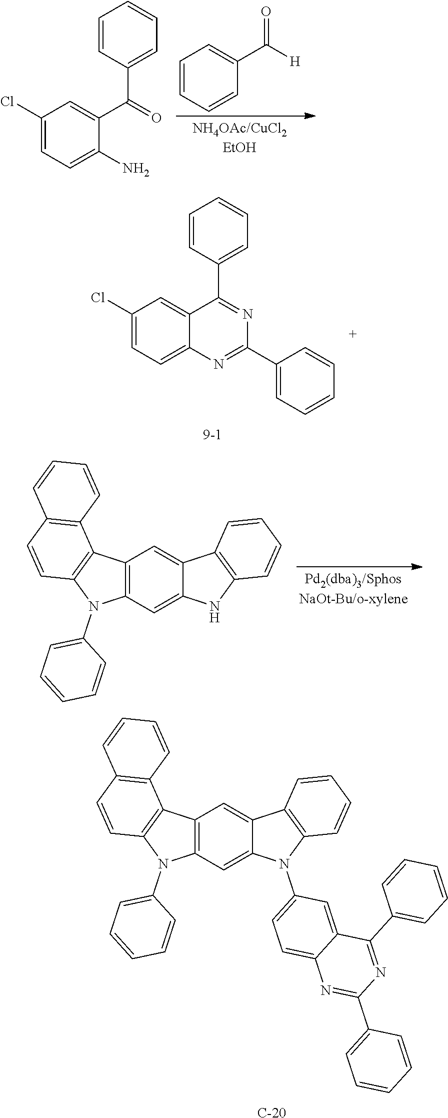

### Synthesis of Compound 9-1

24 g of (2-amino-5-chlorophenyl)(phenyl)methaneone (104 mmol), 12.1 g of benzaldehyde (114 mmol), 24 g of NH₄OAc (311 mmol), and 27.9 g of CuCl₂ (207 mmol) were dissolved in 1 L of ethanol in a flask, and the mixture was stirred under reflux for 3 hours. After completion of the reaction, the mixture was cooled to 0° C., and the solid obtained by adding water was filtered, dried, and separated with silica filter to obtain 28.0 g of compound 9-1 (yield: 85%).

### Synthesis of Compound C-20

5 g of compound 9-1 (16 mmol), 5 g of 7-phenyl-7,9-dihydrobenzo[g]indolo[2,3-b]carbazole (13 mmol), 0.60 g of Pd₂(dba)₃ (0.65 mmol), 0.54 g of s-Phos (1 mmol), and 3.8 g of NaOtBu (39 mmol) were dissolved in 70 mL of o-xylene in a flask, and the mixture was stirred under reflux for 1 hour. After completion of the reaction, the reactant was cooled to room temperature, filtered with celite, and distilled under reduced pressure, and the resulting solid was separated by column chromatography to obtain 2.4 g of compound C-20 (yield: 28%).

¹H NMR (600 MHz, DMSO, δ) 9.67 (s, 1H), 9.25-9.24 (d, 1H), 8.67-8.66 (m, 3H), 8.42 (s, 2H) 8.22 (s, 1H), 8.14-8.13 (d, 1H), 7.98-7.96 (d, 1H), 7.87-7.85 (t, 1H), 7.83-7.82 (d, 2H), 7.70-7.68 (m, 4H), 7.63-7.62 (m, 3H), 7.59-7.53 (m, 7H), 7.48-7.46 (t, 2H), 7.42-7.40 (m, 1H)

| Compound | MW | M.P. |
|---|---|---|
| C-20 | 662.80 | 190° C. |

### EXAMPLE 10: PREPARATION OF COMPOUND C-170

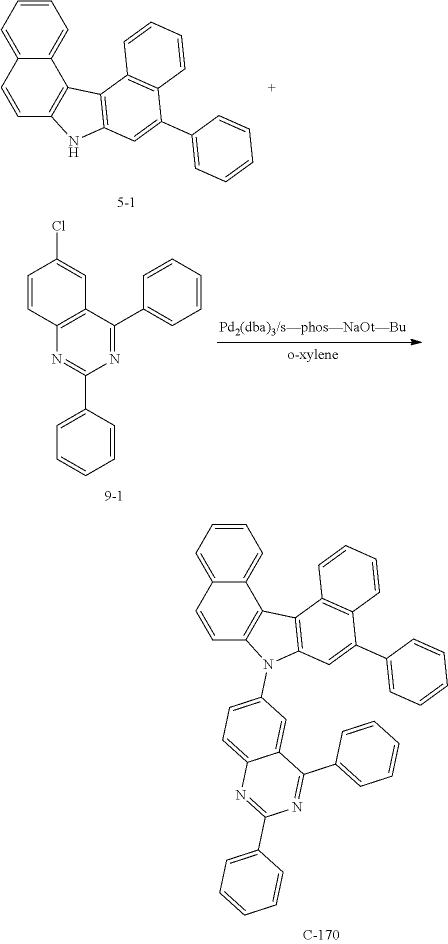 [SMILES: c1ccc(-c2cc3[nH]c4ccc5ccccc5c4c3c3ccccc23)cc1 ; Clc1ccc2nc(-c3ccccc3)nc(-c3ccccc3)c2c1 ; c1ccc(-c2nc(-c3ccccc3)c3cc(-n4c5ccc6ccccc6c5c5c6ccccc6c(-c6ccccc6)cc54)ccc3n2)cc1]

5 g of compound 5-1 (14.55 mmol), 5.5 g of compound 9-1 (17.47 mmol), 0.66 g of Pd₂(dba)₃ (0.72 mmol), 0.6 g of s-Phos (1.49 mmol), and 4.2 g of NaOtBu (43.67 mmol) were dissolved in 73 mL of o-xylene in a flask, and the mixture was stirred under reflux for a day. After completion of the reaction, the mixture was cooled to room temperature, and the obtained solid was filtered under reduced pressure. The obtained solid was dissolved in MC and separated by column chromatography to obtain 4.4 g of compound C-170 (yield: 49%).

¹H NMR (600 MHz, DMSO), 9.21-9.19 (d, 1H), 9.14-9.13 (d, 1H), 8.69-8.67 (m, 2H), 8.49-8.48 (d, 1H), 8.43-8.41 (m, 1H), 8.31-8.30 (d, 1H), 8.18-8.17 (d, 1H), 8.04-8.03 (d, 1H), 8.02-7.98 (m, 3H), 7.81-7.78 (m, 3H), 7.68 (s, 1H), 7.66-7.58 (m, 4H), 7.57-7.54 (m, 8H), 7.49-7.47 (m, 1H)

| Compound | MW | M.P. |
|---|---|---|
| C-170 | 623.7 | 241.9° C. |

### EXAMPLE 11: PREPARATION OF COMPOUND C-200

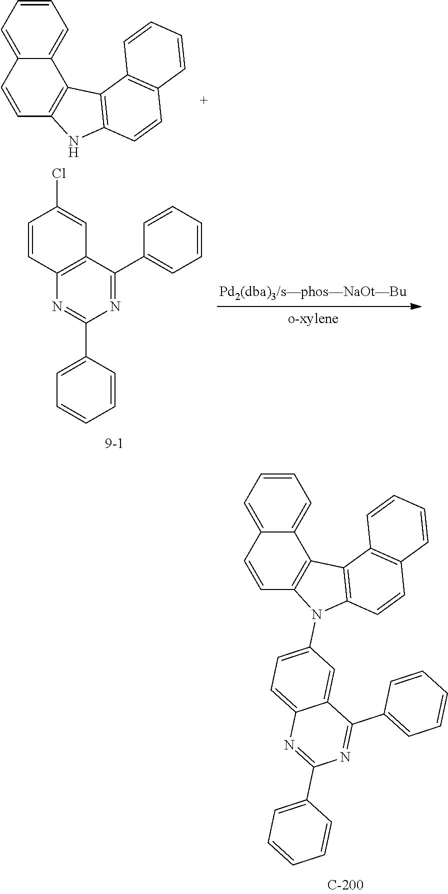

4 g of 7H-dibenzo[c,g]carbazole (14.96 mmol), 5.6 g of compound 9-1 (17.95 mmol), 0.68 g of Pd₂(dba)₃ (0.74 mmol), 0.6 g of s-Phos (1.49 mmol), and 4.31 g of NaOtBu (44.88 mmol) were dissolved in 75 mL of o-xylene in a flask, and the mixture was stirred under reflux for a day. After completion of the reaction, the mixture was cooled to room temperature, and the obtained solid was filtered under reduced pressure. The obtained solid was dissolved in MC and separated by column chromatography to obtain 4.2 g of compound C-200 (yield: 51%).

¹H NMR (600 MHz, DMSO), 9.13-9.12 (d, 2H), 8.72-8.70 (m, 2H), 8.52-8.50 (d, 1H), 8.38-8.36 (m, 1H), 8.29-8.28 (d, 1H), 8.17-8.15 (d, 2H), 8.04-8.01 (m, 4H), 7.79-7.75 (m, 4H), 7.67-7.62 (m, 3H), 7.61-7.56 (m, 5H)

| Compound | MW | M.P. |
|---|---|---|
| C-200 | 547.65 | 266.6° C. |

### EXAMPLE 12: PREPARATION OF COMPOUND C-43

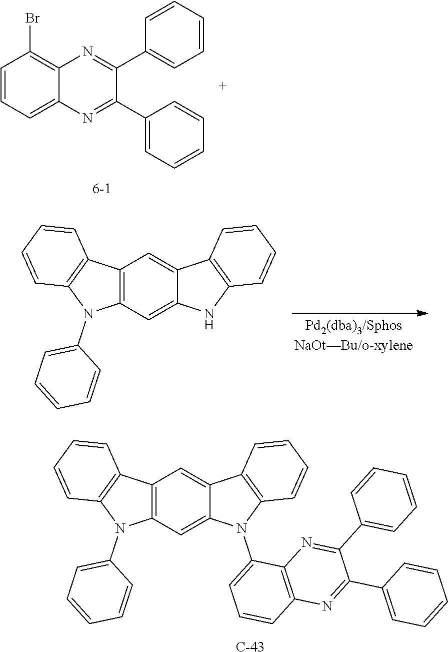

9.1 g of compound 6-1 (25 mmol), 7.0 g of 5-phenyl-5,7-dihydroindolo[2,3-b]carbazole (21 mmol), 0.96 g of Pd₂(dba)₃ (1 mmol), 0.86 g of s-Phos (2 mmol), and 6.1 g of NaOtBu (63 mmol) were dissolved in 100 mL of o-xylene in a flask, and the mixture was stirred under reflux for a day. After completion of the reaction, the mixture was cooled to room temperature, filtered with celite, and distilled under reduced pressure, and the resulting solid was separated by column chromatography to obtain 1.2 g of compound C-43 (yield: 9%).

¹H NMR (600 MHz, CDCl₃, δ) 9.13 (s, 1H), 8.38-8.35 (t, 2H), 8.34-8.32 (dd, 1H), 8.11-8.08 (t, 1H), 7.53-7.46 (m, 6H), 7.43-7.31 (m, 8H), 7.29-7.28 (d, 1H), 7.22-7.20 (d, 1H), 7.18-7.16 (t, 1H), 7.08-7.02 (m, 4H), 6.91 (s, 1H)

| Compound | MW | M.P. |
|---|---|---|
| C-43 | 612.74 | 239° C. |

### EXAMPLE 13: PREPARATION OF COMPOUND C-50

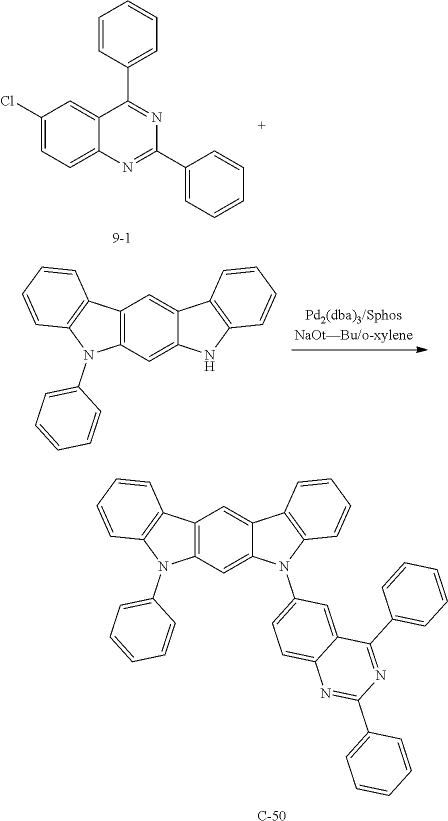

5.7 g of compound 9-1 (15 mmol), 5.0 g of 5-phenyl-5,7-dihydroindolo[2,3-b]carbazole (18 mmol), 0.69 g of Pd₂(dba)₃ (0.75 mmol), 0.62 g of s-Phos (2 mmol), and 4.3 g of NaOtBu (45 mmol) were dissolved in 70 mL of o-xylene in a flask, and the mixture was stirred under reflux for 2 hours. After completion of the reaction, the mixture was cooled to room temperature, filtered with celite, and distilled under reduced pressure, and the resulting solid was separated by column chromatography to obtain 3.8 g of compound C-50 (yield: 41%).

¹H NMR (600 MHz, CDCl₃, δ) 9.15 (d, 1H), 8.67-8.65 (m, 2H), 8.42-8.39 (m, 2H), 8.37-8.35 (m, 2H), 8.19 (m, 1H), 7.83-7.81 (m, 2H), 7.66-7.61 (m, 7H), 7.58-7.51 (m, 4H), 7.49-7.46 (m, 1H), 7.45-7.41 (m, 2H), 7.38-7.33 (m, 4H)

| Compound | MW | M.P. |
|---|---|---|
| C-50 | 612.74 | 180° C. |

### EXAMPLE 14: PREPARATION OF COMPOUND C-1

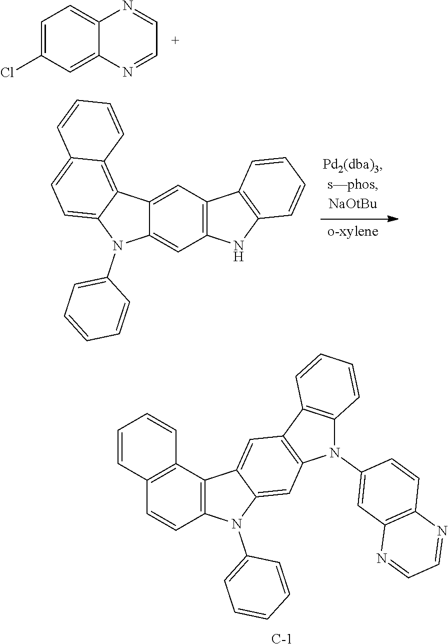

5 g of 6-chloroquinoxaline (30.04 mmol), 10 g of 14-phenyl-12,14-dihydrobenzo[a]indolo[3,2-h]carbazole (25.3 mmol), 1.2 g of Pd₂(dba)₃ (1.27 mmol), 1 g of s-Phos (2.53 mmol), and 7.3 g of NaOtBu (75.9 mmol) were dissolved in 130 mL of o-xylene in a flask, and the mixture was stirred at 180° C. for 2 hours. After completion of the reaction, the reactant was filtered with celite, dried, and separated by column chromatography to obtain 3.1 g of compound C-1 (yield: 24%).

1H NMR (600 MHz, CDCl₃, δ): 9.34 (s, 1H), 9.08 (d, J=8.3 Hz, 1H), 8.38 (dd, J=7.7, 1.1 Hz, 1H), 8.20 (dd, J=6.5, 3.2 Hz, 1H), 8.07-8.02 (m, 2H), 7.91 (dt, J=7.8, 1.4 Hz, 1H), 7.87-7.80 (m, 4H), 7.72 (t, J=7.8 Hz, 1H), 7.68-7.64 (m, 1H), 7.62-7.42 (m, 11H), 7.40-7.30 (m, 4H), 7.20-7.09 (m, 3H)

| Compound | MW | M.P. |
|---|---|---|
| C-1 | 510.6 | 258° C. |

### EXAMPLE 15: PREPARATION OF COMPOUND C-14

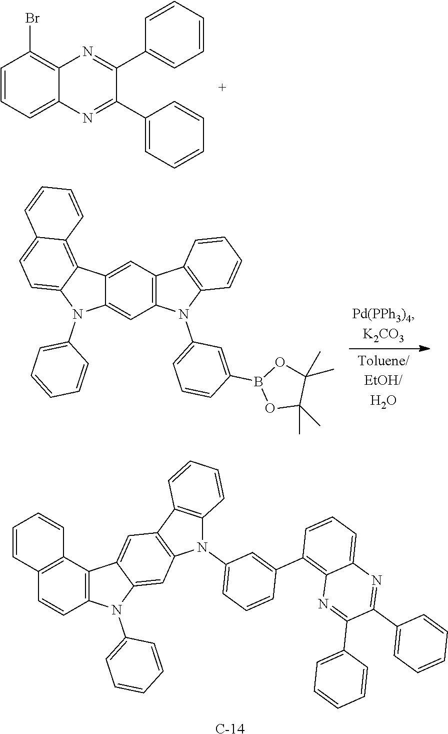

3.3 g of 5-bromo-2,3-diphenylquinoxaline (9.4 mmol), 7 g of 7-phenyl-9-(3-(4,4,5,5-tetramethyl-1,3,2-dioxaborolan-2-yl)phenyl)-7,9-dihydrobenzo[g]indolo[2,3-b]carbazole (21.1 mmol), 494 mg of Pd(PPh₃)₄ (0.43 mmol), and 3.5 g of K₂CO₃ (25.6 mmol) were dissolved in a mixture solvent of 50 mL of toluene, 25 mL of ethanol, and 25 mL of H₂O in a flask, and the mixture was stirred under reflux at 130° C. for 4 hours. After completion of the reaction, the reactant was filtered with celite, dried, and separated by column chromatography to obtain 3 g of compound C-14 (yield: 47.5%).

¹H NMR (600 MHz, CDCl₃, δ) 9.36 (d, J=0.8 Hz, 1H), 9.08 (d, J=8.3 Hz, 1H), 8.94-8.90 (m, 2H), 8.45-8.40 (m, 1H), 8.38 (d, J=2.3 Hz, 1H), 8.31 (d, J=8.8 Hz, 1H), 8.06 (ddd, J=13.7, 8.6, 1.6 Hz, 2H), 7.87-7.82 (m, 2H), 7.62-7.57 (m, 3H), 7.57-7.52 (m, 3H), 7.49-7.39 (m, 4H)

| Compound | MW | M.P. |
|---|---|---|
| C-14 | 738.89 | 271.3° C. |

### EXAMPLE 16: PREPARATION OF COMPOUND C-400

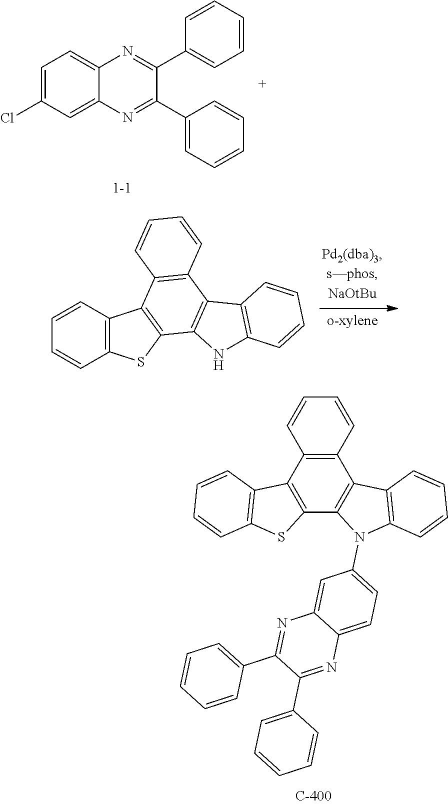 [SMILES: Clc1ccc2nc(-c3ccccc3)c(-c3ccccc3)nc2c1 ; c1ccc2c(c1)[nH]c1c3sc4ccccc4c3c3ccccc3c21 ; c1ccc(-c2nc3ccc(-n4c5ccccc5c5c6ccccc6c6c7ccccc7sc6c54)cc3nc2-c2ccccc2)cc1]

5.9 g of compound 1-1 (18.6 mmol), 5 g of 14H-benzo[c]benzo[4,5]thieno[2,3-a]carbazole (15.46 mmol), 704 mg of Pd₂(dba)₃ (0.773 mmol), 635 mg of s-Phos (1.546 mmol), and 4.5 g of NaOtBu (46.4 mmol) were dissolved in 130 mL of o-xylene in a flask, and the mixture was stirred under reflux at 180° C. for 3 hours. After completion of the reaction, the reactant was filtered with celite, dried, and separated by column chromatography to obtain 8.2 g of compound C-400 (yield: 87.8%).

¹H NMR (600 MHz, CDCl₃, δ) 9.23 (d, J=8.3 Hz, 1H), 9.11 (dd, J=8.2, 1.3 Hz, 1H), 8.93 (d, J=8.4 Hz, 1H), 8.75 (d, J=8.0 Hz, 1H), 8.51 (dd, J=2.3, 0.5 Hz, 1H), 8.45 (dd, J=8.6, 0.6 Hz, 1H), 7.92 (dd, J=8.7, 2.3 Hz, 1H), 7.84 (ddd, J=8.2, 6.9, 1.2 Hz, 1H), 7.78-7.72 (m, 2H), 7.68-7.64 (m, 2H), 7.63-7.59 (m, 2H), 7.53 (dddd, J=38.4, 8.1, 7.0, 1.2 Hz, 2H), 7.48-7.32 (m, 8H)

| Compound | MW | M.P. |
|---|---|---|
| C-400 | 603.74 | 290° C. |

### Comparative Example 1: Production of a Red Light-Emitting OLED not According to the Present Disclosure

An OLED not according to the present disclosure was produced. A transparent electrode indium tin oxide (ITO) thin film (10 Ω/sq) on a glass substrate for an OLED device (Geomatec, Japan) was subjected to an ultrasonic washing with acetone and isopropyl alcohol, sequentially, and was then stored in isopropanol. Next, the ITO substrate was mounted on a substrate holder of a vacuum vapor depositing apparatus. Compound HI-1 was introduced into a cell of said vacuum vapor depositing apparatus, and then the pressure in the chamber of said apparatus was controlled to 10⁻⁷ torr. Thereafter, an electric current was applied to the cell to evaporate the above-introduced material, thereby forming a first hole injection layer having a thickness of 80 nm on the ITO substrate. Compound HI-2 was then introduced into another cell of said vacuum vapor depositing apparatus, and was evaporated by applying an electric current to the cell, thereby forming a second hole injection layer having a thickness of 5 nm on the first hole injection layer. Compound HT-1 was introduced into another cell of said vacuum vapor depositing apparatus, and was evaporated by applying an electric current to the cell, thereby forming a first hole transport layer having a thickness of 10 nm on the second hole injection layer. Compound HT-2 was introduced into another cell of said vacuum vapor depositing apparatus, and was evaporated by applying an electric current to the cell, thereby forming a second hole transport layer having a thickness of 60 nm on the first hole transport layer. After forming the hole injection layers and the hole transport layers, a light-emitting layer was then deposited as follows. Compound X was introduced into one cell of the vacuum vapor depositing apparatus as a host, and compound D-71 was introduced into another cell as a dopant. The two materials were evaporated at different rates and were deposited in a doping amount of 3 wt % based on the total amount of the host and dopant to form a light-emitting layer having a thickness of 40 nm on the second hole transport layer. Next, compound ETL-1 and compound EIL-1 were evaporated in a weight ratio of 50:50 as electron transport materials to form an electron transport layer having a thickness of 35 nm on the light-emitting layer. After depositing Compound EIL-1 as an electron injection layer having a thickness of 2 nm on the electron transport layer, an Al cathode having a thickness of 80 nm was deposited by another vacuum vapor deposition apparatus on the electron injection layer. Thus, an OLED device was produced. All the materials used for producing the OLED device were purified by vacuum sublimation at 10⁻⁶ torr.

### Device Examples 1 to 9: Production of a Red Light-Emitting OLED According to the Present Disclosure

OLEDs were produced in the same manner as in Comparative Example 1, except that the compounds shown in Table 1 below were used as the light-emitting material instead of compound X.

The driving voltage, luminous efficiency, and CIE color coordinate at a luminance of 1,000 nit, and the time taken for the luminance to decrease from 100% to 90% at a luminance of 5,000 nit (lifespan; T90) of the OLEDs produced in Comparative Example 1 and Device Examples 1 to 9 are provided in Table 1 below.

TABLE 1

| | Light- | Driving | Luminous | Color | Color | |
|---|---|---|---|---|---|---|
| | emitting | voltage | efficiency | coordinate | coordinate | Lifespan |
| | layer | (V) | (cd/A) | (x) | (y) | (T90, hr) |
| Comparative | Compound X | 3.0 | 18.2 | 0.665 | 0.334 | 7.2 |
| Example 1 | | | | | | |
| Device | C-62 | 2.7 | 25.7 | 0.661 | 0.338 | 65.0 |
| Example 1 | | | | | | |
| Device | C-2 | 2.7 | 25.8 | 0.666 | 0.334 | 190.2 |
| Example 2 | | | | | | |
| Device | C-193 | 3.0 | 26.0 | 0.665 | 0.334 | 73.0 |
| Example 3 | | | | | | |
| Device | C-20 | 2.9 | 23.1 | 0.665 | 0.334 | 324.3 |
| Example 4 | | | | | | |
| Device | C-50 | 3.0 | 22.7 | 0.666 | 0.333 | 178.6 |
| Example 5 | | | | | | |
| Device | C-43 | 2.7 | 22.7 | 0.666 | 0.333 | 30.5 |
| Example 6 | | | | | | |
| Device | C-170 | 3.0 | 22.5 | 0.667 | 0.333 | 56.3 |
| Example 7 | | | | | | |
| Device | C-200 | 3.0 | 23.7 | 0.665 | 0.335 | 56.8 |
| Example 8 | | | | | | |
| Device | C-163 | 3.0 | 26.1 | 0.667 | 0.333 | 101.0 |
| Example 9 | | | | | | |

The organic electroluminescent device using the organic electroluminescent compound of the present disclosure as a host showed similar or lower driving voltage and higher efficiency than the organic electroluminescent device using the compound of the Comparative Example. Particularly, the lifespan characteristic was highly excellent.

### Device Examples 10 to 16: Production of a Red Light-Emitting OLED According to the Present Disclosure

In Device Examples 10 to 16, OLEDs were produced in the same manner as in Comparative Example 1, except that the first and second host compounds shown in Table 2 below as a host were introduced into two cells of the vacuum vapor deposition apparatus and compound D-71 as a dopant was introduced into another cell of the apparatus, the two host materials were evaporated at a rate of 1:1 and the dopant material was simultaneously deposited at a different rate in a doping amount of 3 wt %, based on the total weight of the host and dopant, to form a light-emitting layer having a thickness of 40 nm on the second hole transport layer.

The driving voltage, luminous efficiency, and CIE color coordinate at a luminance of 1,000 nit, and the time taken for the luminance to decrease from 100% to 90% at a luminance of 5,000 nit (lifespan; T90) of the OLEDs produced in Device Examples 10 to 16 are provided in Table 2 below.

TABLE 2

| | | | Driving | Luminous | Color | Color | |
|---|---|---|---|---|---|---|---|
| | First | Second | voltage | efficiency | coordinate | coordinate | Lifespan |
| | host | host | (V) | (cd/A) | (x) | (y) | (T90, hr) |
| Device | C-193 | H1-7 | 3.0 | 27.0 | 0.667 | 0.333 | 163.6 |
| Example 10 | | | | | | | |
| Device | C-182 | | 3.2 | 25.6 | 0.666 | 0.333 | 292.3 |
| Example 11 | | | | | | | |
| Device | C-43 | | 2.9 | 24.2 | 0.665 | 0.335 | 39.9 |
| Example 12 | | | | | | | |
| Device | C-50 | | 3.1 | 26.4 | 0.665 | 0.334 | 258.5 |
| Example 13 | | | | | | | |
| Device | C-163 | | 3.2 | 27.9 | 0.668 | 0.332 | 207.0 |
| Example 14 | | | | | | | |
| Device | C-163 | H1-1 | 3.2 | 29.3 | 0.666 | 0.334 | 244.8 |
| Example 15 | | | | | | | |
| Device | C-163 | H1-56 | 3.0 | 27.0 | 0.669 | 0.331 | 321.9 |
| Example 16 | | | | | | | |

From the properties of the OLEDs of Device Examples 10 to 16, it is verified that when using a combination of the first host material of the present disclosure with a second host material as a plurality of host materials, driving voltage is maintained at a similar level or lowered but the luminous efficiency and lifespan characteristic are highly enhanced.

TABLE 3
Organic electroluminescent materials used in the Device Examples and Comparative
Examples

| | |
|---|---|
| Hole Injection Layer/Hole Transport Layer | 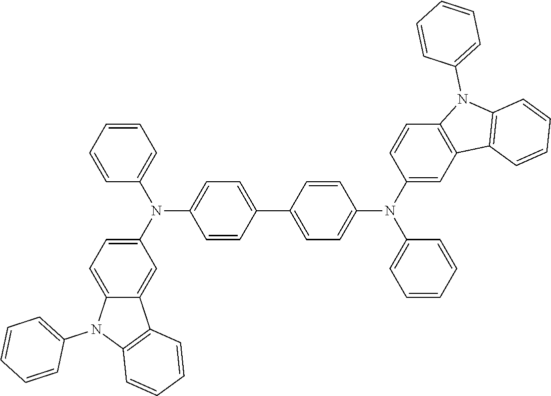 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1] |
| | HI-1 |
| | 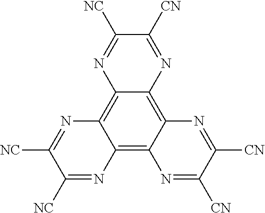 [SMILES: N#Cc1nc2c3nc(C#N)c(C#N)nc3c3nc(C#N)c(C#N)nc3c2nc1C#N] |
| | HI-2 |
| | 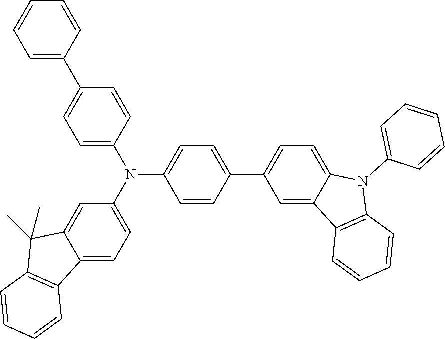 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] |
| | HT-1 |
| | 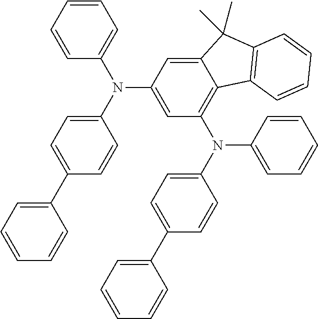 [SMILES: CC1(C)c2ccccc2-c2c(N(c3ccccc3)c3ccc(-c4ccccc4)cc3)cc(N(c3ccccc3)c3ccc(-c4ccccc4)cc3)cc21] |
| | HT-2 |
| Light-Emitting Layer | 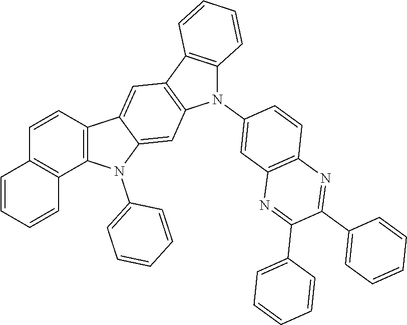 [SMILES: c1ccc(-c2nc3ccc(-n4c5ccccc5c5cc6c7ccc8ccccc8c7n(-c7ccccc7)c6cc54)cc3nc2-c2ccccc2)cc1] |
| | C-62 |
| | 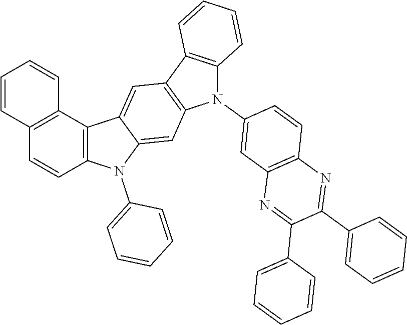 [SMILES: c1ccc(-c2nc3ccc(-n4c5ccccc5c5cc6c7c8ccccc8ccc7n(-c7ccccc7)c6cc54)cc3nc2-c2ccccc2)cc1] |
| | C-2 |
| | 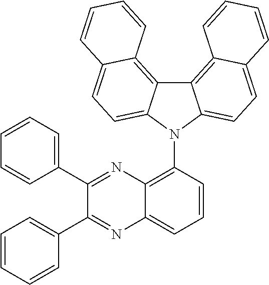 [SMILES: c1ccc(-c2nc3cccc(-n4c5ccc6ccccc6c5c5c6ccccc6ccc54)c3nc2-c2ccccc2)cc1] |
| | C-193 |
| | 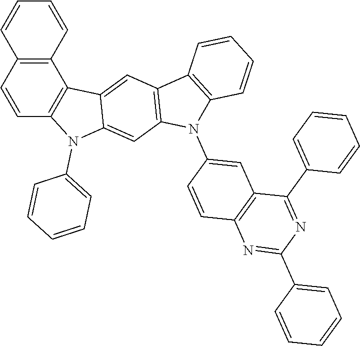 [SMILES: c1ccc(-c2nc(-c3ccccc3)c3cc(-n4c5ccccc5c5cc6c7c8ccccc8ccc7n(-c7ccccc7)c6cc54)ccc3n2)cc1] |
| | C-20 |
| | 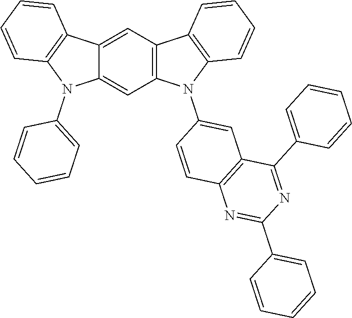 [SMILES: c1ccc(-c2nc(-c3ccccc3)c3cc(-n4c5ccccc5c5cc6c7ccccc7n(-c7ccccc7)c6cc54)ccc3n2)cc1] |
| | C-50 |
| | 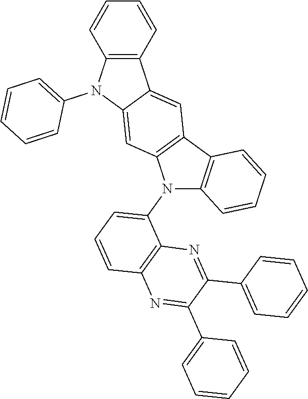 [SMILES: c1ccc(-c2nc3cccc(-n4c5ccccc5c5cc6c7ccccc7n(-c7ccccc7)c6cc54)c3nc2-c2ccccc2)cc1] |
| | C-43 |
| | 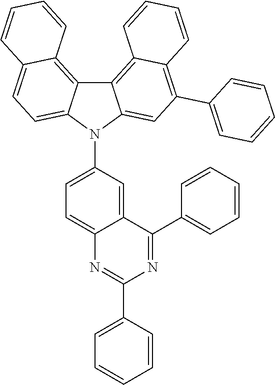 [SMILES: c1ccc(-c2nc(-c3ccccc3)c3cc(-n4c5ccc6ccccc6c5c5c6ccccc6c(-c6ccccc6)cc54)ccc3n2)cc1] |
| | C-170 |
| | 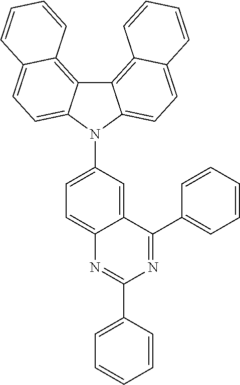 [SMILES: c1ccc(-c2nc(-c3ccccc3)c3cc(-n4c5ccc6ccccc6c5c5c6ccccc6ccc54)ccc3n2)cc1] |
| | C-200 |
| | 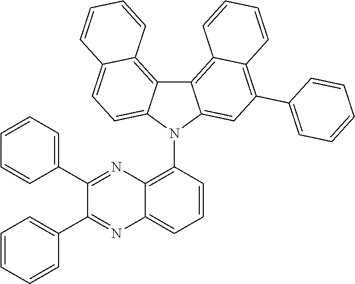 [SMILES: c1ccc(-c2nc3cccc(-n4c5ccc6ccccc6c5c5c6ccccc6c(-c6ccccc6)cc54)c3nc2-c2ccccc2)cc1] |
| | C-163 |
| | 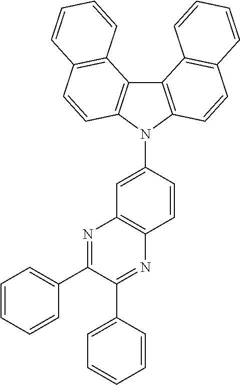 [SMILES: c1ccc(-c2nc3ccc(-n4c5ccc6ccccc6c5c5c6ccccc6ccc54)cc3nc2-c2ccccc2)cc1] |
| | C-182 |
| | 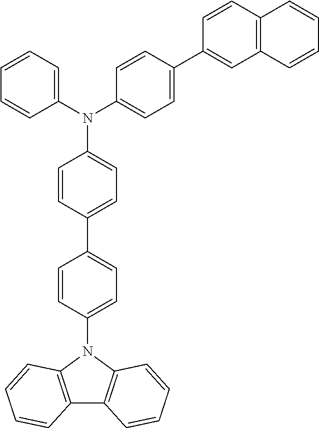 [SMILES: c1ccc(N(c2ccc(-c3ccc(-n4c5ccccc5c5ccccc54)cc3)cc2)c2ccc(-c3ccc4ccccc4c3)cc2)cc1] |
| | H1-7 |
| | 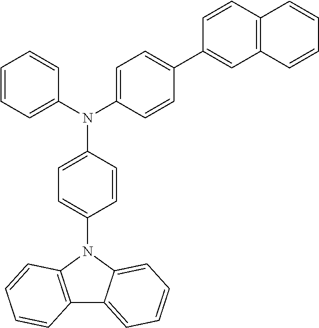 [SMILES: c1ccc(N(c2ccc(-c3ccc4ccccc4c3)cc2)c2ccc(-n3c4ccccc4c4ccccc43)cc2)cc1] |
| | H1-1 |
| | 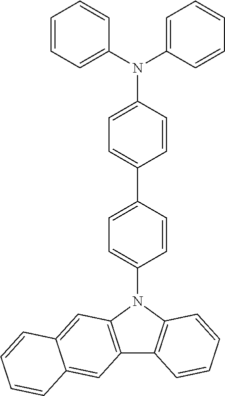 [SMILES: c1ccc(N(c2ccccc2)c2ccc(-c3ccc(-n4c5ccccc5c5cc6ccccc6cc54)cc3)cc2)cc1] |
| | H1-56 |
| | 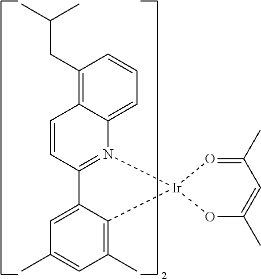 |
| | D-71 |
| | 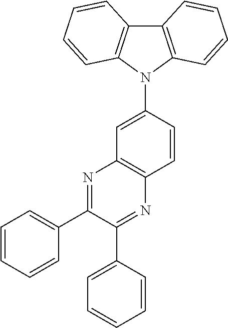 [SMILES: c1ccc(-c2nc3ccc(-n4c5ccccc5c5ccccc54)cc3nc2-c2ccccc2)cc1] |
| | Compound X |
| Electron Transport Layer/ Electron Injection Layer | 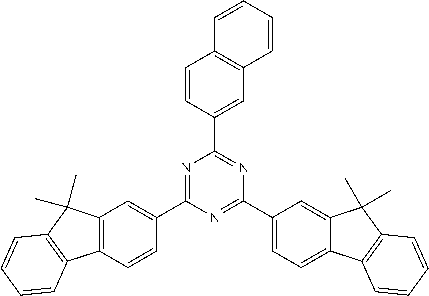 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | ETL-1 |
| | 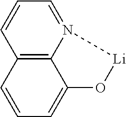 |
| | EIL-1 |

1. An organic electroluminescent compound represented by the following formula 1: 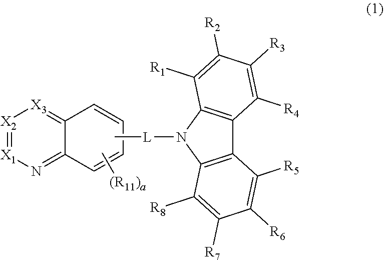
wherein
X₁ to X₃ each independently represent N or CR;
R and R₁₁ each independently represent hydrogen, deuterium, a halogen, a cyano, a substituted or unsubstituted (C1-C30)alkyl, a substituted or unsubstituted (C6-C30)aryl, or a substituted or unsubstituted (3- to 30-membered)heteroaryl;
L represents a single bond, a substituted or unsubstituted (C6-C30)arylene, or a substituted or unsubstituted (3- to 30-membered)heteroarylene; 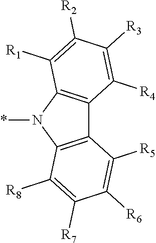
in formula 1 is represented by any one of the following formulas: 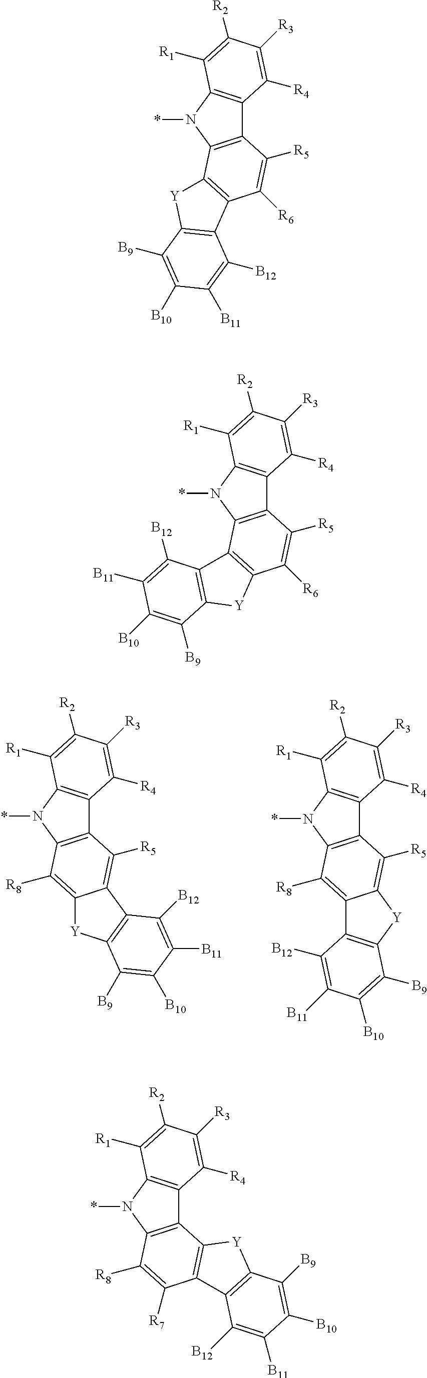 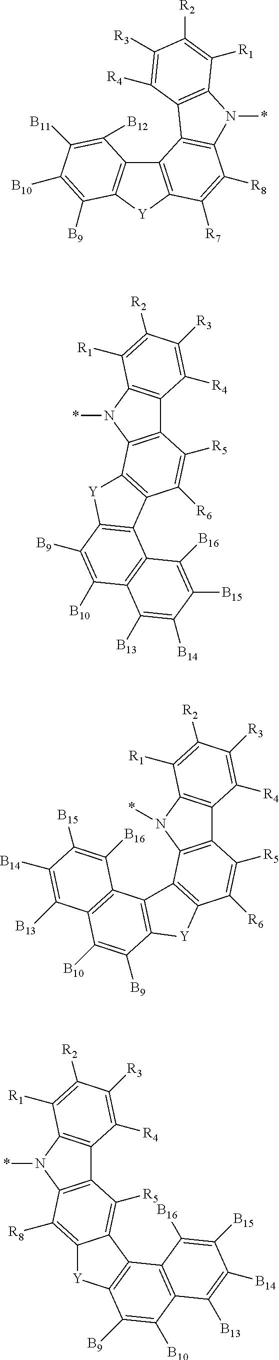 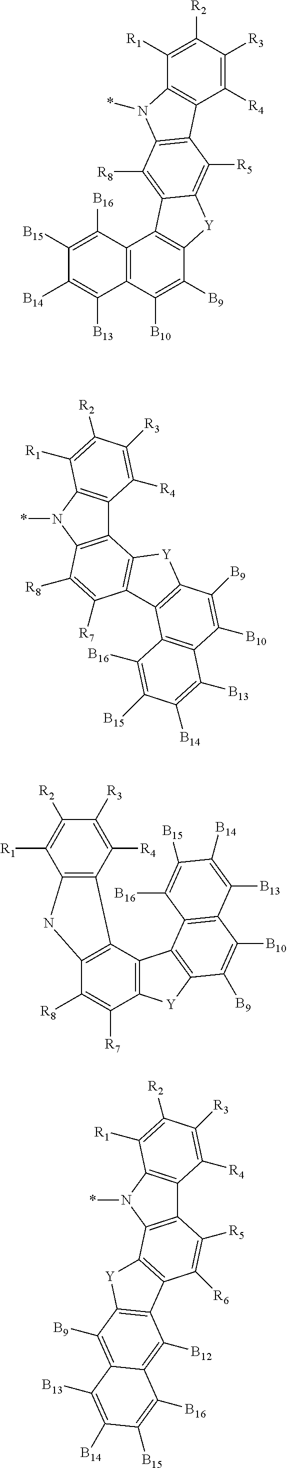 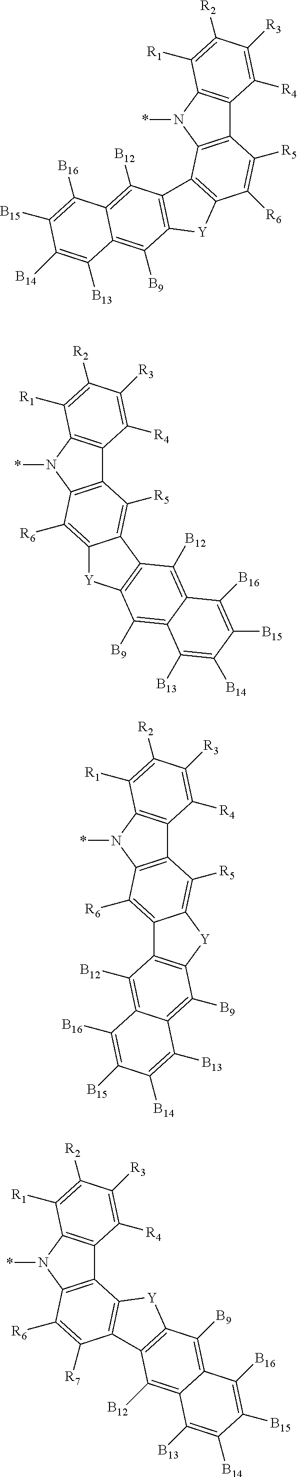 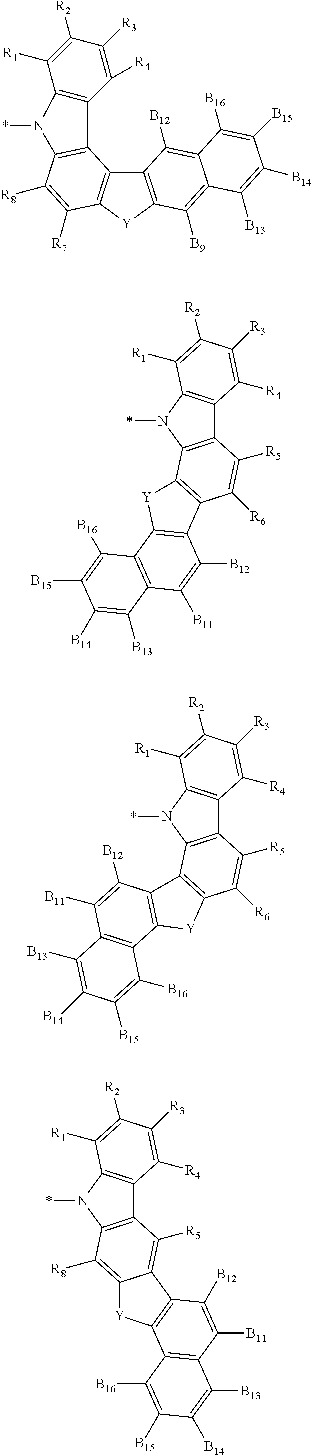 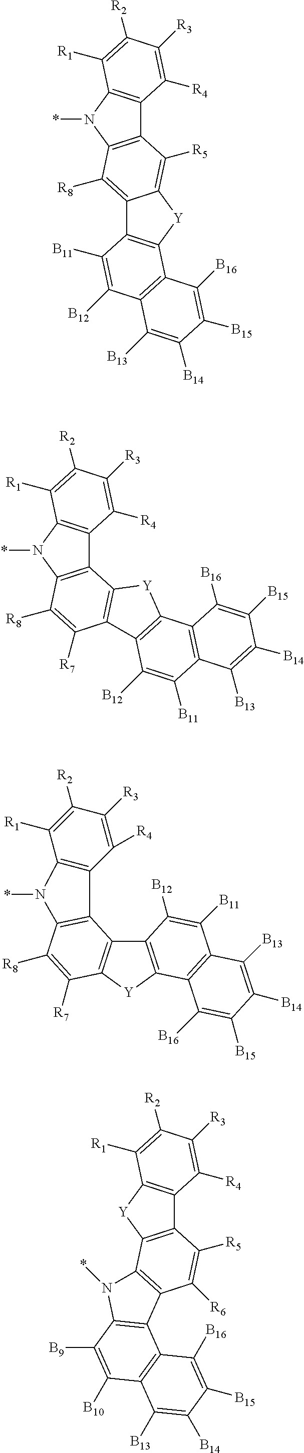 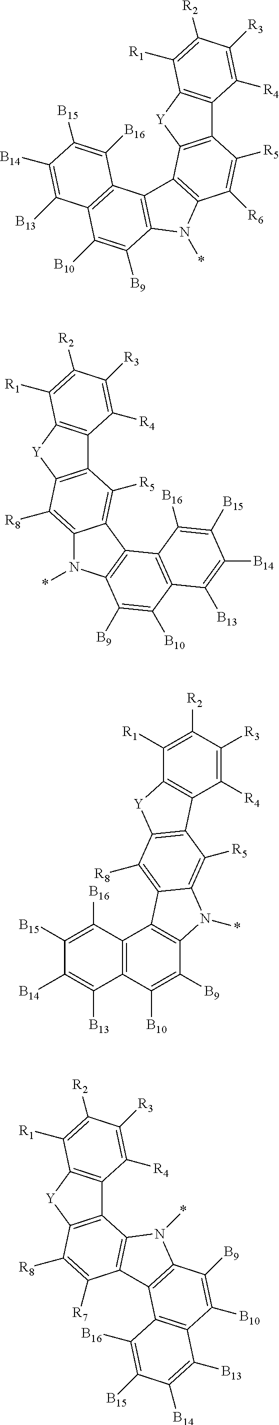 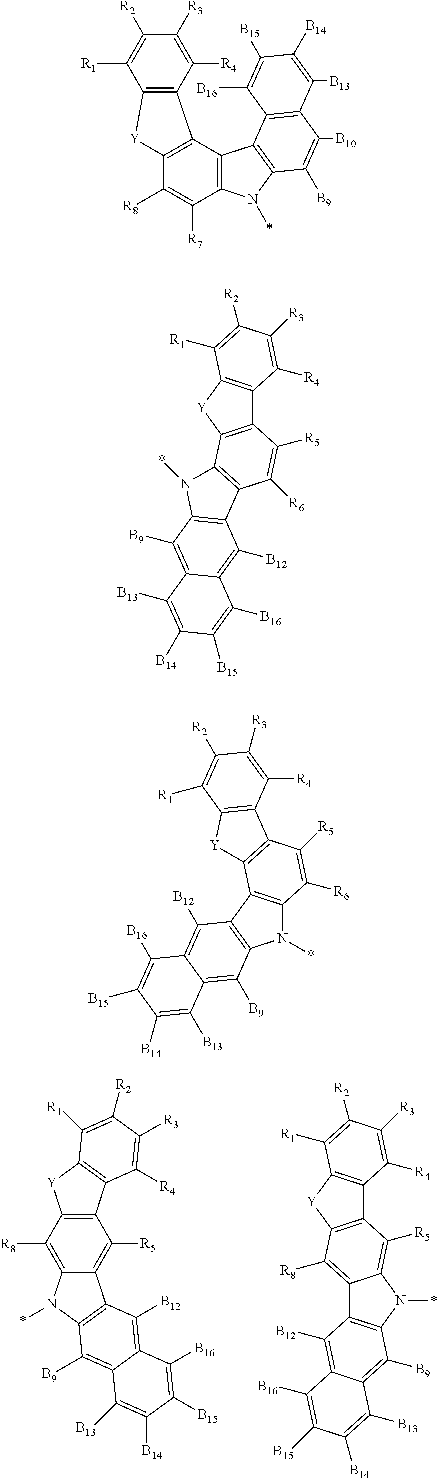 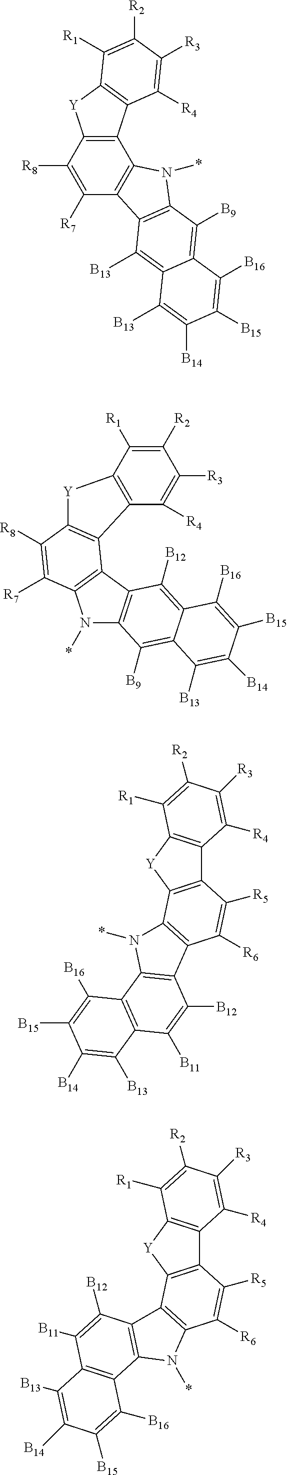 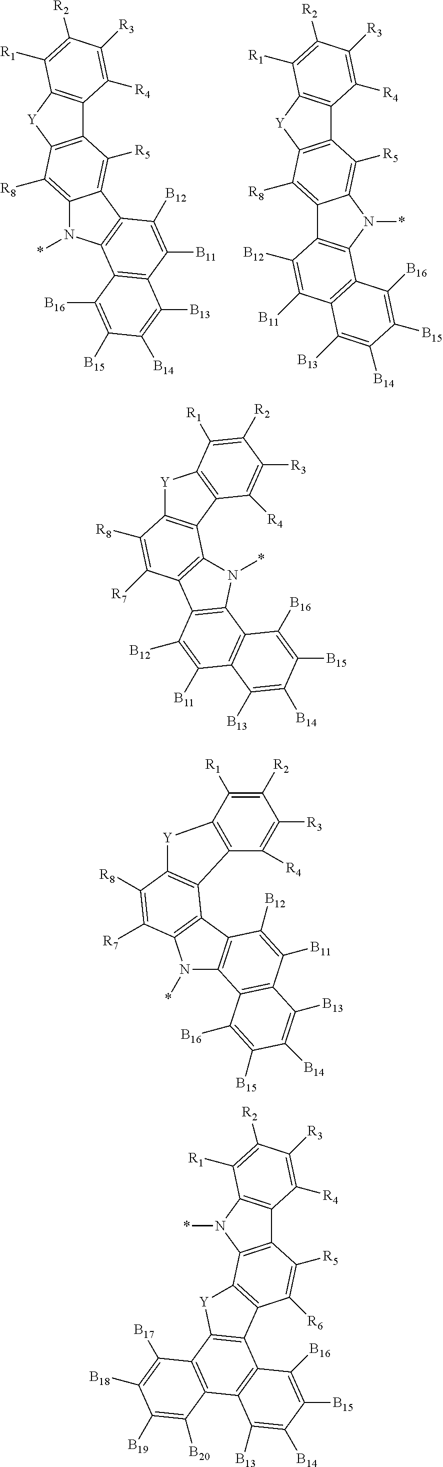 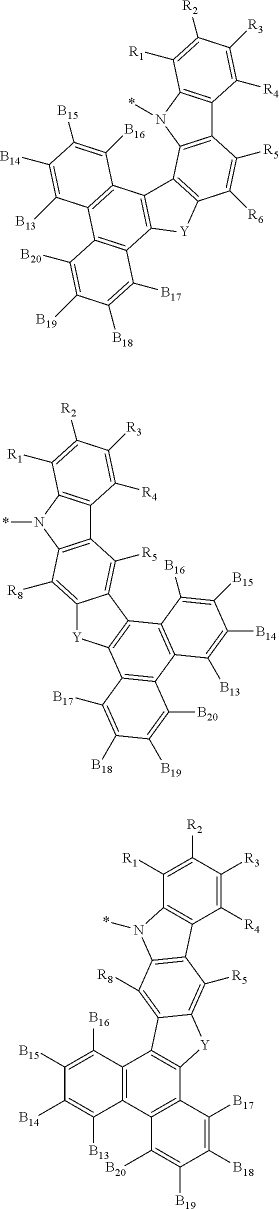 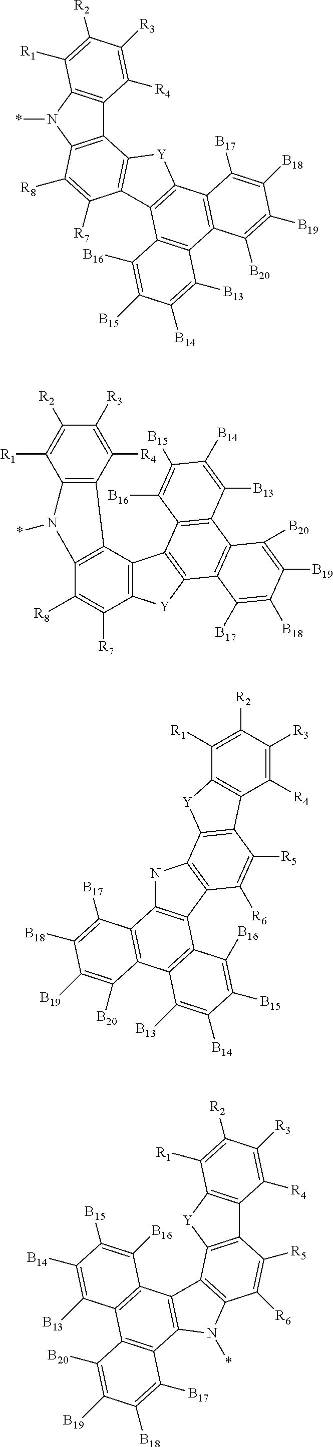 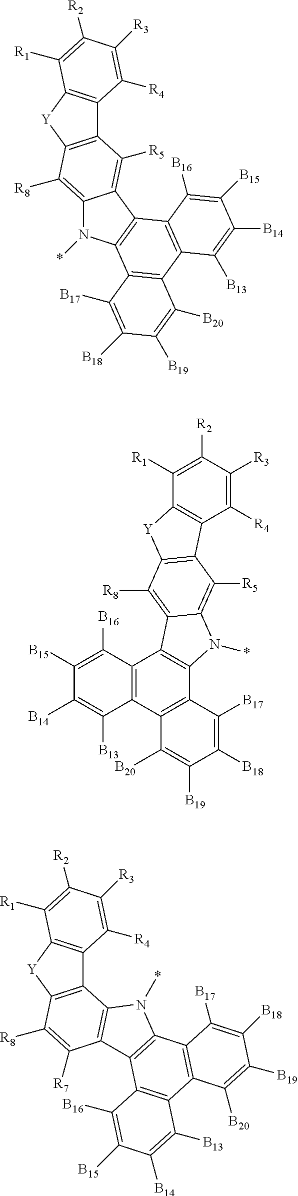 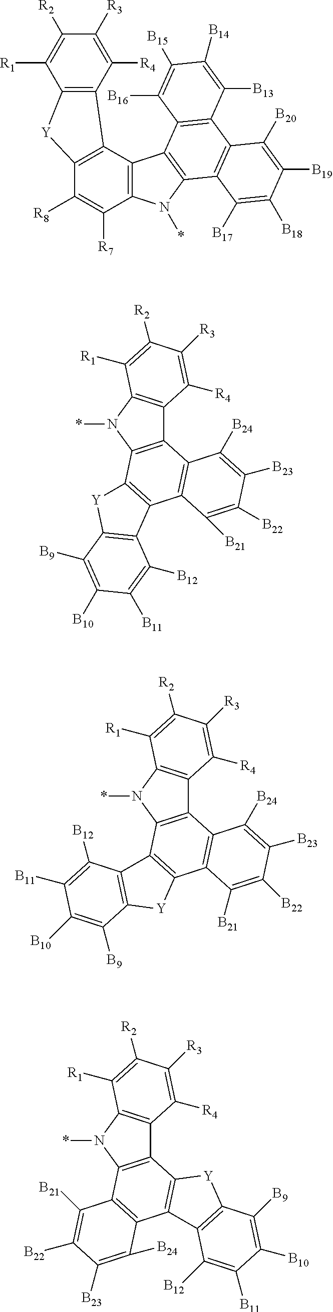 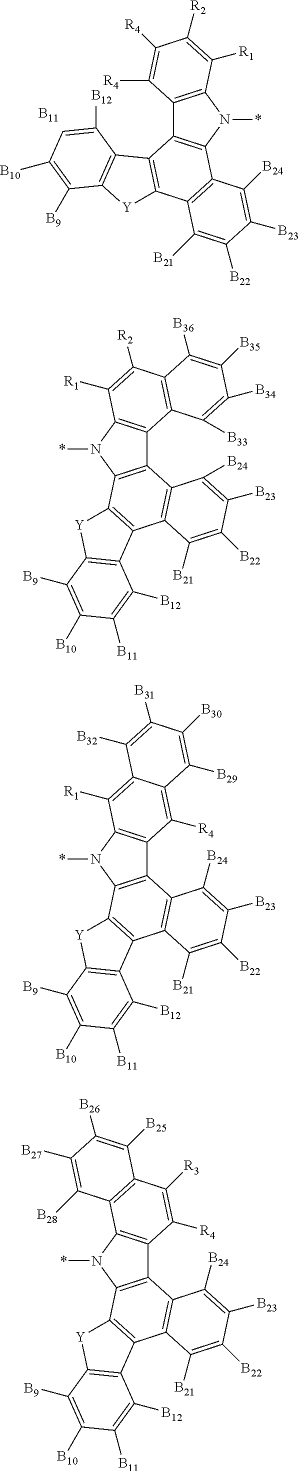 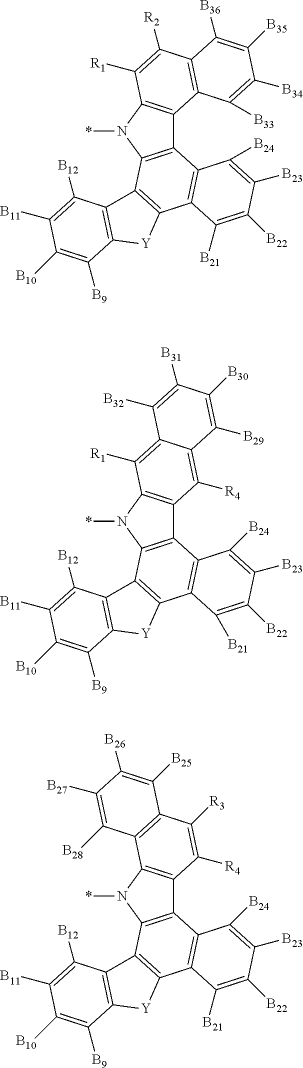 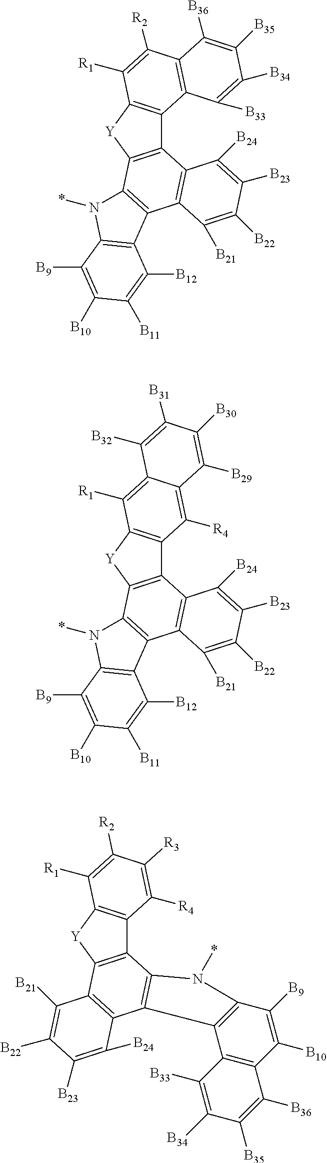 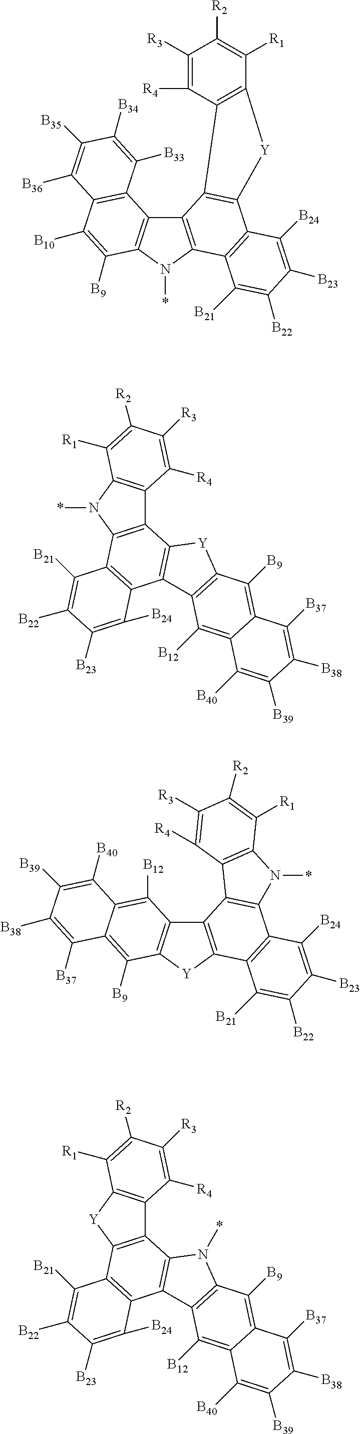 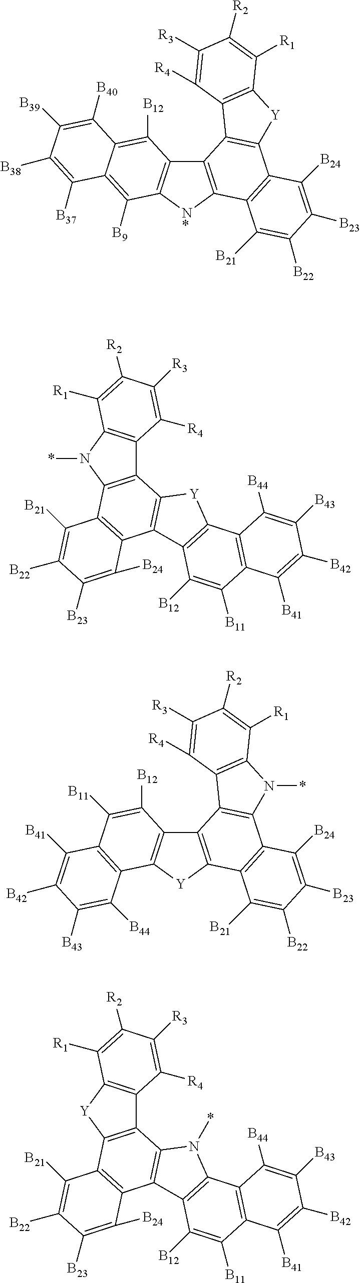 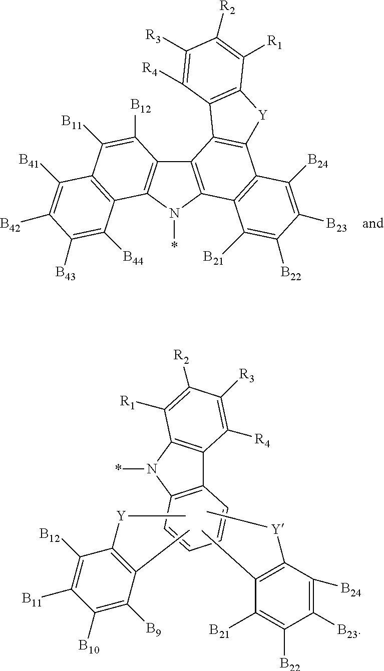
wherein
Y represents N—Ar₂;
Y′ represents N—Ar₂, O, S, or CRₐR_{b};
Ar₂, Rₐ, R_{b}, and B₉ to B₄₄ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, or a substituted or unsubstituted (C1-C30)alkyl(C6-C30)arylamino;
R₁ to R₈ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, or a substituted or unsubstituted (C1-C30)alkyl(C6-C30)arylamino; or may be linked to an adjacent substituent to form a ring; and
a represents an integer of 1 to 3, where a is an integer of 2 or more, each R₁₁ may be the same or different.

2. The organic electroluminescent compound according to claim 1, wherein 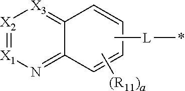
is represented by any one of the following formulas: 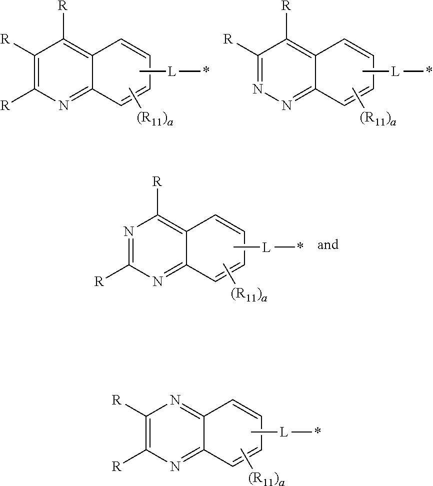
wherein
R, L, R₁₁, and a are as defined in claim 1.

3. The organic electroluminescent compound according to claim 1, wherein the substituents of the substituted (C1-C30)alkyl, the substituted (C6-C30)aryl(ene), the substituted (3- to 30-membered)heteroaryl(ene), the substituted (C3-C30)cycloalkyl, the substituted (C1-C30)alkoxy, the substituted tri(C1-C30)alkylsilyl, the substituted di(C1-C30)alkyl(C6-C30)arylsilyl, the substituted (C1-C30)alkyldi(C6-C30)arylsilyl, the substituted tri(C6-C30)arylsilyl, the substituted mono- or di- (C1-C30)alkylamino, the substituted mono- or di- (C6-C30)arylamino, and the substituted (C1-C30)alkyl(C6-C30)arylamino in R, R₁ to R₈, R₁₁, L, Ar₂, Rₐ, R_{b}, and B, to B₄₄ each independently are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (5- to 30-membered)heteroaryl unsubstituted or substituted with a (C6-C30)aryl(s); a (C6-C30)aryl unsubstituted or substituted with a (5- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di- (C1-C30)alkylamino; a mono- or di- (C6-C30)arylamino unsubstituted or substituted with a (C1-C30)alkyl(s); a (C1-C30)alkyl(C6-C30)arylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

4. The organic electroluminescent compound according to claim 1, wherein
X₁ to X₃ each independently represent N or CR;
R represents hydrogen, or a substituted or unsubstituted (C6-C20)aryl;
R₁₁ represents hydrogen, or a substituted or unsubstituted (C6-C15)aryl;
L represents a single bond, a substituted or unsubstituted (C6-C15)arylene, or a substituted or unsubstituted (5- to 15-membered)heteroarylene;
R₁ to R₈ each independently represent hydrogen, a substituted or unsubstituted (C6-C15)aryl, a substituted or unsubstituted (5- to 15-membered)heteroaryl, or a substituted or unsubstituted di(C6-C15)arylamino; or may be linked to an adjacent substituent to form a ring; and
a represents 1 or 2.

5. The organic electroluminescent compound according to claim 1, wherein
X₁ to X₃ each independently represent N or CR;
R represents hydrogen, or a (C6-C20)aryl unsubstituted or substituted with a (C1-C6)alkyl(s);
R₁₁ represents hydrogen, or an unsubstituted (C6-C15)aryl;
L represents a single bond, an unsubstituted (C6-C15)arylene, or an unsubstituted (5- to 15-membered)heteroarylene;
R₁ to R₈ each independently represent hydrogen; an unsubstituted (C6-C15)aryl; a (5- to 15-membered)heteroaryl unsubstituted or substituted with a (C6-C12)aryl(s); or an unsubstituted di(C6-C15)arylamino; or may be linked to an adjacent substituent to form a ring; and
a represents 1 or 2.

6. The organic electroluminescent compound according to claim 1, wherein the compound represented by formula 1 is selected from the group consisting of:

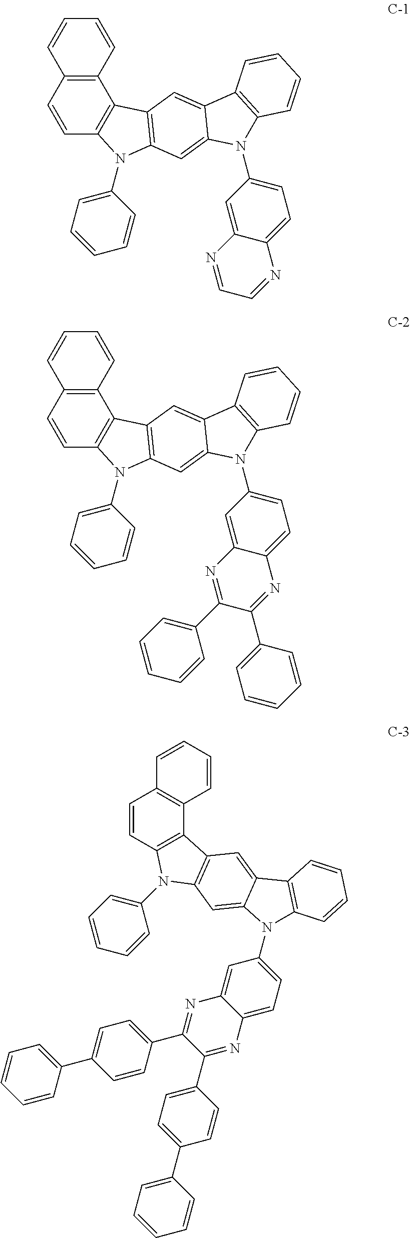 [SMILES: c1ccc(-n2c3cc4c(cc3c3c5ccccc5ccc32)c2ccccc2n4-c2ccc3nccnc3c2)cc1 ; c1ccc(-c2nc3ccc(-n4c5ccccc5c5cc6c7c8ccccc8ccc7n(-c7ccccc7)c6cc54)cc3nc2-c2ccccc2)cc1 ; c1ccc(-c2ccc(-c3nc4ccc(-n5c6ccccc6c6cc7c8c9ccccc9ccc8n(-c8ccccc8)c7cc65)cc4nc3-c3ccc(-c4ccccc4)cc3)cc2)cc1]

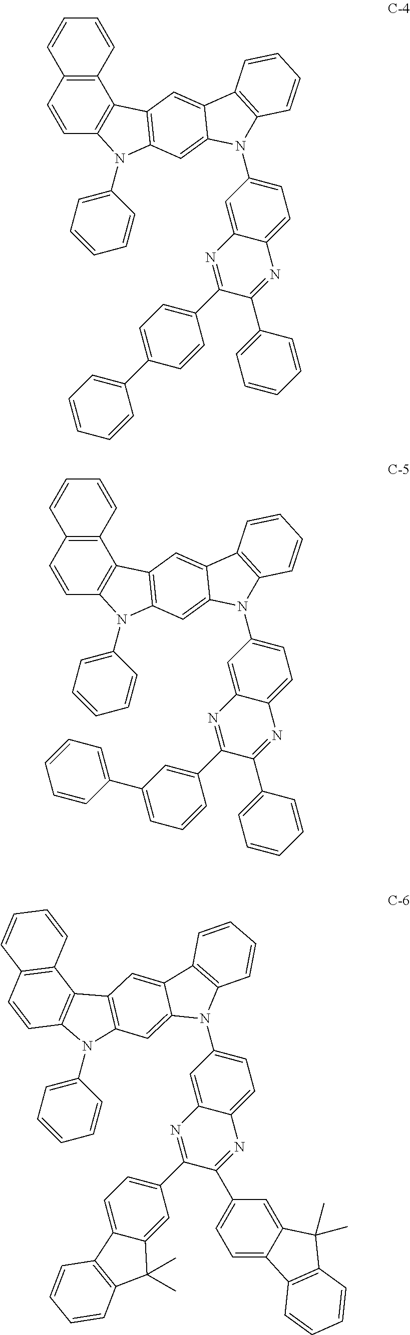

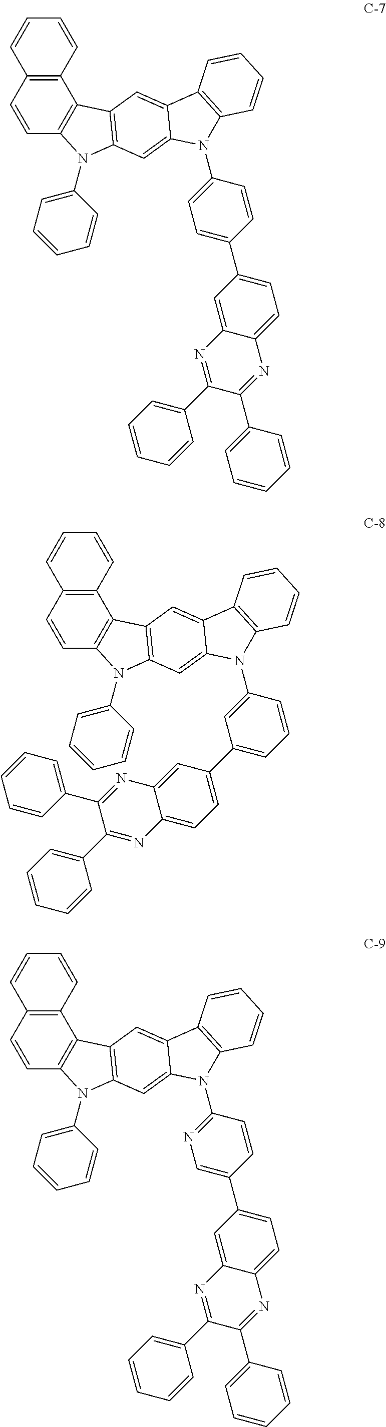

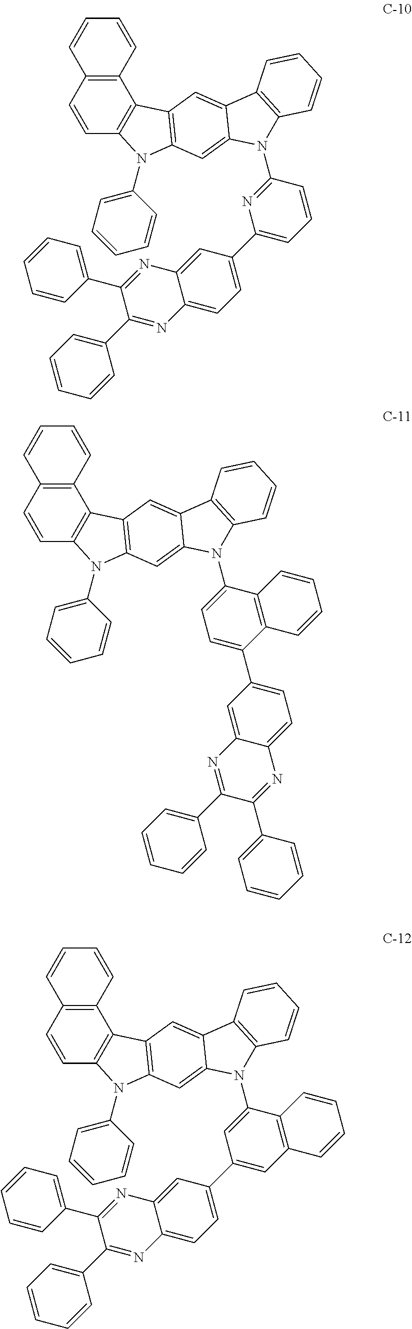

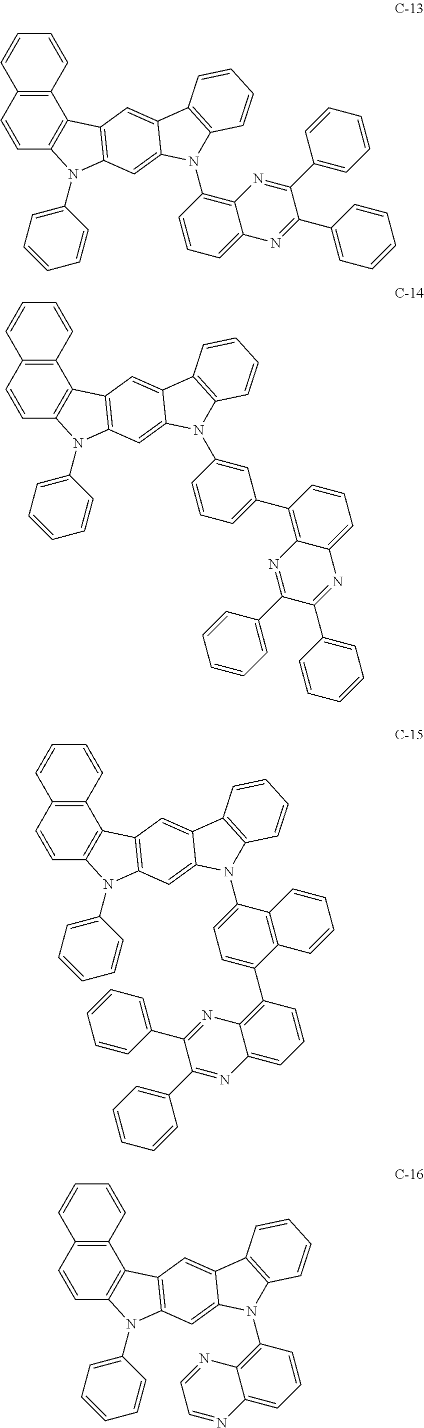

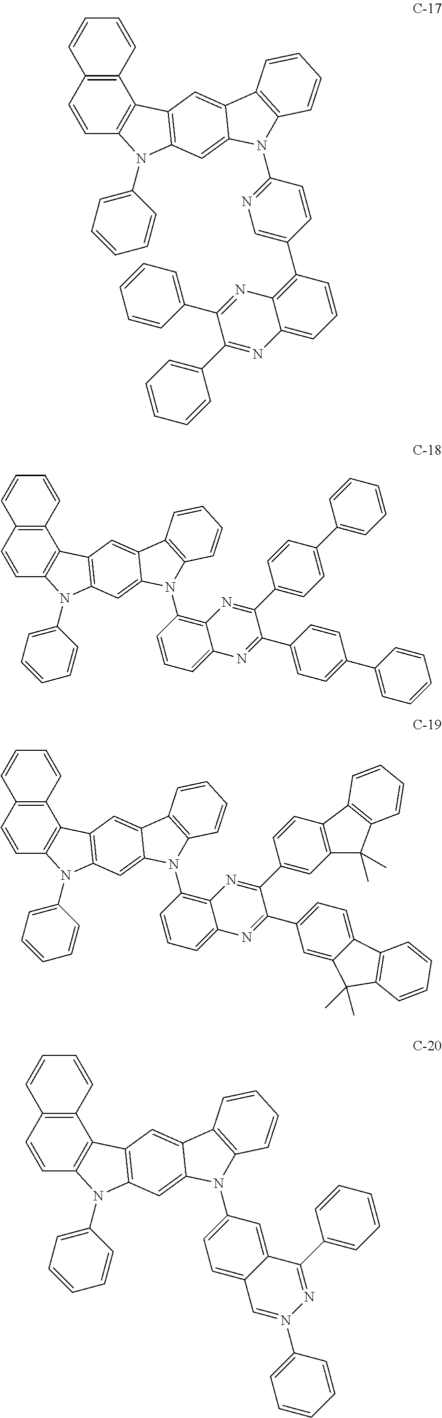

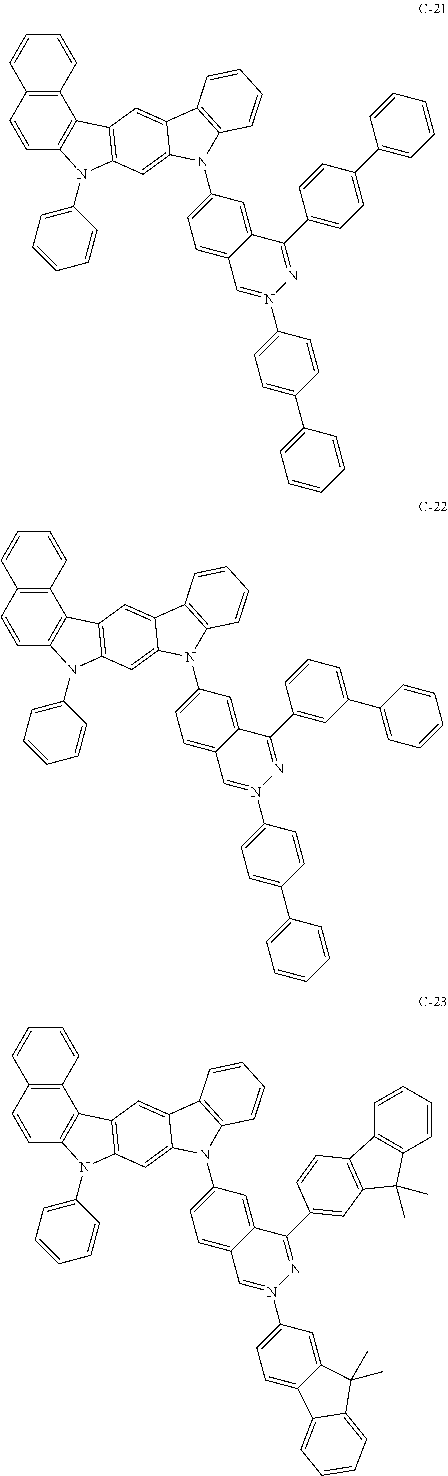

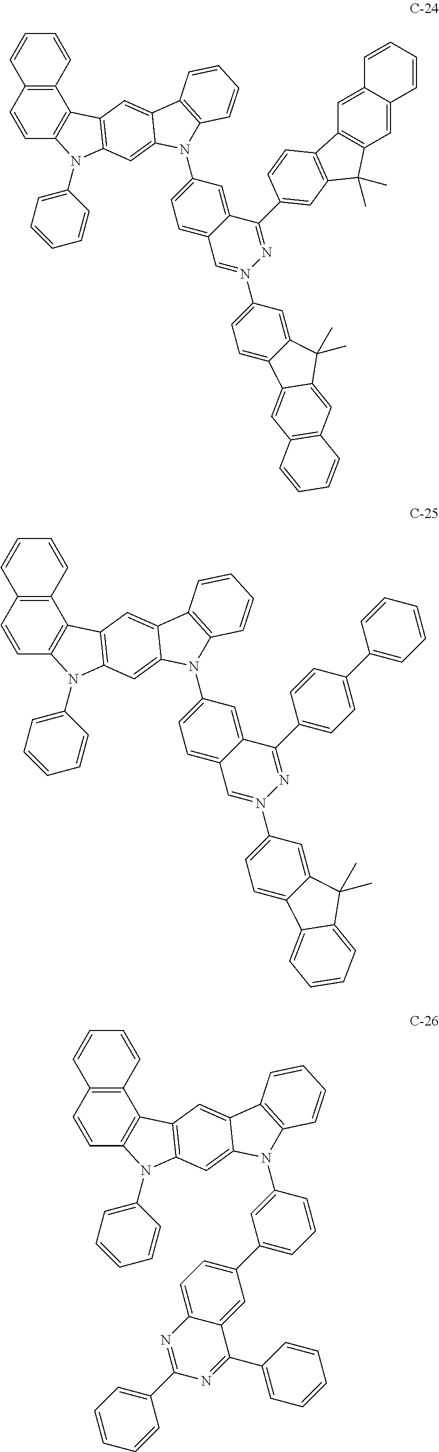

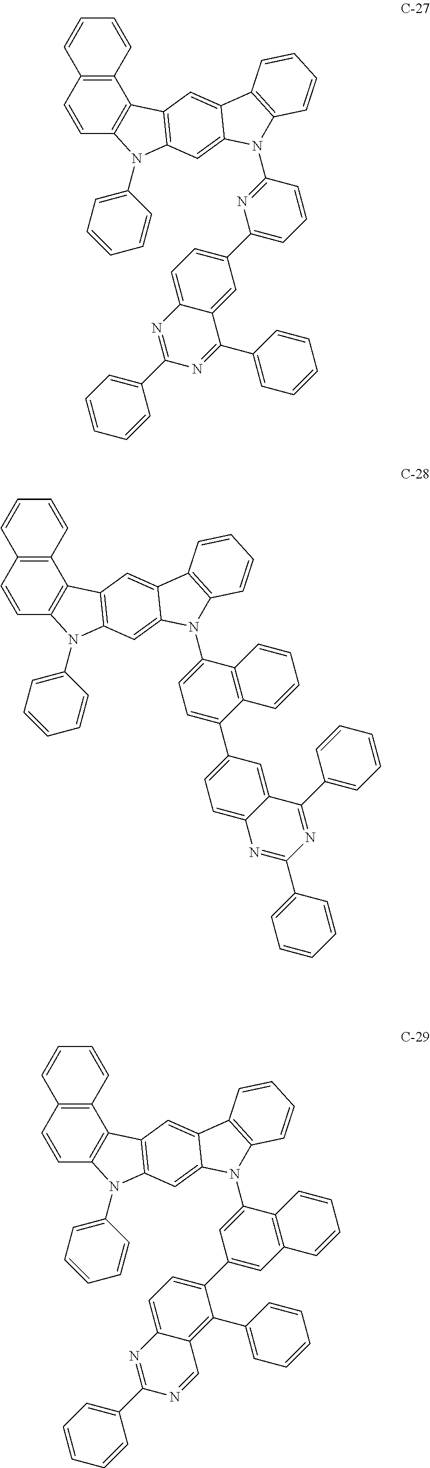 [SMILES: c1ccc(-c2nc(-c3ccccc3)c3cc(-c4cccc(-n5c6ccccc6c6cc7c8c9ccccc9ccc8n(-c8ccccc8)c7cc65)n4)ccc3n2)cc1 ; c1ccc(-c2nc(-c3ccccc3)c3cc(-c4ccc(-n5c6ccccc6c6cc7c8c9ccccc9ccc8n(-c8ccccc8)c7cc65)c5ccccc45)ccc3n2)cc1 ; c1ccc(-c2ncc3c(-c4ccccc4)c(-c4cc(-n5c6ccccc6c6cc7c8c9ccccc9ccc8n(-c8ccccc8)c7cc65)c5ccccc5c4)ccc3n2)cc1]

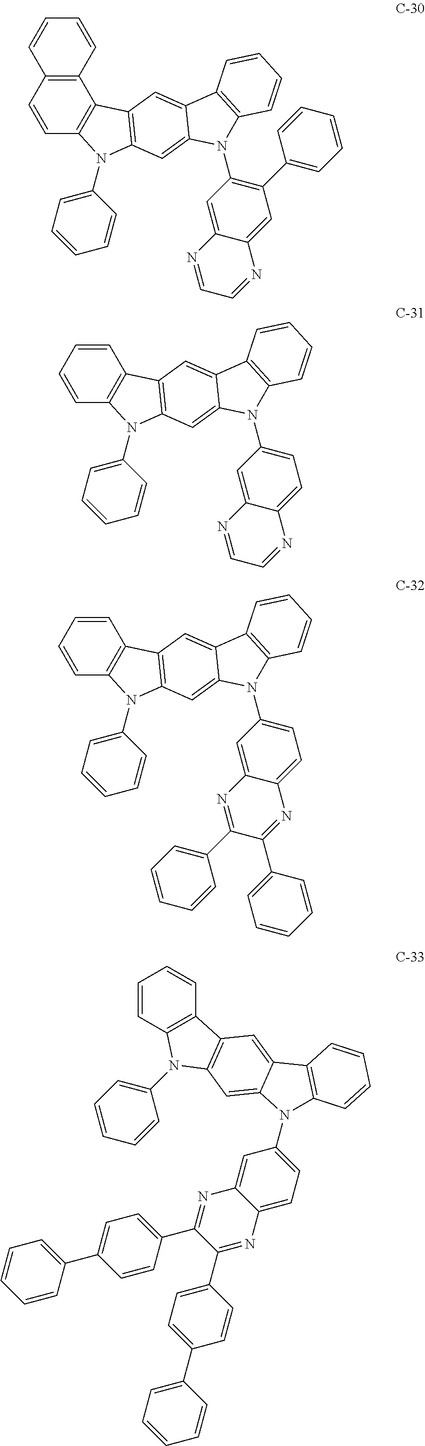

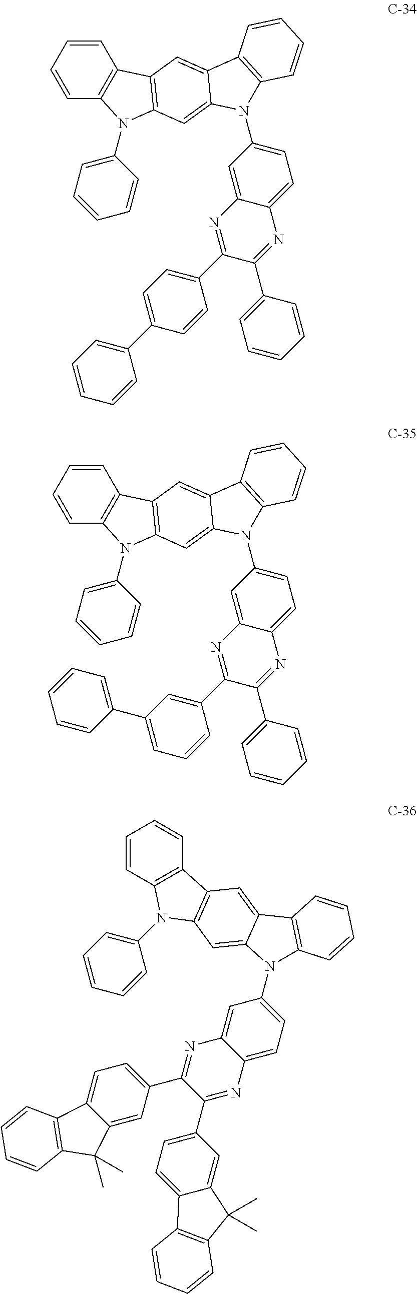 [SMILES: c1ccc(-c2ccc(-c3nc4cc(-n5c6ccccc6c6cc7c8ccccc8n(-c8ccccc8)c7cc65)ccc4nc3-c3ccccc3)cc2)cc1 ; c1ccc(-c2cccc(-c3nc4cc(-n5c6ccccc6c6cc7c8ccccc8n(-c8ccccc8)c7cc65)ccc4nc3-c3ccccc3)c2)cc1 ; CC1(C)c2ccccc2-c2ccc(-c3nc4ccc(-n5c6ccccc6c6cc7c8ccccc8n(-c8ccccc8)c7cc65)cc4nc3-c3ccc4c(c3)C(C)(C)c3ccccc3-4)cc21]

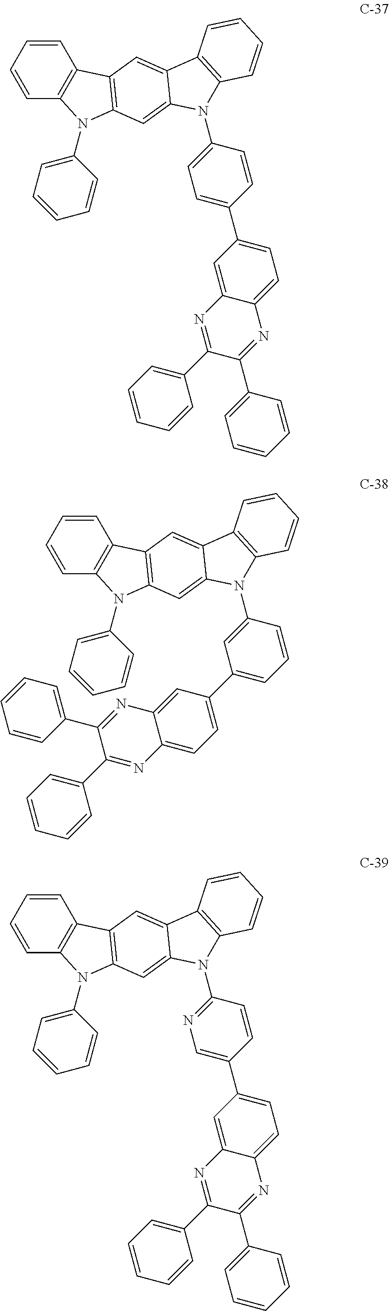

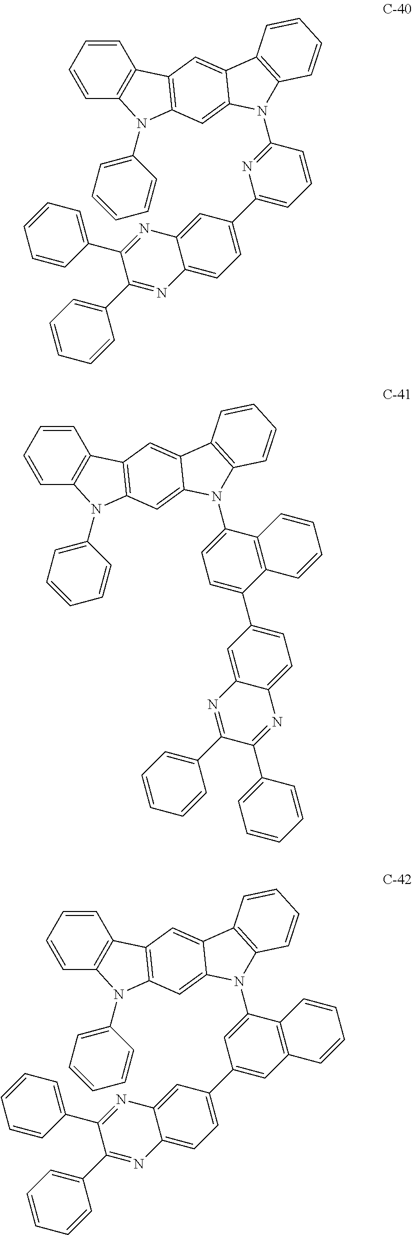 [SMILES: c1ccc(-c2nc3ccc(-c4cccc(-n5c6ccccc6c6cc7c8ccccc8n(-c8ccccc8)c7cc65)n4)cc3nc2-c2ccccc2)cc1 ; c1ccc(-c2nc3ccc(-c4ccc(-n5c6ccccc6c6cc7c8ccccc8n(-c8ccccc8)c7cc65)c5ccccc45)cc3nc2-c2ccccc2)cc1 ; c1ccc(-c2nc3ccc(-c4cc(-n5c6ccccc6c6cc7c8ccccc8n(-c8ccccc8)c7cc65)c5ccccc5c4)cc3nc2-c2ccccc2)cc1]

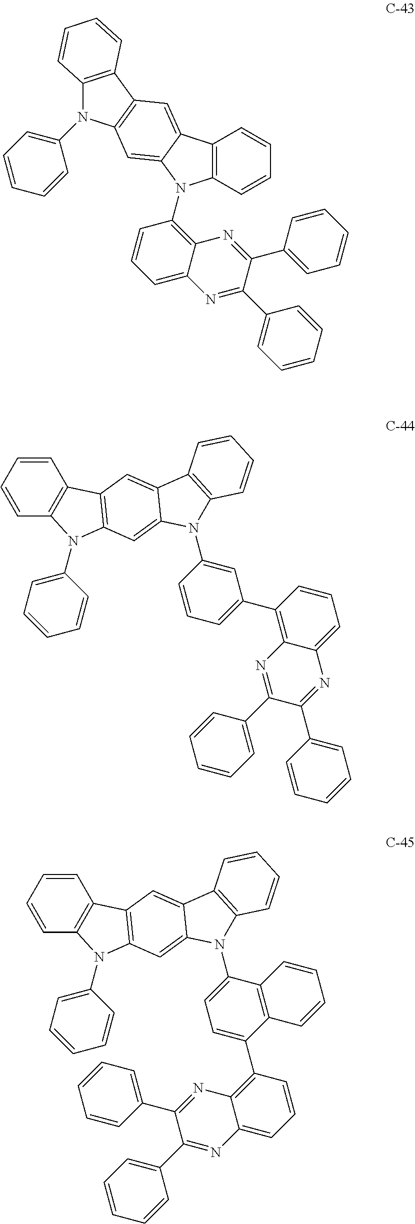 [SMILES: c1ccc(-c2nc3cccc(-n4c5ccccc5c5cc6c7ccccc7n(-c7ccccc7)c6cc54)c3nc2-c2ccccc2)cc1 ; c1ccc(-c2nc3cccc(-c4cccc(-n5c6ccccc6c6cc7c8ccccc8n(-c8ccccc8)c7cc65)c4)c3nc2-c2ccccc2)cc1 ; c1ccc(-c2nc3cccc(-c4ccc(-n5c6ccccc6c6cc7c8ccccc8n(-c8ccccc8)c7cc65)c5ccccc45)c3nc2-c2ccccc2)cc1]

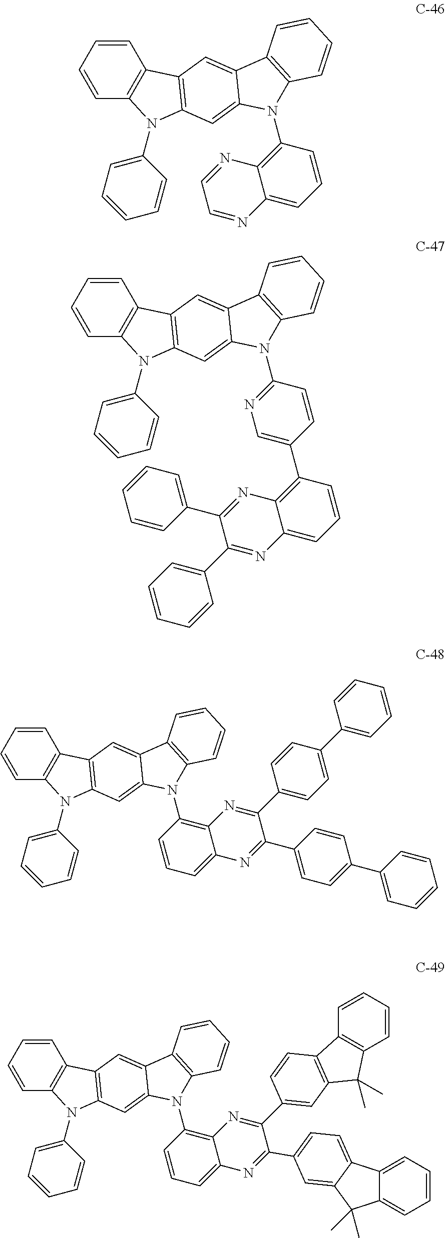 [SMILES: c1ccc(-n2c3ccccc3c3cc4c5ccccc5n(-c5cccc6nccnc56)c4cc32)cc1 ; c1ccc(-c2nc3cccc(-c4ccc(-n5c6ccccc6c6cc7c8ccccc8n(-c8ccccc8)c7cc65)nc4)c3nc2-c2ccccc2)cc1 ; c1ccc(-c2ccc(-c3nc4cccc(-n5c6ccccc6c6cc7c8ccccc8n(-c8ccccc8)c7cc65)c4nc3-c3ccc(-c4ccccc4)cc3)cc2)cc1 ; CC1(C)c2ccccc2-c2ccc(-c3nc4cccc(-n5c6ccccc6c6cc7c8ccccc8n(-c8ccccc8)c7cc65)c4nc3-c3ccc4c(c3)C(C)(C)c3ccccc3-4)cc21]

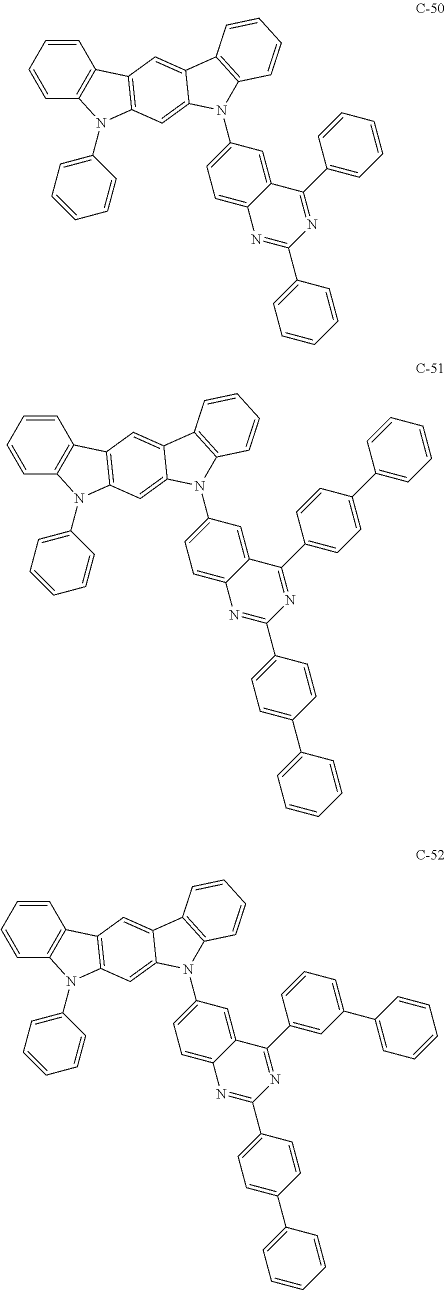 [SMILES: c1ccc(-c2nc(-c3ccccc3)c3cc(-n4c5ccccc5c5cc6c7ccccc7n(-c7ccccc7)c6cc54)ccc3n2)cc1 ; c1ccc(-c2ccc(-c3nc(-c4ccc(-c5ccccc5)cc4)c4cc(-n5c6ccccc6c6cc7c8ccccc8n(-c8ccccc8)c7cc65)ccc4n3)cc2)cc1 ; c1ccc(-c2ccc(-c3nc(-c4cccc(-c5ccccc5)c4)c4cc(-n5c6ccccc6c6cc7c8ccccc8n(-c8ccccc8)c7cc65)ccc4n3)cc2)cc1]

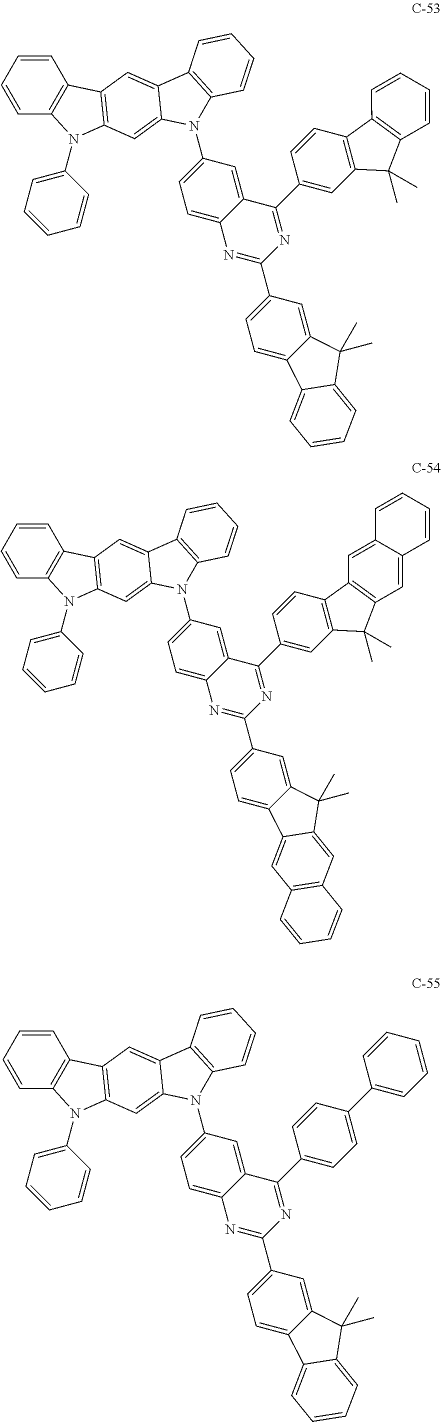 [SMILES: CC1(C)c2ccccc2-c2ccc(-c3nc(-c4ccc5c(c4)C(C)(C)c4ccccc4-5)c4cc(-n5c6ccccc6c6cc7c8ccccc8n(-c8ccccc8)c7cc65)ccc4n3)cc21 ; CC1(C)c2cc(-c3nc(-c4ccc5c(c4)C(C)(C)c4cc6ccccc6cc4-5)c4cc(-n5c6ccccc6c6cc7c8ccccc8n(-c8ccccc8)c7cc65)ccc4n3)ccc2-c2cc3ccccc3cc21 ; CC1(C)c2ccccc2-c2ccc(-c3nc(-c4ccc(-c5ccccc5)cc4)c4cc(-n5c6ccccc6c6cc7c8ccccc8n(-c8ccccc8)c7cc65)ccc4n3)cc21]

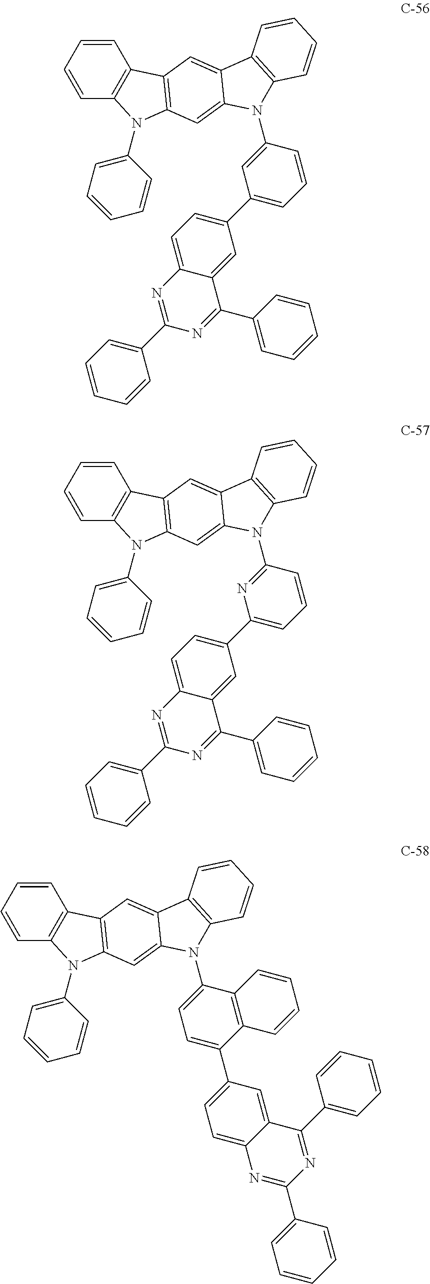 [SMILES: c1ccc(-c2nc(-c3ccccc3)c3cc(-c4cccc(-n5c6ccccc6c6cc7c8ccccc8n(-c8ccccc8)c7cc65)c4)ccc3n2)cc1 ; c1ccc(-c2nc(-c3ccccc3)c3cc(-c4cccc(-n5c6ccccc6c6cc7c8ccccc8n(-c8ccccc8)c7cc65)n4)ccc3n2)cc1 ; c1ccc(-c2nc(-c3ccccc3)c3cc(-c4ccc(-n5c6ccccc6c6cc7c8ccccc8n(-c8ccccc8)c7cc65)c5ccccc45)ccc3n2)cc1]

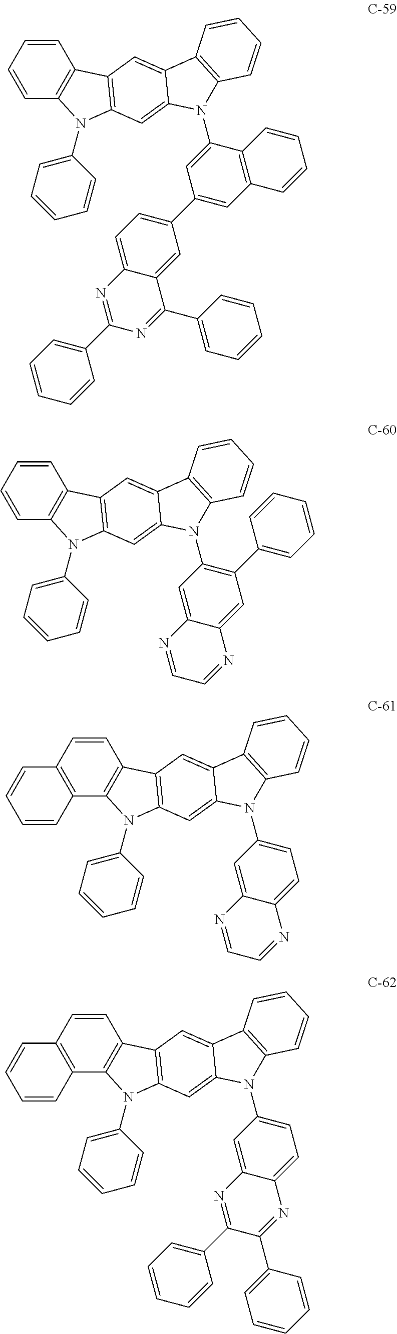

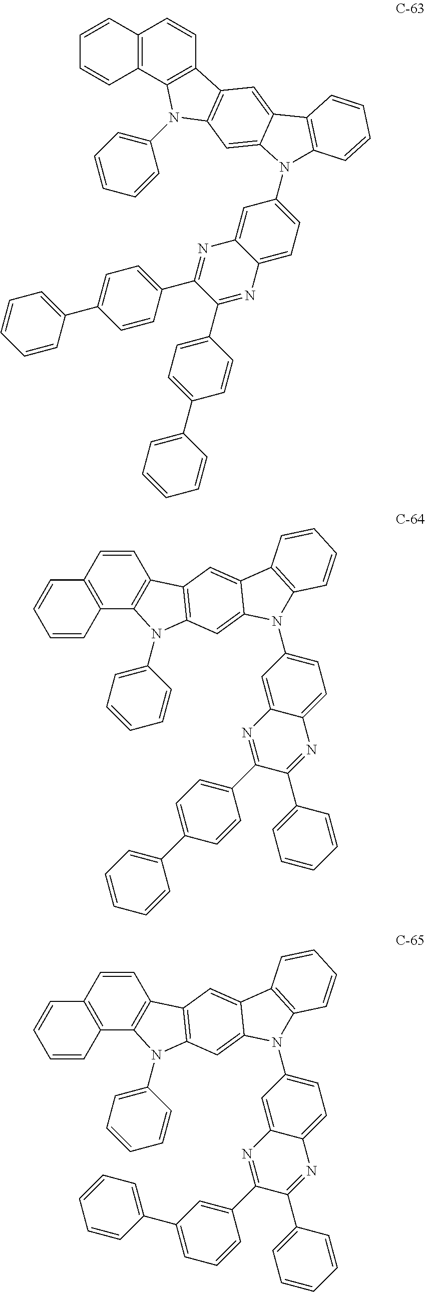 [SMILES: c1ccc(-c2ccc(-c3nc4ccc(-n5c6ccccc6c6cc7c8ccc9ccccc9c8n(-c8ccccc8)c7cc65)cc4nc3-c3ccc(-c4ccccc4)cc3)cc2)cc1 ; c1ccc(-c2ccc(-c3nc4cc(-n5c6ccccc6c6cc7c8ccc9ccccc9c8n(-c8ccccc8)c7cc65)ccc4nc3-c3ccccc3)cc2)cc1 ; c1ccc(-c2cccc(-c3nc4cc(-n5c6ccccc6c6cc7c8ccc9ccccc9c8n(-c8ccccc8)c7cc65)ccc4nc3-c3ccccc3)c2)cc1]

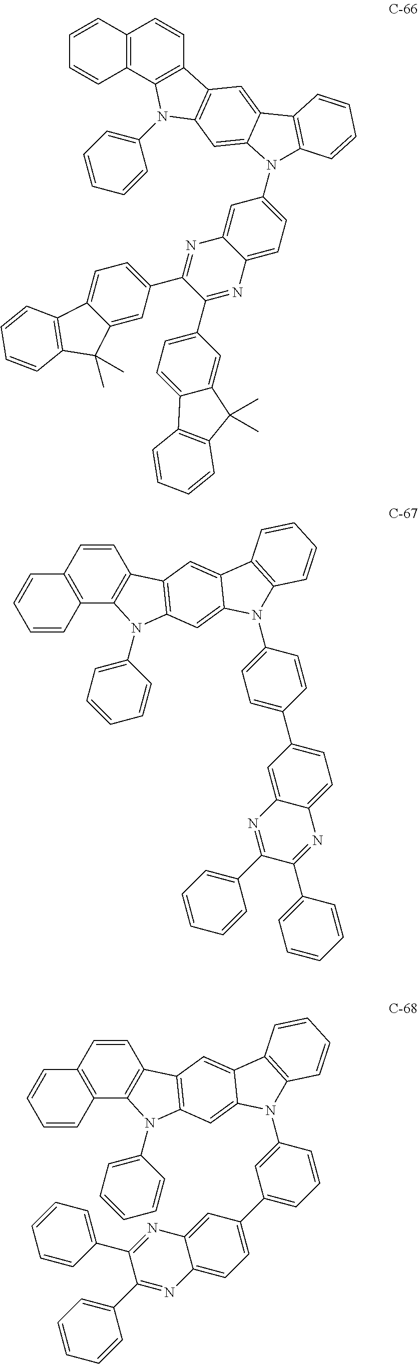 [SMILES: CC1(C)c2ccccc2-c2ccc(-c3nc4ccc(-n5c6ccccc6c6cc7c8ccc9ccccc9c8n(-c8ccccc8)c7cc65)cc4nc3-c3ccc4c(c3)C(C)(C)c3ccccc3-4)cc21 ; c1ccc(-c2nc3ccc(-c4ccc(-n5c6ccccc6c6cc7c8ccc9ccccc9c8n(-c8ccccc8)c7cc65)cc4)cc3nc2-c2ccccc2)cc1 ; c1ccc(-c2nc3ccc(-c4cccc(-n5c6ccccc6c6cc7c8ccc9ccccc9c8n(-c8ccccc8)c7cc65)c4)cc3nc2-c2ccccc2)cc1]

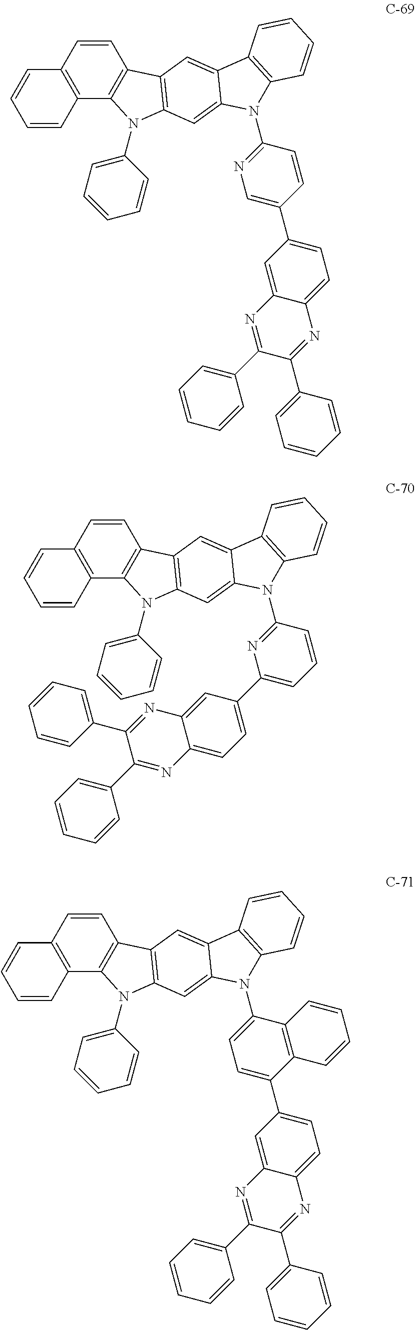

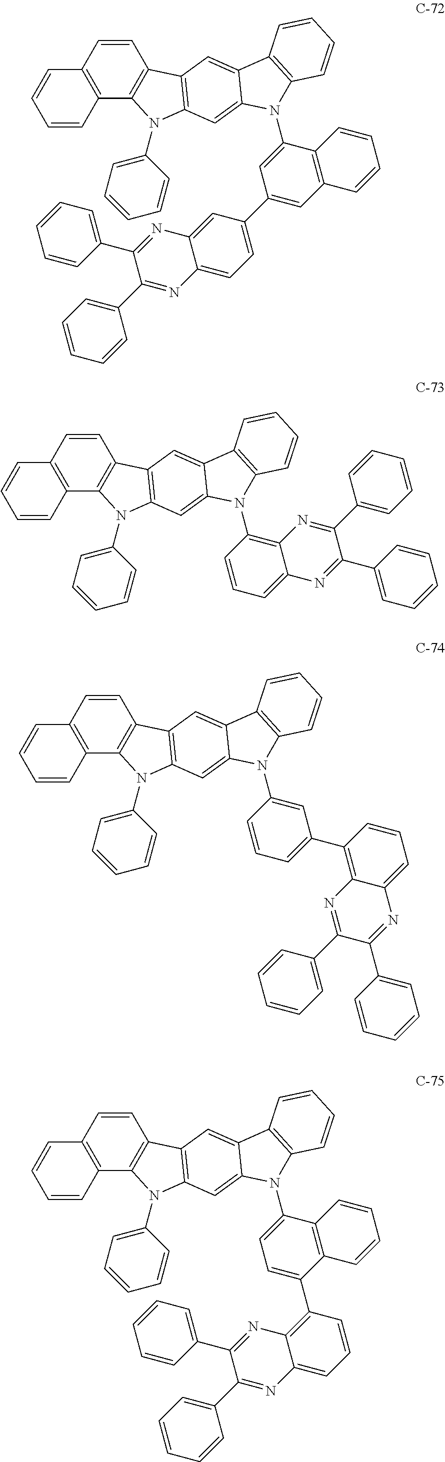 [SMILES: c1ccc(-c2nc3ccc(-c4cc(-n5c6ccccc6c6cc7c8ccc9ccccc9c8n(-c8ccccc8)c7cc65)c5ccccc5c4)cc3nc2-c2ccccc2)cc1 ; c1ccc(-c2nc3cccc(-n4c5ccccc5c5cc6c7ccc8ccccc8c7n(-c7ccccc7)c6cc54)c3nc2-c2ccccc2)cc1 ; c1ccc(-c2nc3cccc(-c4cccc(-n5c6ccccc6c6cc7c8ccc9ccccc9c8n(-c8ccccc8)c7cc65)c4)c3nc2-c2ccccc2)cc1 ; c1ccc(-c2nc3cccc(-c4ccc(-n5c6ccccc6c6cc7c8ccc9ccccc9c8n(-c8ccccc8)c7cc65)c5ccccc45)c3nc2-c2ccccc2)cc1]

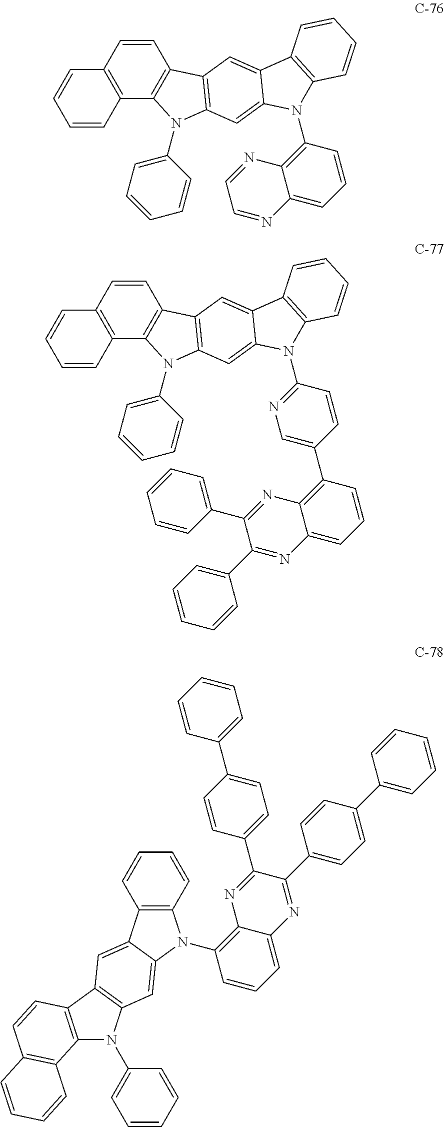 [SMILES: c1ccc(-n2c3cc4c(cc3c3ccc5ccccc5c32)c2ccccc2n4-c2cccc3nccnc23)cc1 ; c1ccc(-c2nc3cccc(-c4ccc(-n5c6ccccc6c6cc7c8ccc9ccccc9c8n(-c8ccccc8)c7cc65)nc4)c3nc2-c2ccccc2)cc1 ; c1ccc(-c2ccc(-c3nc4cccc(-n5c6ccccc6c6cc7c8ccc9ccccc9c8n(-c8ccccc8)c7cc65)c4nc3-c3ccc(-c4ccccc4)cc3)cc2)cc1]

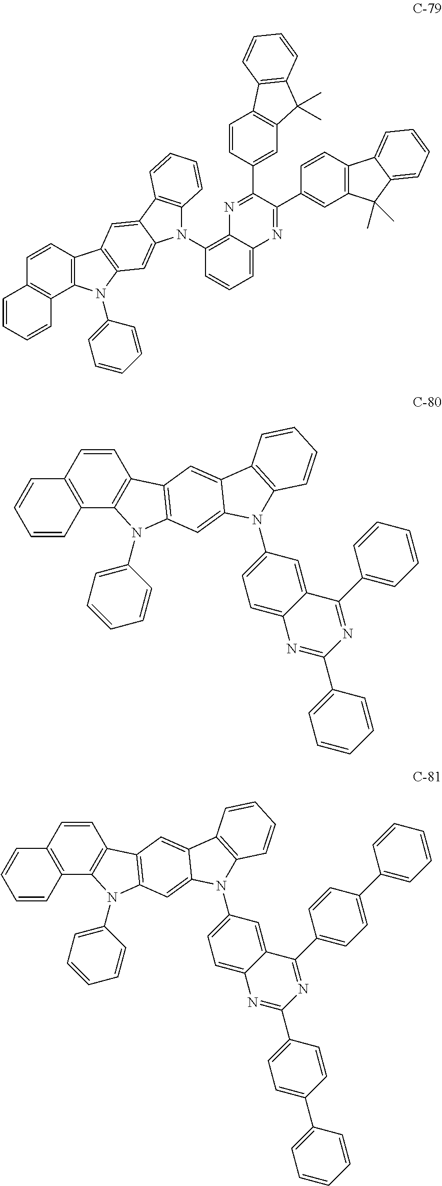 [SMILES: CC1(C)c2ccccc2-c2ccc(-c3nc4cccc(-n5c6ccccc6c6cc7c8ccc9ccccc9c8n(-c8ccccc8)c7cc65)c4nc3-c3ccc4c(c3)C(C)(C)c3ccccc3-4)cc21 ; c1ccc(-c2nc(-c3ccccc3)c3cc(-n4c5ccccc5c5cc6c7ccc8ccccc8c7n(-c7ccccc7)c6cc54)ccc3n2)cc1 ; c1ccc(-c2ccc(-c3nc(-c4ccc(-c5ccccc5)cc4)c4cc(-n5c6ccccc6c6cc7c8ccc9ccccc9c8n(-c8ccccc8)c7cc65)ccc4n3)cc2)cc1]

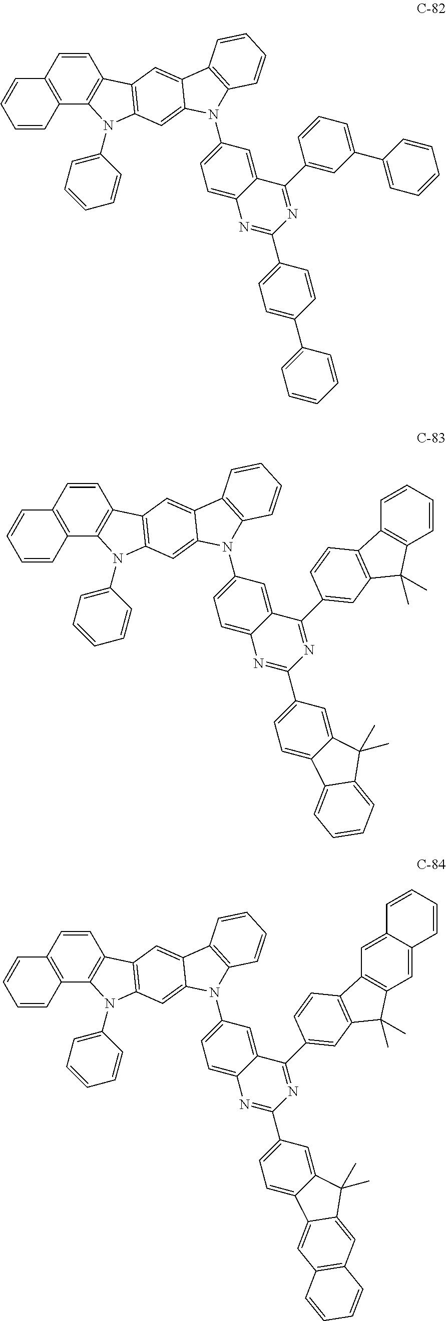 [SMILES: c1ccc(-c2ccc(-c3nc(-c4cccc(-c5ccccc5)c4)c4cc(-n5c6ccccc6c6cc7c8ccc9ccccc9c8n(-c8ccccc8)c7cc65)ccc4n3)cc2)cc1 ; CC1(C)c2ccccc2-c2ccc(-c3nc(-c4ccc5c(c4)C(C)(C)c4ccccc4-5)c4cc(-n5c6ccccc6c6cc7c8ccc9ccccc9c8n(-c8ccccc8)c7cc65)ccc4n3)cc21 ; CC1(C)c2cc(-c3nc(-c4ccc5c(c4)C(C)(C)c4cc6ccccc6cc4-5)c4cc(-n5c6ccccc6c6cc7c8ccc9ccccc9c8n(-c8ccccc8)c7cc65)ccc4n3)ccc2-c2cc3ccccc3cc21]

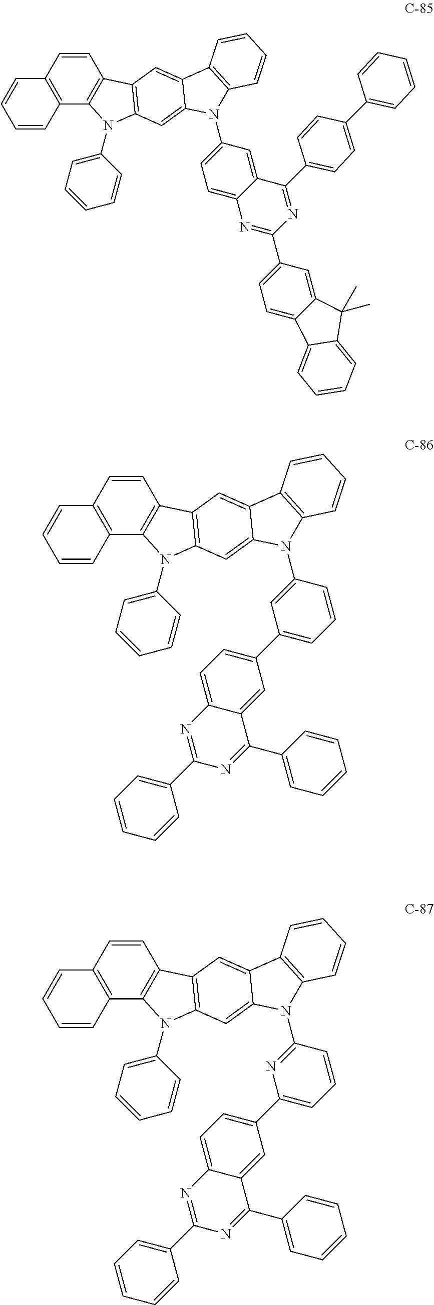 [SMILES: CC1(C)c2ccccc2-c2ccc(-c3nc(-c4ccc(-c5ccccc5)cc4)c4cc(-n5c6ccccc6c6cc7c8ccc9ccccc9c8n(-c8ccccc8)c7cc65)ccc4n3)cc21 ; c1ccc(-c2nc(-c3ccccc3)c3cc(-c4cccc(-n5c6ccccc6c6cc7c8ccc9ccccc9c8n(-c8ccccc8)c7cc65)c4)ccc3n2)cc1 ; c1ccc(-c2nc(-c3ccccc3)c3cc(-c4cccc(-n5c6ccccc6c6cc7c8ccc9ccccc9c8n(-c8ccccc8)c7cc65)n4)ccc3n2)cc1]

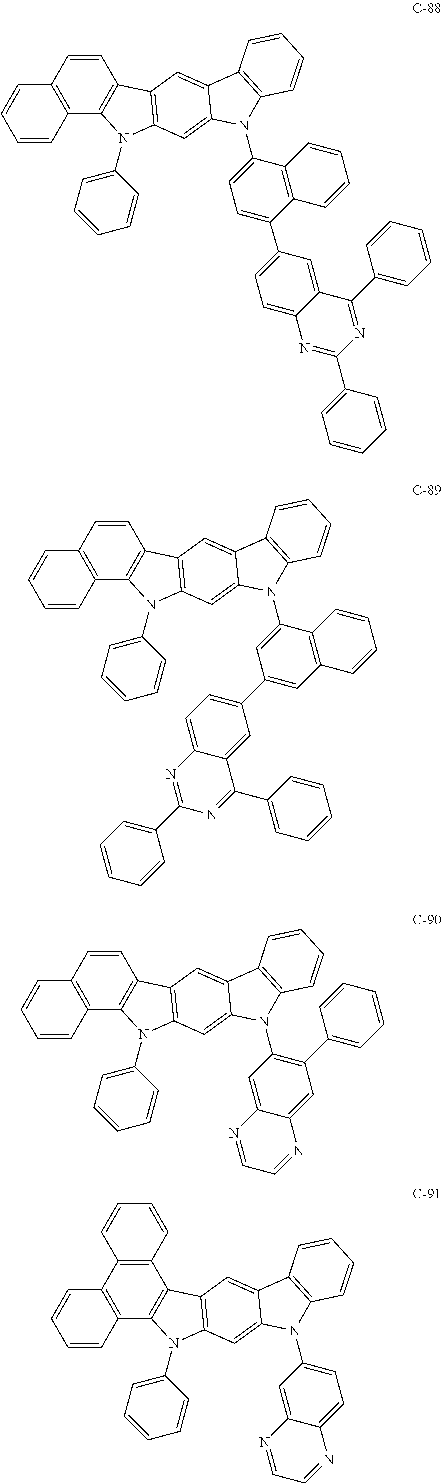 [SMILES: c1ccc(-c2nc(-c3ccccc3)c3cc(-c4ccc(-n5c6ccccc6c6cc7c8ccc9ccccc9c8n(-c8ccccc8)c7cc65)c5ccccc45)ccc3n2)cc1 ; c1ccc(-c2nc(-c3ccccc3)c3cc(-c4cc(-n5c6ccccc6c6cc7c8ccc9ccccc9c8n(-c8ccccc8)c7cc65)c5ccccc5c4)ccc3n2)cc1 ; c1ccc(-c2cc3nccnc3cc2-n2c3ccccc3c3cc4c5ccc6ccccc6c5n(-c5ccccc5)c4cc32)cc1 ; c1ccc(-n2c3cc4c(cc3c3c5ccccc5c5ccccc5c32)c2ccccc2n4-c2ccc3nccnc3c2)cc1]

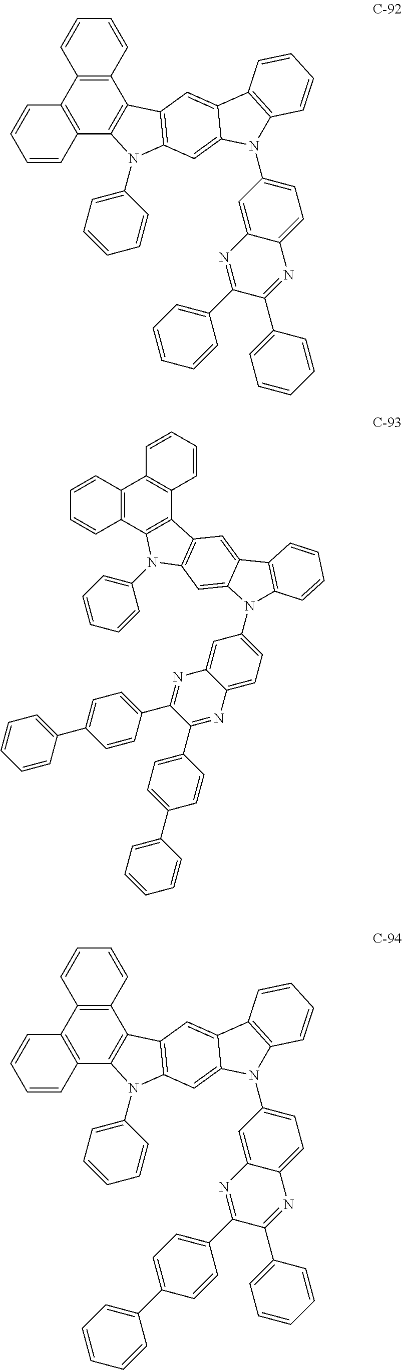 [SMILES: c1ccc(-c2nc3ccc(-n4c5ccccc5c5cc6c7c8ccccc8c8ccccc8c7n(-c7ccccc7)c6cc54)cc3nc2-c2ccccc2)cc1 ; c1ccc(-c2ccc(-c3nc4ccc(-n5c6ccccc6c6cc7c8c9ccccc9c9ccccc9c8n(-c8ccccc8)c7cc65)cc4nc3-c3ccc(-c4ccccc4)cc3)cc2)cc1 ; c1ccc(-c2ccc(-c3nc4cc(-n5c6ccccc6c6cc7c8c9ccccc9c9ccccc9c8n(-c8ccccc8)c7cc65)ccc4nc3-c3ccccc3)cc2)cc1]

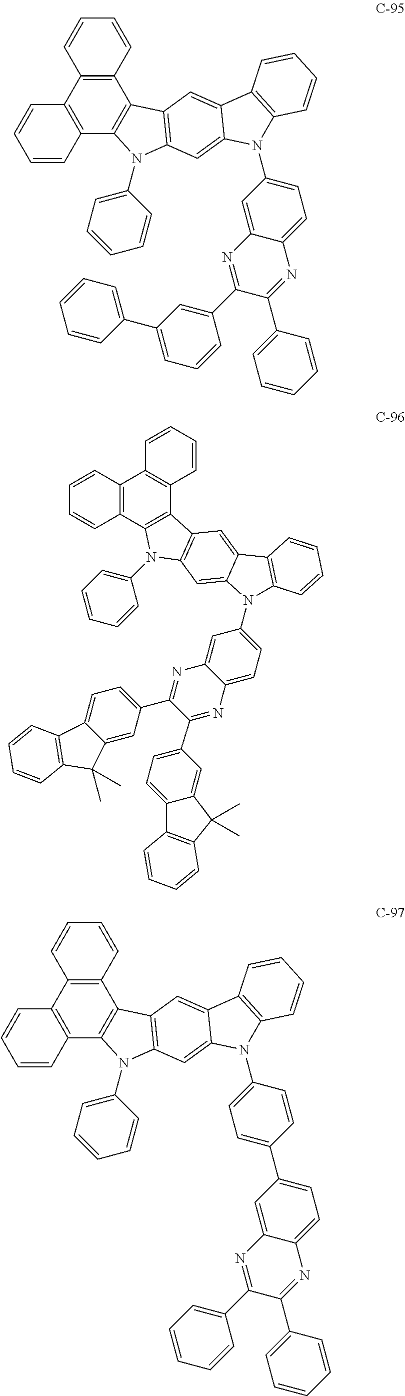 [SMILES: c1ccc(-c2cccc(-c3nc4cc(-n5c6ccccc6c6cc7c8c9ccccc9c9ccccc9c8n(-c8ccccc8)c7cc65)ccc4nc3-c3ccccc3)c2)cc1 ; CC1(C)c2ccccc2-c2ccc(-c3nc4ccc(-n5c6ccccc6c6cc7c8c9ccccc9c9ccccc9c8n(-c8ccccc8)c7cc65)cc4nc3-c3ccc4c(c3)C(C)(C)c3ccccc3-4)cc21 ; c1ccc(-c2nc3ccc(-c4ccc(-n5c6ccccc6c6cc7c8c9ccccc9c9ccccc9c8n(-c8ccccc8)c7cc65)cc4)cc3nc2-c2ccccc2)cc1]

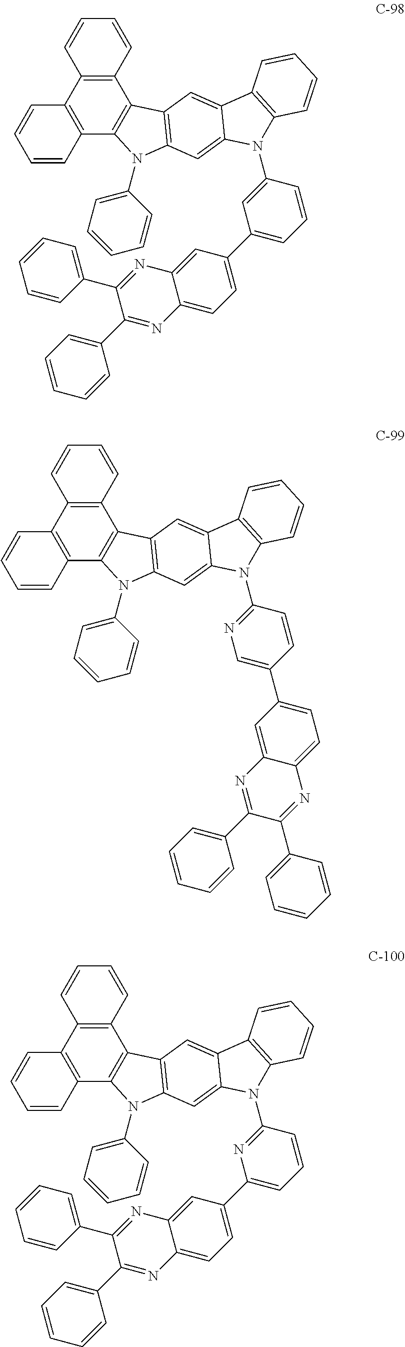 [SMILES: c1ccc(-c2nc3ccc(-c4cccc(-n5c6ccccc6c6cc7c8c9ccccc9c9ccccc9c8n(-c8ccccc8)c7cc65)c4)cc3nc2-c2ccccc2)cc1 ; c1ccc(-c2nc3ccc(-c4ccc(-n5c6ccccc6c6cc7c8c9ccccc9c9ccccc9c8n(-c8ccccc8)c7cc65)nc4)cc3nc2-c2ccccc2)cc1 ; c1ccc(-c2nc3ccc(-c4cccc(-n5c6ccccc6c6cc7c8c9ccccc9c9ccccc9c8n(-c8ccccc8)c7cc65)n4)cc3nc2-c2ccccc2)cc1]

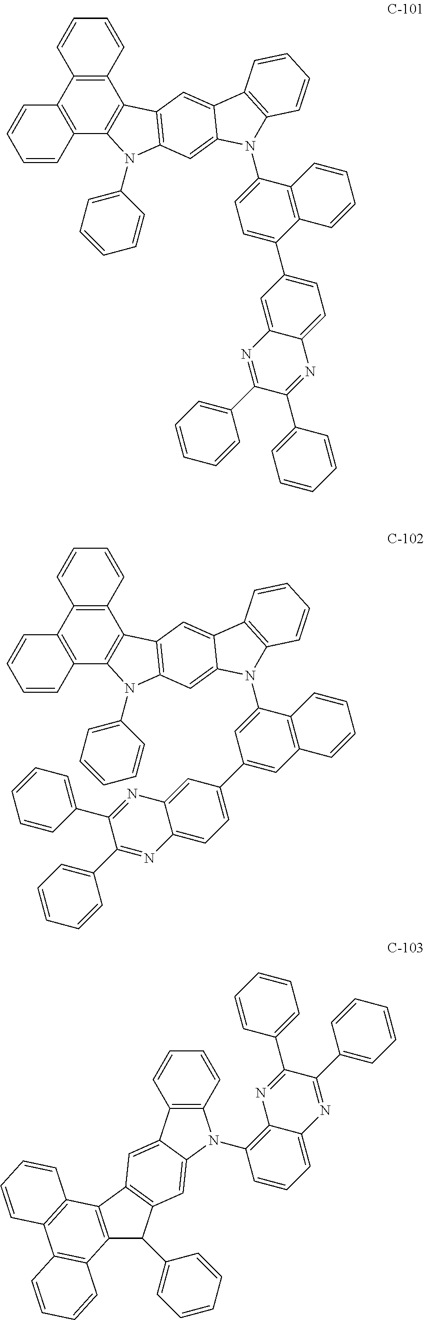 [SMILES: c1ccc(-c2nc3ccc(-c4ccc(-n5c6ccccc6c6cc7c8c9ccccc9c9ccccc9c8n(-c8ccccc8)c7cc65)c5ccccc45)cc3nc2-c2ccccc2)cc1 ; c1ccc(-c2nc3ccc(-c4cc(-n5c6ccccc6c6cc7c8c9ccccc9c9ccccc9c8n(-c8ccccc8)c7cc65)c5ccccc5c4)cc3nc2-c2ccccc2)cc1 ; c1ccc(-c2nc3cccc(-n4c5ccccc5c5cc6c(cc54)C(c4ccccc4)c4c-6c5ccccc5c5ccccc45)c3nc2-c2ccccc2)cc1]

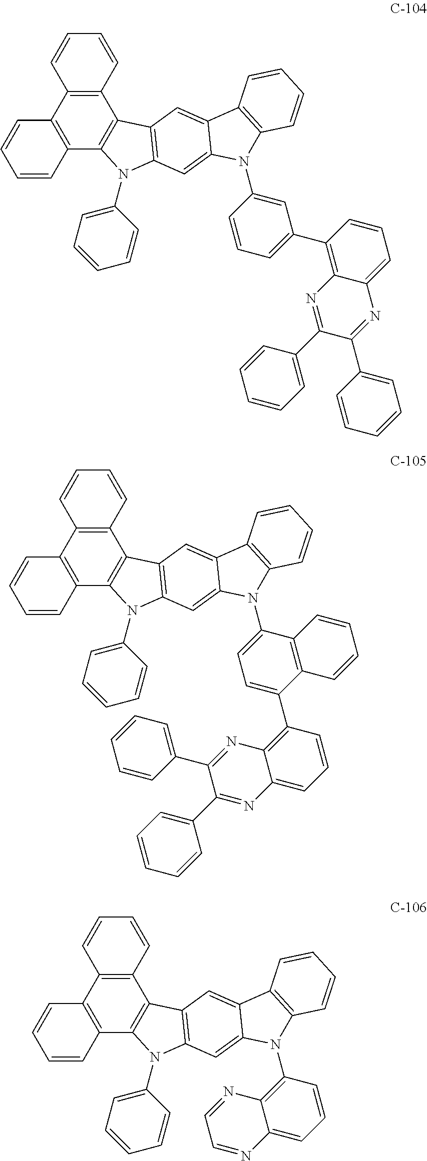 [SMILES: c1ccc(-c2nc3cccc(-c4cccc(-n5c6ccccc6c6cc7c8c9ccccc9c9ccccc9c8n(-c8ccccc8)c7cc65)c4)c3nc2-c2ccccc2)cc1 ; c1ccc(-c2nc3cccc(-c4ccc(-n5c6ccccc6c6cc7c8c9ccccc9c9ccccc9c8n(-c8ccccc8)c7cc65)c5ccccc45)c3nc2-c2ccccc2)cc1 ; c1ccc(-n2c3cc4c(cc3c3c5ccccc5c5ccccc5c32)c2ccccc2n4-c2cccc3nccnc23)cc1]

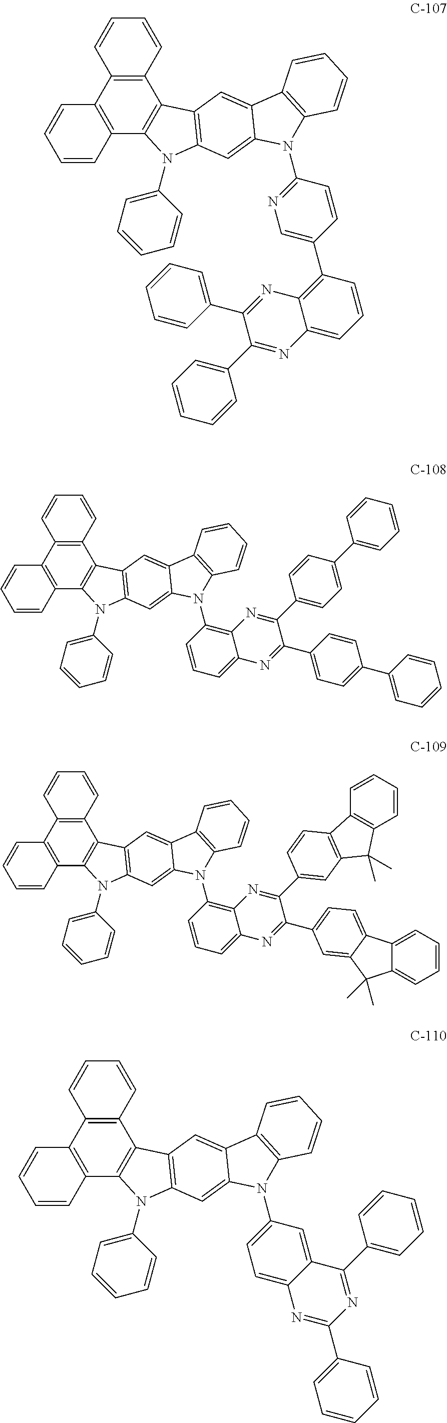 [SMILES: c1ccc(-c2nc3cccc(-c4ccc(-n5c6ccccc6c6cc7c8c9ccccc9c9ccccc9c8n(-c8ccccc8)c7cc65)nc4)c3nc2-c2ccccc2)cc1 ; c1ccc(-c2ccc(-c3nc4cccc(-n5c6ccccc6c6cc7c8c9ccccc9c9ccccc9c8n(-c8ccccc8)c7cc65)c4nc3-c3ccc(-c4ccccc4)cc3)cc2)cc1 ; CC1(C)c2ccccc2-c2ccc(-c3nc4cccc(-n5c6ccccc6c6cc7c8c9ccccc9c9ccccc9c8n(-c8ccccc8)c7cc65)c4nc3-c3ccc4c(c3)C(C)(C)c3ccccc3-4)cc21 ; c1ccc(-c2nc(-c3ccccc3)c3cc(-n4c5ccccc5c5cc6c7c8ccccc8c8ccccc8c7n(-c7ccccc7)c6cc54)ccc3n2)cc1]

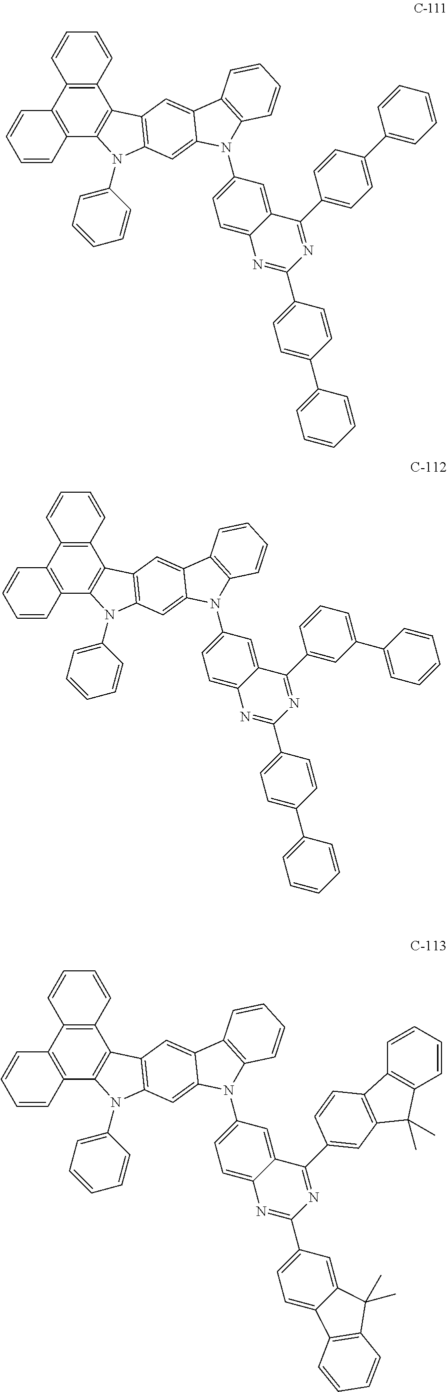 [SMILES: c1ccc(-c2ccc(-c3nc(-c4ccc(-c5ccccc5)cc4)c4cc(-n5c6ccccc6c6cc7c8c9ccccc9c9ccccc9c8n(-c8ccccc8)c7cc65)ccc4n3)cc2)cc1 ; c1ccc(-c2ccc(-c3nc(-c4cccc(-c5ccccc5)c4)c4cc(-n5c6ccccc6c6cc7c8c9ccccc9c9ccccc9c8n(-c8ccccc8)c7cc65)ccc4n3)cc2)cc1 ; CC1(C)c2ccccc2-c2ccc(-c3nc(-c4ccc5c(c4)C(C)(C)c4ccccc4-5)c4cc(-n5c6ccccc6c6cc7c8c9ccccc9c9ccccc9c8n(-c8ccccc8)c7cc65)ccc4n3)cc21]

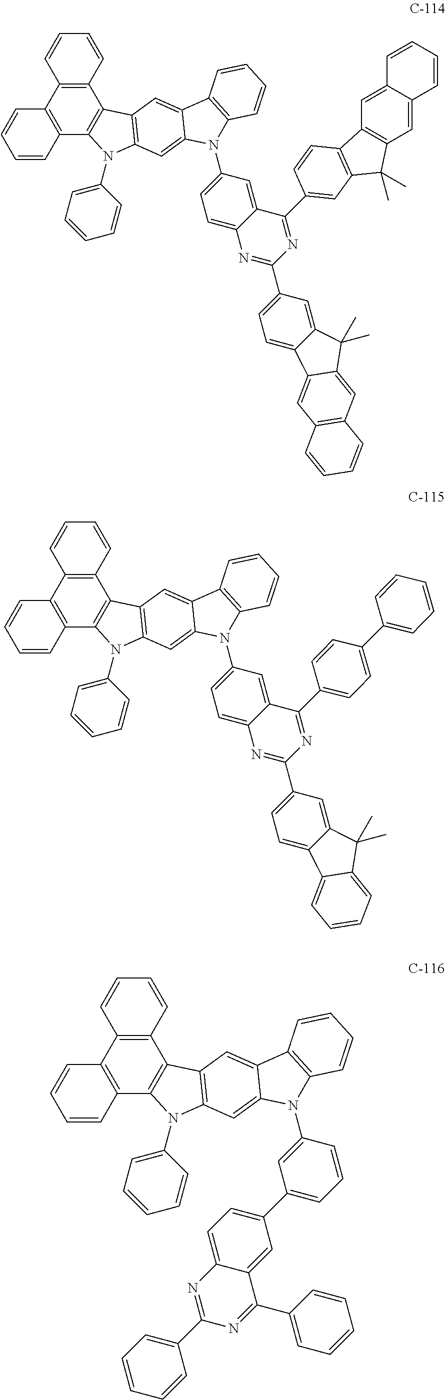 [SMILES: CC1(C)c2cc(-c3nc(-c4ccc5c(c4)C(C)(C)c4cc6ccccc6cc4-5)c4cc(-n5c6ccccc6c6cc7c8c9ccccc9c9ccccc9c8n(-c8ccccc8)c7cc65)ccc4n3)ccc2-c2cc3ccccc3cc21 ; CC1(C)c2ccccc2-c2ccc(-c3nc(-c4ccc(-c5ccccc5)cc4)c4cc(-n5c6ccccc6c6cc7c8c9ccccc9c9ccccc9c8n(-c8ccccc8)c7cc65)ccc4n3)cc21 ; c1ccc(-c2nc(-c3ccccc3)c3cc(-c4cccc(-n5c6ccccc6c6cc7c8c9ccccc9c9ccccc9c8n(-c8ccccc8)c7cc65)c4)ccc3n2)cc1]

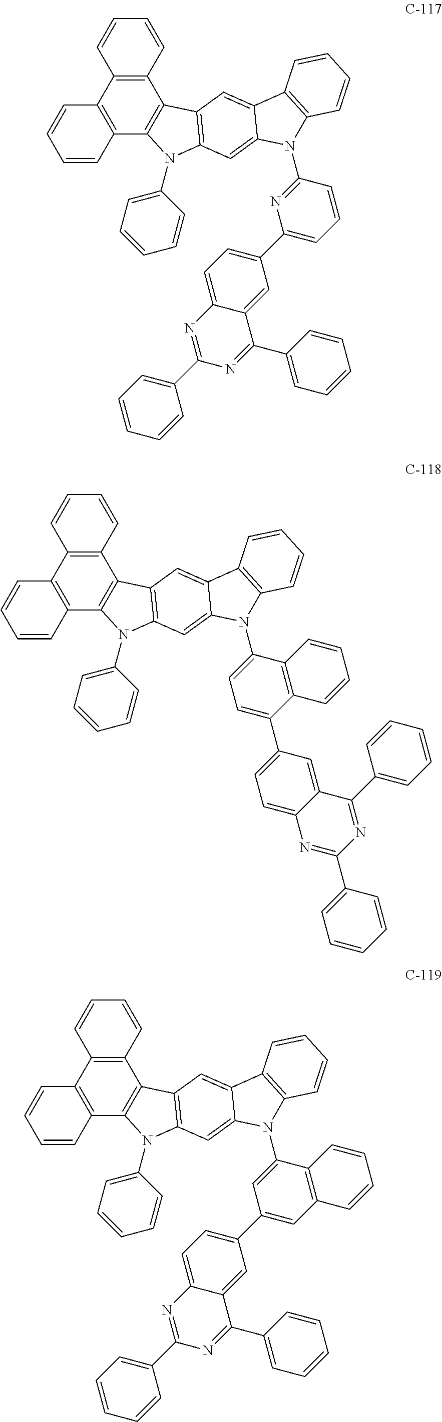 [SMILES: c1ccc(-c2nc(-c3ccccc3)c3cc(-c4cccc(-n5c6ccccc6c6cc7c8c9ccccc9c9ccccc9c8n(-c8ccccc8)c7cc65)n4)ccc3n2)cc1 ; c1ccc(-c2nc(-c3ccccc3)c3cc(-c4ccc(-n5c6ccccc6c6cc7c8c9ccccc9c9ccccc9c8n(-c8ccccc8)c7cc65)c5ccccc45)ccc3n2)cc1 ; c1ccc(-c2nc(-c3ccccc3)c3cc(-c4cc(-n5c6ccccc6c6cc7c8c9ccccc9c9ccccc9c8n(-c8ccccc8)c7cc65)c5ccccc5c4)ccc3n2)cc1]

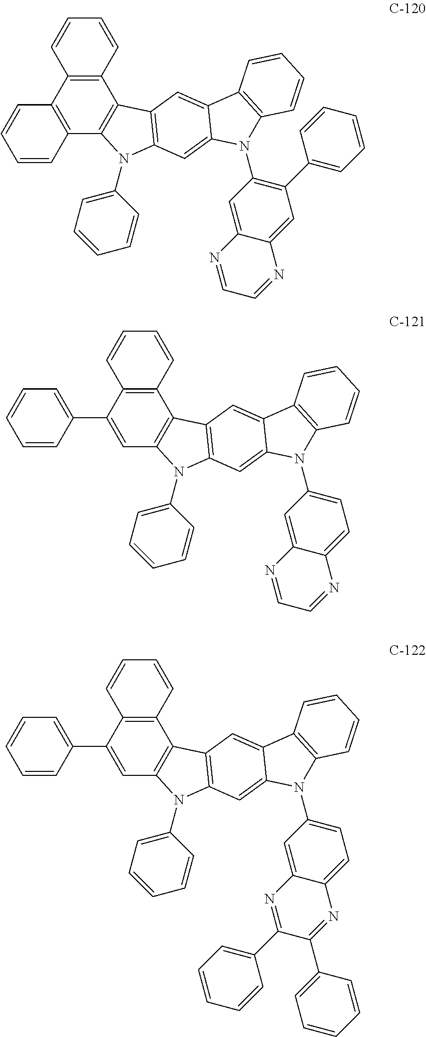 [SMILES: c1ccc(-c2cc3nccnc3cc2-n2c3ccccc3c3cc4c5c6ccccc6c6ccccc6c5n(-c5ccccc5)c4cc32)cc1 ; c1ccc(-c2cc3c(c4ccccc24)c2cc4c5ccccc5n(-c5ccc6nccnc6c5)c4cc2n3-c2ccccc2)cc1 ; c1ccc(-c2nc3ccc(-n4c5ccccc5c5cc6c7c8ccccc8c(-c8ccccc8)cc7n(-c7ccccc7)c6cc54)cc3nc2-c2ccccc2)cc1]

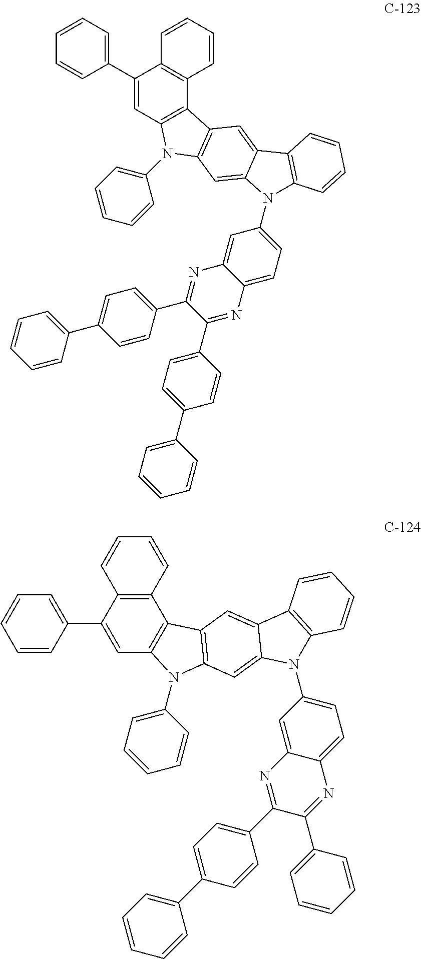

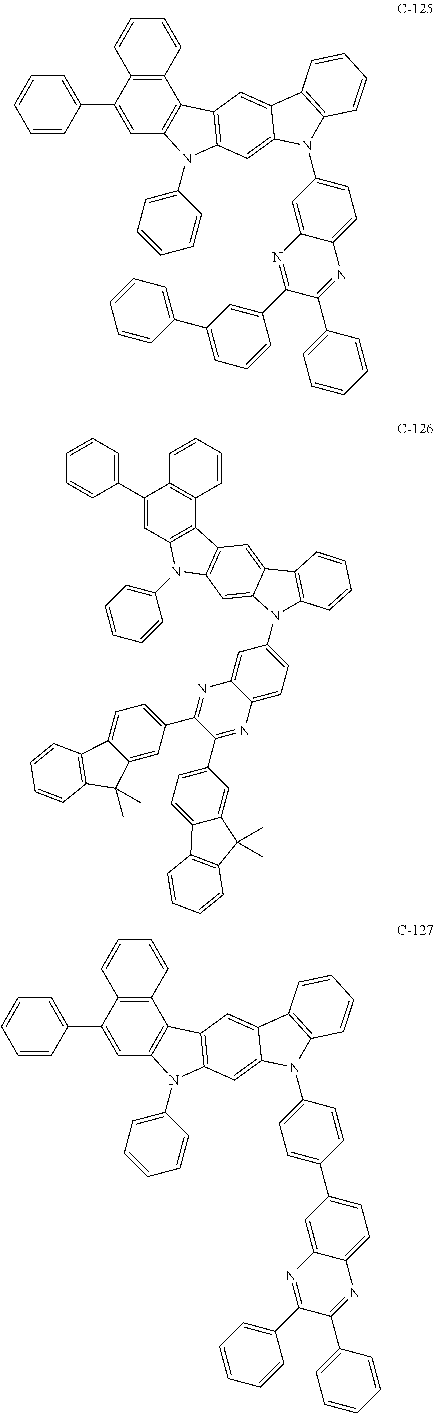

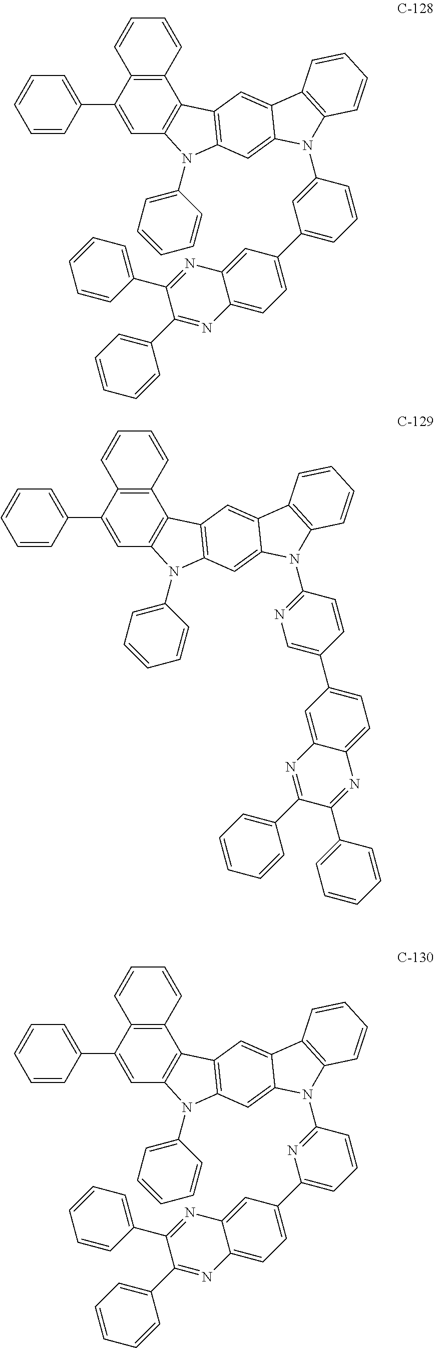

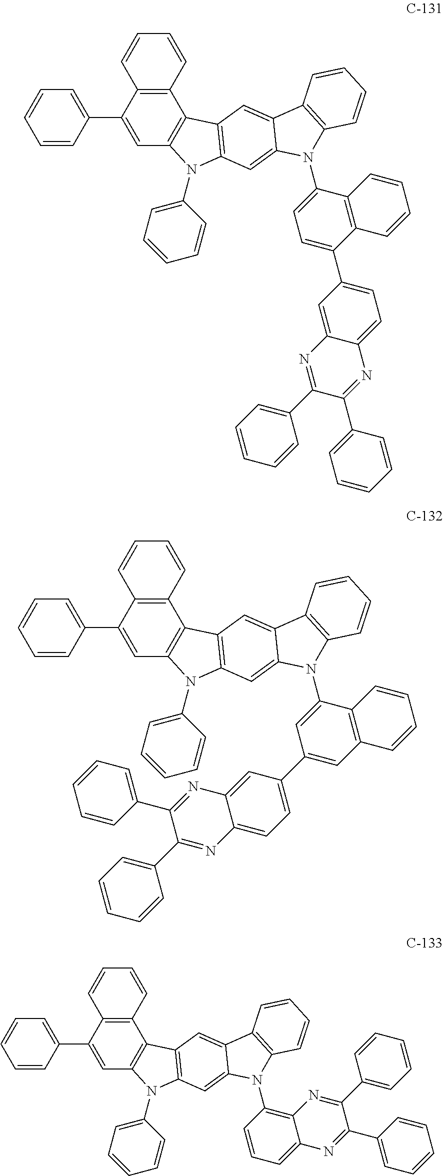 [SMILES: c1ccc(-c2nc3ccc(-c4ccc(-n5c6ccccc6c6cc7c8c9ccccc9c(-c9ccccc9)cc8n(-c8ccccc8)c7cc65)c5ccccc45)cc3nc2-c2ccccc2)cc1 ; c1ccc(-c2nc3ccc(-c4cc(-n5c6ccccc6c6cc7c8c9ccccc9c(-c9ccccc9)cc8n(-c8ccccc8)c7cc65)c5ccccc5c4)cc3nc2-c2ccccc2)cc1 ; c1ccc(-c2nc3cccc(-n4c5ccccc5c5cc6c7c8ccccc8c(-c8ccccc8)cc7n(-c7ccccc7)c6cc54)c3nc2-c2ccccc2)cc1]

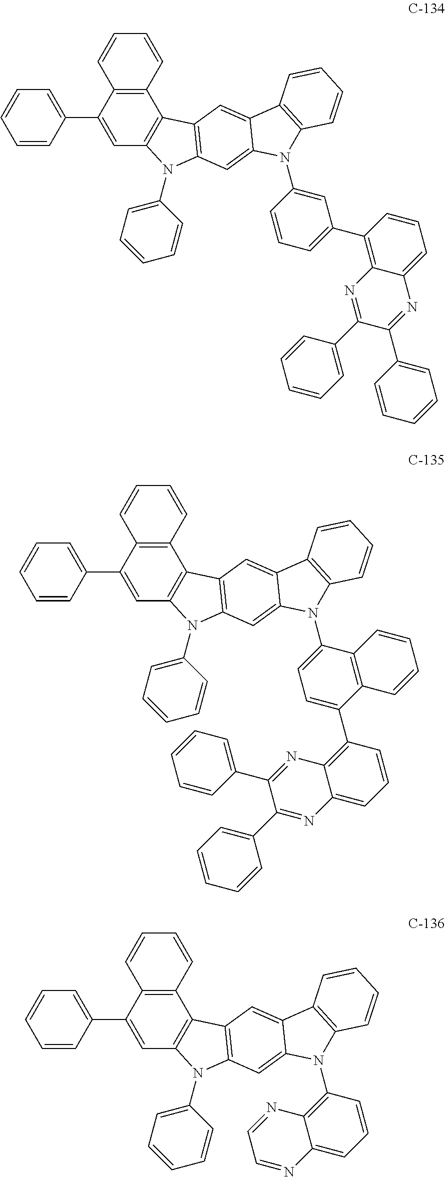 [SMILES: c1ccc(-c2nc3cccc(-c4cccc(-n5c6ccccc6c6cc7c8c9ccccc9c(-c9ccccc9)cc8n(-c8ccccc8)c7cc65)c4)c3nc2-c2ccccc2)cc1 ; c1ccc(-c2nc3cccc(-c4ccc(-n5c6ccccc6c6cc7c8c9ccccc9c(-c9ccccc9)cc8n(-c8ccccc8)c7cc65)c5ccccc45)c3nc2-c2ccccc2)cc1 ; c1ccc(-c2cc3c(c4ccccc24)c2cc4c5ccccc5n(-c5cccc6nccnc56)c4cc2n3-c2ccccc2)cc1]

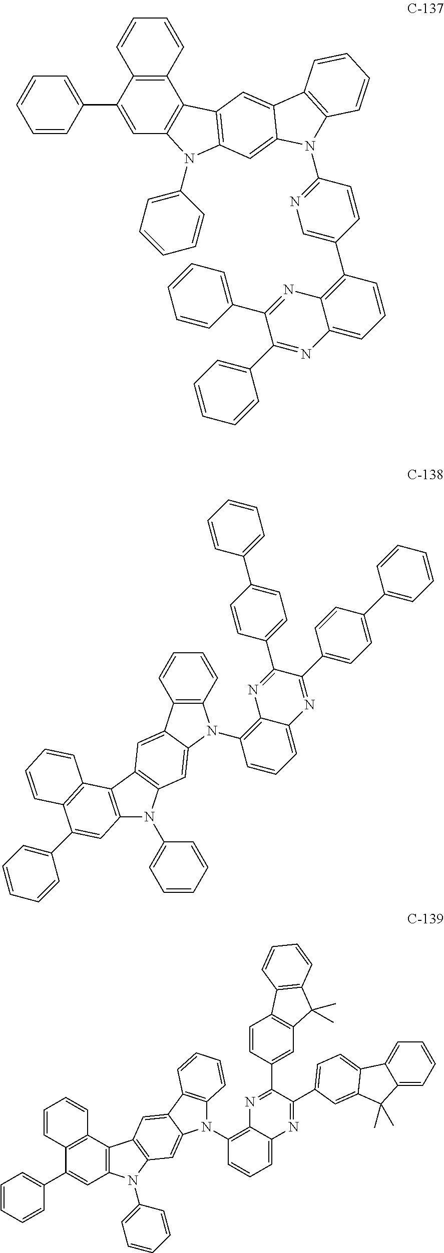 [SMILES: c1ccc(-c2nc3cccc(-c4ccc(-n5c6ccccc6c6cc7c8c9ccccc9c(-c9ccccc9)cc8n(-c8ccccc8)c7cc65)nc4)c3nc2-c2ccccc2)cc1 ; c1ccc(-c2ccc(-c3nc4cccc(-n5c6ccccc6c6cc7c8c9ccccc9c(-c9ccccc9)cc8n(-c8ccccc8)c7cc65)c4nc3-c3ccc(-c4ccccc4)cc3)cc2)cc1 ; CC1(C)c2ccccc2-c2ccc(-c3nc4cccc(-n5c6ccccc6c6cc7c8c9ccccc9c(-c9ccccc9)cc8n(-c8ccccc8)c7cc65)c4nc3-c3ccc4c(c3)C(C)(C)c3ccccc3-4)cc21]

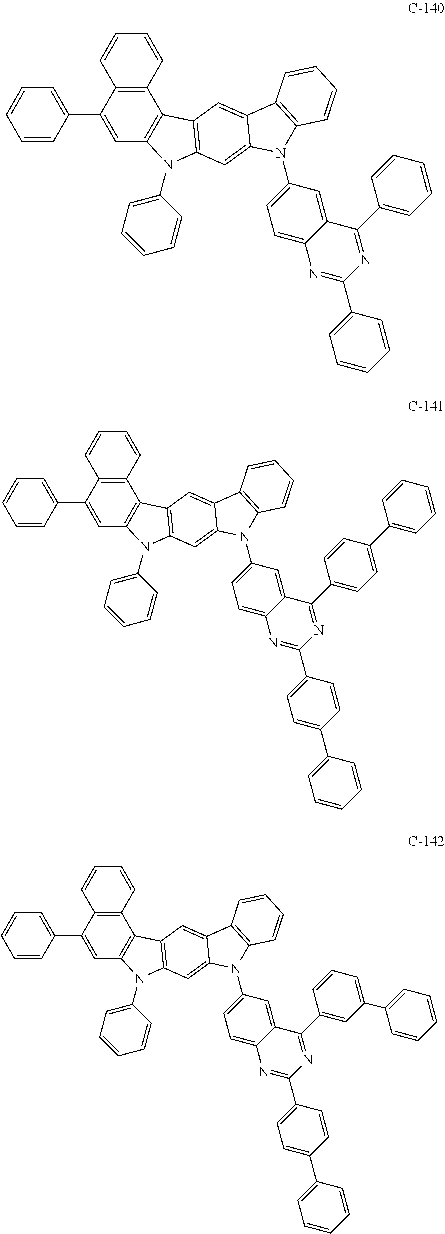 [SMILES: c1ccc(-c2nc(-c3ccccc3)c3cc(-n4c5ccccc5c5cc6c7c8ccccc8c(-c8ccccc8)cc7n(-c7ccccc7)c6cc54)ccc3n2)cc1 ; c1ccc(-c2ccc(-c3nc(-c4ccc(-c5ccccc5)cc4)c4cc(-n5c6ccccc6c6cc7c8c9ccccc9c(-c9ccccc9)cc8n(-c8ccccc8)c7cc65)ccc4n3)cc2)cc1 ; c1ccc(-c2ccc(-c3nc(-c4cccc(-c5ccccc5)c4)c4cc(-n5c6ccccc6c6cc7c8c9ccccc9c(-c9ccccc9)cc8n(-c8ccccc8)c7cc65)ccc4n3)cc2)cc1]

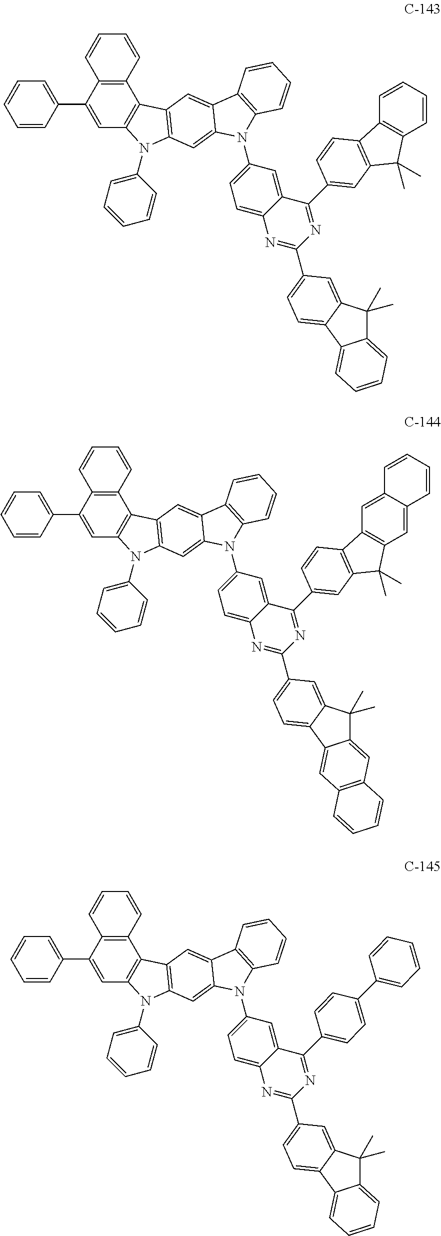 [SMILES: CC1(C)c2ccccc2-c2ccc(-c3nc(-c4ccc5c(c4)C(C)(C)c4ccccc4-5)c4cc(-n5c6ccccc6c6cc7c8c9ccccc9c(-c9ccccc9)cc8n(-c8ccccc8)c7cc65)ccc4n3)cc21 ; CC1(C)c2cc(-c3nc(-c4ccc5c(c4)C(C)(C)c4cc6ccccc6cc4-5)c4cc(-n5c6ccccc6c6cc7c8c9ccccc9c(-c9ccccc9)cc8n(-c8ccccc8)c7cc65)ccc4n3)ccc2-c2cc3ccccc3cc21 ; CC1(C)c2ccccc2-c2ccc(-c3nc(-c4ccc(-c5ccccc5)cc4)c4cc(-n5c6ccccc6c6cc7c8c9ccccc9c(-c9ccccc9)cc8n(-c8ccccc8)c7cc65)ccc4n3)cc21]

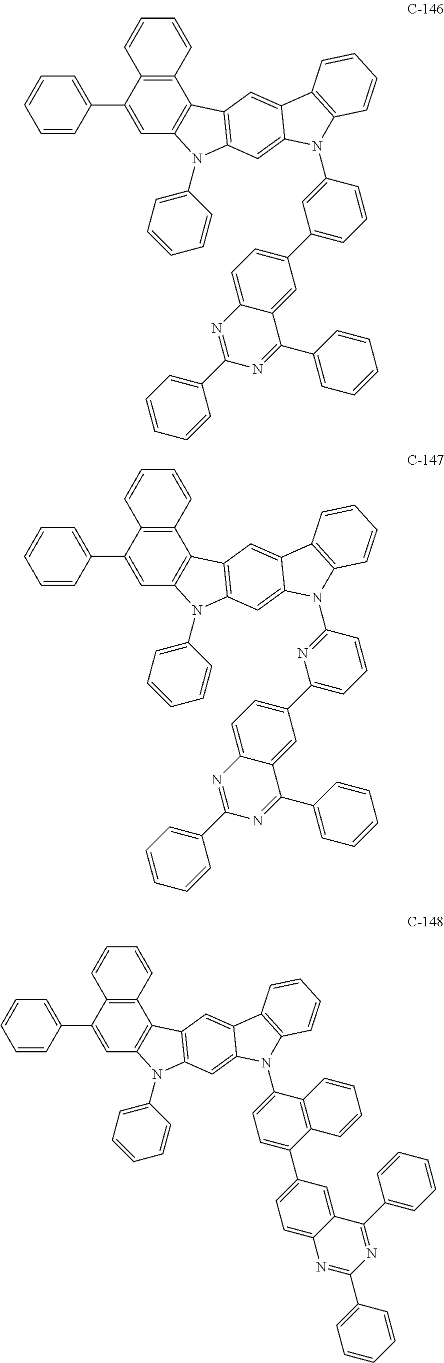

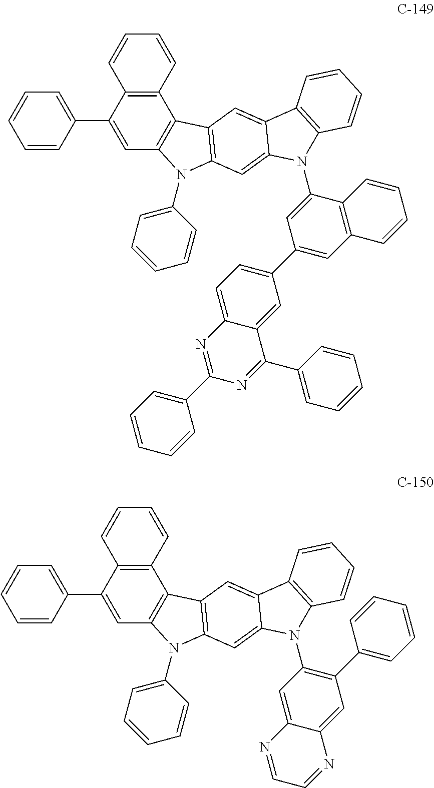 [SMILES: c1ccc(-c2nc(-c3ccccc3)c3cc(-c4cc(-n5c6ccccc6c6cc7c8c9ccccc9c(-c9ccccc9)cc8n(-c8ccccc8)c7cc65)c5ccccc5c4)ccc3n2)cc1 ; c1ccc(-c2cc3nccnc3cc2-n2c3ccccc3c3cc4c5c6ccccc6c(-c6ccccc6)cc5n(-c5ccccc5)c4cc32)cc1]

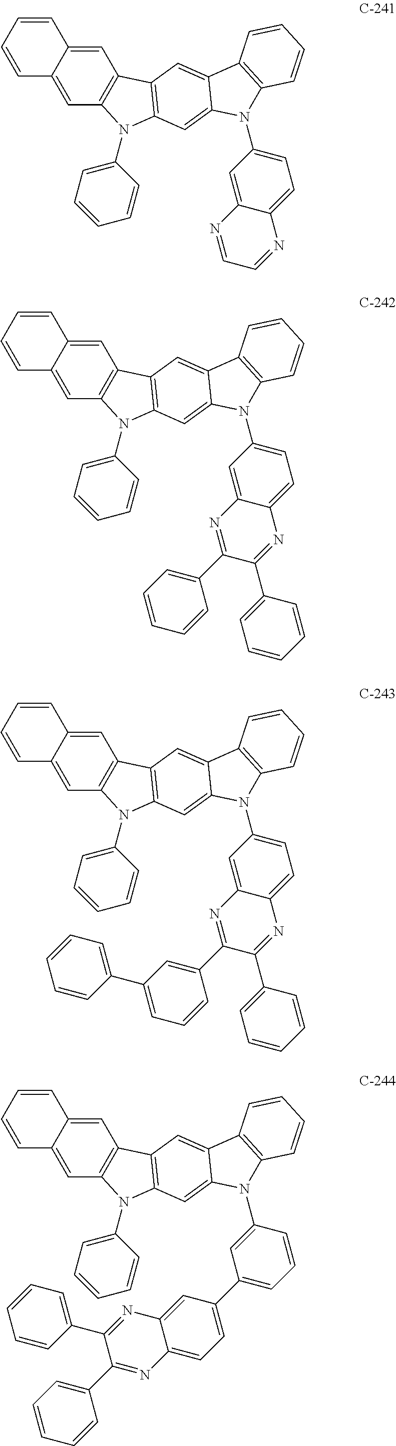

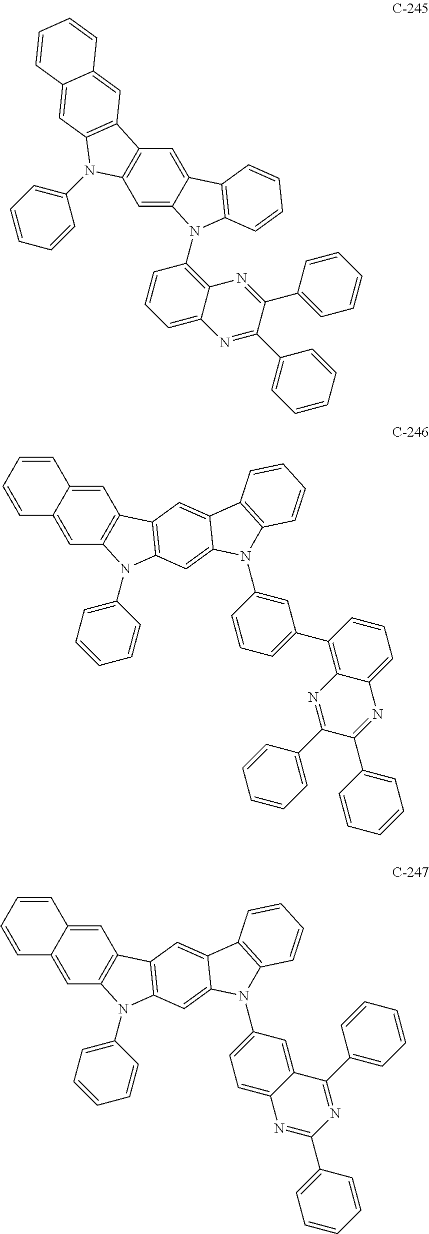 [SMILES: c1ccc(-c2nc3cccc(-n4c5ccccc5c5cc6c7cc8ccccc8cc7n(-c7ccccc7)c6cc54)c3nc2-c2ccccc2)cc1 ; c1ccc(-c2nc3cccc(-c4cccc(-n5c6ccccc6c6cc7c8cc9ccccc9cc8n(-c8ccccc8)c7cc65)c4)c3nc2-c2ccccc2)cc1 ; c1ccc(-c2nc(-c3ccccc3)c3cc(-n4c5ccccc5c5cc6c7cc8ccccc8cc7n(-c7ccccc7)c6cc54)ccc3n2)cc1]

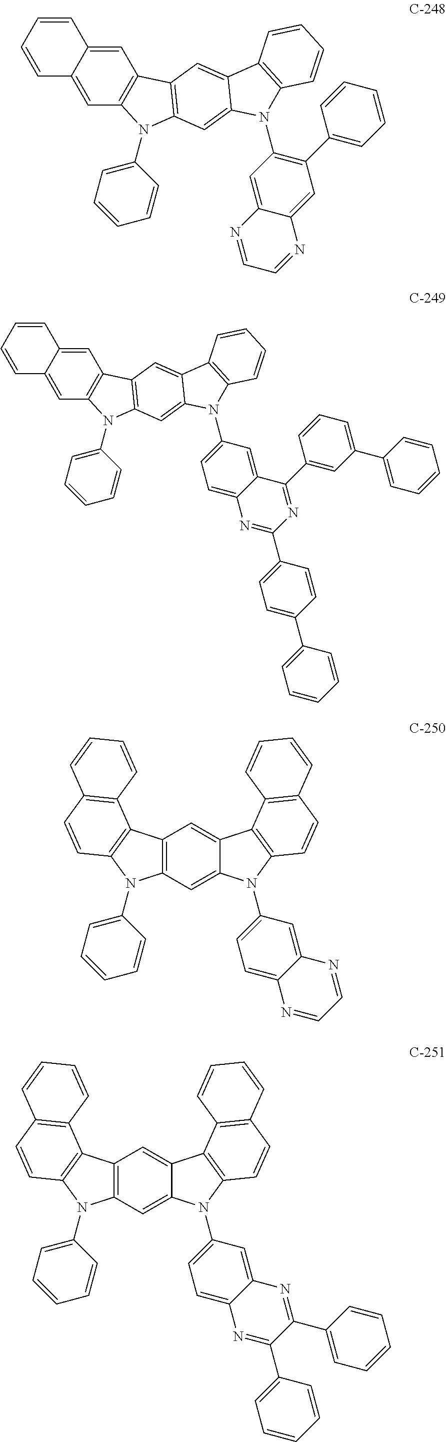

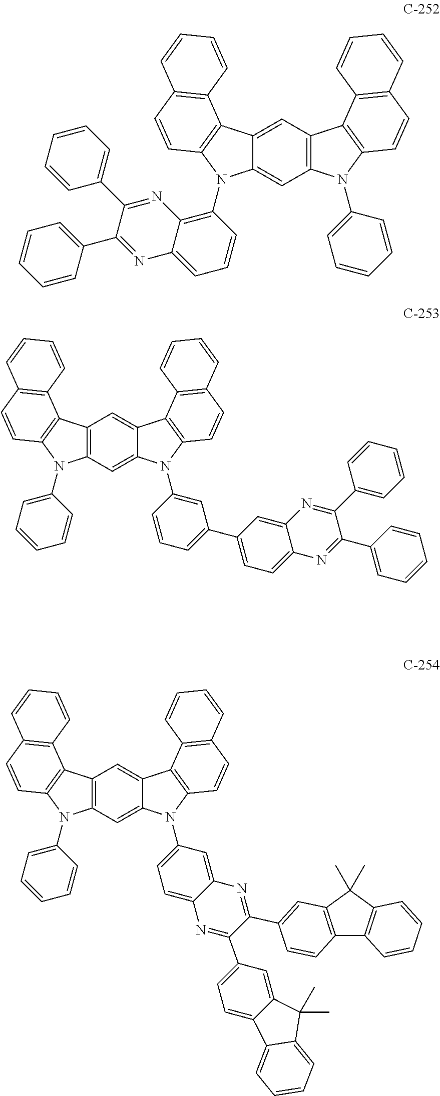 [SMILES: c1ccc(-c2nc3cccc(-n4c5cc6c(cc5c5c7ccccc7ccc54)c4c5ccccc5ccc4n6-c4ccccc4)c3nc2-c2ccccc2)cc1 ; c1ccc(-c2nc3ccc(-c4cccc(-n5c6cc7c(cc6c6c8ccccc8ccc65)c5c6ccccc6ccc5n7-c5ccccc5)c4)cc3nc2-c2ccccc2)cc1 ; CC1(C)c2ccccc2-c2ccc(-c3nc4ccc(-n5c6cc7c(cc6c6c8ccccc8ccc65)c5c6ccccc6ccc5n7-c5ccccc5)cc4nc3-c3ccc4c(c3)C(C)(C)c3ccccc3-4)cc21]

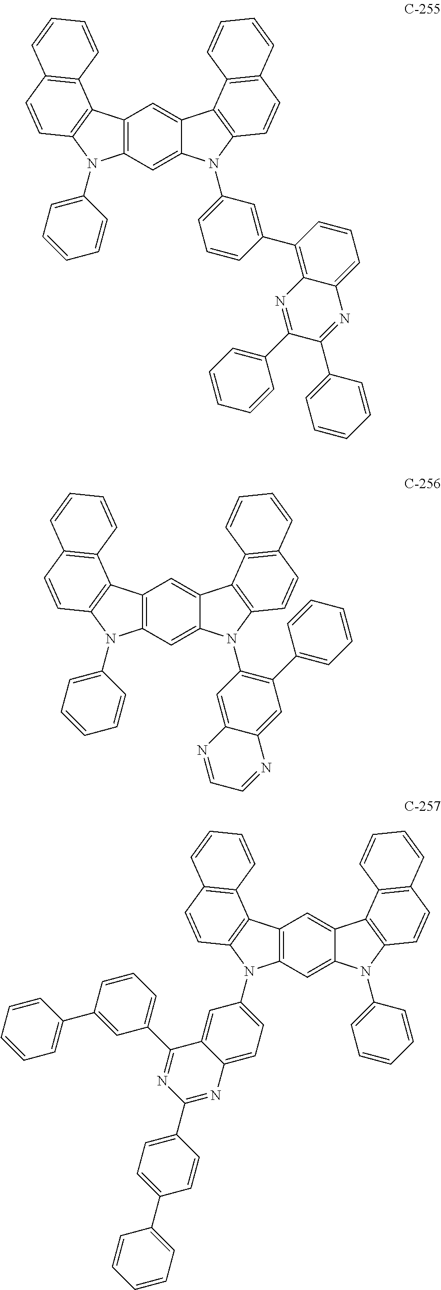

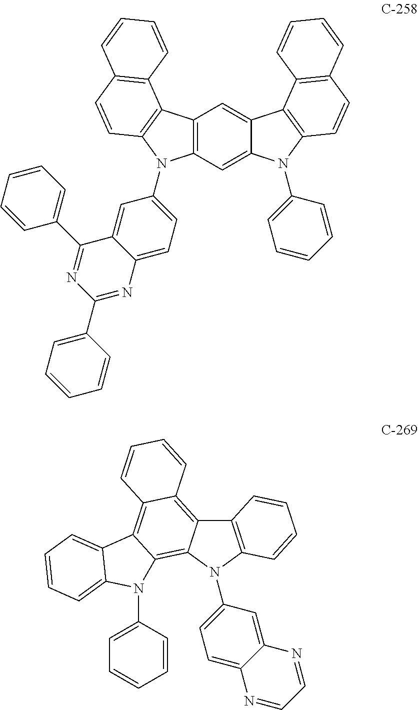 [SMILES: c1ccc(-c2nc(-c3ccccc3)c3cc(-n4c5cc6c(cc5c5c7ccccc7ccc54)c4c5ccccc5ccc4n6-c4ccccc4)ccc3n2)cc1 ; c1ccc(-n2c3ccccc3c3c4ccccc4c4c5ccccc5n(-c5ccc6nccnc6c5)c4c32)cc1]

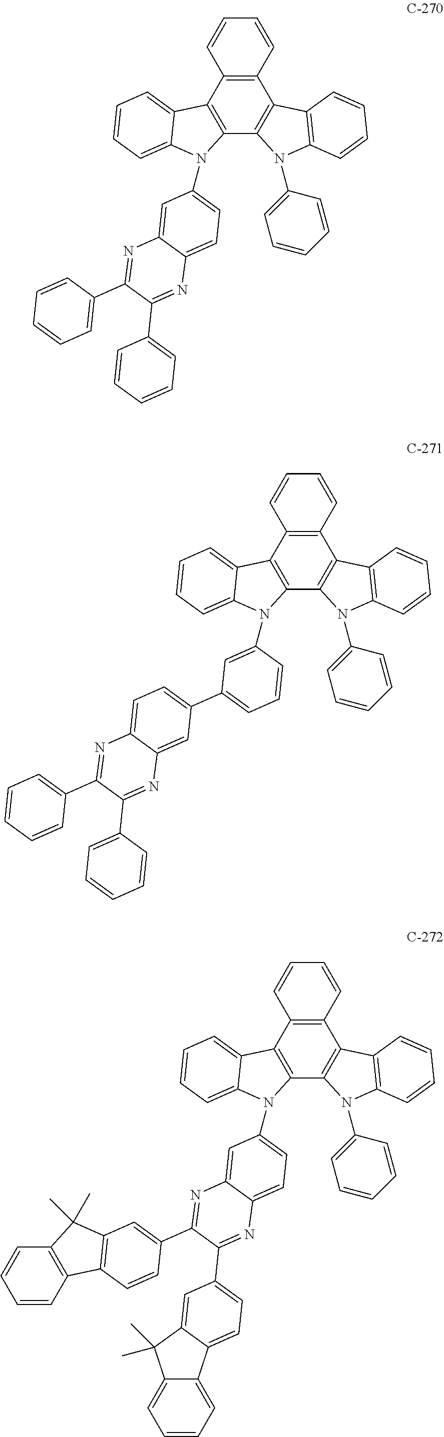 [SMILES: c1ccc(-c2nc3ccc(-n4c5ccccc5c5c6ccccc6c6c7ccccc7n(-c7ccccc7)c6c54)cc3nc2-c2ccccc2)cc1 ; c1ccc(-c2nc3ccc(-c4cccc(-n5c6ccccc6c6c7ccccc7c7c8ccccc8n(-c8ccccc8)c7c65)c4)cc3nc2-c2ccccc2)cc1 ; CC1(C)c2ccccc2-c2ccc(-c3nc4ccc(-n5c6ccccc6c6c7ccccc7c7c8ccccc8n(-c8ccccc8)c7c65)cc4nc3-c3ccc4c(c3)C(C)(C)c3ccccc3-4)cc21]

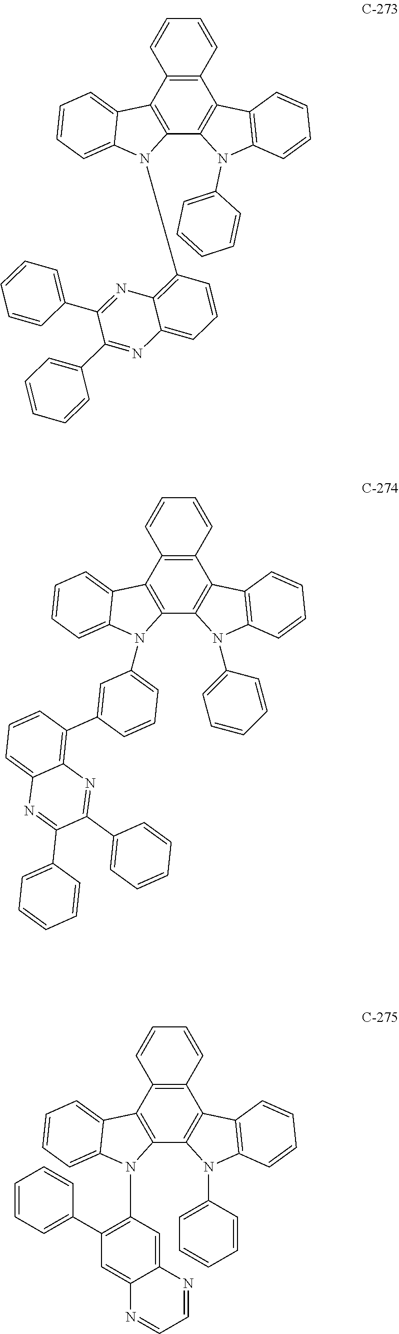 [SMILES: c1ccc(-c2nc3cccc(-n4c5ccccc5c5c6ccccc6c6c7ccccc7n(-c7ccccc7)c6c54)c3nc2-c2ccccc2)cc1 ; c1ccc(-c2nc3cccc(-c4cccc(-n5c6ccccc6c6c7ccccc7c7c8ccccc8n(-c8ccccc8)c7c65)c4)c3nc2-c2ccccc2)cc1 ; c1ccc(-c2cc3nccnc3cc2-n2c3ccccc3c3c4ccccc4c4c5ccccc5n(-c5ccccc5)c4c32)cc1]

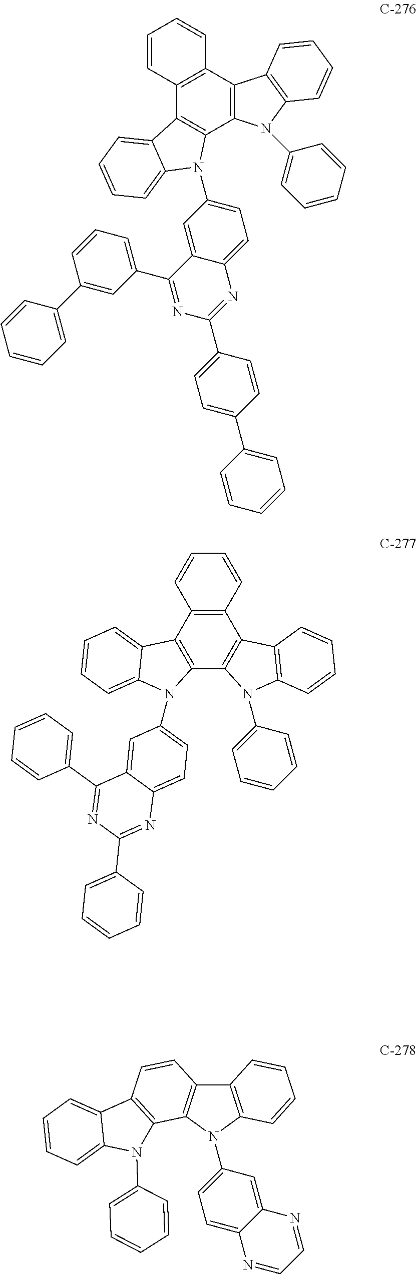 [SMILES: c1ccc(-c2ccc(-c3nc(-c4cccc(-c5ccccc5)c4)c4cc(-n5c6ccccc6c6c7ccccc7c7c8ccccc8n(-c8ccccc8)c7c65)ccc4n3)cc2)cc1 ; c1ccc(-c2nc(-c3ccccc3)c3cc(-n4c5ccccc5c5c6ccccc6c6c7ccccc7n(-c7ccccc7)c6c54)ccc3n2)cc1 ; c1ccc(-n2c3ccccc3c3ccc4c5ccccc5n(-c5ccc6nccnc6c5)c4c32)cc1]

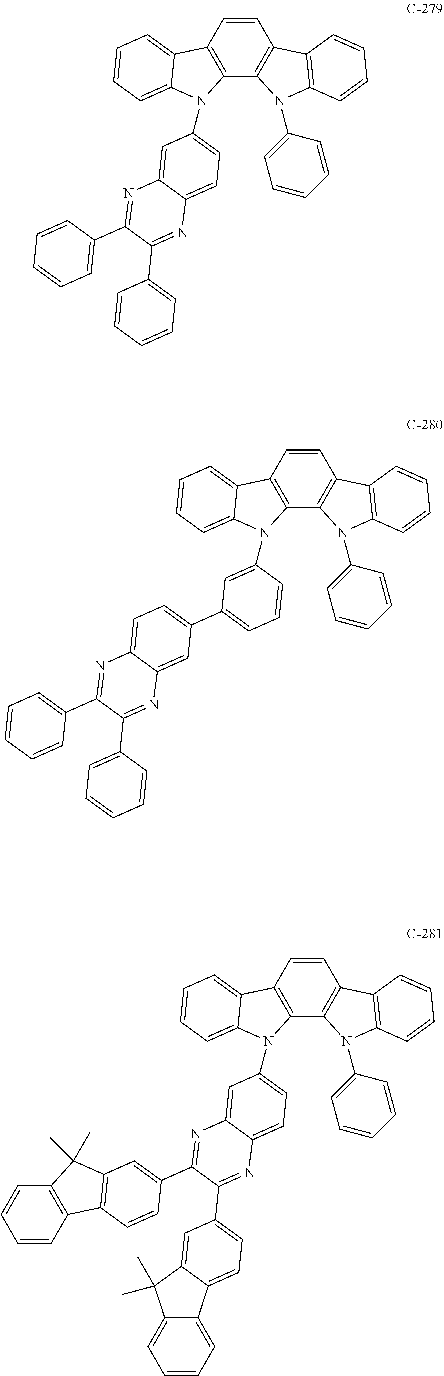 [SMILES: c1ccc(-c2nc3ccc(-n4c5ccccc5c5ccc6c7ccccc7n(-c7ccccc7)c6c54)cc3nc2-c2ccccc2)cc1 ; c1ccc(-c2nc3ccc(-c4cccc(-n5c6ccccc6c6ccc7c8ccccc8n(-c8ccccc8)c7c65)c4)cc3nc2-c2ccccc2)cc1 ; CC1(C)c2ccccc2-c2ccc(-c3nc4ccc(-n5c6ccccc6c6ccc7c8ccccc8n(-c8ccccc8)c7c65)cc4nc3-c3ccc4c(c3)C(C)(C)c3ccccc3-4)cc21]

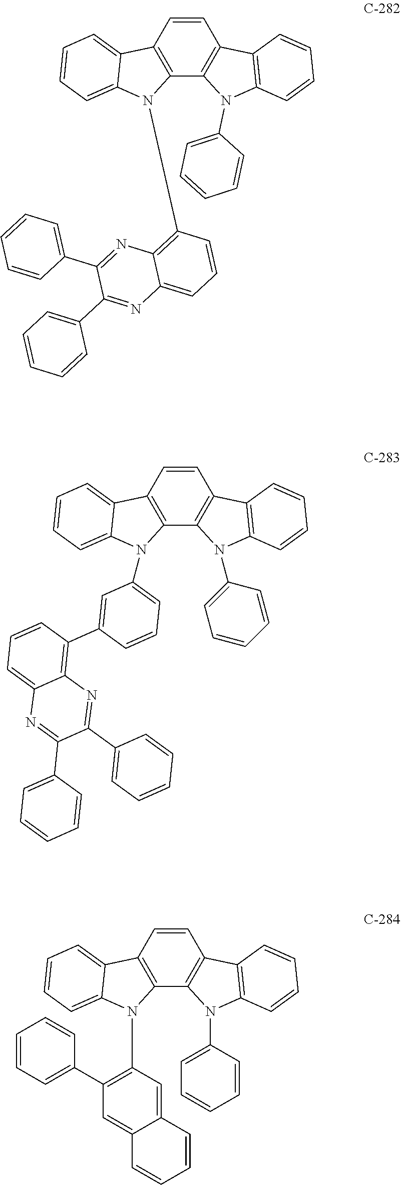 [SMILES: c1ccc(-c2nc3cccc(-n4c5ccccc5c5ccc6c7ccccc7n(-c7ccccc7)c6c54)c3nc2-c2ccccc2)cc1 ; c1ccc(-c2nc3cccc(-c4cccc(-n5c6ccccc6c6ccc7c8ccccc8n(-c8ccccc8)c7c65)c4)c3nc2-c2ccccc2)cc1 ; c1ccc(-c2cc3ccccc3cc2-n2c3ccccc3c3ccc4c5ccccc5n(-c5ccccc5)c4c32)cc1]

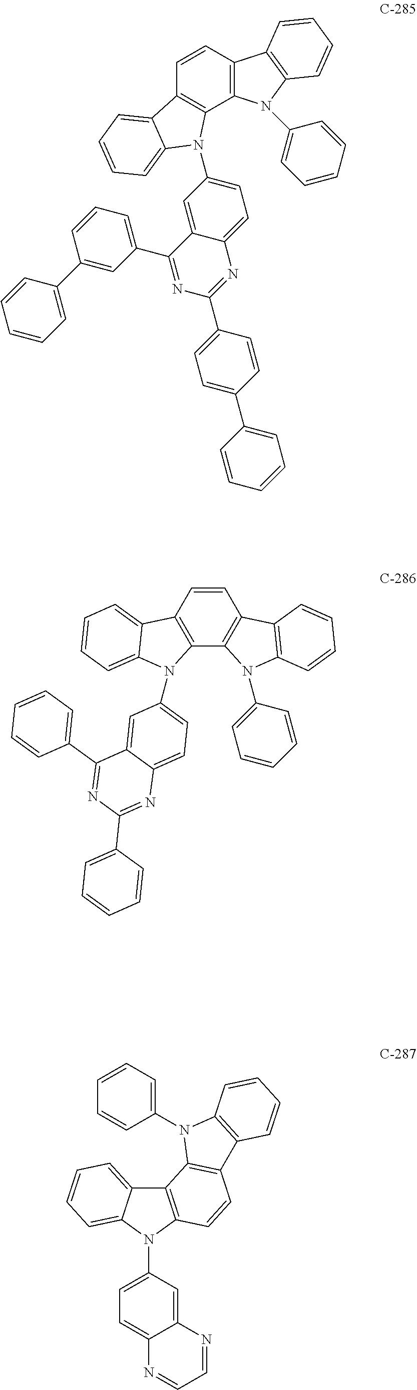 [SMILES: c1ccc(-c2ccc(-c3nc(-c4cccc(-c5ccccc5)c4)c4cc(-n5c6ccccc6c6ccc7c8ccccc8n(-c8ccccc8)c7c65)ccc4n3)cc2)cc1 ; c1ccc(-c2nc(-c3ccccc3)c3cc(-n4c5ccccc5c5ccc6c7ccccc7n(-c7ccccc7)c6c54)ccc3n2)cc1 ; c1ccc(-n2c3ccccc3c3ccc4c(c5ccccc5n4-c4ccc5nccnc5c4)c32)cc1]

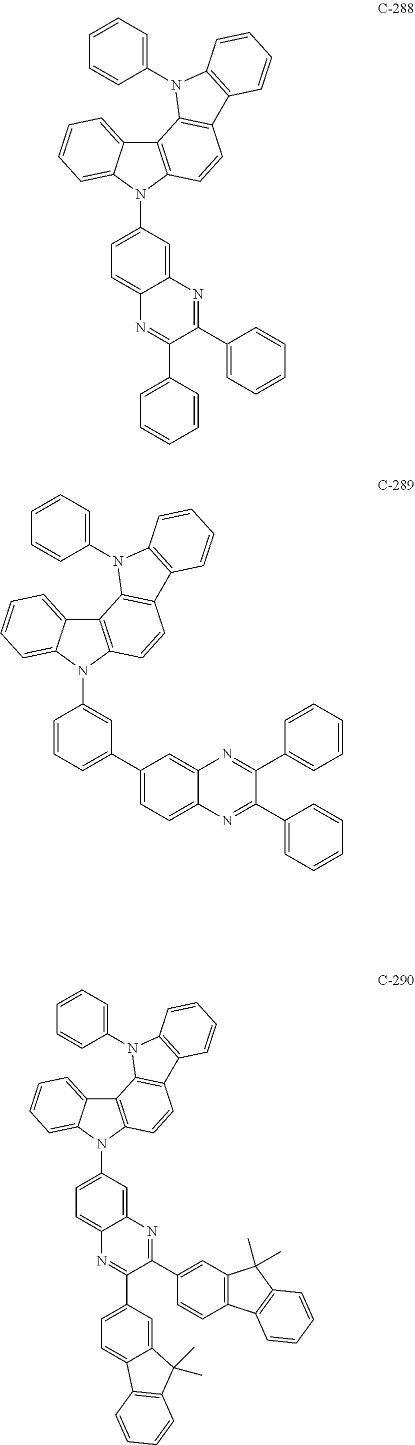 [SMILES: c1ccc(-c2nc3ccc(-n4c5ccccc5c5c4ccc4c6ccccc6n(-c6ccccc6)c45)cc3nc2-c2ccccc2)cc1 ; c1ccc(-c2nc3ccc(-c4cccc(-n5c6ccccc6c6c5ccc5c7ccccc7n(-c7ccccc7)c56)c4)cc3nc2-c2ccccc2)cc1 ; CC1(C)c2ccccc2-c2ccc(-c3nc4ccc(-n5c6ccccc6c6c5ccc5c7ccccc7n(-c7ccccc7)c56)cc4nc3-c3ccc4c(c3)C(C)(C)c3ccccc3-4)cc21]

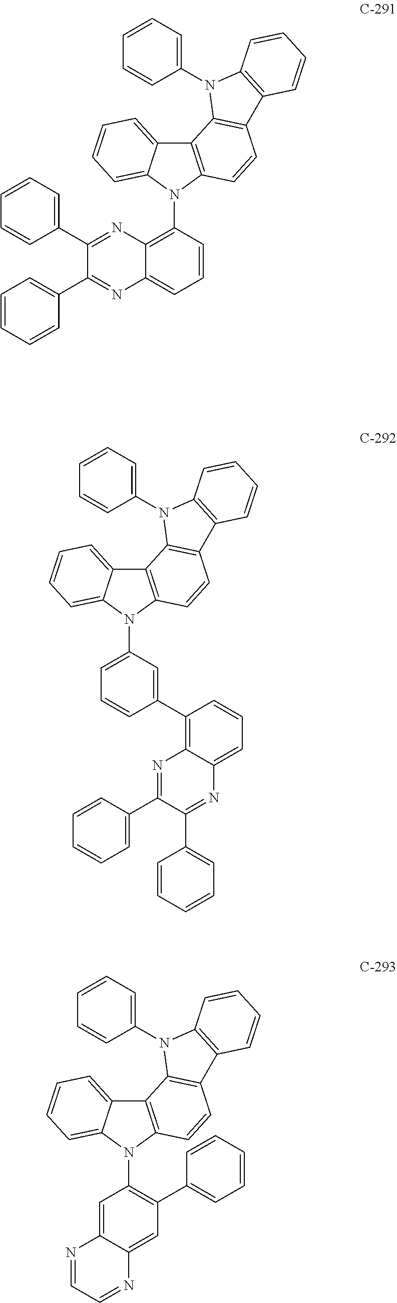 [SMILES: c1ccc(-c2nc3cccc(-n4c5ccccc5c5c4ccc4c6ccccc6n(-c6ccccc6)c45)c3nc2-c2ccccc2)cc1 ; c1ccc(-c2nc3cccc(-c4cccc(-n5c6ccccc6c6c5ccc5c7ccccc7n(-c7ccccc7)c56)c4)c3nc2-c2ccccc2)cc1 ; c1ccc(-c2cc3nccnc3cc2-n2c3ccccc3c3c2ccc2c4ccccc4n(-c4ccccc4)c23)cc1]

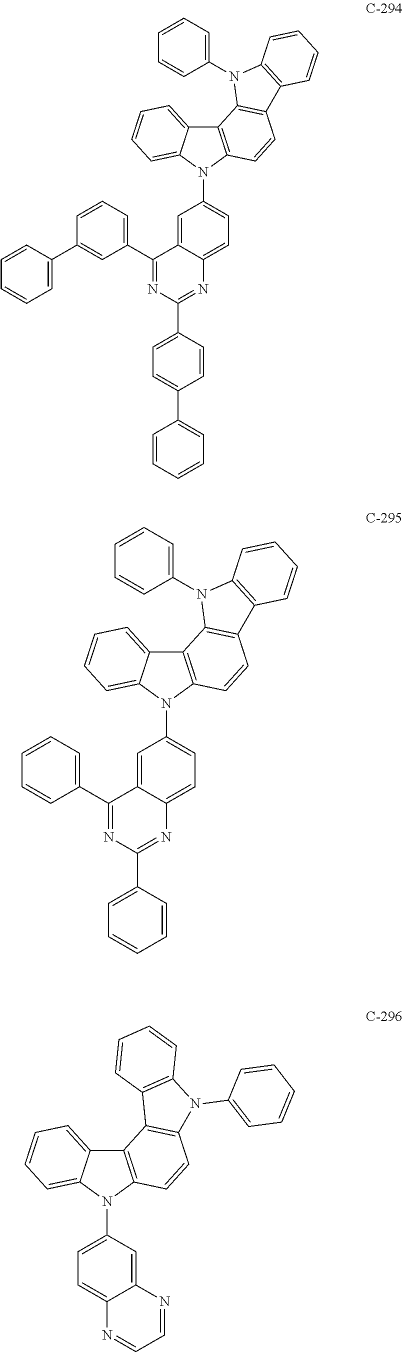 [SMILES: c1ccc(-c2ccc(-c3nc(-c4cccc(-c5ccccc5)c4)c4cc(-n5c6ccccc6c6c5ccc5c7ccccc7n(-c7ccccc7)c56)ccc4n3)cc2)cc1 ; c1ccc(-c2nc(-c3ccccc3)c3cc(-n4c5ccccc5c5c4ccc4c6ccccc6n(-c6ccccc6)c45)ccc3n2)cc1 ; c1ccc(-n2c3ccccc3c3c4c5ccccc5n(-c5ccc6nccnc6c5)c4ccc32)cc1]

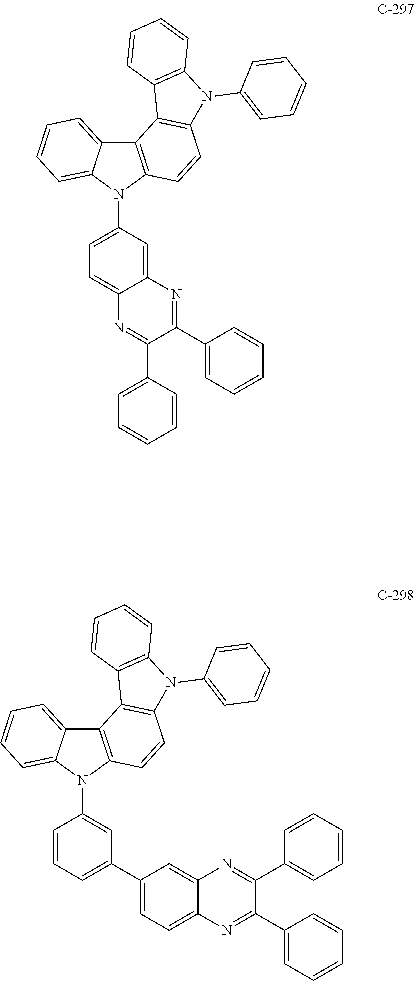 [SMILES: c1ccc(-c2nc3ccc(-n4c5ccccc5c5c6c7ccccc7n(-c7ccccc7)c6ccc54)cc3nc2-c2ccccc2)cc1 ; c1ccc(-c2nc3ccc(-c4cccc(-n5c6ccccc6c6c7c8ccccc8n(-c8ccccc8)c7ccc65)c4)cc3nc2-c2ccccc2)cc1]

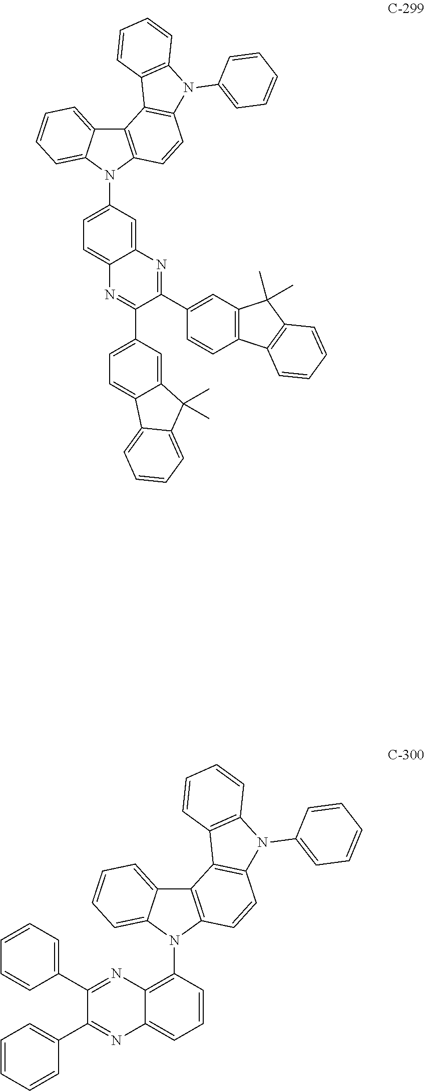 [SMILES: CC1(C)c2ccccc2-c2ccc(-c3nc4ccc(-n5c6ccccc6c6c7c8ccccc8n(-c8ccccc8)c7ccc65)cc4nc3-c3ccc4c(c3)C(C)(C)c3ccccc3-4)cc21 ; c1ccc(-c2nc3cccc(-n4c5ccccc5c5c6c7ccccc7n(-c7ccccc7)c6ccc54)c3nc2-c2ccccc2)cc1]

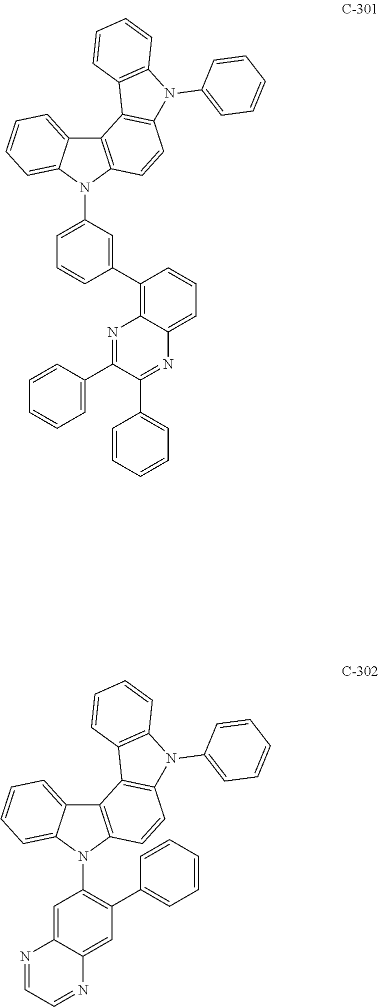 [SMILES: c1ccc(-c2nc3cccc(-c4cccc(-n5c6ccccc6c6c7c8ccccc8n(-c8ccccc8)c7ccc65)c4)c3nc2-c2ccccc2)cc1 ; c1ccc(-c2cc3nccnc3cc2-n2c3ccccc3c3c4c5ccccc5n(-c5ccccc5)c4ccc32)cc1]

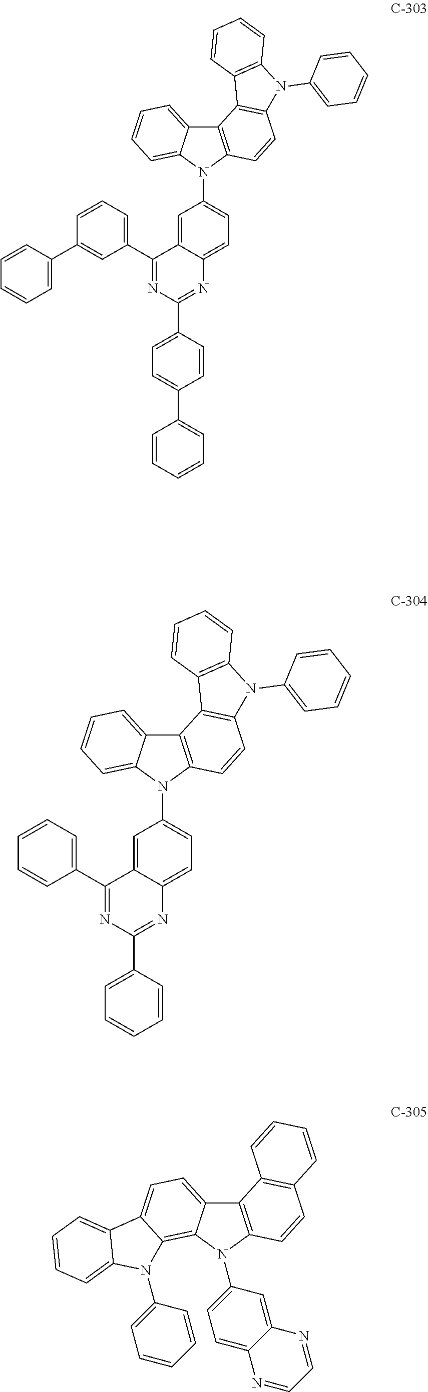 [SMILES: c1ccc(-c2ccc(-c3nc(-c4cccc(-c5ccccc5)c4)c4cc(-n5c6ccccc6c6c7c8ccccc8n(-c8ccccc8)c7ccc65)ccc4n3)cc2)cc1 ; c1ccc(-c2nc(-c3ccccc3)c3cc(-n4c5ccccc5c5c6c7ccccc7n(-c7ccccc7)c6ccc54)ccc3n2)cc1 ; c1ccc(-n2c3ccccc3c3ccc4c5c6ccccc6ccc5n(-c5ccc6nccnc6c5)c4c32)cc1]

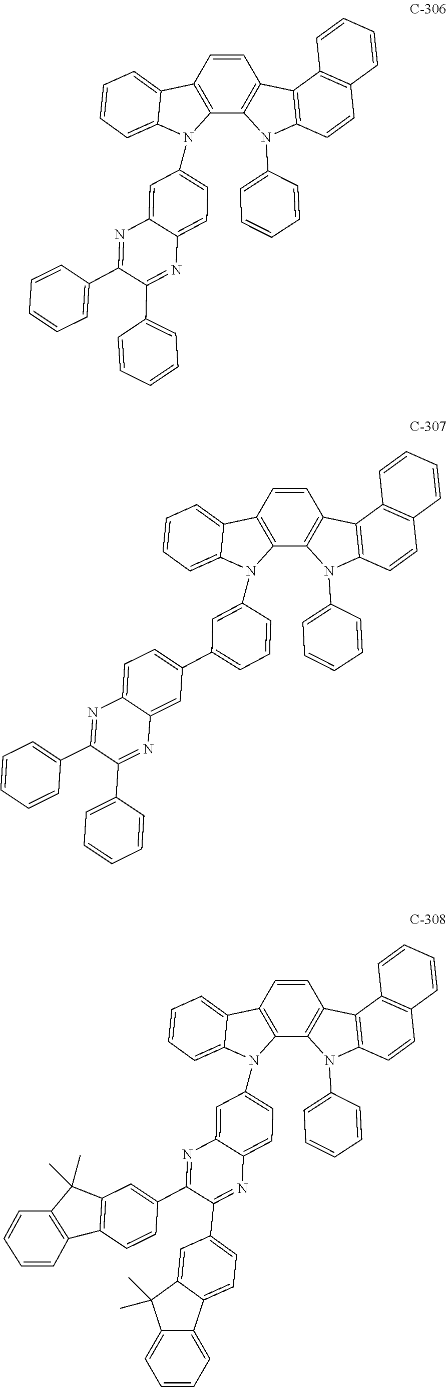 [SMILES: c1ccc(-c2nc3ccc(-n4c5ccccc5c5ccc6c7c8ccccc8ccc7n(-c7ccccc7)c6c54)cc3nc2-c2ccccc2)cc1 ; c1ccc(-c2nc3ccc(-c4cccc(-n5c6ccccc6c6ccc7c8c9ccccc9ccc8n(-c8ccccc8)c7c65)c4)cc3nc2-c2ccccc2)cc1 ; CC1(C)c2ccccc2-c2ccc(-c3nc4ccc(-n5c6ccccc6c6ccc7c8c9ccccc9ccc8n(-c8ccccc8)c7c65)cc4nc3-c3ccc4c(c3)C(C)(C)c3ccccc3-4)cc21]

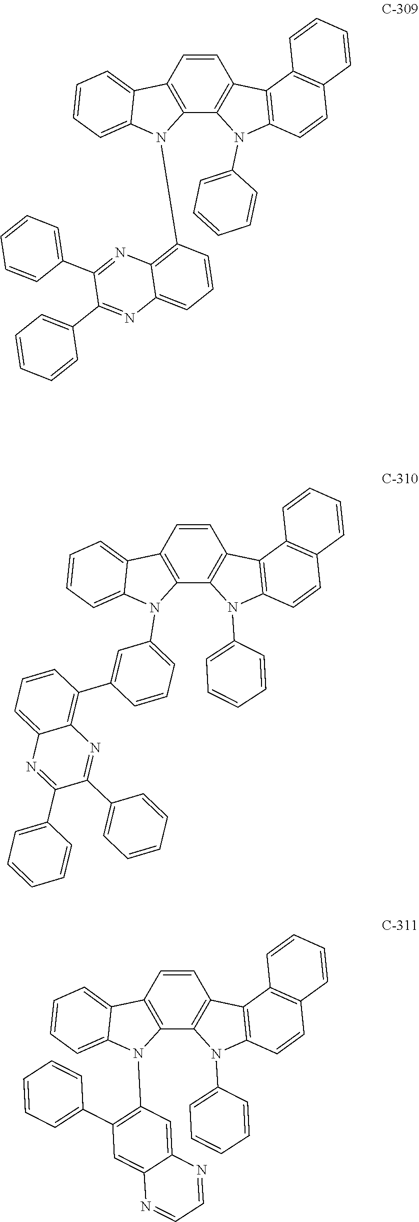 [SMILES: c1ccc(-c2nc3cccc(-n4c5ccccc5c5ccc6c7c8ccccc8ccc7n(-c7ccccc7)c6c54)c3nc2-c2ccccc2)cc1 ; c1ccc(-c2nc3cccc(-c4cccc(-n5c6ccccc6c6ccc7c8c9ccccc9ccc8n(-c8ccccc8)c7c65)c4)c3nc2-c2ccccc2)cc1 ; c1ccc(-c2cc3nccnc3cc2-n2c3ccccc3c3ccc4c5c6ccccc6ccc5n(-c5ccccc5)c4c32)cc1]

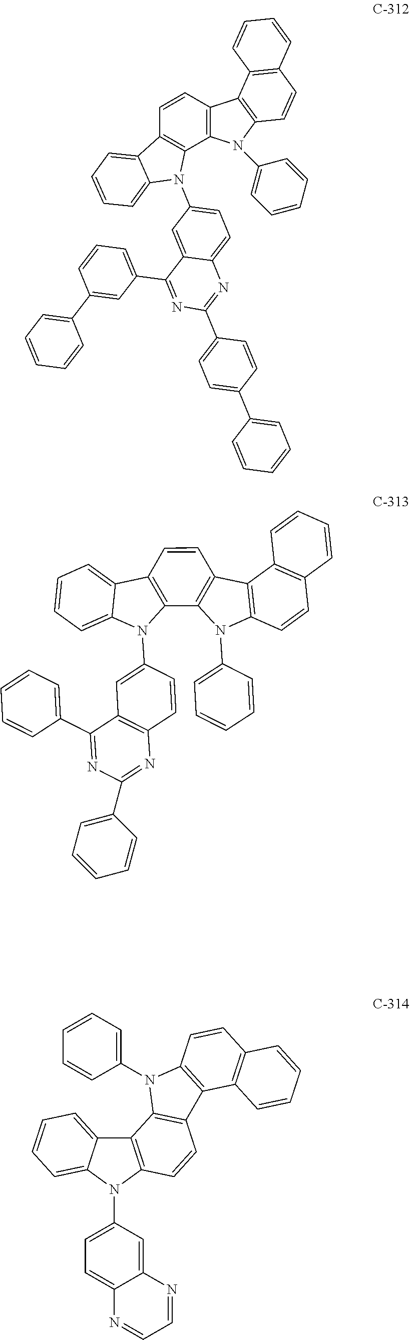

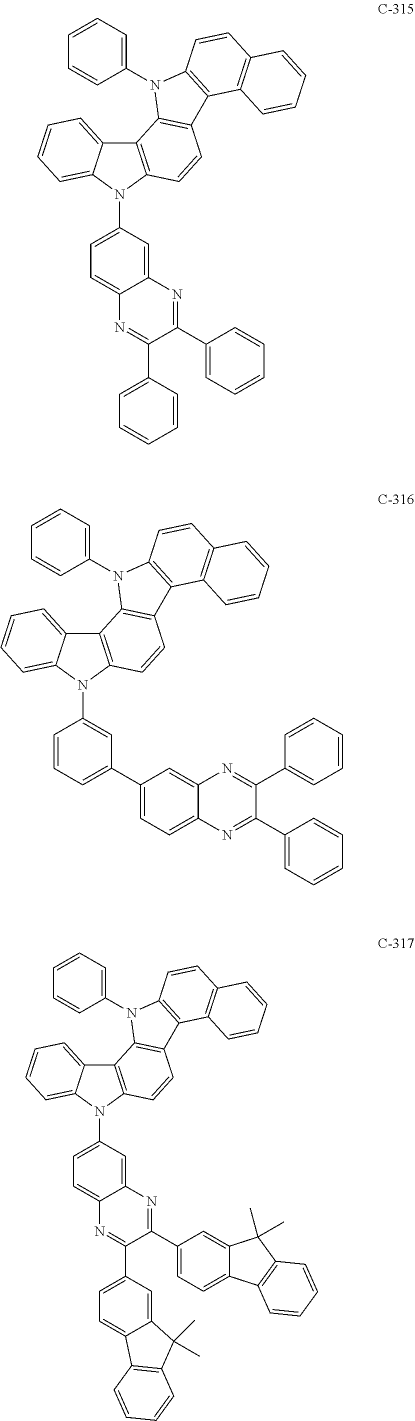 [SMILES: c1ccc(-c2nc3ccc(-n4c5ccccc5c5c4ccc4c6c7ccccc7ccc6n(-c6ccccc6)c45)cc3nc2-c2ccccc2)cc1 ; c1ccc(-c2nc3ccc(-c4cccc(-n5c6ccccc6c6c5ccc5c7c8ccccc8ccc7n(-c7ccccc7)c56)c4)cc3nc2-c2ccccc2)cc1 ; CC1(C)c2ccccc2-c2ccc(-c3nc4ccc(-n5c6ccccc6c6c5ccc5c7c8ccccc8ccc7n(-c7ccccc7)c56)cc4nc3-c3ccc4c(c3)C(C)(C)c3ccccc3-4)cc21]

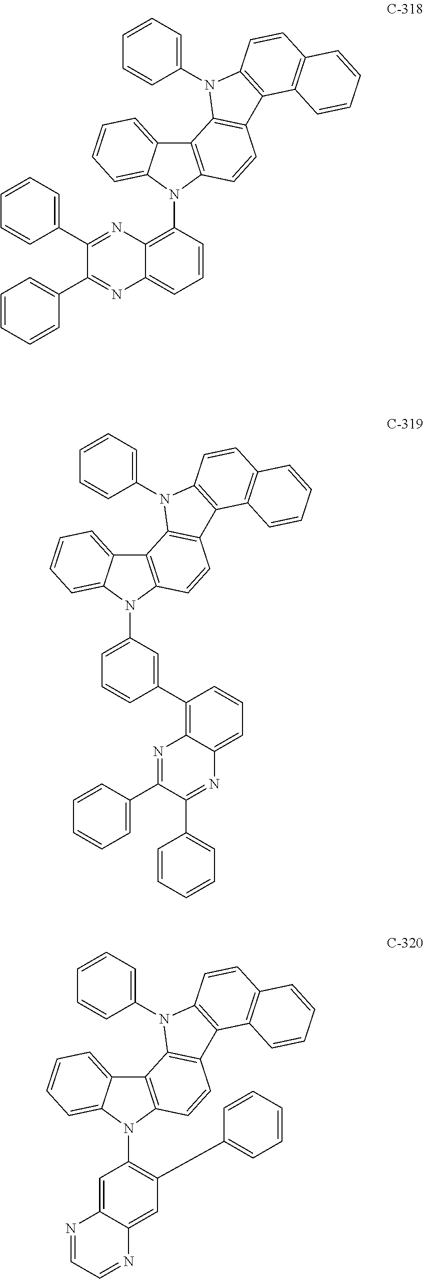 [SMILES: c1ccc(-c2nc3cccc(-n4c5ccccc5c5c4ccc4c6c7ccccc7ccc6n(-c6ccccc6)c45)c3nc2-c2ccccc2)cc1 ; c1ccc(-c2nc3cccc(-c4cccc(-n5c6ccccc6c6c5ccc5c7c8ccccc8ccc7n(-c7ccccc7)c56)c4)c3nc2-c2ccccc2)cc1 ; c1ccc(-c2cc3nccnc3cc2-n2c3ccccc3c3c2ccc2c4c5ccccc5ccc4n(-c4ccccc4)c23)cc1]

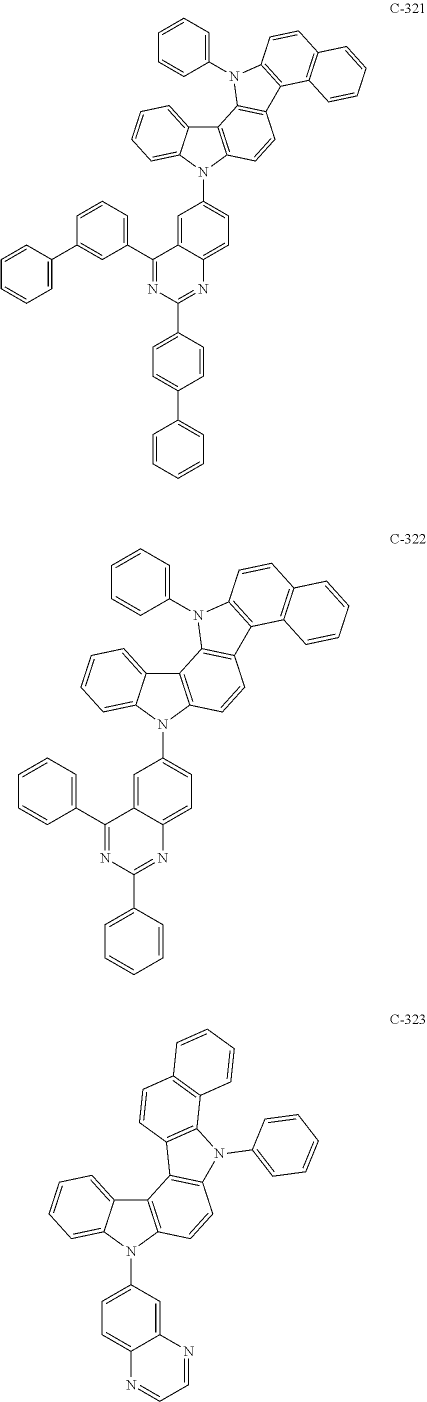 [SMILES: c1ccc(-c2ccc(-c3nc(-c4cccc(-c5ccccc5)c4)c4cc(-n5c6ccccc6c6c5ccc5c7c8ccccc8ccc7n(-c7ccccc7)c56)ccc4n3)cc2)cc1 ; c1ccc(-c2nc(-c3ccccc3)c3cc(-n4c5ccccc5c5c4ccc4c6c7ccccc7ccc6n(-c6ccccc6)c45)ccc3n2)cc1 ; c1ccc(-n2c3ccc4c(c5ccccc5n4-c4ccc5nccnc5c4)c3c3ccc4ccccc4c32)cc1]

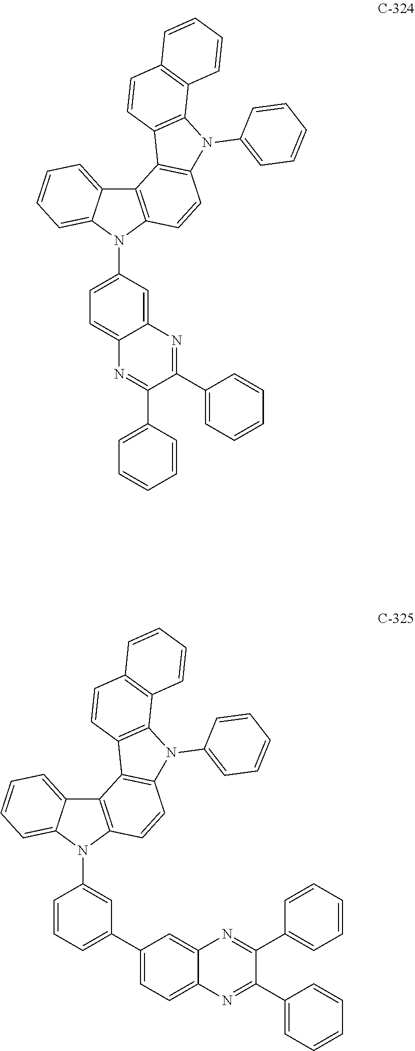 [SMILES: c1ccc(-c2nc3ccc(-n4c5ccccc5c5c6c7ccc8ccccc8c7n(-c7ccccc7)c6ccc54)cc3nc2-c2ccccc2)cc1 ; c1ccc(-c2nc3ccc(-c4cccc(-n5c6ccccc6c6c7c8ccc9ccccc9c8n(-c8ccccc8)c7ccc65)c4)cc3nc2-c2ccccc2)cc1]

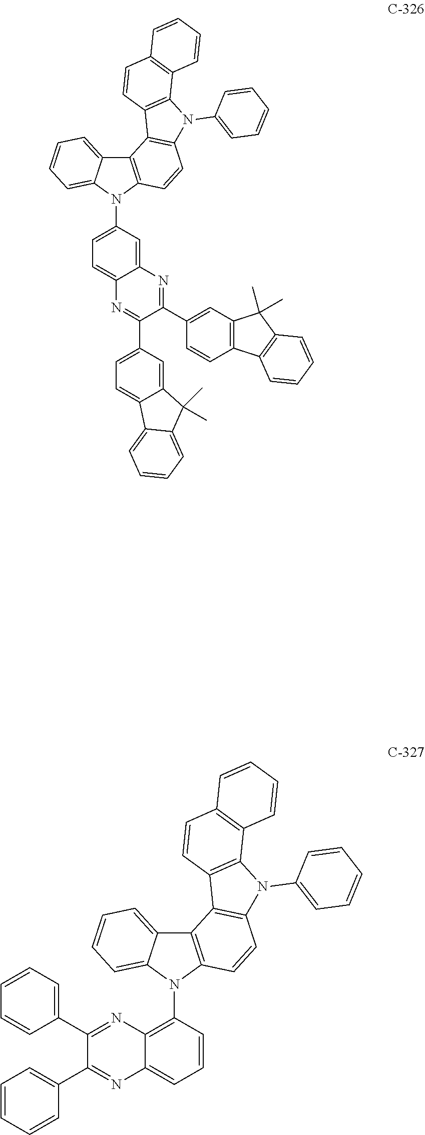 [SMILES: CC1(C)c2ccccc2-c2ccc(-c3nc4ccc(-n5c6ccccc6c6c7c8ccc9ccccc9c8n(-c8ccccc8)c7ccc65)cc4nc3-c3ccc4c(c3)C(C)(C)c3ccccc3-4)cc21 ; c1ccc(-c2nc3cccc(-n4c5ccccc5c5c6c7ccc8ccccc8c7n(-c7ccccc7)c6ccc54)c3nc2-c2ccccc2)cc1]

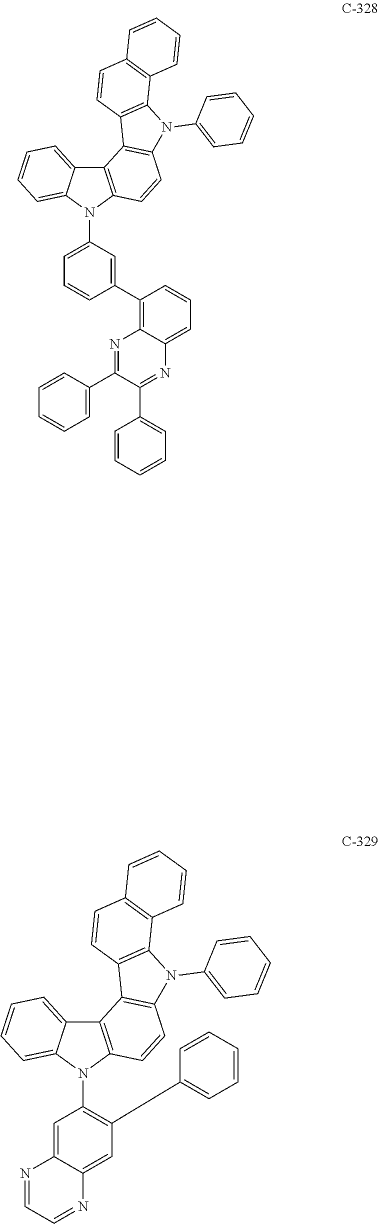 [SMILES: c1ccc(-c2nc3cccc(-c4cccc(-n5c6ccccc6c6c7c8ccc9ccccc9c8n(-c8ccccc8)c7ccc65)c4)c3nc2-c2ccccc2)cc1 ; c1ccc(-c2cc3nccnc3cc2-n2c3ccccc3c3c4c5ccc6ccccc6c5n(-c5ccccc5)c4ccc32)cc1]

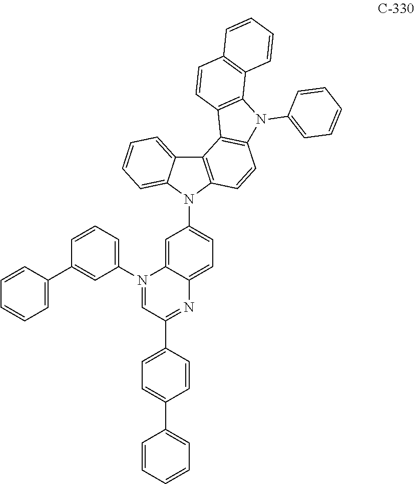

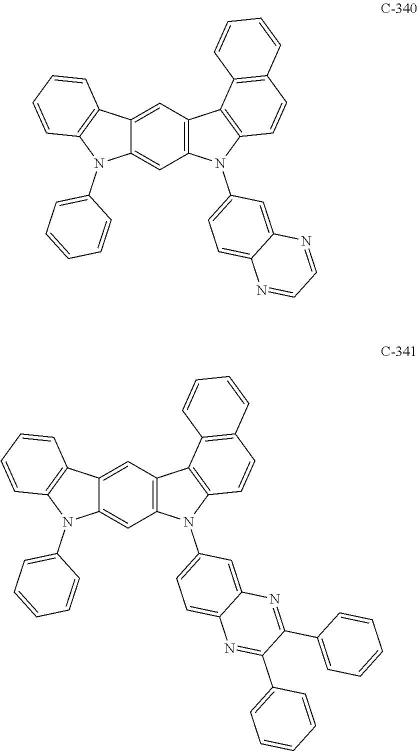 [SMILES: c1ccc(-n2c3ccccc3c3cc4c5c6ccccc6ccc5n(-c5ccc6nccnc6c5)c4cc32)cc1 ; c1ccc(-c2nc3ccc(-n4c5cc6c(cc5c5c7ccccc7ccc54)c4ccccc4n6-c4ccccc4)cc3nc2-c2ccccc2)cc1]

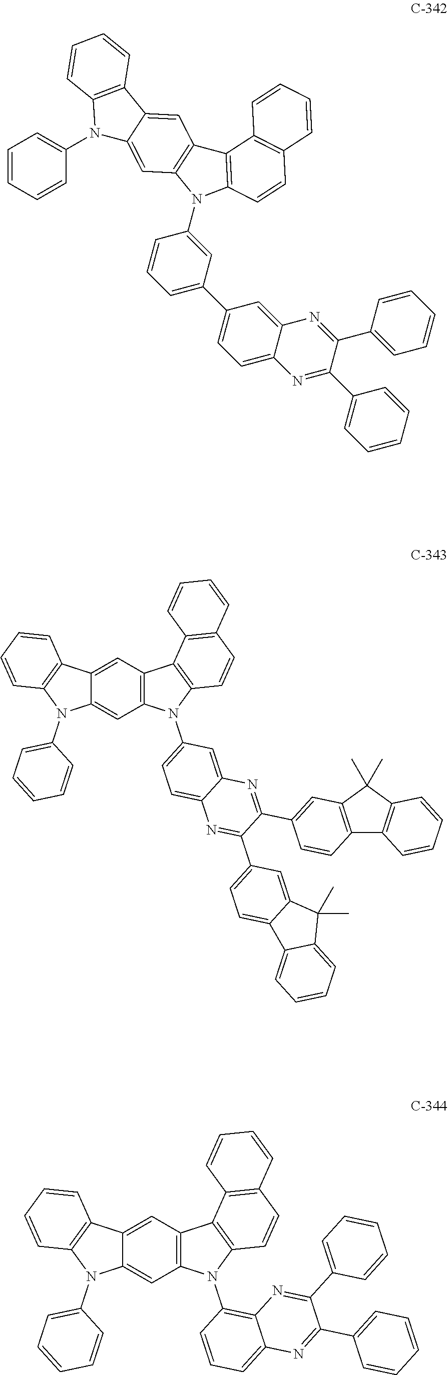 [SMILES: c1ccc(-c2nc3ccc(-c4cccc(-n5c6cc7c(cc6c6c8ccccc8ccc65)c5ccccc5n7-c5ccccc5)c4)cc3nc2-c2ccccc2)cc1 ; CC1(C)c2ccccc2-c2ccc(-c3nc4ccc(-n5c6cc7c(cc6c6c8ccccc8ccc65)c5ccccc5n7-c5ccccc5)cc4nc3-c3ccc4c(c3)C(C)(C)c3ccccc3-4)cc21 ; c1ccc(-c2nc3cccc(-n4c5cc6c(cc5c5c7ccccc7ccc54)c4ccccc4n6-c4ccccc4)c3nc2-c2ccccc2)cc1]

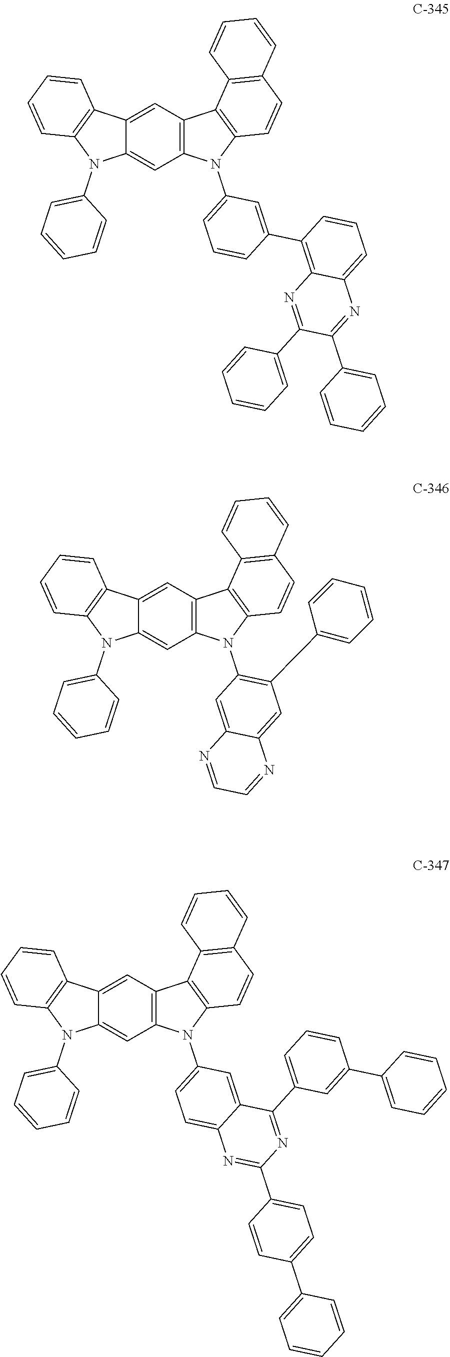 [SMILES: c1ccc(-c2nc3cccc(-c4cccc(-n5c6cc7c(cc6c6c8ccccc8ccc65)c5ccccc5n7-c5ccccc5)c4)c3nc2-c2ccccc2)cc1 ; c1ccc(-c2cc3nccnc3cc2-n2c3cc4c(cc3c3c5ccccc5ccc32)c2ccccc2n4-c2ccccc2)cc1 ; c1ccc(-c2ccc(-c3nc(-c4cccc(-c5ccccc5)c4)c4cc(-n5c6cc7c(cc6c6c8ccccc8ccc65)c5ccccc5n7-c5ccccc5)ccc4n3)cc2)cc1]

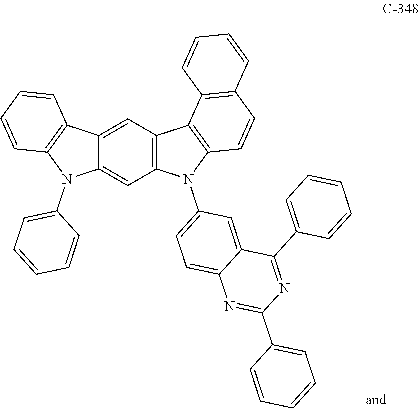 [SMILES: c1ccc(-c2nc(-c3ccccc3)c3cc(-n4c5cc6c(cc5c5c7ccccc7ccc54)c4ccccc4n6-c4ccccc4)ccc3n2)cc1]

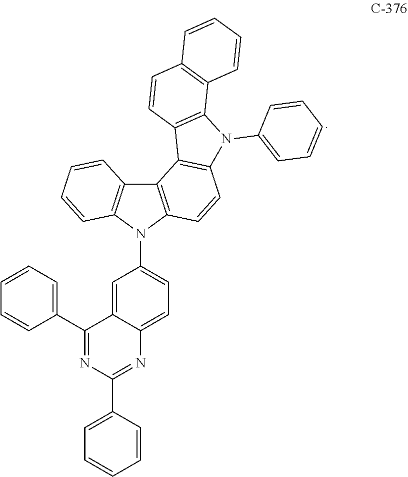 [SMILES: c1ccc(-c2nc(-c3ccccc3)c3cc(-n4c5ccccc5c5c6c7ccc8ccccc8c7n(-c7ccccc7)c6ccc54)ccc3n2)cc1]

7. An organic electroluminescent material comprising the organic electroluminescent compound according to claim 1.

8. An organic electroluminescent device comprising the organic electroluminescent compound according to claim 1.

9. The organic electroluminescent device according to claim 8, wherein the organic electroluminescent compound is comprised in a light-emitting layer.

10. The organic electroluminescent device according to claim 9, wherein the light-emitting layer further comprises an organic electroluminescent compound besides the organic electroluminescent compound, and the further comprised organic electroluminescent compound is represented by the following formula 11: 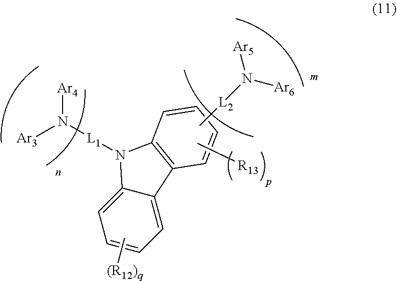
wherein
Ar₃ to Ar₆ each independently represent a substituted or unsubstituted (C6-C30)aryl;
L₁ represents a single bond, or a substituted or unsubstituted (C6-C30)aryl(ene);
L₂ represents a single bond, or a substituted or unsubstituted (C6-C30)arylene;
R₁₂ and R₁₃ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, or a substituted or unsubstituted (C1-C30)alkyl(C6-C30)arylamino; or may be linked to an adjacent substituent to form a ring;
m and n each independently represent an integer of 0 to 2, where at least one of m and n is 1 or more; and
p and q each independently represent an integer of 1 to 4, where p and q are an integer of 2 or more, each R₁ and each R₂ may be the same or different.
